(19) United States
(12) Patent Application Publication
(10) Pub. No.: US 2023/0203485 A1
(43) Pub. Date: Jun. 29, 2023

(54) METHODS FOR MODULATING MHC-I EXPRESSION AND IMMUNOTHERAPY USES THEREOF

(71) Applicants: Patrick C. Lee, Boston, MA (US); Dana-Farber Cancer Institute, Inc., Boston, MA (US)

(72) Inventors: Catherine Wu, Brookline, MA (US); James A. Decaprio, Wellesley, MA (US); Derin B. Keskin, Newton, MA (US); Patrick C. Lee, Boston, MA (US); Sara Buhrlage, Somerville, MA (US)

(21) Appl. No.: 17/928,444

(22) PCT Filed: Jun. 1, 2021
(86) PCT No.: PCT/US21/35205

§ 371 Date: Nov. 29, 2022

Related U.S. Application Data
(60) Provisional application No. 63/039,211, filed on Jun. 15, 2020.
(60) Provisional application No. 63/032,956, filed on Jun. 1, 2020.

(51) Int. Cl. C12N 15/113; A61P 35/00; C07K 16/30

(52) U.S. Cl. CPC C12N 15/113; C12Y 304/19012; A61P 35/00; C07K 16/30; C12N 2310/531; C07K 2317/24; C07K 2317/52

(57) ABSTRACT

The present invention relates, in part, to compositions and methods for modulating major histocompatibility complex (MHC) I expression on cancer cells.

### CROSS-REFERENCE TO RELATED APPLICATIONS

This application claims the benefit of priority to U.S. Provisional Application Ser. No. 63/032,956, filed on 1 Jun. 2020, and U.S. Provisional Application Ser. No. 63/039,211, filed on 15 Jun. 2020; the entire contents of each application are incorporated herein in their entirety by this reference.

### STATEMENT OF RIGHTS

This invention was made with government support under grant numbers R35 CA232128, P01 CA203655, R21 CA216772, NCI-SPORE-2P50CA101942-11A1, R01 CA155010, U24 CA224331, and R01 HL131768 awarded by the National Institutes of Health. The government has certain rights in the invention.

### LARGE FILES

The instant application includes the complete contents of the accompanying 4 lengthy tables, all of which are ASCII text files, as follows: Table 1, submitted herewith as “Table_1_CRISPR_Positive.txt”, created Jun. 15, 2020 and 348,531 bytes in size; Table 2, submitted herewith as “Table_2_CRISPR_Negative.txt”, created Jun. 15, 2020 and 292,318 bytes in size; Table 3, submitted herewith as “Table_3_ORF_Positive.txt”, created Jun. 12, 2020 and 581,299 bytes in size; and Table 4, submitted herewith as “Table_4_ORF_Negative.txt”, created Jun. 12, 2020 and 855,629 bytes in size. All of these 4 tables are hereby incorporated by reference in their entireties.

### BACKGROUND OF THE INVENTION

Viruses employ an array of mechanisms to evade immune system recognition, allowing for undetected infection and replication. A common target for viral immune evasion is the HLA class I (HLA I or MHC I) antigen presentation pathway, which requires the coordinated function of several steps, including peptide processing (PSMB8/LMP2, PSMB9/LMP7), peptide transport from the cytosol to the ER (TAP1, TAP2), and peptide loading to the B2M-HLA I heavy chain (HLA-A, HLA-B, and HLA-C) complex. To perturb this pathway and avoid viral antigen presentation, viruses block HLA I heavy chain insertion into the ER (CMV), resist proteasomal degradation (EBV), interfere with TAP (herpesviruses), or modulate trafficking and turnover of HLA molecules (HIV), among other mechanisms. These strategies by which viruses circumvent immune recognition can shed light on mechanisms of class I presentation and regulation, with relevance to virology and cancer.

For example, Merkel cell carcinoma (MCC), a rare and highly aggressive neuroendocrine skin cancer, poses an intriguing setting to investigate these questions since Merkel cell polyomavirus (MCPyV) is the causative agent of 80% of cases of MCC. MCPyV consists of only two viral antigens: LT, which binds and inactivates RB, and ST, which has a myriad of emerging functions including recruitment of MYCL to chromatin-modifying complexes. Of note, MCC commonly exhibits low HLA I expression, but the mechanism by which this is mediated is unknown. By immunohistochemistry (IHC), 84% of MCC lesions have been reported to exhibit surface HLA I downregulation or loss, and similar findings have been observed in MCC cell lines. However, HLA I surface expression in MCC also appears to be highly plastic, as it can be upregulated in vitro by interferons or histone deacetylase (HDAC) inhibitors. Thus, therapeutic strategies are urgently needed for increasing HLA expression in cancer cells.

### SUMMARY OF THE INVENTION

The present invention is based, at least in part, on the discovery that inhibiting or blocking one or more biomarkers listed in Tables 1-5, such as MYCL or one or more PRC1.1 complex members like PCGF1, BCORL1, and USP7, results in increased expression of MHC class I molecules, such as HLA I molecules, in cancer cells. The present invention involves the modulation (e.g., upregulation or downregulation) of one or more biomarkers listed in Tables 1-5, such as MYCL and/or one or more PRC1.1 complex members (e.g., PCGF1, BCORL1, and USP7) to increase surface expression of MHC class I molecules, such as HLA I molecules, on cancer cells. Using a CRISPR/Cas9-based high throughput screening system and an open reading frame (ORF) screen, the one or more biomarkers listed in Tables 1-5, such as MYCL and/or one or more PRC1.1 complex members (e.g., PCGF1, BCORL1, and USP7) have been identified as targets that, when modulated, sensitize cancers to immunotherapy. For example, in cancers such as Merkel cell cancer, it is demonstrated herein that MHC class I, such as HLA I, surface expression is reduced relative to a control and that upon inhibiting targets like a PRC.1.1 component polypeptide or MYCL, MHC class I, such as HLA I, expression is increased, thereby increasing the susceptibility of these cells to immunotherapies. Functional data validating that one or more biomarkers listed in Tables 1-5, such as MYCL and/or one or more PRC1.1 complex members (e.g., PCGF1, BCORL1, and USP7) inhibition can increase MHC class I, such as HLA I, surface expression is presented herein. Accordingly, modulators of the one or more biomarkers listed in Tables 1-5, such as MYCL and/or one or more PRC1.1 complex members (e.g., PCGF1, BCORL1, and USP7) are useful for modulating MHC class I expression and for modulating immune responses (e.g., increasing or decreasing immune responses), particularly in patients afflicted with cancer, and represents a novel strategy for treating cancer in the setting of concurrent immunotherapy.

One aspect of the invention provides a method of treating a subject afflicted with a cancer comprising administering to the subject a therapeutically effective amount of an agent that modifies the copy number, the expression level, and/or the activity of one or more biomarkers listed in Table 1, 2, 3, 4, or 5 or a fragment thereof, and an immunotherapy.

Numerous embodiments are further provided that can be applied to any aspect of the present invention and/or combined with any other embodiment described herein. For example, in one embodiment, the agent decreases the copy number, the expression level and/or the activity of one or more biomarkers listed in Table 1 or 4 or a fragment thereof. In another embodiment, the agent decreases the copy number, the expression level, and/or the activity of MYCL polypeptide and/or a polycomb repressor complex 1.1 (PRC1.1) polypeptide, or polynucleotide encoding the polypeptide. In still another embodiment, the polycomb repressor complex 1.1 (PRC1.1) polypeptide is USP7, BCORL1, PCGF1, KDM2B, SKP1, RING1A, RING1B, RYBP, YAF2, and/or BCOR. In yet another embodiment, the agent is a small molecule inhibitor, CRISPR single-guide RNA (sgRNA), RNA interfering agent, antisense oligonucleotide, peptide or peptidomimetic inhibitor, aptamer, antibody, or intrabody. In another embodiment, the RNA interfering agent is a small interfering RNA (siRNA), CRISPR RNA (crRNA), a small hairpin RNA (shRNA), a microRNA (miRNA), or a piwi-interacting RNA (piRNA). In still another embodiment, the sgRNA comprises a nucleic acid sequence selected from the group consisting of nucleic acid sequences listed in Tables 1-4. In yet another embodiment, the agent comprises an intrabody, or an antigen binding fragment thereof, that specifically binds to the one or more biomarkers and/or a substrate of the one or more biomarkers listed in Table 1, 2, 3, 4, or 5. In another embodiment, wherein the intrabody, or antigen binding fragment thereof, is a murine, chimeric, humanized, composite, or human intrabody, or antigen binding fragment thereof. In another embodiment, the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabody fragments. In another embodiment, the agent increases the copy number, the expression level and/or the activity of one or more biomarkers listed in Table 2 or 3 or a fragment thereof. In still another embodiment, the agent increases the sensitivity of the cancer cells to an immunotherapy. In yet another embodiment, the immunotherapy is administered before, after, or concurrently with the agent. In still another embodiment, the immunotherapy comprises an anti-cancer vaccine and/or virus. In another embodiment, the immunotherapy is a cell-based immunotherapy, optionally wherein the cell-based immunotherapy is chimeric antigen receptor (CAR-T) therapy. In yet another embodiment, wherein the immunotherapy inhibits an immune checkpoint. In still another embodiment, the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR. In another embodiment, the immune checkpoint is selected from the group consisting of PD-1, PD-L1, and PD-L2, optionally wherein the immune checkpoint is PD-1. In yet another embodiment, the one or more biomarker comprises a nucleic acid sequence having at least 95% identity to a nucleic acid sequence listed in Table 5 and/or encodes an amino acid sequence having at least 95% identity to an amino acid sequence listed in Table 5. In another embodiment, the subject is a mammal. In yet another embodiment, the subject is a human, non-human primate, mouse, rat, or domesticated mammal. In yet another embodiment, the agent increases the sensitivity of the cancer to the immunotherapy, optionally wherein (i) the immunotherapy is T-cell-mediated and/or (ii) the agent increases the amount of CD8+ T cells in a tumor comprising the cancer cells. In another embodiment, the agent increases the level of MHC-I on the surface of the cancer cells. In another embodiment, the method also comprises administering to the subject at least one additional cancer therapy or regimen. In yet another embodiment, the at least one additional cancer therapy or regimen is administered before, after, or concurrently with the agent and/or the immunotherapy. In yet another embodiment, the agent is administered in a pharmaceutically acceptable formulation. In still another embodiment, the cancer is a neuroendocrine cancer. In still another embodiment, the neuroendocrine cancer is a Merkel cell carcinoma, neuroblastoma, small cell lung cancer, or neuroendocrine carcinoma.

Another aspect provides a method of increasing major histocompatibility complex expression in a cancer cell, the method comprising contacting the cancer cell with an agent that modulates the copy number, the expression level, and/or the activity of one or more biomarkers listed in Table 1, 2, 3, 4, or 5 or a fragment thereof, optionally further comprising contacting the cancer cell, or a population of cells comprising the cancer cell and immune cells, with an immunotherapy.

Numerous embodiments are further provided that can be applied to any aspect of the present invention and/or combined with any other embodiment described herein. In one embodiment, the agent that decreases the copy number, the expression level, and/or the activity of one or more biomarkers listed in Table 1 or 4. In another embodiment, the agent decreases the copy number, the expression level, and/or the activity of MYCL polypeptide and/or a polycomb repressor complex 1.1 (PRC1.1) polypeptide, or polynucleotide encoding the polypeptide. In yet another embodiment, the polycomb repressor complex 1.1 (PRC1.1) polypeptide is USP7, BCORL1, PCGF1, KDM2B, SKP1, RING1A, RING1B, RYBP, YAF2, and/or BCOR. In still another embodiment, the agent is a small molecule inhibitor, CRISPR single-guide RNA (sgRNA), RNA interfering agent, antisense oligonucleotide, peptide or peptidomimetic inhibitor, aptamer, antibody, or intrabody. In one embodiment, the RNA interfering agent is a small interfering RNA (siRNA), CRISPR RNA (crRNA), a small hairpin RNA (shRNA), a microRNA (miRNA), or a piwi-interacting RNA (piRNA). In another embodiment, the sgRNA comprises a nucleic acid sequence selected from the group consisting of nucleic acid sequence listed in Tables 1-4. In yet another embodiment, the agent comprises an intrabody, or an antigen binding fragment thereof, that specifically binds to the one or more biomarkers and/or a substrate of the one or more biomarkers listed in Table 1, 2, 3, 4, or 5. In still another embodiment, the intrabody, or antigen binding fragment thereof, is a murine, chimeric, humanized, composite, or human intrabody. In one embodiment, the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabodies fragments. In another embodiment, the agent increases the copy number, the expression level, and/or the activity of one or more biomarkers listed in Table 2 or 3. In yet another embodiment, the agent increases the sensitivity of the cancer cells to the immunotherapy. In yet another embodiment, the cancer cells are contacted with the immunotherapy before, after, or concurrently with the agent. In still another embodiment, the immunotherapy comprises an anti-cancer vaccine and/or virus. In another embodiment, the immunotherapy is a cell-based immunotherapy, optionally wherein the cell-based immunotherapy is chimeric antigen receptor (CAR-T) therapy. In another embodiment, the immunotherapy inhibits an immune checkpoint. In yet another embodiment, the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR. In still another embodiment, the immune checkpoint is selected from the group consisting of PD-1, PD-L1, and PD-L2. In still another embodiment, the immune checkpoint is PD-1. In another embodiment, the biomarker comprises a nucleic acid sequence having at least 95% identity to a nucleic acid sequence listed in Table 5 and/or encodes an amino acid sequence having at least 95% identity to an amino acid sequence listed in Table 5. In yet another embodiment, the one or more biomarker is a human, mouse, chimeric, or a fusion biomarker. In another embodiment, the immunotherapy is (i) T-cell-mediated and/or (ii) the agent increases the amount of CD8+ T cells in a tumor comprising the cancer cells. In yet another embodiment, the agent increases the level of MIC class I surface expression in the cancer cells. In still another embodiment the method further comprises administering to the subject at least one additional cancer therapy or regimen. In another embodiment, the at least one additional cancer therapy or regimen is administered before, after, or concurrently with the agent and/or the immunotherapy. In another embodiment, the agent is administered in a pharmaceutically acceptable formulation. In one embodiment, the cancer cell is a neuroendocrine cancer cell. In another embodiment, the neuroendocrine cancer cell is a Merkel cell carcinoma, neuroblastoma, small cell lung cancer, or neuroendocrine carcinoma cell.

Another aspect of the present invention is a method of identifying a subject afflicted with, or at risk for developing, a cancer that can be treated by modulating the copy number, amount, and/or activity of at least one biomarker listed in Table 1, 2, 3, 4, or 5, the method comprising detecting an increased or decreased level of major histocompatibility complex (MHC) class I expression in a cell from the subject relative to a control, thereby identifying the subject afflicted with, or at risk of developing, a cancer that can be treated by modulating the copy number, amount, and/or activity of at least one biomarker listed in Table 1, 2, 3, 4, or 5, optionally wherein a biological sample comprising the cell from the subject is obtained from the subject.

Numerous embodiments are further provided that can be applied to any aspect of the present invention and/or combined with any other embodiment described herein. In one embodiment, the agent decreases the copy number, amount, and/or activity of at least one biomarker listed in Table 1 or 4. In another embodiment, the method also comprises recommending, prescribing, or administering to the identified subject an agent that inhibits the at least one biomarker listed in Table 1 or 4. In yet another embodiment, the agent increases the copy number, amount, and/or activity of at least one biomarker listed in Table 2 or 3. In another embodiment, the method further comprises recommending, prescribing, or administering to the identified subject an immunotherapy. In one embodiment, the immunotherapy comprises an anti-cancer vaccine, an anti-cancer virus, and/or a checkpoint inhibitor. In another embodiment, the method further comprises recommending, prescribing, or administering to the subject a cancer therapy selected from the group consisting of targeted therapy, chemotherapy, radiation therapy, and/or hormonal therapy. In yet another embodiment, the control comprises a sample derived from a cancerous or non-cancerous sample from either the patient or a member of the same species to which the patient belongs. In still another embodiment, the control is a known reference value. In one embodiment, the cancer is a neuroendocrine cancer. In another embodiment, the neuroendocrine cancer is a Merkel cell carcinoma, neuroblastoma, small cell lung cancer, or neuroendocrine carcinoma.

In another aspect, a method is provided for predicting the clinical outcome of a subject afflicted with a cancer expressing one or more biomarkers listed in Table 1, 2, 3, 4, or 5 or a fragment thereof to treatment with an immunotherapy, the method comprising a) determining the copy number, amount, and/or activity of at least one biomarker listed in Table 1, 2, 3, 4, or 5 in a subject sample; b) determining the copy number, amount, and/or activity of the at least one biomarker in a control having a good clinical outcome; and c) comparing the copy number, amount, and/or activity of the at least one biomarker in the subject sample and in the control; wherein the presence of, or an insignificant change in the copy number, amount, and/or activity of, the at least one biomarker listed in Table 1, 2, 3, 4, or 5 in the subject sample as compared to the copy number, amount and/or activity in the control, is an indication that the subject has a poor clinical outcome.

Another aspect provides a method for monitoring the treatment of a subject having or suspected of having cancer with an agent that decreases the copy number and/or amount and/or inhibits the activity of at least one biomarker listed in Table 1 or 4 and an immunotherapy, the method comprising detecting a change or no change in the level of MHC class I expression in a sample derived from the subject at a first time point and the level of MIC class I expression in a sample derived from the subject at a subsequent time point, thereby monitoring the treatment of the subject.

Yet another aspect provides a method for monitoring the treatment of a subject having or suspected of having cancer with an agent that increases the copy number and/or amount and/or inhibits the activity of at least one biomarker listed in Table 2 or 3 and an immunotherapy, the method comprising detecting a change or no change in the level of MHC class I expression in a sample derived from the subject at a first time point and the level of MIC class I expression in a sample derived from the subject at a subsequent time point, thereby monitoring the treatment of the subject.

In still another aspect, a method is provided for assessing the efficacy of an agent that decreases the copy number, amount, and/or the activity of at least one biomarker listed in Table 1 or 4 in a subject, the method comprising detecting in a subject sample at a first time point a change or no change in the copy number, amount, and/or or activity of at least one biomarker listed in Table 1 or 4 relative to a subsequent time point, wherein a decrease in the copy number, amount, and or activity of at least one biomarker listed in Table 1 or 4 indicates the agent is effective.

Another aspect provides a method of assessing the efficacy of an agent that increases the copy number, amount, and/or the activity of at least one biomarker listed in Table 2 or 3 in a subject, the method comprising detecting in a subject sample at a first time point a change or no change in the copy number, amount, and/or or activity of at least one biomarker listed in Table 2 or 3 relative to a subsequent time point, wherein a decrease in the copy number, amount, and or activity of at least one biomarker listed in Table 2 or 3 indicates the agent is effective.

Numerous embodiments are further provided that can be applied to any aspect of the present invention and/or combined with any other embodiment described herein. In one embodiment, between the first point in time and the subsequent point in time, the subject has undergone treatment, completed treatment, and/or is in remission for the cancer. In another embodiment, treatment comprises administering the agent to the subject. In yet another embodiment, the first and/or the subsequent sample comprises ex vivo or in vivo samples. In still another embodiment, the first and/or at least one subsequent sample is a portion of a single sample or pooled samples obtained from the subject. In another embodiment, the sample comprises cells, serum, peritumoral tissue, and/or intratumoral tissue obtained from the subject. In yet another embodiment, the one or more biomarkers listed in Table 1, 2, 3, 4, or 5. In one embodiment, the cancer or cancer cell is a neuroendocrine cancer. In another embodiment, the neuroendocrine cancer is a Merkel cell carcinoma, neuroblastoma, small cell lung cancer, or neuroendocrine carcinoma. In yet another embodiment, the cancer or cancer cell is in an animal model of the cancer. In still another embodiment, the animal model is a mouse model. In one embodiment, the cancer is in a mammalian subject. In another embodiment, the mammalian subject is a mouse or a human. In yet another embodiment, the mammal is a human.

Although the aspects and embodiments described above provide representative embodiments for biomarkers of the present invention, such as those listed in Tables 1, 4, and 5, for which inhibition in combination with an immunotherapy, results in a synergistic therapeutic benefit for treating cancers that is unexpected given the lack of such benefit observed for the immunotherapy alone, certain biomarkers clearly described herein, especially at Tables 1, 4, and 5, whose promoted expression rather than inhibition in combination with an immunotherapy (e.g., identified as being enriched in the sgRNA screen rather than being depleted), results in a synergistic therapeutic benefit for treating cancers, are readily apparent. Thus, any aspect and embodiment described herein and above can use such biomarkers and their promoted expression in diagnostic, prognostic, therapeutic, etc. applications regarding immunotherapy and cancers. For example, in one aspect, a method of killing cancer cells comprising contacting the cancer cells with an agent that promotes rather than inhibits the copy number, the expression level, and/or the activity of one or more such biomarkers listed in Tables, 1, 4, or 5, or a fragment thereof, in combination with an immunotherapy, is provided. In another representative aspect, a method of determining whether a subject afflicted with a cancer or at risk for developing a cancer would benefit from promoting the copy number, amount, and/or activity of such at least one biomarker listed in Table 1, 4, or 5 is provided, the method comprising a) obtaining a biological sample from the subject; b) determining the copy number, amount, and/or activity of at least one biomarker listed in Table 1; c) determining the copy number, amount, and/or activity of the at least one biomarker in a control; and d) comparing the copy number, amount, and/or activity of the at least one biomarker detected in steps b) and c); wherein the absence of, or a significant decrease in, the copy number, amount, and/or activity of, the at least one biomarker listed in Table 1, 4, or 5 in the subject sample relative to the control copy number, amount, and/or activity of the at least one biomarker indicates that the subject afflicted with the cancer or at risk for developing the cancer would benefit from promoting the copy number, amount, and/or activity of the at least one biomarker listed in Table 1, 4, or 5.

Additionally, although the aspects and embodiments described above provide representative embodiments for biomarkers of the present invention, such as those listed in Tables 2 and 3, for which promotion in combination with an immunotherapy, results in a synergistic therapeutic benefit for treating cancers that is unexpected given the lack of such benefit observed for the immunotherapy alone, certain biomarkers clearly described herein, especially at Tables 2 and 3, whose inhibited expression rather than promotion in combination with an immunotherapy (e.g., identified as being enriched in the sgRNA screen rather than being depleted), results in a synergistic therapeutic benefit for treating cancers, are readily apparent. Thus, any aspect and embodiment described herein and above can use such biomarkers and their promoted expression in diagnostic, prognostic, therapeutic, etc. applications regarding immunotherapy and cancers. For example, in one aspect, a method of killing cancer cells comprising contacting the cancer cells with an agent that promotes rather than inhibits the copy number, the expression level, and/or the activity of one or more such biomarkers listed in Tables, 2 or 3, or a fragment thereof, in combination with an immunotherapy, is provided. In another representative aspect, a method of determining whether a subject afflicted with a cancer or at risk for developing a cancer would benefit from promoting the copy number, amount, and/or activity of such at least one biomarker listed in Table 2 or 3 is provided, the method comprising a) obtaining a biological sample from the subject; b) determining the copy number, amount, and/or activity of at least one biomarker listed in Table 1; c) determining the copy number, amount, and/or activity of the at least one biomarker in a control; and d) comparing the copy number, amount, and/or activity of the at least one biomarker detected in steps b) and c); wherein the absence of, or a significant decrease in, the copy number, amount, and/or activity of, the at least one biomarker listed in Table 2 or 3 in the subject sample relative to the control copy number, amount, and/or activity of the at least one biomarker indicates that the subject afflicted with the cancer or at risk for developing the cancer would benefit from promoting the copy number, amount, and/or activity of the at least one biomarker listed in Table 2 or 3

### BRIEF DESCRIPTION OF THE DRAWINGS

The patent application file contains at least one drawing executed in color. Copies of this patent or patent application publication with color drawing(s) will be provided by the office upon request and payment of the necessary fee.

FIG. 1A-FIG. 1S show the generation of patient-derived MCC lines that exhibit classic features of MCC and recapitulate their corresponding original tumors.

FIG. 1A is a graph showing cell culture media optimization in the MCC-336 cell line. Cells were counted at Day 0, 4, and 7.

FIG. 1B are images showing Immunohistochemistry of MCC cell lines with stains for MCC markers SOX2 and CK20. One representative virus-positive (MCC-277) and virus-negative (MCC-350) line are shown.

FIG. 1C comprises images showing immunohistochemistry for 9 of the newly generated MCC cell lines, with staining for classical MCC markers SOX2 and CK20.

FIG. 1D shows virpanel data.

FIG. 1E is a CoMut plot displaying the top 50 most frequently mutated genes across 7 MCC tumor and cell line pairs.

FIG. 1F shows clustering of MCC tumors and cell lines by mutational profiles. Similarity scores were calculated based on the concordant presence or absence of mutations between tumor and cell line on a 0 to 1 scale, where a score of 1 indicates identical profiles.

FIG. 1G shows unsupervised hierarchical clustering of RNA-seq samples, comprised of 9 MCC patient tumors and corresponding cell lines. Heatmaps were constructed using a distance matrix on variance-stabilizing transformed expression values. Top track indicates quantification of transcript reads mapping to the MCPyV genome.

FIG. 1H is a graph showing pairwise Spearman correlations based on RNA-Seq data for corresponding tumor-cell line pairs, tumor-tumor pairs, cell line-cell line pairs, and all other pairings.

FIG. 1I is a diagram showing translated unannotated ORFs that can be translated by Ribo-seq.

FIG. 1J shows flow cytometry results (left y-axis) for HLA-I surface expression across 11 MCC lines, both at baseline (pink bars) and in response to IFN-γ (red bars), compared to isotype control (white bars). The overlaid black line plot indicates the percentage of tumor cells that stained positive for HLA-I by IHC of the corresponding original tumor (right y-axis).

FIG. 1K shows IHC staining of 4 original MCC tumor biopsies for HLA class I, HLA-DR, CD4, and CD8.

FIG. 1L shows flow cytometry experiments measuring HLA-ABC surface expression (W6/32 antibody) in the MCC-301 line and two established MCPyV+ lines, MKL-1 and WaGa.

FIG. 1M comprises graphs showing the effect of type I and type II interferons on surface MHC I expression in MCC by flow cytometry. 5×105 MCC cells were treated with the indicated doses of IFNα2β, IFNβ, or IFNγ for 24 hours. Representative histogram plots show cells stained with anti-HLA class I or isotype antibodies. The experiment was performed in the MCPyV− line MCC-290 (left) and the MCPyV+ line MCC-301 (right).

FIG. 1N is a graph showing flow cytometry assessment of HLA-DR expression in all 11 MCC lines, both at baseline (light pink) and after IFN-γ treatment (red).

FIG. 1O shows IHC of MCC tumor archival samples. On the left is a summary of the percent of MCC cells that are HLA I-positive within available pre- (n=6) and post-treatment (n=9) tumor samples (see Table 6 for prior treatments). MCC cell lines were derived from post-treatment samples. Representative IHC images of two HLA I-low tumors, MCC-301 and MCC-336 are on the right, stained for HLA class I (brown) with SOX2 co-stain (red) to identify MCC cells.

FIG. 1P shows growth curves of newly generated MCC cell lines. One million cells were seeded in triplicate on Day 0 and counted at Day 2 and Day 4.

FIG. 1Q shows IHC images of parental MCC tumors, stained for HLA class I (brown) with SOX2 co-stain (red) to identify MCC cells.

FIG. 1R shows a summary of the percent of MCC cells that are HLA II-positive within available pre- (n=6) and post-treatment (n=9) tumor samples (see Table 6 for prior treatments). MCC cell lines were derived from post-treatment samples.

FIG. 1S shows representative multiplex immunofluorescence images of MCC FFPE tumor tissue sections. Probes include DAPI nuclear (blue), CD8 (white), FOXP3 (yellow), PD-1 (orange), PD-L1 (green), and SOX2 (magenta).

FIG. 2A-FIG. 2O illustrate that transcriptional suppression of multiple class I pathway genes and NLRC5 alterations underlie the loss of MHC I surface expression in this panel of MCC lines.

FIG. 2A comprises RNA-seq heatmaps of class I antigen presentation gene expression in MCC lines and controls. Counts were normalized by a set of housekeeping genes (Eisenberg and Levanon 2013), using the RUV method (Risso et al. (2014) Nature 32 (9): 896-902. The middle heatmap shows unsupervised clustering by Euclidean distance of the MCC cell line panel, both at baseline and after IFN-γ treatment. The left heatmap is a reference heatmap of previously established MCC lines MKL-1 and WaGa. The right heatmap is a reference heatmap of normal epidermal keratinocytes and dermal fibroblasts.

FIG. 2B is a volcano plot of differentially expressed genes (genes below FDR cutoff 0.01 are shown in yellow) between baseline and IFN-γ-treated MCC cell lines. Differential expression analysis was performed using DESeq2, and negative LFC indicates increased expression in +IFN-γ samples. IFN genes are highlighted in red.

FIG. 2C shows unsupervised clustering of proteomic expression values for class I pathway genes in 4 of the MCC lines, at baseline and after IFN-γ treatment.

FIG. 2D is a proteomics heatmap depicting the relative expression of key IFN-γ pathway components in 4 of the MCC lines, both at baseline and after IFN-γ treatment.

FIG. 2E comprises graphs summarizing a targeted analysis of normalized STAT1 peptide counts (left) and STAT-Y701y phosphosite counts (right) between untreated and IFN-γ-treated cell lines.

FIG. 2F shows scRNA-seq data from MCC-336 (MCPyV⁺) and -350 (MCPyV⁻) fresh tumor samples. UMAP (uniform manifold approximation and projection) visualization of all cells are displayed, colored by cluster (left) and by sample (middle). On the right are expression levels of HLA-A, -B, -C, and B2M across all clusters (clusters 0-5=MCC cells; cluster 6=immune cells).

FIG. 2G comprises charts of scRNA-seq expression of MCC markers SOX2, ATOH1, and synaptophysin (SYP), and immune cell marker PTPRC (CD45) within the MCC-336 and -350 tumor samples.

FIG. 2H comprises graphs showing scRNA-seq expression of additional HLA class I genes across all clusters (clusters 0-5: MCC; cluster 6: immune cells).

FIG. 2I shows NLRC5 copy number loss is common in MCC. Log₂ copy number ratios are displayed for class I antigen presentation genes (left) and for chromosome 16 (right), where NLRC5 is located. Red and blue signify copy number gain and loss, respectively.

FIG. 2J comprises graphs showing Pearson correlation plots between class 1 genes and NLRC5 generated from RNA-seq data from the 11 MCC cell lines. P-values not adjusted for multiple comparisons.

FIG. 2K shows unsupervised clustering of promoter-averaged methylation values of class I pathway genes in 8 of the MCC lines, generated from whole-genome bisulfite sequencing.

FIG. 2L is a graph of ATAC-seq normalized read coverage in 8 of the MCC lines, focusing on the TSS+/−5 kb of class I genes and the housekeeping gene TBP. All datasets including those from GEO and ENCODE were normalized by RPKM (see Methods).

FIG. 2M is a graph comparing the percentage of peaks falling within the union DNase-1 hypersensitivity sites (DHS) between the MCC lines and datasets on Cistrome DB. Comparison to the median level (left) as well as the full distribution (right) are shown.

FIG. 2N is a graph comparing total, 5-fold and 10-fold enriched peak numbers across MCC lines with the median of Cistrome DB datasets. Dashed line represents peak number of 500.

FIG. 2O is graph showing peak conservation across samples.

FIG. 3A-FIG. 3N illustrate that IFNy increases and alters the HLA peptidome.

FIG. 3A comprises graphs showing the frequency of peptides predicted to bind to each HLA allele in tumor and cell line samples for MCC-277, -290, and -301.

FIG. 3B shows the number of detected peptides presented on HLA Class I is low for primary tumor and tumor derived cell lines but increased after IFNγ treatment.

FIG. 3C is heatmap showing the correlation of peptide sequences for tumor, cell line and cell line +IFNγ in motif space.

FIG. 3D comprises pie charts showing the allele distribution of peptides detected in tumor and cell line of MCC 2314.

FIG. 3E comprises graphs showing motif changes for tumor, cell line and cell line +IFNγ samples of MCC290 and 301. This Figure also shows 9mer motif changes between untreated and IFN-γ-treated samples for MCC-290 (MCPyV⁻) and -301 (MCPyV⁺) cell lines.

FIG. 3F comprises graphs showing the allele distribution of peptides detected in cell lines +/−IFNγ. HLA allele distribution of presented peptides detected in cell lines is shown at baseline and after IFN-γ treatment. Each HLA allele is represented by a different color.

FIG. 3G comprises graphs showing the increase of peptide presentation per HLA type upon IFN treatment. The Figure shows a summary of changes in peptides presented per HLA gene upon IFN-γ treatment across all MCC lines analyzed for HLA-A (left), -B (middle), and -C (right).

FIG. 3H comprises graphs showing the allele distribution of peptides detected in cell lines+/−IFNγ.

FIG. 3I comprises graphs showing the increase of peptide presentation per HLA type upon IFN treatment.

FIG. 3J is a readout of the mass spectrum of peptide representing Large T antigen in MCC367.

FIG. 3K shows the number of detected peptides presented on HLA-I in MCC lines at baseline (gray bar) and after IFN-γ treatment (red bar). CL=cell line (left). Correlation heatmap of peptide sequences between MCC lines at baseline and after IFN-γ treatment in motif space (right).

FIG. 3L shows IFN-γ secretion by peripheral blood mononuclear cells (PBMCs) from patient MCC-367 co-cultured in an ELISpot with DMSO, HIV-GAG negative control peptide, autologous MCC-367 tumor cells, or the Large T antigen-derived peptide identified in the MCC-367 HLA peptidome in panel F. Left—ELISpot conditions conducted in triplicate. Right—summary statistics (mean±standard deviation). P values determined by one-way ANOVA followed by post hoc Tukey's multiple comparisons test.

FIG. 3M shows a schematic representation of immunopeptidome workflow. HLA molecules are immunoprecipitated from tumor and cell line material, peptides are eluted from HLA complex and analyzed by LC-MS/MS. After database searching, peptides are assigned to their most likely allele by prediction in HLAthena.

FIG. 3N shows motif changes of 9mers between baseline cell line and IFN-γ-treated cell line samples.

FIG. 4A-FIG. 4Q illustrate paired genome-scale CRISPR and ORF screens to identify known and novel regulators of MHC class I surface expression in MCC.

FIG. 4A shows a genome-scale screening workflow: 150 million MCC-301 cells were transduced with library lentivirus (Brunello CRISPR-KO or human ORFeome v8.14) at low multiplicity of infection, and then selected for 3 days with puromycin. Subsequently, cells were stained with an anti-HLA-ABC antibody (W6/32 clone), and MHC I-high and -low populations (top and bottom 10%) were flow cytometrically sorted. Each screen was repeated in triplicate.

FIG. 4B comprises graphs showing flow cytometric assessment of HLA I surface expression (W6/32 antibody) in MCC-301 cells transduced with the human ORFeome v8.1 library lentivirus, 2 days and 20 days after transduction. Controls include MCC-301 cells transduced with a GFP ORF virus, a no-virus control (media added instead), and untransduced cells.

FIG. 4C is a chart showing the distribution of the log 2-normalized construct scores [log 2 (construct reads/total reads*106+1)] for each sorted population in FIG. 4F.

FIG. 4D shows the results for the gain-of-function ORF screen. Genes were ranked according to their log-fold-change enrichment in MHC I-high versus -low populations. Inset: GSEA analysis displaying select gene sets enriched in the ORF positive hits.

FIG. 4E shows the results for the loss-of-function CRISPR-KO screen. Guide RNA ranks based on log-fold-change enrichment in MHC-I-hi versus low populations were input into the STARS algorithm to generate a gene-level ranking of negative (right) and positive (left) hits. Inset: GSEA analysis displaying select gene sets enriched in CRISPR positive and negative hits. Flow cytometry for surface MHC I in MCC-301 ORF lines.

FIG. 4F shows sorted populations of cells from of the ORF (left) and CRISPR (right) screens.

FIG. 4G is a graph showing average LFC enrichment of the 3 highest-scoring sgRNAs for USP7, BCORL1, and PCGF1, with the distribution of a set of control non-targeting or intergenic sgRNAs shown as a reference.

FIG. 4H shows flow cytometry for surface HLA-I (W6/32 antibody) in MCC-301 (left) and MCC-277 (right) cells transduced with the indicated individual ORFs.

FIG. 4I is a scatterplot of gene-level LFCs (average LFC of all constructs) between two replicates of the ORF screen (top) and CRISPR screen (bottom). Notable screen hits are highlighted in red or blue.

FIG. 4J is a graph summarising flow cytometry results for surface MHC I in MCC-301 PRC1.1 KO lines. MCC-301 cells were transduced with lentivirus containing Cas9 and either control sgRNA or sgRNAs targeting PRC1.1 components BCORL1, PCGF1, or USP7. Cells were selected with puromycin for 3 days, and knockout was confirmed via Sanger sequencing and Western blot or qRT-PCR. Cells were stained with anti-HLA-ABC (W6/32) and analyzed on a BD LSRFortessa. Each condition was repeated in technical triplicate.

FIG. 4K is a schematic of PRC1.1 components and MYCL, with yellow indicating screen hits and green indicating screen hits that have also been reported to interact with MCPyV viral antigens.

FIG. 4L comprises a table and a readout of a TIDE analysis of PRC1.1 KO lines. The table shows the percentage of cells with indels in each knockout line was determined using the TIDE software (Brinkman et al. 2014). The TIDE tracing is an example analysis of the PCGF1-2 KO line in MCC-301.

FIG. 4M shows flow cytometry for surface HLA-I in MKL-1 cells transduced with a dox-inducible control shRNA, MYCL shRNA MYCL, or MYCL shRNA with rescue expression of MYCL. The top panel shows representative flow histograms; the middle panel shows mean MFIs (normalized to corresponding samples not treated with dox) for each condition (n=3); the bottom panel shows western blots for MYCL expression levels in each cell line. P values determined by one-way ANOVA followed by post hoc Tukey's multiple comparisons test.

FIG. 4N shows a RNA-seq volcano plot showing LFC expression in MKL-1 cells expressing a shRNA against MYCL compared to a scrambled control shRNA. Class I APM genes with p_adj<0.05 and log₂-fold change (LFC)>1 are highlighted in red; other notable class I genes are in black.

FIG. 4O shows RNA-seq volcano plot showing LFC expression in WaGa cells expressing an shRNA against both ST and LT antigens, compared to a scrambled control shRNA. Class I APM genes with p_adj<0.05 and LFC>1 are highlighted in red; other notable class I genes in black.

FIG. 4P shows flow cytometry for surface HLA-I in a double guide PCGF1 KO line after IFN-γ treatment.

FIG. 4Q shows western blot quantification of TAP1 and TAP2 in MKL-1 cells in response to varying concentrations of IFN-γ.

FIG. 5A-FIG. 5J illustrate that MYCL suppresses HLA I in MCPyV+ MCC and is relevant in MCPyV−MCC and other cancers.

FIG. 5A is a volcano plot of MYCL shRNA knockdown versus scrambled shRNA control in MCPyV+ MKL-1 cells. Class I genes with p_adj<0.05 and LFC>1 are highlighted in red; other notable class I genes in black.

FIG. 5B shows the enrichment of the GO term GO_ANTIGEN_BINDING in GSEA analysis of gene upregulated in MKL-1 shMYCL cells relative to a scrambled shRNA control (FIG. 3E).

FIG. 5C is a volcano plot of pan-T antigen shRNA knockdown versus scrambled control shRNA in MCPyV+ WaGa line. Class I genes with p_adj<0.05 and LFC>1 are highlighted in red; other notable class I genes in black.

FIG. 5D shows differential expression analysis of MKL-1 cells transduced with one of two shRNAs against EP400 (shEP400-2 or shEP400-3), compared to a scrambled shRNA control. Red indicates HLA-I genes with LFC>1 and p_{adj}<0.01. Triangles indicate genes whose p_{adj} values were reported as zero by DeSeq2, and subsequently plotted at the lowest non-zero p_{adj} value in the dataset.

FIG. 5E shows copy number variations in MYC family genes in 4 of the virus-negative MCC lines for which whole-genome sequencing was performed. CN gains and losses are shown in red and blue, respectively. Gray indicates no CNV data.

FIG. 5F shows unsupervised clustering of RNA-seq expression values of class I pathway genes and MYC family genes across all available cancer cell lines in the Cancer Cell Line Encyclopedia. For each cancer type, the median expression value from all cell lines of that cancer classification was used. Color scale is row-min to row-max.

FIG. 5G comprises heatmaps of an RNA-seq analysis of HLA class I genes and notable screen hits across a cohort of 52 MCC tumors and unsupervised hierarchical clustering heatmap using Pearson correlations. Top track: tumor purity scores for each tumor, generated by ESTIMATE (Yoshihara et al., (2013) Nature Communications 4: 2612). Bottom track: Viral status of tumor (orange=positive; green=negative). Right: Similarity matrices between class I genes and screen hits in VP and VN samples. Blue and red indicate negative and positive Pearson correlation coefficient, respectively, and larger circle size corresponds to smaller p value. P-values not corrected for multiple comparisons.

FIG. 5H shows flow cytometry for surface HLA-I in MCC-301 PRC1.1 KO lines (PCGF1, USP7, and BCORL1). Knockout lines were made using either the highest or second-highest scoring sgRNA for each gene. Western blot for PCGF1 (top) and USP7 (bottom) in WT MCC-301, a control MCC-301 line transduced with a non-targeting sgRNA and Cas9, or the indicated knockout line.

FIG. 5I shows RNA-seq volcano plot showing LFC in gene expression in an MCC-301 PCGF1-KO line compared to MCC-301 transduced with a non-targeting sgRNA and Cas9 control. Inset: GSEA plot demonstrating enrichment of PRC2 targets within genes upregulated in the PCGF1-KO line.

FIG. 5J shows western blot showing TAP1 protein levels in non-targeting control and PCGF1-KO lines at varying IFN-γ concentrations.

FIG. 6A-FIG. 6L illustrate pharmacologic inhibition of PRC1.1 component USP7 upregulates HLA I in MCPyV+ and MCPyV− MCC and mediates MYCL-mediated HLA I suppression.

FIG. 6A is a genome browser view of USP7 and PCGF1 with ChIP-seq tracks for MAX (red), EP-400 (blue), MCPyV ST antigen (pink), and activating histone marks H3K4me3 and H3K27Ac (black).

FIG. 6B is genome browser view of BCOR and BCORL1 with ChIP-seq tracks for MAX (red), EP-400 (blue), MCPyV ST antigen (pink), and activating histone marks H3K4me3 and H3K27Ac (black).

FIG. 6C comprises graphs showing that ChIP-qPCR targets the USP7 and PCGF1 promoters, using MKL-1 chromatin immunoprecipitated with either a MAX (left) or EP400 (right) antibody.

FIG. 6D shows flow cytometry experiments measuring HLA-I surface levels in MCC lines treated with the USP7 inhibitor XL177A or control compound XL177B. Y-axis displays MFI (HLA-ABC) in inhibitor-treated cells, normalized to the mean MFI (HLA-ABC) of DMSO-treated cells. Sample preparation and flow cytometry analysis was performed in technical triplicate for each condition. ** is P<0.01; * is P<0.05; n.s. is P≥0.05.

FIG. 6E comprises a chart and plots showing the CRISPR dependency data from the Cancer Dependency Map (DepMap) (Dempster et al., (2019) bioRxiv, doi.org/10.1101/720243); Meyers et al., (2017) Nature Genetics 49 (12): 1779-84), which was stratified based on TP53 mutation status (TP53-mut (n=532) vs. TP53-wt (n=235)). Left: Pearson correlation coefficients and FDRs of the top genes that are co-dependent with USP7, with Polycomb genes highlighted. Right: Graphical comparison of dependency of USP7 versus Polycomb genes PCGF1 and RING1 in TP53-WT (blue) and TP53-mut cell lines (red).

FIG. 6F is a GSEA analysis of genes based on their degree of co-dependency with USP7 within TP53-mut cancer cell lines, as determined by Pearson correlations (FIG. 6D). Genes exhibiting higher codependency had the highest enrichment for the terms GO_HISTONE_UBIQUITINATION and GO_HISTONE_H2A_UBIQUITINATION

FIG. 6G shows ChIP-qPCR targeting the USP7 and PCGF1 promoters, using MKL-1 chromatin immunoprecipitated with either a MAX (left) or EP400 (right) antibody. Each condition was repeated in triplicate, and p-values were calculated by performing a one-way ANOVA followed by a post hoc Dunnett multiple comparisons test.

FIG. 6H shows HLA I flow cytometry to assess the effect of USP7 inhibitors in MKL-1 p53-WT control lines (left) or p53-KO lines (right). Cells were treated with 100 nM XL177A (red), XL177B (black), or DMSO (light gray).

FIG. 6I shows a heatmap of peptide abundances within the HLA-I-presented peptidomes of MCC-301 cells treated with XL177A (red) or XL177B (black), compared to untreated cells (gray) (n=2 replicates). Only peptides that were significantly differentially expressed between any two treatment groups (determined by two-sample t test) are shown.

FIG. 6J shows that the frequency of peptides presented on each HLA allele in MCC-301 cells treated with XL177A or XL177B, compared to untreated cells.

FIG. 6K shows a western blot for p53 in 3 MKL-1 p53 KO lines compared to control lines (WT, SCR, AAVS1).

FIG. 6L shows distribution of cell cycle phases, determined by flow cytometry, of MKL-1 p53 KO lines treated with XL177A, XL177B, or DMSO.

### DETAILED DESCRIPTION OF THE INVENTION

It has been determined herein that regulators of one or more biomarkers listed in Tables 1-5, such as MYCL or one or more PRC1.1 complex members like PCGF1, BCORL1, and USP7, can be used to modulate surface MIC-I expression on cells, modulate immune responses, and augment tumor immunity and responsiveness to immunotherapies. For example, (a) decreasing the copy number, expression level, and/or activity of one or more biomarkers listed in Table 1 or Table 4 and/or (b) increasing the copy number, expression level, and/or activity of one or more biomarkers listed in Table 2 or Table 3, results in increased MHC-I expression on cells and increased immune responses with increased responsiveness to immunotherapies, which is useful for treating disorders that would benefit from increased immune responses like cancer, infection, and the like. Similarly, (a) increasing the copy number, expression level, and/or activity of one or more biomarkers listed in Table 1 or Table 4 and/or (b) decreasing the copy number, expression level, and/or activity of one or more biomarkers listed in Table 2 or Table 3, results in decreased MIC-I expression on cells and decreased immune responses with decreased responsiveness to immunotherapies, which is useful for treating disorders that would benefit from decreased immune responses like autoimmune disorders.

Thus, in some embodiments, the instant disclosure provides methods of increasing immune responses such as to treat cancers, e.g., those cancer types otherwise not responsive or weakly responsive to immunotherapies, with a combination of a negative regulator of one or more biomarkers listed in Tables 1-5 and an immunotherapy. The present invention provides exemplary RNA interfering agents and small molecules that inhibit such regulators and can be used in the combination therapy and other methods described herein, such as agents that inhibit the function and/or the ability of one or more biomarkers listed in Tables 1-5. Similarly, methods of screening for modulators of such regulators and methods of diagnosing, prognosing, and monitoring cancer involving such inhibitors/immunotherapy combination therapies are provided.

### I. Definitions

The articles “a” and “an” are used herein to refer to one or to more than one (i.e. to at least one) of the grammatical object of the article. By way of example, “an element” means one element or more than one element.

The term “altered amount” or “altered level” refers to increased or decreased copy number (e.g., germline and/or somatic) of a biomarker nucleic acid, e.g., increased or decreased expression level in a cancer sample, as compared to the expression level or copy number of the biomarker nucleic acid in a control sample. The term “altered amount” of a biomarker also includes an increased or decreased protein level of a biomarker protein in a sample, e.g., a cancer sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “altered level of expression” of a biomarker refers to an expression level or copy number of the biomarker in a test sample, e.g., a sample derived from a patient suffering from cancer, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. The altered level of expression is greater or less than the standard error of the assay employed to assess expression or copy number, and is preferably at least 20%, 30%, 40%, 50%, 60%, 70%, 80%, 90%, 100%, 150%, 200%, 300%, 350%, 400%, 500%, 600%, 700%, 800%, 900%, 1000% or more times the expression level or copy number of the biomarker in a control sample (e.g., sample from a healthy subjects not having the associated disease) and preferably, the average expression level or copy number of the biomarker in several control samples. In some embodiments, the level of the biomarker refers to the level of the biomarker itself, the level of a modified biomarker (e.g., phosphorylated biomarker), or to the level of a biomarker relative to another measured variable, such as a control (e.g., phosphorylated biomarker relative to an unphosphorylated biomarker).

The term “altered activity” of a biomarker refers to an activity of the biomarker which is increased or decreased in a disease state, e.g., in a cancer sample, as compared to the activity of the biomarker in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

Unless otherwise specified here within, the terms “antibody” and “antibodies” refers to antigen-binding portions adaptable to be expressed within cells as “intracellular antibodies.” (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J Immunol. Meth. 303:19-39).

Antibodies may be polyclonal or monoclonal; xenogeneic, allogeneic, or syngeneic; or modified forms thereof (e.g. humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the nonhuman antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “assigned score” refers to the numerical value designated for each of the biomarkers after being measured in a patient sample. The assigned score correlates to the absence, presence or inferred amount of the biomarker in the sample. The assigned score can be generated manually (e.g., by visual inspection) or with the aid of instrumentation for image acquisition and analysis. In certain embodiments, the assigned score is determined by a qualitative assessment, for example, detection of a fluorescent readout on a graded scale, or quantitative assessment. In one embodiment, an “aggregate score,” which refers to the combination of assigned scores from a plurality of measured biomarkers, is determined. In one embodiment the aggregate score is a summation of assigned scores. In another embodiment, combination of assigned scores involves performing mathematical operations on the assigned scores before combining them into an aggregate score. In certain, embodiments, the aggregate score is also referred to herein as the “predictive score.”

The term “biomarker” refers to a measurable entity of the present invention that has been determined to be predictive of effects of combinatorial therapies comprising one or more inhibitors of one or more biomarkers listed in Tables 1-5, for example, one or more biomarkers listed in Tables 1-5, such as MYCL and/or one or more PRC1.1 complex members (e.g., PCGF1, BCORL1, and USP7). Biomarkers can include, without limitation, nucleic acids and proteins, including those shown in the Tables, the Examples, the Figures, and otherwise described herein. As described herein, any relevant characteristic of a biomarker can be used, such as the copy number, amount, activity, location, modification (e.g., phosphorylation), and the like.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. Unless otherwise stated, the terms include metaplasias. In some embodiments, such cells exhibit such characteristics in part or in full due to the expression and activity of signaling pathways regulated by one or more biomarkers listed in Tables 1-5. In some embodiments, the cancer cells described herein are not sensitive to at least one of immunotherapies. In some embodiments, the cancer cells are treatable with an agent capable of antagonizing regulators of the biomarkers described herein, such as inhibiting expression and/or function, as described herein.

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., Merkel cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

As used herein, a “neuroendocrine cancer” or “neuroendocrine tumor” is either one which arises from the neuroendocrine system or from non-endocrine cells that acquire properties of neuroendocrine cells through an oncogenic process. Most adult neuroendocrine tumors arise from a known primary site, including the carcinoid, pheochromocytoma, and Merkel's cell tumors. Carcinoid tumors can be benign or malignant. Carcinoid cancers include stomach, pancreas, colon, liver, lung (e.g., small cell carcinoma), ovarian, breast, testicular, and cervical cancer. Small cell carcinoma originates in large central with a propensity to metastasize early and often. Pheochromocytoma is a cancer of the adrenal medulla, which causes overproduction of catecholamine by the adrenal gland. Merkel cell carcinoma, a neuroendocrine cancer of the skin, is a cancer that forms on or beneath the skin. Merkle cell cancers may arise from soft tissues underlying the skin and are fast-growing and often spread to other parts of the body.

In certain embodiments, the cancer encompasses Merkle cell carcinoma. MCC was first described in 1972 by Toker as a trabecular carcinoma of the skin with carcinoid features (Toker (1972) Arch. Dermatol. 105:107-110). Toker later reported the presence of neurosecretory granules, membrane bound granules containing dense cores, within the tumor cells. This feature is indistinguishable from tumor cells of neural crest origin and is also present in normal Merkel cells (Tang et al. (1978) Cancer 42:2311-2321). The tumor name was changed to Merkel cell carcinoma to reflect the similarity in appearance of tumor cells to Merkel cells (Toker (1982) Dermatopathol. 4:497-497-500; Rywlin (1982) Am. J. Dermatolpathol. 4:513-515).

MCC is an aggressive neuroendocrine carcinoma of the skin that frequently metastasizes to draining lymph nodes and distant organs including liver, bone, pancreas, lung, and brain (Lewis et al. (2020) Cancer Med. 9:1374-1382). MCC typically presents as a rapidly growing, erythematous lesion, in the dermal layer of the skin. The most common presentation of MCC is in older, fair skin, adults with a lifelong history of intense UV exposure from the sun. MCC occurs less frequently in non-sun-exposed skin as well as in children, young adults, and dark skin persons. Latitude closer to the equator is associated with increased incidence of MCC in North American men, but not women, possibly due to occupational sunlight exposure patterns (Stang et al. (2018) Eur. J. Cancer 94:47-60). Risk for developing MCC is also increased in patients with severely immunocompromising conditions including HIV/AIDS or from medical treatment of auto-immune diseases, solid organ transplantation, and other types of cancers (Becker et al. (2017) Nat. Rev. Dis. Primers 3:17077). The AEIOU mnemonic accounts for 90% of all MCC presentation: Asymptomatic/lack of tenderness, Expanding rapidly, Immune suppression, Older than 50 years, and Ultraviolet-exposed/fair skin (Heath et al. (2008) J. Am. Acad. Dermatol. 58:375-381).

The most recent MCC staging system from the American Joint Committee on Cancer (AJCC), 8th edition, estimates a 5-year overall survival of 51% for local disease, 35% for nodal involvement, and 14% for metastatic disease (Harms et al. (2016) Ann. Surg. Oncol. 23:3564-3571; Trinidad et al. (2019) J. Clin. Pathol. 72:337-340). Surgery and radiation therapy can be curative for local and nodal MCC but systemic therapy is usually required for extensive, metastatic, and recurrent disease. Cytotoxic chemotherapy, based on cisplatin and etoposide regimens, has a high response rate but is limited by a short duration with a mean progression free survival of just 94 days (Iyer et al. (2016) Cancer Med. 5:2294-2301). A revolution in MCC care began when it was determined that checkpoint blockade therapy with antibodies to PD-1 or PD-L1 could induce frequent and durable responses (Nghiem et al. (2016) N. Engl. J. Med. 374:2542-2552; Kaufman et al. (2016) Lancet Oncol. 17:1374-1385; D'Angelo et al. (2018) JAMA Oncol. 4:e180077; Nghiem et al. (2019) J. Clin. Oncol. 37:693-702). Predictions for overall survival may improve as experience with checkpoint blockade therapy increases.

MCC can vary from a pure neuroendocrine histology to a variant form with mixed histologic features. High-grade neuroendocrine MCC cells have a high nuclear to cytoplasmic ratio with scant cytoplasm, giving it the appearance of a small blue cell tumor when stained by hematoxylin and eosin. The tumor nuclei have an open, pepper and salt-appearing chromatin pattern with frequent mitotic figures indicative of a high proliferative rate). Immunohistochemistry (IHC) staining of MCC for neuroendocrine markers are typically positive for chromogranin, synaptophysin, CD56, and neurofilament. MCC also stain specifically for CK20 that typically shows a paranuclear dot-like pattern. In contrast, CK20 staining in normal Merkel cells is more uniformly distributed throughout the cytoplasm. CK20 staining can distinguish MCC from other more common neuroendocrine tumors such as small cell lung carcinoma (SCLC) (Leech et al. (2001) J. Clin. Pathol. 54:727-729). SCLC stains positive for TTF-1 (thyroid-specific transcription factor 1, encoded by the NKX2-1 gene), while MCC is negative for this stain. INSM1 is a useful IHC marker for MCC and Merkel cells, as well as for other neuroendocrine carcinomas (Lilo et al. (2018) Am. J Surg. Pathol. 42:1541-1548).

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In one embodiment, the control may comprise normal or noncancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the present invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a biomarker nucleic acid or “normal” level of expression of a biomarker nucleic acid or protein is the activity/level of expression or copy number in a biological sample, e.g., a sample containing tissue, whole blood, serum, plasma, buccal scrape, saliva, cerebrospinal fluid, urine, stool, and bone marrow, from a subject, e.g., a human, not afflicted with cancer, or from a corresponding non-cancerous tissue in the same subject who has cancer. As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal can result in cytokine secretion, e.g., in a T cell that has received a T cell-receptor-mediated signal. Immune cells that have received a cell-receptor mediated signal, e.g., via an activating receptor are referred to herein as “activated immune cells.”

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “diagnosing cancer” includes the use of the methods, systems, and code of the present invention to determine the presence or absence of a cancer or subtype thereof in an individual. The term also includes methods, systems, and code for assessing the level of disease activity in an individual.

A molecule is “fixed” or “affixed” to a substrate if it is covalently or non-covalently associated with the substrate such that the substrate can be rinsed with a fluid (e.g. standard saline citrate, pH 7.4) without a substantial fraction of the molecule dissociating from the substrate.

The term “expression signature” or “signature” refers to a group of one or more coordinately expressed biomarkers related to a measured phenotype. For example, the genes, proteins, metabolites, and the like making up this signature may be expressed in a specific cell lineage, stage of differentiation, or during a particular biological response. The biomarkers can reflect biological aspects of the tumors in which they are expressed, such as the cell of origin of the cancer, the nature of the non-malignant cells in the biopsy, and the oncogenic mechanisms responsible for the cancer. Expression data and gene expression levels can be stored on computer readable media, e.g., the computer readable medium used in conjunction with a microarray or chip reading device. Such expression data can be manipulated to generate expression signatures.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

The term “immunotherapy” or “immunotherapies” refer to any treatment that uses certain parts of a subject's immune system to fight diseases such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

The term “immunogenic chemotherapy” refers to any chemotherapy that has been demonstrated to induce immunogenic cell death, a state that is detectable by the release of one or more damage-associated molecular pattern (DAMP) molecules, including, but not limited to, calreticulin, ATP and HMGB1 (Kroemer et al. (2013), Annu. Rev. Immunol., 31:51-72). Specific representative examples of consensus immunogenic chemotherapies include 5′-fluorouracil, anthracyclines, such as doxorubicin, and the platinum drug, oxaliplatin, among others.

In some embodiments, immunotherapy comprises inhibitors of one or more immune checkpoints. The term “immune checkpoint” refers to a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein. In one embodiment, the immune checkpoint is PD-1.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

The nucleic acid and amino acid sequences of a representative human PD-1 biomarker is available to the public at the GenBank database under NM_005018.2 and NP_005009.2 and is shown in Table 1 (see also Ishida et al. (1992) 20 EMBO J 11:3887; Shinohara et al. (1994) Genomics 23:704; U.S. Pat. No. 5,698,520). PD-1 has an extracellular region containing immunoglobulin superfamily domain, a transmembrane domain, and an intracellular region including an immunoreceptor tyrosine-based inhibitory motif (ITIM) (Ishida et al. (1992) EMBO J. 11:3887; Shinohara et al. (1994) Genomics 23:704; and U.S. Pat. No. 5,698,520) and an immunoreceptor tyrosine-based switch motif (ITSM). These features also define a larger family of polypeptides, called the immunoinhibitory receptors, which also includes gp49B, PIR-B, and the killer inhibitory receptors (KIRs) (Vivier and Daeron (1997) Immunol. Today 18:286). It is often assumed that the tyrosyl phosphorylated ITIM and ITSM motif of these receptors interacts with SH2-domain containing phosphatases, which leads to inhibitory signals. A subset of these immunoinhibitory receptors bind to MHC polypeptides, for example the KIRs, and CTLA4 binds to B7-1 and B7-2. It has been proposed that there is a phylogenetic relationship between the MHC and B7 genes (Henry et al. (1999) Immunol. Today 20(6):285-8). Nucleic acid and polypeptide sequences of PD-1 orthologs in organisms other than humans are well-known and include, for example, mouse PD-1 (NM_008798.2 and NP_032824.1), rat PD-1 (NM_001106927.1 and NP_001100397.1), dog PD-1 (XM_543338.3 and XP_543338.3), cow PD-1 (NM_001083506.1 and NP_001076975.1), and chicken PD-1 (XM_422723.3 and XP_422723.2).

PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells.

The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell costimulatory or inhibitory signals, and the ability to modulate the immune response.

The term “PD-1 ligand” refers to binding partners of the PD-1 receptor and includes both PD-L1 (Freeman et al. (2000) J. Exp. Med. 192:1027-1034) and PD-L2 (Latchman et al. (2001) Nat. Immunol. 2:261). At least two types of human PD-1 ligand polypeptides exist. PD-1 ligand proteins comprise a signal sequence, and an IgV domain, an IgC domain, a transmembrane domain, and a short cytoplasmic tail. Both PD-L1 (See Freeman et al. (2000) for sequence data) and PD-L2 (See Latchman et al. (2001) Nat. Immunol. 2:261 for sequence data) are members of the B7 family of polypeptides. Both PD-L1 and PD-L2 are expressed in placenta, spleen, lymph nodes, thymus, and heart. Only PD-L2 is expressed in pancreas, lung and liver, while only PD-L1 is expressed in fetal liver. Both PD-1 ligands are upregulated on activated monocytes and dendritic cells, although PD-L1 expression is broader. For example, PD-L1 is known to be constitutively expressed and upregulated to higher levels on murine hematopoietic cells (e.g., T cells, B cells, macrophages, dendritic cells (DCs), and bone marrow-derived mast cells) and non-hematopoietic cells (e.g., endothelial, epithelial, and muscle cells), whereas PD-L2 is inducibly expressed on DCs, macrophages, and bone marrow-derived mast cells (see Butte et al. (2007) Immunity 27:111).

PD-1 ligands comprise a family of polypeptides having certain conserved structural and functional features. The term “family” when used to refer to proteins or nucleic acid molecules, is intended to mean two or more proteins or nucleic acid molecules having a common structural domain or motif and having sufficient amino acid or nucleotide sequence homology, as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics. PD-1 ligands are members of the B7 family of polypeptides. The term “B7 family” or “B7 polypeptides” as used herein includes costimulatory polypeptides that share sequence homology with B7 polypeptides, e.g., with B7-1, B7-2, B7h (Swallow et al. (1999) Immunity 11:423), and/or PD-1 ligands (e.g., PD-L1 or PD-L2). For example, human B7-1 and B7-2 share approximately 26% amino acid sequence identity when compared using the BLAST program at NCBI with the default parameters (Blosum62 matrix with gap penalties set at existence 11 and extension 1 (See the NCBI website). The term B7 family also includes variants of these polypeptides which are capable of modulating immune cell function. The B7 family of molecules share a number of conserved regions, including signal domains, IgV domains and the IgC domains. IgV domains and the IgC domains are art-recognized Ig superfamily member domains. These domains correspond to structural units that have distinct folding patterns called Ig folds. Ig folds are comprised of a sandwich of two 13 sheets, each consisting of anti-parallel 13 strands of 5-10 amino acids with a conserved disulfide bond between the two sheets in most, but not all, IgC domains of Ig, TCR, and MHC molecules share the same types of sequence patterns and are called the C1-set within the Ig superfamily. Other IgC domains fall within other sets. IgV domains also share sequence patterns and are called V set domains. IgV domains are longer than IgC domains and contain an additional pair of β strands.

Preferred B7 polypeptides are capable of providing costimulatory or inhibitory signals to immune cells to thereby promote or inhibit immune cell responses. For example, B7 family members that bind to costimulatory receptors increase T cell activation and proliferation, while B7 family members that bind to inhibitory receptors reduce costimulation. Moreover, the same B7 family member may increase or decrease T cell costimulation. For example, when bound to a costimulatory receptor, PD-1 ligand can induce costimulation of immune cells or can inhibit immune cell costimulation, e.g., when present in soluble form. When bound to an inhibitory receptor, PD-1 ligand polypeptides can transmit an inhibitory signal to an immune cell.

Preferred B7 family members include B7-1, B7-2, B7h, PD-L1 or PD-L2 and soluble fragments or derivatives thereof. In one embodiment, B7 family members bind to one or more receptors on an immune cell, e.g., CTLA4, CD28, ICOS, PD-1 and/or other receptors, and, depending on the receptor, have the ability to transmit an inhibitory signal or a costimulatory signal to an immune cell, preferably a T cell.

Modulation of a costimulatory signal results in modulation of effector function of an immune cell. Thus, the term “PD-1 ligand activity” includes the ability of a PD-1 ligand polypeptide to bind its natural receptor(s) (e.g. PD-1 or B7-1), the ability to modulate immune cell costimulatory or inhibitory signals, and the ability to modulate the immune response.

The term “PD-L1” refers to a specific PD-1 ligand. Two forms of human PD-L1 molecules have been identified. One form is a naturally occurring PD-L1 soluble polypeptide, i.e., having a short hydrophilic domain and no transmembrane domain, and is referred to herein as PD-L1S. The second form is a cell-associated polypeptide, i.e., having a transmembrane and cytoplasmic domain, referred to herein as PD-L1M. The nucleic acid and amino acid sequences of representative human PD-L1 biomarkers regarding PD-L1M are also available to the public at the GenBank database under NM_014143.3 and NP_054862.1. PD-L1 proteins comprise a signal sequence, and an IgV domain and an IgC domain. The signal sequence of PD-L1S is shown from about amino acid 1 to about amino acid 18. The signal sequence of PD-L1M is shown from about amino acid 1 to about amino acid 18. The IgV domain of PD-L1S is shown from about amino acid 19 to about amino acid 134 and the IgV domain of PD-L1M is shown from about amino acid 19 to about amino acid 134. The IgC domain of PD-L1S is shown from about amino acid 135 to about amino acid 227 and the IgC domain of PD-L1M is shown from about amino acid 135 to about amino acid 227. The hydrophilic tail of the PD-L1 exemplified in PD-L1S comprises a hydrophilic tail shown from about amino acid 228 to about amino acid 245. The PD-L1 polypeptide exemplified in PD-L1M comprises a transmembrane domain shown from about amino acids 239 to about amino acid 259 and a cytoplasmic domain shown from about 30 amino acid 260 to about amino acid 290. In addition, nucleic acid and polypeptide sequences of PD-L1 orthologs in organisms other than humans are well-known and include, for example, mouse PD-L1 (NM_021893.3 and NP_068693.1), rat PD-L1 (NM 001191954.1 and NP_001178883.1), dog PD-L1 (XM_541302.3 and XP_541302.3), cow PD-L1 (NM_001163412.1 and NP_001156884.1), and chicken PD-L1 (XM_424811.3 and XP_424811.3).

The term “PD-L2” refers to another specific PD-1 ligand. PD-L2 is a B7 family member expressed on various APCs, including dendritic cells, macrophages and bone-marrow derived mast cells (Zhong et al. (2007) Eur. J. Immunol. 37:2405). APC-expressed PD-L2 is able to both inhibit T cell activation through ligation of PD-1 and costimulate T cell activation, through a PD-1 independent mechanism (Shin et al. (2005) J. Exp. Med. 201:1531). In addition, ligation of dendritic cell-expressed PD-L2 results in enhanced dendritic cell cytokine expression and survival (Radhakrishnan et al. (2003) J. Immunol. 37:1827; Nguyen et al. (2002) J. Exp. Med. 196:1393). The nucleic acid and amino acid sequences of representative human PD-L2 biomarkers are well-known in the art and are also available to the public at the GenBank database under NM_025239.3 and NP_079515.2. PD-L2 proteins are characterized by common structural elements. In some embodiments, PD-L2 proteins include at least one or more of the following domains: a signal peptide domain, a transmembrane domain, an IgV domain, an IgC domain, an extracellular domain, a transmembrane domain, and a cytoplasmic domain. For example, amino acids 1-19 of PD-L2 comprises a signal sequence. As used herein, a “signal sequence” or “signal peptide” serves to direct a polypeptide containing such a sequence to a lipid bilayer, and is cleaved in secreted and membrane bound polypeptides and includes a peptide containing about 15 or more amino acids which occurs at the N-terminus of secretory and membrane bound polypeptides and which contains a large number of hydrophobic amino acid residues. For example, a signal sequence contains at least about 10-30 amino acid residues, preferably about 15-25 amino acid residues, more preferably about 18-20 amino acid residues, and even more preferably about 19 amino acid residues, and has at least about 35-65%, preferably about 38-50%, and more preferably about 40-45% hydrophobic amino acid residues (e.g., valine, leucine, isoleucine or phenylalanine). In another embodiment, amino acid residues 220-243 of the native human PD-L2 polypeptide and amino acid residues 201-243 of the mature polypeptide comprise a transmembrane domain. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta, W. N. et al. (1996) Annu. Rev. Neurosci. 19: 235-263. In still another embodiment, amino acid residues 20-120 of the native human PD-L2 polypeptide and amino acid residues 1-101 of the mature polypeptide comprise an IgV domain. Amino acid residues 121-219 of the native human PD-L2 polypeptide and amino acid residues 102-200 of the mature polypeptide comprise an IgC domain. As used herein, IgV and IgC domains are recognized in the art as Ig superfamily member domains. These domains correspond to structural units that have distinct folding patterns called Ig folds. Ig folds are comprised of a sandwich of two 8 sheets, each consisting of antiparallel (3 strands of 5-10 amino acids with a conserved disulfide bond between the two sheets in most, but not all, domains. IgC domains of Ig, TCR, and MHC molecules share the same types of sequence patterns and are called the Cl set within the Ig superfamily. Other IgC domains fall within other sets. IgV domains also share sequence patterns and are called V set domains. IgV domains are longer than C-domains and form an additional pair of strands. In yet another embodiment, amino acid residues 1-219 of the native human PD-L2 polypeptide and amino acid residues 1-200 of the mature polypeptide comprise an extracellular domain. As used herein, the term “extracellular domain” represents the N-terminal amino acids which extend as a tail from the surface of a cell. An extracellular domain of the present invention includes an IgV domain and an IgC domain, and may include a signal peptide domain. In still another embodiment, amino acid residues 244-273 of the native human PD-L2 polypeptide and amino acid residues 225-273 of the mature polypeptide comprise a cytoplasmic domain. As used herein, the term “cytoplasmic domain” represents the C-terminal amino acids which extend as a tail into the cytoplasm of a cell. In addition, nucleic acid and polypeptide sequences of PD-L2 orthologs in organisms other than humans are well-known and include, for example, mouse PD-L2 (NM_021396.2 and NP_067371.1), rat PD-L2 (NM_001107582.2 and NP_001101052.2), dog PD-L2 (XM_847012.2 and XP_852105.2), cow PD-L2 (XM_586846.5 and XP_586846.3), and chimpanzee PD-L2 (XM_001140776.2 and XP_001140776.1).

The term “PD-L2 activity,” “biological activity of PD-L2,” or “functional activity of PD-L2,” refers to an activity exerted by a PD-L2 protein, polypeptide or nucleic acid molecule on a PD-L2-responsive cell or tissue, or on a PD-L2 polypeptide binding partner, as determined in vivo, or in vitro, according to standard techniques. In one embodiment, a PD-L2 activity is a direct activity, such as an association with a PD-L2 binding partner. As used herein, a “target molecule” or “binding partner” is a molecule with which a PD-L2 polypeptide binds or interacts in nature, such that PD-L2-mediated function is achieved. In an exemplary embodiment, a PD-L2 target molecule is the receptor RGMb. Alternatively, a PD-L2 activity is an indirect activity, such as a cellular signaling activity mediated by interaction of the PD-L2 polypeptide with its natural binding partner (i.e., physiologically relevant interacting macromolecule involved in an immune function or other biologically relevant function), e.g., RGMb. The biological activities of PD-L2 are described herein. For example, the PD-L2 polypeptides of the present invention can have one or more of the following activities: 1) bind to and/or modulate the activity of the receptor RGMb, PD-1, or other PD-L2 natural binding partners, 2) modulate intra- or intercellular signaling, 3) modulate activation of immune cells, e.g., T lymphocytes, and 4) modulate the immune response of an organism, e.g., a mouse or human organism.

“Anti-immune checkpoint therapy” refers to the use of agents that inhibit immune checkpoint nucleic acids and/or proteins. Inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can bind to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy).

The term “USP7,” also known as “Ubiquitin Specific Peptidase 7,” refers to a member of the C19 peptidase family that includes ubiquitinyl hydrolases. USP7 deubiquitinates target proteins (e.g., FOXO4, p53/TP53, MDM2, ERCC6, DNMT1, UHRF1, PTEN, KMT2E/MLL5 and DAXX), which prevents degradation of the deubiquitinated target protein. Thus, USP7 counteracts the activity of ubiquitin ligases.

The nucleic acid and amino acid sequences of a representative human USP7 is available to the public at the GenBank database (Gene ID 7874) and is shown in Table 1. Multiple transcript variants encoding several different isoforms have been found for USP7. Human USP7 variants include the transcript variant 1 encoding isoform 1 (NM_003470.3 and NP_003461.2), the transcript variant 2 encoding isoform 2 (NM_001286457.2 and NP_001273386.2), the transcript variant 3 encoding isoform 3 (NM_001286458.2 and NP_001273387.1), and the transcript variant 4 encoding isoform 4 (NM_001321858.1 and NP_001308787.1).

Nucleic acid and polypeptide sequences of USP7 orthologs in organisms other than humans are well known and include, for example, chimpanzee (XM_024349753.1 and XP_024205521.1; XM_016929384.2 and XP_016784873.1; XM_016929385.2 and XP_016784874.1; and XM_016929388.2 and XP_016784877.1), macaque (XM_015125591.2 and XP_014981077.1; XM_015125592.2 and XP_014981078.1; XM_002802389.3 and XP_002802435.1; and XM_002802388.3 and XP_002802434.1), wolf (XM_005621558.3 and XP_005621615.1; and XM_005621559.3 and XP_005621616.1), cow (XM_024985414.1 and XP_024841182.1; and XM_005224667.4 and XP_005224724.1), mouse (NM_001003918.2 and NP_001003918.2; XM_006522138.3 and XP_006522201.1; XM_006522141.3 and XP_006522204.1; XM_006522139.3 and XP_006522202.1; and XM_030249116.1 and XP_030104976.1), rat (NM 001024790.1 and NP_001019961.1; XM_006245756.2 and XP_006245818.1; XM_006245758.3 and XP_006245820.1; XM_006245757.3 and XP_006245819.1; and XM_006245759.1 and XP_006245821.1); chicken (NM_001348012.1 and NP_001334941.1; NM_204471.2 and NP_989802.2; and XM_025155043.1 and XP_025010811.1), frog (XM_012970920.3 and XP_012826374.1; and XM_002939449.5 and XP_002939495.2), zebrafish (XM_005163957.3 and XP_005164014.1; XM_686123.9 and XP_691215.4; XM_021473871.1 and XP_021329546.1; XM_009299466.3 and XP_009297741.1; XM_009299464.3 and XP_009297739.2; and XM_009299465.3 and XP_009297740.2), and fruit fly (NM_132551.3 and NP_572779.2; and NM_001298220.1 and NP_001285149.1).

The term “USP7 activity” includes the ability of a USP7 polypeptide (and its fragments, domains, and/or motifs thereof, discussed herein) to bind its substrate and/or catalyze the ubiquitinase activity.

The term “USP7 inhibitor(s)” includes any natural or non-natural agent prepared, synthesized, manufactured, and/or purified by human that is capable of reducing, inhibiting, blocking, preventing, and/or that inhibits the ability of a USP7 polypeptide (and its fragments, domains, and/or motifs thereof, discussed herein). In one embodiment, such inhibitors may reduce or inhibit the binding/interaction between USP7 and its substrates or other binding partners. In still another embodiment, such inhibitors may increase or promote the turnover rate, reduce or inhibit the expression and/or the stability (e.g., the half-life), and/or change the cellular localization of USP7, resulting in at least a decrease in USP7 levels and/or activity. In yet another embodiment, such inhibitors may impair the catalytic activity of USP7. In still another embodiment, the inhibitors inhibit the deubiquitinase activity of USP7. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to USP7 or also inhibit at least one of the binding partners. Such inhibitors may include XL177A and/or XL188 (Shauer et al., Sci Rep 10, 5324 (2020)). Thus, in one embodiment, a USP7 inhibitor is XL177A, which has the following structure:

In another embodiment, the USP7 inhibitor is XL188, which has the following structure:

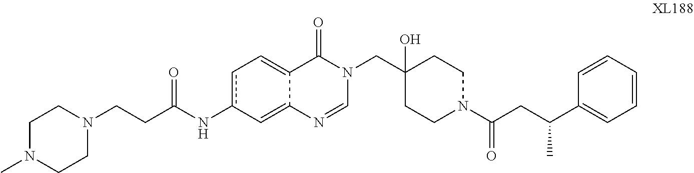

Such inhibitors may also include P-22077 (Cas No. 1247819-59-5). Additional USP7 inhibitors are known in the art, such as in PCT Publ. No. WO 2019/067503, U.S. Ser. No. 16/650,727, and PCT Publ. No. WO 2020/086595.

RNA interference for USP7 polypeptides are well known and commercially available (e.g., human, rat, or mouse shRNA/siRNA products (Cat. #TL308454V, TR308454, SR305301, TL308454, SR422076, TL308454V, TF308454, TL513496, SR513215, TR513496, TR702701, TL702701, TL702701V, and TL513496V from Origene (Rockville, Md.), and human or mouse gene knockout kit via CRISPR (Cat. #KN413986, KN518814, KN213986, KN318814, KN213986LP, KN213986RB, KN213986BN, KN318814LP, KN318814BN, and KN318814RB) from Origene (Rockville, Md.), and siRNA/shRNA products (Cat. #sc-41521 and sc-77373) from Santa Cruz Biotechonology (Dallas, Tex.). Methods for detection, purification, and/or inhibition of USP7 (e.g., by anti-USP7 antibodies) are also well known and commercially available (e.g., multiple USP7 antibodies from Signalway Antibody (College Park, Md., Cat. #38401, 27041, and 43178), Sino Biological (Wayne, Pa.; Cat. #11681-MM01), Invitrogen (Carlsbad, Calif., Cat. #Cat #PA5-17179, Cat #MA5-15585, etc.). USP7 knockout human cell lines are also well known and available at Horizon (Cambridge, UK, Cat. #HD 115-028, HDR02-029, and HDR02-028).

The term “MYCL,” also known as “MYCL proto-oncogene, bHLH transcription factor” refers to a bHLH protein and member of the polycomb repression complex (PRC) 1.1 that has DNA binding and transcription factor activity. Efficient DNA binding requires dimerization with another bHLH protein (e.g., MAX).

The term “MYCL” is intended to include fragments, variants (e.g., allelic variants) and derivatives thereof. The nucleic acid and amino acid sequences of a representative human MYCL is available to the public at the GenBank database (Gene ID 4610) and is shown in Table 1. Multiple transcript variants encoding several different isoforms have been found for MYCL. Human MYCL variants include the transcript variant 1 encoding isoform 1 (NM_001033081.3 and NP_001028253.1), the transcript variant 2 encoding isoform 2 (NM_005376.5 and NP_005367.2), and the transcript variant 3 encoding isoform 3 (NM_001033082.3 and NP_001028254.2).

Nucleic acid and polypeptide sequences of MYCL orthologs in organisms other than humans are well known and include, for example, chimpanzee (XM_016959814.2 and XP_016815303.2), Rhesus macaque (XM_028835497.1 and XP_028691330.1; and XM_015136019.2 and XP_014991505.2), dog (XM_022427768.1 and XP_022283476.1; XM_022427769.1 and XP_022283477.1; XM_022427775.1 and XP_022283483.1; XM_022427774.1 and XP_022283482.1; XM_022427778.1 and XP_022283486.1; XM_022427767.1 and XP_022283475.1; XM_022427772.1 and XP_022283480.1; XM_005628887.3 and XP_005628944.1; XM_022427777.1 and XP_022283485.1; XM_022427780.1 and XP_022283488.1; XM_022427771.1 and XP_022283479.1; XM_014119333.2 and XP_013974808.1; XM_022427779.1 and XP_022283487.1; XM_022427773.1 and XP_022283481.1; XM_022427776.1 and XP_022283484.1; XM_022427781.1 and XP_022283489.1; XM_005628888.3 and XP_005628945.1; and XM_539578.6 and XP_539578.2), cow (XM_005204928.4 and XP_005204985.1), mouse (NM_001303121.1 and NP_001290050.1; and NM_008506.3 and NP_032532.1), and rat (NM_001191763.1 and NP_001178692.1), chicken (XM_425790.6 and XP_425790.2), frog (NM_001011144.1 and NP_001011144.1), and zebrafish (NM_001045142.1 and NP_001038607.1).

The term “MYCL activity” includes the ability of a MYCL polypeptide (and its fragments, domains, and/or motifs thereof, discussed herein) to bind to DNA and/or activate transcription.

The term “MYCL-regulated pathway(s)” includes pathways in which MYCL (and its fragments, domains, and/or motifs thereof, discussed herein) binds to template DNA and activates transcription of at least one gene in the pathway. MYCL-regulated pathways include at least those described herein, such as regulation of expression of genes that suppress MHC class I, such as HLA I, surface expression in cancer cells.

The term “MYCL inhibitor(s)” includes any natural or non-natural agent prepared, synthesized, manufactured, and/or purified by human that is capable of reducing, inhibiting, blocking, preventing, and/or that inhibits the ability of a MYCL polypeptide (and its fragments, domains, and/or motifs thereof, discussed herein). In one embodiment, such inhibitors may reduce or inhibit the binding/interaction between MYCL and DNA or MYCL and its binding partners. In another embodiment, such inhibitors may reduce or inhibit MYCL as a transcription factor. In still another embodiment, such inhibitors may increase or promote the turnover rate, reduce or inhibit the expression and/or the stability (e.g., the half-life), and/or change the cellular localization of MYCL, resulting in at least a decrease in MYCL levels and/or activity. In yet another embodiment, such inhibitors may impair the catalytic activity of MCYL. In still another embodiment, the inhibitors inhibit the transcription activation activity of MYCL. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to MYCL or also inhibit at least one of the binding partners. RNA interference molecules for MYCL polypeptides are well known and commercially available (e.g., human, rat, or mouse shRNA/siRNA products (Cat. #TL311321V, SR303026, TL513612, SR412416, TR513612, TR311321, SR303026, TL311321, TL513612V, TL311321V, and TL316626V) from Origene, siRNA/shRNA products (Cat. #sc-38071) from Santa Cruz Biotechonology. Methods for detection, purification, and/or inhibition of MYCL (e.g., by anti-MYCL antibodies) are also well known and commercially available (e.g., multiple MYCL antibodies from Origene (Cat. #TA339110 and TA590604), Biorybt (Cambridge, UK; Cat. #orb324619 orb540520), Invitrogen (Cat. #PA1-30045, PA5-109998, etc.), abcam (Cambridge, Mass., Cat. #ab28739, ab167315, and others), etc.). MYCL knockout human cell lines are also well known and available at Horizon (Cat. #HZGHC4610).

The term “KDM2B,” also known as “Lysine Demethylase 2B” refers to histone demethylase that demethylates ‘Lys-4’ and ‘Lys-36’ of histone H3. KDM2B is a member of the F-box protein family, which is characterized by the “F-box,” an approximately 40 amino acid motif F-box proteins are a component of the ubiquitin protein ligase complex called SCF (SKP1-cullin-F-box). There are three classes of F-box proteins. Fbws F-box proteins comprise WD-40 domains, Fbls F-box proteins comprise containing leucine-rich repeats, and Fbxs F-box proteins comprise either different protein-protein interaction modules or no recognizable motifs. KDM2B belongs to the Fbls class. Alternative splicing results in multiple transcript variants.

The term “KDM2B” is intended to include fragments, variants (e.g., allelic variants) and derivatives thereof.

The nucleic acid and amino acid sequences of a representative human KDM2B is available to the public at the GenBank database (Gene ID 84678) and is shown in Table 1. Multiple transcript variants encoding several different isoforms have been found for KDM2B, including at least 5 different human transcript variants generated by alternative splicing (see World Wide Web at uniprot.org/uniprot/Q8NHM5). Human KDM2B variants include the transcript variant 1 encoding isoform b (NM_001005366.2 and NP_001005366.1), transcript variant 2 encoding isoform a (NM_032590.5 and NP_115979.3), transcript variant 3 encoding isoform X1 (XM_011538867.3 and XP_011537169.1), transcript variant 4 encoding isoform X2 (XM_011538868.3 and XP_011537170.1), transcript variant 5 encoding isoform X4 (XM_005253955.4 and XP_005254012.1), transcript variant 6 encoding isoform X5 (XM_005253956.4 and XP_005254013.1), transcript variant 7 encoding isoform X7 (XM_005253961.5 and XP_005254018.1), transcript variant 8 encoding isoform X6 (XM_011538875.3 and XP_011537177.1), and transcript variant 9 encoding isoform X3 (XM_011538869.2 and XP_011537171.1). Nucleic acid and polypeptide sequences of KDM2B orthologs in organisms other than humans are well known and include, for example, chimpanzee (XM_024348087.1 and XP_024203855.1; XM_024348082.1 and XP_024203850.1; XM_024348080.1 and XP_024203848.; XM_024348079.1 and XP_024203847.1; XM_009426426.3 and XP_009424701.1; XM_009426436.3 and XP_009424711.1; XM_024348085.1 and XP_024203853.1; XM_024348088.1 and XP_024203856.1; XM_024348090.1 and XP_024203858.1; XM_024348086.1 and XP_024203854.1; XM_024348083.1 and XP_024203851.1; XM_024348094.1 and XP_024203862.1; XM_024348089.1 and XP_024203857.1; XM_024348091.1 and XP_024203859.1; XM_024348092.1 and XP_024203860.1; XM_024348093.1 and XP_024203861.1; XM_009426440.3 and XP_009424715.1; XM_024348084.1 and XP_024203852.1; XM_001164996.5 and XP_001164996.1; XM_016924419.2 and XP_016779908.; XM_024348081.1 and XP_024203849.1; XM_009426429.3 and XP_009424704.1; and XM_009426431.3 and XP_009424706.1), rhesus macaque (XM_028830002.1 and XP_028685835.; XM_015152992.2 and XP_015008478.; XM_015152996.2 and XP_015008482.; XM_015152991.2 and XP_015008477.; XM_015153000.2 and XP_015008486.; XM_015152998.2 and XP_015008484.; XM_015152993.2 and XP_015008479.; XM_015152994.2 and XP_015008480.; XM_015152999.2 and XP_015008485.; XM_015152997.2 and XP_015008483.; XM_015152995.2 and XP_015008481.; XM_028830004.1 and XP_028685837.; XM_015153003.2 and XP_015008489.; XM_015153002.2 and XP_015008488.; XM_015153001.2 and XP_015008487.; and XM_028830003.1 and XP_028685836.1), dog (XM_005636193.3 and XP_005636250.; XM_022410683.1 and XP_022266391.; XM_022410682.1 and XP_022266390.; XM_005636186.3 and XP_005636243.; XM_005636191.3 and XP_005636248.; XM_005636187.3 and XP_005636244.; XM_022410687.1 and XP_022266395.; XM_005636188.3 and XP_005636245.; XM_005636189.3 and XP_005636246.; XM_022410688.1 and XP_022266396.; XM_005636192.1 and XP_005636249.; XM_022410686.1 and XP_022266394.; XM_022410685.1 and XP_022266393.; XM_022410689.1 and XP_022266397.; XM_005636194.3 and XP_005636251.; XM_005636195.1 and XP_005636252.; XM_005636197.3 and XP_005636254.1; and XM_005636196.2 and XP_005636253.2), cow (XM_010814030.3 and XP_010812332.; XM_005217980.4 and XP_005218037.; XM_005217983.4 and XP_005218040.; XM_024977708.1 and XP_024833476.; XM_024977709.1 and XP_024833477.; XM_005217982.2 and XP_005218039.; XM_005217985.2 and XP_005218042.; XM_024977704.1 and XP_024833472.; XM_024977705.1 and XP_024833473.; XM_024977706.1 and XP_024833474.; XM_024977707.1 and XP_024833475.; XM_024977711.1 and XP_024833479.; and XM_024977710.1 and XP_024833478.1), mouse (NM_001003953.2 and NP_001003953.; NM_001378863.1 and NP_001365792.1; NM_001378864.1 and NP_001365793.1; NM 001378865.1 and NP_001365794.1; NM_013910.2 and NP_038938.; XM_006530376.4 and XP_006530439.; XM_011248210.3 and XP_011246512.; XM_011248208.3 and XP_011246510.; XM_011248212.3 and XP_011246514.; XM_011248211.3 and XP_011246513.; XM_030254558.1 and XP_030110418.; XM_011248215.2 and XP_011246517.; XM_011248214.3 and XP_011246516.; XM_011248213.3 and XP_011246515.; XM_011248216.2 and XP_011246518.; XM_011248217.3 and XP_011246519.; and XM_030254560.1 and XP_030110420.1), rat (NM_001100679.1 and NP_001094149.1; and XM_017598337.1 and XP_017453826.1), chicken (XM_025155631.1 and XP_025011399.; XM_004945559.3 and XP_004945616.; XM_004945555.3 and XP_004945612.; XM_004945553.3 and XP_004945610.; XM_004945557.3 and XP_004945614.; XM_015275594.2 and XP_015131080.; XM_004945558.3 and XP_004945615.; XM_004945556.3 and XP_004945613.; XM_015275593.2 and XP_015131079.; XM_004945562.2 and XP_004945619.; XM_004945563.3 and XP_004945620.; and XM_004945561.1 and XP_004945618.1), and frog (XM_031892630.1 and XP_031748490.1; XM_031892631.1 and XP_031748491.1; XM_031892638.1 and XP_031748498.1; XM_031892646.1 and XP_031748506.1; and XM_031892655.1 and XP_031748515.1).

The term “KDM2B activity” includes the ability of a KDM2B polypeptide (and its fragments, domains, and/or motifs thereof, discussed herein) to bind its substrates, and/or mediate its demethylase activity.

The term “KDM2B substrate(s)” refers to binding partners of a KDM2B polypeptide (and its fragments, domains, and/or motifs thereof, discussed herein), e.g., the proteins listed herein, including SKP1 and a cullin protein. The term “KDM2B regulated pathway(s)” includes pathways in which KDM2B (and its fragments, domains, and/or motifs thereof, discussed herein) binds to at least one of its substrate, through which at least one cellular function and/or activity and/or cellular protein profiles is changed. KDM2B-regulated pathways include at least those described herein, such as positive or negative regulation of histone modification.

The term “agents that decrease the copy number, the expression level, and/or the activity of KDM2B,” or the term “agents that decrease the amount and/or activity of KDM2B” encompasses any natural or non-natural agent prepared, synthesized, manufactured, and/or purified by human that is capable of decreasing the expression level and/or activity of a KDM2B polypeptide (and its fragments, domains, and/or motifs thereof, discussed herein). In some embodiments, the agent may decrease the binding/interaction between KDM2B and its substrates or other binding partners. For example, the agent may increase the recognition and/or binding of KDM2B to histones thereby decreasing demethylation of the histones. In other embodiments, the agent may decrease the expression of a KDM2B polypeptide. In yet other embodiments, such agent may decrease KDM2B's activity in enhancing the immune response against tumors. In still other embodiments, such inhibitors may increase the turnover rate, decrease the expression and/or the stability (e.g., the half-life), and/or change the cellular localization of KDM2B, resulting in at least a decrease in KDM2B levels and/or activity. Such agents may be any molecule, including but not limited to small molecule compounds, antibodies or intrabodies, RNA interfering (RNAi) agents (including at least siRNAs, shRNAs, microRNAs (miRNAs), piwi, and other well-known agents), and gene constructs that inhibit endogenous production of KDM2B or its fragments inside cancer cells. Such agents may be specific to KDM2B or also to at least one of the binding partners, including but not limited to SCF or a cullin polypeptide. Antibodies for detection of KDM2B are commercially available (Cat. #AP08592PU-N AP51620PU-N (OriGene); ab234082, ab5199 (Abcam); ab234082 (Santa Cruz). RNA interference for KDM2B polypeptides are well known and commercially available (e.g., human, rat, or mouse shRNA/siRNA products (Cat. #TL313046V, SR325364, SR420035, SR325364, TL313046, TG313046, TF514017, TL313046V, TR313046, TR514017, TL514017V, TL514017 and human or mouse gene knockout kit via CRISPR (Cat. #KN413999, KN508731) from Origene (Rockville, Md.), and siRNA/shRNA products (Cat. #sc-75005 and sc-75006) from Santa Cruz Biotechonology (Dallas, Tex.). Methods for detection, purification, and/or inhibition of KDM2B (e.g., by anti-KDM2B antibodies) are also well known and commercially available (e.g., (Cat. #AP08592PU-N AP51620PU-N(OriGene); ab234082, ab5199 (Abcam); ab234082 (Santa Cruz). In addition, human KDM2B knockout cell line is commercially available from Horizon (Cambridge, UK, Cat. #HZGHC014730c012).

The term “BCORL1,” also known as “BCL6 corepressor like 1” refers to a transcriptional corepressor that is found tethered to promoter regions by DNA-binding proteins. BCORL1 can interact with several class II histone deacetylases to repress transcription. Alternative splicing results in multiple transcript variants. The term “BCORL1” is intended to include fragments, variants (e.g., allelic variants) and derivatives thereof.

The nucleic acid and amino acid sequences of a representative human BCORL1 is available to the public at the GenBank database (Gene ID 63035) and is shown in Table 1. Multiple transcript variants encoding several different isoforms have been found for BCORL1, including at least 3 different human transcript variants generated by alternative splicing (see World Wide Web at uniprot.org/uniprot/Q5H9F3). Human BCORL1 variants include the transcript variant 1 encoding isoform 1a (NM_001184772.3 and NP_001171701; NM_001379450.1 and NP_001366379; and NM_001379451.1 and NP_001366380.), transcript variant 2 encoding isoform 1 (NM_021946.5 and NP_068765.3), transcript variant 3 encoding isoform X1 (XM_005262453.4 and XP_005262510.1; XM_006724777.3 and XP_006724840.1; XM_017029721.1 and XP_016885210.1; XM_006724776.3 and XP_006724839.1; XM_005262455.4 and XP_005262512.2; and XM_017029722.1 and XP_016885211.1), transcript variant 4 encoding isoform X3 (XM_005262456.4 and XP_005262513.2), and transcript variant 4 encoding isoform X2 (XM_006724779.2 and XP_006724842.1).

Nucleic acid and polypeptide sequences of BCORL1 orthologs in organisms other than humans are well known and include, for example, chimpanzee (XM_016943863.2 and XP_016799352.1; XM_016943867.1 and XP_016799356.1; XM_016943861.1 and XP_016799350.1; XM_016943870.1 and XP_016799359.; XM_016943862.1 and XP_016799351.1; XM_024353327.1 and XP_024209095.1; XM_016943864.2 and XP_016799353.1; XM_016943868.2 and XP_016799357.1; XM_016943866.2 and XP_016799355.1; and XM_016943865.1 and XP_016799354.1), rhesus macaque (XM_028842487.1 and XP_028698320.; XM_028842482.1 and XP_028698315.; XM_028842486.1 and XP_028698319.; XM_028842484.1 and XP_028698317.; XM_028842483.1 and XP_028698316.; XM_015128181.2 and XP_014983667.2; XM_015128183.2 and XP_014983669.2; and XM_028842485.1 and XP_028698318.1), dog (XM_005641794.3 and XP_005641851.1; XM_022416325.1 and XP_022272033.1; XM_538169.6 and XP_538169.3; and XM_005641793.3 and XP_005641850.1), cow (XM_005227504.4 and XP_005227561.1; XM_005227505.4 and XP_005227562.1; and XM_002699518.5 and XP_002699564.2), mouse (NM_178782.4 and NP_848897.3), rat (NM_001191587.1 and NP_001178516.1), chicken (XM_015278363.2 and XP_015133849.1; XM_015278362.2 and XP_015133848.1; and XM_025150323.1 and XP_025006091.1), and frog NM_001142070.1 and NP_001135542.1; XM_012968111.3 and XP_012823565.1; XM_018096174.2 and XP_017951663.1; XM_012968109.3 and XP_012823563.1; and XM_012968112.3 and XP_012823566.1

The term “BCORL1 activity” includes the ability of a BCORL1 polypeptide (and its fragments, domains, and/or motifs thereof, discussed herein) to bind its substrates, and/or mediate its transcription repression activity.

The term “BCORL1 substrate(s)” refers to binding partners of a BCORL1 polypeptide (and its fragments, domains, and/or motifs thereof, discussed herein.

The term “BCORL1 regulated pathway(s)” includes pathways in which BCORL1 (and its fragments, domains, and/or motifs thereof, discussed herein) binds to at least one of its substrate, through which at least one cellular function and/or activity and/or cellular protein profiles is changed. BCORL1-regulated pathways include at least those described herein, such as transcription regulation.

The term “agents that decrease the copy number, the expression level, and/or the activity of BCORL1,” or the term “agents that decrease the amount and/or activity of BCORL1” encompasses any natural or non-natural agent prepared, synthesized, manufactured, and/or purified by human that is capable of decreasing the expression level and/or activity of a BCORL1 polypeptide (and its fragments, domains, and/or motifs thereof, discussed herein). In some embodiments, the agent may decrease the binding/interaction between BCORL1 and its substrates or other binding partners. In other embodiments, the agent may decrease the expression of a BCORL1 polypeptide. In yet other embodiments, such agent may decrease BCORL1's activity in enhancing the immune response against tumors. In still other embodiments, such inhibitors may increase the turnover rate, decrease the expression and/or the stability (e.g., the half-life), and/or change the cellular localization of BCORL1, resulting in at least a decrease in BCORL1 levels and/or activity. Such agents may be any molecule, including but not limited to small molecule compounds, antibodies or intrabodies, RNA interfering (RNAi) agents (including at least siRNAs, shRNAs, microRNAs (miRNAs), piwi, and other well-known agents), and gene constructs that inhibit endogenous production of BCORL1 or its fragments inside cancer cells. Such agents may be specific to BCORL1 or also to at least one of its binding partners. RNA interference for BCORL1 polypeptides are well known and commercially available (e.g., human, rat, or mouse shRNA/siRNA products (Cat. #TL306414V, TF306414, TR519839, TR306414, SR311867, TL306414, SR423201) and human or mouse gene knockout kit via CRISPR (Cat. #KN419297, KN502121) from Origene (Rockville, Md.), and siRNA/shRNA products (Cat. #sc-141680) from Santa Cruz Biotechonology (Dallas, Tex.). Methods for detection, purification, and/or inhibition of BCORL1 (e.g., by anti-BCORL1 antibodies) are also well known and commercially available (Cat. #ab251816), ab251817) (Abcam). (BCORL1 knockout human cell lines are also well known and available at Horizon (Cambridge, UK, Cat. #HZGHC630358).

The term “RING1A,” also known as “ring finger protein 1” refers to a gene or protein belonging to the RING family. Ring family members are characterized by having a RING domain, a zinc-binding motif related to the zinc finger domain. RING1A interacts with polycomb group complex proteins BMI, EDR1, and CBX4. Alternative splicing results in multiple transcript variants. The term “RING1A” is intended to include fragments, variants (e.g., allelic variants) and derivatives thereof.

The nucleic acid and amino acid sequences of a representative human RING1A is available to the public at the GenBank database (Gene ID 6015) and is shown in Table 1. Multiple transcript variants encoding several different isoforms have been found for RING1A, including at least 2 different human transcript variants generated by alternative splicing (see World Wide Web at uniprot.org/uniprot/Q06587). Human RING1A variants include the transcript variant 1 encoding isoform 1 (NM_002931.4 and NP_002922.2).

Nucleic acid and polypeptide sequences of RING1A orthologs in organisms other than humans are well known and include, for example, chimpanzee (NM_001081482.1 and NP_001074951.1; XM_009450849.3 and XP_009449124.1; and XM_016954658.2 and XP_016810147.1), rhesus macaque (NM_001114959.1 and NP_001108431.1; XM_028846856.1 and XP_028702689.1; and XM_015136067.2 and XP_014991553.1), dog (NM_001048128.1 and NP_001041593.1), cow (NM_001105051.1 and NP_001098521.1), mouse (NM_009066.3 and NP_033092.3), rat (NM_212549.2 and NP_997714.2; XM_017601640.1 and XP_017457129.1), and frog (NM_001097325.1 and NP_001090794.1).

The term “RING1A activity” includes the ability of a RING1A polypeptide (and its fragments, domains, and/or motifs thereof, discussed herein) to bind its substrates, and/or mediate its transcription repression activity.

The term “RING1A substrate(s)” refers to binding partners of a RING1A polypeptide (and its fragments, domains, and/or motifs thereof, discussed herein), e.g., the proteins listed herein, including BMI1, EDR1, and CBX4.

The term “RING1A regulated pathway(s)” includes pathways in which RING1A (and its fragments, domains, and/or motifs thereof, discussed herein) binds to at least one of its substrate, through which at least one cellular function and/or activity and/or cellular protein profiles is changed. RING1A-regulated pathways include at least those described herein, such as transcription repression.

The term “agents that decrease the copy number, the expression level, and/or the activity of RING1A,” or the term “agents that decrease the amount and/or activity of RING1A” encompasses any natural or non-natural agent prepared, synthesized, manufactured, and/or purified by human that is capable of decreasing the expression level and/or activity of a RING1A polypeptide (and its fragments, domains, and/or motifs thereof, discussed herein). In some embodiments, the agent may decrease the binding/interaction between RING1A and its substrates or other binding partners. In other embodiments, the agent may decrease the expression of a RING1A polypeptide. In yet other embodiments, such agent may decrease RING1A's activity in enhancing the immune response against tumors. In still other embodiments, such inhibitors may increase the turnover rate, decrease the expression and/or the stability (e.g., the half-life), and/or change the cellular localization of RING1A, resulting in at least a decrease in RING1A levels and/or activity. Such agents may be any molecule, including but not limited to small molecule compounds, antibodies or intrabodies, RNA interfering (RNAi) agents (including at least siRNAs, shRNAs, microRNAs (miRNAs), piwi, and other well-known agents), and gene constructs that inhibit endogenous production of RING1A or its fragments inside cancer cells. Such agents may be specific to RING1A or also to at least one of the binding partners, including but not limited to BMI1, EDR1, and CBX4. RNA interference for RING1A polypeptides are well known and commercially available (e.g., human, rat, or mouse shRNA/siRNA products (Cat. TL309810V, SR304071, SR304071, SR304082, TG309787, TG512489, TL309787, among others, and human or mouse gene knockout kit via CRISPR (Cat. KN514834, KN402650) from Origene (Rockville, Md.), and siRNA/shRNA products (Cat. #sc-38198, sc-77379, sc-106751, sc-38197, sc-62946, sc-62947) from Santa Cruz Biotechonology (Dallas, Tex.). Methods for detection, purification, and/or inhibition of RING1A (e.g., by anti-RING1A antibodies) are also well known and commercially available (e.g., (Cat. #C48439 (Signalway Antibody), CF809239 CF809256 (OriGene); ab175149, ab180170, ab32644, among others (Abcam); sc-517221 (Santa Cruz). In addition, human RING1A knockout cell line is commercially available from Horizon (Cambridge, UK, Cat. #HZGHC001111c003, HZGHC001111c012, and HZGHC001111cc001).

The term “RING1B,” also known as “ring finger protein 2” refers to a member of polycomb group complexes (e.g., PRC1.1) encoded by the RNF2 gene. RING1B has been shown to interact with and inhibit CP2, a transcription factor. RING1B also interacts with huntingtin interacting protein 2 (HIP2), a ubiquitin-conjugating enzyme and possesses ubiquitin ligase activity. The protein has chromatin binding and ubiquitin-protein transferase. The term “RING1B” is intended to include fragments, variants (e.g., allelic variants) and derivatives thereof.

The nucleic acid and amino acid sequences of a representative human RING1B is available to the public at the GenBank database (Gene ID 6045) and is shown in Table 1. Multiple transcript variants encoding several different isoforms have been found for RING1B, including at least 2 different human transcript variants generated by alternative splicing (see World Wide Web at uniprot.org/uniprot/Q99496). Human RING1B encodes the canonical sequence (NM_007212.4 and NP_009143.1). Human RING1B variants also include the transcript variant encoding isoform X1 (XM_011509852.2 and XP_011508154.1; and XM_011509851.3 and XP_011508153.1) and the transcript variant encoding isoform X2 (XM_005245413.3 and XP_005245470.1). Nucleic acid and polypeptide sequences of RING1B orthologs in organisms other than humans are well known and include, for example, chimpanzee (XM_514057.6 and XP_514057.3; XM_003308638.4 and XP_003308686.1; XM_009439605.3 and XP_009437880.1; and XM_009439610.3 and XP_009437885.1), dog (XM_022420969.1 and XP_022276677.1), cow (NM_001101203.1 and NP_001094673.1; XM_024976397.1 and XP_024832165.1; and XM_024976398.1 and XP_024832166.1), mouse (NM_001360844.1 and NP_001347773.1; NM_001360845.1 and NP_001347774.1; NM_001360847.1 and NP_001347776.1; and NM_011277.3 and NP_035407.1), rat (NM_001025667.1 and NP_001020838.1; XM_006249991.3 and XP_006250053.1; and XM_006249990.3 and XP_006250052.1), chicken (XM_015290550.2 and XP_015146036.1; and XM_015290551.2 and XP_015146037.1), frog (NM_213707.2 and NP_998872.1), zebrafish (NM_131213.2 and NP_571288.2); fruit fly (NM_058161.4 and NP_477509.1), and mosquito (XM_320974.5 and XP_320974.3).

The term “RING1B activity” includes the ability of a RING1B polypeptide (and its fragments, domains, and/or motifs thereof, discussed herein) to bind its substrates, and/or mediate its ubiquitin ligase activity.

The term “RING1B substrate(s)” refers to binding partners of a RING1B polypeptide (and its fragments, domains, and/or motifs thereof, discussed herein), e.g., the proteins listed herein, including C2 and HIP2.

The term “RING1B regulated pathway(s)” includes pathways in which RING1B (and its fragments, domains, and/or motifs thereof, discussed herein) binds to at least one of its substrate, through which at least one cellular function and/or activity and/or cellular protein profiles is changed. RING1B-regulated pathways include at least those described herein, such as development and cell proliferation.

The term “agents that decrease the copy number, the expression level, and/or the activity of RING1B,” or the term “agents that decrease the amount and/or activity of RING1B” encompasses any natural or non-natural agent prepared, synthesized, manufactured, and/or purified by human that is capable of decreasing the expression level and/or activity of a RING1B polypeptide (and its fragments, domains, and/or motifs thereof, discussed herein). In some embodiments, the agent may decrease the binding/interaction between RING1B and its substrates or other binding partners. In other embodiments, the agent may decrease the expression of a RING1B polypeptide. In yet other embodiments, such agent may decrease RING1B's activity in enhancing the immune response against tumors. In still other embodiments, such inhibitors may increase the turnover rate, decrease the expression and/or the stability (e.g., the half-life), and/or change the cellular localization of RING1B, resulting in at least a decrease in RING1B levels and/or activity. Such agents may be any molecule, including but not limited to small molecule compounds, antibodies or intrabodies, RNA interfering (RNAi) agents (including at least siRNAs, shRNAs, microRNAs (miRNAs), piwi, and other well-known agents), and gene constructs that inhibit endogenous production of RING1B or its fragments inside cancer cells. Such agents may be specific to RING1B or also to at least one of the binding partners, including but not limited to C2 and HIP2. Antibodies for detection of RING1B are commercially available (Cat. #R1502P TA302592 (OriGene); ab187509, ab181140, ab101273, among others (Abcam); sc-101109 (Santa Cruz). RNA interference for RING1B polypeptides are well known and commercially available (e.g., human, rat, or mouse shRNA/siRNA products (Cat. #TL309787V, SR304082, SR304082, TG309787, TG512489, among others, and human or mouse gene knockout kit via CRISPR (Cat. #KN514934, KN403089) from Origene (Rockville, Md.), and siRNA/shRNA products (Cat. #sc-62946, sc-62947) from Santa Cruz Biotechonology (Dallas, Tex.). Methods for detection, purification, and/or inhibition of RING1B (e.g., by anti-RING1B antibodies) are also well known and commercially available (e.g., (Cat. #C49790 (Signalway Antibody; ABIN2781368, ABIN6207349 (antibodies-online.com, Limerick, Pa.). RING1B knockout human cell lines are also well known and available at Horizon (Cambridge, UK, Cat. #HZGHC001181c002, HZGHC001181c007, HZGHC001181c005, HZGHC001181c001, HZGHC001181c003, among others).

The term “RYBP,” also known as “RING1 And YY1 Binding Protein” refers to a member of the polycomb repressive complex 1 (and 1.1). RYBP is a transcription corepressor. Alternative splicing results in multiple transcript variants. The term “RYBP” is intended to include fragments, variants (e.g., allelic variants) and derivatives thereof.

The nucleic acid and amino acid sequences of a representative human RYBP is available to the public at the GenBank database (Gene ID 23429) and is shown in Table 1. A single transcript variant encoding RYBP has been identified (see World Wide Web at uniprot.org/uniprot/Q8N488; NM_001005366.2 and NP_001005366.1).

Nucleic acid and polypeptide sequences of RYBP orthologs in organisms other than humans are well known and include, for example, chimpanzee (XM_016941488.2 and XP_016796977.1), dog (XM_022407339.1 and XP_022263047.1), mouse (NM_019743.3 and NP_062717.2), and chicken (XM_015293232.2 and XP_015148718.1). RYBP The term “RYBP substrate(s)” refers to binding partners of a RYBP polypeptide (and its fragments, domains, and/or motifs thereof, discussed herein.

The term “RYBP-regulated pathway(s)” includes pathways in which RYBP (and its fragments, domains, and/or motifs thereof, discussed herein) binds to at least one of its substrate, through which at least one cellular function and/or activity and/or cellular protein profiles is changed. RYBP regulated pathways include at least those described herein, such as the E2F transcription factor network and chromatin regulation and acetylation.

The term “agents that decrease the copy number, the expression level, and/or the activity of RYBP,” or the term “agents that decrease the amount and/or activity of RYBP” encompasses any natural or non-natural agent prepared, synthesized, manufactured, and/or purified by human that is capable of decreasing the expression level and/or activity of a RYBP polypeptide (and its fragments, domains, and/or motifs thereof, discussed herein). In some embodiments, the agent may decrease the binding/interaction between RYBP and its substrates or other binding partners. In other embodiments, the agent may decrease the expression of a RYBP polypeptide. In yet other embodiments, such agent may decrease RYBP activity in enhancing the immune response against tumors. In still other embodiments, such inhibitors may increase the turnover rate, decrease the expression and/or the stability (e.g., the half-life), and/or change the cellular localization of RYBP, resulting in at least a decrease in RYBP levels and/or activity. Such agents may be any molecule, including but not limited to small molecule compounds, antibodies or intrabodies, RNA interfering (RNAi) agents (including at least siRNAs, shRNAs, microRNAs (miRNAs), piwi, and other well-known agents), and gene constructs that inhibit endogenous production of RYBP or its fragments inside cancer cells. Such agents may be specific to RYBP or also to at least one of the binding partners. RNA interference for RYBP polypeptides are well known and commercially available (e.g., human, rat, or mouse shRNA/siRNA products (Cat. #TL309675V, SR308270, TL503156, SR308270, TR309675, R404933, among others and human or mouse gene knockout kit via CRISPR (Cat. #KN406186, KN515228) from Origene (Rockville, Md.), and siRNA/shRNA products (Cat. #sc-77379, sc-106751) from Santa Cruz Biotechonology (Dallas, Tex.). Methods for detection, purification, and/or inhibition of RYBP (e.g., by anti-RYBP antibodies) are also well known and commercially available (e.g., (Cat. #28645 (Signalway Antibodies); ABIN1156059, ABIN1156058 (antibodies-online.com); RYBP (A-1), RYBP (A-1) X (Santa Cruz). Antibodies that specifically bind RYBP are commercially available (Cat. #AP00095PU-N, AP07729PU-N (OriGene); ab185971, ab250871, ab5976, ab107896, ab89603 (Abcam); RYBP (A-1), RYBP (A-1) X (Santa Cruz). RYBP knockout human cell lines are also well known and available at Horizon (Cambridge, UK, Cat. #HZGHC23429).

The term “PCGF1,” also known as “Polycomb Group Ring Finger 1” refers to a member of the PRC1.1 complex. An important paralog of this gene is COMMD3-BMI1. Alternative splicing results in multiple transcript variants. The term “PCGF1” is intended to include fragments, variants (e.g., allelic variants) and derivatives thereof.

The nucleic acid and amino acid sequences of a representative human PCGF1 is available to the public at the GenBank database (Gene ID 84759) and is shown in Table 1. Multiple transcript variants encoding several different isoforms have been found for PCGF1, including at least 2 different human transcript variants generated by alternative splicing (see World Wide Web at uniprot.org/uniprot/Q9BSM1). Human PCGF1 variants include transcript variant 1 encoding isoform 1 (NM_032673.3 and NP_116062.2) and transcript variant 2 encoding isoform X1 (XM_024453181.1 and XP_024308949.1).

Nucleic acid and polypeptide sequences of PCGF1 orthologs in organisms other than humans are well known and include, for example, chimpanzee (XM_515562.6 and XP_515562.2), rhesus macaque (XM_015112696.2 and XP_014968182.1), dog (XM_022404797.1 and XP_022260505.1; XM_005630527.3 and XP_005630584.1; XM_005630524.3 and XP_005630581.1; XM_005630526.3 and XP_005630583.1; XM_005630529.3 and XP_005630586.1; XM_532995.6 and XP_532995.2; and XM_022404796.1 and XP_022260504.1), cow (NM_001046447.2 and NP_001039912.2), mouse (XM_030255588.1 and XP_030111448.1, rat (NM_001007000.1 and NP_001007001.1), chicken (XM_015273146.2 and XP_015128632.1), zebrafish (NM_001007158.2 and NP_001007159.1; and XM_009307695.3 and XP_009305970.1). The term “PCGF1 activity” includes the ability of a PCGF1 polypeptide (and its fragments, domains, and/or motifs thereof, discussed herein) to bind its substrates, and/or mediate its activity.

The term “PCGF1 substrate(s)” refers to binding partners of a PCGF1 polypeptide (and its fragments, domains, and/or motifs thereof, discussed herein.

The term “PCGF1 regulated pathway(s)” includes pathways in which PCGF1 (and its fragments, domains, and/or motifs thereof, discussed herein) binds to at least one of its substrate, through which at least one cellular function and/or activity and/or cellular protein profiles is changed.

The term “agents that decrease the copy number, the expression level, and/or the activity of PCGF1,” or the term “agents that decrease the amount and/or activity of PCGF1” encompasses any natural or non-natural agent prepared, synthesized, manufactured, and/or purified by human that is capable of decreasing the expression level and/or activity of a PCGF1 polypeptide (and its fragments, domains, and/or motifs thereof, discussed herein). In some embodiments, the agent may decrease the binding/interaction between PCGF1 and its substrates or other binding partners. In other embodiments, the agent may decrease the expression of a PCGF1 polypeptide. In yet other embodiments, such agent may decrease PCGF1's activity in enhancing the immune response against tumors. In still other embodiments, such inhibitors may increase the turnover rate, decrease the expression and/or the stability (e.g., the half-life), and/or change the cellular localization of PCGF1, resulting in at least a decrease in PCGF1 levels and/or activity. Such agents may be any molecule, including but not limited to small molecule compounds, antibodies or intrabodies, RNA interfering (RNAi) agents (including at least siRNAs, shRNAs, microRNAs (miRNAs), piwi, and other well-known agents), and gene constructs that inhibit endogenous production of PCGF1 or its fragments inside cancer cells. Such agents may be specific to PCGF1 or also to at least one of the binding partners. Antibodies for detection of PCGF1 are commercially available (Cat. #TA330488 (OriGene); ab84108, ab194556) (Abcam); sc-515371 (Santa Cruz). RNA interference for PCGF1 polypeptides are well known and commercially available (e.g., human, rat, or mouse shRNA/siRNA products (Cat. #TL302590V, SR313658, TR302590, SR406929, SR313658, TL302590 among others) and human or mouse gene knockout kit via CRISPR (Cat. #KN512948, KN416322) from Origene (Rockville, Md.), and siRNA/shRNA products (Cat. #sc-152107, sc-94353) from Santa Cruz Biotechonology (Dallas, Tex.). Methods for detection, purification, and/or inhibition of PCGF1 (e.g., by anti-PCGF1 antibodies) are also well known and commercially available (e.g., (Cat. #BIN6208970, ABIN6208971 (antibodies-online.com); 30713, C30713 (Signalway Antibody. PCGF1 knockout human cell lines are also well known and available at Horizon (Cambridge, UK, Cat. #HZGHC84759).

The term “SKP1,” also known as “S-phase kinase-associated protein 1” refers to a protein that is a component of SCF complexes, which are involved in the ubiquitination of protein substrates. These complexes are described supra. Alternative splicing results in multiple transcript variants. The term “SKP1” is intended to include fragments, variants (e.g., allelic variants) and derivatives thereof.

The nucleic acid and amino acid sequences of a representative human SKP1 is available to the public at the GenBank database (Gene ID 6500) and is shown in Table 1. Multiple transcript variants encoding several different isoforms have been found for SKP1, including at least 2 different human transcript variants generated by alternative splicing (see World Wide Web at uniprot.org/uniprot/P63208). Human SKP1 variants include transcript variant 1 encoding isoform a (NM_006930.3 and NP_008861.2) and transcript variant 2 encoding isoform b (NM_170679.3 and NP_733779.1).

Nucleic acid and polypeptide sequences of SKP1 orthologs in organisms other than humans are well known and include, for example, chimpanzee (XM_001166401.6 and XP_001166401.1), dog (NM_001252408.1 and NP_001239337.1), cow (NM_001034781.2 and NP_001029953.1), mouse (NM_011543.4 and NP_035673.3; and XM_006532786.2 and XP_006532849.1), rat (NM_001007608.2 and NP_001007609.1), chicken (NM_001006153.1 and NP_001006153.1; XM_025154856.1 and XP_025010624.1; XM_025154857.1 and XP_025010625.1), frog (NM_001016519.3 and NP_001016519.1; XM_012959026.3 and XP_012814480.1), fruit fly (NM_166858.3 and NP_726692.1; NM_058042.5 and NP_477390.1; NM_001038729.3 and NP_001033818.1; NM_166857.3 and NP_726691.1; NM_166856.3 and NP_726690.1; NM_166861.3 and NP_726695.1; NM_166860.3 and NP_726694.1; NM_166859.3 and NP_726693.1; NM_001297826.1 and NP_001284755.1). The term “SKP1 activity” includes the ability of a SKP1polypeptide (and its fragments, domains, and/or motifs thereof, discussed herein) to bind its substrates, which as a SCF complex is involved in cell cycle progression, signal transduction and transcription.

The term “SKP1 substrate(s)” refers to binding partners of a SKP1 polypeptide (and its fragments, domains, and/or motifs thereof, discussed herein), e.g., the proteins listed herein, including Cul1 and F-box proteins.

The term “SKP1-regulated pathway(s)” includes pathways in which SKP1 (and its fragments, domains, and/or motifs thereof, discussed herein) binds to at least one of its substrate, through which at least one cellular function and/or activity and/or cellular protein profiles is changed. SKP1-regulated pathways include at least those described herein.

The term “agents that decrease the copy number, the expression level, and/or the activity of SKP1,” or the term “agents that decrease the amount and/or activity of SKP1” encompasses any natural or non-natural agent prepared, synthesized, manufactured, and/or purified by human that is capable of decreasing the expression level and/or activity of a SKP1 polypeptide (and its fragments, domains, and/or motifs thereof, discussed herein). In some embodiments, the agent may decrease the binding/interaction between SKP1 and its substrates or other binding partners. In other embodiments, the agent may decrease the expression of a SKP1 polypeptide. In yet other embodiments, such agent may decrease SKP1 activity in enhancing the immune response against tumors. In still other embodiments, such inhibitors may increase the turnover rate, decrease the expression and/or the stability (e.g., the half-life), and/or change the cellular localization of SKP1, resulting in at least a decrease in SKP1 levels and/or activity. Such agents may be any molecule, including but not limited to small molecule compounds, antibodies or intrabodies, RNA interfering (RNAi) agents (including at least siRNAs, shRNAs, microRNAs (miRNAs), piwi, and other well-known agents), and gene constructs that inhibit endogenous production of SKP1 or its fragments inside cancer cells. Such agents may be specific to SKP1 or also to at least one of the binding partners, including but not limited to F-box proteins and cullin (e.g., CUL1). Antibodies for detection of SKP1 are commercially available (Cat. #AM06704SU-N, AM06720SU-N(OriGene); ab76502, ab233484, ab228637 (Abcam); sc-136301, sc-5281 (Santa Cruz). RNA interference for SKP1 polypeptides are well known and commercially available (e.g., human, rat, or mouse shRNA/siRNA products (Cat. #TL301685V, SR304387, TR301685, SR304387, TF502226, TR502226 and human or mouse gene knockout kit via CRISPR (Cat. #KN406509) from Origene (Rockville, Md.), and siRNA/shRNA products (Cat. #sc-29482, sc-153916, sc-36498, sc-76605) from Santa Cruz Biotechonology (Dallas, Tex.). Methods for detection, purification, and/or inhibition of SKP1 (e.g., by anti-SKP1 antibodies) are also well known and commercially available (e.g., (Cat. #ABIN822770 ABIN421487 (antibodies-online.com); EK7324, EK16636 (Signalway Antibody.

The term “BCOR,” also known as “BCL6 Corepressor” refers to a corepressor that interacts with POZ domain of BCL6. BCOR is also known to interact with classes of histone deacetylases. Alternative splicing results in multiple transcript variants. The term “BCOR” is intended to include fragments, variants (e.g., allelic variants) and derivatives thereof.

The nucleic acid and amino acid sequences of a representative human BCOR is available to the public at the GenBank database (Gene ID 54880) and is shown in Table 1. Multiple transcript variants encoding several different isoforms have been found for BCOR, including at least four different human transcript variants generated by alternative splicing (see World Wide Web at uniprot.org/uniprot/Q6W2J9). Human BCOR variants include the transcript variant 3 encoding isoform a (NM_001123383.1 and NP_001116855.1), transcript variant 4 encoding isoform b (NM_001123384.2 and NP_001116856.1), transcript variant 5 encoding isoform c (NM_001123385.2 and NP_001116857), transcript variant 1 encoding isoform a (NM_017745.6 and NP_060215.4), transcript variant X1 encoding isoform X1 (XM_005272616.1 and XP_005272673.1), transcript variant X6 encoding isoform X1 (XM_011543931.2 and XP_011542233.1), transcript variant X5 encoding isoform X1 (XM_011543930.1 and XP_011542232.1), transcript variant X2 encoding isoform X1 (XM_011543929.2 and XP_011542231.1), transcript variant X4 encoding isoform X1 (XM_005272618.3 and XP_005272675.1), transcript variant X8 encoding isoform X3 (XM_017029615.1 and XP_016885104.1), transcript variant X3 encoding isoform X1 (XM_006724536.3 and XP_006724599.1), transcript variant X9 encoding isoform X4 (XM_005272620.4 and XP_005272677.1), transcript variant X7 encoding isoform X2 (XM_005272619.4 and XP_005272676.1), transcript variant X10 encoding isoform X5 (XM_017029616.2 and XP_016885105.1), Nucleic acid and polypeptide sequences of BCOR orthologs in organisms other than humans are well known and include, for example, chimpanzee (XM_016943431.2 and XP_016798920.1; XM_016947534.2 and XP_016803023.1; XM_016947536.2 and XP_016803025.1; XM_016943432.1 and XP_016798921.1; XM_016943433.1 and XP_016798922.1; XM_016943436.1 and XP_016798925.1; XM_016943430.1 and XP_016798919.1; XM_016943437.1 and XP_016798926.1; and XM_016943435.1 and XP_016798924.1; XM_016943434.2 and XP_016798923.1.), Rhesus monkey (XM_015127207.2 and XP_014982693.2; XM_015127203.2 and XP_014982689.2; XM_028842387.1 and XP_028698220.1; XM_028842388.1 and XP_028698221.1; XM_028842389.1 and XP_028698222.1; XM_015127204.2 and XP_014982690.2; XM_015127202.2 and XP_014982688.2; XM_015127208.2 and XP_014982694.2; XM_015127206.2 and XP_014982692.2; XM_015127212.2 and XP_014982698.2; XM_015127210.2 and XP_014982696.2; XM_015127205.2 and XP_014982691.2; XM_015127211.2 and XP_014982697.2; XM_015127209.2 and XP_014982695.2), dog (XM_537997.6 and XP_537997.2; XM_022415576.1 and XP_022271284.1; XM_022415575.1 and XP_022271283.1; XM_005641249.3 and XP_005641306.1; XM_005641250.3 and XP_005641307.1; XM_855998.5 and XP_861091.1; XM_005641247.3 and XP_005641304.1; XM_855945.5 and XP_861038.1; XM_005641248.3 and XP_005641305.1), cow (NM_001191544.3 and NP_001178473.3; XM_024988315.1 and XP_024844083.1; XM_005228295.4 and XP_005228352.2; XM_005228296.4 and XP_005228353.2; XM_024988316.1 and XP_024844084.1; XM_024988314.1 and XP_024844082.1; XM_024988322.1 and XP_024844090.1; XM_024988319.1 and XP_024844087.1; XM_024988323.1 and XP_024844091.1; XM_024988320.1 and XP_024844088.1; XM_024988324.1 and XP_024844092.1; XM_024988325.1 and XP_024844093.1; XM_024988317.1 and XP_024844085.1; XM_024988318.1 and XP_024844086.1), mouse (NM_001168321.1 and NP_001161793.1; NM_029510.3 and NP_083786.2; NM_175044.3 and NP_778209.2; NM_175045.3 and NP_778210.2; NM_175046.3 and NP_778211.2; XM_017318623.2 and XP_017174112.1; XM_017318621.2 and XP_017174110.1; XM_030251502.1 and XP_030107362.1; XM_017318622.2 and XP_017174111.1; XM_030251503.1 and XP_030107363.1; XM_030251500.1 and XP_030107360.1; XM_030251501.1 and XP_030107361.1; XM_030251499.1 and XP_030107359.1; XM_017318624.2 and XP_017174113.1), rat (NM_001191586.1 and NP_001178515.1; XM_006256660.3 and XP_006256722.1; XM_006256659.3 and XP_006256721.1; XM_006256664.3 and XP_006256726.1; XM_006256663.3 and XP_006256725.1; XM_006256665.3 and XP_006256727.1; XM_006256661.3 and XP_006256723.1), chicken (XM_025146057.1 and XP_025001825.1; XM_025146076.1 and XP_025001844.1; XM_025146086.1 and XP_025001854.1; XM_025146070.1 and XP_025001838.1; XM_025146082.1 and XP_025001850.1; XM_025146092.1 and XP_025001860.1; XM_025146062.1 and XP_025001830.1; XM_015302080.2 and XP_015157566.2), zebrafish (XM_005173943.4→XP_005174000.1), and frog (NM_001126679.1 and NP_001120151.1; XM_012956453.3 and XP_012811907.1; XM_012956456.3 and XP_012811910.1; XM_012956450.3 and XP_012811904.1; XM_012956452.3 and XP_012811906.1; XM_018091086.1 and XP_017946575.1; XM_031895681.1 and XP_031751541.1).

The term “BCOR activity” includes the ability of a BCOR polypeptide (and its fragments, domains, and/or motifs thereof, discussed herein) to bind its substrates, and/or mediate its corepressor activity.

The term “BCOR substrate(s)” refers to binding partners of a BCOR polypeptide (and its fragments, domains, and/or motifs thereof, discussed herein), e.g., the proteins listed herein, including BCL6.

The term “BCOR regulated pathway(s)” includes pathways in which BCOR (and its fragments, domains, and/or motifs thereof, discussed herein) binds to at least one of its substrate, through which at least one cellular function and/or activity and/or cellular protein profiles is changed. BCOR-regulated pathways include at least those described herein, such as positive or negative regulation of histone modification.

The term “agents that decrease the copy number, the expression level, and/or the activity of BCOR,” or the term “agents that decrease the amount and/or activity of BCOR” encompasses any natural or non-natural agent prepared, synthesized, manufactured, and/or purified by human that is capable of decreasing the expression level and/or activity of a BCOR polypeptide (and its fragments, domains, and/or motifs thereof, discussed herein). In some embodiments, the agent may decrease the binding/interaction between BCOR and its substrates or other binding partners. In other embodiments, the agent may decrease the expression of a BCOR polypeptide. In yet other embodiments, such agent may decrease BCOR's activity in enhancing the immune response against tumors. In still other embodiments, such inhibitors may increase the turnover rate, decrease the expression and/or the stability (e.g., the half-life), and/or change the cellular localization of BCOR, resulting in at least a decrease in BCOR levels and/or activity. Such agents may be any molecule, including but not limited to small molecule compounds, antibodies or intrabodies, RNA interfering (RNAi) agents (including at least siRNAs, shRNAs, microRNAs (miRNAs), piwi, and other well-known agents), and gene constructs that inhibit endogenous production of BCOR or its fragments inside cancer cells. Such agents may be specific to BCOR or also to at least one of the binding partners, including but not limited to BL6. Antibodies for detection of BCOR are commercially available (Cat. #AP33297PU-N, CF807724 (OriGene); ab135801, ab88112, ab129777, ab245423, among other, (Abcam); sc-514576 (Santa Cruz). RNA interference for BCOR polypeptides are well known and commercially available (e.g., human, rat, or mouse shRNA/siRNA products (Cat. #TL306415V, SR310311, TL504552, TL306415, TL306415V, TF306414, TL519839V, among others, and human or mouse gene knockout kit via CRISPR (Cat. #KN413468, KN502120) from Origene (Rockville, Md.), and siRNA/shRNA products (Cat. #sc-72635, sc-72636, sc-90861, sc-141680) from Santa Cruz Biotechonology (Dallas, Tex.). Methods for detection, purification, and/or inhibition of BCOR (e.g., by anti-BCOR antibodies) are also well known and commercially available (e.g., (Cat. #RC226424, RC213468L1V, RC226427, among others (OriGene). BCOR knockout human cell lines are also well known and available at Horizon (Cambridge, UK, Cat. #HZGHC004895c005, HZGHC004895c010).

The term “YAF2,” also known as “YY1-associated factor 2” refers to a zinc finger polypeptide or a YAF2-encoding polynucleotide that is involved in regulating transcription. YAF2 interacts with Yy1 and can promote its proteolysis. YAF2 also binds to MYC and inhibits MYC-mediated transactivation. Multiple alternatively spliced transcript variants are known. The term “YAF2” is intended to include fragments, variants (e.g., allelic variants) and derivatives thereof.

The nucleic acid and amino acid sequences of a representative human YAF2 is available to the public at the GenBank database (Gene ID 10138) and is shown in Table 1. Multiple transcript variants encoding several different isoforms have been found for YAF2, including at least four different human transcript variants generated by alternative splicing (see World Wide Web at uniprot.org/uniprot/Q8IY57). Human YAF2 variants include the transcript variant 3 encoding isoform 3 (NM_001190977.2 and NP_001177906.1), transcript variant 1 encoding isoform 1 (NM_001190979.2 and NP_001177908.1), transcript variant 4 encoding isoform 4 (NM_001190980.2 and NP_001177909.1), transcript variant 5 encoding isoform 5 (NM_005748.6 and NP_005739.2), transcript variant X1 encoding isoform (X1 XM_011537728.3 and XP_011536030.1), transcript variant X2 encoding isoform X2 (XM_024448792.1 and XP_024304560.1), transcript variant X3 encoding isoform X3 (XM_006719185.3 and XP_006719248.1), transcript variant X4 encoding isoform X4 (XM_011537729.2 and XP_011536031.1), and transcript variant X5 encoding transcript X5 (XM_017018670.2 and XP_016874159.1).

Nucleic acid and polypeptide sequences of YAF2 orthologs in organisms other than humans are well known and include, for example, chimpanzee (XM_001167723.5 and XP_001167723.1; XM_016923636.1 and XP_016779125.1; XM_016923633.1 and XP_016779122.1; XM_016923635.1 and XP_016779124.1; and XM_016923634.2 and XP_016779123.1), rhesus macaque (XM_015151457.2 and XP_015006943.1), and dog (XM_022410901.1 and XP_022266609.1; XM_014108587.2 and XP_013964062.1).

The term “YAF2 activity” includes the ability of a YAF2 polypeptide (and its fragments, domains, and/or motifs thereof, discussed herein) to bind its substrates, and/or mediate its transcription repression activity.

The term “YAF2 substrate(s)” refers to binding partners of a YAF2 polypeptide (and its fragments, domains, and/or motifs thereof, discussed herein), e.g., the proteins listed herein, including MYC and Yy1.

The term “YAF2 regulated pathway(s)” includes pathways in which YAF2 (and its fragments, domains, and/or motifs thereof, discussed herein) binds to at least one of its substrate, through which at least one cellular function and/or activity and/or cellular protein profiles is changed. YAF2-regulated pathways include at least those described herein, such as positive or negative regulation of histone modification.

The term “agents that decrease the copy number, the expression level, and/or the activity of YAF2,” or the term “agents that decrease the amount and/or activity of YAF2” encompasses any natural or non-natural agent prepared, synthesized, manufactured, and/or purified by human that is capable of decreasing the expression level and/or activity of a YAF2 polypeptide (and its fragments, domains, and/or motifs thereof, discussed herein). In some embodiments, the agent may decrease the binding/interaction between YAF2 and its substrates or other binding partners. In other embodiments, the agent may decrease the expression of a YAF2 polypeptide. In yet other embodiments, such agent may decrease YAF2 activity in enhancing the immune response against tumors. In still other embodiments, such inhibitors may increase the turnover rate, decrease the expression and/or the stability (e.g., the half-life), and/or change the cellular localization of YAF2, resulting in at least a decrease in YAF2 levels and/or activity. Such agents may be any molecule, including but not limited to small molecule compounds, antibodies or intrabodies, RNA interfering (RNAi) agents (including at least siRNAs, shRNAs, microRNAs (miRNAs), piwi, and other well-known agents), and gene constructs that inhibit endogenous production of YAF2 or its fragments inside cancer cells. Such agents may be specific to YAF2 or also to at least one of the binding partners, including but not limited to MYC and Yy1. Antibodies for detection of YAF2 are commercially available (Cat. #TA329295 TA329928 (OriGene); ab239150, ab177945, and ab250017 (Abcam); ABIN203352, ABIN1501785, ABIN5621096, ABIN6742302, ABIN6736123, ABIN2568985, ABIN2895187 (antibodies-online.com). RNA interference for YAF2 polypeptides are well known and commercially available (e.g., human, rat, or mouse shRNA/siRNA products (Cat. #TL316898V, SR306838, TR316898, TL503808V, TL708870V, TR708870, SR404295, TL708870, SR306838, TR503808, TL316898, TL503808, TL316898V) and human or mouse gene knockout kit via CRISPR (Cat. #KN414071, KN519535) from Origene (Rockville, Md.), and siRNA/shRNA products (Cat. #sc-95916, sc-155399) and human or mouse gene knockout kit via CRISPR (Cat. #sc-417274 among others) from Santa Cruz Biotechonology (Dallas, Tex.). Methods for detection, purification, and/or inhibition of YAF2 (e.g., by anti-YAF2 antibodies) are also well known and commercially available (e.g., (Cat. #TA331108 (OriGene); HG22835-ACGLN (Sino Biological US, Wayne, Pa.); 44489, C44489 (Signalway Antibody).

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “inhibit” includes the reduce, decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “interaction”, when referring to an interaction between two molecules, refers to the physical contact (e.g., binding) of the molecules with one another. Generally, such an interaction results in an activity (which produces a biological effect) of one or both of said molecules.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized.

The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM, IgG1, IgG2C, and the like) that is encoded by heavy chain constant region genes.

As used herein, the term “K_{D}” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

A “kit” is any manufacture (e.g. a package or container) comprising at least one reagent, e.g. a probe or small molecule, for specifically detecting and/or affecting the expression of a marker of the present invention. The kit may be promoted, distributed, or sold as a unit for performing the methods of the present invention. The kit may comprise one or more reagents necessary to express a composition useful in the methods of the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

The term “neoadjuvant therapy” refers to a treatment given before the primary treatment. Examples of neoadjuvant therapy can include chemotherapy, radiation therapy, and hormone therapy. For example, in treating breast cancer, neoadjuvant therapy can allows patients with large breast cancer to undergo breast-conserving surgery.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “pre-determined” biomarker amount and/or activity measurement(s) may be a biomarker amount and/or activity measurement(s) used to, by way of example only, evaluate a subject that may be selected for a particular treatment, evaluate a response to a treatment such as inhibitor(s) of the regulators of one or more biomarkers listed in Tables 1-5, in combination with an immunotherapy, and/or evaluate the disease state. A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., serum biomarker normalized to the expression of housekeeping or otherwise generally constant biomarker). The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The term “predictive” includes the use of a biomarker nucleic acid and/or protein status, e.g., over- or under-activity, emergence, expression, growth, remission, recurrence or resistance of tumors before, during or after therapy, for determining the likelihood of response of a cancer to inhibitor(s) of one or more biomarkers listed in Tables 1-5, in combination with an immunotherapy (e.g., treatment with a combination of such inhibitor and an immunotherapy, such as an immune checkpoint inhibitor). Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 110, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer (e.g., those responding to a particular inhibitor/immunotherapy combination therapy or those developing resistance thereto).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “probe” refers to any molecule which is capable of selectively binding to a specifically intended target molecule, for example, a nucleotide transcript or protein encoded by or corresponding to a biomarker nucleic acid. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules. The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., solid tumors, such as esophageal cancer and gastric cancer), development of one or more clinical factors, or recovery from the disease.

The term “response to immunotherapy” or “response to inhibitor(s) of one or more biomarkers listed in Tables 1-5, in combination with an immunotherapy” relates to any response of the hyperproliferative disorder (e.g., cancer) to an anti-cancer agent, such as an inhibitor of one or more biomarkers listed in Tables 1-5, and an immunotherapy, preferably to a change in tumor mass and/or volume after initiation of neoadjuvant or adjuvant therapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res. 1982; 42: 2159-2164), cell death assays (Weisenthal L M, Shoemaker R H, Marsden J A, Dill P L, Baker J A, Moran E M, Cancer Res. 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet (1994) 19: 82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 25% or more, for example, 30%, 40%, 50%, 60%, 70%, 80%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

RNA interfering agents, e.g., siRNA molecules, may be administered to a patient having or at risk for having cancer, to inhibit expression of a biomarker gene that is involved in downregulating MHC class I surface expression, such as HLA class I surface expression, in cancer and thereby treat, prevent, or inhibit cancer in the subject.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a nonspecific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

As used herein, the term “protein complex” means a composite unit that is a combination of two or more proteins formed by interaction between the proteins. Typically, but not necessarily, a “protein complex” is formed by the binding of two or more proteins together through specific non-covalent binding interactions. However, covalent bonds may also be present between the interacting partners. For instance, the two interacting partners can be covalently crosslinked so that the protein complex becomes more stable. The protein complex may or may not include and/or be associated with other molecules such as nucleic acid, such as RNA or DNA, or lipids or further cofactors or moieties selected from a metal ions, hormones, second messengers, phosphate, sugars. A “protein complex” encompassed by the present invention may also be part of or a unit of a larger physiological protein assembly.

The term “isolated protein complex” means a protein complex present in a composition or environment that is different from that found in nature, in its native or original cellular or body environment. Preferably, an “isolated protein complex” is separated from at least 50%, more preferably at least 75%, most preferably at least 90% of other naturally co-existing cellular or tissue components. Thus, an “isolated protein complex” may also be a naturally existing protein complex in an artificial preparation or a non-native host cell. An “isolated protein complex” may also be a “purified protein complex”, that is, a substantially purified form in a substantially homogenous preparation substantially free of other cellular components, other polypeptides, viral materials, or culture medium, or, when the protein components in the protein complex are chemically synthesized, free of chemical precursors or by-products associated with the chemical synthesis. A “purified protein complex” typically means a preparation containing preferably at least 75%, more preferably at least 85%, and most preferably at least 95% of a particular protein complex. A “purified protein complex” may be obtained from natural or recombinant host cells or other body samples by standard purification techniques, or by chemical synthesis.

The term “modified polypeptide” or “modified protein complex” refers to a polypeptide or a protein complex present in a composition that is different from that found in nature in its native or original cellular or body environment. The term “modification” as used herein refers to all modifications of a protein or protein complex encompassed by the present invention including cleavage and addition or removal of a group. In some embodiments, the “modified polypeptide” or “modified protein complex” comprises at least one modification (e.g., fragment, mutation, and the like) or subunit that is modified, i.e., different from that found in nature, in its native or original cellular or body environment. The “modified subunit” may be, e.g., a derivative or fragment of the native subunit from which it derives.

The term “activity” when used in connection with proteins or protein complexes means any physiological or biochemical activities displayed by or associated with a particular protein or protein complex including but not limited to activities exhibited in biological processes and cellular functions, ability to interact with or bind another molecule or a moiety thereof, binding affinity or specificity to certain molecules, in vitro or in vivo stability (e.g., protein degradation rate, or in the case of protein complexes ability to maintain the form of protein complex), antigenicity and immunogenicity, enzymatic activities, etc. Such activities may be detected or assayed by any of a variety of suitable methods as will be apparent to skilled artisans.

As used herein, the term “interaction antagonist” means a compound that interferes with, blocks, disrupts or destabilizes a protein-protein interaction; blocks or interferes with the formation of a protein complex, or destabilizes, disrupts or dissociates an existing protein complex.

The term “interaction agonist” as used herein means a compound that triggers, initiates, propagates, nucleates, or otherwise enhances the formation of a protein interaction; triggers, initiates, propagates, nucleates, or otherwise enhances the formation of a protein complex; or stabilizes an existing protein complex.

The term “PRC1.1 complex,” or “polycomb repressive complex 1.1” refers to a complex of proteins comprising USP7, KDM2B, BCOR or BCORL1, RING1A, RING1B, RYBP/YAF2, PCGF1, and SKP1. Loss of PRC1.1 has been shown to accelerate development of sonic hedgehog-driven medulloblastoma (Kutscher et al. (2020) bioRxiv 2020.02.06.938035). The complex has been studied in the context of the commitment of hematopoietic stem and progenitor cells (HSPCs). PRC1.1 insufficiency in these cells induced myeloid-based differentiation, leading to the myeloid malignancies (Iwama (2018) Exp. Hematol. 64:S39).

The term “subject” refers to any healthy animal, mammal or human, or any animal, mammal or human afflicted with a cancer, e.g., brain, lung, ovarian, pancreatic, liver, breast, prostate, and/or colorectal cancers, melanoma, multiple myeloma, and the like. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “synergistic effect” refers to the combined effect of two or more anti-cancer agents (e.g., inhibitor(s) of one or more biomarkers listed in Tables 1-5, in combination with an immunotherapy) can be greater than the sum of the separate effects of the anti-cancer agents/therapies alone.

The term “T cell” includes CD4⁺ T cells and CD8⁺ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

In some embodiments, the terms “therapeutically effective amount” and “effective amount” may be that amount of a compound, material, or composition comprising an agent that is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%0, 60%, 70%, 80%, 90%, 100%, 200%, 300%, 400%, 500%, 600%, 700%, 800%, 900%, 1000% or more increased for the agent relative to no administration of the agent. Also, similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g. splicing), if any, of the RNA transcript, and reverse transcription of the RNA transcript.

As used herein, the term “unresponsiveness” includes refractivity of cancer cells to therapy or refractivity of therapeutic cells, such as immune cells, to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134).

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the loci and biomarkers of the present invention (e.g., biomarkers listed in Tables 1 and 2) are well-known in the art and readily available on publicly available databases, such as the National Center for Information (NCBI). For example, exemplary nucleic acid and amino acid sequences derived from publicly available sequence databases are provided below and include, for example, PCT Publ. WO 2014/022759, which is incorporated herein in its entirety by this reference.

TABLE 5
USP7
BCORL1
PCGF1
KDM2B
SKP1
RING1A
RING1B
RYBP
YAF2
BCOR
MYCL
SEQ ID NO: 1 Human USP7 Transcript Variant 1 cDNA seauence (NM_003470.3, CDS
region from position 622-3930)

| | |
|---|---|
| 1 | gacatttcac gccgccgcca ttttgagagc gagccgagcc gagctgccgg gcgccgcgtc |
| 61 | cgctgccgga gccccgacga cgacgccgag gaggcggagg ccgcggctct cggaacgcgg |
| 121 | ccggcccgtc gcccgcccgc tccgccgctc ccgccggccc cagggcccgc aggcccgccg |
| 181 | cggccggcca ggcctcccgt ccgccgcgcc cggcccgagg cggggctgac tccgcggccc |
| 241 | ccgaccggcc gccctccgcc cccggccggc ccgcggcccc gcagccccgg ccccggcccc |
| 301 | ggcggcggga ggcgggcccc gcggcggcgg cggcggcggc cgcagcgagc gacgaggccg |
| 361 | cccggcccgg cccgccggcc gcccgcccgc ctcggcgccg agattgggcg aatcgcgagc |
| 421 | aagtacgtgc gcgtctccct gccgccgccg ccgcccgccg cgggccgccc cggggccgcc |
| 481 | gtcgccgacg acgcgcggga ggaggaggag gaggccgccc cgccgccgcc gccgccgccg |
| 541 | ccgccccggc tcgccgccgc ccgcccgccg ggctcgcagc cccggccccc ggccgcaggc |
| 601 | gaggcccagg ccgcggccga catgaaccac cagcagcagc agcagcagca gaaagcgggc |
| 661 | gagcagcagt tgagcgagcc cgaggacatg gagatggaag cgggagatac agatgaccca |
| 721 | ccaagaatta ctcagaaccc tgtgatcaat gggaatgtgg ccctgagtga tggacacaac |
| 781 | accgcggagg aggacatgga ggatgacacc agttggcgct ccgaggcaac ctttcagttc |
| 841 | actgtggagc gcttcagcag actgagtgag tcggtcctta gccctccgtg ttttgtgcga |
| 901 | aatctgccat ggaagattat ggtgatgcca cgcttttatc cagacagacc acaccaaaaa |
| 961 | agcgtaggat tctttctcca gtgcaatgct gaatctgatt ccacgtcatg gtcttgccat |
| 1021 | gcacaagcag tgctgaagat aataaattac agagatgatg aaaagtcgtt cagtcgtcgt |
| 1081 | attagtcatt tgttcttcca taaagaaaat gattggggat tttccaattt tatggcctgg |
| 1141 | agtgaagtga ccgatcctga gaaaggattt atagatgatg acaaagttac ctttgaagtc |
| 1201 | tttgtacagg cggatgctcc ccatggagtt gcgtgggatt caaagaagca cacaggctac |
| 1261 | gtcggcttaa agaatcaggg agcgacttgt tacatgaaca gcctgctaca gacgttattt |
| 1321 | ttcacgaatc agctacgaaa ggctgtgtac atgatgccaa ccgaggggga tgattcgtct |
| 1381 | aaaagcgtcc ctttagcatt acaaagagtg ttctatgaat tacagcatag tgataaacct |
| 1441 | gtaggaacaa aaaagttaac aaagtcattt gggtgggaaa ctttagatag cttcatgcaa |
| 1501 | catgatgttc aggagctttg tcgagtgttg ctcgataatg tggaaaataa gatgaaaggc |
| 1561 | acctgtgtag agggcaccat acccaaatta ttccgcggca aaatggtgtc ctatatccag |
| 1621 | tgtaaagaag tagactatcg gtctgataga agagaagatt attatgatat ccagctaagt |
| 1681 | atcaaaggaa agaaaaatat atttgaatca tttgtggatt atgtggcagt agaacagctc |
| 1741 | gatggggaca ataaatacga cgctggggaa catggcttac aggaagcaga gaaaggtgtg |
| 1801 | aaattcctaa cattgccacc agtgttacat ctacaactga tgagatttat gtatgaccct |
| 1861 | cagacggacc aaaatatcaa gatcaatgat aggtttgaat tcccagagca gttaccactt |
| 1921 | gatgaatttt tgcaaaaaac agatcctaag gaccctgcaa attatattct tcatgcagtc |
| 1981 | ctggttcata gtggagataa tcatggtgga cattatgtgg tttatctaaa ccccaaaggg |
| 2041 | gatggcaaat ggtgtaaatt tgatgacgac gtggtgtcaa ggtgtactaa agaggaagca |
| 2101 | attgagcaca attatggggg tcacgatgac gacctgtctg ttcgacactg cactaatgct |
| 2161 | tacatgttag tctacatcag ggaatcaaaa ctgagtgaag ttttacaggc ggtcaccgac |
| 2221 | catgatattc ctcagcagtt ggtggagcga ttacaagaag agaaaaggat cgaggctcag |
| 2281 | aagcggaagg agcggcagga agcccatctc tatatgcaag tgcagatagt cgcagaggac |
| 2341 | cagttttgtg gccaccaagg gaatgacatg tacgatgaag aaaaagtgaa atacactgtg |
| 2401 | ttcaaagtat tgaagaactc ctcgcttgct gagtttgttc agagcctctc tcagaccatg |
| 2461 | ggatttccac aagatcaaat tcgattgtgg cccatgcaag caaggagtaa tggaacaaaa |
| 2521 | cgaccagcaa tgttagataa tgaagccgac ggcaataaaa caatgattga gctcagtgat |
| 2581 | aatgaaaacc cttggacaat attcctggaa acagttgatc ccgagctggc tgctagtgga |
| 2641 | gcgaccttac ccaagtttga taaagatcat gatgtaatgt tatttttgaa gatgtatgat |
| 2701 | cccaaaacgc ggagcttgaa ttactgtggg catatctaca caccaatatc ctgtaaaata |
| 2761 | cgtgacttgc tcccagttat gtgtgacaga gcaggattta ttcaagatac tagccttatc |
| 2821 | ctctatgagg aagttaaacc gaatttaaca gagagaattc aggactatga cgtgtctctt |
| 2881 | gataaagccc ttgatgaact aatggatggt gacatcatag tatttcagaa ggatgaccct |
| 2941 | gaaaatgata acagtgaatt acccaccgca aaggagtatt tccgagatct ctaccaccgc |
| 3001 | gttgatgtca ttttctgtga taaaacaatc cctaatgatc ctggatttgt ggttacgtta |
| 3061 | tcaaatagaa tgaattattt tcaggttgca aagacagttg cacagaggct caacacagat |
| 3121 | ccaatgttgc tgcagttttt caagtctcaa ggttataggg atggcccagg taatcctctt |
| 3181 | agacataatt atgaaggtac tttaagagat cttctacagt tcttcaagcc tagacaacct |
| 3241 | aagaaacttt actatcagca gcttaagatg aaaatcacag actttgagaa caggcgaagt |
| 3301 | tttaaatgta tatggttaaa cagccaattt agggaagagg aaataacact atatccagac |
| 3361 | aagcatgggt gtgtccggga cctgttagaa gaatgtaaaa aggccgtgga gcttggggag |
| 3421 | aaagcatcag ggaaacttag gctgctagaa attgtaagct acaaaatcat tggtgttcat |
| 3481 | caagaagatg aactattaga atgtttatct cctgcaacga gccggacgtt tcgaatagag |
| 3541 | gaaatccctt tggaccaggt ggacatagac aaagagaatg agatgcttgt cacagtggcg |
| 3601 | catttccaca aagaggtctt cggaacgttc ggaatcccgt ttttgctgag gatacaccag |
| 3661 | ggcgagcatt ttcgagaagt gatgaagcga atccagagcc tgctggacat ccaggagaag |
| 3721 | gagtttgaga agtttaaatt tgcaattgta atgatgggcc gacaccagta cataaatgaa |
| 3781 | gacgagtatg aagtaaattt gaaagacttt gagccacagc ccggtaatat gtctcatcct |
| 3841 | cggccttggc tagggctcga ccacttcaac aaagccccaa agaggagtcg ctacacttac |
| 3901 | cttgaaaagg ccattaaaat ccataactga tttccaagct ggtgtgttca aggcgaggac |
| 3961 | ggtgtgtggg tggcccctta acagcctaga actttggtgc acgtgccctc tagccgaagt |
| 4021 | cttcagcaag aggattcgct gctggtgtta attttatttt attgaggctg ttcagtttgg |
| 4081 | cttctctgta tctattgact gccctttttg agcaaaatga agatgttttt ataaagcttg |
| 4141 | gatgccaatg agagttattt tatggtaacc acagtgcaag gcaactgtca gcgcaatggg |
| 4201 | tggagaagag gtagtggatc gggggtccct ggctcaaggt ctctgggctg tccctagtgg |
| 4261 | gcacgagtgg ctcggctgcc ttcctggggt cccgtgcacc agccctgcag ctagcaagtc |
| 4321 | ttgtgtttag gctcgtctga cctatttcct tcagttatac tttcaatgac cttttgtgca |
| 4381 | tctgttaagg caaaacagag aaactcacaa cctaataaat agcgctcttc ccttcattgt |
| 4441 | gtgcattgtc ggcccttcct cgggttctcc tcctccagct gcctgggggc tttttaataa |
| 4501 | acttgtctca cctcgtcagc cactactgtc tgcagcccct ttgcaaagtg gatgcactga |
| 4561 | atacagtccg gacagacatt gtgggggtct ttttattaaa tcaagaacat tgttaaattc |
| 4621 | aattaaggtt tactctgctg ccttggcaga cttacgatct caacagttca tacgagcagg |
| 4681 | tgaaaggatt ataaatagaa tttcgttaaa gtggaacaga cgacaagaaa gccttttagc |
| 4741 | gaagagggca tctcactagt ggttagtaag ctgtcgactt tgtaaaaaag ttaaaaatga |
| 4801 | aaaaaaaagg aaaaatgaat tgtatattta atgaatgaac atgtacaatt tgccactggg |
| 4861 | aggaggttcc tttttgttgg gtgagtctgc aagtgaattt cactgatgtt gatattcatt |
| 4921 | gtgtgtagtt ttatttcggt cccagccccg tttcctttta ttttggagct aatgccagct |
| 4981 | gcgtgtctag ttttgagtgc agtaaaatag aatcagcaaa tcactcttat ttttcatcct |
| 5041 | tttccggtat tttttgggtt gtttctgtgg gagcagtgta caccaactct tcctgtatat |
| 5101 | tgcctttttg ctggaaaatg ttgtatgttg aataaaattt tctataaaaa ttataattca |
| 5161 | gtgagttacg tggaagtgga ggaagatttc tactctccct ggaaacaggc ctgggaaacc |
| 5221 | ttggcatttg taacaaggtt tcactgagat gtacttttcc ttctaattcc gttttgcggg |
| 5281 | ggcagggtct cttgtttctt tttttttttt tttttttttt tagcctctaa ctagtcacat |
| 5341 | ttactcttaa gaaatgaaag gttttccagg agagaactgt gtacaaataa ggtgactgga |
| 5401 | gatgtgacct gatgtgtcac gaggcccttc ggggcggcag gcgctatcgt gggcgtggtc |
| 5461 | cttgcaccgt cccatcggcc ttgccttcca gctccgtggc acggtttcct ggtctttggg |
| 5521 | ccagtgtgta ccttggagtg acttcctttc tcaacttcca ctgcagtgtg tgtgccttct |
| 5581 | gctctgagag ctgccttgtg acccgtgtga tagaaagcag ggagtgaggg tccccgcgga |
| 5641 | cctggccctt ccctccttcc tcccccagaa agaggagtta gagcaggggt gcgagagccg |
| 5701 | ttcgctgtgg gtttgtcttt gaacaaacat taaggtgtct tgtttttgtt ctgggctggg |
| 5761 | ggttggctgt agtcttaggt aactgaaagt tcctactctc ccttaaggta ttaaatgact |
| 5821 | ctttttccaa a |

SEQ ID NO: 2 Human Ubiquitin Carboxyl-terminal Hydrolase 7 Isoform 1 (Encoded by
Transcript Variant 1) Amino Acid Sequence (NP_003461.2)

| | |
|---|---|
| 1 | mnhqqqqqqq kageqqlsep edmemeagdt ddppritqnp vingnvalsd ghntaeedme |
| 61 | ddtswrseat fqftverfsr isesvlsppc fvrnlpwkim vmprfypdrp hqksvgffiq |
| 121 | cnaesdstsw schaqavlki inyrddeksf srrishlffh kendwgfsnf mawsevtdpe |
| 181 | kgfidddkvt fevfvqadap hgvawdskkh tgyvglknqg atcymnsllq tifftnqlrk |
| 241 | avymmptegd dssksvplal qrvfyelqhs dkpvgtkklt ksfgwetlds fmqhdvqelc |
| 301 | rvlldnvenk mkgtcvegti pklfrgkmvs yiqckevdyr sdrredyydi qlsikgkkni |
| 361 | fesfvdyvav eqldgdnkyd agehglqeae kgvkfitlpp vlhlqlmrfm ydpqtdqnik |
| 421 | indrfefpeq ipldefiqkt dpkdpanyil havivhsgdn hgghyvvyln pkgdgkwckf |
| 481 | dddvvsrctk eeaiehnygg hdddlsvrhc tnaymlvyir esklsevlqa vtdhdipqql |
| 541 | verlqeekri eaqkrkerqe ahlymqvqiv aedqfcghqg ndmydeekvk ytvfkvikns |
| 601 | slaefvqsls qtmgfpqdqi rlwpmqarsn gtkrpamldn eadgnktmie isdnenpwti |
| 661 | fletvdpela asgatipkfd kdhdvmlflk mydpktrsin ycghiytpis ckirdllpvm |
| 721 | cdragfiqdt slilyeevkp nlteriqdyd vsidkaldel mdgdiivfqk ddpendnsel |
| 781 | ptakeyfrdl yhrvdvifcd ktipndpgfv vtlsnrmnyf qvaktvaqrl ntdpmllqff |
| 841 | ksqgyrdgpg nplrhnyegt irdllqffkp rqpkklyyqq ikmkitdfen rrsfkciwln |
| 901 | sqfreeeitl ypdkhgcvrd lleeckkave Igekasgklr lleivsykii gvhqedelle |
| 961 | clspatsrtf rieeipldqv didkenemlv tvahfhkevf gtfgipfllr ihqgehfrev |
| 1021 | mkriqslldi qekefekfkf aivmmgrhqy inedeyevnl kdfepqpgnm shprpwlgld |
| 1081 | hfnkapkrsr ytylekaiki hn |

SEQ ID NO: 3 Human KDM2B Transcript Variant 1 cDNA Sequence
(NM_032590.5, CDS region from position 113-4123)

| | |
|---|---|
| 1 | gtacgtgtgt gtgtccacat ctttgagtgc cgggagttta aaagttaggc agtccttata |
| 61 | ggtatggaag ccgagctaat ttccttctga gccccccaaa tgcctcctcc acatggcggg |
| 121 | tccgcaaatg gggggatctg cagaggatca ccccccacga aaaagacatg cagcagaaaa |
| 181 | gcaaaaaaag aaaacagtta tatatacaaa atgctttgaa tttgagtcgg ccacacagcg |
| 241 | cccgattgac cgccagcgat acgacgagaa cgaggacttg tcggacgtgg aggagatcgt |
| 301 | cagcgtccgc ggcttcagcc tggaggagaa gcttcgcagc cagctgtacc agggggactt |
| 361 | cgtgcacgcc atggagggca aagatttcaa ctatgagtac gtacagagag aagctctcag |
| 421 | ggttcccctg atatttcgag aaaaggatgg actgggaatt aagatgcctg accctgattt |
| 481 | cacagtccga gacgtcaaac tcctagtggg gagccggcgg cttgtggacg tgatggatgt |
| 541 | gaacacccag aagggcacgg agatgagcat gtcccagttt gtgcgttact acgagacgcc |
| 601 | cgaggcccag cgggacaagc tgtacaacgt catcagccta gagttcagcc acaccaagct |
| 661 | ggagcacttg gtcaagcgtc cgactgtggt agacctggtg gactgggtgg acaacatgtg |
| 721 | gccccagcat ctgaaggaga agcagacaga agccacgaac gccattgcag agatgaagta |
| 781 | cccgaaagtg aaaaagtact gtctgatgag cgtgaaaggt tgtttcaccg acttccacat |
| 841 | cgactttgga ggcacttccg tttggtacca tgttttccgg ggtgggaaga ttttttggct |
| 901 | gattcctcca acgctgcaca atttggcgct gtacgaggag tgggtgctgt caggcaaaca |
| 961 | gagtgacatc tttctgggag accgtgtgga acgatgccaa agaattgagc tgaagcaggg |
| 1021 | ctacacattt ttcatccctt ccggttggat ccatgccgtc tacacccctg tagactcttt |
| 1081 | ggtgttcggc ggaaacatcc tgcacagctt taacgtgccc atgcagctgc ggatctacga |
| 1141 | gatcgaggac aggacgcggg tgcagcccaa attccgttac cccttctact atgagatgtg |
| 1201 | ctggtatgtc ctggagagat acgtgtactg tgtgacccag cgctcccacc tcactcagga |
| 1261 | ataccagagg gagtcgatgc ttattgatgc cccgaggaag cccagcatag acggcttctc |
| 1321 | ttcggattcc tggctggaga tggaggagga ggcctgtgat cagcagcctc aggaggagga |
| 1381 | ggagaaggac gaggagggcg agggcaggga cagggcaccc aaaccgccca ccgatggctc |
| 1441 | cacttcaccc accagcacgc cctctgagga ccaggaggcc ctcgggaaga agcccaaagc |
| 1501 | acctgccctg cgattcctca aaaggacttt gtctaatgag tcggaggaaa gtgtgaagtc |
| 1561 | caccacattg gccgtagact accccaagac ccccaccggc tctcccgcca cggaggtctc |
| 1621 | tgccaaatgg acccatctca ctgagtttga actgaagggc ctgaaagctc tggtggagaa |
| 1681 | actggaatcc ctcccggaga acaagaagtg tgtccccgag ggcatcgagg acccccaggc |
| 1741 | actcctggag ggtgtgaaga acgtcctgaa ggagcacgca gatgatgacc ctagtctggc |
| 1801 | catcactggg gtccctgtgg tgacttggcc aaagaagact ccaaagaacc gggctgtggg |
| 1861 | tcggcccaag gggaagctgg gcccggcctc cgcggtgaag ttggccgcca accggacaac |
| 1921 | ggcaggagct cggcggcgcc ggacgcgatg ccgcaagtgc gaggcctgcc tgcggaccga |
| 1981 | gtgcggagag tgccacttct gcaaggacat gaagaagttc gggggccccg ggcgcatgaa |
| 2041 | gcagagctgc atcatgcggc agtgcatcgc gccagtgctg ccccacaccg ccgtgtgcct |
| 2101 | tgtgtgtggc gaggcgggga aggaagacac ggtggaagag gaggaaggca agtttaacct |
| 2161 | catgctcatg gagtgctcca tctgcaatga aatcatccac cctggatgcc ttaagattaa |
| 2221 | ggagtcagag ggtgtggtca acgacgagct tccaaactgc tgggagtgtc cgaagtgtaa |
| 2281 | ccacgccggc aagaccggga aacaaaagcg tggccctggc tttaagtacg cctccaacct |
| 2341 | gcccggctcc ctgctcaagg agcagaagat gaaccgggac aacaaggaag ggcaggaacc |
| 2401 | tgccaagcgg aggagtgagt gtgaggaggc gccccggcgc aggtcggatg agcactcgaa |
| 2461 | gaaggtgccg ccggacggcc ttctgcgcag aaagtctgac gacgtgcacc tgaggaagaa |
| 2521 | gcggaaatac gagaagcccc aggagctgag tggacgcaag cgggcctcat cgcttcaaac |
| 2581 | gtcccccggt tcctcctctc acctctcgcc gaggccccct ctaggcagca gcctcagccc |
| 2641 | ctggtggaga tccagtctca cttacttcca gcagcagctc aaacctggca aagaagataa |
| 2701 | gcttttcagg aaaaagcggc ggtcctggaa gaacgccgag gaccgcatgg cgctggccaa |
| 2761 | caagcccctc cggcgcttca agcaggaacc cgaggacgaa ctgcccgagg cgccccccaa |
| 2821 | gaccagggag agcgaccact cccgctccag ctcccccacc gcgggaccca gcaccgaagg |
| 2881 | ggccgagggc ccggaggaga agaagaaggt gaagatgcgc cggaagcggc ggcttcccaa |
| 2941 | caaggagctg agcagggagc tgagcaagga gctcaaccac gagatccaga ggacggagaa |
| 3001 | cagcctggcc aacgagaacc agcagcccat caagtcggag cctgagagcg agggcgagga |
| 3061 | gcccaagcgg cccccgggca tctgcgagcg tccccaccgc ttcagcaagg ggctcaacgg |
| 3121 | caccccccgg gagctgcggc accagctggg gcccagcctg cgcagcccgc cccgtgtcat |
| 3181 | ctcccggccc ccaccctccg tgtccccgcc caagtgtatc cagatggagc gccatgtgat |
| 3241 | ccggccaccc cccatcagcc ccccgcctga ctcgctaccc ctggacgatg gggcagccca |
| 3301 | cgtcatgcac agggaggtgt ggatggccgt cttcagctac ctcagccacc aagacctgtg |
| 3361 | tgtgtgcatg cgggtctgca ggacctggaa ccgctggtgc tgcgataagc ggttgtggac |
| 3421 | ccgcattgac ctgaaccact gcaagtctat cacacccctg atgctgagtg gcatcatccg |
| 3481 | gcgacagccc gtctccctcg acctcagctg gaccaatatc tccaagaagc agctgagctg |
| 3541 | gctcatcaac cggctgcctg ggctccggga cttggtgctg tcaggctgct catggatcgc |
| 3601 | ggtctcggcc ctttgcagct ccagttgtcc gctgctccgg accctggatg tccagtgggt |
| 3661 | ggagggacta aaggatgccc agatgcggga tctcctgtcc ccgcccacag acaacaggcc |
| 3721 | aggtcagatg gacaatcgga gcaagctccg gaacatcgtg gagctgcgcc tggcaggcct |
| 3781 | ggacatcaca gatgcctccc tgcggctcat catccgccac atgcccctgc tctccaagct |
| 3841 | ccacctcagt tactgtaacc acgtcaccga ccagtctatc aacctgctca ctgctgttgg |
| 3901 | caccaccacc cgagactcct taaccgagat caacctgtct gactgcaata aggtcactga |
| 3961 | tcagtgcctg tccttcttca aacgctgtgg aaacatctgt catattgacc tgaggtactg |
| 4021 | caagcaagtc accaaggaag gctgtgagca gttcatagcc gagatgtctg tgagtgtcca |
| 4081 | gtttgggcaa gtagaagaaa aactcctgca aaaactgagt tagtccaagg ataagtatgt |
| 4141 | aaatacgggg cgggctctgg gaggggagag actttacaaa aatgagggct tttattttcc |
| 4201 | atttggaacg tgggacaaca gaccacaacg caattccatt ttgcaagtct ttccaaggga |
| 4261 | gaagctgttc aaccacccgt ttgggggatg agtgagccga cactttcctt tggtctttct |
| 4321 | gaatcgtaac tgcactgctt tctggaccat ttctaaggcg gcctttacaa gaagacattc |
| 4381 | ctgtcggaga ggagggtgga cttcggagaa attctcatac tgaagcatga gcttaggagt |
| 4441 | ttctgttagt ggtagtggtg ttttggacac ttcattcctt gcaacaccga ggttttgggt |
| 4501 | gttgacataa agtggaccac acaccacatc tgctgccgtc ttgacacttt tttttgtttg |
| 4561 | gttggttttg ttacatctta cattatgcag aactattttt gtacaaattg tttaaaagtt |
| 4621 | atttatgcaa ggtttgaatg cataccagtg tttttattgt tttgagattg ccaattttcc |
| 4681 | tgatttcctt aaggtaggag agaatttaac gtgtacttca tcgacacaac ccatctacaa |
| 4741 | atgtgcccag atctaacaaa gtaggctaag accttccact taaaagcatg tttaactgga |
| 4801 | agttgagagt ctgctttgta cctcaagagt tacatgagca tgttgtggat aaatgtaaat |
| 4861 | tatagtcaaa gtaagatact ctgccaagtt tcctctgtag agaattcact tttctcaaat |
| 4921 | tttaaaattt cgacttcagc ctttgcactc aggaggttct gctccagcat gagctcttgt |
| 4981 | acttacatag atctaattta tacagtgagt caagacgtag aataaatgct cccacatagc |
| 5041 | ctttcttttg cttttgcttc tctcctctga agtgtgagtt gagttctcat ttaggtttgt |
| 5101 | aacatggcta tttcctagtt gtaaagttct gcatttataa gtgccattgt tgtaaggtgg |
| 5161 | tgtttcctag accttccctg atgcgatttt acctttgttg aatttgtata aacaattgta |
| 5221 | caaaaaaaac cactcttgaa ctttgagggt ttctgttcta ggagtggact agaagtttaa |
| 5281 | gcccagagtc agtaaacact gttttgaagt ccaaa |

SEQ ID NO: 4 Human KDM2B (Encoded by Transcript Isoform A) Amino Acid
Sequence (NP_115979.3)

| | |
|---|---|
| 1 | magpqmggsa edhpprkrha aekqkkktvi ytkcfefesa tqrpidrqry denedlsdve |
| 61 | eivsvrgfsl eeklrsqlyq gdfvhamegk dfnyeyvqre alrvplifre kdglgikmpd |
| 121 | pdftvrdvkl lvgsrrlvdv mdvntqkgte msmsqfvryy etpeaqrdkl ynvislefsh |
| 181 | tklehivkrp tvvdlvdwvd nmwpqhlkek qteatnaiae mkypkvkkyc lmsvkgcftd |
| 241 | fhidfggtsv wyhvfrggki fwlipptlhn lalyeewvls gkqsdiflgd rvercqriel |
| 301 | kqgytffips gwihavytpv dsivfggnil hsfnvpmqlr iyeiedrtrv qpkfrypfyy |
| 361 | emcwyvlery vycvtqrshl tqeyqresml idaprkpsid gfssdswiem eeeacdqqpq |
| 421 | eeeekdeege grdrapkppt dgstsptstp sedqealgkk pkapalrflk rtlsnesees |
| 481 | vksttlavdy pktptgspat evsakwthlt efelkglkal vekleslpen kkcvpegied |
| 541 | pqallegvkn vlkehadddp slaitgvpvv twpkktpknr avgrpkgklg pasavklaan |
| 601 | rttagarrrr trcrkceacl rtecgechfc kdmkkfggpg rmkqscimrq ciapvlphta |
| 661 | vclvcgeagk edtveeeegk fnlmlmecsi cneiihpgcl kikesegvvn delpncwecp |
| 721 | kcnhagktgk qkrgpgfkya snlpgsllke qkmnrdnkeg qepakrrsec eeaprrrsde |
| 781 | hskkvppdgl lrrksddvhl rkkrkyekpq elsgrkrass lqtspgsssh isprpplgss |
| 841 | lspwwrsslt yfqqqlkpgk edklfrkkrr swknaedrma lankplrrfk qepedelpea |
| 901 | ppktresdhs rsssptagps tegaegpeek kkvkmrrkrr lpnkelsrel skelnheiqr |
| 961 | tenslanenq qpiksepese geepkrppgi cerphrfskg lngtprelrh qlgpslrspp |
| 1021 | rvisrpppsv sppkciqmer hvirpppisp ppdslplddg aahvmhrevw mavfsylshq |
| 1081 | dlcvcmrvcr twnrwccdkr iwtridinhc ksitpimlsg iirrqpvsld lswtniskkq |
| 1141 | lswlinrlpg irdlvlsgcs wiavsalcss scpllrtldv qwveglkdaq mrdllspptd |
| 1201 | nrpgqmdnrs klrnivelrl aglditdasl rliirhmpll sklhlsycnh vtdqsinllt |
| 1261 | avgtttrdsl teinlsdcnk vtdqclsffk rcgnichidl ryckqvtkeg ceqfiaemsv |
| 1321 | svqfgqveek llqkls |

SEQ ID NO: 5 Human BCORL1 Transcript Variant 1 (NM_021946.5, CDS region from
position 173-5308)

| | |
|---|---|
| 1 | actctttcgc tcgccgcggc tgctgccagt gtgtggctct gtctctcctc cgctttgctg |
| 61 | agccctccct tcttcctctc agttcctaga gtccgaccgc cgccgccgcc gagagagagg |
| 121 | agaaggaggg ggagtggcca cagcaggtcc tatctggtgg tgagtggctg tcatgatctc |
| 181 | tacagcaccg ctctacagcg gcgtgcacaa ctggaccagt tctgaccgga ttcgcatgtg |
| 241 | tggcatcaac gaggagagaa gagcacctct ttctgatgag gagtcaacga caggcgactg |
| 301 | ccagcacttt ggatctcagg agttttgtgt cagcagcagt ttttccaagg tggagctcac |
| 361 | ggcagttgga agtggcagca atgcccgggg ggcagaccca gatggcagtg ctacagaaaa |
| 421 | acttgggcac aagtcagaag acaagcctga cgatccccag ccaaaaatgg actacgctgg |
| 481 | gaacgtggca gaggctgagg gcctcttggt gcccctgagc agcccaggag acgggctcaa |
| 541 | gcttcccgca tctgacagcg ccgaggccag caacagcagg gccgactgct cctggactcc |
| 601 | actcaacacc caaatgagca aacaggttga ctgctcaccc gccggagtaa aggctttgga |
| 661 | ctctcggcaa ggtgttggag agaagaatac tttcattttg gcaactctgg gaactggagt |
| 721 | ccctgtggag gggaccctgc ccctggttac cactaacttc agtcctctgc cagcccctat |
| 781 | ctgtccccct gctcccggtt cggcctctgt gccccactct gttccagatg cattccaggt |
| 841 | tcccctctcc gtccctgccc cagtccccca ttcagggctt gttccagtcc aagttgccac |
| 901 | ttcggttcca gctccttccc ctcccttagc acctgtcccg gctctggctc cagcgccacc |
| 961 | gtcagtgccc acgctcatct ctgactcgaa ccccctttct gtttcggcct cagtcttggt |
| 1021 | gcctgtgcca gcttctgctc ccccttcagg cccggttccc ttgtcggctc cagctcctgc |
| 1081 | cccgctttca gtcccagttt cagctcctcc cttggctctc atccaggctc ctgtgccccc |
| 1141 | ttcagctccg accttggttc tcgctcccgt ccccactccg gttctggctc ccatgccagc |
| 1201 | atccacgcct ccagcggccc ctgcccctcc gtctgtgccc atgcccactc caaccccatc |
| 1261 | ttccggccca ccttctaccc ccaccctcat ccccgccttt gctcctacac cggtgcctgc |
| 1321 | acccacccca gcccccatct ttactccagc ccctacaccc atgcctgctg ccacgccagc |
| 1381 | tgccattccc acctctgcac ccatcccggc ctccttcagt ttgagtagag tgtgctttcc |
| 1441 | tgcagctcag gcaccagcta tgcaaaaagt ccccctgtcc tttcagccag ggacagtgct |
| 1501 | gaccccgagc cagccgctgg tatatatccc gcctccaagc tgtgggcagc cactcagtgt |
| 1561 | ggccacactg ccaaccactc taggggtttc ctccactctt acgctccctg tcctgccgtc |
| 1621 | ctacctgcag gacaggtgtc tcccaggcgt gctagcctcc cccgagctcc gttcttaccc |
| 1681 | gtatgcattt tctgtggccc ggcctctgac ttcggattcc aagctggtat ctctggaggt |
| 1741 | gaacaggctc ccctgcactt ccccatccgg tagcaccacc acccagcctg cacccgatgg |
| 1801 | ggtccctggg cctttggcag atacctccct tgttactgct tctgccaagg tgcttccaac |
| 1861 | tccacagcct ctgctgccag cccccagtgg gagctcagcc ccaccgcacc ccgccaagat |
| 1921 | gcccagtggc accgagcagc aaacagaagg gacttccgtt accttctctc ctcttaagtc |
| 1981 | accgccacag ctggaacgag agatggcctc tccacctgag tgcagcgaga tgccccttga |
| 2041 | tctgtcctcc aagtccaacc gccagaagct tccattgccg aaccagcgca agacaccccc |
| 2101 | catgcctgtg ttgacccccg tgcacaccag cagcaaggcc ctcctctcca cagtcctgtc |
| 2161 | taggtctcag cgcacaaccc aggctgccgg tggcaatgtc acctcctgcc tgggctccac |
| 2221 | ttcctcgccc tttgtcatct ttcccgagat cgtgaggaat ggggacccga gcacctgggt |
| 2281 | gaagaactca actgcactga tcagcaccat tcctggcacc tacgtgggag tggccaaccc |
| 2341 | agtgcctgca tccctgctgc tgaacaaaga ccccaacctg ggcctcaacc gtgacccccg |
| 2401 | ccatctcccc aagcaggagc ccatctccat cattgatcaa ggagagccta agggcactgg |
| 2461 | tgccacgtgt ggcaaaaagg gcagccaggc tggtgctgag ggacagccaa gcacagtgaa |
| 2521 | acgatatact ccagcccgca ttgcccctgg gctgccaggg tgccaaacca aggaactctc |
| 2581 | tttgtggaaa cccacggggc cggcaaatat ttatccccgg tgttcagtca atgggaaacc |
| 2641 | taccagcacc caggtcctgc ctgttggctg gtccccgtac caccaggcgt ctctgctttc |
| 2701 | cattggcatt tccagtgccg ggcagctgac ccccagtcag ggggcgccca tcaggcccac |
| 2761 | cagcgttgtt tcggagtttt ctggtgtgcc atctctcagc tccagcgaag ccgtgcacgg |
| 2821 | acttcctgag gggcaaccac ggcctggggg ctccttcgtt ccagagcagg accctgttac |
| 2881 | aaagaacaaa acttgccgga ttgctgccaa gccttatgaa gaacaagtca atcctgtcct |
| 2941 | cttgaccctc agccctcaga ctgggaccct ggcactgtct gttcagccta gcggtgggga |
| 3001 | cattcgaatg aatcaggggc ctgaggaatc agagagccac ctctgctctg acagcactcc |
| 3061 | taagatggaa ggcccccagg gggcttgtgg cctgaagctg gcaggagaca cgaagcctaa |
| 3121 | gaaccaagtg ctggccacct acatgtccca tgagctggtc ctggccaccc cccagaacct |
| 3181 | gcctaagatg cctgagctgc ctttgctacc tcacgacagc caccccaagg aacttatatt |
| 3241 | ggacgtggtt ccgagcagca ggaggggctc cagcacagag cgcccacagc ttggaagcca |
| 3301 | ggtggatctg gggcgagtga aaatggagaa ggtggatggt gatgtggtct tcaatttagc |
| 3361 | cacctgcttc cgggctgatg gcctcccagt ggctccccag aggggccaag ctgaagttcg |
| 3421 | ggctaaggcc gggcaggctc gagtgaaaca ggaaagcgta ggggtctttg cttgcaagaa |
| 3481 | caagtggcag ccagatgatg tgacggaatc tctgccgccc aagaagatga agtgcggcaa |
| 3541 | agagaaggac agtgaagagc agcagctcca gccacaagcc aaggccgtgg tccggagttc |
| 3601 | ccacagaccc aagtgccgga agctgcccag tgacccccag gaatccacca agaaaagccc |
| 3661 | caggggggct tcagattcag gaaaagagca caatggagtc aggggaaagc acaagcaccg |
| 3721 | gaagccgaca aagccggagt cccagtctcc aggaaaacga gccgacagcc acgaggaagg |
| 3781 | ttccttggaa aagaaagcaa agagcagttt ccgtgacttt attcctgtgg ttctgagcac |
| 3841 | ccgcacgcgc agtcagtctg gaagcatctg tagctccttt gctggcatgg cagacagtga |
| 3901 | catgggaagc caggaagtct tccccacaga agaagaagag gaggtaaccc ccaccccagc |
| 3961 | taagcgtcga aaggtgagaa agacccaacg ggacacccag tatcgcagcc accatgccca |
| 4021 | ggacaagtct ctgctgagcc agggccgaag gcacctgtgg cgagcccgag aaatgccctg |
| 4081 | gaggacagag gctgcccggc aaatgtggga caccaatgag gaggaggagg aagaagagga |
| 4141 | ggagggcctg ctgaagagga agaaacgaag acggcagaag agccgaaaat atcagactgg |
| 4201 | ggagtacctg acagagcaag aagacgagca gcggcggaaa gggagagcag atttaaaggc |
| 4261 | ccgtaagcag aagacttcct cctcccaaag tttggagcac cgcctcagga acaggaacct |
| 4321 | tctcttgccc aacaaagtcc aggggatctc ggattcacca aacggtttcc tcccaaataa |
| 4381 | cctggaagag ccagcctgcc ttgaaaattc agaaaagcca tcaggaaaac gaaagtgcaa |
| 4441 | gaccaagcac atggcaaccg tctcagaaga ggcaaaggat gttgttctct actgcctcca |
| 4501 | gaaagacagt gaagatgtga atcaccgtga caatgctggc tacacagccc tgcatgaggc |
| 4561 | ttgttcccgg ggctggaccg acatcctgaa catcctgctg gagcacgggg ccaacgtgaa |
| 4621 | ctgcagtgcg caggacggca cgaggccagt tcatgatgcg gtggtcaatg acaacctgga |
| 4681 | gaccatctgg ctcctgctgt cctatggggc cgatcccaca ctggctacct actcgggtca |
| 4741 | gacagccatg aagctggcca gcagcgacac catgaagcgc tttctcagtg atcacctctc |
| 4801 | ggatcttcag ggccgggcag agggtgatcc cggtgtatcc tgggattttt acagcagttc |
| 4861 | tgtgttggag gaaaaagacg ggtttgcctg tgacctccta cataatcctc ctgggagctc |
| 4921 | agatcaagaa ggagacgatc cgatggagga ggatgatttc atgtttgaac tctcagacaa |
| 4981 | gcctcttctc ccttgctaca acctccaagt gtcagtgtcc cgcgggccct gcaactggtt |
| 5041 | cctcttttcc gatgtcttga agaggctgaa gctttcctcg aggatctttc aggcccggtt |
| 5101 | cccgcacttt gaaatcacca ccatgcccaa ggccgagttc tacaggcagg tggcctccag |
| 5161 | tcagctgctg acccctgccg agaggcctgg aggcttggac gacagatccc ccccaggctc |
| 5221 | ctctgagact gtggagctgg tgcggtacga gccagaccta cttcggctcc tagggtccga |
| 5281 | ggtggaattc cagtcttgca acagttgacc gggaaaacag cccctcctct tctttctcct |
| 5341 | tccgagttcg cccttccccc acctccttgt ctttccccga ccgagcacca gactgcagaa |
| 5401 | tgaggcaata atacggacca acaagaagcc gccttatcaa tgccagcatt agcgactgga |
| 5461 | ctgtttttgt ttttttggtt acaattagtt ctcatctccc tgtcgtcgtc attgttatcg |
| 5521 | tggttgctga tgggggtgga aagttgaact ccatgtctga ggacaagagg tcccgggggt |
| 5581 | ggtgggaggt ggcgccgggg tcccttggac tggcctcctt gttcatgacc aagaccaaac |
| 5641 | ctgggccctg gatggccttg gcctgtcccg aggagaaatg agaaaatccc agatctctga |
| 5701 | gcgcccccca actccattcc cctgtgttct tctgtcttct gtagtattta ttttattagt |
| 5761 | atttaatttg tattgtttca ttggtttctg ataagtctgt atcactgtga cgatttgaga |
| 5821 | caacttgttg tattgaggga ctttctgtac ctccttttct ttttctttgt tgatgagctc |
| 5881 | tgacaaagct attccctggt gtttttttcc cccactgggg agggggtgag gtggaatggg |
| 5941 | gtgggggaac atggacttgt gactaacgaa gctggttgct gctggcccag ggctgggggc |
| 6001 | ttgggggtaa atcctgaggc tttggtgctc ccccacccac ccattcccgc cctttgcagc |
| 6061 | agccccgcta tcttgagatt agtgttgaca gggaggggag gattgtgagg tgaggggtta |
| 6121 | ataagttact ctaataaagg agcgtggaga agggatctga ggggtgaggg tggcccccct |
| 6181 | cctcacgcct tcttcactgc ccccctcaga gtgcacaata cgagtttgtt cctgcctcca |
| 6241 | ctctcccacc ccgttctggc ctccctgtct caagatactg agcctctcac ctcccagccc |
| 6301 | tcagccaccc ccatccctgc cccttctgag actcacagca cccctttcct tcctctcctc |
| 6361 | ccacctcctc cctcagcccc tcattctcct tgggaatctg cagagggctc tgggactcac |
| 6421 | tgccggatgt gaaatccagg cgtcagctgt ttcctaggca agggcaggaa agtggtctcc |
| 6481 | agcccttgct ccactcatgc ctgggggcct ggggctgagt ggtatcccta cctggcctcc |
| 6541 | ccctggcctc tgggcctcca gcgctgggtt tgtcgagtga gagagagaga ggagcttggg |
| 6601 | ttgcttccct gtccccgccc cctctgtggc attgtccctc ccactcttat ttttctacca |
| 6661 | attgctattt ttccgaacaa tccttgtaga gtatgtacca tccaaaggca ggagggcctc |
| 6721 | gccgtggccg gctctggttg gagatggtac agttttattg tacaggtgct aaaacaacaa |
| 6781 | caacaaaaaa gaaaatggaa aaaaaaaaga ttaaaaaaaa aaggaaaaaa aaaaagccag |
| 6841 | tttgaggatg ggacaatctg ttctctagag gctcctgagc catgcgggag cattggtggt |
| 6901 | tattttcttt gtattgtgtt tgttctttgt tcctgggggg gaagttctcg gcccccttct |
| 6961 | gtaggactgc tccccacccc caccatactg cccagttggt tttgaacagt tgttttccct |
| 7021 | ttttaagaaa aaaaaataca tatatatata catatatata tataaagttg aggggttttg |
| 7081 | gactttaatt tgttggtttt gttggggttc ctggtattgt gtagtttatt tcatgttctg |
| 7141 | tttgcctttc cttttttcgc atttgggtgt atattctggc tgccctttat gtttcatttt |
| 7201 | aagcaactgg ctgtggagtc aaaaacactt gcatactgaa aaa |

SEQ ID NO: 6 Human BCORL1 Isoform 1 (Encoded by Transcript Variant 1) Amino
Acid Sequence (NP_068765.3)

| | |
|---|---|
| 1 | mistaplysg vhnwtssdri rmcgineerr aplsdeestt gdcqhfgsqe fcvsssfskv |
| 61 | eltavgsgsn argadpdgsa teklghksed kpddpqpkmd yagnvaeaeg llvplsspgd |
| 121 | glklpasdsa easnsradcs wtplntqmsk qvdcspagvk aldsrqgvge kntfilatlg |
| 181 | tgvpvegtlp lvttnfsplp apicppapgs asvphsvpda fqvplsvpap vphsglvpvq |
| 241 | vatsvpapsp plapvpalap appsvptlis dsnplsvsas vlvpvpasap psgpvplsap |
| 301 | apaplsvpvs applaliqap vppsaptlvl apvptpvlap mpastppaap appsvpmptp |
| 361 | tpssgppstp tlipafaptp vpaptpapif tpaptpmpaa tpaaiptsap ipasfslsrv |
| 421 | cfpaaqapam qkvplsfqpg tvltpsqplv yipppscgqp isvatlpttl gvsstltlpv |
| 481 | ipsylqdrcl pgvlaspelr sypyafsvar pltsdsklvs levnrlpcts psgstttqpa |
| 541 | pdgvpgplad tslvtasakv iptpqpllpa psgssapphp akmpsgteqq tegtsvtfsp |
| 601 | lksppqlere masppecsem pldlssksnr qklplpnqrk tppmpvltpv htsskallst |
| 661 | vlsrsqrttq aaggnvtscl gstsspfvif peivrngdps twvknstali stipgtyvgv |
| 721 | anpvpaslll nkdpnlglnr dprhlpkqep isiidqgepk gtgatcgkkg sqagaegqps |
| 781 | tvkrytpari apglpgcqtk elslwkptgp aniyprcsvn gkptstqvlp vgwspyhqas |
| 841 | llsigissag qltpsqgapi rptsvvsefs gvpslsssea vhglpegqpr pggsfvpeqd |
| 901 | pvtknktcri aakpyeeqvn pvlltlspqt gtlalsvqps ggdirmnqgp eeseshicsd |
| 961 | stpkmegpqg acglklagdt kpknqvlaty mshelvlatp qnlpkmpelp llphdshpke |
| 1021 | lildvvpssr rgssterpql gsqvdlgrvk mekvdgdvvf nlatcfradg lpvapqrgqa |
| 1081 | evrakagqar vkqesvgvfa cknkwqpddv teslppkkmk cgkekdseeq qlqpqakavv |
| 1141 | rsshrpkcrk lpsdpqestk ksprgasdsg kehngvrgkh khrkptkpes qspgkradsh |
| 1201 | eegslekkak ssfrdfipvv lstrtrsqsg sicssfagma dsdmgsqevf pteeeeevtp |
| 1261 | tpakrrkvrk tqrdtqyrsh haqdksllsq grrhlwrare mpwrteaarq mwdtneeeee |
| 1321 | eeeegllkrk krrrqksrky qtgeylteqe deqrrkgrad ikarkqktss sqslehrlrn |
| 1381 | rnlllpnkvq gisdspngfl pnnleepacl ensekpsgkr kcktkhmatv seeakdvvly |
| 1441 | clqkdsedvn hrdnagytal heacsrgwtd ilnillehga nvncsaqdgt rpvhdavvnd |
| 1501 | nletiwllls ygadptlaty sgqtamklas sdtmkrfisd hlsdlqgrae gdpgvswdfy |
| 1561 | sssvleekdg facdllhnpp gssdqegddp meeddfmfel sdkpllpcyn iqvsvsrgpc |
| 1621 | nwflfsdvlk rlklssrifq arfphfeitt mpkaefyrqv assqlltpae rpggiddrsp |
| 1681 | pgssetvelv ryepdllrll gsevefqscn s |

SEQ ID NO: 7 Human RING1A cDNA sequence (BC051866.2, CDS region from
position 188-1408)

| | |
|---|---|
| 1 | cgggccatgg cggcggcggt ggcgggagct gctgtctgag cagcggttgc ggaccgagcg |
| 61 | aacttggccc aggagcccgg gcctagggag aggcgcggcg gcggcgggag cgcgaacggc |
| 121 | tggagctggc cttcttcgcc ttctcctcgg ctgtggagcc ctggtggggg gtctgcgccc |
| 181 | ggtcaccatg acgacgccgg cgaatgccca gaatgccagc aaaacgtggg aactgagtct |
| 241 | gtatgagctg caccggaccc cgcaggaagc cataatggat ggcacagaga ttgctgtttc |
| 301 | ccctcggtca ctgcattcag aactcatgtg ccctatctgc ctggacatgc tgaagaatac |
| 361 | gatgaccacc aaggagtgcc tccacagatt ctgctctgac tgcattgtca cagccctacg |
| 421 | gagcgggaac aaggagtgtc ctacctgccg aaagaagctg gtgtccaagc gatccctacg |
| 481 | gccagacccc aactttgatg ccctgatctc taagatctat cctagccggg aggaatacga |
| 541 | ggcccatcaa gaccgagtgc ttatccgcct gagccgcctg cacaaccagc aggcattgag |
| 601 | ctccagcatt gaggaggggc tacgcatgca ggccatgcac agggcccagc gtgtgaggcg |
| 661 | gccgatacca gggtcagatc agaccacaac gatgagtggg ggggaaggag agcccgggga |
| 721 | gggagaaggg gatggagaag atgtgagctc agactccgcc cctgactctg ccccaggccc |
| 781 | tgctcccaag cgaccccgtg gagggggcgc aggggggagc agtgtaggga cagggggagg |
| 841 | cggcactggt ggggtgggtg ggggtgccgg ttcggaagac tctggtgacc ggggagggac |
| 901 | tctgggaggg ggaacgctgg gccccccaag ccctcctggg gcccccagcc ccccagagcc |
| 961 | aggtggagaa attgagctcg tgttccggcc ccaccccctg ctcgtggaga agggagaata |
| 1021 | ctgccagacg aggtatgtga agacaactgg gaatgccaca gtggaccacc tctccaagta |
| 1081 | cttggccctg cgcattgccc tcgagcggag gcaacagcag gaagcagggg agccaggagg |
| 1141 | gcctggaggg ggcgcctctg acaccggagg acctgatggg tgtggcgggg agggtggggg |
| 1201 | tgccggagga ggtgacggtc ctgaggagcc tgctttgccc agcctggagg gcgtcagtga |
| 1261 | aaagcagtac accatctaca tcgcacctgg aggcggggcg ttcacgacgt tgaatggctc |
| 1321 | gctgaccctg gagctggtga atgagaaatt ctggaaggtg tcccggccac tggagctgtg |
| 1381 | ctatgctccc accaaggatc caaagtgacc ccaccagggg acagccagag gaaggggacc |
| 1441 | atggggtatc cctgtgtcct ggtctatcac cccagcttct ttgtccccca gtacccccag |
| 1501 | cccagccagc caataagagg acacaaatga ggacacgtgg cttttataca aagtatctat |
| 1561 | atgagattct tctatattgt acagagtggg gcaaaacacg cccccatctg ctgccttttc |
| 1621 | tattgccctg caacgtccca tctatacgag gtgttggaga aggtgaagaa ccctcccatt |
| 1681 | cacgcccgcc taccaacaac aaacgtgctt ttttcctctt tgaaaaaaaa aaaaaaaaa |

SEQ ID NO: 8 Human RING1A Amino Acid Sequence (CAI95620.1)

| | |
|---|---|
| 1 | mttpanaqna sktwelslye lhrtpqeaim dgteiavspr slhselmcpi cldmlkntmt |
| 61 | tkeclhrfcs dcivtalrsg nkecptcrkk lvskrslrpd pnfdaliski ypsreeyeah |
| 121 | qdrvlirlsr lhnqqalsss ieeglrmqam hraqrvrrpi pgsdqtttms ggegepgege |
| 181 | gdgedvssds apdsapgpap krprgggagg ssvgtggggt ggvgggagse dsgdrggtig |
| 241 | ggtlgppspp gapsppepgg eielvfrphp llvekgeycq tryvkttgna tvdhiskyla |
| 301 | lrialerrqq qeagepggpg ggasdtggpd gcggegggag ggdgpeepal pslegvsekq |
| 361 | ytiyiapggg afttlngslt lelvnekfwk vsrplelcya ptkdpk |

SEQ ID NO: 9 Human RING1B cDNA Seauence (NM_007212.4, CDS from position 95-1105)

| | |
|---|---|
| 1 | atattgtgcg gcggcgccgg cgtccgcggc agctgatacc agagtcttgc tccggccgcg |
| 61 | gccagcggag ccctgggctg gggcaggagc cgcaatgtct caggctgtgc agacaaacgg |
| 121 | aactcaacca ttaagcaaaa catgggaact cagtttatat gagttacaac gaacacctca |
| 181 | ggaggcaata acagatggct tagaaattgt ggtttcacct cgaagtctac acagtgaatt |
| 241 | aatgtgccca atttgtttgg atatgttgaa gaacaccatg actacaaagg agtgtttaca |
| 301 | tcgtttttgt gcagactgca tcatcacagc ccttagaagt ggcaacaaag aatgtcctac |
| 361 | ctgtcggaaa aaactagttt ccaaaagatc actaaggcca gacccaaact ttgatgcact |
| 421 | catcagcaaa atttatccaa gtcgtgatga gtatgaagct catcaagaga gagtattagc |
| 481 | caggatcaac aagcacaata atcagcaagc actcagtcac agcattgagg aaggactgaa |
| 541 | gatacaggcc atgaacagac tgcagcgagg caagaaacaa cagattgaaa atggtagtgg |
| 601 | agcagaagat aatggtgaca gttcacactg cagtaatgca tccacacata gcaatcagga |
| 661 | agcaggccct agtaacaaac ggaccaaaac atctgatgat tctgggctag agcttgataa |
| 721 | taacaatgca gcaatggcaa ttgatccagt aatggatggt gctagtgaaa ttgaattagt |
| 781 | attcaggcct catcccacac ttatggaaaa agatgacagt gcacagacga gatacataaa |
| 841 | gacttctggt aacgccactg ttgatcactt atccaagtat ctggctgtga ggttagcttt |
| 901 | agaagaactt cgaagcaaag gtgaatcaaa ccagatgaac cttgatacag ccagtgagaa |
| 961 | gcagtatacc atttatatag caacagccag tggccagttc actgtattaa atggctcttt |
| 1021 | ttctttggaa ttggtcagtg agaaatactg gaaagtgaac aaacccatgg aactttatta |
| 1081 | cgcacctaca aaggagcaca aatgagcctt taaaaaccaa ttctgagact gaactttttt |
| 1141 | atagcctatt tctttaatat taaagatgta ctggcattac ttttatggac agatcttgga |
| 1201 | tatgttgttc aattttcttt ctgagccaga ctagtttacg ctattcaaat cttttcccct |
| 1261 | ttatttaaga tttccttttt ggaagggact gcaattattc agtatttttt tctttccttt |
| 1321 | aaaaaaatat atctgaagtt tcttgtgttt ttttttttcc ccacaaagtg tgtttccact |
| 1381 | tggagcacca ttttgaccca ggaatttttc atagtttctg tattcttata agattcagtt |
| 1441 | ggctgtcctt ttcctgctcc cctcaaaaga tttttagtca tacagaatgt taaatattat |
| 1501 | gtattctgac tttttttttc ccccggagtc ttgtatattt atagttttct atataaactg |
| 1561 | tagtatcttc atgaagaccc aaggctcaaa tttactgtcc ttaaaaacaa ttctcatagg |
| 1621 | attattcttt tcatggtatt ttcttccata atatctcatt ttaaaaagaa gttctttatg |
| 1681 | aacttagtgt ccattgtcat gcaatgtttt tttttttcca ttctttttcc ctgtaatttt |
| 1741 | ggaatttctg gtcctgggaa gaatcaaaca aaatcttaag ttctatgaga acttggttca |
| 1801 | ttgacatatt ctgctgaaga aagaaaaatt aaattggtag taaaatatag tcttcaagta |
| 1861 | tacgtttgag agtgcttttt tttgtattag ttctgctgtc acttcatttc ctgtattata |
| 1921 | tgtgatgttt ttccccatta aaataccaga gataatggag atattttgca ctttagcctt |
| 1981 | gatgaaaagt acaagatatg ttcaaagctt ccctaatttt tttcttattt gtagccacat |
| 2041 | aagtttcaag aataacatgg cacacagaac aatggaaaaa agtttgtttc cattggaaaa |
| 2101 | ttatatcatt ttgggttgcc acatcagttt ataaatttgg cgctctttta attacactct |
| 2161 | gtagaaggtt aatagagctt gagccctgct ttaatatgta gtgaaagata attctgtaga |
| 2221 | aaaacgtcag ccagtagggt aaagtcattc tactgttctt aatttttata ttgaggaaca |
| 2281 | atattgggtg tttgggagcc agaaagcttt gttgacagat cagaaataag attgacttgg |
| 2341 | gtgttatatt tcatctctct ccagactcta ggtatatttc caactttata tatcacagta |
| 2401 | tttaaaaaga catgtttgca ttgagaaatt aaccctaaag ggttttcaat agggtgtaga |
| 2461 | cctccagtac ctttgtaact aaagtctgtc tagtcattgt aaatatttat ctgtcagttt |
| 2521 | tgacagattg gggccagctt gatgttttaa atcttcagcc cggtatgaaa acttaaaggt |
| 2581 | atatattcaa ttttttacca ttttatggaa aatatttaaa atctgttttt acagggtttt |
| 2641 | tttttttttt ttttttttgt aatctgtgcc atgaaatttg aaaaccacca aaaatcaagg |
| 2701 | gaacttttat atattcaatt ccttttctgg tgtaatgtta aagttgtata gattattaat |
| 2761 | gcatgcccac tgaatataac cctggttttg tgataaaact gcttagattt tgttgatgac |
| 2821 | attagattag tagttgcatt aaataactaa attcccattg tgattaattg aaattttgtc |
| 2881 | tttaagcaga gagttatttg tgactataag ctttgtgctt agagaatgta tgtgttttta |
| 2941 | tctgtcagta tgggaggata taaactgcat cattagtgaa attattggtt gtgtaatcct |
| 3001 | ttgtgaaata taattctagg tatttgatag ggtattgagt gtattttgtg tgtgtgtgga |
| 3061 | tgtgtgtttt ggggtacggg gagaggcgat gctattggcc atcactacca accagggttt |
| 3121 | caaaaagtat tacctaagta atttctttta tcactatctc aactgaggaa gaaaaggctc |
| 3181 | accacaagtg gtgtgaaggc tttgggtact tagttctaaa tttttttatg gtaacatata |
| 3241 | catagccaca tttacagttt taaccatttt aaggcatgta attcagtggg gttaggtaca |
| 3301 | ttcacaatgt tgtgtaatga tcaccgctgt ctacttgtaa aactttttca tcaccccaaa |
| 3361 | cagaaactct gtgtgcaatt aaagtaatgc atttctcttc ttcttaa |

SEQ ID NO: 10 Human RING1B Amino Acid Sequence (NP_009143.1)

| | |
|---|---|
| 1 | msqavqtngt qplsktwels lyelqrtpqe aitdgleivv sprslhselm cpicldmlkn |
| 61 | tmttkeclhr fcadciital rsgnkecptc rkklvskrsl rpdpnfdali skiypsrdey |
| 121 | eahqervlar inkhnnqqal shsieeglki qamnrlqrgk kqqiengsga edngdsshcs |
| 181 | nasthsnqea gpsnkrtkts ddsgleldnn naamaidpvm dgaseielvf rphptimekd |
| 241 | dsaqtryikt sgnatvdhls kylavrlale elrskgesnq mnldtasekq ytiyiatasg |
| 301 | qftvlngsfs lelvsekywk vnkpmelyya ptkehk |

SEQ ID NO: 11 RYBP cDNA Sequence (NM_012234.6, CDS region from position 184-870)

| | |
|---|---|
| 1 | agtctcgtcc ggagactggc agcggcggcg gcggcggcgg ccggagctcg agccccagcg |
| 61 | gctgagggcg ggcgggcggg cgcgggggag ggaggggggc cggtccgcga cgactccccg |
| 121 | gacggcgttt ctcctccgag cggcgccggt ttcggcttgg ggggggcggg gtacagccca |
| 181 | tccatgacca tgggcgacaa gaagagcccg accaggccaa aaagacaagc gaaacctgcc |
| 241 | gcagacgaag ggttttggga ttgtagcgtc tgcaccttca gaaacagtgc tgaagccttt |
| 301 | aaatgcagca tctgcgatgt gaggaaaggc acctccacca gaaaacctcg gatcaattct |
| 361 | cagctggtgg cacaacaagt ggcacaacag tatgccaccc caccaccccc taaaaaggag |
| 421 | aagaaggaga aagttgaaaa gcaggacaaa gagaaacctg agaaagacaa ggaaattagt |
| 481 | cctagtgtta ccaagaaaaa taccaacaag aaaaccaaac caaagtctga cattctgaaa |
| 541 | gatcctccta gtgaagcaaa cagcatacag tctgcaaatg ctacaacaaa gaccagcgaa |
| 601 | acaaatcaca cctcaaggcc ccggctgaaa aacgtggaca ggagcactgc acagcagttg |
| 661 | gcagtaactg tgggcaacgt caccgtcatt atcacagact ttaaggaaaa gactcgctcc |
| 721 | tcatcgacat cctcatccac agtgacctcc agtgcagggt cagaacagca gaaccagagc |
| 781 | agctcggggt cagagagcac agacaagggc tcctcccgtt cctccacgcc aaagggcgac |
| 841 | atgtcagcag tcaatgatga atctttctga aattgcacat ggaattgtga aaactatgaa |
| 901 | tcagggtatg aaattcaaaa cctccacctg cccatgctgc ttgcatccct ggagaatctt |
| 961 | ctgtggacat cgacctctta gtgatgctgc caggataatt tctgcttgcc atgggcatct |
| 1021 | ggccaccaag gaatttcgca ccctgacgat tactcttgac acttttatgt attccattgt |
| 1081 | tttatatgat tttcctaaca atcatttata attggatgtg ctcctgaatc tactttttat |
| 1141 | aaaaaaaaaa aaatctgctg tgcacaattt tccatgtaca ttacaactgg ttttttgttt |
| 1201 | ttgttttgtt gccggtgggg agggctggga gggggaggga acttttattt attgtgttca |
| 1261 | caaactccat cctttcagca tatcctttta agtttagttc tttcttccag ttatactatg |
| 1321 | tactatcagt tttgatataa ctatatatat ataaatataa aattatatat aaagggttat |
| 1381 | ttgaaaccaa tccatggcaa cgctggtgct tgatacactg tgaagtgaat acaacattga |
| 1441 | acagttacag atctgggaca gtcccttcta tgaaagtgct gaaatttaat taaaatcagt |
| 1501 | cttatatgaa gtatgttcca atccatgtgg gaacttgact ctctcatctg tctaaagagt |
| 1561 | actggacgat ataaaaatat atatttttta aacaatgtga tctcaaattt aaagactgct |
| 1621 | ccagatagcc tgcatttgca atggaataac tgacaaatca caagtggttt agttgggcag |
| 1681 | ggctttgatc attcaaaagt aactaaagta gctccagaat gccaagtatt cgtgtaaatt |
| 1741 | acggttacat gttatcattt gctgttctta cataagcact catgaaaata tggtattctg |
| 1801 | taacttgaat tccatccatt ttccagacct ctactcatgt ctgaggtaaa tctagaaatt |
| 1861 | gtcttagttt taggattgaa acagtctata aactgtattt ttggtccatc caggaagcta |
| 1921 | gtcccttgtt tctcctttct acatgacatt gcagtggtgg tttctgtaat taaaatttgt |
| 1981 | ttgcctcatg tccctttgtc tgataaacct tcactctacc gattcagttg tgagcattct |
| 2041 | ttttttcctt ctcaaaacct actatgattt gttttactga acaaaggtta tcaaccacac |
| 2101 | atccagtcct gacatggagc ttttcagtgt ttggagacat ttctcaatcc cctgctgtgg |
| 2161 | taggaactcc agtggtgaac ggcttgcgcg cctgcagcca gagttgcagg gaaagctcgt |
| 2221 | acttactgcg agcagcatgt aatctttttt cttcctggac ataaagatag cttgagtaaa |
| 2281 | ctgttctatt tcattctctt cactcttttt actgtcttgc aaaaaaaaaa aaaaaaaaaa |
| 2341 | taatcaaaga ccactaataa gattccacct ctccttatta aaataatttt ttaaaatttt |
| 2401 | gttttgcttt tgtttggatg tggggtctct cttctatttg acttttacat ttagatacag |
| 2461 | agtttgtagt acttcagaga catttcaagc atgagaattt gaggttacct ctctttattt |
| 2521 | gacctttagg gactcacggg agggcagcct gatttgtaat gaagcaccac attttggtgt |
| 2581 | taaaaacctg gtttgcttaa taatagcagt aatttctgtc tgtggaggca acaaataaaa |
| 2641 | aaattaacag cttgaattga gtagccaaca ggaaaggttc ctttcacatt tacattaaaa |
| 2701 | ctattctgta gtcactaatg taccataatt taaattcttt tctcaaaggt atagattata |
| 2761 | aagcagtgcc atttgttgct gtggtcctat tctcaaatgc atggacaatg ttcccccctt |
| 2821 | tttaaaataa tgcttgtgtc tgggatgcaa gctttgctta tctttttaaa tacattttta |
| 2881 | aagtatttat taatgaacca aaggaaatca gatgctttct ataagcatca gaatatataa |
| 2941 | tacatagtga tttgactatg aattttaaat ccacatttta atattggtgg gatattgcaa |
| 3001 | agacattcct tctaaagttt taatattcct tttattaagg gtctcaggga gggtaaatta |
| 3061 | gtcagccata tttattttcc agaggtttaa gaaattgctg tttttaactt tttgaaaaaa |
| 3121 | cttaaatgcc accaaactca tgtaggttgc actgcttatt gaaccaataa ctgttggtat |
| 3181 | gcactttgtt cagacacact gtgtactttt tcaaaaacta gtttcatgta aagtgattgg |
| 3241 | accccataga ttagtggaaa aagctgatta accagctact cataggctgc taattcattc |
| 3301 | atgccaatgt tttggttttt cagttttgcc tccgtgataa attaaagaat ggggaggggt |
| 3361 | gaaggaaggg gaagaagatt gctttagaac aagtggcatg aaattaccat ctttgtagaa |
| 3421 | accgcagcta acagtgggag ttatctaagc aatcagatgt tacagggcca gccctttagc |
| 3481 | tgctgtggtg tattctgttg ggtagtgagg tagtaggtac tttatagact tttaattttg |
| 3541 | gaaattgatg acatccctca ggcatgtatt ctggaaatgg aattcctgta acttcctgtg |
| 3601 | tctgcagtat gccctacaat tagtaggcag cgtgtaaaaa cactagtgta gattataaag |
| 3661 | atatacatta aaagaggacc agaaatactt ggtattcagt ggcacagaaa gcaggttaaa |
| 3721 | caaacaaaaa gcacagtgtt acgcttgcaa gtttccattt gttttaatac cacgcaatct |
| 3781 | ttcacactcg tgcgtgtgcg cgcacacaga gcttacctga cttgctctgc ttgagtcatg |
| 3841 | cagttacaaa aaaaaagaca tcttgacacc cacacaatat tctaatcaaa acctttcagt |
| 3901 | ttcaatctgg atatttaaaa acattggcag aagcttctgt gagtttagtt ccactaagat |
| 3961 | gtttcacctg ccttatcaag accattctca gtctactttt ttaagctacc gtatcttaaa |
| 4021 | ttattgaaaa tttattaatt gctgaatata taataacctt tgcttgtatg taaccgaaaa |
| 4081 | tggtttaaga gccaacattt agagtatgac aatggagctg aacagttttt aatgcgcaag |
| 4141 | cagttctgtt cttgtgtatg acttgtaacc ttaatttact gtgtaaagat ggttacatta |
| 4201 | tttccttagc tttgtttgtt ggagacaaat agagaatgct tgttaagtat gtcaaaacaa |
| 4261 | tcttatcttg tgaatttttg ttaatgtatt atacgagcta tatttttcat ttgcccagaa |
| 4321 | agacagcttg tataacgctt ttggaagttt ctgctctgta atgtctttag agctgacagt |
| 4381 | ctgttaggtt tgtttttttc ttcatgctaa agtgtcagtt ggtggttttg tgaactggtc |
| 4441 | aaaaattcac aggtcttaaa tgttttgggg gaaatttata ttggacactg ctctttgtct |
| 4501 | agcaaataaa agatgttaat atattcctgt tactggcatg tgcacgacta tgttattaga |
| 4561 | agccacttta tcattttcct gctttaaata gaaatgtcta tttatgaatt ctgcttgtag |
| 4621 | ttttttcaca aataaaatag taaaatttcc attggaaatc ttaaaaaaaa aaaaaaaa |

SEQ ID NO: 12 RYBP Amino Acid Sequence (NP_036366.3)

| | |
|---|---|
| 1 | mtmgdkkspt rpkrqakpaa degfwdcsvc tfrnsaeafk csicdvrkgt strkprinsq |
| 61 | lvaqqvaqqy atppppkkek kekvekqdke kpekdkeisp svtkkntnkk tkpksdilkd |
| 121 | ppseansiqs anattktset nhtsrprlkn vdrstaqqla vtvgnvtvii tdfkektrss |
| 181 | stssstvtss agseqqnqss sgsestdkgs srsstpkgdm savndesf |

SEQ ID NO: 13 Human PCGF1 cDNA Sequence (NM_032673.3, CDS region from position
28-807

| | |
|---|---|
| 1 | agtggccggc tgggatcagc ctttaagatg gcgtctcctc aggggggcca gattgcgatc |
| 61 | gcgatgaggc ttcggaacca gctccagtca gtgtacaaga tggacccgct acggaacgag |
| 121 | gaggaggttc gagtgaagat caaagacttg aatgaacaca ttgtttgctg cctatgcgcc |
| 181 | ggctacttcg tggatgccac caccatcaca gagtgtcttc atactttctg caagagttgt |
| 241 | attgtgaagt acctccaaac tagcaagtac tgccccatgt gcaacattaa gatccacgag |
| 301 | acacagccac tgctcaacct caaactggac cgggtcatgc aggacatcgt gtataagctg |
| 361 | gtgcctggct tgcaagacag tgaagagaaa cggattcggg aattctacca gtcccgaggt |
| 421 | ttggaccggg tcacccagcc cactggggaa gagccagcac tgagcaacct cggcctcccc |
| 481 | ttcagcagct ttgaccactc taaagcccac tactatcgct atgatgagca gttgaacctg |
| 541 | tgcctggagc ggctgagttc tggcaaagac aagaataaaa gcgtcctgca gaacaagtat |
| 601 | gtccgatgtt ctgttagagc tgaggtacgc catctccgga gggtcctgtg tcaccgcttg |
| 661 | atgctaaacc ctcagcatgt gcagctcctt tttgacaatg aagttctccc tgatcacatg |
| 721 | acaatgaagc agatatggct ctcccgctgg ttcggcaagc catccccttt gcttttacaa |
| 781 | tacagtgtga aagagaagag gaggtagggg ccaagccccc accccatccc actccccttc |
| 841 | cctccccaga tatttatgtg aaatgaactg cagctttatt ttttgaaata aaaactttta |
| 901 | aaaagca |

SEQ ID NO: 14 Human PCGF1 Amino Acid Sequence (NP_116062.2)

| | |
|---|---|
| 1 | maspqggqia iamrlrnqlq svykmdplrn eeevrvkikd lnehivcclc agyfvdatti |
| 61 | teclhtfcks civkylqtsk ycpmcnikih etqpllnlkl drvmqdivyk ivpglqdsee |
| 121 | krirefyqsr gldrvtqptg eepalsnlgl pfssfdhska hyyrydeqln lclerlssgk |
| 181 | dknksvlqnk yvrcsvraev rhlrrvlchr lmlnpqhvql ifdnevlpdh mtmkqiwlsr |
| 241 | wfgkpsplll qysvkekrr |

SEQ ID NO: 115 Human SKP1 Transcript Variant 2 cDNA Sequence (NM_170679.3,
CDS region from position 97-588)

| | |
|---|---|
| 1 | gctgtagtgg cttcgtcttc ggtttttctc ttccttcgct aacgcctccc ggctctcgtc |
| 61 | agcctcccgc cggccgtctc cttaacaccg aacaccatgc cttcaattaa gttgcagagt |
| 121 | tctgatggag agatatttga agttgatgtg gaaattgcca aacaatctgt gactattaag |
| 181 | accatgttgg aagatttggg aatggatgat gaaggagatg atgacccagt tcctctacca |
| 241 | aatgtgaatg cagcaatatt aaaaaaggtc attcagtggt gcacccacca caaggatgac |
| 301 | cctcctcctc ctgaagatga tgagaacaaa gaaaagcgaa cagatgatat ccctgtttgg |
| 361 | gaccaagaat tcctgaaagt tgaccaagga acactttttg aactcattct ggctgcaaac |
| 421 | tacttagaca tcaaaggttt gcttgatgtt acatgcaaga ctgttgccaa tatgatcaag |
| 481 | gggaaaactc ctgaggagat tcgcaagacc ttcaatatca aaaatgactt tactgaagag |
| 541 | gaggaagccc aggtacgcaa agagaaccag tggtgtgaag agaagtgaaa tgttgtgcct |
| 601 | gacactgtaa cactgtaagg attgttccaa atactagttg cactgctctg tttataattg |
| 661 | ttaatattag acaaacagta gacaaatgca gcagcaagtc aattgtatta gcagaatatt |
| 721 | gtcctcattg catgtgtagt ttgagcacag atcccaaacc ttacggccaa gtttcttcta |
| 781 | gtatgatgga aagtttcttt tttctttgct ctgaataaaa ctgaactgtg ggttctctat |
| 841 | aagtggcatt ttgggctttc cctctttttt gtaaagcaat gtctgcctag tttattgtcc |
| 901 | agttaacttt agtgaccttt taaaagttgg cattgtaaat aaaacaactt gcaaaaaagt |
| 961 | tttctggaat agaattaaca aaatattatc tttattcatg agttggaaac tggaaaaagg |
| 1021 | cttcttgaag taaatgttct gagtggagct actaggatgt cttccagcct cctgcagtca |
| 1081 | aggagtacca ctgtattgat tagcctgtat gtagcagggc tcccttcatt gcatctgagg |
| 1141 | acttgttttc tttttcttta tttttaatcc tcttagtttt aaatatattg cctagagact |
| 1201 | cagttactac ccagtttgtg gttttttggg agaaatgtaa ctggacagtt agcttttcaa |
| 1261 | ttaaaaagac acttaaccca tgtgggatgt catcttttta taattagtgt tcccatgtgg |
| 1321 | agaaaattat tcacactact tgcatgtaaa gaataattta acttttaaca ttaaaatatg |
| 1381 | tggtaaaacc cagaaagcat ccatcatgaa tgcaagatac tttcaataaa aagtaagtta |
| 1441 | tatagtaggt agttaagttt gcttttgtgg acttaaatgt gtctcttcac ttaaatgggt |
| 1501 | tgaatgtgta tatatttgtt cagcttgaaa agacttagtt tatatcctag ctcactggag |
| 1561 | gctgctgaca taaccataac ttctgtccct tctaattgtc atttatatgc ctaactggag |
| 1621 | ctagtacttt aattcttaac acaaaattac tctgccattg tttccagctt ccctcctaca |
| 1681 | atagaatgaa gtttttttga tggcttgaga tggctcacaa attttgattt ttttttcttc |
| 1741 | cttgtgctcc ctttttttct ccttgctttt ccagttaaca tctatattca catgtaatct |
| 1801 | tgttttctct tcacattcac tgagttgttc aggctcagat catcccttga cagtagtttg |
| 1861 | ccttcatctc acctttcatt tgtcccaaat tcaccttatt taataaagtc ccatatgttg |
| 1921 | tctcacttaa ttccaatttg ttgtctgtac cagagatagg aaactataat gtgtgggtca |
| 1981 | aatcaggccc atagcctgtt tttgtagtct ttgagctaaa aatggttttt acgttgtaaa |
| 2041 | gagttattta aaattctctc tctctctctc tctctctctc aggagataag cttgttttct |
| 2101 | gtttgtcctg gttttagtgg ggaaggtagc ggtgtatagt cctagctgaa ttgtctcatc |
| 2161 | taccaattcc tgctattata agatcaactt ctgcaaaaac cttatccacc tcaagtatcc |
| 2221 | ttagcagctg ggcatggtgg cttatgcctg taatctcaac attttgggag gacaagacag |
| 2281 | gaggatcgtt tgagcccagg agttcaagac cagcctgggc aacacagaaa gtccctgtct |
| 2341 | ctccaaaaaa aaaaaaaaaa attagcagtc atatggtgca tgcctataat cccaactact |
| 2401 | tgagaggctg aggtgggaag atggcttgag cctgagaggt cgaggctgca gtgagctggc |
| 2461 | attgcaccac tgcactccag tctaggcaat agagcgagac cttgtctcaa aaacaaacca |
| 2521 | gaagtatcct tagcagtatt atgacaaaca aatcttcact gaaggtccag gattactgcc |
| 2581 | atatgccatg tttaacttgt aaaggaagta tcaccttcta agaaactgga gcttgttatt |
| 2641 | accacttgat ctgtataata ctcagcagta gttagaatta tatgagtaaa taacgtcact |
| 2701 | atccgtattt aaaggataat gagatctttc agaaagattg gatggacaga caatatagta |
| 2761 | gtaatttaaa tttttgtttc ctgactttct gtagtcccta aacaaacaac aaaaaatcct |
| 2821 | agtaatctta aacttttaca ttaatagaga tccaagagaa aataagccat ttttcaccat |
| 2881 | tgtggaccca aataaatcat agacatggta ttaagaagcc cttttcagtc tggttgcagt |
| 2941 | attattttcc tacctttctt tttcctgttc cctgctgtat gcccactatt cctttaatat |
| 3001 | atctactaga acttttctag gctttcacct ggagtgagtg gtcctttctc attatcacag |
| 3061 | tagccaaacc tcagtcttca aaactccact catttctggc tactctctta cttcccaact |
| 3121 | attcttctaa actaatattg tggcattaag tcataccttg aggctggagt tttagtgtct |
| 3181 | tcatggcctt gggcaagttg gaaataggta ctacggtttt atagatctag tgcagtctgc |
| 3241 | tttatattag ttccagaact tcattggaag tcacttgaca gcaggattta acttgtattt |
| 3301 | agcagcacta ctccatgaat ttcagtataa gtaacagaag tgaaaagtcc ttgtgaaatg |
| 3361 | caggtacagg gcataatgaa aacaagaaga gtagttttaa tgcatgagag gggagagctt |
| 3421 | gataatagct gataatagcc ttggtttgga gagtggttac tgatgaatta tgaagacttt |
| 3481 | ctctaattac tgttatagta gtaaaggaaa gaaaaccctt gttgataaag taaacttagg |
| 3541 | gattaattag gaaatgcctt ttatttcacc aggaaagaga atgagaggaa gaaggtattt |
| 3601 | ggcagatttg ggtgattgaa ggatgtttgt cggtttcctc tgtaaacaac atgctgcttt |
| 3661 | ctgcagtgtg ctgcttttaa tgatgaagtc ctttcaagga ctccatggca gtcctttggc |
| 3721 | ttctcacctt ttcattgatt atgtcacttg tgatctaaaa gtaaaccaaa accttcctga |
| 3781 | atgttagctt attattttct ccttaacatt gtcataggct aaagatgtgc cccctcaaat |
| 3841 | tctaataatt tttttttttt tttttttttt tttgagacag agtctcactc tgtcgcccag |
| 3901 | actggaatgc agtggtgcga tctcagctca ctgcaacctc cgcctcctgg gttcaagtga |
| 3961 | ttctcctgcc tcagcctccc gagtagctgg gattacaggc acatgccacc atgcctggct |
| 4021 | aatttttttg tgtttttatt agagatggag ttttaccata ttggccaggc tggtcttgaa |
| 4081 | ctcctgacct tgtgatccgc ccgccttagc ctccgaaagt gctgaggtta cacgtgtgac |
| 4141 | ccaccgcaca cggccctaat aattcttaag ttgtagaagc aagttactct tttgaagtgc |
| 4201 | catgtgttag cttgccttaa tctattcttg gtatacaaaa tagatactgc tttacagttc |
| 4261 | ttatattttc tcagagattc acaaaatcat cctgtagctc cctgtgtggg attatagctg |
| 4321 | tgacttttta ctctacaact gtcagaatac accagctcct ggaattaagc aagaactata |
| 4381 | ttgtgacttg gttggacagg taactgcagc cttgtaacat gacatcatag taatgctgag |
| 4441 | cttatcttaa aactagctgg ggagggagga tagtaacagt gagtcactgt gggcatctca |
| 4501 | cttagggcag gcagatattc caaatcacat tgatttttca gtttacgtaa aaattgacca |
| 4561 | atcctatggg ctgtgttctg atcccaacat aaattttgtt taagttaatt tgcctaggct |
| 4621 | ctcaataagg cagtttggga atataaggtg attttccacc agaaaagaga aaattgagag |
| 4681 | agcagcagac ctcttgctct cagcaccctt tacaagctta acttttgctt gccaccatta |
| 4741 | gcttttgaag atttttttta actgctcctt gcagagcagg actaccccat aggcagtgtg |
| 4801 | cccagagtag cccgaagagc tttttgattc ttcttttaag agacaaggtc tcacctctgc |
| 4861 | cccccaggct ggagtgcagt gatgtgatca cagctcattg cagcctcgaa ctcttgggct |
| 4921 | ccaacagtcc tcctgcctca gcctcctgaa tagctaggac tacatgtgtg tgctaccatg |
| 4981 | ccttgctagt ttttgttttt ttaaattgat ttttatagag atgaggtctc ctcatgttgc |
| 5041 | ccaggctgtt ttcgaattcc tgggctcaag atcctcctgc ctcaacctcc caaagtgctg |
| 5101 | gtattacaga tgtgagccac agcacccacc aatttttcct tttctaaagc tcagtgtaca |
| 5161 | ttctggtagg aaggaatcaa ccaagtaaat gttctcccta caaagttgtt tgaggatttg |
| 5221 | aaaagttaat cttcaattat ctggatttga aaccagtcta taacttttta agctaggaag |
| 5281 | aagtcaagaa taagaagatt gcaagcaaag ggaaagctta caaaatggct gaatccttaa |
| 5341 | agtgtagctc ctggcttctc cttggtagga agcattagac atggtttccc tctagggagc |
| 5401 | tgggagcttt tggtgtagtt caggagagaa ggcataaata ctgctgggag ggagtggata |
| 5461 | actctaataa aaatgttcct gggctgggca cggtggctca cgcctgtaat cccaacattt |
| 5521 | tcagaggctg aggtgtgcag atcacgaggt caagagatcg agaccatcct ggccaacatg |
| 5581 | gtgaaacctc atctctacta aaaatacaaa aattagctgg gcgtggtggc acatgcctgt |
| 5641 | agtcccagca actcgggagg ctgaggcagg agaaccgctt gaacccggga ggcggaagtt |
| 5701 | gcagtgagcc gacatcacgc cactgactgc agtccagcct ggcgacagag cgagactccg |
| 5761 | tttcaaaaaa aaaaagtttc tggacagaac agattaatga gccaatgggg ggctacagag |
| 5821 | ctgaaaggtg ctacgaaagg ttttgtaaag tgaaactaat ttggtttcaa gaatcaagtt |
| 5881 | ggatttgttt gggggtggac agatgccaaa gtgaggaaaa agcagtgcac agtcaggaac |
| 5941 | taacagcaga aggttgggtg ttggtacatt tccctgagaa tatagactaa agagaaatta |
| 6001 | gggcaggatt agtcacagta ctttgctgac actcagtagt attttatcag catttattca |
| 6061 | tcagacccac attaggcagg gcaatttttg agtgagctaa attctaattt ttttaatttt |
| 6121 | aatttttatt tatttatttt ttggctgttc aactaaacca gtcttttgaa tgttgggggt |
| 6181 | tttacagtta gagtcctgat gactttttct tgtttactgt tgttaaaaat cactacctcc |
| 6241 | agtggtcaca gagaatgagt aagagaactg gctgtgcata tcaagatgaa gtatggtgta |
| 6301 | agaaatattt cagagcatgt cctttggccc aagattgagc catttggaca cctaagcagg |
| 6361 | attggctgaa tgttatcctt tctgctgagg catcagtgga gatgatcatt agtggtggcc |
| 6421 | gaaaagtgtt gtcgaaggtg ctcttgcttg acacacttcc aatgcgggac atagtttgag |
| 6481 | tgatagatgg ctaattcatt gataatgtac ttgttgcctt gcagtcttgt gtactgctgg |
| 6541 | ctacctctgg tctgtgtgcc agtattgagc agcattctat gggaggaatg tagttgctca |
| 6601 | taacagtgtc tgcttccaca tctgtagttc ttgcgacctt ctgatatctg tcaactttaa |
| 6661 | attttctgga agtatttaag tatattcaga ccttctgata tctcttaact tcaaattttc |
| 6721 | aacctgctat atagcagcct cttattgcac actgaggatc agcttatttc cttaaagtgc |
| 6781 | tctcactttg aatacctccc acctatctgg tgaaaattct tatttgtcct ttctttcctg |
| 6841 | aatgtaccat ttcacaacct ctaactgcct tcttcagttg ctttgcagat ttttctgttc |
| 6901 | caggaagctt acggaaaggg gtgggaaaat agagaagctg tactctatcc tcaagtactc |
| 6961 | aagcagggtt tccagagtgg ctttgtaaaa atttttattt atttatttat ttatttttgg |
| 7021 | tgagtggaac ctggcttttc ctcttccctc cccaagcccc accccgcttc ctgacattgc |
| 7081 | acatgcctga aagaactagg cttcagataa aagaatgaga acacttgaat gaaaaaggaa |
| 7141 | gacttggtcc agagcccctg gtgaatatga aagaagtatt ggcctggact atctcccgca |
| 7201 | taattcacag ctgctatctt gtcttcagaa gtgaaacatc ccttaccaat agtgacctaa |
| 7261 | cttgtgagga ctgcttgtga tagtatagtt cccaccccca ggacataagt tttccctttc |
| 7321 | cagttaacat gcctaatccc atgctatctt taagagcttg aaaatccttc ctgacctgag |
| 7381 | ctaggctttt attctaaggg actgtgtatg tgaacatccc caccatctat gtttcctgtt |
| 7441 | ttcaaactcg gtgccgtttt gagtattttc agatgctcac tgccagtaat ggtttgttag |
| 7501 | tagctccaaa ctattagaga agagctcagg agtcagaatt tgaaagatgc ccatagggtg |
| 7561 | atttgagatt tggaacaagg ttatcacaat aagttgggat tgggggagtt gacacaacca |
| 7621 | gggtctgctt gggtgggcag ctgtttcaga aacccttggt gaactcttaa agccctgaag |
| 7681 | agcttaaaaa atgctctgaa gagatctgag agtcgcagtt aaggactctc aaagaacctc |
| 7741 | aaagaaaagc caacttagcc tttttataca attaactaga tttgggtctt aaattctact |
| 7801 | ttgacatata ggcctaagag gtagtttaac atggccttgt tacccttacc ccttctgttc |
| 7861 | tgtgctgttt tctgagaaac ggatgcagga actgattccc tttcttcctc tctggaacag |
| 7921 | gatataaaga acccttccgc ttccctttac ttgtggcctt cctggccagg cttatgtata |
| 7981 | ctgaacaaag ctagggtgca gtgcttctga gctagctgtt aggggtccac agccagaagg |
| 8041 | aatgggagac cacaggagcc attcaaaaga taattacatg tcagaagaga gtccatccat |
| 8101 | ggggttctgt taaagtagac accttcacgt tgggagacat aggctttgtc tgcttgctgg |
| 8161 | gctgaaccct caccatatcc acagagaaca aaatggggag cagctgtacc aggggaggga |
| 8221 | tgaacgtctt tcacccaagt tgccagtctg ctagttgtct ggaaatgttc aagggaggct |
| 8281 | aaggtatctt gtcttatgat gtctcaggaa acagggttgg ttctcagctg gcagtgaagg |
| 8341 | gggactggtg tggtattagc agttgaactt gaggttgcaa tctcaagtca ccctctggcc |
| 8401 | tcgcaggaat gcaccagggt gatttgggag gatctgagtt tcctttatga agtcagatta |
| 8461 | aaaaaaaaaa aagctgatct gatttaaaca gagttgccaa agacttcccc gccaatacac |
| 8521 | aaagactgca ttgttcaggt accctgtgta ctctaagtga gacaccaaat cagatgcagc |
| 8581 | aacctggtct tgcataagcc ctggttagaa tgtaggtttt ttaaacaact aggccagtag |
| 8641 | ccagaacaga attctttaaa atgggacaga tgtccagaga atggctagtg tcctctagca |
| 8701 | ctctctcaga agcaaaagca agtctggggt tacaacttcc agtggcctac tttaaataga |
| 8761 | cgctgttctg gaaaatgact ttatacttta aacacaaaat ccaaaaaata gaactcagtg |
| 8821 | gagtgacaat gacaatgcat gatggtggcc cagtggctgt cccatgtgaa atacttcttg |
| 8881 | cctgacatgt atcatctact tgttgactga cctattgagg tgccttcatg acacctttta |
| 8941 | cattcatgat aggtctcagg aagacagcag tgtacttggt ggaaactcat gtaaaaagtg |
| 9001 | ggttgtaggg agctaaacag tgagtacaca tggtatatgg atacggaatg gaataataga |
| 9061 | cattggagac ttcaaaaggt gggagatgaa agggggatga ggtatgaaat cctacctgtt |
| 9121 | gagtacaatg tacactactt acgtgcacag tacactgttt gggtgagagg cacactaaaa |
| 9181 | gcccggacct caccgctacc cagtatgttc atgtaacaca gctgcacttg taccccctaa |
| 9241 | atgtatacaa ataatctaaa aatagcccgg acctcaccgc tacacaatat attcatgtaa |
| 9301 | cacagctgca tttgtacccc ctaaatgtat acaaataatc taaaaataaa gtggatatgg |
| 9361 | tggctgtcgt gtccaccaac agcgta |

SEQ ID NO: 16 Human SKP1 Isoform B (Encoded by Transcript Variant 2) Amino Acid
Sequence (NP_733779.1)

| | |
|---|---|
| 1 | mpsiklqssd geifevdvei akqsvtiktm ledlgmddeg dddpvplpnv naailkkviq |
| 61 | wcthhkddpp ppeddenkek rtddipvwdq efIkvdqgtl f elilaanyl dikglldvtc |
| 121 | ktvanmikgk tpeeirktfn ikndfteeee aqvrkenqwc eek |

SEQ ID NO: 17 Human MYCL Transcript Variant 1 cDNA Sequence
(NM_001033081.3, CDS region from position 484-1578)

| | |
|---|---|
| 1 | gagtgcgggc cgcgctctcg gcggcgcgca tgtgcgtgtg tgctggctgc cgggctgccc |
| 61 | cgagccggcg gggagccggt ccgctccagg tggcgggcgg ctggagcgag gtgaggctgc |
| 121 | gggtggccag ggcacgggcg cgggtcccgc ggtgcgggct ggctgcaggc tgccttctgg |
| 181 | gcacggcgcg cccccgcccg gccccgccgg gccctgggag ctgcgctccg ggcggcgctg |
| 241 | gcaaagtttg ctttgaactc gctgcccaca gtcgggtccg cgcgctgcga ttggcttccc |
| 301 | ctaccactct gacccggggc ccggcttccc gggacgcgag gactgggcgc aggctgcaag |
| 361 | ctggtggggt tggggaggaa cgagagcccg gcagccgact gtgccgaggg acccggggac |
| 421 | acctccttcg cccggccggc acccggtcag cacgtccccc cttccctccc gcagggagcg |
| 481 | gacatggact acgactcgta ccagcactat ttctacgact atgactgcgg ggaggatttc |
| 541 | taccgctcca cggcgcccag cgaggacatc tggaagaaat tcgagctggt gccatcgccc |
| 601 | cccacgtcgc cgccctgggg cttgggtccc ggcgcagggg acccggcccc cgggattggt |
| 661 | cccccggagc cgtggcccgg agggtgcacc ggagacgaag cggaatcccg gggccactcg |
| 721 | aaaggctggg gcaggaacta cgcctccatc atacgccgtg actgcatgtg gagcggcttc |
| 781 | tcggcccggg aacggctgga gagagctgtg agcgaccggc tcgctcctgg cgcgccccgg |
| 841 | gggaacccgc ccaaggcgtc cgccgccccg gactgcactc ccagcctcga agccggcaac |
| 901 | ccggcgcccg ccgccccctg tccgctgggc gaacccaaga cccaggcctg ctccgggtcc |
| 961 | gagagcccaa gcgactcgga gaatgaagaa attgatgttg tgacagtaga gaagaggcag |
| 1021 | tctctgggta ttcggaagcc ggtcaccatc acggtgcgag cagaccccct ggatccctgc |
| 1081 | atgaagcatt tccacatctc catccatcag caacagcaca actatgctgc ccgttttcct |
| 1141 | ccagaaagct gctcccaaga agaggcttca gagaggggtc cccaagaaga ggttctggag |
| 1201 | agagatgctg caggggaaaa ggaagatgag gaggatgaag agattgtgag tcccccacct |
| 1261 | gtagaaagtg aggctgccca gtcctgccac cccaaacctg tcagttctga tactgaggat |
| 1321 | gtgaccaaga ggaagaatca caacttcctg gagcgcaaga ggcggaatga cctgcgttcg |
| 1381 | cgattcttgg cgctgaggga ccaggtgccc accctggcca gctgctccaa ggcccccaaa |
| 1441 | gtagtgatcc taagcaaggc cttggaatac ttgcaagccc tggtgggggc tgagaagagg |
| 1501 | atggctacag agaaaagaca gctccgatgc cggcagcagc agttgcagaa aagaattgca |
| 1561 | tacctcactg gctactaact gaccaaaaag cctgacagtt ctgtcttacg aagacacaag |
| 1621 | tttatttttt aacctccctc tcccctttag taatttgcac attttggtta tggtgggaca |
| 1681 | gtctggacag tagatcccag aatgcattgc agccggtgca cacacaataa aggcttgcat |
| 1741 | tcttggaaac cttgaaaccc agctctccct cttccctgac tcatgggagt gctgtatgtt |
| 1801 | ctctggcgcc tttggcttcc cagcaggcag ctgactgagg agccttgggg tctgcctagc |
| 1861 | tcactagctc tgaagaaaag gctgacagat gctatgcaac aggtggtgga tgttgtcagg |
| 1921 | ggctccagcc tgcatgaaat ctcacactct gcatgagctt taggctagga aaggatgctc |
| 1981 | ccaactggtg tctctggggt gatgcaagga cagctgggcc tggatgctct ccctgaggct |
| 2041 | cctttttcca gaagacacac gagctgtctt gggtgaagac aagcttgcag acttgatcaa |
| 2101 | cattgaccat tacctcactg tcagacactt tacagtagcc aaggagttgg aaacctttat |
| 2161 | atattatgat gttagctgac ccccttcctc ccactcccaa tgctgcgacc ctgggaacac |
| 2221 | ttaaaaagct tggcctctag attctttgtc tcagagccct ctgggctctc tcctctgagg |
| 2281 | gagggacctt tctttcctca caagggactt ttttgttcca ttatgccttg ttatgcaatg |
| 2341 | ggctctacag caccctttcc cacaggtcag aaatatttcc ccaagacaca gggaaatcgg |
| 2401 | tcctagcctg gggcctgggg atagcttgga gtcctggccc atgaacttga tccctgccca |
| 2461 | ggtgttttcc gaggggcact tgaggcccag tcttttctca aggcaggtgt aagacacctc |
| 2521 | agagggagaa ctgtactgct gcctctttcc cacctgcctc atctcaatcc ttgagcggca |
| 2581 | agtttgaagt tcttctggaa ccatgcaaat ctgtcctcct catgcaattc caaggagctt |
| 2641 | gctggctctg cagccaccct tgggcccctt ccagcctgcc atgaatcaga tatctttccc |
| 2701 | agaatctggg cgtttctgaa gttttgggga gagctgttgg gactcatcca gtgctccaga |
| 2761 | aggtggactt gcttctggtg ggttttaaag gagcctccag gagatatgct tagccaacca |
| 2821 | tgatggattt taccccagct ggactcggca gctccaagtg gaatccacgt gcagcttcta |
| 2881 | gtctgggaaa gtcacccaac ctagcagttg tcatgtgggt aacctcaggc acctctaagc |
| 2941 | ctgtcctgga agaaggacca gcagcccctc cagaactctg cccaggacag caggtgcctg |
| 3001 | ctggctctgg gtttggaagt tggggtgggt agggggtggt aagtactata tatggctctg |
| 3061 | gaaaaccagc tgctacttcc aaatctattg tccataatgg tttctttctg aggttgcttc |
| 3121 | ttggcctcag aggaccccag gggatgtttg gaaatagcct ctctaccctt ctggagcatg |
| 3181 | gtttacaaaa gccagctgac ttctggaatt gtctatggag gacagtttgg gtgtaggtta |
| 3241 | ctgatgtctc aactgaatag cttgtgtttt ataagctgct gttggctatt atgctggggg |
| 3301 | agtctttttt ttttatattg tatttttgta tgccttttgc aaagtggtgt taactgtttt |
| 3361 | tgtacaagga aaaaaactct tggggcaatt tcctgttgca agggtctgat ttattttgaa |
| 3421 | aggcaagttc acctgaaatt ttgtatttag ttgtgattac tgattgcctg attttaaaat |
| 3481 | gttgccttct gggacatctt ctaataaaag atttctcaaa ca |

SEQ ID NO: 18 Human MYCL (Encoded by Transcript Variant 1) Amino Acid
Sequence (NP_001028253.1)

| | |
|---|---|
| 1 | mdydsyqhyf ydydcgedfy rstapsediw kkfelvpspp tsppwglgpg agdpapgigp |
| 61 | pepwpggctg deaesrghsk gwgrnyasii rrdcmwsgfs arerleravs drlapgaprg |
| 121 | nppkasaapd ctpsleagnp apaapcplge pktqaesgse spsdseneei dvvtvekrqs |
| 181 | lgirkpvtit vradpldpcm khfhisihqq qhnyaarfpp escsqeease rgpqeevler |
| 241 | daagekedee deeivspppv eseaaqschp kpvssdtedv tkrknhnfle rkrrndlrsr |
| 301 | flalrdqvpt lascskapkv vilskaleyl qalvgaekrm atekrqlrcr qqqlqkriay |
| 361 | itgy |

SEQ ID NO: 19 BCOR1 Transcript Variant 5 cDNA Sequence (NM_001123385.2; CDS
region from 785-6052)

| | |
|---|---|
| 1 | agacggagcc tgggctccca gcggcaaggt gaggcagagc tgcgctcctc gctgaacgcg |
| 61 | ggccgagctc ggcggctgcg ggggagacgc gcaggagccc agaccgcgac cgagagcggg |
| 121 | agctaggcgg gcggcggcgg cggaggggga gcccgcgagc cgccgggcgg agagcccaag |
| 181 | ccgcgctgtc gccgcgcagg gacgacttgg ccaacactca cacacactca cacacaccca |
| 241 | gcccgagcgg gcgctcgcgg cgaaccgtca acatggcgct ggggctcctg cccgagcgcg |
| 301 | ggcggcggcg gcagcgcggg agctgctgag ctcggccaag cccagtccag ctgcgggagc |
| 361 | ccggaggatc gcacggggct gtcgccacct gcccggaggc cccgagcccg ccccgccccg |
| 421 | cccccacccg gcccagagcc cacccctcgg cggggccgac cccgagggca gccggctgcc |
| 481 | agcagacggc gagggagtcg agtgagcgcg gcgccgcgag cgggctgcgg gcagccgggg |
| 541 | accgcaaact ttgctgctcg ccgcgcttct ccggcccggc tccttctccg ctcgttaacg |
| 601 | tcgccaaccc cccccacccc tcatatctct ctccacccac ccaaccgccc cccgctcctt |
| 661 | ctcgccgcct cgagtccgct tgggggaaaa cttcaaagag ccggatcgca ggctccctgc |
| 721 | ctactccccc accggggatt tcagactaga cgcttgaagc aaagctgcca tcccagaaga |
| 781 | cgacatgctc tcagcaaccc ccctgtatgg gaacgttcac agctggatga acagcgagag |
| 841 | ggtccgcatg tgtggggcga gcgaagacag gaaaatcctt gtaaatgatg gtgacgcttc |
| 901 | aaaagccaga ctggaactga gggaagagaa tcccttgaac cacaacgtgg tggatgcgag |
| 961 | cacggcccat aggatcgatg gcctggcagc actgagcatg gaccgcactg gcctgatccg |
| 1021 | ggaagggctg cgggtcccgg gaaacatcgt ctattctagc ttgtgtggac tgggctcaga |
| 1081 | gaaaggtcgg gaggctgcca caagcactct aggtggcctt gggttttctt cggaaagaaa |
| 1141 | tccagagatg cagttcaaac cgaatacacc cgagacagtg gaggcttctg ccgtctctgg |
| 1201 | aaaaccccca aatggcttca gtgctatata caaaacaccg cctggaatac aaaaaagtgc |
| 1261 | tgtagccaca gcagaagcgc tgggcttgga caggcctgcc agcgacaaac agagccctct |
| 1321 | caacatcaat ggtgctagtt atctgcggct gccctgggtc aatccttaca tggagggtgc |
| 1381 | cacgccagcc atctaccctt tcctcgactc gccaaataag tattcactga acatgtacaa |
| 1441 | ggccttgcta cctcagcagt cctacagctt ggcccagccg ctgtattctc cagtctgcac |
| 1501 | caatggggag cgctttctct acctgccgcc acctcactac gtcggtcccc acatcccatc |
| 1561 | gtccttggca tcacccatga ggctctcgac accttcggcc tccccagcca tcccgcctct |
| 1621 | cgtccattgc gcagacaaaa gcctcccgtg gaagatgggc gtcagccctg ggaatcctgt |
| 1681 | tgattcccac gcctatcctc acatccagaa cagtaagcag cccagggttc cctctgccaa |
| 1741 | ggcggtcacc agtggcctgc cgggggacac agctctcctg ttgcccccct cgcctcggcc |
| 1801 | gtcaccccga gtccaccttc ccacccagcc tgctgcagac acctactcgg agttccacaa |
| 1861 | gcactatgcc aggatctcca cctctccttc agttgccctg tcaaagccat acatgacagt |
| 1921 | tagcagcgag ttccccgcgg ccaggctctc caatggcaag tatcccaagg ctccggaagg |
| 1981 | gggcgaaggt gcccagccag tgcccgggca tgcccggaag acagcggttc aagacagaaa |
| 2041 | agatggcagc tcacctcctc tgttggagaa gcagaccgtt accaaagacg tcacagataa |
| 2101 | gccactagac ttgtcttcta aagtggtgga tgtagatgct tccaaagctg accacatgaa |
| 2161 | aaagatggct cccacggtcc tggttcacag cagggctgga agtggcttag tgctctccgg |
| 2221 | aagtgagatt ccgaaagaaa cactatctcc tccaggaaat ggttgtgcta tctatagatc |
| 2281 | tgaaatcatc agcactgctc cctcatcctg ggtggtgccc gggccaagtc ctaacgaaga |
| 2341 | gaacaatggc aaaagcatgt cgctgaaaaa caaggcattg gactgggcga taccacagca |
| 2401 | gcggagttca tcatgcccgc gcatgggcgg caccgatgct gtcatcacta acgtttcagg |
| 2461 | gtcagtgtcg agtgcaggcc gcccagcctc cgcatcaccc gcccccaatg ccaatgcaga |
| 2521 | tggcaccaaa accagcagga gctctgtaga aaccacacca tccgttattc agcacgtggg |
| 2581 | ccagcccccg gccactcctg ccaagcacag tagcagcacc agcagcaagg gcgccaaagc |
| 2641 | cagcaaccca gaaccgagtt tcaaagcaaa cgagaacggc cttccaccaa gctctatatt |
| 2701 | tctgtctcca aatgaggcat tcaggtcccc accaattccc taccccagga gttacctccc |
| 2761 | ttacccagcc cctgagggca ttgctgtaag tcccctctcc ttacatggca aaggacctgt |
| 2821 | ctaccctcac ccagttttgt tacccaatgg cagtctgttt cctgggcacc ttgccccaaa |
| 2881 | gcctgggctg ccctatgggc ttcccaccgg ccgtccagag tttgtgacct accaagatgc |
| 2941 | cctggggttg ggcatggtgc atcccatgtt gataccacac acgcccatag agattactaa |
| 3001 | agaggagaaa ccagagagga gatcccggtc ccatgagaga gcccgttacg aggacccaac |
| 3061 | cctccggaat cggttttccg agattttgga aactagcagc accaagttac atccagatgt |
| 3121 | ccccaccgac aagaacctaa agccgaaccc caactggaat caagggaaga ctgttgtcaa |
| 3181 | aagcgacaag cttgtctacg tagaccttct ccgagaagaa ccagatgcta aaactgacac |
| 3241 | aaacgtgtcc aaacccagct ttgcagcaga gagtgttggc cagagcgctg agccccccaa |
| 3301 | gccctcagtt gagccggccc tgcagcagca ccgtgatttc atcgccctga gagaggagtt |
| 3361 | ggggcgcatc agtgacttcc acgaaactta tactttcaaa cagccagtct tcaccgtaag |
| 3421 | caaggacagt gttctggcag gtaccaacaa agagaaccta gggttgccag tctcgactcc |
| 3481 | attcctggag ccacctctgg ggagcgatgg ccctgctgta acttttggta aaacccaaga |
| 3541 | ggatcccaaa ccattttgtg tgggcagtgc cccaccaagt gtggatgtga cccccaccta |
| 3601 | taccaaagat ggagctgatg aggctgaatc aaatgatggc aaagttctga aaccgaagcc |
| 3661 | atctaagctg gcaaagagaa tcgccaactc agcgggttac gtgggtgacc gattcaaatg |
| 3721 | tgtcactacc gaactgtatg cagattccag tcagctcagc cgggagcaac gggcattgca |
| 3781 | gatggaagga ttacaagagg acagtatttt atgtctaccc gctgcttact gtgagcgtgc |
| 3841 | aatgatgcgc ttctcagagt tggagatgaa agaaagagaa ggtggccacc cagcaaccaa |
| 3901 | agactccgag atgtgcaaat tcagcccagc cgactgggaa aggttgaaag gaaatcagga |
| 3961 | caaaaagcca aagtcggtca ccctggagga ggccattgca gaacagaacg aaagtgagag |
| 4021 | atgcgagtat agtgttggaa acaagcaccg tgatcccttt gaagccccag aggacaaaga |
| 4081 | tcttcctgtg gagaagtact ttgtggagag gcagcctgtg agcgagcctc ccgcagacca |
| 4141 | ggtggcctcg gacatgcctc acagccccac cctccgggtg gacaggaaac gcaaagtctc |
| 4201 | aggtgacagc agccacactg agaccactgc ggaggaggtg ccagaggacc ctctgctgaa |
| 4261 | agccaaacgc cgacgagtct ctaaagatga ctggcctgag agggaaatga caaacagttc |
| 4321 | ctctaaccac ttagaagacc cacattatag tgagctgacc aacctgaagg tgtgcattga |
| 4381 | attaacaggg ctccatccta aaaaacaacg ccacttgctg caccttagag aacgatggga |
| 4441 | gcagcaggtg tcggcagcag atggcaaacc tggccggcaa agcaggaagg aagtgaccca |
| 4501 | ggccactcag cctgaggcca ttcctcaggg gactaacatc actgaagaga aacctggcag |
| 4561 | gaaaagggca gaggccaaag gcaacagaag ctggtcggaa gagtctctta aacccagtga |
| 4621 | caatgaacaa ggcttgcctg tgttctccgg ctctccgccc atgaagagtc tttcatccac |
| 4681 | cagtgcaggc ggcaaaaagc aggctcagcc aagctgcgca ccagcctcca ggccgcctgc |
| 4741 | caaacagcag aaaattaaag aaaaccagaa gacagatgtg ctgtgtgcag acgaagaaga |
| 4801 | ggattgccag gctgcctccc tgctgcagaa atacaccgac aacagcgaga agccatccgg |
| 4861 | gaagagactg tgcaaaacca aacacttgat ccctcaggag tccaggcggg gattgccact |
| 4921 | gacaggggaa tactacgtgg agaatgccga tggcaaggtg actgtccgga gattcagaaa |
| 4981 | gcggccggag cccagttcgg actatgatct gtcaccagcc aagcaggagc caaagccctt |
| 5041 | cgaccgcttg cagcaactgc taccagcctc ccagtccaca cagctgccat gctcaagttc |
| 5101 | ccctcaggag accacccagt ctcgccctat gccgccggaa gcacggagac ttattgtcaa |
| 5161 | taagaacgct ggcgagaccc ttctgcagcg ggcagccagg cttggctatg aggaagtggt |
| 5221 | cctgtactgc ttagagaaca agatttgtga tgtaaatcat cgggacaacg caggttactg |
| 5281 | cgccctgcat gaagcttgtg ctaggggctg gctcaacatt gtgcgacacc tccttgaata |
| 5341 | tggcgctgat gtcaactgta gtgcccagga tggaaccagg cctctgcacg atgctgttga |
| 5401 | gaacgatcac ttggaaattg tccgactact tctctcttat ggtgctgacc ccaccttggc |
| 5461 | tacgtactca ggtagaacca tcatgaaaat gacccacagt gaacttatgg aaaagttctt |
| 5521 | aacagattat ttaaatgacc tccagggtcg caatgatgat gacgccagtg gcacttggga |
| 5581 | cttctatggc agctctgttt gtgaaccaga tgatgaaagt ggctatgatg ttttagccaa |
| 5641 | ccccccagga ccagaagacc aggatgatga tgacgatgcc tatagcgatg tgtttgaatt |
| 5701 | tgaattttca gagacccccc tcttaccgtg ttataacatc caagtatctg tggctcaggg |
| 5761 | gccacgaaac tggctactgc tttcggatgt ccttaagaaa ttgaaaatgt cctcccgcat |
| 5821 | atttcgctgc aattttccaa acgtggaaat tgtcaccatt gcagaggcag aattttatcg |
| 5881 | gcaggtttct gcaagtctct tgttctcttg ctccaaagac ctggaagcct tcaaccctga |
| 5941 | aagtaaggag ctgttagatc tggtggaatt cacgaacgaa attcagactc tgctgggctc |
| 6001 | ctctgtagag tggctccacc ccagtgatct ggcctcagac aactactggt gagcaagctg |
| 6061 | gacccaccat gtacagtgtg ttatagtgtt aatccttgtg catatgtgtc ataatacaac |
| 6121 | tatttctgta aagaaaggac actattacat atgaaaatat ctcttcttta tataagagaa |
| 6181 | attactccag tcagaaggac ttagaaacat gtttttttcc ttttaaactt ttaagtcagt |
| 6241 | ttttatgaag ttgttataat gtttctttac ttttcaatgc acacatgctt tgggatacgt |
| 6301 | ttgtttttac ttggaacatt tgtttctttt cttttttaag gagaaaaaaa aatgagtaaa |
| 6361 | aggagctcca cactttgact taatttcata caaagctctg atgacaggcc atgactgtag |
| 6421 | agtggtcaga actgtgtggt tggtttgagg gagcgaattc ggggaaggca cttggtgata |
| 6481 | taactttgtt ttgtttacag agtacctgct cgggccaggt aaatgctatt ggatgtaatc |
| 6541 | cagtagtgtg taatataaat tcaaaccata tccacacaca acaactaatt gtatgaaact |
| 6601 | tttatatcct aatttaaaag ctgtgaaatt agttttcacg catcaaaccg gattgtttat |
| 6661 | atgtttaaac attttatgct cttatttaaa gaagactttg agctattttt ttctgtaccc |
| 6721 | tgtaaaatat tgaaaactaa cataatatgt tgaggttgct tggaaatgta cataaaacta |
| 6781 | aaattttctg aatcgtgtgt ttatgtttga aatctgtgtt ttaactttgt aagtaaattc |
| 6841 | tctgcctttg tatttatatt ttacaaaaat tttcttaaaa ggcaataaaa ctgttgagga |
| 6901 | aaggagaaaa |

SEQ ID NO: 20 Human BCOR Isoform c (Encoded by Transcript Variant 5) Amino Acid
Sequence (NP_001116857.1)

| | |
|---|---|
| 1 | mlsatplygn vhswmnserv rmcgasedrk ilvndgdask arlelreenp lnhnvvdast |
| 61 | ahridglaal smdrtglire glrvpgnivy sslcglgsek greaatstig glgfssernp |
| 121 | emqfkpntpe tveasavsgk ppngfsaiyk tppgiqksav ataealgidr pasdkqspln |
| 181 | ingasylrlp wvnpymegat paiypfldsp nkyslnmyka llpqqsysla qplyspvctn |
| 241 | gerflylppp hyvgphipss laspmrlstp saspaipplv hcadkslpwk mgvspgnpvd |
| 301 | shayphiqns kqprvpsaka vtsglpgdta lllppsprps prvhlptqpa adtysefhkh |
| 361 | yaristspsv alskpymtvs sefpaarlsn gkypkapegg egaqpvpgha rktavqdrkd |
| 421 | gssppllekq tvtkdvtdkp idlsskvvdv daskadhmkk maptvivhsr agsglvlsgs |
| 481 | eipketlspp gngcaiyrse iistapsswv vpgpspneen ngksmslknk aldwaipqqr |
| 541 | ssscprmggt davitnvsgs vssagrpasa spapnanadg tktsrssvet tpsviqhvgq |
| 601 | ppatpakhss stsskgakas npepsfkane nglppssifl spneafrspp ipyprsylpy |
| 661 | papegiavsp lslhgkgpvy phpvllpngs ifpghlapkp glpyglptgr pefvtyqdal |
| 721 | glgmvhpmli phtpieitke ekperrsrsh eraryedptl rnrfseilet sstklhpdvp |
| 781 | tdknlkpnpn wnqgktvvks dklvyvdllr eepdaktdtn vskpsfaaes vgqsaeppkp |
| 841 | svepalqqhr dfialreelg risdfhetyt fkqpvftvsk dsvlagtnke nlglpvstpf |
| 901 | lepplgsdgp avtfgktqed pkpfcvgsap psvdvtptyt kdgadeaesn dgkvlkpkps |
| 961 | klakriansa gyvgdrfkcv ttelyadssq lsreqralqm eglqedsilc lpaayceram |
| 1021 | mrfselemke regghpatkd semckfspad werlkgnqdk kpksvtleea iaeqneserc |
| 1081 | eysvgnkhrd pfeapedkdl pvekyfverq pvseppadqv asdmphspti rvdrkrkvsg |
| 1141 | dsshtettae evpedpllka krrrvskddw peremtnsss nhledphyse ltnlkvciel |
| 1201 | tglhpkkqrh llhlrerweq qvsaadgkpg rqsrkevtqa tqpeaipqgt niteekpgrk |
| 1261 | raeakgnrsw seeslkpsdn eqglpvfsgs ppmkslssts aggkkqaqps capasrppak |
| 1321 | qqkikenqkt dvicadeeed cqaasllqky tdnsekpsgk ricktkhlip qesrrglplt |
| 1381 | geyyvenadg kvtvrrfrkr pepssdydls pakqepkpfd rlqqllpasq stqlpcsssp |
| 1441 | qettqsrpmp pearrlivnk nagetllqra arlgyeevvl yclenkicdv nhrdnagyca |
| 1501 | iheacargwl nivrhileyg advncsaqdg trplhdaven dhleivrlll sygadptlat |
| 1561 | ysgrtimkmt hselmekfit dylndlqgrn dddasgtwdf ygssvcepdd esgydvlanp |
| 1621 | pgpedqdddd daysdvfefe fsetpllpcy niqvsvaqgp rnwlllsdvl kklkmssrif |
| 1681 | rcnfpnveiv tiaeaefyrq vsasllfscs kdleafnpes kelldiveft neiqtllgss |
| 1741 | vewlhpsdla sdnyw |

SEQ ID NO: 21 Human YY1 associated factor 2 (YAF2) Transcript Variant 2 cDNA
Sequence (NM_005748.6; CDS region from 69-611)

| | |
|---|---|
| 1 | attatcctcc ttattgacaa acagagcggt cgcggcggcg actctcggcg tgcggtgata |
| 61 | gccaagccat gggagacaag aagagcccca ccaggccgaa gcggcagccg aagccgtcct |
| 121 | cggatgaggg ttactgggac tgtagcgtct gcaccttccg gaacagcgcc gaggccttca |
| 181 | agtgcatgat gtgcgatgtg cggaagggca cctccacccg gaaacctcga cctgtctccc |
| 241 | agttggttgc acagcaggtt actcagcagt ttgtgcctcc tacacagtca aagaaagaga |
| 301 | aaaaagataa agtagaaaaa gaaaaaagtg aaaaggaaac aactagcaaa aagaatagcc |
| 361 | ataagaaaac caggccaaga ttgaaaaatg tggatcggag tagtgctcag catttggaag |
| 421 | ttactgttgg agatctgaca gtcattatta cagactttaa ggagaaaaca aagtcaccgc |
| 481 | ctgcatctag tgctgcttct gcagatcaac acagtcaaag cggctctagc tctgataaca |
| 541 | cagagagagg aatgtccagg tcatcttcac ccagaggaga agcctcatca ttgaatggag |
| 601 | aatctcatta aagtttattt tctccaattt cttagtcact tctgtcctac catgcaaata |
| 661 | cacagattat gccaagaggt accacatttt catgacagat acattcatgc acaatccata |
| 721 | atttgagttt tacataaaat agaaatttgt tagaatttgt tagattttat tgcaatgatg |
| 781 | cctaccaaac atttccagac ttaacatttt ggtctctgca gttaagtgcc atgaaaatgt |
| 841 | ggttgaatta ttcattatgc agtgttattg gtaagtgtat tttcactttt agtttagtga |
| 901 | attctaacac ataattcttg aattctctac tattggcatg taacgaattt aaatttttta |
| 961 | taacatagtg caagctgcct aaatatgtat tatttgagaa ttgtgaaaca gatagttata |
| 1021 | tgtatacaag tcaaagaaca acttaactat tgctgcaaca ggtttttctt aatggttatc |
| 1081 | ctcttaaata cacctgctgg tacttggtgt ggttaaatag gaaaattgtt attaaataaa |
| 1141 | gaatttgtat gaacctttgc caatgttttt gacacgtttt actaattatt gttcctgaat |
| 1201 | tatgtttctg gttttatcta ttttttgagg tttttttgtt tgcttatttt tcaaaacatt |
| 1261 | catttattgt aatgtttact agcggactag taaacaataa aacattgatt atttagcttt |
| 1321 | ataattcagg tttagtgcta ttgtcattga acactggtat tttctgtatc atataaaaca |
| 1381 | ttaaaattca aataattata agcatttggc aaaaacaaga gaaaagaaac ttgccatatt |
| 1441 | ttacaagctg caattttaga aaagctttaa cttaatgata gttttatcat tgttttcttg |
| 1501 | tcccaaactt atccagggcc atagaagtat gaatctaatt aaaacagaaa tgggaattat |
| 1561 | tgcacagaaa tgggaaataa ctaattttaa atcagtcaaa ttggcttctt attaaataca |
| 1621 | ataattctta tgaaaatcat agtaccctat tttcagacac agctgccagt ttacacattt |
| 1681 | ctcagtatcc tgaaaggaaa aaagtatagc cccacttata ctatgtaaaa ttaccaataa |
| 1741 | aatattttta tgactacaga ttttgcattt ttgtttacaa ctatttaaag agttttatgt |
| 1801 | tgtatttaga atttcaacct agaaaccaca cagtacttaa attctcctgg ggtctcctgc |
| 1861 | tttctcttaa ccatttgctt aatatatatc tacctaaagg agacttctga attgtaaatg |
| 1921 | aacttaaaaa tagaatgtgg atgcaaaata tcacataaga catcatgata acatttgaag |
| 1981 | aaaaaataaa actgtagacc ctaacagttg tgatatttgg tggtttcatg tggtaatgta |
| 2041 | attttctgtt taattacagt actttttaca ggcacagtgg tactgtcttt tttgtaagat |
| 2101 | gctagttgtg aaatacaatt aattgcatac agtaaaagtc tgtgattaaa acatttatat |
| 2161 | acctcattct ttagtgttgt taaatgaaaa attaaaattt gtgtttatta taagatactt |
| 2221 | tcaatggaat accagctaac cagatatgtt cctttaaaac atgaattttt ttgtcttatt |
| 2281 | ctgtttttaa catgtttcaa atgttttata catttttgga gatagtaaaa atttaaaaat |
| 2341 | gtaataggga aaatatttaa tttttaagtc aaaaactatg ctttaaagga attacatctt |
| 2401 | atgatcaaat aactttcagg atgtatttta aatgagctgc aagagagaaa aatctgacag |
| 2461 | gagatgggat tttacctgaa tggagcatac tcatatttct acataggtgg gaagatactc |
| 2521 | atctttctac atagacggga acacggtgta gcatggagat taagcgtgct aactgacacc |
| 2581 | tcatgtttga atcctgttgt agaatggaaa tgagcttaaa ttacccaagc ttactttcca |
| 2641 | tctataaaac agggctaata atggtaatca catctaattt atagggtttt tgggaggatt |
| 2701 | aagtcaatcc gtgtaaaatt cttaattatc tgacacatac tggtcactca gtaaatgtga |
| 2761 | gcttttacca ttgttatagg taaaatccca ttacaaaaat acaaaaatat tttttgtaac |
| 2821 | agttattttc ctgccctctt ggatgaccta aagtaatagt gttttataga tttcactaaa |
| 2881 | ttttcagtgt aatatcagat gttttctctg gatgcagaaa gaccatcttt ccataattaa |
| 2941 | agaacctggt gggtgtgcac tatgagattg gagaaatgaa ttaagttgac ttgaaattat |
| 3001 | tttactttta ttaatcacag gtatctcaac ctgcctttgt ttcaccctac ttcaagtaca |
| 3061 | cttccacacc aggaaaacag acccaaattc cataaacaga actgacgtta aaatatgcga |
| 3121 | aagattcaga acctaggttt agggtacatg tttttctctg tttcatgaac ttacagtgtg |
| 3181 | gtttgagcac tgggttcttt tagccaaatt ccatacaaaa agaatttgga tgttttccag |
| 3241 | catttattac cttactttgc tataagtaag agtgaagata ggccgggtgt gatggctcat |
| 3301 | gccagtaatc ccagcacttt gggctgaagc aggcagatat tttgagccca ggaccagcct |
| 3361 | gggcaacatg gtgaaagctc atctctatga aaaaatttga atatacatat atatatatat |
| 3421 | gtatatttaa aaagtgaata taatttgagg cacagaagaa tattcttaaa accttttgta |
| 3481 | ttatacaata gaaataaagg ttttattttt attaaatgct ttcctaaaat gatagtggat |
| 3541 | aatagacaaa gtgaaaacat ttaaaaagga agagaaactt cagcctttct aagttgatag |
| 3601 | catgtttatt aacttttaga taaagatcct tatagactga aagaatgtag cctctgcatt |
| 3661 | aatggtaaat ggtctagaag ttttcgtttc cactgaggct tccgccactc gcttttttaa |
| 3721 | acttcctgct atggtttgga tatggtttgt cctcaccaaa actcatgctg aggcctgagt |
| 3781 | ccccagtttg attgtgttgg taggtggtgc ctttaagagg tgattaggtc attgagatgg |
| 3841 | attaaaggct ttctcatgag gctcagttag ttctggaatg gattcgctct tgcaggaatg |
| 3901 | gatgaattct cacaagagtg ggttgttatg aagtgaggat gcttcttgtg ttctgttctc |
| 3961 | tttgccctcc tcagttcacc atctgctttc caccatgagt tgaagcagca tgaggccctc |
| 4021 | atcagatggg ctccctgatc ttggactcct cagcctttgg actcataagc caaaataaat |
| 4081 | ctgttttctt tataaa |

SEQ ID NO: 22 Human YY1-associated factor 2 isoform 2 (Encoded by Transcript
Variant 2) Amino Acid Sequence (NP 005739.2)

| | |
|---|---|
| 1 | mgdkksptrp krqpkpssde gywdcsvctf rnsaeafkcm mcdvrkgtst rkprpvsqlv |
| 61 | aqqvtqqfvp ptqskkekkd kvekekseke ttskknshkk trprlknvdr ssaqhlevtv |
| 121 | gdltviitdf kektksppas saasadqhsq sgsssdnter gmsrsssprg easslngesh |

*Included in Table 5, as well as in Tables 1-4 described herein, are RNA nucleic acid molecules (e.g., thymines replaced with uridines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 5, as well as in Tables 1-4 described herein, or a portion thereof. Such nucleic acid molecules can have a function of the full-length nucleic acid as described further herein.
*Included in Table 5, as well as in Tables 1-4 described herein, are orthologs of the proteins, such as in human, mouse, monkey, etc., as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 5, as well as in Tables 1-4 described herein, or a portion thereof. Such polypeptides can have a function of the full-length polypeptide as described further herein.

### II. Subjects

In one embodiment, the subject for whom predicted likelihood of efficacy of an inhibitor of one or more biomarkers listed in Tables 1-5, and an immunotherapy combination treatment is determined, is a mammal (e.g., mouse, rat, primate, non-human mammal, domestic animal, such as a dog, cat, cow, horse, and the like), and is preferably a human. In another embodiment, the subject is an animal model of cancer. For example, the animal model can be an orthotopic xenograft animal model of a human-derived cancer.

In another embodiment of the methods of the present invention, the subject has not undergone treatment, such as chemotherapy, radiation therapy, targeted therapy, and/or immunotherapies. In still another embodiment, the subject has undergone treatment, such as chemotherapy, radiation therapy, targeted therapy, and/or immunotherapies.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient. The methods of the present invention can be used to determine the responsiveness to inhibitor(s) of one or more biomarkers listed in Tables 1-5, and immunotherapy combination treatment of many different cancers in subjects such as those described herein.

### III. Sample Collection, Preparation and Separation

In some embodiments, biomarker amount and/or activity measurement(s) in a sample derived from a subject is compared to a predetermined control (standard) sample. The sample from the subject is typically from a diseased tissue, such as cancer cells or tissues. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects.

In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) may be a biomarker amount and/or activity measurement(s) used to, by way of example only, evaluate a subject that may be selected for treatment (e.g., based on the number of genomic mutations and/or the number of genomic mutations causing non-functional proteins for DNA repair genes), evaluate a response to an inhibitor of one or more biomarkers listed in Tables 1-5, and an immunotherapy combination treatment, and/or evaluate a response to an inhibitor of one or more biomarkers listed in Tables 1-5, and an immunotherapy combination treatment with one or more additional anti-cancer therapies. A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually.

In one embodiment, the amounts determined and/or compared in a method described herein are based on absolute measurements. In another embodiment, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of anti-cancer therapy. Post-treatment biomarker measurement can be made at any time after initiation of anticancer therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of anti-cancer therapy, and even longer toward indefinitely for continued monitoring. Treatment can comprise anti-cancer therapy, such as a therapeutic regimen comprising one or more inhibitors of one or more biomarkers listed in Tables 1-5, and immunotherapy combination treatment alone or in combination with other anti-cancer agents, such as with immune checkpoint inhibitors.

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change of biomarker amount and/or activity measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to posttreatment biomarker measurement. Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. In another embodiment, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids. The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermeable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermeable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray. Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

### IV. Biomarker Nucleic Acids and Polypeptides

One aspect of the present invention pertains to the use of isolated nucleic acid molecules that correspond to biomarker nucleic acids that encode a biomarker polypeptide or a portion of such a polypeptide. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences that naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A biomarker nucleic acid molecule of the present invention can be isolated using standard molecular biology techniques and the sequence information in the database records described herein. Using all or a portion of such nucleic acid sequences, nucleic acid molecules of the present invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the present invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the present invention can be prepared by standard synthetic techniques, e.g., using an automated DNA synthesizer.

Moreover, a nucleic acid molecule of the present invention can comprise only a portion of a nucleic acid sequence, wherein the full length nucleic acid sequence comprises a marker of the present invention or which encodes a polypeptide corresponding to a marker of the present invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a biomarker nucleic acid sequence. Probes based on the sequence of a biomarker nucleic acid molecule can be used to detect transcripts or genomic sequences corresponding to one or more markers of the present invention. The probe comprises a label group attached thereto, e.g., a radioisotope, a fluorescent compound, an enzyme, or an enzyme co-factor.

A biomarker nucleic acid molecules that differ, due to degeneracy of the genetic code, from the nucleotide sequence of nucleic acid molecules encoding a protein which corresponds to the biomarker, and thus encode the same protein, are also contemplated.

In addition, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. For example, biomarker alleles can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect on the function of the protein.

As used herein, the terms “gene” and “recombinant gene” refer to nucleic acid molecules comprising an open reading frame encoding a polypeptide corresponding to a marker of the present invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the present invention.

In another embodiment, a biomarker nucleic acid molecule is at least 7, 15, 20, 25, 30, 40, 60, 80, 100, 150, 200, 250, 300, 350, 400, 450, 550, 650, 700, 800, 900, 1000, 1100, 1200, 1300, 1400, 1500, 1600, 1700, 1800, 1900, 2000, 2200, 2400, 2600, 2800, 3000, 3500, 4000, 4500, or more nucleotides in length and hybridizes under stringent conditions to a nucleic acid molecule corresponding to a marker of the present invention or to a nucleic acid molecule encoding a protein corresponding to a marker of the present invention. As used herein, the term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule of the present invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “nonessential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the present invention pertains to nucleic acid molecules encoding a polypeptide of the present invention that contain changes in amino acid residues that are not essential for activity. Such polypeptides differ in amino acid sequence from the naturally-occurring proteins which correspond to the markers of the present invention, yet retain biological activity. In one embodiment, a biomarker protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of a biomarker protein described herein.

An isolated nucleic acid molecule encoding a variant protein can be created by introducing one or more nucleotide substitutions, additions or deletions into the nucleotide sequence of nucleic acids of the present invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In some embodiments, the present invention further contemplates the use of anti-biomarker antisense nucleic acid molecules, i.e., molecules which are complementary to a sense nucleic acid of the present invention, e.g., complementary to the coding strand of a double-stranded cDNA molecule corresponding to a marker of the present invention or complementary to an mRNA sequence corresponding to a marker of the present invention. Accordingly, an antisense nucleic acid molecule of the present invention can hydrogen bond to (i.e. anneal with) a sense nucleic acid of the present invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the present invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the present invention are typically administered to a subject or generated in situ such that they hybridize with or bind to cellular mRNA and/or genomic DNA encoding a polypeptide corresponding to a selected marker of the present invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the present invention includes direct injection at a tissue site or infusion of the antisense nucleic acid into a blood- or bone marrow-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the present invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The present invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the present invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the present invention can be used to select a catalytic RNA having a specific ribonuclease activity from a pool of RNA molecules (see, e.g., Bartel and Szostak, 1993, Science 261:1411-1418).

The present invention also encompasses nucleic acid molecules which form triple helical structures. For example, expression of a biomarker protein can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. U.S.A. 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. U.S.A. 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. U.S.A. 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

Another aspect of the present invention pertains to the use of biomarker proteins and biologically active portions thereof. In one embodiment, the native polypeptide corresponding to a marker can be isolated from cells or tissue sources by an appropriate purification scheme using standard protein purification techniques. In another embodiment, polypeptides corresponding to a marker of the present invention are produced by recombinant DNA techniques. Alternative to recombinant expression, a polypeptide corresponding to a marker of the present invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly, such preparations of the protein have less than about 30%, 20%, 10%, 5% (by dry weight) of chemical precursors or compounds other than the polypeptide of interest.

Biologically active portions of a biomarker polypeptide include polypeptides comprising amino acid sequences sufficiently identical to or derived from a biomarker protein amino acid sequence described herein, but which includes fewer amino acids than the full length protein, and exhibit at least one activity of the corresponding full-length protein. Typically, biologically active portions comprise a domain or motif with at least one activity of the corresponding protein. A biologically active portion of a protein of the present invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the present invention.

Preferred polypeptides have an amino acid sequence of a biomarker protein encoded by a nucleic acid molecule described herein. Other useful proteins are substantially identical (e.g., at least about 40%, preferably 50%, 60%, 70%, 75%, 80%, 83%, 85%, 88%, 90%, 91%, 92%, 93%, 94%, 95%, 96%, 97%, 98%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. U.S.A. 87:2264-2268, modified as in Karlin and Altschul (1993) Proc. Natl. Acad. Sci. U.S.A. 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the present invention. BLAST protein searches can be performed with the XBLAST program, score=50, wordlength=3 to obtain amino acid sequences homologous to a protein molecules of the present invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See the World Wide Web at ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. U.S.A. 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted. The present invention also provides chimeric or fusion proteins corresponding to a biomarker protein. As used herein, a “chimeric protein” or “fusion protein” comprises all or part (preferably a biologically active part) of a polypeptide corresponding to a marker of the present invention operably linked to a heterologous polypeptide (i.e., a polypeptide other than the polypeptide corresponding to the marker). Within the fusion protein, the term “operably linked” is intended to indicate that the polypeptide of the present invention and the heterologous polypeptide are fused in-frame to each other. The heterologous polypeptide can be fused to the amino-terminus or the carboxyl-terminus of the polypeptide of the present invention.

One useful fusion protein is a GST fusion protein in which a polypeptide corresponding to a marker of the present invention is fused to the carboxyl terminus of GST sequences. Such fusion proteins can facilitate the purification of a recombinant polypeptide of the present invention.

In another embodiment, the fusion protein contains a heterologous signal sequence, immunoglobulin fusion protein, toxin, or other useful protein sequence. Chimeric and fusion proteins of the present invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the present invention can be cloned into such an expression vector such that the fusion moiety is linked in-frame to the polypeptide encompassed by the present invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the present invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the biomarker polypeptides described herein. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a biomarker protein that function as either agonists (mimetics) or as antagonists can be identified by screening combinatorial libraries of mutants, e.g., truncation mutants, of the protein of the present invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the present invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the present invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the present invention (Arkin and Yourvan, 1992, Proc. Natl. Acad. Sci. U.S.A. 89:7811-7815; Delgrave et al., 1993, Protein Engineering 6(3):327-331). An isolated polypeptide or a fragment thereof (or a nucleic acid encoding such a polypeptide) corresponding to one or more biomarkers encompassed by the present invention, including the biomarkers listed in Tables 1-5 or fragments thereof, can be used as an immunogen to generate antibodies that bind to said immunogen, using standard techniques for polyclonal and monoclonal antibody preparation according to well-known methods in the art. An antigenic peptide comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein).

In some embodiments, the immunotherapy utilizes an inhibitor of at least one immune checkpoint, such as an antibody binds substantially specifically to an immune checkpoint, such as PD-1, and inhibits or blocks its immunoinhibitory function, such as by interrupting its interaction with a binding partner of the immune checkpoint, such as PD-L1 and/or PD-L2 binding partners of PD-1. In one embodiment, an antibody, especially an intrabody, binds substantially specifically to one or more biomarkers listed in Tables 1-5, and inhibits or blocks its biological function. In another embodiment, an antibody, especially an intrabody, binds substantially specifically to the binding partner(s) of one or more biomarkers listed in Tables 1-5, such as substrates of such one or more biomarkers described herein, and inhibits or blocks its biological function, such as by interrupting its interaction to one or more biomarkers listed in Tables 1-5.

For example, a polypeptide immunogen typically is used to prepare antibodies by immunizing a suitable subject (e.g., rabbit, goat, mouse or other mammal) with the immunogen. A preferred animal is a mouse deficient in the desired target antigen. For example, a PD-1 knockout mouse if the desired antibody is an anti-PD-1 antibody, may be used. This results in a wider spectrum of antibody recognition possibilities as antibodies reactive to common mouse and human epitopes are not removed by tolerance mechanisms. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique (originally described by Kohler and Milstein (1975) Nature 256:495-497) (see also Brown et al. (1981) J Immunol. 127:539-46; Brown et al. (1980) J. Biol. Chem. 255:4980-83; Yeh et al. (1976) Proc. Natl. Acad. Sci. 76:2927-31; Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well-known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically. In some embodiments, the immunization is performed in a cell or animal host that has a knockout of a target antigen of interest (e.g., does not produce the antigen prior to immunization).

Any of the many well-known protocols used for fusing lymphocytes and immortalized cell lines can be applied for the purpose of generating a monoclonal antibody against one or more biomarkers encompassed by the present invention, including the biomarkers listed in Tables 1-5, or a fragment thereof (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful.

Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O—Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody encompassed by the present invention are detected by screening the hybridoma culture supernatants for antibodies that bind a given polypeptide, e.g., using a standard ELISA assay.

As an alternative to preparing monoclonal antibody-secreting hybridomas, a monoclonal specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. U.S.A. 89:3576-3580; Garrard et al. (1991) (NY) 9:1373-1377; Hoogenboom et al. (1991)Nucleic Acids Res. 19:4133-4137; Barbas et al. (1991) Proc. Natl. Acad. Sci. U.S.A. 88:7978-7982; and McCafferty et al. (1990) Nature 348:552-554.

Since it is well-known in the art that antibody heavy and light chain CDR3 domains play a particularly important role in the binding specificity/affinity of an antibody for an antigen, the recombinant monoclonal antibodies of the present invention prepared as set forth above preferably comprise the heavy and light chain CDR3s of variable regions of the antibodies described herein and well-known in the art. Similarly, the antibodies can further comprise the CDR2s of variable regions of said antibodies. The antibodies can further comprise the CDR1s of variable regions of said antibodies. In other embodiments, the antibodies can comprise any combinations of the CDRs.

The CDR1, 2, and/or 3 regions of the engineered antibodies described above can comprise the exact amino acid sequence(s) as those of variable regions of the present invention described herein. However, the ordinarily skilled artisan will appreciate that some deviation from the exact CDR sequences may be possible while still retaining the ability of the antibody, especially an intrabody, to bind a desired target, such as one or more biomarkers listed in Tables 1-5, and/or a binding partner thereof, either alone or in combination with an immunotherapy, such as the one or more biomarkers, the binding partners/substrates of such biomarkers, or an immunotherapy effectively (e.g., conservative sequence modifications). Accordingly, in another embodiment, the engineered antibody may be composed of one or more CDRs that are, for example, 50%, 60%, 70%, 80%, 85%, 90%, 91%, 92%, 93%, 94%, 95%, 96%, 97%, 98%, 99%, or 99.5% identical to one or more CDRs of the present invention described herein or otherwise publicly available.

For example, the structural features of non-human or human antibodies (e.g., a rat anti-mouse/anti-human antibody) can be used to create structurally related human antibodies, especially intrabodies, that retain at least one functional property of the antibodies of the present invention, such as binding to one or more biomarkers listed in Tables 1-5, the binding partners/substrates of such one or more biomarkers, and/or an immune checkpoint. Another functional property includes inhibiting binding of the original known, non-human or human antibodies in a competition ELISA assay.

Antibodies, immunoglobulins, and polypeptides encompassed by the present invention can be used in an isolated (e.g., purified) form or contained in a vector, such as a membrane or lipid vesicle (e.g. a liposome). Moreover, amino acid sequence modification(s) of the antibodies described herein are contemplated. For example, it may be desirable to improve the binding affinity and/or other biological properties of the antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and can be corrected by replacing the amino acids with amino acid residues of the original antibody derived from a non-human animal.

Similarly, modifications and changes may be made in the structure of the antibodies described herein, and in the DNA sequences encoding them, and still obtain a functional molecule that encodes an antibody and polypeptide with desirable characteristics. For example, antibody glycosylation patterns can be modulated to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr et al. (1987) and by Edge et al. (1981). Enzymatic cleavage of carbohydrate moieties on antibodies can be achieved by the use of a variety of endo- and exo-glycosidases as described by Thotakura et al. (1987).

Other modifications can involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Conjugation of antibodies or other proteins of the present invention with heterologous agents can be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

In another aspect, the present invention features antibodies conjugated to a therapeutic moiety, such as a cytotoxin, a drug, and/or a radioisotope. When conjugated to a cytotoxin, these antibody conjugates are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). An antibody of the present invention can be conjugated to a radioisotope, e.g., radioactive iodine, to generate cytotoxic radiopharmaceuticals for treating a related disorder, such as a cancer.

Conjugated antibodies, in addition to therapeutic utility, can be useful for diagnostically or prognostically to monitor polypeptide levels in tissue as part of a clinical testing procedure, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include a flag tag, a myc tag, an hemagglutinin (HA) tag, streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include ¹²⁵I, ¹³¹I, ³⁵S, or ³H. As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or indocyanine (Cy5)) to the antibody, as well as indirect labeling of the antibody by reactivity with a detectable substance.

The antibody conjugates of the present invention can be used to modify a given biological response. The therapeutic moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, Pseudomonas exotoxin, or diphtheria toxin; a protein such as tumor necrosis factor or interferon-.gamma.; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other cytokines or growth factors.

In one embodiment, an antibody for use in the instant invention is a bispecific or multispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. U.S.A., 83:1453, and Staerz and Bevan (1986) Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a polypeptide or a fragment thereof of one or more biomarkers encompassed by the present invention, including one or more biomarkers listed in Tables 1-5, or a fragment thereof. In one embodiment, the bispecific antibody could specifically bind to both a polypeptide or a fragment thereof and its natural binding partner(s) or a fragment(s) thereof. Techniques for modulating antibodies, such as humanization, conjugation, recombinant techniques, and the like are well-known in the art.

In another aspect of this invention, peptides or peptide mimetics can be used to modulate expression (e.g., increase expression or decrease expression) and/or activity (e.g., agonize or antagonize) of one or more biomarkers encompassed by the present invention, including one or more biomarkers listed in Tables 1-5, or a fragment(s) thereof. In one embodiment, variants of one or more biomarkers listed in Tables 1-5 that function as a modulating agent for the respective full length protein, can be identified by screening combinatorial libraries of mutants, e.g., truncation mutants, for antagonist activity. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad. Sci. U.S.A. 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes one or more biomarkers encompassed by the present invention, including one or more biomarkers listed in Tables 1-5, or a fragment thereof. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide. The amino acid sequences described herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well-known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments encompassed by the present invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides described herein can be used therapeutically to treat disease, e.g., by altering costimulation in a patient.

Peptidomimetics (Fauchere (1986) Adv. Drug Res. 15:29; Veber and Freidinger (1985) TINS p.392; and Evans et al. (1987) J Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH2S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivatization (e.g., labeling) of peptidomimetics should not substantially interfere with the desired biological or pharmacological activity of the peptidomimetic.

Also encompassed by the present invention are small molecules which can modulate (either enhance or inhibit) interactions, e.g., between biomarkers described herein or listed in Tables 1-5 and their natural binding partners. The small molecules of the present invention can be obtained using any of the numerous approaches in combinatorial library methods known in the art, including: spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145).

Examples of methods for the synthesis of molecular libraries can be found in the art, for example in: DeWitt et al. (1993) Proc. Natl. Acad Sci. U.S.A. 90:6909; Erb et al. (1994) Proc. Natl. Acad Sci. U.S.A. 91:11422; Zuckermann et al. (1994) J. Med Chem. 37:2678; Cho et al. (1993) Science 261:1303; Carrell et al. (1994) Angew. Chem. Int. Ed Engl. 33:2059; Carell et al. (1994) Angew. Chem. Int. Ed Engl. 33:2061; and in Gallop et al. (1994) J. Med Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad Sci. U.S.A. 89:1865-1869) or on phage (Scott and Smith (1990) Science 249:386-390); (Devlin (1990) Science 249:404-406); (Cwirla et al. (1990) Proc. Natl. Acad Sci. U.S.A. 87:6378-6382); (Felici (1991) J. Mol. Biol. 222:301-310); (Ladner supra.). Compounds can be screened in cell based or non-cell based assays. Compounds can be screened in pools (e.g. multiple compounds in each testing sample) or as individual compounds.

Chimeric or fusion proteins can be prepared for the inhibitor(s) of one or more biomarkers listed in Tables 1-5, and/or agents for the immunotherapies described herein, such as inhibitors to the biomarkers encompassed by the present invention, including the biomarkers listed in Tables 1-5, or fragments thereof. As used herein, a “chimeric protein” or “fusion protein” comprises one or more biomarkers encompassed by the present invention, including one or more biomarkers listed in Tables 1-5, or a fragment thereof, operatively linked to another polypeptide having an amino acid sequence corresponding to a protein which is not substantially homologous to the respective biomarker. In a preferred embodiment, the fusion protein comprises at least one biologically active portion of one or more biomarkers encompassed by the present invention, including one or more biomarkers listed in Tables 1-5, or fragments thereof. Within the fusion protein, the term “operatively linked” is intended to indicate that the biomarker sequences and the non-biomarker sequences are fused in-frame to each other in such a way as to preserve functions exhibited when expressed independently of the fusion. The “another” sequences can be fused to the N-terminus or C-terminus of the biomarker sequences, respectively.

Such a fusion protein can be produced by recombinant expression of a nucleotide sequence encoding the first peptide and a nucleotide sequence encoding the second peptide. The second peptide may optionally correspond to a moiety that alters the solubility, affinity, stability or valency of the first peptide, for example, an immunoglobulin constant region. In another preferred embodiment, the first peptide consists of a portion of a biologically active molecule (e.g. the extracellular portion of the polypeptide or the ligand binding portion). The second peptide can include an immunoglobulin constant region, for example, a human Cγ1 domain or Cγ 4 domain (e.g., the hinge, CH2 and CH3 regions of human IgCγ1, or human IgCγ4, see e.g., Capon et al. U.S. Pat. Nos. 5,116,964; 5,580,756; 5,844,095 and the like, incorporated herein by reference). Such constant regions may retain regions which mediate effector function (e.g. Fc receptor binding) or may be altered to reduce effector function. A resulting fusion protein may have altered solubility, binding affinity, stability and/or valency (i.e., the number of binding sites available per polypeptide) as compared to the independently expressed first peptide, and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well-known in the art. Protein and peptides can be isolated from cell culture media, host cells, or both using techniques known in the art for purifying proteins and peptides. Techniques for transfecting host cells and purifying proteins and peptides are known in the art.

Preferably, a fusion protein encompassed by the present invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992).

The fusion proteins encompassed by the present invention can be used as immunogens to produce antibodies in a subject. Such antibodies may be used to purify the respective natural polypeptides from which the fusion proteins were generated, or in screening assays to identify polypeptides which inhibit the interactions between one or more biomarkers polypeptide or a fragment thereof and its natural binding partner(s) or a fragment(s) thereof.

Also provided herein are compositions comprising one or more nucleic acids comprising or capable of expressing at least 1, 2, 3, 4, 5, 10, 20 or more small nucleic acids or antisense oligonucleotides or derivatives thereof, wherein said small nucleic acids or antisense oligonucleotides or derivatives thereof in a cell specifically hybridize (e.g., bind) under cellular conditions, with cellular nucleic acids (e.g., small non-coding RNAS such as miRNAs, pre-miRNAs, pri-miRNAs, miRNA*, anti-miRNA, a miRNA binding site, a variant and/or functional variant thereof, cellular mRNAs or a fragments thereof). In one embodiment, expression of the small nucleic acids or antisense oligonucleotides or derivatives thereof in a cell can inhibit expression or biological activity of cellular nucleic acids and/or proteins, e.g., by inhibiting transcription, translation and/or small nucleic acid processing of, for example, one or more biomarkers encompassed by the present invention, including one or more biomarkers listed in Tables 1-5, or fragment(s) thereof. In one embodiment, the small nucleic acids or antisense oligonucleotides or derivatives thereof are small RNAs (e.g., microRNAs) or complements of small RNAs. In another embodiment, the small nucleic acids or antisense oligonucleotides or derivatives thereof can be single or double stranded and are at least six nucleotides in length and are less than about 1000, 900, 800, 700, 600, 500, 400, 300, 200, 100, 50, 40, 30, 25, 24, 23, 22, 21, 20, 19, 18, 17, 16, 15, or 10 nucleotides in length. In another embodiment, a composition may comprise a library of nucleic acids comprising or capable of expressing small nucleic acids or antisense oligonucleotides or derivatives thereof, or pools of said small nucleic acids or antisense oligonucleotides or derivatives thereof. A pool of nucleic acids may comprise about 25, 5-10, 10-20, 10-30 or more nucleic acids comprising or capable of expressing small nucleic acids or antisense oligonucleotides or derivatives thereof. In one embodiment, binding may be by conventional base pair complementarity, or, for example, in the case of binding to DNA duplexes, through specific interactions in the major groove of the double helix. In general, “antisense” refers to the range of techniques generally employed in the art, and includes any process that relies on specific binding to oligonucleotide sequences.

It is well-known in the art that modifications can be made to the sequence of a miRNA or a pre-miRNA without disrupting miRNA activity. As used herein, the term “functional variant” of a miRNA sequence refers to an oligonucleotide sequence that varies from the natural miRNA sequence, but retains one or more functional characteristics of the miRNA (e.g. cancer cell proliferation inhibition, induction of cancer cell apoptosis, enhancement of cancer cell susceptibility to chemotherapeutic agents, specific miRNA target inhibition). In some embodiments, a functional variant of a miRNA sequence retains all of the functional characteristics of the miRNA. In certain embodiments, a functional variant of a miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or precursor thereof over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain embodiments the nucleobase sequence of a functional variant is capable of hybridizing to one or more target sequences of the miRNA.

miRNAs and their corresponding stem-loop sequences described herein may be found in miRBase, an online searchable database of miRNA sequences and annotation, found on the world wide web at microrna.sanger.ac.uk. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence.

In some embodiments, miRNA sequences encompassed by the present invention may be associated with a second RNA sequence that may be located on the same RNA molecule or on a separate RNA molecule as the miRNA sequence. In such cases, the miRNA sequence may be referred to as the active strand, while the second RNA sequence, which is at least partially complementary to the miRNA sequence, may be referred to as the complementary strand. The active and complementary strands are hybridized to create a double-stranded RNA that is similar to a naturally occurring miRNA precursor. The activity of a miRNA may be optimized by maximizing uptake of the active strand and minimizing uptake of the complementary strand by the miRNA protein complex that regulates gene translation. This can be done through modification and/or design of the complementary strand.

In some embodiments, the complementary strand is modified so that a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules known in the art, including NH₂, NHCOCH₃, and biotin.

In another embodiment, the uptake of the complementary strand by the miRNA pathway is reduced by incorporating nucleotides with sugar modifications in the first 2-6 nucleotides of the complementary strand. It should be noted that such sugar modifications can be combined with the 5′ terminal modifications described above to further enhance miRNA activities.

In some embodiments, the complementary strand is designed so that nucleotides in the 3′ end of the complementary strand are not complementary to the active strand. This results in double-strand hybrid RNAs that are stable at the 3′ end of the active strand but relatively unstable at the 5′ end of the active strand. This difference in stability enhances the uptake of the active strand by the miRNA pathway, while reducing uptake of the complementary strand, thereby enhancing miRNA activity.

Small nucleic acid and/or antisense constructs of the methods and compositions presented herein can be delivered, for example, as an expression plasmid which, when transcribed in the cell, produces RNA which is complementary to at least a unique portion of cellular nucleic acids (e.g., small RNAs, mRNA, and/or genomic DNA). Alternatively, the small nucleic acid molecules can produce RNA which encodes mRNA, miRNA, pre-miRNA, pri-miRNA, miRNA*, anti-miRNA, or a miRNA binding site, or a variant thereof. For example, selection of plasmids suitable for expressing the miRNAs, methods for inserting nucleic acid sequences into the plasmid, and methods of delivering the recombinant plasmid to the cells of interest are within the skill in the art. See, for example, Zeng et al. (2002) Mol. Cell 9:1327-1333; Tuschl (2002), Nat. Biotechnol. 20:446-448; Brummelkamp et al. (2002) Science 296:550-553; Miyagishi et al. (2002) Nat. Biotechnol. 20:497-500; Paddison et al. (2002) Genes Dev. 16:948-958; Lee et al. (2002) Nat. Biotechnol. 20:500-505; and Paul et al. (2002) Nat. Biotechnol. 20:505-508, the entire disclosures of which are herein incorporated by reference.

Alternatively, small nucleic acids and/or antisense constructs are oligonucleotide probes that are generated ex vivo and which, when introduced into the cell, results in hybridization with cellular nucleic acids. Such oligonucleotide probes are preferably modified oligonucleotides that are resistant to endogenous nucleases, e.g., exonucleases and/or endonucleases, and are therefore stable in vivo. Exemplary nucleic acid molecules for use as small nucleic acids and/or antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches may involve the design of oligonucleotides (either DNA or RNA) that are complementary to cellular nucleic acids (e.g., complementary to biomarkers listed in Tables 1-5). Absolute complementarity is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a nucleic acid (e.g., RNA) it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner (1994) Nature 372:333). Therefore, oligonucleotides complementary to either the 5′ or 3′ untranslated, non-coding regions of genes could be used in an antisense approach to inhibit translation of endogenous mRNAs. Oligonucleotides complementary to the 5′ untranslated region of the mRNA may include the complement of the AUG start codon. Antisense oligonucleotides complementary to mRNA coding regions are less efficient inhibitors of translation but could also be used in accordance with the methods and compositions presented herein. Whether designed to hybridize to the 5′, 3′ or coding region of cellular mRNAs, small nucleic acids and/or antisense nucleic acids should be at least six nucleotides in length, and can be less than about 1000, 900, 800, 700, 600, 500, 400, 300, 200, 100, 50, 40, 30, 25, 24, 23, 22, 21, 20, 19, 18, 17, 16, 15, or 10 nucleotides in length.

Regardless of the choice of target sequence, it is preferred that in vitro studies are first performed to quantitate the ability of the antisense oligonucleotide to inhibit gene expression. In one embodiment, these studies utilize controls that distinguish between antisense gene inhibition and nonspecific biological effects of oligonucleotides. In another embodiment, these studies compare levels of the target nucleic acid or protein with that of an internal control nucleic acid or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

Small nucleic acids and/or antisense oligonucleotides can be DNA or RNA or chimeric mixtures or derivatives or modified versions thereof, single-stranded or double-stranded. Small nucleic acids and/or antisense oligonucleotides can be modified at the base moiety, sugar moiety, or phosphate backbone, for example, to improve stability of the molecule, hybridization, etc., and may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. U.S.A. 84:648-652; PCT Publication No. WO88/09810) or the blood-brain barrier (see, e.g., PCT Publication No. WO89/10134), hybridization-triggered cleavage agents. (See, e.g., Krol et al. (1988) BioTech. 6:958-976) or intercalating agents. (See, e.g., Zon (1988) Pharm. Res. 5:539549). To this end, small nucleic acids and/or antisense oligonucleotides may be conjugated to another molecule, e.g., a peptide, hybridization triggered cross-linking agent, transport agent, hybridization-triggered cleavage agent, etc.

Small nucleic acids and/or antisense oligonucleotides may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Small nucleic acids and/or antisense oligonucleotides may also comprise at least one modified sugar moiety selected from the group including but not limited to arabinose, 2-fluoroarabinose, xylulose, and hexose.

In certain embodiments, a compound comprises an oligonucleotide (e.g., a miRNA or miRNA encoding oligonucleotide) conjugated to one or more moieties which enhance the activity, cellular distribution or cellular uptake of the resulting oligonucleotide. In certain such embodiments, the moiety is a cholesterol moiety (e.g., antagomirs) or a lipid moiety or liposome conjugate. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to the oligonucleotide. In certain embodiments, a conjugate group is attached to the oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl. In certain such embodiments, the compound comprises the oligonucleotide having one or more stabilizing groups that are attached to one or both termini of the oligonucleotide to enhance properties such as, for example, nuclease stability. Included in stabilizing groups are cap structures. These terminal modifications protect the oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Suitable cap structures include a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threo-pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

Small nucleic acids and/or antisense oligonucleotides can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, small nucleic acids and/or antisense oligonucleotides comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof. In a further embodiment, small nucleic acids and/or antisense oligonucleotides are α-anomeric oligonucleotides. An α-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-0-methylribonucleotide (Inoue et al. (1987) Nucl. Acids Res. 15:61316148), or a chimeric RNA-DNA analogue (Inoue et al. (1987) FEBS Lett. 215:327-330).

Small nucleic acids and/or antisense oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451), etc. For example, an isolated miRNA can be chemically synthesized or recombinantly produced using methods known in the art. In some instances, miRNA are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGenes (Ashland, Mass., U.S.A.), Cruachem (Glasgow, UK), and Exiqon (Vedbaek, Denmark).

Small nucleic acids and/or antisense oligonucleotides can be delivered to cells in vivo. A number of methods have been developed for delivering small nucleic acids and/or antisense oligonucleotides DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

In one embodiment, small nucleic acids and/or antisense oligonucleotides may comprise or be generated from double stranded small interfering RNAs (siRNAs), in which sequences fully complementary to cellular nucleic acids (e.g. mRNAs) sequences mediate degradation or in which sequences incompletely complementary to cellular nucleic acids (e.g., mRNAs) mediate translational repression when expressed within cells, or piwiRNAs. In another embodiment, double stranded siRNAs can be processed into single stranded antisense RNAs that bind single stranded cellular RNAs (e.g., microRNAs) and inhibit their expression. RNA interference (RNAi) is the process of sequence-specific, post-transcriptional gene silencing in animals and plants, initiated by double-stranded RNA (dsRNA) that is homologous in sequence to the silenced gene. in vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. (2001) Nature 411:494-498). Accordingly, translation of a gene in a cell can be inhibited by contacting the cell with short double stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nat. Biotechnol. 20:1006; and Brummelkamp et al. (2002) Science 296:550). Vectors that can be used are commercially available, e.g., from OligoEngine under the name pSuper RNAi System™.

Ribozyme molecules designed to catalytically cleave cellular mRNA transcripts can also be used to prevent translation of cellular mRNAs and expression of cellular polypeptides, or both (See, e.g., PCT International Publication WO90/11364, published Oct. 4, 1990; Sarver et al. (1990) Science 247:1222-1225 and U.S. Pat. No. 5,093,246). While ribozymes that cleave mRNA at site specific recognition sequences can be used to destroy cellular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well-known in the art and is described more fully in Haseloff and Gerlach (1988) Nature 334:585-591. The ribozyme may be engineered so that the cleavage recognition site is located near the 5′ end of cellular mRNAs; i.e., to increase efficiency and minimize the intracellular accumulation of non-functional mRNA transcripts.

The ribozymes of the methods presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al. (1984) Science 224:574-578; Zaug et al. (1986) Science 231:470-475; Zaug et al. (1986) Nature 324:429-433; WO 88/04300; and Been et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes unlike antisense molecules, are catalytic, a lower intracellular concentration is required for efficiency.

Nucleic acid molecules to be used in triple helix formation for the inhibition of transcription of cellular genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex. Small nucleic acids (e.g., miRNAs, pre-miRNAs, pri-miRNAs, miRNA*, anti-miRNA, or a miRNA binding site, or a variant thereof), antisense oligonucleotides, ribozymes, and triple helix molecules of the methods and compositions presented herein may be prepared by any method known in the art for the synthesis of DNA and RNA molecules. These include techniques for chemically synthesizing oligodeoxyribonucleotides and oligoribonucleotides well-known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. One of skill in the art will readily understand that polypeptides, small nucleic acids, and antisense oligonucleotides can be further linked to another peptide or polypeptide (e.g., a heterologous peptide), e.g., that serves as a means of protein detection. Non-limiting examples of label peptide or polypeptide moieties useful for detection in the invention include, without limitation, suitable enzymes such as horseradish peroxidase, alkaline phosphatase, beta-galactosidase, or acetylcholinesterase; epitope tags, such as FLAG, MYC, HA, or HIS tags; fluorophores such as green fluorescent protein; dyes; radioisotopes; digoxygenin; biotin; antibodies; polymers; as well as others known in the art, for example, in Principles of Fluorescence Spectroscopy, Joseph R. Lakowicz (Editor), Plenum Pub Corp, 2nd edition (July 1999).

The modulatory agents described herein (e.g., antibodies, small molecules, peptides, fusion proteins, or small nucleic acids) can be incorporated into pharmaceutical compositions and administered to a subject in vivo. The compositions may contain a single such molecule or agent or any combination of agents described herein. “Single active agents” described herein can be combined with other pharmacologically active compounds (“second active agents”) known in the art according to the methods and compositions provided herein.

The production and use of biomarker nucleic acid and/or biomarker polypeptide molecules described herein can be facilitated by using standard recombinant techniques. In some embodiments, such techniques use vectors, preferably expression vectors, containing a nucleic acid encoding a biomarker polypeptide or a portion of such a polypeptide. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the present invention is intended to include such other forms of expression vectors, such as viral vectors (e.g., replication defective retroviruses, adenoviruses and adeno-associated viruses), which serve equivalent functions.

The recombinant expression vectors of the present invention comprise a nucleic acid of the present invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the present invention can be introduced into host cells to thereby produce proteins or peptides, including fusion proteins or peptides, encoded by nucleic acids as described herein.

The recombinant expression vectors for use in the present invention can be designed for expression of a polypeptide corresponding to a marker of the present invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology: Methods in Enzymology vol. 185, Academic Press, San Diego, Calif., 1991). Target biomarker nucleic acid expression from the pTrc vector relies on host RNA polymerase transcription from a hybrid trp-lac fusion promoter. Target biomarker nucleic acid expression from the pET 11d vector relies on transcription from a T7 gn10-lac fusion promoter mediated by a co-expressed viral RNA polymerase (T7 gn1). This viral polymerase is supplied by host strains BL21 (DE3) or HMS174 (DE3) from a resident prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman, p. 119-128, In Gene Expression Technology: Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the present invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid of the present invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra. In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. U.S.A. 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The present invention further provides a recombinant expression vector comprising a DNA molecule cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the present invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes (see Weintraub et al., 1986, Trends in Genetics, Vol. 1(1)).

Another aspect of the present invention pertains to host cells into which a recombinant expression vector of the present invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

### V. Analyzing Biomarker Nucleic Acids and Polypeptides

Biomarker nucleic acids and/or biomarker polypeptides can be analyzed according to the methods described herein and techniques known to the skilled artisan to identify such genetic or expression alterations useful for the present invention including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like. a. Methods for Detection of Copy Number

Methods of evaluating the copy number of a biomarker nucleic acid are well-known to those of skill in the art. The presence or absence of chromosomal gain or loss can be evaluated simply by a determination of copy number of the regions or markers identified herein.

In one embodiment, a biological sample is tested for the presence of copy number changes in genomic loci containing the genomic marker. A copy number of at least 3, 4, 5, 6, 7, 8, 9, or 10 is predictive of poorer outcome of inhibitor(s) of one or more biomarkers listed in Tables 1-5, and immunotherapy combination treatments.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well-known in the art to detect RNA can be used, such that higher or lower expression relative to an appropriate control (e.g., a non-amplified portion of the same or related cell tissue, organ, etc.) provides an estimate of the relative copy number of the target nucleic acid. An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome.

Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well-known in the art (see, e.g., U.S. Pat. Nos. 6,335,167; 6,197,501; 5,830,645; and 5,665,549 and Albertson (1984)EMBO J. 3: 1227-1234; Pinkel (1988) Proc. Natl. Acad. Sci. U.S.A. 85: 9138-9142; EPO Pub. No. 430,402; Methods in Molecular Biology, Vol. 33: In situ Hybridization Protocols, Choo, ed., Humana Press, Totowa, N.J. (1994), etc.) In another embodiment, the hybridization protocol of Pinkel, et al. (1998) Nature Genetics 20: 207211, or of Kallioniemi (1992) Proc. Natl Acad Sci U.S.A. 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the present invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. U.S.A. 86: 1173), self-sustained sequence replication (Guatelli, et al. (1990) Proc. Nat. Acad. Sci. U.S.A. 87: 1874), dot PCR, and linker adapter PCR, etc.

Loss of heterozygosity (LOH) and major copy proportion (MCP) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93; Li et al., (2008) MBC Bioinform. 9, 204-219) may also be used to identify regions of amplification or deletion.

b. Methods for Detection of Biomarker Nucleic Acid Expression

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J Path. 154: 61 and Murakami et al. (2000) Kidney Int. 58:1346). For example, Murakami et al., supra, describe isolation of a cell from a previously immunostained tissue section.

It is also possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin. The RNA sample can then be enriched in particular species. In one embodiment, poly(A)+RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc. Natl. Acad. Sci. U.S.A. 86: 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used.

Other known amplification methods which can be utilized herein include but are not limited to the so-called “NASBA” or “3SR” technique described in PNAS U.S.A. 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. U.S.A., 87, 1874 (1990)); and transcription amplification (see, e.g., Kwoh et al., Proc. Natl. Acad. Sci. U.S.A. 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos. 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ¹⁵S. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

c. Methods for Detection of Biomarker Protein Expression

The activity or level of a biomarker protein can be detected and/or quantified by detecting or quantifying the expressed polypeptide. The polypeptide can be detected and quantified by any of a number of means well-known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to inhibitor(s) of one or more biomarkers listed in Tables 1-5, and immunotherapy combination treatments. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins. Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

d. Methods for Detection of Biomarker Structural Alterations

The following illustrative methods can be used to identify the presence of a structural alteration in a biomarker nucleic acid and/or biomarker polypeptide molecule in order to, for example, identify the one or more biomarkers listed in Tables 1-5, or other biomarkers used in the immunotherapies described herein that are overexpressed, overfunctional, and the like.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. U.S.A. 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. U.S.A. 87:1874-1878), transcriptional amplification system (Kwoh, D. Y. et al. (1989) Proc. Natl. Acad. Sci. U.S.A. 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

In an alternative embodiment, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such biomarker genetic mutations can be identified in a variety of contexts, including, for example, germline and somatic mutations.

In yet another embodiment, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. U.S.A. 74:560 or Sanger (1977) Proc. Natl. Acad Sci. U.S.A. 74:5463. It is also contemplated that any of a variety of automated sequencing procedures can be utilized when performing the diagnostic assays (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. U.S.A. 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039.)

In other embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad. Sci U.S.A. 86:2766; see also Cotton (1993) Mutat. Res. 285:125-144 and Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. U.S.A. 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci U.S.A. 88:189). In such cases, ligation will occur only if there is a perfect match at the 3′ end of the 5′ sequence making it possible to detect the presence of a known mutation at a specific site by looking for the presence or absence of amplification.

### VI. Anti-Cancer Therapies

The efficacy of inhibitors of one or more biomarkers listed in Tables 1-5 and immunotherapy combination treatment is predicted according to biomarker amount and/or activity associated with a cancer in a subject according to the methods described herein. In one embodiment, such inhibitor and immunotherapy combination treatments (e.g., one or more inhibitor and immunotherapy combination treatment in combination with one or more additional anti-cancer therapies, such as another immune checkpoint inhibitor) can be administered, particularly if a subject has first been indicated as being a likely responder to inhibitor and immunotherapy combination treatment. In another embodiment, such inhibitor and immunotherapy combination treatment can be avoided once a subject is indicated as not being a likely responder to inhibitor and immunotherapy combination treatment and an alternative treatment regimen, such as targeted and/or untargeted anti-cancer therapies can be administered.

Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with anti-immune checkpoint therapy. In addition, any representative embodiment of an agent to modulate a particular target can be adapted to any other target described herein by the ordinarily skilled artisan.

The term “targeted therapy” refers to administration of agents that selectively interact with a chosen biomolecule to thereby treat cancer. One example includes immunotherapies such as immune checkpoint inhibitors, which are well-known in the art. For example, anti-PD-1 pathway agents, such as therapeutic monoclonal blocking antibodies, which are well-known in the art and described above, can be used to target tumor microenvironments and cells expressing unwanted components of the PD-1 pathway, such as PD-1, PD-L1, and/or PD-L2.

For example, the term “PD-1 pathway” refers to the PD-1 receptor and its ligands, PD-L1 and PD-L2. “PD-1 pathway inhibitors” block or otherwise reduce the interaction between PD-1 and one or both of its ligands such that the immunoinhibitory signaling otherwise generated by the interaction is blocked or otherwise reduced. Anti-immune checkpoint inhibitors can be direct or indirect. Direct anti-immune checkpoint inhibitors block or otherwise reduce the interaction between an immune checkpoint and at least one of its ligands. For example, PD-1 inhibitors can block PD-1 binding with one or both of its ligands. Direct PD-1 combination inhibitors are well-known in the art, especially since the natural binding partners of PD-1 (e.g., PD-L1 and PD-L2), PD-L1 (e.g., PD-1 and B7-1), and PD-L2 (e.g., PD-1 and RGMb) are known.

For example, agents which directly block the interaction between PD-1 and PD-L1, PD-1 and PD-L2, PD-1 and both PD-L1 and PD-L2, such as a bispecific antibody, can prevent inhibitory signaling and upregulate an immune response (i.e., as a PD-1 pathway inhibitor). Alternatively, agents that indirectly block the interaction between PD-1 and one or both of its ligands can prevent inhibitory signaling and upregulate an immune response. For example, B7-1 or a soluble form thereof, by binding to a PD-L1 polypeptide indirectly reduces the effective concentration of PD-L1 polypeptide available to bind to PD-1. Exemplary agents include monospecific or bispecific blocking antibodies against PD-1, PD-L1, and/or PD-L2 that block the interaction between the receptor and ligand(s); a non-activating form of PD-1, PD-L1, and/or PD-L2 (e.g., a dominant negative or soluble polypeptide), small molecules or peptides that block the interaction between PD-1, PD-L1, and/or PD-L2; fusion proteins (e.g. the extracellular portion of PD-1, PD-L1, and/or PD-L2, fused to the Fc portion of an antibody or immunoglobulin) that bind to PD-1, PD-L1, and/or PD-L2 and inhibit the interaction between the receptor and ligand(s); a non-activating form of a natural PD-1, PD-L2, and/or PD-L2 ligand, and a soluble form of a natural PD-1, PD-L2, and/or PD-L2 ligand.

Indirect anti-immune checkpoint inhibitors block or otherwise reduce the immunoinhibitory signaling generated by the interaction between the immune checkpoint and at least one of its ligands. For example, an inhibitor can block the interaction between PD-1 and one or both of its ligands without necessarily directly blocking the interaction between PD-1 and one or both of its ligands. For example, indirect inhibitors include intrabodies that bind the intracellular portion of PD-1 and/or PD-L1 required to signal to block or otherwise reduce the immunoinhibitory signaling. Similarly, nucleic acids that reduce the expression of PD-1, PD-L1, and/or PD-L2 can indirectly inhibit the interaction between PD-1 and one or both of its ligands by removing the availability of components for interaction. Such nucleic acid molecules can block PD-L1, PD-L2, and/or PD-L2 transcription or translation.

Similarly, agents which directly block the interaction between one or more biomarkers listed in Tables 1-5, and the binding partners and/or substrates of such one or more biomarkers, and the like, can remove the inhibition to such one or more biomarkers-regulated signaling and its downstream immune responses, such as increasing sensitivity to interferon signaling.

Alternatively, agents that indirectly block the interaction between such one or more biomarkers and its binding partners/substrates can remove the inhibition to such one or more biomarkers-regulated signaling and its downstream immune responses. For example, a truncated or dominant negative form of such one or more biomarkers, such as biomarker fragments without functional activity, by binding to a substrate of such one or more biomarkers and indirectly reducing the effective concentration of such substrate available to bind to the one or more biomarkers in cell. Exemplary agents include monospecific or bispecific blocking antibodies, especially intrabodies, against the one or more biomarkers and/or their substrate(s) that block the interaction between the one or more biomarkers and their substrate(s); a non-active form of such one or more biomarkers and/or their substrate(s) (e.g., a dominant negative polypeptide), small molecules or peptides that block the interaction between such one or more biomarkers and their substrate(s) or the activity of such one or more biomarkers; and a non-activating form of a natural biomarker and/or its substrate(s).

Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In one embodiment, immunotherapy comprises adoptive cell-based immunotherapies. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, patient-specific neoantigen vaccines, and the like.

In another embodiment, immunotherapy comprises non-cell-based immunotherapies. In one embodiment, compositions comprising antigens with or without vaccine-enhancing adjuvants are used. Such compositions exist in many well-known forms, such as peptide compositions, oncolytic viruses, recombinant antigen comprising fusion proteins, and the like. In still another embodiment, immunomodulatory interleukins, such as IL-2, IL-6, IL-7, IL-12, IL-17, IL-23, and the like, as well as modulators thereof (e.g., blocking antibodies or more potent or longer lasting forms) are used. In yet another embodiment, immunomodulatory cytokines, such as interferons, G-CSF, imiquimod, TNFalpha, and the like, as well as modulators thereof (e.g., blocking antibodies or more potent or longer lasting forms) are used. In another embodiment, immunomodulatory chemokines, such as CCL3, CCL26, and CXCL7, and the like, as well as modulators thereof (e.g., blocking antibodies or more potent or longer lasting forms) are used. In another embodiment, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

In still another embodiment, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C (indole-3-carbinol)/DIM(di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereto, are used. In yet another embodiment, immunomodulatory antibodies or proteins are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 an antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like. Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, agents and therapies other than immunotherapy or in combination thereof can be used in combination with inhibitors of one or more biomarkers listed in Tables 1-5, with or without immunotherapies to stimulate an immune response to thereby treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

The term “untargeted therapy” refers to administration of agents that do not selectively interact with a chosen biomolecule yet treat cancer. Representative examples of untargeted therapies include, without limitation, chemotherapy, gene therapy, and radiation therapy. In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of β-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research Fundamental and Molecular Mechanisms of Mutagenesis, Volume 477, Number 1, 2 Jun. 2001, pp. 97110(14)). Poly(ADP-ribose) polymerase 1 (PARP1) is a key molecule in the repair of DNA single-strand breaks (SSBs) (de Murcia J. et al. 1997. Proc Natl Acad Sci U.S.A. 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, surgical intervention can occur to physically remove cancerous cells and/or tissues. In still another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH—RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In yet another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early non-small cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter-less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The duration and/or dose of treatment with therapies may vary according to the particular therapeutic agent or combination thereof. An appropriate treatment time for a particular cancer therapeutic agent will be appreciated by the skilled artisan. The present invention contemplates the continued assessment of optimal treatment schedules for each cancer therapeutic agent, where the phenotype of the cancer of the subject as determined by the methods of the present invention is a factor in determining optimal treatment doses and schedules.

Any means for the introduction of a polynucleotide into mammals, human or non-human, or cells thereof may be adapted to the practice of this invention for the delivery of the various constructs of the present invention into the intended recipient. In one embodiment of the present invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al., Am J Respir Cell Mol Biol 10:24-29, 1994; Tsan et al., Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well-known and any can be selected for a particular application. In one embodiment of the present invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. U.S.A. 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. U.S.A. 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805). Other viral vector systems that can be used to deliver a polynucleotide of the present invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth, Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J. Virol., 64:642-650).

In other embodiments, target DNA in the genome can be manipulated using well-known methods in the art. For example, the target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

In other embodiments, recombinant biomarker polypeptides, and fragments thereof, can be administered to subjects. In some embodiments, fusion proteins can be constructed and administered which have enhanced biological properties. In addition, the biomarker polypeptides, and fragment thereof, can be modified according to well-known pharmacological methods in the art (e.g., pegylation, glycosylation, oligomerization, etc.) in order to further enhance desirable biological activities, such as increased bioavailability and decreased proteolytic degradation.

### VII. Clinical Efficacy

Clinical efficacy can be measured by any method known in the art. For example, the response to a therapy, such as inhibitors of one or more biomarkers listed in Tables 1-5, and immunotherapy combination treatment, relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to immunotherapies, such as anti-immune checkpoint therapies, are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any immunotherapy, such as anti-immune checkpoint therapy. The outcome measurement may be pathologic response to therapy given in the neoadjuvant setting. Alternatively, outcome measures, such as overall survival and disease-free survival can be monitored over a period of time for subjects following immunotherapies for whom biomarker measurement values are known. In certain embodiments, the same doses of immunotherapy agents, if any, are administered to each subject. In related embodiments, the doses administered are standard doses known in the art for those agents used in immunotherapies. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of an immunotherapy can be determined using methods such as those described in the Examples section.

### VIII. Further Uses and Methods of the Present Invention

The compositions described herein can be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

a. Screening Methods One aspect of the present invention relates to screening assays, including non-cell based assays and xenograft animal model assays. In one embodiment, the assays provide a method for identifying whether a cancer is likely to respond to inhibitors of one or more biomarkers listed in Tables 1-5, and immunotherapy combination treatments, such as in a human by using a xenograft animal model assay, and/or whether an agent can inhibit the growth of or kill a cancer cell that is unlikely to respond to inhibitors of one or more biomarkers listed in Tables 1-5, and immunotherapy combination treatments.

In one embodiment, the present invention relates to assays for screening test agents which bind to, or modulate the biological activity of, at least one biomarker described herein (e.g., in the tables, figures, examples, or otherwise in the specification). In one embodiment, a method for identifying such an agent entails determining the ability of the agent to modulate, e.g. inhibit, the at least one biomarker described herein.

In one embodiment, an assay is a cell-free or cell-based assay, comprising contacting at least one biomarker described herein, with a test agent, and determining the ability of the test agent to modulate (e.g., inhibit) the enzymatic activity of the biomarker, such as by measuring direct binding of substrates or by measuring indirect parameters as described below.

For example, in a direct binding assay, biomarker protein (or their respective target polypeptides or molecules) can be coupled with a radioisotope or enzymatic label such that binding can be determined by detecting the labeled protein or molecule in a complex. For example, the targets can be labeled with ¹²⁵, ³⁵S, ¹⁴C, or ³H, either directly or indirectly, and the radioisotope detected by direct counting of radioemission or by scintillation counting. Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies described herein can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to modulate the interaction between the biomarker and a substrate or a biomarker and its natural binding partner can be accomplished by determining the ability of the test agent to modulate the activity of a polypeptide or other product that functions downstream or upstream of its position within the signaling pathway (e.g., feedback loops). Such feedback loops are well-known in the art (see, for example, Chen and Guillemin (2009) Int. J. Tryptophan Res. 2:1-19).

The present invention further pertains to novel agents identified by the above-described screening assays. Accordingly, it is within the scope of this invention to further use an agent identified as described herein, such as in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

b. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the amount and/or activity level of a biomarker described herein in the context of a biological sample (e.g., blood, serum, cells, or tissue) to thereby determine whether an individual afflicted with a cancer is likely to respond to inhibitors of one or more biomarkers listed in Tables 1-5, and immunotherapy combination treatments, such as in a cancer. Such assays can be used for prognostic or predictive purpose alone, or can be coupled with a therapeutic intervention to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers described herein, such as those in the tables, figures, examples, and otherwise described in the specification.

Another aspect of the present invention pertains to monitoring the influence of agents (e.g., drugs, compounds, and small nucleic acid-based molecules) on the expression or activity of a biomarker described herein. These and other agents are described in further detail in the following sections.

The skilled artisan will also appreciated that, in certain embodiments, the methods of the present invention implement a computer program and computer system. For example, a computer program can be used to perform the algorithms described herein. A computer system can also store and manipulate data generated by the methods of the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods of the present invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the noncancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

c. Diagnostic Assays

The present invention provides, in part, methods, systems, and code for accurately classifying whether a biological sample is associated with a cancer that is likely to respond to inhibitors of one or more biomarkers listed in Tables 1-5, and immunotherapy combination treatments. In some embodiments, the present invention is useful for classifying a sample (e.g., from a subject) as associated with or at risk for responding to or not responding to inhibitors of one or more biomarkers listed in Tables 1-5, and immunotherapy combination treatments using a statistical algorithm and/or empirical data (e.g., the amount or activity of a biomarker described herein, such as in the tables, figures, examples, and otherwise described in the specification).

An exemplary method for detecting the amount or activity of a biomarker described herein, and thus useful for classifying whether a sample is likely or unlikely to respond to inhibitors of one or more biomarkers listed in Tables 1-5, and immunotherapy combination treatments involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a base upon a prediction or probability value and the presence or level of the biomarker. The use of a single learning statistical classifier system typically classifies the sample as, for example, a likely immunotherapy responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In another embodiment, the diagnosis of a subject is followed by administering to the individual a therapeutically effective amount of a defined treatment based upon the diagnosis.

In one embodiment, the methods further involve obtaining a control biological sample (e.g., biological sample from a subject who does not have a cancer or whose cancer is susceptible to inhibitors of one or more biomarkers listed in Tables 1-5, and immunotherapy combination treatments), a biological sample from the subject during remission, or a biological sample from the subject during treatment for developing a cancer progressing despite inhibitors of one or more biomarkers listed in Tables 1-5, and immunotherapy combination treatments.

d. Prognostic Assays

The diagnostic methods described herein can furthermore be utilized to identify subjects having or at risk of developing a cancer that is likely or unlikely to be responsive to inhibitors of one or more biomarkers listed in Tables 1-5, and immunotherapy combination treatments. The assays described herein, such as the preceding diagnostic assays or the following assays, can be utilized to identify a subject having or at risk of developing a disorder associated with a misregulation of the amount or activity of at least one biomarker described herein, such as in cancer. Alternatively, the prognostic assays can be utilized to identify a subject having or at risk for developing a disorder associated with a misregulation of the at least one biomarker described herein, such as in cancer. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with the aberrant biomarker expression or activity.

e. Treatment Methods

The therapeutic compositions described herein, such as the combination of inhibitors of one or more biomarkers listed in Tables 1-5, and immunotherapy, can be used in a variety of in vitro and in vivo therapeutic applications using the formulations and/or combinations described herein. In one embodiment, the therapeutic agents can be used to treat cancers determined to be responsive thereto. For example, single or multiple agents that inhibit or block both an inhibitor of one or more biomarkers listed in Tables 1-5, and an immunotherapy can be used to treat cancers in subjects identified as likely responders thereto.

Modulatory methods of the present invention involve contacting a cell, such as an immune cell with an agent that inhibits or blocks the expression and/or activity of such one or more biomarkers and an immunotherapy, such as an immune checkpoint inhibitor (e.g., PD-1). Exemplary agents useful in such methods are described above. Such agents can be administered in vitro or ex vivo (e.g., by contacting the cell with the agent) or, alternatively, in vivo (e.g., by administering the agent to a subject). As such, the present invention provides methods useful for treating an individual afflicted with a condition that would benefit from an increased immune response, such as an infection or a cancer like colorectal cancer.

Agents that upregulate immune responses, which can be in the form of enhancing an existing immune response or eliciting an initial immune response. Thus, enhancing an immune response using the subject compositions and methods is useful for treating cancer but can also be useful for treating an infectious disease (e.g., bacteria, viruses, or parasites), a parasitic infection, and an immunosuppressive disease.

Exemplary infectious disorders include viral skin diseases, such as Herpes or shingles, in which case such an agent can be delivered topically to the skin. In addition, systemic viral diseases, such as influenza, the common cold, and encephalitis might be alleviated by systemic administration of such agents. In one preferred embodiment, agents that upregulate the immune response described herein are useful for modulating the arginase/iNOS balance during Trypanosoma cruzi infection in order to facilitate a protective immune response against the parasite.

Immune responses can also be enhanced in an infected patient through an ex vivo approach, for instance, by removing immune cells from the patient, contacting immune cells in vitro with an agent described herein and reintroducing the in vitro stimulated immune cells into the patient.

In certain instances, it may be desirable to further administer other agents that upregulate immune responses, for example, forms of other B7 family members that transduce signals via costimulatory receptors, in order to further augment the immune response. Such additional agents and therapies are described further below. Agents that upregulate an immune response can be used prophylactically in vaccines against various polypeptides (e.g., polypeptides derived from pathogens). Immunity against a pathogen (e.g., a virus) can be induced by vaccinating with a viral protein along with an agent that upregulates an immune response, in an appropriate adjuvant.

In another embodiment, upregulation or enhancement of an immune response function, as described herein, is useful in the induction of tumor immunity.

In another embodiment, the immune response can be stimulated by the methods described herein, such that preexisting tolerance, clonal deletion, and/or exhaustion (e.g., T cell exhaustion) is overcome. For example, immune responses against antigens to which a subject cannot mount a significant immune response, e.g., to an autologous antigen, such as a tumor specific antigens can be induced by administering appropriate agents described herein that upregulate the immune response. In one embodiment, an autologous antigen, such as a tumor-specific antigen, can be coadministered. In another embodiment, the subject agents can be used as adjuvants to boost responses to foreign antigens in the process of active immunization.

In one embodiment, immune cells are obtained from a subject and cultured ex vivo in the presence of an agent as described herein, to expand the population of immune cells and/or to enhance immune cell activation. In a further embodiment the immune cells are then administered to a subject. Immune cells can be stimulated in vitro by, for example, providing to the immune cells a primary activation signal and a costimulatory signal, as is known in the art. Various agents can also be used to costimulate proliferation of immune cells. In one embodiment immune cells are cultured ex vivo according to the method described in PCT Application No. WO 94/29436. The costimulatory polypeptide can be soluble, attached to a cell membrane, or attached to a solid surface, such as a bead.

### IX. Administration of Agents

The immune modulating agents encompassed by the present invention are administered to subjects in a biologically compatible form suitable for pharmaceutical administration in vivo, to enhance immune cell mediated immune responses. By “biologically compatible form suitable for administration in vivo” is meant a form to be administered in which any toxic effects are outweighed by the therapeutic effects. The term “subject” is intended to include living organisms in which an immune response can be elicited, e.g., mammals. Examples of subjects include humans, dogs, cats, mice, rats, and transgenic species thereof. Administration of an agent as described herein can be in any pharmacological form including a therapeutically active amount of an agent alone or in combination with a pharmaceutically acceptable carrier.

Administration of a therapeutically active amount of the therapeutic composition of the present invention is defined as an amount effective, at dosages and for periods of time necessary, to achieve the desired result. For example, a therapeutically active amount of an agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

Inhibiting or blocking expression and/or activity of one or more biomarkers listed in Tables 1-5, alone or in combination with an immunotherapy, can be accomplished by combination therapy with the modulatory agents described herein. Combination therapy describes a therapy in which one or more biomarkers are inhibited or blocked with an immunotherapy simultaneously. This may be achieved by administration of the modulatory agent described herein with the immunotherapy simultaneously (e.g., in a combination dosage form or by simultaneous administration of single agents) or by administration of single inhibitory agent for such one or more biomarkers and the immunotherapy, according to a schedule that results in effective amounts of each modulatory agent present in the patient at the same time.

The therapeutic agents described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity, or expression and/or activity of the complex encompassed by the present invention. These salts can be prepared in situ during the final isolation and purification of the therapeutic agents, or by separately reacting a purified therapeutic agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression and/or activity, or expression and/or activity of the complex. These salts can likewise be prepared in situ during the final isolation and purification of the therapeutic agents, or by separately reacting the purified therapeutic agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra). Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well-known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a therapeutic agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a therapeutic agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well-known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more therapeutic agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate. Dosage forms for the topical or transdermal administration of an agent that modulates (e.g., inhibits) biomarker expression and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel. Ophthalmic formulations, eye ointments, powders, solutions and the like, are also contemplated as being within the scope of this invention.

Pharmaceutical compositions of this invention suitable for parenteral administration comprise one or more therapeutic agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the therapeutic agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The nucleic acid molecules of the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. U.S.A. 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In one embodiment, an agent encompassed by the present invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays.

### X. Isolated Modified Polypeptides and Complexes

The present invention relates, in part, to an isolated polypeptide and/or a complex comprising the same, such as those selected from the group consisting of polypeptides listed in Tables 1-5. In some embodiments, the complex is a Polycomb repressor complex (e.g., a PRC1.1 complex).

Complexes for use according to the present invention can be single polypeptides (e.g., USP7 polypeptide or fragment thereof) in association with another moiety or combinations of polypeptides (e.g., protein complexes comprising a USP7 subunit) in association with each other and/or in association with another moiety.

In one aspect of the present invention, a composition is provided comprising a complex of polypeptides comprising at least one variant polypeptide. In some embodiments, the variant polypeptide is a mutant peptide that has an amino acid sequence comprising at least one variant amino acid residue relative to a wildtype amino acid sequence. In some embodiment, the variant polypeptide is a wildtype polypeptide in a species that is different from the species from which the other polypeptides in the complex are derived. In certain embodiments, the isolated polypeptide is of the fragment comprising a wildtype or a domain that is modified relative to the wild-type sequence. In some embodiments, the isolated modified polypeptide fragment has reduced activity as compared to the wild-type fragment. In some embodiments, the isolated modified fragment has one or more of the following compared to the wild-type fragment: a. replacement of at least one basic amino acid for a neutral or an acidic amino acid, optionally wherein the basic amino acid is an outward-facing residue of the alpha helix; b. deletion of at least one basic amino acid, optionally wherein the basic amino acid is an outward-facing residue of the alpha helix; or c. reduced isoelectric point, reduced charge potential, and/or reduced net positive charge. In some embodiments, the isolated fragment further comprises a heterologous amino acid sequence, such as an affinity tag or a label. Tags can include Glutathione-S-Transferase (GST), calmodulin binding protein (CBP), protein C tag, Myc tag, HaloTag, HA tag, Flag tag, His tag, biotin tag, and V5 tag. Labels can include a fluorescent protein.

In some embodiments, protein complexes comprising a modified subunit that can be a fragment as described above or a full-length polypeptide that is modified to have the functional properties of such a fragment, are provided. In certain embodiments, at least one subunit of a complex encompassed by the present invention is a homolog, a derivative, e.g., a functionally active derivative, a fragment, e.g., a functionally active fragment, of a protein subunit of a complex encompassed by the present invention. In certain embodiments encompassed by the present invention, a homolog/ortholog, derivative or fragment of a protein subunit of a complex encompassed by the present invention is still capable of forming a complex with the other subunit(s). Complex-formation can be tested by any method known to the skilled artisan. Such methods include, but are not limited to, non-denaturing PAGE, FRET, and Fluorescence Polarization Assay.

Homologs (e.g., nucleic acids encoding subunit proteins from other species) or other related sequences (e.g., paralogs) which are members of a native cellular protein complex can be identified and obtained by low, moderate or high stringency hybridization with all or a portion of the particular nucleic acid sequence as a probe, using methods well-known in the art for nucleic acid hybridization and cloning.

Exemplary moderately stringent hybridization conditions are as follows: prehybridization of filters containing DNA is carried out for 8 hours to overnight at 65° C. in buffer composed of 6×SSC, 50 mM Tris-HCI (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 min before autoradiography. Alternatively, exemplary conditions of high stringency are as follows: e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel et al., eds., (1989) Current Protocols in Molecular Biology, Vol. I, Green Publishing Associates, Inc., and John Wiley & sons, Inc., New York, at p. 2.10.3). Other conditions of high stringency which may be used are well-known in the art. Exemplary low stringency hybridization conditions comprise hybridization in a buffer comprising 35% formamide, 5×SSC, 50 mM Tris-HCI (pH 7.5), 5 mM EDTA, 0.02% PVP, 0.02% Ficoll, 0.2% BSA, 100 μg/ml denatured salmon sperm DNA, and 10% (wt/vol) dextran sulfate for 18-20 hours at 40° C., washing in a buffer consisting of 2×SSC, 25 mM Tris-HCI (pH 7.4), 5 mM EDTA, and 0.1% SDS for 1.5 hours at 55° C., and washing in a buffer consisting of 2×SSC, 25 mM Tris-HCI (pH 7.4), 5 mM EDTA, and 0.1% SDS for 1.5 hours at 60° C.

In certain embodiments, a homolog of a subunit binds to the same proteins to which the subunit binds. In certain, more specific embodiments, a homolog of a subunit binds to the same proteins to which the subunit binds wherein the binding affinity between the homolog and the binding partner of the subunit is at least 10%, at least 20%, at least 30%, at least 40%, at least 50%, at least 60%, at least 70%, at least 80%, at least 90%, at least 95% or at least 98% of the binding affinity between the subunit and the binding partner. Binding affinities between proteins can be determined by any method known to the skilled artisan.

In certain embodiments, a fragment of a protein subunit of the complex consists of at least 6 (continuous) amino acids, of at least 10, at least 20 amino acids, at least 30 amino acids, at least 40 amino acids, at least 50 amino acids, at least 75 amino acids, at least 100 amino acids, at least 150 amino acids, at least 200 amino acids, at least 250 amino acids, at least 300 amino acids, at least 400 amino acids, or at least 500 amino acids of the protein subunit of the naturally occurring protein complex. In specific embodiments. Such fragments are not larger than 40 amino acids, 50 amino acids, 75 amino acids, 100 amino acids, 150 amino acids, 200 amino acids, 250 amino acids, 300 amino acids, 400 amino acids, or than 500 amino acids. In more specific embodiments, the functional fragment is capable of forming a complex encompassed by the present invention, i.e., the fragment can still bind to at least one other protein subunit to form a complex encompassed by the present invention. In some embodiments, fragments are provided herein, which share an identical region of 30, 31, 32, 33, 34, 35, 36, 37, 38, 39, 40, 41, 42, 43, or 44 or more, or any range in between. In some embodiments, the domain can include 1, 2, 3, 4, 5, 6, 7, 8, 9, 10, or more, or any range in between, amino acid residue deletions and/or mutations as compared to the wild-type domain.

Derivatives or analogs of subunit proteins include, but are not limited, to molecules comprising regions that are substantially homologous to the subunit proteins, in various embodiments, by at least 30%, 40%, 50%, 60%, 70%, 80%, 90% or 95% identity over an amino acid sequence of identical size or when compared to an aligned sequence in which the alignment is done by a computer homology program known in the art, or whose encoding nucleic acid is capable of hybridizing to a sequence encoding the subunit protein under stringent, moderately stringent, or nonstringent conditions.

Derivatives of a protein subunit include, but are not limited to, fusion proteins of a protein subunit of a complex encompassed by the present invention to a heterologous amino acid sequence, mutant forms of a protein subunit of a complex encompassed by the present invention, and chemically modified forms of a protein subunit of a complex encompassed by the present invention. In a specific embodiment, the functional derivative of a protein subunit of a complex encompassed by the present invention is capable of forming a complex encompassed by the present invention, i.e., the derivative can still bind to at least one other protein subunit to form a complex encompassed by the present invention.

In certain embodiments encompassed by the present invention, at least two subunits of a complex encompassed by the present invention are linked to each other via at least one covalent bond. A covalent bond between subunits of a complex encompassed by the present invention increases the stability of the complex encompassed by the present invention because it prevents the dissociation of the subunits. Any method known to the skilled artisan can be used to achieve a covalent bond between at least two subunits encompassed by the present invention.

In specific embodiments, covalent cross-links are introduced between adjacent subunits. Such cross-links can be between the side chains of amino acids at opposing sides of the dimer interface. Any functional groups of amino acid residues at the dimer interface in combination with suitable cross-linking agents can be used to create covalent bonds between the protein subunits at the dimer interface. Existing amino acids at the dimer interface can be used or, alternatively, suitable amino acids can be introduced by site-directed mutagenesis.

In exemplary embodiments, cysteine residues at opposing sides of the dimer interface are oxidized to form disulfide bonds. See, e.g., Reznik et al., (1996) Nat Bio Technol 14:1007-1011, at page 1008. 1,3-dibromoacetone can also be used to create an irreversible covalent bond between two sulfhydryl groups at the dimer interface. In certain other embodiments, lysine residues at the dimer interface are used to create a covalent bond between the protein subunits of the complex. Crosslinkers that can be used to create covalent bonds between the epsilon amino groups of lysine residues are, e.g., but are not limited to, bis(sulfosuccinimidyl)suberate; dimethyladipimidate-2HD1; disuccinimidyl glutarate; N-hydroxysuccinimidyl 2,3-dibromoproprionate.

In other specific embodiments, two or more interacting subunits, or homologues, derivatives or fragments thereof, are directly fused together, or covalently linked together through a peptide linker, forming a hybrid protein having a single unbranched polypeptide chain. Thus, the protein complex may be formed by “intramolecular interactions between two portions of the hybrid protein. In still another embodiment, at least one of the fused or linked interacting subunit in this protein complex is a homologue, derivative or fragment of a native protein.

In specific embodiments, at least one subunit, or a homologue, derivative or fragment thereof, may be expressed as fusion or chimeric protein comprising the subunit, homologue, derivative or fragment, joined via a peptide bond to a heterologous amino acid sequence.

As used herein, a “chimeric protein” or “fusion protein” comprises all or part (preferably a biologically active part) of a polypeptide corresponding to a subunit or a fragment, homologue or derivative thereof, operably linked to a heterologous polypeptide (i.e., a polypeptide other than the polypeptide corresponding to the subunit or a fragment, homologue or derivative thereof). Within the fusion protein, the term “operably linked” is intended to indicate that the polypeptide encompassed by the present invention and the heterologous polypeptide are fused in-frame to each other. The heterologous polypeptide can be fused to the amino-terminus or the carboxyl-terminus of the polypeptide encompassed by the present invention.

In one embodiment, the heterologous amino acid sequence comprises an affinity tag that can be used for affinity purification. In another embodiment, the heterologous amino acid sequence includes a fluorescent label. In still another embodiment, the fusion protein contains a heterologous signal sequence, immunoglobulin fusion protein, toxin, or other useful protein sequences.

A variety of peptide tags known in the art may be used to generate fusion proteins of the protein subunits of a complex encompassed by the present invention, such as but not limited to the immunoglobulin constant regions, polyhistidine sequence (Petty, (1996) Metal-chelate affinity chromatography, in Current Protocols in Molecular Biology, Vol. 2, Ed. Ausubel et al., Greene Publish. Assoc. & Wiley Interscience), glutathione S-transferase (GST: Smith, (1993) Methods Mol. Cell Bio. 4:220-229), the E. coli maltose binding protein (Guanetal., (1987) Gene 67:21-30), and various cellulose binding domains (U.S. Pat. Nos. 5,496,934: 5,202.247; 5,137,819; Tomme et al., (1994) Protein Eng. 7:117-123), etc.

Peptide tags contemplated herein include short amino acid sequences to which monoclonal antibodies are available, such as but not limited to the following well-known examples, the FLAG epitope, the myc epitope at amino acids 408-439, the influenza virus hemaglutinin (HA) epitope. Other peptide tags are recognized by specific binding partners and thus facilitate isolation by affinity binding to the binding partner, which is preferably immobilized and/or on a solid support. As will be appreciated by those skilled in the art, many methods can be used to obtain the coding region of the above-mentioned peptide tags, including but not limited to, DNA cloning, DNA amplification, and synthetic methods. Some of the peptide tags and reagents for their detection and isolation are available commercially.

In certain embodiments, a combination of different peptide tags is used for the purification of the protein subunits of a complex encompassed by the present invention or for the purification of a complex. In certain embodiments, at least one subunit has at least two peptide tags, e.g., a FLAG tag and a His tag. The different tags can be fused together or can be fused in different positions to the protein subunit. In the purification procedure, the different peptide tags are used subsequently or concurrently for purification. In certain embodiments, at least two different subunits are fused to a peptide tag, wherein the peptide tags of the two subunits can be identical or different. Using different tagged subunits for the purification of the complex ensures that only complex will be purified and minimizes the amount of uncomplexed protein subunits, such as monomers or homodimers.

Various leader sequences known in the art can be used for the efficient secretion of a protein subunit of a complex encompassed by the present invention from bacterial and mammalian cells (von Heijne, (1985) J. Mol. Biol. 184:99-105). Leader peptides are selected based on the intended host cell, and may include bacterial, yeast, viral, animal, and mammalian sequences. For example, the herpes virus glycoprotein D leader peptide is suitable for use in a variety of mammalian cells. A preferred leader peptide for use in mammalian cells can be obtained from the V-J2-C region of the mouse immunoglobulin kappa chain (Bernard et al., (1981) Proc. Natl. Acad. Sci. 78:5812-5816).

DNA sequences encoding desired peptide tag or leader peptide which are known or readily available from libraries or commercial suppliers are suitable in the practice of this invention.

In certain embodiments, the protein subunits of a complex encompassed by the present invention are derived from the same species. In more specific embodiments, the protein subunits are all derived from human. In another specific embodiment, the protein subunits are all derived from a mammal.

In certain other embodiments, the protein subunits of a complex encompassed by the present invention are derived from a non-human species, such as, but not limited to, cow, pig, horse, cat, dog, rat, mouse, a primate (e.g., a chimpanzee, a monkey, such as a cynomolgous monkey). In certain embodiments, one or more subunits are derived from human and the other subunits are derived from a mammal other than a human to give rise to chimeric complexes.

Included within the scope encompassed by the present invention is an isolated modified protein complex in which the subunits, or homologs, derivatives, or fragments thereof, are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH4, acetylation, formylation, oxidation, reduction, metabolic synthesis in the presence of tunicamycin, etc. In still another embodiment, the protein sequences are modified to have a heterofunctional reagent; such heterofunctional reagents can be used to crosslink the members of the complex.

The protein complexes encompassed by the present invention can also be in a modified form. For example, an antibody selectively immunoreactive with the protein complex can be bound to the protein complex. In another example, a non-antibody modulator capable of enhancing the interaction between the interacting partners in the protein complex may be included.

The above-described protein complexes may further include any additional components, e.g., other proteins, nucleic acids, lipid molecules, monosaccharides or polysaccharides, ions, etc.

### XI. Methods of Preparing Polypeptides and Protein Complexes

The polypeptides and protein complexes encompassed by the present invention can be obtained by methods well-known in the art for protein purification and recombinant protein expression, as well as the methods described in details in the Examples. For example, the polypeptides and protein complexes encompassed by the present invention can be isolated using the TAP method described in Section 5, infra, and in WO 00/09716 and Rigaut et al. (1999) Nature Biotechnol., 17:1030-1032, which are each incorporated by reference in their entirety. Additionally, the polypeptides and protein complexes can be isolated by immunoprecipitation of subunit proteins and combining the immunoprecipitated proteins. The protein complexes can also be produced by recombinantly expressing the subunit proteins and combining the expressed proteins.

In certain embodiments, the complexes can be generated by co-expressing the subunits of the complex in a cell and subsequently purifying the complex. In certain, more specific embodiments, the cell expresses at least one subunit of the complex by recombinant DNA technology. In other embodiments, the cells normally express the subunits of the complex. In certain other embodiments, the subunits of the complex are expressed separately, wherein the subunits can be expressed using recombinant DNA technology or wherein at least one subunit is purified from a cell that normally expresses the subunit. The individual subunits of the complex are incubated in vitro under conditions conducive to the binding of the subunits of a complex encompassed by the present invention to each other to generate a complex encompassed by the present invention.

If one or more of the subunits is expressed by recombinant DNA technology, any method known to the skilled artisan can be used to produce the recombinant protein. The nucleic and amino acid sequences of the subunit proteins of the protein complexes encompassed by the present invention are provided herein, such as in Table 1, and can be obtained by any method known in the art, e.g., by PCR amplification using synthetic primers hybridizable to the 3′ and 5′ ends of each sequence, and/or by cloning from a cDNA or genomic library using an oligonucleotide specific for each nucleotide sequence.

For recombinant expression of one or more of the proteins, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals can also be supplied by the native promoter of the subunit protein gene, and/or flanking regions.

A variety of host-vector systems may be utilized to express the protein coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

In a preferred embodiment, a complex encompassed by the present invention is obtained by expressing the entire coding sequences of the subunit proteins in the same cell, either under the control of the same promoter or separate promoters. In yet another embodiment, a derivative, fragment or homologue of a subunit protein is recombinantly expressed. Preferably the derivative, fragment or homologue of the protein forms a complex with the other subunits of the complex, and more preferably forms a complex that binds to an anti-complex antibody.

Any method available in the art can be used for the insertion of DNA fragments into a vector to construct expression vectors containing a chimeric gene consisting of appropriate transcriptional/translational control signals and protein coding sequences. These methods may include in vitro recombinant DNA and synthetic techniques and in vivo recombinant techniques (genetic recombination). Expression of nucleic acid sequences encoding a subunit protein, or a derivative, fragment or homologue thereof, may be regulated by a second nucleic acid sequence so that the gene or fragment thereof is expressed in a host transformed with the recombinant DNA molecule(s). For example, expression of the proteins may be controlled by any promoter/enhancer known in the art. In a specific embodiment, the promoter is not native to the gene for the subunit protein. Promoters that may be used can be selected from among the many known in the art, and are chosen so as to be operative in the selected host cell.

In a specific embodiment, a vector is used that comprises a promoter operably linked to nucleic acid sequences encoding a subunit protein, or a fragment, derivative or homologue thereof, one or more origins of replication, and optionally, one or more selectable markers (e.g., an antibiotic resistance gene).

In another specific embodiment, an expression vector containing the coding sequence, or a portion thereof, of a subunit protein, either together or separately, is made by subcloning the gene sequences into the EcoRI restriction site of each of the three pGEX vectors (glutathione S-transferase expression vectors; Smith and Johnson (1988) Gene 7:31-40). This allows for the expression of products in the correct reading frame.

Expression vectors containing the sequences of interest can be identified by three general approaches: (a) nucleic acid hybridization, (b) presence or absence of “marker” gene function, and (c) expression of the inserted sequences. In the first approach, coding sequences can be detected by nucleic acid hybridization to probes comprising sequences homologous and complementary to the inserted sequences. In the second approach, the recombinant vector/host system can be identified and selected based upon the presence or absence of certain “marker” functions (e.g., resistance to antibiotics, occlusion body formation in baculovirus, etc.) caused by insertion of the sequences of interest in the vector. For example, if a subunit protein gene, or portion thereof, is inserted within the marker gene sequence of the vector, recombinants containing the encoded protein or portion will be identified by the absence of the marker gene function (e.g., loss of β-galactosidase activity). In the third approach, recombinant expression vectors can be identified by assaying for the subunit protein expressed by the recombinant vector. Such assays can be based, for example, on the physical or functional properties of the interacting species in in vitro assay systems, e.g., formation of a complex comprising the protein or binding to an anti-complex antibody.

Once recombinant subunit protein molecules are identified and the complexes or individual proteins isolated, several methods known in the art can be used to propagate them. Using a suitable host system and growth conditions, recombinant expression vectors can be propagated and amplified in quantity. As previously described, the expression vectors or derivatives which can be used include, but are not limited to, human or animal viruses such as vaccinia virus or adenovirus; insect viruses such as baculovirus, yeast vectors; bacteriophage vectors such as lambda phage; and plasmid and cosmid vectors.

In addition, a host cell strain may be chosen that modulates the expression of the inserted sequences, or modifies or processes the expressed proteins in the specific fashion desired. Expression from certain promoters can be elevated in the presence of certain inducers; thus expression of the genetically-engineered subunit proteins may be controlled. Furthermore, different host cells have characteristic and specific mechanisms for the translational and post-translational processing and modification (e.g., glycosylation, phosphorylation, etc.) of proteins. Appropriate cell lines or host systems can be chosen to ensure that the desired modification and processing of the foreign protein is achieved. For example, expression in a bacterial system can be used to produce an unglycosylated core protein, while expression in mammalian cells ensures“native” glycosylation of a heterologous protein. Furthermore, different vector/host expression systems may effect processing reactions to different extents.

In other specific embodiments, a subunit protein or a fragment, homologue or derivative thereof, may be expressed as fusion or chimeric protein product comprising the protein, fragment, homologue, or derivative joined via a peptide bond to a heterologous protein sequence of a different protein. Such chimeric products can be made by ligating the appropriate nucleic acid sequences encoding the desired amino acids to each other by methods known in the art, in the proper coding frame, and expressing the chimeric products in a suitable host by methods commonly known in the art. Alternatively, such a chimeric product can be made by protein synthetic techniques, e.g., by use of a peptide synthesizer. Chimeric genes comprising a portion of a subunit protein fused to any heterologous protein-encoding sequences may be constructed.

In particular, protein subunit derivatives can be made by altering their sequences by substitutions, additions or deletions that provide for functionally equivalent molecules. Due to the degeneracy of nucleotide coding sequences, other DNA sequences that encode substantially the same amino acid sequence as a subunit gene or cDNA can be used in the practice encompassed by the present invention. These include but are not limited to nucleotide sequences comprising all or portions of the subunit protein gene that are altered by the substitution of different codons that encode a functionally equivalent amino acid residue within the sequence, thus producing a silent change. Likewise, the derivatives encompassed by the present invention include, but are not limited to, those containing, as a primary amino acid sequence, all or part of the amino acid sequence of a subunit protein, including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

In a specific embodiment, up to 1%, 2%, 5%, 10%, 15% or 20% of the total number of amino acids in the wild-type protein are substituted or deleted; or 1, 2, 3, 4, 5, or 6 or up to 10 or up to 20 amino acids are inserted, substituted or deleted relative to the wild-type protein.

The protein subunit derivatives and analogs encompassed by the present invention can be produced by various methods known in the art. The manipulations which result in their production can occur at the gene or protein level. For example, the cloned gene sequences can be modified by any of numerous strategies known in the art (Sambrook et al. (1989) Molecular Cloning, A Laboratory Manual, 2d Ed., Cold Spring Harbor Laboratory Press, Cold Spring Harbor, N.Y.). The sequences can be cleaved at appropriate sites with restriction endonuclease(s), followed by further enzymatic modification if desired, isolated, and ligated in vitro. In the production of the gene encoding a derivative, homologue or analog of a subunit protein, care should be taken to ensure that the modified gene retains the original translational reading frame, uninterrupted by translational stop signals, in the gene region where the desired activity is encoded.

Additionally, the encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, chemical mutagenesis and in vitro site-directed mutagenesis (Hutchinson et al. (1978) J. Bioi. Chern. 253:6551-6558), amplification with PCR primers containing a mutation, etc.

Once a recombinant cell expressing a subunit protein, or fragment or derivative thereof, is identified, the individual gene product or complex can be isolated and analyzed. This is achieved by assays based on the physical and/or functional properties of the protein or complex, including, but not limited to, radioactive labeling of the product followed by analysis by gel electrophoresis, immunoassay, cross-linking to marker-labeled product, etc.

The subunit proteins and complexes may be isolated and purified by standard methods known in the art (either from natural sources or recombinant host cells expressing the complexes or proteins) or methods described in the examples herein, including but not restricted to column chromatography (e.g., ion exchange, affinity, gel exclusion, reversed-phase high pressure, fast protein liquid, etc.), differential centrifugation, differential solubility, or by any other standard technique used for the purification of proteins. In some embodiment, the isolation methods include the density sedimentation-based approaches. Functional properties may be evaluated using any suitable assay known in the art.

Alternatively, once a subunit protein or its derivative, is identified, the amino acid sequence of the protein can be deduced from the nucleic acid sequence of the chimeric gene from which it was encoded. As a result, the protein or its derivative can be synthesized by standard chemical methods known in the art (e.g., Hunkapiller et al. (1984) Nature 310:105-111).

In addition, complexes of analogs and derivatives of subunit proteins can be chemically synthesized. For example, a peptide corresponding to a portion of a subunit protein, which comprises the desired domain or mediates the desired activity in vitro (e.g., complex formation) can be synthesized by use of a peptide synthesizer.

Furthermore, if desired, non-classical amino acids or chemical amino acid analogs can be introduced as a substitution or addition into the protein sequence. Non-classical amino acids include but are not limited to the D-isomers of the common amino acids, α-amino isobutyric acid, 4-aminobutyric acid (4-Abu), 2-aminobutyric acid (2-Abu), 6-amino hexanoic acid (Ahk), 2-amino isobutyric acid (2-Aib), 3-amino propionoic acid, ornithine, norleucine, norvaline, hydroxyproline, sarcosine, citrulline, cysteic acid. t-butylglycine, t-butylalanine, phenylglycine, cyclohexylalanine, β-alanine, fluoro-amino acids, designer amino acids such as β-methyl amino acids, Ca-methyl amino acids. Na-methylamino acids, and amino acid analogs in general. Furthermore, the amino acid can be D (dextrorotary) or L (levorotary).

In cases where natural products are suspected of being mutant or are purified from new species, the amino acid sequence of a subunit protein purified from the natural Source. as well as those expressed in vitro, or from synthesized expression vectors in vivo or in vitro, can be determined from analysis of the DNA sequence, or alternatively, by direct sequencing of the purified protein. Such analysis can be performed by manual sequencing or through use of an automated amino acid sequenator.

The complexes can also be analyzed by hydrophilicity analysis (Hopp and Woods (1981) Proc. Natl. Acad. Sci. USA 78:3824-3828). A hydrophilicity profile can be used to identify the hydrophobic and hydrophilic regions of the proteins, and help predict their orientation in designing substrates for experimental manipulation, such as in binding experiments, antibody synthesis, etc. Secondary structural analysis can also be done to identify regions of the subunit proteins, or their derivatives, that assume specific structures (Chou and Fasman (1974) Biochemistry 13:222-23). Manipulation, translation, secondary structure prediction, hydrophilicity and hydrophobicity profile predictions, open reading frame prediction and plotting, and determination of sequence homologies, etc., can be accomplished using computer software programs available in the art.

Other methods of structural analysis including but not limited to X-ray crystallography (Engstrom (1974) Biochem. Exp. Biol. 11:7-13), mass spectroscopy and gas chromatography (Methods in Protein Science, J. Wiley and Sons, New York, 1997), and computer modeling (Fietterick and Zoller eds. (1986) Computer Graphics and Molecular Modeling, In Current Communications in Molecular Biology, Cold Spring Harbor Laboratory, Cold Spring Harbor Press, New York) can also be employed.

In certain embodiments, at least one subunit of the complex is generated by recombinant DNA technology and is a derivative of the naturally occurring protein. In certain embodiments, the derivative is a fusion protein, wherein the amino acid sequence of the naturally occurring protein is fused to a second amino acid sequence. The second amino acid sequence can be a peptide tag that facilitates the purification, immunological detection and identification as well as visualization of the protein. A variety of peptide tags with different functions and affinities can be used in the invention to facilitate the purification of the subunit or the complex comprising the subunit by affinity chromatography. A specific peptide tag comprises the constant regions of an immunoglobulin. In other embodiments, the subunit is fused to a leader sequence to promote secretion of the protein subunit from the cell that expresses the protein subunit. Other peptide tags that can be used with the invention include, but are not limited to, FLAG epitope or HA tag.

If the subunits of the complex are co-expressed, the complex can be purified by any method known to the skilled artisan, including immunoprecipitation, ammonium Sulfate precipitation, acid extraction, anion or cation exchange chromatography, phosphocellulose chromatography, immunoaffinity chromatography, hydroxyapatite chromatography, and lectin chromatography.

The methods described herein can be used to purify the individual subunits of the complex encompassed by the present invention. The methods can also be used to purify the entire complex. Generally, the purification conditions as well as the dissociation constant of the complex will determine whether the complex remains intact during the purification procedure. Such conditions include, but are not limited to, salt concentration, detergent concentration, pH and redox-potential.

If at least one polypeptide, or subunit of the complex, comprises a peptide tag, the invention also contemplates methods for the purification of the complexes encompassed by the present invention which are based on the properties of the peptide tag. One approach is based on specific molecular interactions between a tag and its binding partner. The other approach relies on the immunospecific binding of an antibody to an epitope present on the tag. The principle of affinity chromatography well-known in the art is generally applicable to both of these approaches. In another embodiment, the complex is purified using immunoprecipitation.

In certain embodiments, the individual subunits of a complex encompassed by the present invention are expressed separately. The subunits are subsequently incubated under conditions conducive to the binding of the subunits of the complex to each other to generate the complex. In certain, more specific embodiments, the subunits are purified before complex formation. In other embodiments the supernatants of cells that express the subunit (if the subunit is secreted) or cell lysates of cells that express the subunit (if the subunit is not secreted) are combined first to give rise to the complex, and the complex is purified subsequently. Parameters affecting the ability of the subunits encompassed by the present invention to bind to each other include, but are not limited to, salt concentration, detergent concentration, pH, and redox-potential. Once the complex has formed, the complex can be purified or concentrated by any method known to the skilled artisan. In certain embodiments, the complex is separated from the remaining individual subunits by filtration. The pore size of the filter should be such that the individual subunits can still pass through the filter, but the complex does not pass through the filter. Other methods for enriching the complex include sucrose gradient centrifugation and chromatography.

### XII. Screening Methods

a. Modulators of Complex Formation

A complex encompassed by the present invention, the component proteins of the complex and nucleic acids encoding the component proteins, as well as derivatives and fragments of the amino and nucleic acids, can be used to screen for compounds that bind to, or modulate the amount of, activity of, formation of, or stability of, said complex, and thus, have potential use as modulators, i.e., agonists or antagonists, of complex activity, complex stability, and/or complex formation, i.e., the amount of complex formed, and/or protein component composition of the complex.

As described above, complexes for use according to the present invention can be single polypeptides in association with another moiety or combinations of polypeptides (e.g., protein complexes) in association with each other and/or in association with another moiety.

Thus, present invention is also directed to methods for screening for molecules that bind to, or modulate the amount of activity of protein component composition of a complex encompassed by the present invention. In one embodiment encompassed by the present invention, the method for screening for a molecule that modulates directly or indirectly the function, activity or formation of a complex encompassed by the present invention comprises exposing said complex, or a cell or organism containing the complex machinery, to one or more test agents under conditions conducive to modulation; and determining the amount of activity of or identities of the protein components of said complex, wherein a change in said amount, activity, or identities relative to said amount, activity or identities in the absence of the test agents indicates that the test agents modulate function, activity or formation of said complex. Such screening assays can be carried out using cell-free and cell-based methods that are commonly known in the art.

In one embodiment, the method for screening for molecules that bind to, or modulate the amount of, activity of, formation of, or stability of, a complex encompassed by the present invention further comprises incubating subunits of the isolated modified protein complex in the presence of a test agent under conditions conductive to form the modified protein complex prior to step of contacting described above. In another embodiment, the method further comprises a step of determining the presence and/or amount of the individual subunits in the isolated modified protein complex.

The present invention is further directed to methods for screening for molecules that modulate the expression of a subunit of a complex encompassed by the present invention. In one embodiment encompassed by the present invention, the method for screening for a molecule that modulates the expression of a subunit of a complex encompassed by the present invention comprises exposing a cell or organism containing the nucleic acid encoding the component, to one or more compounds under conditions conducive to modulation; and determining the amount of activity of, or identities of the protein components of said complex, wherein a change in said amount, activity, or identities relative to said amount, activity or identities in the absence of said compounds indicates that the compounds modulate expression of said complex. Such screening assays can be carried out using cell-free and cell based methods that are commonly known in the art. If activity of the complex or component is used as read-out of the assay, subsequent assays, such as western blot analysis or northern blot analysis, may be performed to verify that the modulated expression levels of the component are responsible for the modulated activity.

In a further specific embodiment, a modulation of the formation or stability of a complex can be determined. In some embodiment, the agent modulates (inhibits or promotes) the formation or stability of the isolated modified protein complex. In specific embodiments, the agent inhibits the formation or stability of the isolated modified protein complex by inhibiting or promoting the interaction between at least one interaction between a polypeptide in the complex and another subunit listed in Tables 1-5. The agent may be, e.g., a small molecule inhibitor, a small molecule degrader, CRISPR guide RNA (gRNA), RNA interfering agent, oligonucleotide, peptide or peptidomimetic inhibitor, aptamer, antibody, or intrabody. In a specific embodiment, the agent comprises an antibody and/or intrabody, or an antigen binding fragment thereof, which specifically binds to at least one subunit of the isolated modified protein complex. In some other embodiments, the agent enhances the formation or stability of the isolated modified protein complex. In specific embodiments, the agent enhances the formation or stability of the protein complex by stabilizing the interaction between at least one interaction between a polypeptide of the complex and another subunit listed in Tables 1-5. The agent may be a small molecule compound, e.g., a small molecule stabilizer.

Such a modulation can either be a change in the typical time course of its formation or a change in the typical steps leading to the formation of the complete complex. Such changes can for example be detected by analyzing and comparing the process of complex formation in untreated wild-type cells of a particular type and/or cells showing or having the predisposition to develop a certain disease phenotype and/or cells that have been treated with particular conditions and/or particular agents in a particular situation. Methods to study such changes in time course are well-known in the art and include for example Western blot analysis of the proteins in the complex isolated at different steps of its formation.

In a specific embodiment, fragments and/or analogs of protein components of a complex, especially peptidomimetics, are screened for activity as competitive or non-competitive inhibitors of complex formation, which thereby inhibit complex activity or formation.

In another embodiment, the present invention is directed to a method for screening for a molecule that binds a protein complex encompassed by the present invention comprising exposing said complex, or a cell or organism containing the complex machinery, to one or more candidate molecules; and determining whether said complex is bound by any of said candidate molecules.

Screening the libraries can be accomplished by any of a variety of commonly known methods. See, e.g., the following references, which disclose screening of peptide libraries: Parmley and Smith (1989) Adv. Exp. Med. Biol. 251:215-218: Scott and Smith (1990) Science 249:386-390; Fowlkes et al. (1992) BioTechniques 13:422-427; Oldenburg et al. (1992) Proc. Natl. Acad. Sci. USA 89:5393-5397: Yu et al. (1994) Cell 76:933-945; Staudt et al. (1988) Science 241: 577-580; Bock et al. (1992) Nature 355:564-566: Tuerk et al. (1992) Proc. Natl. Acad. Sci. USA 89:6988-6992: Ellington et al. (1992) Nature 355:850-852; U.S. Pat. Nos. 5,096,815, 5,223,409, and 5,198,346, all to Ladner et al. Rebar and Pabo, (1993) Science 263:671-673; and International Patent Publication No. WO 94/18318.

In a specific embodiment, screening can be carried out by contacting the library members with a complex immobilized on a solid phase, and harvesting those library members that bind to the protein (or encoding nucleic acid or derivative). Examples of such screening methods, termed “panning” techniques, are described by way of example in Parmley and Smith (1988), Gene 73:305-318; Fowlkes et al. (1992), BioTechniques 13:422-427; International Patent Publication No. WO 94/18318; and in references cited herein above.

In a specific embodiment, fragments and/or analogs of protein components of a complex, especially peptidomimetics, are screened for activity as competitive or non-competitive inhibitors of complex formation (amount of complex or composition of complex) or activity in the cell, which thereby inhibit complex activity or formation in the cell.

In one embodiment, agents that modulate (i.e., antagonize or agonize) complex activity or formation can be screened for using a binding inhibition assay, wherein agents are screened for their ability to modulate formation of a complex under aqueous, or physiological, binding conditions in which complex formation occurs in the absence of the agent to be tested. Agents that interfere with the formation of complexes encompassed by the present invention are identified as antagonists of complex formation. Agents that promote the formation of complexes are identified as agonists of complex formation.

Agents that completely block the formation of complexes are identified as inhibitors of complex formation.

Methods for screening may involve labeling the component proteins of the complex with radioligands (e.g., ¹²⁵1 or ³H), magnetic ligands (e.g., paramagnetic beads covalently attached to photobiotin acetate), fluorescent ligands (e.g., fluorescein or rhodamine), or enzyme ligands (e.g., luciferase or p-galactosidase). The reactants that bind in solution can then be isolated by one of many techniques known in the art, including but not restricted to, co-immunoprecipitation of the labeled complex moiety using antisera against the unlabeled binding partner (or labeled binding partner with a distinguishable marker from that used on the second labeled complex moiety), immunoaffinity chromatography, size exclusion chromatography, and gradient density centrifugation. In a preferred embodiment, the labeled binding partner is a small fragment or peptidomimetic that is not retained by a commercially available filter. Upon binding, the labeled species is then unable to pass through the filter, providing for a simple assay of complex formation.

In certain embodiments, the protein components of a complex encompassed by the present invention are labeled with different fluorophores such that binding of the components to each other results in FRET (Fluorescence Resonance Energy Transfer). If the addition of a compound results in a difference in FRET compared to FRET in the absence of the compound, the compound is identified as a modulator of complex formation. If FRET in the presence of the compound is decreased in comparison to FRET in the absence of the compound, the compound is identified as an inhibitor of complex formation. If FRET in the presence of the compound is increased in comparison to FRET in the absence of the compound, the compound is identified as an activator of complex formation.

In certain other embodiments, a protein component of a complex encompassed by the present invention is labeled with a fluorophore such that binding of the component to another protein component to form a complex encompassed by the present invention results in FP (Fluorescence Polarization). If the addition of a compound results in a difference in FP compared to FP in the absence of the compound, the compound is identified as a modulator of complex formation.

Methods commonly known in the art are used to label at least one of the component members of the complex. Suitable labeling methods include, but are not limited to, radiolabeling by incorporation of radiolabeled amino acids, e.g., ³H-Ieucine or ³⁵8-methionine, radiolabeling by post-translational iodination with ¹²⁵I or ¹³¹I using the chloramine T method, Bolton-Hunter reagents, etc., or labeling with ³²P using phosphorylase and inorganic radiolabeled phosphorous, biotin labeling with photobiotin-acetate and sunlamp exposure, etc. In cases where one of the members of the complex is immobilized, e.g., as described infra, the free species is labeled. Where neither of the interacting species is immobilized, each can be labeled with a distinguishable marker such that isolation of both moieties can be followed to provide for more accurate quantification, and to distinguish the formation of homomeric from heteromeric complexes. Methods that utilize accessory proteins that bind to one of the modified interactants to improve the sensitivity of detection, increase the stability of the complex, etc., are provided.

The physical parameters of complex formation can be analyzed by quantification of complex formation using assay methods specific for the label used, e.g., liquid scintillation counting for radioactivity detection, enzyme activity for enzyme-labeled moieties, etc. The reaction results are then analyzed utilizing Scatchard analysis, Hill analysis, and other methods commonly known in the arts (see, e.g., Proteins, Structures, and Molecular Principles, 2nd Edition (1993) Creighton, Ed., W.H. Freeman and Company, New York).

Agents/molecules (candidate molecules) to be screened can be provided as mixtures of a limited number of specified compounds, or as compound libraries, peptide libraries and the like. Agents/molecules to be screened may also include all forms of antisera, antisense nucleic acids, etc., that can modulate complex activity or formation. Exemplary candidate molecules and libraries for screening are set forth below.

In certain embodiments, the compounds are screened in pools. Once a positive pool has been identified, the individual molecules of that pool are tested separately. In certain embodiments, the pool size is at least 2, at least 5, at least 10, at least 25, at least 50, at least 75, at least 100, at least 150, at least 200, at least 250, or at least 500 compounds.

In certain embodiments encompassed by the present invention, the screening method further comprises determining the structure of the candidate molecule. The structure of a candidate molecule can be determined by any technique known to the skilled artisan.

i. Test Agents

Any molecule known in the art can be tested for its ability to modulate (increase or decrease) the amount of, activity of, or protein component composition of a complex encompassed by the present invention as detected by a change in the amount of, activity of, or protein component composition of said complex. By way of example, a change in the amount of the complex can be detected by detecting a change in the amount of the complex that can be isolated from a cell expressing the complex machinery. In other embodiments, a change in signal intensity (e.g., when using FRET or FP) in the presence of a compound compared to the absence of the compound indicates that the compound is a modulator of complex formation. For identifying a molecule that modulates complex activity, candidate molecules can be directly provided to a cell expressing the complex, or, in the case of candidate proteins, can be provided by providing their encoding nucleic acids under conditions in which the nucleic acids are recombinantly expressed to produce the candidate proteins within the cell expressing the complex machinery, the complex is then purified from the cell and the purified complex is assayed for activity using methods well-known in the art, not limited to those described, supra.

In certain embodiments, the invention provides screening assays using chemical libraries for molecules which modulate, e.g., inhibit, antagonize, or agonize, the amount of, activity of, or protein component composition of the complex. The chemical libraries can be peptide libraries, peptidomimetic libraries, chemically synthesized libraries, recombinant, e.g., phage display libraries, and in vitro translation-based libraries, other non-peptide synthetic organic libraries, etc.

Exemplary libraries are commercially available from several sources (ArOule, Tripos/PanLabs, ChemDesign, and Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 chemicals that can be screened by administration to a well containing the interactions of interest. Thus, if modulation is detected, smaller and smaller pools of interacting pairs can be assayed for the modulation activity. By such methods, many candidate molecules can be screened.

Many diverse libraries suitable for use are known in the art and can be used to provide compounds to be tested according to the present invention. Alternatively, libraries can be constructed using standard methods. Chemical (synthetic) libraries, recombinant expression libraries, or polysome based libraries are exemplary types of libraries that can be used.

The libraries can be constrained or semirigid (having some degree of structural rigidity), or linear or non-constrained. The library can be a cDNA or genomic expression library, random peptide expression library or a chemically synthesized random peptide library, or non-peptide library. Expression libraries are introduced into the cells in which the assay occurs, where the nucleic acids of the library are expressed to produce their encoded proteins.

In one embodiment, peptide libraries that can be used in the present invention may be libraries that are chemically synthesized in vitro. Examples of such libraries are given in Houghten et al. (1991) Nature 354:84-86, which describes mixtures of free hexapeptides in which the first and second residues in each peptide were individually and specifically defined; Lam et al. (1991) Nature 354:82-84, which describes a “one bead, one peptide’ approach in which a solid phase split synthesis scheme produced a library of peptides in which each bead in the collection had immobilized thereon a single, random sequence of amino acid residues; Medynski (1994) Bio/Technology 12:709-710, which describes split synthesis and T-bag synthesis methods; and Gallop et al. (1994) J. Medicinal Chemistry 37(9): 1233-1251. Simply by way of other examples, a combinatorial library may be prepared for use, according to the methods of Ohlmeyer et al. (1993) Proc. Natl. Acad. Sci. USA 90:10922-10926; Erb et al. (1994) Proc. Natl. Acad Sci. USA 91:11422-11426; Houghten et al., (1992) Biotechniques 13:412; Jayawickreme et al. (1994), Proc. Natl. Acad Sci. USA 91:1614-1618; or Salmon et al. (1993) Proc. Natl. Acad Sci. USA 90:11708-11712. PCT Publication No. WO 93/20242 and Brenner and Lerner (1992), Proc. Natl. Acad Sci. USA 89:5381-5383 describe “encoded combinatorial chemical libraries,” that contain oligonucleotide identifiers for each chemical polymer library member.

In a preferred embodiment, the library screened is a biological expression library that is a random peptide phage display library, where the random peptides are constrained (e.g., by virtue of having disulfide bonding).

Further, more general, structurally constrained, organic diversity (e.g., nonpeptide) libraries, can also be used.

Conformationally constrained libraries that can be used include but are not limited to those containing invariant cysteine residues which, in an oxidizing environment, cross link by disulfide bonds to form cystines, modified peptides (e.g., incorporating fluorine, metals, isotopic labels, are phosphorylated, etc.), peptides containing one or more non-naturally occurring amino acids, non-peptide structures, and peptides containing a significant fraction of Y-carboxyglutamic acid.

Libraries of non-peptides, e.g., peptide derivatives (for example that contain one or more non-naturally occurring amino acids) can also be used. One example of these are peptoid libraries (Simon et al. (1992) Proc. Natl. Acad. Sci. USA 89:9367-9371). Peptoids are polymers of non-natural amino acids that have naturally occurring side chains attached not to the alpha carbon but to the backbone amino nitrogen.

Since peptoids are not easily degraded by human digestive enzymes, they are advantageously more easily adaptable to drug use. Another example of a library that can be used, in which the amide functionalities in peptides have been permethylated to generate a chemically transformed combinatorial library, is described by Ostresh et al. (1994) Proc. Natl. Acad. Sci. USA 91:11138-11142).

The members of the peptide libraries that can be screened according to the invention are not limited to containing the 20 naturally occurring amino acids. In particular, chemically synthesized libraries and polysome based libraries allow the use of amino acids in addition to the 20 naturally occurring amino acids (by their inclusion in the precursor pool of amino acids used in library production). In specific embodiments, the library members contain one or more non-natural or non-classical amino acids or cyclic peptides. Non-classical amino acids include but are not limited to the D-isomers of the common amino acids, α-amino isobutyric acid, 4-aminobutyric acid, Abu, 2-amino butyric acid; γ-Abu, ε-Ahk, 6-amino hexanoic acid; Aib, 2-amino isobutyric acid: 3-amino propionic acid: ornithine; norleucine: norvaline, hydroxyproline, sarcosine, citrulline, cysteic acid, t-butylglycine, t-butylalanine, phenylglycine, cyclohexylalanine, β-alanine, designer amino acids such as β-methyl amino acids, Ca-methyl amino acids, Na-methyl amino acids, fluoro-amino acids and amino acid analogs in general. Furthermore, the amino acid can be D (dextrorotary) or L (levorotary).

In a specific embodiment, fragments and/or analogs of protein components of complexes encompassed by the present invention, especially peptidomimetics, are screened for activity as competitive or non-competitive inhibitors of complex activity or formation.

In another embodiment encompassed by the present invention, combinatorial chemistry can be used to identify modulators of the complexes. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See, e.g., Matter (1997) Journal of Medicinal Chemistry 40:1219-1229).

One method of combinatorial chemistry, affinity fingerprinting, has previously been used to test a discrete library of small molecules for binding affinities for a defined panel of proteins. The fingerprints obtained by the Screen are used to predict the affinity of the individual library members for other proteins or receptors of interest (in the instant invention, the protein complexes encompassed by the present invention and protein components thereof) The fingerprints are compared with fingerprints obtained from other compounds known to react with the protein of interest to predict whether the library compound might similarly react. For example, rather than testing every ligand in a large library for interaction with a complex or protein component, only those ligands having a fingerprint similar to other compounds known to have that activity could be tested. (See, e.g., Kauvar et al. (1995) Chemistry and Biology 2:107-118; Kauvar (1995) Affinity finger printing, Pharmaceutical Manufacturing International. 8:25-28; and Kauvar, Toxic-Chemical Detection by Pattern Recognition in New Frontiers in Agrochemical Immunoassay, D. Kurtz. L. Stanker and J. H. Skerritt. Editors, 1995, AOAC: Washington, D.C., 305-312).

Kay et al. (1993) Gene 128:59-65 (Kay) discloses a method of constructing peptide libraries that encode peptides of totally random sequence that are longer than those of any prior conventional libraries. The libraries disclosed in Kay encode totally synthetic random peptides of greater than about 20 amino acids in length. Such libraries can be advantageously screened to identify complex modulators. (See also U.S. Pat. No. 5,498,538 dated Mar. 12, 1996; and PCT Publication No. WO 94/18318 dated Aug. 18, 1994).

A comprehensive review of various types of peptide libraries can be found in Gallop et al. (1994) J. Med. Chem. 37:1233-1251.

Libraries screened using the methods encompassed by the present invention can comprise a variety of types of compounds. Examples of libraries that can be screened in accordance with the methods encompassed by the present invention include, but are not limited to, peptoids; random biooligomers; diversomers such as hydantoins, benzodiazepines and dipeptides; vinylogous polypeptides; nonpeptidal peptidomimetics; oligocarbamates; peptidyl phosphonates; peptide nucleic acid libraries; antibody libraries; carbohydrate libraries; and small molecule libraries (preferably, small organic molecule libraries). In some embodiments, the compounds in the libraries screened are nucleic acid or peptide molecules. In a non-limiting example, peptide molecules can exist in a phage display library. In other embodiments, the types of compounds include, but are not limited to, peptide analogs including peptides comprising non-naturally occurring amino acids, e.g., D-amino acids, phosphorous analogs of amino acids, such as α-amino phosphoric acids and α-amino phosphoric acids, or amino acids having non-peptide linkages, nucleic acid analogs such as phosphorothioates and PNAs, hormones, antigens, synthetic or naturally occurring drugs, opiates, dopamine, serotonin, catecholamines, thrombin, acetylcholine, prostaglandins, organic molecules, pheromones, adenosine, sucrose, glucose, lactose and galactose. Libraries of polypeptides or proteins can also be used in the assays encompassed by the present invention.

In a preferred embodiment, the combinatorial libraries are small organic molecule libraries including, but not limited to, benzodiazepines, isoprenoids, thiazolidinones, metathiazanones, pyrrolidines, morpholino compounds, and benzodiazepines. In another embodiment, the combinatorial libraries comprise peptoids; random bio-oligomers; benzodiazepines; diversomers such as hydantoins, benzodiazepines and dipeptides; vinylogous polypeptides; nonpeptidal peptidomimetics; oligocarbamates; peptidyl phosphonates; peptide nucleic acid libraries; antibody libraries; or carbohydrate libraries. Combinatorial libraries are themselves commercially available (see, e.g., ComGenex, Princeton, N.J.; Asinex, Moscow, Ru, Tripos, Inc., St. Louis, Mo.; ChemStar, Ltd, Moscow, Russia; 3D Pharmaceuticals, Exton, Pa.; Martek Biosciences, Columbia, Md.; etc.).

In a preferred embodiment, the library is preselected so that the compounds of the library are more amenable for cellular uptake. For example, compounds are selected based on specific parameters such as, but not limited to, size, lipophilicity, hydrophilicity, and hydrogen bonding, which enhance the likelihood of compounds getting into the cells. In another embodiment, the compounds are analyzed by three-dimensional or four-dimensional computer computation programs.

The combinatorial compound library for use in accordance with the methods encompassed by the present invention may be synthesized. There is a great interest in synthetic methods directed toward the creation of large collections of small organic compounds, or libraries, which could be screened for pharmacological, biological or other activity. The synthetic methods applied to create vast combinatorial libraries are performed in solution or in the solid phase, i.e., on a solid support. Solid-phase synthesis makes it easier to conduct multi-step reactions and to drive reactions to completion with high yields because excess reagents can be easily added and washed away after each reaction step. Solid-phase combinatorial synthesis also tends to improve isolation, purification and screening. However, the more traditional solution phase chemistry supports a wider variety of organic reactions than solid-phase chemistry.

Combinatorial compound libraries encompassed by the present invention may be synthesized using the apparatus described in U.S. Pat. No. 6,190,619 to Kilcoin et al., which is hereby incorporated by reference in its entirety. U.S. Pat. No. 6,190,619 discloses a synthesis apparatus capable of holding a plurality of reaction vessels for parallel synthesis of multiple discrete compounds or for combinatorial libraries of compounds.

In one embodiment, the combinatorial compound library can be synthesized in solution. The method disclosed in U.S. Pat. No. 6,194,612 to Boger et al., which is hereby incorporated by reference in its entirety, features compounds useful as templates for solution phase synthesis of combinatorial libraries.

The template is designed to permit reaction products to be easily purified from unreacted reactants using liquid/liquid or solid/liquid extractions. The compounds produced by combinatorial synthesis using the template will preferably be small organic molecules. Some compounds in the library may mimic the effects of non-peptides or peptides.

In contrast to solid phase synthesize of combinatorial compound libraries, liquid phase synthesis does not require the use of specialized protocols for monitoring the individual steps of a multistep solid phase synthesis (Egner et al. (1995) J. Org. Chem. 60:2652; Anderson et al. (1995) J. Org. Chem. 60:2650; Fitch et al. (1994) J. Org. Chem. 59:7955; Look et al. (1994) J. Org. Chem. 49:7588; Metzger et al. (1993) Angew. Chem., Int. Ed. Engl. 32:894; Youngquist et al. (1994) Rapid Commun. Mass Spect. 8:77; Chu et al. (1995) J. Am. Chern. Soc. 117:5419; Brummel et al. (1994) Science 264:399; and Stevanovic et al. (1993) Bioorg. Med. Chern. Lett. 3:431).

Combinatorial compound libraries useful for the methods encompassed by the present invention can be synthesized on solid supports. In one embodiment, a split synthesis method, a protocol of separating and mixing solid supports during the synthesis, is used to synthesize a library of compounds on solid supports (see e.g., Lam et al. (1997) Chem. Rev. 97:41-448; Ohlmeyer et al. (1993) Proc. Nat. Acad. Sci. USA 90:10922-10926 and references cited therein). Each solid support in the final library has substantially one type of compound attached to its surface. Other methods for synthesizing combinatorial libraries on solid supports, wherein one product is attached to each support, will be known to those of skill in the art (see, e.g., Nefzi et al. (1997) Chem. Rev. 97:449-472).

As used herein, the term “solid support” is not limited to a specific type of solid support. Rather a large number of supports are available and are known to one skilled in the art. Solid supports include silica gels, resins, derivatized plastic films, glass beads, cotton, plastic beads, polystyrene beads, alumina gels, and polysaccharides. A suitable solid support may be selected on the basis of desired end use and suitability for various synthetic protocols. For example, for peptide synthesis, a solid support can be a resin such as p-methylbenzhydrylamine (pMBHA) resin (Peptides International, Louisville, Ky.), polystyrenes (e.g., PAM-resin obtained from Bachem Inc., Peninsula Laboratories, etc.), including chloromethylpolystyrene, hydroxymethylpolystyrene and aminomethylpolystyrene, poly (dimethylacrylamide)-grafted styrene co-divinyl-benzene (e.g., POLYHIPE resin, obtained from Aminotech, Canada), polyamide resin (obtained from Peninsula Laboratories), polystyrene resin grafted with polyethylene glycol (e.g., TENTAGEL or ARGOGEL, Bayer, Tubingen, Germany) polydimethylacrylamide resin (obtained from Milligen/Biosearch, California), or Sepharose (Pharmacia, Sweden).

In some embodiments encompassed by the present invention, compounds can be attached to solid supports via linkers. Linkers can be integral and part of the solid support, or they may be nonintegral that are either synthesized on the solid support or attached thereto after synthesis. Linkers are useful not only for providing points of compound attachment to the solid support, but also for allowing different groups of molecules to be cleaved from the solid support under different conditions, depending on the nature of the linker. For example, linkers can be, inter alia, electrophilically cleaved, nucleophilically cleaved, photocleavable, enzymatically cleaved, cleaved by metals, cleaved under reductive conditions or cleaved under oxidative conditions. In a preferred embodiment, the compounds are cleaved from the solid support prior to high throughput screening of the compounds.

In certain embodiments encompassed by the present invention, the agent is a small molecule.

ii. Cell-Free Assays

In certain embodiments, the method for identifying a modulator of the formation or stability of a complex encompassed by the present invention can be carried out in vitro, particularly in a cell-free system. In certain, more specific embodiments, the complex is purified. In certain embodiments the candidate molecule is purified.

In a specific embodiment, screening can be carried out by contacting the library members with a complex immobilized on a solid phase, and harvesting those library members that bind to the protein (or encoding nucleic acid or derivative). Examples of such screening methods, termed “panning techniques, are described by way of example in Parmley and Smith (1988) Gene 73:305-318: Fowlkes et al. (1992) BioTechniques 13:422-427: International Patent Publication No. WO 94/18318; and in references cited herein above.

In one embodiment, agents that modulate (i.e., antagonize or agonize) complex activity or formation can be screened for using a binding inhibition assay, wherein agents are screened for their ability to modulate formation of a complex under aqueous, or physiological, binding conditions in which complex formation occurs in the absence of the agent to be tested. Agents that interfere with the formation of complexes encompassed by the present invention are identified as antagonists of complex formation. Agents that promote the formation of complexes are identified as agonists of complex formation. Agents that completely block the formation of complexes are identified as inhibitors of complex formation. In an exemplary embodiment, the binding conditions are, for example, but not by way of limitation, in an aqueous salt solution of 10-250 mM NaCl, 5-50 mM Tris-HCl, pH 5-8, and 0.5% Triton X-100 or other detergent that improves specificity of interaction. Metal chelators and/or divalent cations may be added to improve binding and/or reduce proteolysis. Reaction temperatures may include 4, 10, 15, 22, 25, 35, or 42 degrees Celsius, and time of incubation is typically at least 15 seconds, but longer times are preferred to allow binding equilibrium to occur. Particular complexes can be assayed using routine protein binding assays to determine optimal binding conditions for reproducible binding.

Determining the interaction between two molecules can be accomplished using standard binding or enzymatic analysis assays. These assays may include thermal shift assays (measure of variation of the melting temperature of the protein alone and in the presence of a molecule) (R. Zhang, F. Monsma, (2010) Curr. Opin. Drug Discov. Devel., 13:389-402), SPR (surface plasmon resonance) (T. Neumann et al. (2007), Curr. Top Med. Chem., 7: 1630-1642), FRET/BRET (Fluorescence or Bioluminescence Resonance Excitation Transfer) (A. L. Mattheyses, A. I. Marcus, (2015), Methods Mol. Biol., 1278:329-339; J. Bacart, et al. (2008), Biotechnol. J., 3: 311-324), Elisa (Enzyme-linked immunosorbent assay) (Z. Weng, Q. Zhao, (2015), Methods Mol. Biol., 1278:341-352), fluorescence polarization (Y. Du, (2015), Methods Mol. Biol., 1278: 529-544), and Far western (U. Mahlknecht, O. G. Ottmann, D. Hoelzer J. (2001), Biotechnol., 88: 89-94) or other techniques. More sophisticated (and lower throughput) biophysical methods that provide structural or thermodynamic details of the molecule binding mode (using isothermal calorimetry (ITC), Nuclear Magnetic Resonance (NMR), and X-ray crystallography) may also be needed for further validation and characterization of potential hits.

For example, in a direct binding assay, one subunit (or their respective binding partners) can be coupled with a radioisotope or enzymatic label such that binding can be determined by detecting the labeled subunit in a complex. For example, the subunits can be labeled with ¹²⁵I, ³⁵S, ¹⁴C, or ³H, either directly or indirectly, and the radioisotope detected by direct counting of radioemmission or by scintillation counting. Alternatively, the subunits can be enzymatically labeled with, for example, horseradish peroxidase, alkaline phosphatase, or luciferase, and the enzymatic label detected by determination of conversion of an appropriate substrate to product.

In certain embodiments, another common approach to in vitro binding assays is used. In this assay, one of the binding species is immobilized on a filter, in a microtiter plate well, in a test tube, to a chromatography matrix, etc., either covalently or non-covalently. Proteins can be covalently immobilized using any method well-known in the art, for example, but not limited to the method of Kadonaga and Tjian (1986) Proc. Natl. Acad. Sci. USA 83:5889-5893, i.e., linkage to a cyanogen-bromide derivatized substrate such as CNBr-Sepharose 48 (Pharmacia). Where needed, the use of spacers can reduce steric hindrance by the substrate. Non-covalent attachment of proteins to a substrate include, but are not limited to, attachment of a protein to a charged surface, binding with specific antibodies, binding to a third unrelated interacting protein, etc.

Assays of agents (including cell extracts or a library pool) for competition for binding of one member of a complex (or derivatives thereof) with another member of the complex labeled by any means (e.g., those means described above) are provided to screen for competitors or enhancers of complex formation. In specific embodiments, blocking agents to inhibit non-specific binding of reagents to other protein components, or absorptive losses of reagents to plastics, immobilization matrices, etc., are included in the assay mixture. Blocking agents include, but are not restricted to bovine serum albumin, 13-casein, nonfat dried milk, Denhardt's reagent, Ficoll, polyvinylpyrolidine, nonionic detergents (NP40, Triton X-100, Tween 20, Tween 80, etc.), ionic detergents (e.g., SDS, LOS, etc.), polyethylene glycol, etc. Appropriate blocking agent concentrations allow complex formation.

After binding is performed, unbound, labeled protein is removed in the supernatant, and the immobilized protein retaining any bound, labeled protein is washed extensively. The amount of bound label is then quantified using standard methods in the art to detect the label.

In preferred embodiments, polypeptide derivatives that have superior stabilities but retain the ability to form a complex (e.g., one or more component proteins modified to be resistant to proteolytic degradation in the binding assay buffers, or to be resistant to oxidative degradation), are used to screen for modulators of complex activity or formation. Such resistant molecules can be generated, e.g., by substitution of amino acids at proteolytic cleavage sites, the use of chemically derivatized amino acids at proteolytic susceptible sites, and the replacement of amino acid residues subject to oxidation, i.e. methionine and cysteine.

iii. Cell-Based Assays

In certain embodiments, assays can be carried out using recombinant cells expressing the protein components of a complex, to screen for molecules that bind to, or interfere with, or promote complex activity or formation. In certain embodiments, at least one of the protein components expressed in the recombinant cell as fusion protein, wherein the protein component is fused to a peptide tag to facilitate purification and subsequent quantification and/or immunological visualization and quantification.

A particular aspect encompassed by the present invention relates to identifying molecules that inhibit or promote formation or degradation of a complex encompassed by the present invention, e.g., using the method described for isolating the complex and identifying members of the complex using the TAP assay described in Section 4, infra, and in WO 00/09716 and Rigaut et al. (1999) Nature Biotechnol. 17:1030-1032, which are each incorporated by reference in their entirety.

In another embodiment encompassed by the present invention, a modulator is identified by administering a test agent to a transgenic non-human animal expressing the recombinant component proteins of a complex encompassed by the present invention. In certain embodiments, the complex components are distinguishable from the homologous endogenous protein components. In certain embodiments, the recombinant component proteins are fusion proteins, wherein the protein component is fused to a peptide tag. In certain embodiments, the amino acid sequence of the recombinant protein component is different from the amino acid sequence of the endogenous protein component such that antibodies specific to the recombinant protein component can be used to determine the level of the protein component or the complex formed with the component. In certain embodiments, the recombinant protein component is expressed from promoters that are not the native promoters of the respective proteins. In a specific embodiment, the recombinant protein component is expressed in tissues where it is normally not expressed. In a specific embodiment, the compound is also recombinantly expressed in the transgenic non-human animal.

In certain embodiments, a mutant form of a protein component of a complex encompassed by the present invention is expressed in a cell, wherein the mutant form of the protein component has a binding affinity that is lower than the binding affinity of the naturally occurring protein to the other protein component of a complex encompassed by the present invention. In a specific embodiment, a dominant negative mutant form of a protein component is expressed in a cell. A dominant negative form can be the domain of the protein component that binds to the other protein component, i.e., the binding domain. Without being bound by theory, the binding domain will compete with the naturally occurring protein component for binding to the other protein component of the complex thereby preventing the formation of complex that contains full length protein components. Instead, with increasing level of the dominant negative form in the cell, an increasing amount of complex lacks those domains that are normally provided to the complex by the protein component which is expressed as dominant negative.

The binding domain of a protein component can be identified by any standard technique known to the skilled artisan. In a non-limiting example, alanine-scanning mutagenesis (Cunningham and Wells (1989) Science 244: 1081-1085) is conducted to identify the region(s) of the protein that is/are required for dimerization with another protein component. In other embodiments, different deletion mutants of the protein component are generated Such that the combined deleted regions would span the entire protein. In a specific embodiment, the different deletions overlap with each other. Once mutant forms of a protein component are generated, they are tested for their ability to form a dimer with another protein component. If a particular mutant fails to form a dimer with another protein component or binds the other protein component with reduced affinity compared to the naturally occurring form, the mutation of this mutant form is identified as being in a region of the protein that is involved in the dimer formation. To exclude that the mutation simply interfered with proper folding of the protein, any structural analysis known to the skilled artisan can be performed to determine the three-dimensional conformation of the protein. Such techniques include, but are not limited to, circular dichroism (CD), NMR, and X-ray crystallography.

In certain embodiments, a mutated form of a component of a complex encompassed by the present invention can be expressed in a cell under an inducible promoter. Any method known to the skilled artisan can be used to mutate the nucleotide sequence encoding the component. Any inducible promoter known to the skilled artisan can be used. In particular, the mutated form of the component of a complex encompassed by the present invention has reduced activity, e.g., reduced RNA-nucleolytic activity and/or reduced affinity to the other components of the complex.

In certain embodiments, the assays encompassed by the present invention are performed in high-throughput format. For example, high throughput cellular screens measuring the loss of interaction using reverse two hybrid or BRET may be used and offer the advantage of selecting only cell penetrable molecules (A. R. Horswill, S. N. Savinov, S. J. Benkovic (2004), Proc. Natl. Acad. Sci. USA, 101: 15591-15596; A. Hamdi, P. Colas (2012), Trends Pharmacol. Sci., 33: 109-118). The latter approaches require further validation to assess the “on target” effect. In one or more embodiments of the above described assay methods, it may be desirable to immobilize polypeptides or molecules to facilitate separation of complexed from uncomplexed forms of one or both of the proteins or molecules, as well as to accommodate automation of the assay.

b. Use of Complexes to Identify New Binding Partners

In certain embodiments encompassed by the present invention, a complex encompassed by the present invention is used to identify new components of the complex. In certain embodiments, new binding partners of a complex encompassed by the present invention are identified and thereby implicated in chromatin remodeling processing. Any technique known to the skilled artisan can be used to identify such new binding partners. In certain embodiments, a binding partner of a complex encompassed by the present invention binds to a complex encompassed by the present invention but not to an individual protein component of a complex encompassed by the present invention. In a specific embodiment, immunoprecipitation is used to identify binding partners of a complex encompassed by the present invention.

In certain embodiments, the assays encompassed by the present invention are performed in high-throughput format.

The screening methods encompassed by the present invention can also use other cell-free or cell-based assays known in the art, e.g., those disclosed in WO 2004/009622, US 2002/0177692 A1, US 2010/0136710 A1, all of which are incorporated herein by reference.

The present invention further pertains to novel agents identified by the above-described screening assays. Accordingly, it is within the scope of this invention to further use an agent identified as described herein in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

### XIII. Kits

The present invention also encompasses kits for detecting and/or modulating biomarkers described herein. A kit of the present invention may also include instructional materials disclosing or describing the use of the kit or an antibody of the disclosed invention is a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

### XIIII. Tables 1-4 Screen Hits

Tables 1-4 below list the gene symbols also found in ASCII text files submitted herewith (See “Larges Files” paragraph on page one of the PCT application). Sequences and additional information regarding the screen hits can be found in the large ASCII files submitted herewith. The gene symbols listed below represent well-known and widely used gene symbols. A person of ordinary skill in the art would be able to recognize such a symbol and be able to locate its corresponding sequence in any well-known gene database, including, but not limited to Gene Cards or Ensembl.

TABLE 1
CRISPR Postitive Hits (Gene Symbol)

| Gene | Gene | Gene | Gene | Gene | Gene | Gene | Gene |
|---|---|---|---|---|---|---|---|
| Symbol | Symbol | Symbol | Symbol | Symbol | Symbol | Symbol | Symbol |
| USP7 | RPS27P29 | NPIPB8 | TOPBP1 | MGAT5 | RPL22 | CRABP2 | LGALS7 |
| BCORL1 | RPS27P13 | CCDC138 | RFC3 | MAN1A1 | C20orf27 | ECT2 | GOLGA6L17P |
| SS18L2 | RPS27P9 | B4GAT1 | DDX10 | TRIM36 | ENTPD1 | KMT2C | GOLGA6L5P |
| NEDD8 | ATP5MFP5 | AKAP5 | TUSC2 | ZNF273 | ZCCHC2 | CD96 | LOC392196 |
| RBM4 | ATP5MFP2 | ZNF737 | TP53AIP1 | SULT1A3 | SPART | CDK2 | SEPT5- |
| RBM14- | TIA1 | ERH | HS2ST1 | SULT1A4 | ECHS1 | NAIF1 | GAGE2A |
| RBM4 | | | | | | | |
| MEPCE | SEC61B | TOR3A | DDX23 | SMAD6 | EIF2B3 | TIMM17B | SPDYE5 |
| ASXL1 | CDK1 | ARRB1 | EPS8L1 | RRM2 | PRPF8 | BARX1 | CT45A3 |
| DDX6 | MTBP | PNPLA8 | ZNF41 | VGLL1 | ZNF559- | SPDYE2 | GOLGA8Q |
| | | | | | ZNF177 | | |
| INTS1 | NDUFAF5 | MYOC | KLHL24 | MYL9 | PEX3 | SPDYE2B | HSFY1 |
| CHAF1B | CT55 | DBNL | FMO5 | ABCG5 | FITM1 | SPDYE6 | HSFY2 |
| DTYMK | ZRSR2 | FAM71A | SLC35G1 | GRIK3 | H2AB3 | HCFC1 | GOLGA6L4 |
| ISL1 | SERINC3 | SUZ12 | CANX | STK10 | H2AB2 | TNPO3 | CT45A7 |
| KEAP1 | UBE2Q1 | SLC25A29 | TMEM171 | GABRD | VWF | PRR12 | CT45A6 |
| UBE21 | AGBL1 | PPAN | LENG1 | HSD17B12 | COPB1 | GNGT2 | CT45A5 |
| SUPT20H | TP73 | COX7C | SLIT1 | C14orf178 | CTXN3 | BLZF1 | MIA2 |
| CCNE2 | PEBP4 | NKIRAS2 | PYHIN1 | TMC7 | HNRNPDL | FGF18 | ATP5MF- |
| | | | | | | | PTCD1 |
| CARS1 | ZNF99 | RSRC1 | TDRD10 | GSKIP | DNAJC24 | NPIPB4 | CT45A1 |
| NEDD8- | USP17L7 | CDK12 | FLII | RPUSD2 | ZNF680 | TP53INP1 | RBMY1F |
| MDP1 | | | | | | | |
| RD3 | DHX9 | APOBEC3D | PEMT | TBX10 | RRS1 | MTF2 | RBMY1J |
| MCM7 | MYH9 | ATP5MC1 | NPB | ID4 | KNCN | POTEB2 | TBC1D3I |
| IPO5 | AKTIP | PTPRK | CASS4 | NCKIPSD | NPIPB12 | POTEB | RPL17- |
| | | | | | | | C18orf32 |
| H3C2 | HCN2 | HYAL2 | NPNT | IFT81 | NPIPB13 | CDK13 | GOLGA8O |
| HMGB1 | CLEC6A | THAP9 | KAT5 | APOBEC4 | TRIL | GID8 | TBC1D3L |
| LDB1 | COX7B | COX10 | MAP1LC3A | RTN4 | VCX3A | APBB1 | TBC1D3D |
| UBC | ATP5F1C | GDNF-AS1 | TMEM236 | BIRC5 | GCLC | GNPDA2 | NBPF11 |
| USP17L10 | BIRC6 | RPS20 | PCDHGA3 | SLC7A8 | OPN4 | JOSD1 | TMED7- |
| | | | | | | | TICAM2 |
| PCID2 | CHD7 | PSG1 | MRPS14 | MAGEB1 | TMEM251 | SLC4A9 | MBD3L3 |
| GIGYF2 | CDC5L | TOR1AIP1 | JMJD7- | SH3BP5L | UBAP2 | HOXA6 | TBC1D3G |
| | | | PLA2G4B | | | | |
| FLCN | RNF8 | MVK | TTC14 | ACOX1 | MRGBP | GABRA4 | TBC1D3H |
| LAMTOR1 | ESRP2 | CBX8 | ANP32A | B3GALT2 | ATXN7L2 | GABRR2 | ERCC6 |
| ATP5F1A | LOC441155 | GOLGA6A | ZNF319 | IGFN1 | ITGA9 | WRNIP1 | TBC1D3E |
| TAF5L | ZC3H11B | GOLGA6B | ATP5ME | MPHOSPH6 | STAC | HOXA4 | TBC1D3F |
| MED12 | TMEM259 | ZNF208 | SCNNIB | HTR4 | ANAPC2 | TSG101 | TBC1D3 |
| PRMT1 | KMT5B | INTS2 | STOML2 | MAML2 | MFSD1 | C1orf122 | TBC1D3C |
| POLD2 | ACADM | TMEM69 | IL4I1 | HDDC2 | RBX1 | JAKMIP3 | GOLGA8N |
| SEC63 | SMARCA5 | CDC42EP2 | LARS1 | CLEC2L | OR2T33 | IFT46 | GAGE12J |
| RACK1 | HTT | TFDP1 | HRH4 | PRPF19 | SOX4 | WBP2NL | AMY1A |
| SAE1 | POLA2 | AGAP1 | BOD1L1 | MRPL12 | TAGAP | GJB1 | AMY1C |
| CKS1B | C3orf67 | ADI1 | HMBS | ATP5F1E | ZNF584 | SPATA5L1 | AMY1B |
| PHC1 | LOC730110 | SP100 | FCGR1B | MAP3K2 | SLC38A11 | CIB1 | ANKRD20A3 |
| EDF1 | TLCD3B | NLE1 | TIMP1 | VPS4B | SMC3 | GCG | ANKRD20A1 |
| TADA2B | CDAN1 | NPIPA5 | HMGA1 | CAMK1G | CNGA4 | OR4F6 | ANKRD20A2 |
| RPL28 | SLC39A9 | RAB28 | PPIAL4A | IPMK | CLIP4 | SEC14L1 | ANKRD20A4- |
| | | | | | | | ANKRD20A20P |
| USP17L14P | VPS37A | OR10G7 | WAPL | PPP2R2A | PGK2 | SOCS4 | ANKRD20A4 |
| SLC33A1 | GCNT2 | LRRC43 | MED6 | MASP1 | ZNF236 | CNOT9 | GAGE12H |
| DOCK3 | SFR1 | DHX36 | LRRC37A2 | CNTLN | ZNF83 | OR2G2 | GAGE12C |
| PPP1R7 | LMLN | QTRT2 | ABCD1 | LYAR | PRRT2 | CASP2 | GAGE12E |
| USP17L22 | COPS2 | RPS27P8 | USP17L3 | INO80B | NEURL4 | HPSE | GAGE12D |
| PCGF1 | GARS1 | RPS27 | KRT12 | GSE1 | IFT57 | MAF1 | GAGE12G |
| BRD8 | FRMD4A | CNOT6 | CKAP5 | TMEM232 | TMEM159 | FOXL1 | GAGE12B |
| YPEL5 | INTS12 | PPP6R3 | GOLGA6C | AHDC1 | CASC4 | KRT18 | GAGE12F |
| CDC73 | HMMR | PMAIP1 | EPHB6 | NPIPB11 | PRKX | ZHX1 | |
| USP17L6P | RNF185 | ATP6V1D | ZFAND6 | MDM4 | ARID3C | CKLF | |
| USP17L13 | SLC35A1 | ORC6 | NOP14 | RBM48 | EFCAB2 | WIZ | |
| RBM8A | ARNTL2 | EXO1 | STK32B | EP300 | INPP5F | CAMSAP1 | |
| COPS5 | PA2G4 | CERS3 | C8orf89 | RFC1 | GSTZ1 | TMEM63A | |
| BAHD1 | POLE2 | DPEP2NB | OTUD7B | LEFTY1 | KYNU | CAMKV | |
| SUPT16H | PIKFYVE | MORF4L1 | PRSS48 | COL21A1 | KIAA0930 | SYNPO | |
| TRIM55 | ATP6V1B2 | ASB9 | TPR | ATP1B3 | INO80E | PSMA1 | |
| HMGB1P5 | CNOT3 | SLX1B- | LRRC56 | PGDN | LAMTOR4 | OR5K3 | |
| | | SULT1A4 | | | | | |
| NPIPA7 | MAFA | SLX1A- | CABP7 | SLC8B1 | BICC1 | GNAL | |
| | | SULT1A3 | | | | | |
| NPIPA8 | TSHR | RPA3 | RIOK2 | LRP2BP | MPC1 | ZNF625- | |
| | | | | | | ZNF20 | |
| PAFAH1B1 | ZNF510 | PAIP1 | OSBPL8 | APOA5 | FASTKD2 | ZNF20 | |
| H6PD | PPP1R3C | OR6F1 | RPL15P3 | KCNJ14 | TMEM147 | PPIAL4G | |
| HEXIM1 | SSRP1 | MEN1 | TNFRSF1A | C15orf48 | KIF23 | PPIAL4C | |
| TMED2 | TNFRSF4 | RPA1 | CD3E | TRIM6 | ZNF93 | LIN28A | |
| CYP3A7- | PSMD14 | RPL36A | BMPER | FBXO9 | ZNF37A | KLHDC4 | |
| CYP3A51P | | | | | | | |
| CYP3A7 | DEFB106A | RPL36A- | PKD1P6- | COG3 | CLPX | MAP4K4 | |
| | | HNRNPH2 | NPIPP1 | | | | |
| SEM1 | DEFB106B | LRRC1 | KCNN3 | ACAP1 | CHMP1B | GRTP1 | |
| USP17L30 | DNAJB12 | SLC2A12 | MAP1B | SATB2 | SMG1 | ADGB | |
| USP17L29 | C9orf57 | CLGN | ZNF557 | CSGALNACT1 | RFPL3 | TUBB2BP1 | |
| USP17L26 | OGFR | AURKC | CCL8 | ZNF638 | UMPS | STC1 | |
| USP17L24 | EIF4ENIF1 | STMN1 | SLC25A5 | UNC50 | NOL6 | C17orf98 | |
| USP17L25 | ATP5PF | OXA1L | SULT1C2 | H2AC13 | ZNF341 | WDR36 | |
| USP17L5 | DSC2 | TINF2 | PDCD10 | H2AC14 | EID3 | ACAD10 | |
| USP17L28 | PRIM2 | UHRF2 | GLI3 | SH3YL1 | PPIAL4H | CD58 | |
| USP17L27 | SNAP29 | WTAP | FAM50A | BTLA | PHF10 | COLEC10 | |
| THAP11 | NPIPB1P | RBBP8 | USP17L2 | HLA-DRA | HTATIP2 | PRKAG1 | |
| ATP5MF | HMGB1P6 | TTBK2 | ADH7 | NCAPG2 | FAM160A1 | WDR45B | |
| NTM | HMGB1P1 | TSC2 | DCP1A | ACKR3 | ARL10 | SEMA3A | |
| USP17L15 | HMGB1P10 | PIK3R3 | PTDSS2 | PYM1 | PPM1D | GALNT8 | |
| PTEN | TSR2 | LOC110117498- | NUDT7 | OR4D11 | MAP7D2 | FAIM2 | |
| | | PIK3R3 | | | | | |
| PTENP1 | WDR25 | SNRNP25 | RPF2 | ANKHD1- | CCDC142 | CD46 | |
| | | | | EIF4EBP3 | | | |
| LOC649352 | LIX1 | OR10G4 | HSPA9 | ANKHD1 | DNAH11 | PDCD11 | |
| ZBTB10 | NOP56 | LAPTM4A | DTL | MED19 | CD300LG | NDUFA2 | |
| NFS1 | PUF60 | SKIL | NBPF9 | UTF1 | TMBIM1 | GPR85 | |
| TIMM23 | UFM1 | CUL4B | BTBD8 | ACOX2 | B4GALT6 | CDRT15L2 | |
| RPA2 | RPS27AP16 | UGT2B28 | MBD5 | LAMTOR2 | FUBP3 | CPN1 | |
| KIF18A | MICALL1 | PSMD12 | SFSWAP | SLCO1B3 | MUC13 | TMEM126A | |
| MRFAP1 | ZBTB38 | ZC3H13 | NBPF12 | SLCO1B3- | MPI | NACA2 | |
| | | | | SLCO1B7 | | | |
| DMAP1 | PGA3 | RAP2A | SCNN1A | H2AB1 | OR5L1 | UPK3A | |
| MSMO1 | PCNT | ZDHHC17 | ITGB6 | EP400 | KCNN2 | RPS10- | |
| | | | | | | NUDT3 | |
| TADA1 | PIP5K1B | MAP1LC3C | NLRP6 | SRSF3 | ZNF629 | ORC1 | |
| NPIPB7 | RAD54L2 | PPIP5K2 | FMNL2 | CYP4Z1 | GIGYF1 | ATP5PD | |
| COPE | UBE2L3 | PQBP1 | BTBD9 | FAM49B | SEMA3C | CCNI2 | |
| COMMD1 | LYZ | TANGO6 | CD300LD | TMEM198 | GRM5 | DCP1B | |
| OR10J1 | TTC3 | L3MBTL2 | TMEM68 | TAF5 | TFAP2A | RCSD1 | |
| NPIPB2 | ATP6V1E2 | NDUFA1 | SRPK2 | BFSP1 | C1or21 | NCLN | |
| USP17L18 | GRID1 | PEAK3 | VAV3 | TRPC5OS | TBCK | ERCC5 | |
| USP17L11 | TERF1 | C4orf47 | ITSN2 | LYN | OR51A4 | HMGB2 | |
| USP17L20 | ATP5F1B | POTEC | INTS10 | PRKG2 | ADAM17 | ARGLU1 | |
| RBPJL | TATDN1 | POTEB3 | GPR89B | HIVEP3 | GBGT1 | CDCA8 | |
| SOX11 | THUMPD3 | MED30 | GPR89A | RBM10 | UFD1 | PLOD2 | |
| CANT1 | PODXL | GPALPP1 | SEMA4G | TSR3 | VPS13C | DEFB108B | |
| USP17L17 | LUC7L3 | ING5 | PUS10 | MIB2 | PHOX2B | SCD | |
| USP17L9P | MRPL42 | RPS10 | CFAP57 | ERBIN | KIF11 | BASP1 | |
| CUL1 | USO1 | ACTRT3 | HECTD1 | TMEM101 | PSME3IP1 | OR10G8 | |
| CCN5 | PRPF40A | AR | ENOSF1 | SOX5 | ACOT13 | KDM5C | |
| TAF6L | GLIPR1 | FBXL5 | HNRNPCL1 | NBPF26 | SEH1L | EFCAB9 | |
| TFAP2B | SNRNP40 | ASGR2 | OR10G2 | NBPF1 | OR8G5 | NAA16 | |
| GINS1 | HMGA1P8 | SFRP5 | BTG3 | LOC102724250 | ITCH | COMMD3- | |
| | | | | | | BMI1 | |
| SLCO5A1 | MPC2 | HSD17B4 | MIS18BP1 | GGT7 | MCTP1 | OR10G9 | |
| ZBTB2 | SMARCA2 | ZNF143 | SHISA7 | DUT | ADA | ZNF138 | |
| COPS3 | COL4A5 | ABCA5 | BTAF1 | SRGAP2 | NAA25 | GAGE10 | |
| PKD1P4- | IDO2 | AQP9 | ORMDL3 | HELT | RAB7A | OR51A2 | |
| NPIPA8 | | | | | | | |
| VPS16 | ZNF224 | OR14J1 | TNFAIP1 | PLA2G4B | SUGP1 | TUBAP2 | |
| PHC1P1 | LCAT | NCF1 | PAQR6 | TCTEX1D2 | MED15 | SPANXD | |
| KNL1 | COPS6 | PLEKHH3 | F11 | CD22 | MORC4 | POTEJ | |
| UBE2H | QRICH1 | SERPINB13 | ROMO1 | SRCAP | DDX49 | OCM2 | |
| PLEKHG6 | SLC25A28 | CD44 | CCNI | MXD1 | DCP2 | INTS7 | |
| SFMBT1 | TOB2 | XYLT1 | PPP4C | PTGES | C19orf33 | OR1L6 | |
| ELOB | LSS | NAMPT | PROM1 | HNF1B | ESRP1 | OR1L4 | |
| SPAG1 | GPRIN3 | KL | POLR1E | CATIP | PTPN14 | LOC102723502 | |
| AP2S1 | COX7A2 | H3C15 | RPS3 | SERP1 | RCOR2 | GJA9- | |
| | | | | | | MYCBP | |
| TERF2 | ZC3H12A | H3C14 | PLEKHA8 | RHNO1 | DDX53 | TBC1D3B | |
| ASXL2 | OR10J5 | TOP2A | PKDCC | AMPD1 | STK3 | PCYT2 | |
| HMGCS1 | OR2M5 | ATP8B2 | RWDD4 | ARRB2 | IL18RAP | GAGE13 | |
| SUPT4H1 | PRPF4 | TLCD5 | SLC35E1 | PPIL1 | ZNF726 | TMED7 | |
| DNMT1 | TRIM43 | PDHX | ADGRL1 | LZTS1 | RPP30 | NBPF14 | |
| VIRMA | TBCA | SYAP1 | ZNF563 | NSRP1 | ZMIZ1 | NPIPB3 | |
| HIPK3 | SQLE | PDZK1 | BIVM- | STXBP3 | TACR1 | NPIPB5 | |
| | | | ERCC5 | | | | |
| ING1 | ENY2 | SOD1 | DAP3 | DCDC2B | WNT10B | OR2T29 | |
| ST3GAL3 | C17orf100 | TAOK2 | P2RY2 | AGK | AASDHPPT | JMJD7 | |
| PSG8 | HDLBP | PORCN | CYB561D1 | HTR3E | GIMAP8 | POTED | |
| BCR | UBA1 | KRTAP10-6 | TBCB | LILRB1 | POTEI | ZNF492 | |
| SKP2 | OR7E24 | SCARF1 | LOC102724957 | UTP4 | IFNA8 | LOC102724334 | |
| MOV10L1 | NPIPA2 | SSTR5 | ZNF644 | MGAT4C | SERPINI2 | H2BS1 | |
| LOC100421094 | NPIPA3 | PLAC8L1 | TMEM167A | ATP1A1 | CLDN22 | PMF1 | |
| USP17L19 | ATP6V1C1 | OCEL1 | TMED4 | IQCB1 | ZNF865 | TUBB2A | |
| ASPSCR1 | SMCO3 | XBP1 | OR9Q1 | KLHL34 | SERPINB4 | MBD3L2B | |
| HIC2 | ATP6V1E1 | FTH1 | CBFA2T2 | CMPKI | DNAI2 | MBD3L2 | |
| THAP10 | AARD | MUC12 | DNAJC9 | PTGES3L- | DNAJC19 | ISY1 | |
| | | | | AARSD1 | | | |
| ZNF572 | PLA2G4F | C20orf85 | PSG3 | LRRC34 | C6orf52 | ISY1-RAB43 | |
| SFPQ | GABARAP | METTL6 | FASLG | PLBD2 | PSMD4 | LYZL2 | |
| MGA | TOM1L1 | ZNF98 | TNRC6A | TRAPPC11 | LANCL3 | TRIM6- | |
| | | | | | | TRIM34 | |
| RNF115 | PINLYP | FOXK2 | AVP | RBM18 | FOXP1 | SPDYE18 | |
| TRIP12 | C6orf141 | MYCBPAP | WDHD1 | DDX19B | TIPRL | TUBA1B | |
| SLC35A2 | SIGLEC5 | USP17L4 | TDRD6 | LSR | SCN4B | IFNA7 | |
| AFAP1 | FBXW8 | TNNI2 | CCDC71L | CDC7 | CROT | STX16- | |
| | | | | | | NPEPL1 | |
| UBA2 | RPS27P3 | PLA2G5 | LINC01620 | RAB25 | DSCAM | LOC100288966 | |
| NPIPB15 | RPS27P19 | CUL2 | UQCRFS1 | MED14 | CTCF | SPANXC | |
| TSC1 | VCX3B | MTRNR2L10 | NEFL | SNAPC4 | DPYSL2 | INO80B- | |
| | | | | | | WBP1 | |
| CTSB | NPIPP1 | BMP4 | MRPL13 | SART1 | RNF148 | TBC1D3K | |
| L3MBTL3 | CDR2 | PHB2 | SLC35B3 | CACNB1 | CTSD | OR2T5 | |
| WRB- | MAP3K20 | CHADL | LDLRAD3 | MYH7 | TRA2A | CHMP3 | |
| SH3BGR | | | | | | | |
| C6orf47 | MLEC | UBE2E3 | VPS41 | CD3D | MCTP2 | KRTAP10-4 | |
| CACNA1G | TTC32 | SNX11 | GABRA3 | ATP9A | LMTK2 | GAGE1 | |
| RORA | POLD3 | VPS33A | PLA2G7 | SRSF10 | FERMT1 | ZHX1- | |
| | | | | | | C8orf76 | |
| ASB11 | IFNA4 | TACR3 | CCDC88B | H2BC15 | FAM209B | VCY | |
| SLC15A1 | USP17L21 | PDCD7 | COPG1 | FAM169B | ZNF235 | VCY1B | |
| EED | USP17L12 | NPIPB10P | H2AX | RCHY1 | NDUFB7 | SPRR2D | |
| RFX5 | USP17L1 | ODR4 | ARMCX5 | RPL22L1 | FAM83A | PPIAL4D | |
| RAB33B | SGF29 | NDUFA11 | LARP7 | PPAN- | EWSR1 | PPIAL4E | |
| | | | | P2RY11 | | | |
| CENPW | LIN54 | RPTN | FAM174C | WDR26 | LRRC3B | PPIAL4F | |
| GOLGA6D | HMGN2 | GET1 | ACOT8 | ACTR5 | HIVEP1 | TSNAX- | |
| | | | | | | DISC1 | |
| PIGQ | ATP5F1EP2 | AMZ1 | TSHB | WDTC1 | IGSF11 | SPATA31A3 | |
| CT47A2 | PPA1 | MED28 | USP17L8 | C8orf33 | PELP1 | SPDYE21P | |
| CT47A1 | MFAP3 | MAPK8IP1 | GPS1 | GAST | WDR11 | H4C14 | |
| CT47A3 | ZNF100 | COX6B1 | MGST1 | GRB14 | MCM3AP | H4C15 | |
| CT47A8 | TUBGCP4 | SUFU | SLC12A2 | CCDC39 | BBX | FAM174B | |
| CT47A7 | SLC6A8 | TMEM39A | KBTBD8 | VPS18 | CEP295 | H2BC12 | |
| CT47A4 | ZNF501 | FOXD4L4 | FSDIL | RASGRP3 | IKZF2 | CTAG1B | |
| CT47A6 | STS | UHRF1 | PKD1P5- | LTF | SPACA9 | CTAG1A | |
| | | | LOC105376752 | | | | |
| CT47A5 | H3C11 | NDUFS2 | SNCA | LRRC27 | TMEM127 | KRT6B | |
| CT47A12 | SFT2D2 | UBA3 | MTRNR2L3 | RNF13 | UGT3A2 | LRCH4 | |
| CT47A9 | LRRC52 | DRAP1 | ALAS1 | MOCOS | PCDH7 | ANKRD20A8P | |
| CT47A10 | PSMB1 | PRR20C | MGAT2 | NDUFA6 | SGPL1 | TIMM23B- | |
| | | | | | | AGAP6 | |
| CT47A11 | ELOVL7 | PRR20E | SPDYE1 | SCUBE3 | NDOR1 | PMF1- | |
| | | | | | | BGLAP | |
| RTF1 | RPSAP58 | PRR20B | GABRP | RPL15 | STATH | SPANXA2 | |
| L3MBTL4 | NFX1 | PRR20A | PSMC1 | FOXD4L5 | SMO | SPANXA1 | |
| ATXN7L3 | NPIPA9 | PRR20D | OSBPL11 | ZSWIM1 | PDXDC2P- | NBPF8 | |
| | | | | | NPIPB14P | | |
| IER5 | PKD1P3- | BCLAF3 | CIAPIN1 | TNFSF9 | LINGO1 | C8orf76 | |
| | NPIPA1 | | | | | | |
| POU2F2 | ARAP1 | GTSE1 | PKNOX1 | ANTKMT | DHX30 | CKLF- | |
| | | | | | | CMTM1 | |
| BANF2 | ELP1 | F8A1 | OR4K15 | SIGLEC14 | NEUROG1 | GOLGA6L3 | |
| ARMCX2 | CLEC4C | F8A3 | CAPS | PCGF5 | RAB5B | GOLGA6L10 | |
| INTS5 | IMMP1L | F8A2 | IRF2BP1 | FGF1 | TMPRSS2 | SLX1A | |
| CALR3 | TRMT12 | LIN52 | RFC4 | SPINK5 | H2BC4 | SLX1B | |
| ZNF592 | DEFB104B | RCCD1 | MOB2 | TMEM106C | OXER1 | FOXD4L1 | |
| PARP14 | DEFB104A | CTPS2 | IL4R | TMEM222 | C1GALT1C1 | PABPC1L2A | |
| FOLR1 | DCUN1D4 | ATP5F1D | H2BC7 | GUCY2C | HECA | PABPC1L2B | |
| NPIPA1 | COX7BP1 | PKD1P1 | DDX27 | PGR | USP19 | ARMCX5- | |
| | | | | | | GPRASP2 | |
| RAD51C | BMS1 | RPS27A | TECTA | VPS28 | SHANK2 | TMEFF1 | |
| DDAH1 | KRT33B | PSMB2 | PWP2 | TMPRSS9 | LGALS14 | NBPF10 | |
| LIG1 | TXNRD1 | NOC4L | ARMCX1 | ACTR1B | ZNF607 | COMMD3 | |
| BCL11B | GLG1 | LRP3 | GPR62 | FUT11 | CEP41 | ZNF177 | |
| LOC101927979 | NPIPB6 | EDA | ITIH2 | SEC24B | ZNF773 | TBC1D7- | |
| | | | | | | LOC100130357 | |
| INCA1 | NPIPB9 | DDB1 | GON4L | INTS8 | NCOA5 | LRRC37A | |

TABLE 2
CRISPR Negative Hits(Gene Symbol)

| Gene Symbol | Gene Symbol | Gene Symbol | Gene Symbol | Gene Symbol | Gene Symbol |
|---|---|---|---|---|---|
| TAPBP | RANBP2 | TRAPPC9 | PTCD1 | AP1S2 | SPANXB1 |
| MBTD1 | MUC5B | CYLC2 | ERGIC2 | MON1A | MAGED4 |
| SYS1 | TRAK1 | ZNF404 | TEX46 | ATP6V1G1 | MAGED4B |
| EEF2 | PACC1 | DAZ4 | OR4F15 | ANKDD1B | LOC392196 |
| EIF4E | TOP1 | DAZ1 | RCL1 | GGCX | LY75 |
| SRP19 | CRTC1 | DAZ2 | OR8J3 | ZNF132 | STON1 |
| SYS1-DBNDD2 | BIN2 | DAZ3 | RPA4 | SNX10 | GALNT4 |
| DDX39A | CSMD2 | PSMD7 | SEC13 | ATP9B | POC1B-GALNT4 |
| EIF3E | CYP4F22 | VPS25 | TCOF1 | SMARCA4 | C8orf76 |
| YY1 | TBC1D3H | UFSP2 | RALA | METTL15 | TRIM59-IFT80 |
| SRP72 | TBC1D3G | DDX21 | EHBP1L1 | EPS8 | TNFAIP8L2- |
| | | | | | SCNM1 |
| SRPRA | MARCHF11 | SDCCAG8 | RGS4 | TMEM215 | SCNM1 |
| KANSL1 | NUFIP2 | GTF2B | EXOSC6 | SPECCIL- | ISY1-RAB43 |
| | | | | ADORA2A | |
| ZNF407 | HIGDIC | PPP1R3G | ETF1 | MEPE | ISY1 |
| MROH7-TTC4 | DYRK1A | NTSR2 | OPHN1 | CCIN | PGA3 |
| PPP6C | FGFR3 | ELF4 | DNAAF5 | GPR37 | PGA5 |
| CEBPE | IFI44 | CHAMP1 | RPL31 | GNAZ | SPANXA1 |
| TXNDC12 | CDHR3 | RRAGD | SOST | SCRG1 | SPANXA2 |
| RAB10 | BRF1 | PIP4K2C | ZNF479 | TESMIN | CGB7 |
| SRP68 | MYH4 | INVS | SDR16C5 | EMX2 | NT5C1B-RDH14 |
| IRF1 | ZNF705E | SSBP4 | SLC30A9 | SIAE | USP17L2 |
| SERINC5 | CHURC1-FNTB | E2F3 | DMAC1 | ARHGDIG | PGA4 |
| PDILT | GPR37L1 | TRMT10A | DHX57 | CYP1B1 | OPN1MW2 |
| BAP1 | NLRP8 | KRTAP10-12 | ZBTB7A | HSPB2 | OPN1MW3 |
| SMIM12 | OXCT1 | AP4M1 | PPIAL4E | CEP55 | OPN1MW |
| KRT75 | KCNE2 | CLEC4F | PPIAL4F | PTK2B | USP17L1 |
| ZC3H11A | LOC105372791 | PRKAR2A | SAPCD1 | BAG4 | USP17L21 |
| INHBE | PRPF38B | TAF2 | GPR137 | SCGN | USP17L12 |
| KCNK1 | SNRPC | FBXO47 | CXorf56 | RAB37 | ARMCX5- |
| | | | | | GPRASP2 |
| ICK | BAAT | H4C2 | PHF3 | GUK1 | USP17L4 |
| KTI12 | CD36 | NOS1AP | ELF2 | SNAP23 | LY75-CD302 |
| ALMS1 | KCTD4 | PAXX | TPMT | NMNAT1 | USP17L18 |
| EIF3I | SUCNR1 | TSNARE1 | ATG5 | IRAK1 | USP17L11 |
| FAM76B | MCOLN3 | ECD | TSHZ3 | B9D2 | USP17L17 |
| ARMH3 | PRSS23 | FAM187B | RNF113B | ANGPTL7 | CBWD2 |
| IFI30 | TMEM202 | SMARCB1 | PCDHGB5 | CDC42SE1 | JMJD7-PLA2G4B |
| RPTOR | SLC41A3 | CNOT6 | ZBED5 | PTPRU | USP17L19 |
| SCAP | GOSR1 | ZNF160 | CCDC42 | RALB | USP17L13 |
| PITPNB | VRK2 | NOP58 | TBC1D3K | SIKE1 | VCX3B |
| FAM89A | CLRN1 | SEMA5A | EIF4H | ZNF345 | FOXD4L5 |
| ZUP1 | ABRAXAS1 | FRAT1 | GRK6 | TUBB6 | STON1- |
| | | | | | GTF2A1L |
| EBNA1BP2 | UBE2J2 | ELOVL2 | GPR34 | GPS2 | USP17L8 |
| CCDC144A | TVP23C | ENTPD3 | OR4X2 | FIP1L1 | USP17L3 |
| RGPD6 | C15orf65 | WNK1 | OR51A2 | IQGAP1 | VCX2 |
| RGPD5 | EIF3D | LRRC25 | FAM207A | ADO | VCX3A |
| HLA-DOA | CD46 | H1-1 | POGZ | SHROOM1 | USP17L14P |
| ABALON | ADGRG1 | CFAP97D1 | SAXO2 | SLC17A5 | VCX |
| BCL2L1 | CCDC167 | PDGFRB | TMEM67 | WSB1 | USP17L10 |
| UBE2N | DBP | KIAA1328 | NLRP14 | SDHC | |
| CFAP69 | RBM25 | ZCRB1 | RBMX | GLRX2 | |
| ZNF251 | C9orf24 | KIF1B | COQ5 | SPNS2 | |
| NUP155 | CT45A10 | NCAPD3 | SMIM10 | PIWIL3 | |
| ADSL | KDM4A | ST3GAL1 | CFAP161 | SCAPER | |
| OTUD6A | CCNY | PALM | CYBA | RAB11A | |
| RPL37 | SSX7 | TTN | HOXB5 | NUTF2 | |
| ITGB7 | GPR152 | TOMM20 | IFNW1 | CSNK2B | |
| NUDC | ISG15 | SMIM10L1 | ZCWPW1 | KCNJ5 | |
| MBTPS2 | KIR2DL1 | OTUD4 | DLGAP1 | TMEM233 | |
| BUD31 | MAP1S | CMPK2 | TEKT3 | ADAM9 | |
| ITPRIPL2 | NCKAPIL | ZNF770 | VPS4B | CARHSP1 | |
| SPAG7 | FANCC | MMP3 | CD300LB | RNF146 | |
| CDK9 | TSPAN19 | OR2S2 | TNFSF4 | HMGCLL1 | |
| CT45A7 | NUP160 | WDR59 | GABRB3 | DDX47 | |
| CT45A5 | HNRNPH1 | DDX3X | SP1 | EBAG9 | |
| CT45A6 | TMEM273 | CCR5 | ARPC4 | AQP1 | |
| TTC4 | CCT2 | NRM | PNLDC1 | VMP1 | |
| EIF4G2 | GMPPB | PDPK2P | FNDC4 | TSNAX | |
| FZR1 | OTOP3 | SHTN1 | RYR2 | GGPS1 | |
| NDUFA7 | CALCA | MLF1 | RASA4B | ZNF516 | |
| NUP54 | MKRN3 | ADGRG4 | RASA4 | PSMB9 | |
| CYB5R2 | SEC23A | RWDD1 | RGL3 | DNAJC11 | |
| RPL13 | SYNCRIP | SATB1 | PRRC2C | CATSPERD | |
| PIK3R5 | SOWAHD | GLP1R | CREBZF | ZNF79 | |
| SAP30BP | EGLN3 | SPATA31E1 | CHGB | SMG5 | |
| KBTBD2 | ANKLE2 | TM7SF2 | C1orf195 | OCIAD2 | |
| SAYSD1 | ZNF221 | BMT2 | SYT15 | ZNF195 | |
| NUDT9 | KNSTRN | OR9G4 | SLC4A8 | TSPAN6 | |
| RTKN2 | UBLCP1 | PEX11G | KDM3A | ERC1 | |
| POLR3B | COX19 | LRRC63 | METTL23 | SLC35G2 | |
| LRRC14 | HAUS3 | C8orf37 | PSMD1 | GNAL | |
| KDM1A | NPIPB11 | TECRL | ANKDD1A | OR2K2 | |
| WFDC6 | PDRG1 | PTH2 | GEMIN8 | NCL | |
| TGFBRAP1 | MSL3 | ULK4 | SPINK13 | ABCA4 | |
| CALR | SLAIN1 | FOXH1 | MXRA7 | CCDC27 | |
| ADAT1 | GAL | TSACC | LOC100506055 | GIP | |
| GNRH2 | TEDDM1 | TVP23C- | GRM3 | KCTD1 | |
| | | CDRT4 | | | |
| HARS1 | ADAMTS18 | NLGN3 | CASP4 | FRS3 | |
| ICE2 | CCDC88B | SELENOW | ATP2A3 | ELAC1 | |
| SMPX | COL6A6 | CDC37L1 | ODF3L1 | GPER1 | |
| C5orf24 | EIF3C | VARS | IRF2 | NUP37 | |
| NKX1-2 | EIF3CL | KBTBD12 | SULT6B1 | LBX1 | |
| PFN1 | EZR | PGLYRP2 | LIG3 | CYB561A3 | |
| BCL2 | DOHH | LGR5 | PANK1 | GFPT1 | |
| PCDHB13 | NRG1 | OR8B4 | LRRC47 | NEUROD1 | |
| MTOR | FKBP3 | ZNF813 | TBCB | SMCO2 | |
| CRYL1 | PTHIR | ZC3H12C | FANCG | THBD | |
| TNRC6B | TMEM59L | ADHIC | ADAM12 | LAMA1 | |
| MBTPS1 | RPE65 | RHOT1 | PI4KB | SELENOM | |
| CWF19L1 | CYP4F11 | PTPRH | AMPD3 | PRND | |
| ALG1L | ZDHHC5 | NME1 | EXOSC8 | IQCJ | |
| MOBP | MBNL3 | AP1M1 | KCTD13 | ASTE1 | |
| WSB2 | MXRA5 | ZDHHC9 | SERPINB4 | DSN1 | |
| EIF5A | DST | PRICKLE1 | RSF1 | TVP23B | |
| RNGTT | MTRNR2L6 | CISD1 | APOBEC2 | CRYZL1 | |
| PSMG2 | TRIM34 | RNF145 | OR52N5 | CEP128 | |
| IL7R | ASAP2 | NISCH | PMM1 | ZNF728 | |
| CIC | ASMTL | ENKUR | FABP2 | FBXO25 | |
| OGT | MEIS2 | KIAA1656 | SLC9C2 | XXYLT1 | |
| EXOSC10 | TMEM8B | DYNC1LI2 | IL10RB | MT3 | |
| BACH2 | PPFIA1 | COG4 | CYP4Z1 | NFIX | |
| BDP1 | CLSTN2 | AP2A2 | HLX | TMEM211 | |
| DNMT1 | H4C3 | BTBD7 | ZFYVE26 | LPA | |
| MICALL2 | ZNF814 | NYAP1 | NQO2 | RFXAP | |
| PF4V1 | TRIM48 | EXOSC4 | PTCH2 | SLC34A1 | |
| LMAN2L | ERE | ANKRD28 | SLFN12L | USP10 | |
| SYN2 | ATOH1 | NAXD | SPZ1 | GJA5 | |
| DIDO1 | ZNF567 | KRT15 | PLCD3 | C11orf58 | |
| PYCARD-AS1 | MSL2 | CRLF2 | NEK6 | CTSF | |
| BHLHE22 | NUDCD3 | CARNS1 | INA | CNNM2 | |
| ZSWIM3 | ELOVL1 | OR4K5 | FAM222B | ICMT | |
| RP1L1 | HOOK3 | EHD1 | CLDN19 | U2AF1 | |
| UBXN4 | ARFRP1 | ZNF517 | ADRA2C | U2AF1L5 | |
| CYP2U1 | NUS1 | CLDN24 | CDC26 | LRRC8B | |
| OLFML2B | GRXCR1 | NACA | CBWD6 | FCER2 | |
| TBC1D3P2 | VPS8 | PROM1 | TMEM53 | H2BC3 | |
| LSM2 | TEK | ANKLE1 | BLOC1S3 | RDX | |
| NUP42 | CGB2 | POLR3H | ZNF213 | TDGF1 | |
| TLE2 | CGB3 | C1orf43 | PATL1 | PRPF3 | |
| MSMB | CGB8 | NUP205 | SLC2A13 | DNHD1 | |
| SEZ6L | CGB1 | TIMM44 | ZNF493 | ALB | |
| CDC16 | CGB5 | TMEM31 | M6PR | ACTL6A | |
| RHEB | LINC02210- | POLG2 | ETV3 | IKZF3 | |
| | CRHR1 | | | | |
| EIF4G1 | S100A1 | SMG9 | DAPK2 | SYT13 | |
| PDPK1 | OR51A7 | EAF2 | SLC22A13 | PLEKHA6 | |
| TMEM186 | STK19 | RELA | CSNK1A1 | ANKRD10 | |
| DCAF7 | TPT1 | FRA10AC1 | VPS13D | IL1F10 | |
| NPW | PDE6D | CXorf49 | MAPKAPK5 | TTF2 | |
| HAAO | SLC7A3 | CXorf49B | PIK3AP1 | ADCY4 | |
| TNFSF11 | EIF3M | LRIF1 | C9orf129 | C7orf57 | |
| HLA-DPA1 | KCTD12 | ABRACL | MTMR2 | VSTM5 | |
| B2M | TAF15 | MPRIP | EHBP1 | OR2A12 | |
| APOL6 | CAPN3 | ETHE1 | CD109 | SLC35C1 | |
| MAGEE1 | SLC5A8 | CAPN2 | SH3D21 | TTC38 | |
| HABP2 | ANAPC15 | ARNT | PRMT7 | DOCK2 | |
| DMAC2 | YBX1 | HAS2 | OAS3 | HARBI1 | |
| AACS | SLC9A8 | HSD3B2 | SIOOB | EIF3G | |
| CRIP3 | KRT26 | SLCO2A1 | SMC4 | PTPRD | |
| TCF24 | ASAH2 | EFNA1 | SBK1 | DCAF13 | |
| LRAT | ALS2CL | CSE1L | KLRC1 | DIPK1A | |
| TBC1D3J | ASCL1 | HTR3C | MMEL1 | PCDHB2 | |
| ZNF619 | GPR107 | ZNF806 | P3H2 | COL4A1 | |
| NTHL1 | NSFL1C | TOMM34 | BRD2 | GPR85 | |
| TMEM207 | CBR1 | ANKMY1 | EPB41L1 | CHGA | |
| ACSM1 | NRG2 | MANEA | ACTRT1 | ADAM11 | |
| DBR1 | SLC35B2 | KDSR | EMC8 | TRIM33 | |
| SPANXD | MARCKSL1 | DARS1 | L1CAM | UBALD2 | |
| PPP1CB | KDM3B | RPS21 | AADACL3 | PCEDIA | |
| RAB11FIP2 | LRP5L | TBC1D3 | SCAI | CHORDC1 | |
| SCRN1 | ZNF792 | TBC1D3C | GPN1 | NANP | |
| ZNF736 | STAMBPL1 | TBC1D3E | TTC5 | METTL16 | |
| CWC22 | SEMA3E | GSG1L | GLRB | YEATS2 | |
| CNTROB | EIF2S2 | FLJ44635 | BRF2 | ZNF623 | |
| ABCB4 | NXPH3 | ADGRE1 | COL8A1 | RNF222 | |
| TCF3 | KLRD1 | RCN1 | BRDT | DOCK1 | |
| IAPP | PLD5 | KIAA0100 | PHACTR1 | FBXW5 | |
| ERG28 | MRPS15 | YIPF6 | NKPD1 | FCN3 | |
| ABCC6 | TBC1D3F | XPA | WBP11 | KLHDC7B | |
| SRP54 | ZKSCAN7 | ABCC8 | ADAM32 | NUP85 | |
| EIF3F | RGS18 | TMSB10 | ZPR1 | ARPC2 | |
| EPHX1 | CRISP2 | MPPED2 | USP48 | KCTD20 | |
| AFP | WIPE | FFAR4 | HOXC5 | OR2T34 | |
| EIF3H | H3C7 | RAPGEFL1 | DUSP8 | TLCD4-RWDD3 | |
| GPC5 | GZF1 | BARHL2 | GPRC5A | MOB4 | |
| CLEC4A | BRD4 | MTRNR2L7 | ZNF540 | ATP5MF-PTCD1 | |
| CCDC36 | WDR63 | MTMR6 | MYH7 | CT47A5 | |
| KCNIP2 | ADRB1 | CRHR1 | CXorf58 | CT47A9 | |
| EXOC3 | NAB1 | MMP27 | AMZ2 | CT47A6 | |
| REN | REEP2 | TRIM29 | GOLM1 | CT47A1 | |
| SLC25A51P3 | CLU | PCDHA9 | COL10A1 | CT47A2 | |
| SLC25A51P2 | VAMP4 | PPIAL4A | S100A16 | CT47A12 | |
| SLC25A51P4 | SLC35E4 | C1orf56 | SERTM1 | CT47A3 | |
| COPG2 | VWA8 | LMNA | SGCD | CT47A11 | |
| CCDC157 | MRM2 | CCDC74A | TLE3 | CT47A4 | |
| EXOC5 | MRGPRD | FBRSL1 | PDP2 | CT47A10 | |
| ATXN2L | RPN1 | DGKB | RAB27B | CT47A7 | |
| PSORS1C2 | SGTB | CDKL5 | CYFIP1 | CT47A8 | |
| PYCARD | TBC1D3I | MYLK | S1PR2 | RAB4B-EGLN2 | |
| CT45A3 | EFNA4 | RBAK | SH3BP5 | MIA-RAB4B | |
| PRPF4B | ABAT | RPL38 | MPZL2 | H2AC21 | |
| ABCF1 | TTYH3 | ZBTB14 | GNG11 | RFPL4A | |
| PLSCR2 | UBFD1 | TMEM43 | ALDH18A1 | RFPL4AL1 | |
| ALYREF | NMB | ADAMTSL3 | RAEI | TCEAL6 | |
| STRN4 | FBXL13 | CDKN1B | PLPBP | NME1-NME2 | |
| CT47B1 | H3C12 | ALDH8A1 | ENDOD1 | CMC2 | |
| IDH3G | CPNE2 | DALRD3 | APEX2 | TLCD4 | |
| TMEM164 | PRRC1 | DNTTIP2 | EBF2 | OR10H2 | |
| ACTR3 | ATOX1 | GCC1 | C11orf45 | RPL17-C18orf32 | |
| PHOSPHO2 | ELOVL6 | MIA2 | PTMA | ZNF417 | |
| XPR1 | TFEB | RABIF | FOXA1 | OR2T3 | |
| REPS1 | PRKRA | CARD17 | SNAPC2 | PPIA | |
| SPANXC | SLC25A51 | PRKAG2 | RPS10-NUDT3 | PLGLB2 | |
| ACVR2A | C11orf96 | KRTAP4-11 | NFKB2 | PLGLB1 | |
| BRK1 | SHCBP1 | RIPOR1 | ASB16 | C3orf52 | |
| GCHFR | DISP1 | OR5B17 | ANKRD53 | SSX3 | |
| TMEM41B | SLC52A1 | CCDC122 | ZHX3 | BOLA2-SMG1P6 | |
| TEX35 | FICD | KIAA1671 | MARCKS | BOLA2 | |
| SLC39A7 | HLF | ZNF812P | HIKESHI | CEP68 | |
| CT45A9 | CDK10 | ZNF614 | ZNF117 | ZNF726 | |
| CT45A8 | ASPM | CSNK2A3 | ERV3-1- | ALG11 | |
| | | | ZNF117 | | |
| CT45A2 | CARD6 | FBLN2 | DEPDC1B | ESS2 | |
| MYO5C | MTG2 | SLC1A6 | C6orf15 | NUDT3 | |
| CAP1 | TP53BP1 | IL31 | MKI67 | FNTB | |
| RGPD8 | SYVN1 | MYO1E | ATP2B2 | OR11H1 | |
| CLCC1 | RAB11FIP1 | TMEM125 | TMEM120A | OR11H12 | |
| TBC1D3B | MROH7 | MBP | SEPTIN3 | TRIM6-TRIM34 | |
| CRYGA | INTS3 | MCOLN1 | DHX15 | ITFG2 | |
| PSMB4 | RAB4B | LAG3 | TBX6 | PPIAL4H | |
| TNFRSF10D | SLC6A9 | IL23A | HOXA2 | RABL2B | |
| MMACHC | C1QBP | NKAP | TMEM71 | SPACA5B | |
| UCP3 | MMADHC | GRAMD1A | DLX6 | SPACA5 | |
| SSR3 | HAND1 | WDR33 | CHST7 | SERF2-C15ORF63 | |
| KCNA2 | CYGB | ADAM33 | KRTAP20-2 | CBWD5 | |
| C19orf48 | PPIAL4C | FAM151B | CARD10 | CBWD3 | |
| FXR1 | PPIAL4G | UFM1 | WDR72 | MZT2A | |
| FEM1C | NOD2 | HSFY2 | DOCK11 | GOLGA6L6 | |
| IQCF3 | LIMS2 | HSFY1 | SYNPR | HBA1 | |
| PBK | TMCO4 | S100A9 | TTC37 | HBA2 | |
| GH2 | PIK3CA | HPS4 | PJVK | ZNF664-RFLNA | |
| OR8D2 | QARSI | AKNAD1 | TOMM40 | RFLNA | |
| CT45A1 | ZNF776 | RAB11B | RASSF5 | FAM120A | |
| IFNA1 | NOP53 | RPL7 | SNX27 | OBP2A | |
| IFNA13 | ARPIN-AP3S2 | CPNE9 | GPR17 | VCY1B | |
| COQ10B | OR7D2 | CLSTN3 | TMEM245 | VCY | |
| TKFC | CDK11B | SMPD2 | GALR2 | MSH5-SAPCD1 | |
| ARPIN | CD14 | SRM | CNOT10 | RABL2A | |
| SAP25 | IRF2BPL | CCDC160 | ICAM5 | PLA2G4B | |
| NUP214 | GPR4 | CCDC88C | DHFR | SAA2-SAA4 | |
| POLR1C | FCRL3 | SENP2 | ARF6 | NT5C1B | |
| KLHL1 | IRGM | SLC17A8 | NRXN2 | SEM1 | |
| THEMIS | AIRE | B3GALNT1 | CYB5R4 | TSNAX-DISC1 | |
| GTF3C1 | AQP2 | KAT8 | CLIC5 | GPRASP2 | |
| OR2AG1 | ZNF266 | CDH19 | DCBLD1 | MAP1LC3B | |
| C17orf107 | A4GALT | STXBP5 | USP28 | RPL17 | |
| TBC1D3L | NUP88 | CNR1 | ABHD3 | OBP2B | |
| TBC1D3D | OTOF | ZFP82 | MECOM | ARPC4-TTLL3 | |
| NUB1 | KIAA1614 | RCN3 | TEX13B | TRIM49D2 | |
| SELENOH | ENPP1 | NATD1 | RNF40 | TRIM49D1 | |
| NUP133 | ADAMTS3 | RBPMS | DHX8 | PPIAL4D | |

TABLE 3
ORF Positive Screen Hits

| Gene Symbol | Gene Symbol | Gene Symbol | Gene Symbol | Gene Symbol | Gene Symbol |
|---|---|---|---|---|---|
| IFNG | KCNJ10 | TBPL1 | GPRIN3 | GYG2 | NO_MATCH_146 |
| IFNA21 | TMEM98 | CRYBB1 | NO_MATCH_127 | GNGT2 | FAM74A1 |
| FCGR2A | CBR3 | ARL13B | BET1 | CCNC | HIST1H3B |
| ATP6V0A1 | ZDHHC13 | TIMMDC1 | POLR2B | VPS36 | TFDP3 |
| SPATA25 | MYL3 | MASTL | RTP2 | PCDHAC2 | FBXO42 |
| CDX2 | CDKL2 | LINC00588 | LRR1 | XLOC_l2_004840 | ZBBX |
| HOXB13 | Trim72 | MRPS17 | PQLC1 | MTMR12 | C1orf87 |
| IFNGR1 | SLC39A6 | NUDT21 | EIF4A2 | OR6B1 | RUFY4 |
| FCGR2B | GGN | ZNF155 | ADCK2 | HPS5 | IST1 |
| HLA-B | VPS33A | MRFAP1L1 | RBP4 | RLF | RFFL |
| SOX15 | XIAP | EBNA1BP2 | BPGM | ATP6V0C | ARF1 |
| IRF2 | TIMM21 | ENDOD1 | PRL | LXN | CCL26 |
| LHX4 | PCDHGA6 | JUN | USB1 | WASF2 | ZBTB14 |
| TLR7 | BCKDHA | ING5 | MRGPRX3 | HIST2H4A | CSK |
| POU5F1 | C10orf82 | UBOX5 | SCPEP1 | XLOC_l2_005151 | IQSEC1 |
| OVOL2 | ACP5 | FMOD | DIABLO | NO_MATCH_139 | ATP5G3 |
| FOS | KLHL26 | NAB2 | DKKL1 | IMMP1L | PTPRCAP |
| IFNA8 | EFEMP1 | NMI | RETN | MRPL46 | ABCC11 |
| IFNA6 | TNPO2 | GAD1 | ASS1 | FAM120A | NDUFV2 |
| TIRAP | CREB3 | PPIL4 | EDF1 | SPANXN3 | TOR1A |
| USO1 | TCEAL9 | TSPEAR-AS2 | NUP62 | SIRT1 | HBG2 |
| FOSL1 | LOC101060386 | ZNF501 | FIBIN | RNF183 | STRADB |
| BMS1 | VSIR | SPO11 | TTC29 | EMB | BTNL8 |
| IFNA5 | ATRNL1 | AGO2 | CT83 | AURKC | FAR2 |
| ANO4 | PDIA4 | LCTL | SGPP2 | FLJ33534 | PTPN9 |
| DLX2 | URGCP | PLEKHA8 | ADA | RARB | CAMK4 |
| HOXA6 | SLC12A7 | DTD1 | MC1R | CAPZB | PIP5K1A |
| RUNX1 | DOK3 | CACNB1 | RAB41 | PIN1 | RPL31 |
| IFNB1 | KRTAP5-6 | KIAA0141 | HIST1H2AJ | HIST1H2BD | CD302 |
| EDA2R | SNX16 | LRRC3B | PAPOLB | GCNT3 | C11orf57 |
| SOX2 | CSF1 | RPS6KC1 | YIPF4 | CCL19 | TPK1 |
| IFNA10 | SLC17A8 | NO_MATCH_118 | PPP4R3A | MPZL1 | ECHS1 |
| GATA3 | PNO1 | SLC28A1 | SLC35A1 | RAB9B | RNF25 |
| DMRT1 | PCDHB16 | KHDRBS2 | UPF3A | ABTB1 | NFKBID |
| IFNA2 | USP6NL | SFT2D2 | GJB3 | MRPL35 | ASB2 |
| MKX | PITRM1 | TREML1 | RNF19A | MAP4K5 | RASSF1 |
| NDN | ACOT11 | KCTD7 | FBXL16 | SULF1 | NO_MATCH_97 |
| HLA-C | HCLS1 | ZBTB9 | SPAG7 | C19orf44 | ENSA |
| NR5A2 | FAM124A | ART4 | HMBOX1 | CFAP97 | HIST1H3H |
| DNAJC5 | CCDC112 | HLA-DQB2 | OR5L1 | RPS6KA1 | PCDHGC3 |
| FOXA3 | CNPPD1 | IGFLR1 | GOSR1 | RIOK1 | RHOQ |
| ZC3H10 | OR51E2 | ZKSCAN3 | PIGF | Rnf150 | CNIH2 |
| TFEB | DRAM2 | TMEM14C | TSPYL5 | HDAC6 | NDUFS5 |
| ECD | PSRC1 | EIF4E2 | HLA-DQA1 | PLPP3 | SEC22B |
| ZFP36L1 | MSL1 | ERLIN2 | PRKCD | CSTA | GAN |
| OOSP2 | USH1C | TANGO2 | RNF2 | PHF13 | ABHD17B |
| ZNF317 | NUP43 | ATP6V0A4 | CAVIN4 | FMO1 | GPR151 |
| CXorf67 | IL17RE | UQCC3 | APOBEC4 | SMPDL3A | HLA-DMB |
| TSPY10 | HOOK1 | TSPAN18 | BHLHA15 | TRAPPC12 | PDZD9 |
| DLX1 | JPH4 | ABRAXAS1 | PHKG1 | PLK2 | HAVCR2 |
| KLF4 | KIAA0930 | TSEN54 | OR2F2 | AGBL5 | DHRS1 |
| IFNA17 | CRYGS | PGRMC1 | ACTR3 | RABL6 | PAGE3 |
| MTR | EVL | FBLN7 | 6-Sep | NGB | EGFLAM |
| DLX5 | FAM_172_A | ESAM | CCDC90B | TRIM28 | GLE1 |
| PIM1 | FAM228A | EMC2 | CTRB1 | MBIP | NO_MATCH_100 |
| YY1 | RXFP2 | LPGAT1 | PPAN | EIF4E | ELMO3 |
| HOXC8 | RHBDF1 | ACTR8 | GEMIN8 | MRPS18B | C7orf13 |
| HOXB6 | ALDH1A2 | CKB | AMMECR1 | SOST | MPST |
| LRPPRC | SYNGR1 | SEMA6C | DUOXA1 | GATC | LILRA3 |
| SOX14 | HILPDA | PML | GNG10 | C2orf15 | CELA2A |
| EGR2 | DNM1 | ACBD4 | NECAB3 | NPC2 | GLMP |
| MAGEA10 | LUC7L | ABCE1 | HBEGF | LIPE | BGN |
| DLX4 | AQP2 | TBK1 | LOC642249 | BDH2 | AXIN2 |
| MCM6 | PPP1R13B | GTSF1 | FUT10 | TNFAIP2 | DCK |
| CEBPE | VIM | TRAF1 | FNIP1 | ILF2 | PIP4K2C |
| DLX6 | PIK3CB | ADGRF2 | OR5H6 | BTBD9 | GNPTG |
| MAGEA9 | NUDT3 | ESRRA | VMO1 | HLA-DRB1 | FAM3A |
| IFNW1 | FZD2 | CSH2 | LEFTY1 | UBXN4 | MEIOB |
| H2AFB3 | SYT16 | DEFB4A | NXPH3 | CDSN | PPP6C |
| DLX3 | HLA-DRB3 | NTNG1 | NYX | C1QBP | LETMD1 |
| UGCG | LMLN | ATF3 | SCARB2 | GOLGA8G | SCGB1A1 |
| BSND | ZNF705D | ZBTB12 | GINM1 | KIAA0040 | RRM2 |
| IFNL2 | SLC39A12 | NARS2 | POMK | MSRB2 | NCOA7 |
| KATNAL1 | ADH1C | NSG1 | SSMEM1 | KLRC2 | ACTR3B |
| RUNX3 | STXIB | PITHD1 | TIMM29 | HIST1H4F | LGALS4 |
| IPO5 | MON1B | IL36RN | SC5D | SNRPA1 | MGAT4C |
| FCGR1A | SUCLG1 | PPP2R3B | SAT2 | OR6T1 | CLDND1 |
| NKX2-5 | EPHB3 | TRMT44 | C10orf107 | SNX27 | STAMBP |
| GATA2 | LRRC37B | KCNIP3 | TMEM67 | PSPC1 | SERHL2 |
| SLFN12 | ZMYM3 | POMP | SYTL5 | MYEOV | MMGT1 |
| FGFR3 | KSR2 | H3F3B | ADCY4 | NO_MATCH_202 | TCOF1 |
| KAT2A | DENND5A | GOLM1 | SLC25A35 | FAM213A | RNF152 |
| PRDM14 | HERC6 | PEX7 | A4GNT | DNAI2 | MPP1 |
| H2BFWT | GDPD5 | STN1 | GATD1 | GTF2H5 | Nrbp2 |
| FOSL2 | FCHO2 | PEX16 | CDCA7L | FGL2 | PSME1 |
| NTRK2 | GPRC6A | G6PC3 | TECR | GLB1L3 | WNT1 |
| SRSF8 | ZNF532 | TSNARE1 | PRPF4B | TMED9 | PDXK |
| HLA-A | YES1 | COG2 | AIDA | CLDN18 | MSH4 |
| VPS26A | DPP3 | LSM2 | HAX1 | RBM11 | CACNB4 |
| CD40 | GAA | TNFSF14 | MFSD13A | GPR155 | CAPN8 |
| MAP3K14 | DUS2 | JAK3 | LINC00346 | PLIN3 | ARPC1B |
| NR5A1 | CNPY2 | 1-Mar | ARL4C | GDF10 | RCE1 |
| ROS1 | SFPQ | SIGLEC7 | LINC00173 | G3BP1 | TDRD9 |
| HPN | CSTF2T | NCAPH2 | FIBCD1 | OPTC | SEMA4B |
| EMILIN2 | FBXL19 | PRR7 | NOXRED1 | SCYL2 | CRIP2 |
| PHF23 | ZDHHC17 | SNRNP48 | CASP5 | IL17RC | ANXA9 |
| RBM47 | GRAMD1C | RAB40C | CCDC172 | PARP6 | CRBN |
| ZFYVE1 | ELP2 | SAAL1 | C4orf33 | NDUFB6 | TADA3 |
| PITX1 | IL1RL2 | AGPAT3 | TBCK | LYPD6 | PHF21B |
| WDR91 | PPM1E | TH | BCL2L10 | GRM8 | EPB41L4A-AS2 |
| FAM43A | GPBP1 | RHOBTB2 | THSD4 | TXNRD3NB | HDGFL2 |
| CPEB2 | TEKT2 | C8orf37 | TUBG1 | EP400 | OR2H1 |
| TBC1D10B | CCDC33 | PIPOX | AKR1C4 | TPP2 | TXNDC9 |
| POLD1 | SETDB2 | ESR1 | RBM15B | OBP2A | NOP53 |
| PITX2 | FOXN3 | MUSK | TCTEX1D1 | TP53 | ZNF277 |
| PRR15 | CLIC4 | CT45A3 | SEC61A1 | NBPF19 | ZNF839 |
| MARCKSL1 | SMAD5 | DNM1L | RPP25 | HTR3E | PLD3 |
| AAK1 | DNAJA4 | TPPP2 | U2AF1L4 | CSNK2A2 | FUCA1 |
| NR2F6 | EHD4 | ETNK2 | TCTN2 | GPC3 | TIMD4 |
| RBAK | LIPH | PPY | SP4 | GNA14 | EEF1AKMT2 |
| TBX22 | ZFP2 | GFAP | ABHD12B | FAHD2B | KLB |
| HNF4A | INTS6 | QPCTL | SLC25A2 | HIST2H2AA3 | NAGK |
| SATB2 | MRPL19 | NO_MATCH_84 | CUEDC2 | DPH3 | ALDOC |
| PPP4C | ADORA2A | CALML3 | GPX2 | FBP1 | DDX19B |
| TMCC1 | TNFRSF10D | CST4 | TGFB2 | PNPLA1 | XPNPEP1 |
| CDX4 | NUDT4 | ANXA4 | TNNI2 | BRI3 | SPATA2 |
| OR2S2 | NPIPB9 | ERO1B | BTRC | HSD3B2 | LOC641367 |
| SPATA31E1 | RFXANK | COL21A1 | NO_MATCH_152 | KRTCAP3 | MATN4 |
| IDE | SLC25A16 | DBNDD1 | PRKRA | PCP2 | IRAK4 |
| SNAI1 | ABCA9 | ANKDD1A | PDK2 | C10orf35 | ITPKA |
| BCL6 | ASPN | SMTN | ZNF529 | GPSM3 | ZSCAN21 |
| SEC23B | ANXA7 | GH1 | FHOD1 | NO_MATCH_74 | FKBP8 |
| IFNA4 | EBAG9 | PTP4A1 | DNPEP | AVPI1 | CLK1 |
| SYTL4 | SLC22A4 | GJA3 | TGFBRAP1 | BAD | SYNE3 |
| RCBTB2 | SCLY | FAM167B | SLC43A3 | PNOC | RELA |
| FNDC7 | GPR107 | CALM1 | SERPINB10 | M1AP | TMEM204 |
| IFNA13 | IGF2BP2 | C16orf70 | NO_MATCH_251 | PRMT6 | TEX101 |
| CACFD1 | SENP2 | DCAF4 | ZNF486 | RPL32 | ZNF8 |
| IRS1 | C6orf222 | CLIC5 | C14orf180 | CCDC117 | PSMB3 |
| KLF2 | MYNN | MGAT2 | CLDN17 | ANKRD33 | AVPR2 |
| AFG1L | WIF1 | NDUFB2 | RNF5 | COLQ | FGD4 |
| OSBPL8 | BBOX1 | CT47A11 | RPL36 | RNF212 | MUCL1 |
| PIM2 | ARHGEF26 | REN | MPPE1 | PARVG | NEIL1 |
| SOX5 | WT1 | DYNC1H1 | EFCAB2 | HMGB4 | LCMT2 |
| ONECUT1 | TP73 | ZNF232 | LAX1 | YIPF6 | NBPF9 |
| MSX2 | MKRN3 | IRF9 | PKD1L2 | ADAP1 | MMS19 |
| SETBP1 | EID1 | TNIP1 | ACAA1 | MOCS3 | CD79A |
| CALHM1 | KIT | SLC5A10 | CLDN19 | SEMA4G | BLVRB |
| TOX | TMEM120A | SUV39H1 | MSL3 | GRTP1 | PLEKHF2 |
| ASAP2 | PALM2 | KIAA1147 | KLK3 | RRP8 | CDRT4 |
| GDF9 | FBXO2 | EIF4H | BZW2 | PHC2 | FHIT |
| ARHGEF2 | PTPN20 | SOWAHB | DPP10 | SMIM14 | CHRNA3 |
| RBFOX1 | SEMA4C | DCT | TRIB2 | HRH1 | PCK2 |
| SRRT | EML3 | PPP1R7 | IGSF11 | FAM76A | ARL6IP5 |
| MITD1 | FAM104A | FYTTD1 | ZNF784 | C2orf83 | GALNT9 |
| MCM3 | CPEB4 | OAZ3 | PRC1 | RCCD1 | ATF7IP |
| EGR1 | PDZK1 | KMT5B | PIN4 | CBSL | NR1I2 |
| ZCCHC24 | NDUFA5 | GGA1 | WNK1 | GRPEL2 | ARL10 |
| PRKACA | PYCR1 | ATP10D | PCMTD1 | C1orf198 | TMEM55A |
| BHLHB9 | NME5 | MAST2 | YIF1A | C1orf146 | CALML5 |
| TSC2 | TMTC4 | CERS4 | PBX3 | CFHR4 | PDK3 |
| PRR15L | SHARPIN | CD48 | HSD17B3 | LILRB4 | GTF2A1 |
| ESRRG | ZNF300 | MUC15 | COL9A3 | C1QL2 | HNRNPA2B1 |
| TOBI | DDAH1 | CAMK1G | MOB1B | SQSTM1 | DYNC1LI2 |
| PIM3 | HIST1H2BF | IFT46 | GRAP2 | UCN3 | NXPH2 |
| KLHL2 | ENTPD2 | Grid1 | SSH3 | CTNS | LOC653513 |
| RNF40 | PTGIS | BRSK1 | ZDHHC4 | RGL3 | SLC35E2 |
| ZNF140 | GOLGA2P11 | PLSCR5 | CFL1 | FXYD2 | NO_MATCH_61 |
| FBXO3 | MRPS5 | STK32B | GALR2 | TAS2R13 | NEK7 |
| RBM45 | HSD17B1 | C10orf71 | NEMP1 | VAPB | SCGB2A1 |
| ZNF345 | PDE10A | EIF3G | CAMK1 | MRPS18C | HIST3H3 |
| ZBTB21 | DNHD1 | RLN2 | PTMA | RAMP3 | PCP4L1 |
| TSPY1 | Sf3b3 | SPIN2A | XLOC_l2_007111 | CHICI | SNAPC5 |
| SLC2A4 | UGT1A6 | UCHL1 | LOC102724813 | LACTB | LHFPL3 |
| MCTP1 | TMSB4Y | PRDX5 | TMEM189 | PRSS45 | TK1 |
| BCCIP | NO_MATCH_1 | ZNF566 | SCAMPI | PDP1 | ZBTB22 |
| LMX1B | NO_MATCH_36 | VCX2 | PYM1 | TPM3 | URI1 |
| ZBTB20 | CX3CR1 | THAP11 | EFHD1 | RDH13 | C10orf88 |
| XLOC_l2_015578 | VPS39 | MS4A4A | UPB1 | DNASE2 | APP |
| FOXM1 | EXOC3L4 | PDILT | PDPN | ZCCHC10 | ANGPTL4 |
| SEC23IP | RORA | CXorf21 | SMARCD2 | DEPDC5 | PIK3R2 |
| GCM2 | PAK5 | ACTA2 | PARM1 | ENOPH1 | PAQR5 |
| ZNF224 | GNB4 | EPB42 | FLCN | NO_MATCH_238 | MAGOH |
| CD38 | SPRY4 | HSD17B10 | CD_99 | ELF4 | VSTM4 |
| EMILIN3 | HIST1H2AG | CNIH1 | FABP6 | EPHX3 | ATP5J |
| USP29 | TRH | PARP16 | ITGA4 | MAGEC3 | SNF8 |
| PWWP2B | CCND1 | RAPGEF5 | RAB11FIP2 | ADAD1 | NO_MATCH_112 |
| HEY2 | CABP4 | KPNA5 | EFCC1 | KRT18P55 | NXNL1 |
| FAM46A | PIAS3 | TMEM26 | SAMSN1 | TRNAU1AP | UTP4 |
| ZFAND3 | TRPC4 | LDLRAP1 | TRIT1 | KDELC2 | ATP5I |
| HNF4G | DNAJC1 | PSME4 | MMP13 | PIGO | PDGFD |
| GATA4 | ISY1 | RNPS1 | C2orf54 | RNF130 | CNOT7 |
| NFE2L2 | GIMAP4 | ACTG2 | SSC4D | DIRC1 | TMEM207 |
| SPDYE17 | WWTR1 | GTF2IRD2B | VAMP5 | OR1A2 | SELENOP |
| FOXA1 | DDIT3 | ABHD4 | NHP2 | TRIM27 | ATF6 |
| BFSP1 | VPS45 | TMEM229B | DAGLB | RBL1 | MIS18A |
| DES | PRRG3 | NOP56 | GLRX2 | TSPY26P | STK38 |
| UBA7 | NSG2 | SCNN1B | FAM8A1 | GSTM1 | ZCCHC17 |
| C9orf3 | GPR50 | DDX31 | AMACR | PLPPR5 | APTX |
| GLIS3 | BICRAL | PAX8 | SPPL2C | SUN3 | LITAF |
| ADD2 | C8orf34 | KLK13 | ST3GAL4 | ACSF2 | SLURP1 |
| TEX261 | SLC30A6 | CBS | TRIB1 | SLC30A2 | FDX1 |
| OCRL | CYP4F2 | CLEC4F | DENND1A | CFAP77 | AAR2 |
| BHLHE23 | XLOC_010217 | SP6 | GRIN1 | HOXB-AS3 | MAZ |
| TFDP1 | DCUN1D2 | TNFRSF11B | BPHL | REP15 | ZSCAN32 |
| GPBP1L1 | PUS10 | TSC22D4 | CSF2 | MMP28 | NUDT14 |
| MBNL1 | MFSD5 | C1QB | TMEM82 | OR2D3 | LRRC40 |
| PHF21A | ACER3 | INTS4 | CPA2 | PPWD1 | |
| KCTD1 | TMEM110 | SPSB2 | DNMT3A | TFF2 | |
| KRT23 | CALD1 | MYOD1 | KIF2C | C6 | |
| MORF4L2 | NO_MATCH_98 | DNAJA2 | MTAP | PCDH20 | |
| DTL | GLO1 | WDFY2 | NO_MATCH_58 | C1QL4 | |
| RHAG | VTI1B | ASRGL1 | CD4 | EEF1B2 | |
| PLG | PRDX4 | KCNA7 | BBS5 | CCDC114 | |
| SMG5 | UNC45B | DAZ2 | APEX1 | NUDCD3 | |
| PHKA1 | PSEN2 | FAM217A | DYNC1I1 | GPD1 | |
| NR1D1 | TMEM192 | MNAT1 | LRRC27 | ALG8 | |
| DARS2 | FOXP3 | KIR2DS1 | AGMAT | TCTN1 | |
| NOVA1 | HSP90B1 | NCKIPSD | NBPF15 | IQCH | |
| CCDC13 | SPAG9 | ZNF85 | LMBR1L | PIEZO1 | |
| ERF | NSUN2 | SPEM1 | PDZD4 | INTS4P1 | |
| IFNA1 | GYS1 | SLC13A5 | TMEM45A | PIK3R5 | |
| ZNF569 | CST6 | HEY1 | CD72 | COX18 | |
| MBNL2 | ASCL2 | RASSF8 | NEUROD6 | PTPN5 | |
| SGF29 | GSE1 | C7orf26 | YAP1 | PPIL6 | |
| KNG1 | POTEB3 | CARD11 | IFNAR2 | TSNAXIP1 | |
| SORT1 | MFSD4A | TRHDE | MYBPH | SULT1A3 | |
| TCF7L2 | SLC17A1 | EFNA5 | KCNK15 | ZFP41 | |
| MAGEB6 | KYNU | KPNA6 | C1orf127 | D2HGDH | |
| GZMA | KLC1 | NAPB | EDN3 | SFXN2 | |
| TGIF2 | CYP39A1 | UCHL5 | HSPD1 | KRTAP1-1 | |
| PNRC2 | FAM199X | ZNF512 | XG | SMARCD3 | |
| EXOC2 | GLIPR1L2 | SCNN1G | COX6A1 | OGFOD3 | |
| DCAF12L2 | KIF7 | KCNK5 | GNG7 | C1orf162 | |
| HOXC9 | CNTN4 | C1orf21 | RASGRP3 | ARMCX2 | |
| FBXL13 | ZNF684 | ZC3H7A | N1F3 | PSKH1 | |
| EBF3 | NO_MATCH_182 | WDR53 | MAD2L1 | PRDM5 | |
| RIBC1 | ZNF98 | OR11A1 | EPHX1 | SPEF1 | |
| FOXO3 | SETD5 | ERMN | PRPS2 | ZNF783 | |
| KDR | SLC4A1 | CPA1 | PPP1R16A | ATP5E | |
| APOBEC3B | MS4A10 | SCAMP4 | IMPA1 | TGFBR3 | |
| ZNF12 | LNPK | HVCN1 | CAMK2B | NPAS1 | |
| SMARCAD1 | ADD1 | CTGF | DYNLL1 | FBXO28 | |
| ASB16 | TIGD7 | GPR63 | CXorf58 | EML4 | |
| UBE3B | SHOX2 | FANCD2 | TMEM155 | C12orf50 | |
| NRP1 | NPR3 | VIT | UBE2S | DCLRE1A | |
| PCDHGA2 | VGLL4 | TIYH3 | ZNF3 | BPIFA1 | |
| PCDHGA5 | HSCB | WNT9B | YIPF1 | SLC9A8 | |
| HNRNPF | SLC25A43 | CFAP57 | METTL7A | B3GNT9 | |
| JSRP1 | RPE65 | TSPAN8 | CFAP36 | SCG5 | |
| MTA1 | PCDHAC1 | LRRC39 | SOX10 | CAPN3 | |
| MPPED1 | PIGA | VDR | AGER | CCL7 | |
| AMPD3 | AUP1 | KRT6A | RAB8B | FUK | |
| RNF11 | RBPJ | GNL1 | PARPBP | TBC1D28 | |
| DRG2 | EEF1A1 | TNFRSF10A | TGFB3 | CMA1 | |
| LEF1 | GPR146 | LIG4 | ADRA2B | TRMT6 | |
| FAM3C | C3orf62 | GMFB | EOGT | LYN | |
| PIGR | JAGN1 | DAP3 | ADSSL1 | CAMKMT | |
| MRRF | COROIC | MRPL30 | DPM1 | NEK2 | |
| FAR1 | UXS1 | TYRO3P | SPOCK3 | VEGFB | |
| OXR1 | ANKZF1 | PDE4D | DYDC2 | EEF1G | |
| FOXJ2 | TMEM134 | RNF214 | TAPBPL | SETD3 | |
| ASIC1 | ZNF207 | GPR180 | TMEM255A | PLD4 | |
| PDE4B | CCND3 | SLC39A9 | CWC22 | KLHDC4 | |
| SCAP | NOP16 | RAB3A | ACMSD | TEX45 | |
| SP1 | C11orf24 | ZNF625 | P3H3 | ANKAR | |
| CDON | AP2A2 | NTN5 | A4GALT | ICAM4 | |
| SLC41A1 | TTLL2 | BRINP3 | HIKESHI | CYB561 | |
| MRGPRX1 | PPIP5K1 | ZBTB49 | RIPPLY1 | MND1 | |
| ERCC5 | PHLDB3 | CXCL14 | TOR1AIP1 | LELP1 | |
| TNFRSF19 | H2AFY | MPG | ASB6 | KRT8 | |
| MRPL37 | NDEL1 | TSPAN31 | MARVELD3 | IL3 | |
| FAM151A | METTL25 | LSM10 | HEBP2 | VRK2 | |
| SSRP1 | EYS | GPR15 | CAPNS2 | OR13J1 | |
| DRD4 | TF | OR4K2 | PDE6D | CA11 | |
| USP26 | MS4A7 | SERPINA4 | PBX4 | MNS1 | |
| ARR3 | ELOVL5 | ZNF816- | FASLG | TRIM48 | |
| | | ZNF321P | | | |
| MITF | ZNF816 | CHRD | ASPRV1 | TRIP6 | |
| JOSD2 | METTL13 | ACTL6A | REG3G | CORO2A | |
| HS3ST3B1 | CFDP1 | RGS8 | STXBP3 | PTPN12 | |
| FAM160B1 | KRBA1 | FES | AMY2A | COX8C | |
| SLC25A42 | PTGDR2 | RFPL4B | TMPRSS3 | OR8D1 | |
| ZIM2 | JUP | CEP162 | HAND2 | SCLT1 | |
| PCDHA6 | SLC35A3 | CDC42EP3 | DYNC1LI1 | STK3 | |
| FAM20B | SLC35B1 | ZNF474 | ADGRG2 | DUSP26 | |
| PDE9A | CUTA | FBXO25 | CXXC1 | NO_MATCH_155 | |
| JKAMP | PCID2 | AGBL2 | AAAS | WDK62 | |
| CTDSP1 | SF3B4 | HAO1 | ZNF202 | IL1A | |
| NRSN2 | CLCC1 | PHAX | EIF3J | CABP5 | |
| BAZ2B | COQ7 | RASSF3 | DLK2 | FBL | |
| PSG9 | RPS6KL1 | GIPC2 | FRMPD2 | ZP1 | |
| SS18L1 | RAB23 | POLR2L | C14orf39 | RBM46 | |
| HRAS | FAM161B | CDHR4 | ARHGAP45 | HDAC3 | |
| Prkd2 | ALOX15B | ASH2L | PPCDC | APEH | |
| PSMD4 | LLGL2 | CDK17 | CETP | NO_MATCH_133 | |
| AKT1 | OXA1L | BNIP3L | B9D2 | TNFAIP3 | |
| IRX6 | PLCL1 | TMTC1 | EVA1C | TBC1D20 | |
| MEPE | IL17B | CREB3L1 | ATP1A4 | ITM2C | |
| ARAP1 | LONP2 | MYH7 | SLC35D1 | TMEFF1 | |
| MAPK8IP3 | ECHDC2 | EFNB2 | TXNL4B | UBE2Z | |
| KSR1 | SUPT4H1 | LRRIQ1 | KIF3A | ADGRD1 | |
| DRICH1 | HAUS7 | FAM219A | ERP27 | KIAA1644 | |
| SPOPL | FURIN | HID1 | NO_MATCH_239 | CRAT | |
| ZNF503 | WDR48 | MIR7-3HG | ARMC8 | GALNT16 | |
| RARA | CDC42EP4 | PTBP1 | KRT80 | FIGN | |
| SPON1 | FAM103A1 | CIPC | PCBD1 | AGA | |
| TRIM37 | SLCO1B1 | FLT3 | SEMA4F | C11orf70 | |
| TNFRSF13B | KBTBD7 | SLC23A3 | NO_MATCH_124 | STAC3 | |
| MUC20 | SURF4 | RPS3A | GALE | G2E3 | |
| TMPRSS13 | GCC1 | FBXO39 | IL5RA | TUBA1A | |
| C1orf228 | AHCYL1 | DPYS | HIST1H3A | CAB39 | |
| DTX3L | RFX5 | IDS | PRB3 | PRR4 | |
| MMP19 | MAJIN | HOXA5 | LOC102724428 | DNASE2B | |
| MSH2 | GGA2 | POLM | NO_MATCH_89 | RAB40A | |
| TPST1 | RABGGTA | CRIPT | ZWILCH | CREG2 | |
| TEC | CCDC137 | KCTD14 | NO_MATCH_194 | NOSIP | |
| BORCS8-MEF2B | HOXA3 | LIN9 | BAX | FCRL1 | |
| SYBU | TCERG1L | TPPP | RNF13 | F2RL2 | |
| BTBD18 | ADPRHL1 | RNMT | CLEC14A | GREM2 | |
| PPP2R5D | SLC20A2 | PMM1 | KCNRG | DLEU7 | |
| TLK1 | BRD4 | HSD17B11 | TMEM45B | NO_MATCH_187 | |
| ZNF219 | TMEM205 | CASC10 | OLR1 | PITPNM3 | |
| ZNF311 | SHC4 | SDCBP2 | GLP2R | EIF4A3 | |
| CTBP1 | DEK | SH3BP4 | RAB3B | TXN | |
| IKZF5 | Med1 | SNRPD2P2 | KDELR3 | SLC5A8 | |
| POLA2 | GABBR1 | NR2C1 | BCL2L11 | TBC1D13 | |
| ARIH1 | FOXJ1 | ZNF134 | HCK | PRDM7 | |
| CEP135 | TP53BP2 | TSPAN12 | ERH | MGME1 | |
| DGKK | SH3BP5 | RPS6KA3 | PBK | LRRC74A | |
| FGFR2 | FBXO5 | AQP11 | RAB2B | TVP23A | |
| PACSIN2 | BRMS1L | FNDC3B | NO_MATCH_144 | NO_MATCH_7 | |
| TRMT5 | DPPA3 | LAMP3 | BMT2 | GPN3 | |
| STMN2 | OSBPL3 | GDF15 | DMP1 | TVP23C | |
| CYP4B1 | TLDC1 | TMEM43 | TNS4 | SHOC2 | |
| ZNF669 | ITGB8 | ODF3 | PANK1 | NO_MATCH_120 | |
| RELB | LINC01105 | MCUR1 | SYCE1 | WARS2 | |
| ZNF572 | PIP4K2A | EGFR | FECH | QRFPR | |
| PDGFRB | CPT1B | ZPBP | PAK4 | IMP3 | |
| C9orf170 | GNAS | XLOC_l2_015600 | CLEC1A | SNAPC2 | |
| LCA5L | CRY1 | NO_MATCH_67 | LILRA4 | NO_MATCH_138 | |
| GSC | ZNF107 | SLC12A8 | FUNDC1 | UBE2DNL | |
| EZH1 | NQO2 | FAM189A1 | ABHD18 | PI15 | |
| OR13C3 | SERPINA12 | XCR1 | MCAT | BAAT | |
| CLCN6 | TAMM41 | DUSP6 | AP3M1 | ZNRF2P2 | |
| ANGEL2 | LIN7A | ZDHHC11 | IDH3B | EXOC3-AS1 | |
| ATF4 | GNB1 | NR2F1 | HSPBAP1 | MVK | |
| VKORC1 | B4GALT5 | WDR83 | EPHA3 | C10orf25 | |
| SYTL3 | IL20 | PKD2 | SLA2 | IFI30 | |
| PDIA5 | TAT | EMC6 | KIAA1324 | TM4SF18 | |
| MAGEE1 | HHAT | GDPD3 | GATAD1 | B4GAT1 | |
| SYT4 | DYRK3 | PDZD11 | ATG3 | PPIB | |
| BATF | ATF5 | SLC2A13 | SLC51B | PCCB | |
| TCF7 | REM2 | GCAT | EYA1 | EGFEM1P | |
| TONSL | ITGB7 | IRX2 | BLZF1 | SPOCK1 | |
| COPB1 | RNASE4 | RFWD2 | SURF1 | XPA | |
| PPP2R2D | TEKT5 | TMC03 | CRYM | RPL27A | |
| NLRX1 | FBXL14 | MYO10 | MRPL28 | ZNF483 | |
| RAI1 | GALNT8 | EHMT2 | KCNE3 | ECSIT | |
| MSN | AKAP14 | MCL1 | C21orf2 | DIO3 | |
| LUC7L2 | ZC3HAV1 | NAA40 | BATF3 | HLA-DMA | |
| TBC1D2B | MRE11 | IL17RD | MLLT6 | RAB6A | |
| RFX4 | ANKRD1 | CCR2 | KCNJ11 | MS4A6A | |
| SLA | FSCN3 | CEP19 | HIST1H2AC | UQCR10 | |
| ENTPD5 | C1orf50 | PILRB | RNF175 | NATD1 | |
| FAM19A4 | RNF133 | C3orf38 | Mok | MRGPRE | |
| DIP2C | FZD1 | ZCRB1 | CACNG3 | CSNK1A1L | |
| TFDP2 | ARID5A | WDR1 | DACH1 | MTCH2 | |
| FAM126A | DMBT1 | GRIN3A | TMED5 | ADTRP | |
| MAFB | ACSM3 | HIST1H2BC | FABP5 | CD7 | |
| LRWD1 | ISLR2 | MYLK4 | CCDC70 | SYT17 | |
| ACSL3 | ADGRG5 | SUGP1 | MYOZ2 | PRELID2 | |
| CFB | ARL6 | PNKP | PNLIP | GOT1L1 | |
| IRF5 | RPS6 | SLC6A20 | CCNI | TSSK2 | |
| Myt1 | DAZAP1 | MRPS22 | RDH12 | KRT36 | |
| EPB41L1 | ROCK2 | USP47 | SH3BP5L | PA2G4 | |
| TPD52L1 | MIXL1 | TCEAL8 | CHMP1B | CDC42BPG | |
| PAX7 | ANXA6 | SCD | PCDHB10 | ALG13 | |
| CCDC32 | TGOLN2 | PTDSS1 | AIF1 | FER1L6-AS1 | |
| UCKL1 | AP3D1 | SRMS | TTC4 | EID2B | |
| FUT1 | HIST2H2BE | ACVR1 | LCE3C | DGCR8 | |
| LEPROTL1 | DDI1 | LDB3 | SMIM11A | TMEM97 | |
| GLYR1 | DR1 | PPIL2 | NR2C2AP | ASB8 | |
| ELAVL4 | DPM3 | IL23R | AGO3 | CARD6 | |
| PDE3B | ELF2 | FOXB1 | OR4F15 | SNX31 | |
| XPR1 | HIST1H1C | KPNA2 | GPR45 | OCM | |
| UBALD1 | C1orf115 | LGALS12 | C9orf85 | TTC9C | |
| PTGES3L- | CDC25C | CPPED1 | MKRN2 | CCM2 | |
| AARSD1 | | | | | |
| LRCH2 | GIMAP5 | TSPAN7 | MLYCD | AIM2 | |
| ST5 | EAPP | RTFDC1 | SMCO1 | NAA16 | |
| YAF2 | LOXL4 | SH2D1B | TATDN1 | CEP170B | |
| OTX2 | MYLIP | OGDHL | PPARD | CTPS2 | |
| SERPINH1 | RFTN1 | MRPL27 | VSIG2 | ACOT13 | |
| SIK2 | ZMAT1 | ETNK1 | ACOT7 | PLA2G4C | |
| ZBTB26 | AP3S1 | ZNF341 | CNDP2 | BSCL2 | |
| VENTX | HNRNPH1 | DAPK2 | GBGT1 | ZNF266 | |
| CLIP3 | DPP8 | RAB20 | TRIM41 | CMTM8 | |
| SIRPA | NFKBIA | ITPK1 | APOL1 | RAC3 | |
| OLIG3 | RASGRP1 | KRTAP13-1 | ACTBL2 | FSD1L | |
| PKD2L1 | CCDC42 | MRPL45 | NEK10 | PCNX4 | |
| TNFRSF1A | NOX1 | B3GNT6 | Tmprss7 | AP4E1 | |
| HOXB7 | GTF2H1 | XYLB | ZNF34 | FNDC1 | |
| POU2AF1 | ERP44 | FNDC11 | INTS12 | KIF14 | |
| TCF7L1 | MLH1 | C11orf54 | AP2M1 | ADCY9 | |
| CTDSP2 | UST | LRRC31 | ZNF189 | ELF3 | |
| ROGDI | ADCK1 | KCNK2 | SSR2 | ROPN1B | |
| MADD | TMEM63A | NO_MATCH_134 | KCNE2 | DHX57 | |
| ANKRD20A3 | SMARCE1 | MEOX2 | LHB | ATP6V0E2 | |
| FAM19A1 | GPR143 | NUBP2 | CAPSL | TPPP3 | |
| XBP1 | KIAA1586 | FANCM | ZNF770 | GALNT4 | |
| SGSH | TSR3 | SPIN2B | TGFBR1 | TMEM234 | |
| TM9SF1 | PSMA1 | CNST | RCN1 | ZNRF1 | |
| AXL | RAD23A | NECAB2 | GALNT14 | PFDN2 | |
| BRD2 | ZNF571 | PRR14L | ST8SIA2 | NO_MATCH_240 | |
| MFAP4 | HSPB7 | TXNDC12 | NO_MATCH_151 | CASP7 | |
| C10orf67 | PGAP2 | C1orf220 | PARP11 | PGD | |
| POGK | RHBDL1 | PCYT2 | N6AMT1 | RAB32 | |
| JAG1 | NUDT16L1 | FOXN3-AS2 | TMEM257 | RBFOX2 | |
| DOLK | SLC44A3 | SLC47A1 | NO_MATCH_85 | TPD52L3 | |
| NR2E1 | TOX2 | C8orf74 | DEFB134 | LHFPL5 | |
| GCM1 | MRPL12 | C1QTNF1 | LINC01657 | ZBED8 | |
| WDR41 | TSP_ANU | IL7 | PCMT1 | SLC41A3 | |
| PKN1 | HNRNPK | DUSP15 | KRT71 | TICAM1 | |
| HOXA9 | ADGRA2 | LIMCH1 | YWHAB | SNX9 | |
| UBE4A | TMEM123 | ZDHHC1 | ZNF253 | G6PD | |
| C6orf118 | CYSRT1 | CPZ | METTL21A | LMO3 | |
| CCNB1IP1 | COPG1 | C17orf97 | LRTM2 | DUT | |
| CT45A10 | ZNF559 | ABO | RPS12 | APLF | |
| HS3ST5 | CKAP5 | KIF25 | RPS27L | LOC100289561 | |
| IKZF3 | TPRA1 | NO_MATCH_87 | TAOK3 | GSDMB | |
| CYP3A4 | GPM6B | IHAP6 | TSR2 | CEP170P1 | |
| FMR1NB | TYSND1 | ZBTB38 | DNAJC6 | CFHR5 | |
| ACADS | CWF19L1 | ASCL4 | DDX19A | OTUD6A | |
| GALNT2 | FAM71E1 | SEMA6D | SENP8 | SNRPE | |
| CKAP4 | CCDC138 | ZNF263 | NOL6 | FARSB | |
| WNK4 | FAM46B | ADORA2B | XLOC_l2_006955 | DPY19L2 | |
| PPP1R12A | 3-Sep | SAR1A | KRTAP22-1 | TTC8 | |
| CAP1 | SGK3 | SLC50A1 | DDX21 | LRFN4 | |
| C1orf210 | L3MBTL1 | NO_MATCH_60 | TEX28 | MMAB | |
| DLK1 | LSAMP | CASP14 | UROD | MS4A1 | |
| NCS1 | VPS4A | C21orf62-AS1 | SPIN3 | LRRC46 | |
| IGF1R | LRRTM4 | SPACA4 | C15orf40 | DUSP7 | |
| UBLCP1 | TET2 | SSX5 | FSHR | UFSP1 | |
| FAM212A | HADHB | B4GALT2 | LMAN2L | RIOK3 | |
| DPYSL5 | RHBDD3 | HNRNPA0 | ARG2 | PCYT1B | |
| NKX2-3 | CYP1A1 | NO_MATCH_167 | STAR | OR5M8 | |
| ZNF680 | TIMM22 | NUDT8 | ARF6 | NR3C1 | |
| MMRN1 | PDE1B | CAPZA2 | ERBB3 | NO_MATCH_49 | |
| JADE3 | ZNF169 | NSUN4 | ARL13A | ZNF526 | |
| RAD17 | UBE2I | PAGE5 | FGFR1OP2 | REEP3 | |
| CAPN9 | MOGS | TEX264 | MAATS1 | AHDC1 | |
| PRRT3 | ATG16L2 | PIPSL | NAALADL2 | TKTL1 | |
| GPC1 | BRPF1 | LOC403312 | TPM1 | SLC39A7 | |
| PHF8 | GALNT1 | PLA2G2D | TMEM8B | NXPE1 | |
| BANK1 | AKT2 | C11orf98 | SRD5A2 | ACCS | |
| SMARCAL1 | PSMG1 | PPP3R2 | IZUMO1 | CMTM2 | |
| SPSB4 | ZBED1 | ROPN1L | TMEM231 | PRAME | |
| COX7C | TMCO2 | PJA2 | WSCD1 | RNF216 | |
| E2F8 | NO_MATCH_14 | JUNB | FGF12 | TTBK2 | |
| TRIM16 | ERICH5 | ACSS3 | ECHDC1 | XLOC_011297 | |
| PELO | OR52N5 | CYLC2 | DEFB106A | WDK20 | |
| RABGGTB | DCAF4L2 | BCL2L1 | MPDU1 | TMEM255B | |
| DSG4 | VGLL2 | ABCF3 | TMEM236 | PDCD10 | |
| ZNF791 | SLC35C2 | OR3A4P | ITLN2 | SPATA5L1 | |
| HOXD4 | STMN3 | RAB39A | TIMM8A | STK38L | |
| TMED4 | PPP3CC | FANCE | XLOC_013491 | MYADM | |
| SARDH | OR56B1 | LRRC45 | PLOD1 | SLC1A7 | |
| RPGRIP1 | TMBIM4 | ANKRD12 | OR51B4 | MLKL | |
| HPS1 | HOXD3 | MYRIP | BRWD1 | RAB31 | |
| TRAPPC8 | TSPAN10 | CCDC181 | TMEM30B | DDHD1 | |
| TMEM179B | GSPT2 | RPL4 | GTDC1 | GDI2 | |
| LRRC49 | FAM222B | PCYOX1 | RHNO1 | SLITRK4 | |
| ACSBG1 | C19orf38 | RGS18 | RNASEH2B | ORMDL1 | |
| GBP1 | LCORL | UGT1A1 | C16orf78 | KRTAP9-2 | |
| ZBTB48 | YY1AP1 | NO_MATCH_268 | REPS1 | RAB1B | |
| CCER1 | MRPS6 | WDR89 | MLX | FBXO44 | |
| ZDHHC23 | PSTPIP2 | ARPC5 | TTC26 | MAPK8 | |
| PGAM2 | AFP | OSBPL5 | STK35 | SYP | |
| CD83 | DZIP1 | LSM12 | SAV1 | HLA-L | |
| FAM46C | FAM219B | PDK4 | AAMDC | SOD2 | |
| ZZZ3 | TMOD1 | RPP38 | RP2 | IMEM164 | |
| DENND6B | FGF11 | LOC399886 | SUMO1 | TNFRSF21 | |
| KCNH7 | ZNF628 | MGST2 | FNTB | UPK2 | |
| CPEB1 | RHOH | CATSPERD | CHMP2A | ALG12 | |
| KIAA1524 | MAP3K2 | IMEM260 | OSBPL2 | BFAR | |
| HJURP | CHMP7 | SPHK1 | HAUS1 | SLC23A2 | |
| MRPL15 | AP3M2 | PAIP2 | FAHD1 | VEGFC | |
| FEZF1 | FANK1 | LGALS3BP | PPIL1 | MPP5 | |
| TAS2R4 | ESYT1 | PPP1CC | VCAM1 | CCDC121 | |
| ZNF366 | RAB3IP | GPR89A | CFI | DPAGT1 | |
| PHACTR3 | ZNF561 | BFSP2 | RARS | TSG101 | |
| APOE | EGFL6 | CCT4 | APBB1 | NT5C1B | |
| OR6K6 | PKIG | MPZ | SLC18A2 | RTN2 | |
| HIST1H2BG | MYCBP | CCL22 | EFS | PDGFRL | |
| RNF8 | RASGRP4 | SLC25A33 | PPM1K | CAMTA2 | |
| ZNF618 | NELFCD | TPP1 | GRP | APCS | |
| IFNL1 | RPS8 | RPAIN | RORB | TBC1D7 | |
| BABAM1 | PHLDB1 | NR2E3 | PRICKLE3 | CAMP | |
| SPATA6 | XLOC_l2_006624 | NO_MATCH_169 | ARL11 | PLIN2 | |
| TMED2 | PTK2 | GALK2 | CYP27C1 | CALCOCO2 | |
| MEP1A | ZYX | POLR2M | PTP4A3 | GTPBP8 | |
| XLOC_005244 | TCEANC2 | CKAP2 | CNTN2 | TRIM17 | |
| FEZ1 | BANF1 | CDKN3 | PRPF18 | UBTD2 | |
| CRYL1 | VIRMA | CCDC97 | AQP7 | CLINT1 | |
| GPR183 | GRHL1 | UBE2M | HIST1H2BA | SHPK | |
| TMPRSS2 | OR52W1 | OGT | SYNJ2BP | MRPS35 | |
| EPYC | AK9 | CCDC74B | BORCS8 | ZNF394 | |
| DNTTIP2 | CDCP1 | ADAM22 | SRPK2 | PLSCR3 | |
| PPP3R1 | CTRC | ADCK5 | OSTM1 | PRPS1L1 | |
| MECP2 | ST6GAL1 | TPD52L2 | MYOC | CAMK2D | |
| PGAP3 | C15orf48 | RBBP5 | C6orf201 | CYB5R1 | |
| MPC2 | ATP1B2 | EIF2AK1 | BRK1 | PNMA1 | |
| ZNF250 | PEX11B | LAMP2 | CYB5R2 | CBFA2T2 | |
| TXNDC5 | SCHIP1 | SSUH2 | PCSK9 | GLB1 | |
| PIK3CA | ERCC2 | ORM1 | PPIA | PLP2 | |
| LOC100287896 | SRSF10 | PINX1 | OPRM1 | TSHB | |
| NBN | NO_MATCH_31 | DCAF11 | BCAS2 | CTLA4 | |
| NANOS3 | SLC46A3 | FGL1 | EIF2S1 | GPR173 | |
| NO_MATCH_52 | R3HCC1L | SMN2 | SPACA3 | CHRNA1 | |
| MSANTD3 | PRKX | RILPL2 | NO_MATCH_101 | UPP1 | |
| MADCAM1 | POLR3GL | FH | CHIT1 | LRRC6 | |
| DUSP22 | TGM1 | MAPK12 | MSC | CHI3L1 | |
| ZC3H11A | KCTD12 | KIF5C | BCAS1 | SBNO1 | |
| KIF24 | TLE3 | GK5 | EIF2S2 | CEACAM3 | |
| SNCAIP | STT3A | RAP1GDS1 | TNFRSF17 | AFTPH | |
| FGFR1 | RND1 | DCLK3 | NEFL | BAIAP2-AS1 | |
| KIRREL1 | UBASH3B | SYT6 | MS4A12 | RASEF | |
| GTF3C5 | EDEM3 | ACKR4 | DIAPH1 | ASPA | |
| DGCR2 | GABRP | POT1 | MST1R | CHRNB4 | |
| PPFIA1 | VEPH1 | PLSCR4 | SLAIN1 | DNAJC27 | |
| BRPF3 | LAMTOR3 | LSM14B | NXF5 | RPS26 | |
| CRAMP1 | CWC27 | GPD2 | SLU7 | USP48 | |
| PAIP1 | KRAS | SLC9A3R1 | TOMI | DNASE1L1 | |
| NACC1 | POP7 | CBARP | BCL7C | DAPK3 | |
| KLHL40 | CDK9 | SLC26A11 | PUDP | ST6GALNAC6 | |
| TMEM246 | ACAD11 | TUSC3 | PIGH | CDHR2 | |
| KCNA6 | TBPL2 | ALDH3A1 | CHRDL1 | MICAL2 | |
| XKR3 | DNAJC11 | ATG13 | SUPT5H | PRAG1 | |
| ARHGAP8 | CCDC68 | MZT1 | ASB9 | ZNF273 | |
| GPR62 | BPESC1 | UBE2A | CEP55 | HERC3 | |
| MAST3 | GBP4 | LIMK1 | UMPS | SLC38A11 | |
| JAKMIP2 | GDAP1 | TERF1 | ZNF75D | RNASEL | |
| SLC38A7 | IFT81 | F3 | OR6A2 | CREB5 | |
| ZFP14 | IFI27 | AXDND1 | ZNF785 | MLN | |
| RGSL1 | CHMP4A | SIL1 | SLC12A1 | RNF141 | |
| CRX | PAPSS2 | KDELC1 | BPIFC | RPS27A | |
| TCF25 | TFAP4 | RPS19BP1 | RAB39B | LPAR3 | |
| OTX1 | PMEL | NHLRC1 | PCDHGB3 | OLAH | |
| NRP2 | ARSG | Spcs2 | PHTF1 | ZCCHC14 | |
| FEM1C | TACSTD2 | GPNMB | GFOD2 | SNX11 | |
| APLP1 | ZCCHC9 | SHBG | HSPA9 | TMEM30A | |
| RSRC2 | PLCG2 | TMEM243 | ATP23 | DUSP21 | |
| CBLB | PPP1R32 | IL10 | ARMCX6 | CARS | |
| ZNF778 | DCLRE1C | DEFB105A | PRKAR2B | ZNF300P1 | |
| DIRAS3 | APOBEC3C | TRIM52 | ORAI1 | NDUFS7 | |
| ABI3 | ZBTB45 | PDE6G | DMRTC2 | GCK | |
| PSMA4 | TP63 | DRAM1 | NHLRC3 | DLL4 | |
| RRM1 | LINC01555 | NMNAT1 | MCFD2 | FAM114A2 | |
| NO_MATCH_256 | PRPF6 | LINC01465 | GABRG2 | GNG11 | |
| DDOST | TRAIP | FSTL4 | KLHL18 | PDLIM2 | |
| TGIF2LX | VANGL2 | ZFP3 | WDR63 | SOCS6 | |
| TMEM132A | PRPH | PYHIN1 | THOC1 | TMEM54 | |
| ZIC3 | LRRTM3 | RIMKLA | WDK47 | NO_MATCH_69 | |
| HOXD10 | PPM1N | SPINK6 | ARHGEF28 | IL26 | |
| MEIS2 | ADAMTSL1 | NO_MATCH_237 | ASB17 | EDA | |
| UGT1A9 | C2 | PDCL | HSD17B12 | LCN1 | |
| KCNJ1 | STOML3 | MRPS7 | ASMTL | CPLX4 | |
| IFI16 | NUMBL | CASS4 | RAET1E | KRTAP12-1 | |
| GTF3C3 | PIH1D3 | GPRC5A | SCIMP | CDO1 | |
| VN1R2 | PCBP1 | HOXA1 | C9orf43 | KCNJ12 | |
| GORAB | TMPRSS5 | ZNF638 | RTCA | VIP | |
| NTS | IGF2BP3 | EIF4B | SLC47A2 | NO_MATCH_160 | |
| MRPL39 | LRIT3 | KIF2B | HIST1H2AI | SCCPDH | |
| CDC23 | MAPT | BOD1 | NEDD8 | NO_MATCH_6 | |
| HSP90AA1 | RNF7 | Mbd5 | CLDN20 | ADSS | |
| CANT1 | NO_MATCH_220 | INSL4 | IRAK3 | FAM92B | |
| DRAXIN | TICAM2 | C2orf66 | FCHSD2 | SLAMF9 | |
| DENND2D | NDUFA7 | ANKRD29 | MBOAT2 | GRB14 | |
| NO_MATCH_11 | LOC102724984 | FLOT1 | DSTN | CCR9 | |
| CASD1 | GPATCH2L | LRRN1 | ANAPC13 | APOBR | |
| SUGP2 | SLC4A5 | ERO1A | SH2D1A | NO_MATCH_223 | |
| SPECC1L | PSMD5 | GAS2L3 | PRKCI | TRIM31 | |
| CHAT | RINT1 | IFITM2 | GNPNAT1 | IL4 | |
| TMEM185B | CDC42SE1 | KIZ | PMEPA1 | COG5 | |
| WARS2-IT1 | RSPO1 | NSA2 | C3orf30 | TCTN3 | |
| HNRNPH2 | PRR14 | COPS2 | EYA2 | OSGIN1 | |
| FSTL1 | FOXN4 | PEX19 | NCOA1 | FER | |
| BDNF | ARHGAP32 | IL37 | ACTRT3 | C3orf49 | |
| UBE3A | HOMER3 | GABRA4 | ZBTB8A | SH3GLB1 | |
| TLE6 | DPH6 | ATP6V0D1 | ITLN1 | LCP2 | |
| GLDC | ZBTB42 | MC3R | CDIPT | PIGM | |
| TCF4 | NCALD | CD3E | GOSR2 | ATP6V1D | |
| CENPQ | L1TD1 | TTC23L | C16orf54 | PRKCA | |
| SLC9A6 | OLFM3 | AFF4 | CBLN2 | LOC90768 | |
| EMP1 | ZNF428 | ABHD14B | SECTM1 | STAT1 | |
| GPR108 | CCR3 | Hoxa10 | DFFB | HSDL2 | |
| LINC00852 | LNPEP | App | TRAPPC5 | RNF168 | |
| GMDS | ATP13A1 | PRKAR1A | ATG14 | IRAK2 | |
| RPA4 | RHOA | TUBGCP3 | PDE4DIP | USP46 | |
| ALKBH7 | SLC14A1 | ZP2 | MYL12B | BANP | |
| STX4 | GUCY1B3 | GRM1 | CSRP1 | EREG | |
| OR7C1 | UBR2 | NO_MATCH_179 | ULK3 | RSL24D1 | |
| SLC52A3 | DCBLD2 | PPP1R12C | BBS10 | CIDEC | |
| IFNAR1 | SNAP29 | DCTN4 | LARP1B | MAPK15 | |
| DBF4 | OCLM | KCTD19 | TLDC2 | FAM110D | |
| BBS1 | TRAPPC10 | NKIRAS2 | POF1B | ATG10 | |
| ARL8A | XLOC_l2_000791 | NO_MATCH_77 | ARHGAP36 | CYCS | |
| POLR3E | CYB5B | NUDT9P1 | ARHGEF38 | MTPAP | |
| TTPAL | MARCO | PTGER4 | PIK3C3 | SLC39A4 | |
| WAPL | CD9 | CRKL | TLR2 | BCAT2 | |
| RER1 | RHOT1 | B9D1 | RPL13 | PEA15 | |
| NIPAL1 | STOB1 | FKBP7 | CCDC126 | SYN3 | |
| DHTKD1 | PDP2 | ZMIZ2 | BTBD1 | SLC25A32 | |
| ALDH18A1 | MLST8 | DEFB1 | NO_MATCH_197 | LCA5 | |
| GRIN2A | TFE3 | Rbp1 | CHCHD5 | FBXL8 | |
| STX17 | OSER1 | TUBA1B | FGF5 | PRR13 | |
| NTRK3 | FOXRED2 | COMMD1 | PVRIG | INTU | |
| NENF | P3H4 | LGR5 | HIST1H3G | FCRL5 | |
| MFF | Lztrl | DCTN2 | GET4 | PMP22 | |
| ZNF613 | CCDC130 | NADK | ZNF556 | DLGAP1-AS1 | |
| ZNF678 | NEUROD2 | CALR3 | RETREG3 | FERMT1 | |
| LTV1 | BMX | NMRK1 | SPDL1 | FCRL2 | |
| PLLP | MAP3K3 | SENP1 | PRIM1 | IDO2 | |
| SH3D19 | MYCBPAP | DHX38 | NAA50 | IWS1 | |
| NO_MATCH_196 | RTL8A | EHD2 | C20orf173 | BCL6B | |
| ZSCAN9 | XLOC_l2_005978 | SRSF7 | DAZ1 | NAALAD2 | |
| XXYLT1 | ZFP82 | DNAJB2 | SDR9C7 | NCCRP1 | |
| RAP1A | NPEPPS | CACNB3 | TFAM | PFKM | |
| RSPH4A | BLOC1S2 | ZIK1 | UGGT2 | PGLS | |
| IRAK1 | EPHA2 | ZMYM1 | F11R | FKBP14 | |
| ADH6 | TMEM170A | ASZ1 | MMP23B | SYTL2 | |
| RIT2 | TMEM79 | WASF1 | PIH1D2 | ALOX5 | |
| SERTAD4 | CALCRL | LOC107983965 | NUMB | LAMP5 | |
| ZNF577 | BID | HIST1H2AM | ENKUR | DEFB118 | |
| PCDHGA9 | DOCK5 | SLC6A2 | SLCO1C1 | ZMYM5 | |
| ARPC4-TTLL3 | KCMF1 | YPEL3 | RUNDC1 | IL1RAPL2 | |
| MAP2 | TAS2R19 | GFPT2 | OGFOD2 | SRPK1 | |
| POU5F2 | C3orf56 | PARVA | STX7 | RPL23AP7 | |
| NUFIP2 | PPP2CA | ACAP3 | RHOC | CCNJL | |
| CLEC18A | AGTRAP | PCDHA11 | SPRR1B | MAT2B | |
| SLC4A1AP | DFNB59 | PROCA1 | POLR2C | B3GALT4 | |
| NF2 | NLRC4 | ZNF764 | EPHB4 | PLEKHN1 | |
| ELL3 | GPR119 | TCTEX1D4 | DACH2 | RGS22 | |
| B3GAT3 | ZKSCAN1 | ELOF1 | FBXO16 | SEPHS1 | |
| RBM17 | EFHC1 | RAP2B | DENND1B | HPD | |
| PKM | NAT8L | LYNX1 | GNB5 | TP53I11 | |
| RCAN3 | CHMP4C | SLC7A2 | CLEC10A | ZC4H2 | |
| DEFB125 | MEMO1 | CLEC4E | SFR1 | LSP1P3 | |
| SENP5 | LINGO2 | NO_MATCH_62 | CAPG | PSG11 | |
| TUBB1 | SUN1 | GABRQ | RUNDC3B | XLOC_l2_010511 | |
| ARFGAP3 | FAF2 | FBXO32 | CFAP47 | NOA1 | |
| ATL2 | CPXM2 | CHST13 | ADIG | HHIPL1 | |
| NONO | LRRC41 | ANKRD26P1 | CHRNB2 | PLA2G2A | |
| SYNGR4 | ZFAND5 | RAB26 | KCNG1 | TMEM239 | |
| SF1 | SRC | B3GALNT1 | FBF1 | POPDC3 | |
| NUP62CL | CSPP1 | MTHFS | SSR3 | HIST1H3F | |
| KRT15 | DGKZ | OR13G1 | FAM81A | HYAL3 | |
| PINK1 | CEP83 | PON2 | C3orf58 | GNL3L | |
| HNRNPR | C17orf78 | DNAJC22 | S100B | GRK7 | |
| CCDC36 | PTPN3 | REEP5 | ZNF581 | TAP2 | |
| ANKRD23 | ZNF460 | CD74 | GYS2 | RHBDD2 | |
| THUMPD1 | CHSY3 | PIK3R4 | EEPD1 | COPS7B | |
| PGM3 | HARS | FAM161A | LSM3 | MREG | |
| WWP1 | NECTIN1 | GH2 | CATSPERE | DNAJC28 | |
| CHRND | ERVFRD-1 | PHACTR1 | ST3GAL1 | ST20-AS1 | |
| MPLKIP | KCNC1 | LANCL3 | CRISP3 | TIMM10 | |
| JAKMIP1 | TVP23B | SSX1 | SPPL2B | SEC13 | |
| Ddx17 | POGZ | ILF3 | SLC12A9 | SERPINB3 | |
| WDR93 | PIχ3 | PIWIL4 | SLC25A52 | ZC3H8 | |
| BTN3A1 | RAD51D | HOMER1 | SNRPN | GALNT17 | |
| SLC8B1 | C14orf80 | FUT9 | LRG1 | CBR4 | |
| MINK1 | SEC31B | TEX35 | ARNTL2 | SMCP | |
| DDX1 | VRK3 | 2-Mar | C15orf59 | ELMOD3 | |
| Pi4ka | SLC13A4 | C15orf54 | MC4R | TOMM20L | |
| SUPV3L1 | C1orf106 | CRABP1 | BVES | C20orf27 | |
| TIGAR | AKAP9 | LRRC55 | MINPP1 | SF3B3 | |
| PGR | FANCG | ZBTB39 | HSD17B8 | NO_MATCH_198 | |
| REC8 | ENDOV | OXGR1 | CTBP1-AS2 | RBM24 | |
| GSTM5 | CD226 | TOR1B | YKT6 | HBG1 | |
| ETV6 | MBL2 | SLC5A12 | PRSS1 | COX7B2 | |
| CNNM4 | IL18RAP | ASPH | RAC2 | KRTAP26-1 | |
| PPP1R3B | PYCR3 | ARL6IP6 | CSN3 | NO_MATCH_42 | |
| DCST1 | KRT6C | MEOX1 | TNK2 | PABPN1 | |
| 7-Mar | GABARAP | KCNIP1 | TLR8 | ZBTB44 | |
| ZNF624 | CHPF | B3GALT5 | KRT31 | TSGA10IP | |
| KLHL1 | GNE | TTC1 | LRP1 | FUT2 | |
| GID8 | GABRR1 | APRT | ATP6AP1 | PRUNE2 | |
| MLLT10 | RPA1 | SLC11A1 | NPY | WDR90 | |
| SPTY2D1 | RASSF5 | SLC35D3 | LACTB2 | CLN8 | |
| PPP2R2B | SNX15 | ARPC1A | INTS10 | CCL4L1 | |
| RMDN2 | CPOX | LIN7B | C1orf186 | PRKG1 | |
| ZNF426 | MAFF | HAO2 | GRB7 | HMGCS2 | |
| TMEM185A | NAP1L1 | ATP6V1B2 | KCNE4 | CSNK1A1 | |
| NO_MATCH_106 | CSMD2 | ZNF71 | FAM90A1 | OR51F2 | |
| QSOX1 | HEMGN | RPL34 | NRBF2 | OR4C12 | |
| MTDH | RRP1 | SERBP1 | ZNF184 | CHI3L2 | |
| SEC61A2 | SSH1 | EPHB6 | RIC8A | USP22 | |
| JAK1 | NTRK1 | OXSR1 | AK5 | XLOC_l2_000048 | |
| MTMR10 | LPXN | SKA2 | INHA | LOX | |
| GATAD2A | CD247 | HCFC1 | ACBD7 | RNF166 | |
| TMEM270 | YIPF7 | HIST1H4C | ALDH6A1 | TREML2 | |
| TGFBR2 | HOXC4 | LGALS9B | C3orf33 | ERLEC1 | |
| TMEM115 | Myo5c | CST2 | LIX1L | CES1 | |
| DND1 | TSTA3 | EME1 | GINS2 | FXYD7 | |
| DNAJB5 | AKR1D1 | DXO | GMPR | KLRK1 | |
| POU2F1 | SPCS2 | ACBD6 | ZNF772 | PI4K2B | |
| PCYT1A | BRCA1 | CD5L | TM2D1 | ZNF558 | |
| BCAP31 | SLC22A15 | PQLC2L | DGKA | MVP | |
| ARSF | RANGAP1 | MRPL10 | CLU | CINP | |
| CHMP6 | FAIM | PIWIL2 | RING1 | NEK4 | |
| CARNS1 | TLK2 | C19orf53 | ALDH3B2 | USP2 | |
| FSD2 | SDF2L1 | PLK4 | BZW1 | ADAT2 | |
| L2HGDH | ZG16B | SLC25A30 | RAD9A | XLOC_l2_006166 | |
| ADSL | PPM1F | ADAT3 | GSTM3 | DNM3 | |
| ZNF257 | ZNF423 | MIOX | ULK4 | LFNG | |
| GPS2 | COG3 | ACSL6 | HTR2B | PTCD2 | |
| PPP1CA | PIAS1 | DPYSL2 | SIM2 | DOK4 | |
| FGFR4 | MARVELD2 | COQ4 | SALL2 | NO_MATCH_94 | |
| C12orf4 | TMEM106B | B3GNT5 | C10orf111 | AADAC | |
| PRICKLE1 | RPL6 | RRAGD | FSBP | XLOC_013840 | |
| NOP58 | DNAJC3O | PSENEN | ETV7 | UBE2V2 | |
| TCEA2 | C17orf51 | MAPK7 | ERC2-IT1 | NRSN1 | |
| VMA21 | KMT2E | CCDC134 | ZNF133 | TMEM14B | |
| BCL7A | RALY | STBD1 | BEND6 | 1-Sep | |
| ZC2HC1A | DDRGK1 | PRCC | HMOX2 | LINC00242 | |
| MCOLN1 | LBHD1 | DYNLRB1 | SPRYD7 | SUOX | |
| ATG4C | RHOBTB1 | RBMX2 | TEX36 | ZNF439 | |
| PPP1R36 | CTBS | ENTPD7 | SBDS | MRPS11 | |
| CD63 | EYA4 | ATG5 | STX18 | ANGEL1 | |
| BBS4 | FAM46D | HIP1R | OR2AE1 | HDX | |
| BCL2L2 | NIPSNAP3A | HCAR3 | TMEM143 | TUBGCP4 | |
| CPSF6 | BPIFB2 | SULT1C4 | KRT20 | GLYATL1 | |
| SLC35D2 | GPR162 | MAPRE3 | NCSTN | DGKG | |
| PRSS23 | JAM2 | TBR1 | SAA1 | CD3G | |
| NO_MATCH_39 | CACNB2 | FAM30A | WASL | SDHC | |
| TCAF2 | KIF9 | UBQLNL | CFH | MOBP | |
| UNC93B1 | PCDH9 | ZNF404 | SNRPD2 | XLOC_l2_007488 | |
| C2orf49 | PTCH1 | DTX4 | ACOXL-AS1 | LOC554206 | |
| HAND1 | ADIPOR1 | CLSTN2 | GEMIN6 | CRTAP | |
| SLAMF7 | SRFBP1 | GPR21 | TFB2M | CYTH4 | |
| NO_MATCH_46 | AKR7A2 | DEFB127 | LOC107984086 | TOMM40 | |
| MID2 | TCP11L1 | GPR149 | RTN1 | NECAB1 | |
| OR10K1 | E2F3 | THOC3 | KRT38 | ATG4D | |
| UBE2J1 | CD200R1 | CSN2 | PAQR7 | EIF3F | |
| F10 | DDX59 | NDUFS3 | MTPN | PPP2R1A | |
| CRISP2 | CPNE2 | CLIC2 | TADA2B | NO_MATCH_80 | |
| MFSD2A | ZDHHC22 | SLC25A22 | PSTK | ZNF331 | |
| C1orf112 | PIGT | EIF3H | NMUR2 | RBKS | |
| HCFC2 | EXD1 | QRSL1 | PIP | GXYLT2 | |
| LPP | NET1 | LIMSI | KCTD9 | BTF3 | |
| MXD3 | CNPY4 | IFIH1 | BEST1 | NO_MATCH_96 | |
| PRDM1 | C9orf153 | FAM151B | ISOC1 | PLCD3 | |
| TMEM101 | GCNT1 | GPR135 | CSF1R | COX20 | |
| BEND3 | GPR6 | NKPD1 | NR1H4 | PRIMPOL | |
| TP53TG5 | PRUNE1 | DAOA | CYP3A5 | MYL6B | |
| IFNL3 | CAPN13 | STAP2 | LNP1 | STAC | |
| TMEM35B | KIFC3 | NFYA | OR2K2 | LOC107987205 | |
| SCG3 | TSSK1B | TNNT1 | JMJD1C-AS1 | AHNAK | |
| DDX54 | ZNF2 | WDFY3 | MRAP | HMGCR | |
| HAVCR1 | LHX9 | DLG3 | MTHFD2L | KMT5A | |
| IKZF2 | ZNF449 | ENO3 | CD300A | RNF139 | |
| ZMYND10 | TMEM174 | ITIH3 | PSMB4 | NXNL2 | |
| KCNJ5 | CES3 | TMEM208 | TMEM35A | TYW3 | |
| PCSK5 | RLN1 | KRT73 | TMX2 | SIRT2 | |
| TFB1M | ZNF821 | HES6 | PXDNL | LYZL1 | |
| CDH7 | TMEM44 | EGLN1 | ZDHHC20 | ABAT | |
| CLGN | ACTR1A | TUBB2A | OR2L2 | TMEM27 | |
| OR10S1 | MIIP | CXorf57 | HIGD2B | RPL35 | |
| GNA15 | GPR88 | ZBTB47 | PRSS57 | ZC3H12D | |
| Zfp317 | CYP4V2 | TIGD5 | VDAC1 | CACUL1 | |
| SERINC1 | TNIP3 | XLOC012148 | YWHAE | DCP1A | |
| PPP1R16B | SHISA5 | SQOR | C15orf39 | PBRM1 | |
| NO_MATCH_265 | SLC22A5 | RYBP | LIPG | CROT | |
| ATG16L1 | PLEKHG3 | NO_MATCH_259 | SUCNR1 | TSPAN1 | |
| FKTN | CLDN8 | PREPL | OR10W1 | NCOA4 | |
| ELOVL6 | TMEM186 | HEMK1 | TTK | OAS2 | |
| XRN2 | RWDD2A | NDRG2 | RNF182 | MRAP2 | |
| STX1A | TNFSF18 | ZNF329 | LOC442132 | PNKD | |
| LYSMD4 | FMN1 | ZNF846 | CEP72 | MICU1 | |
| PARD6B | TRPT1 | SPATC1L | STXBP6 | C2orf68 | |
| VEZT | HIP1 | DHRS4 | TGFBI | NDUFB10 | |
| ETV4 | PPP4R1L | METAP1D | TTC6 | PTRHD1 | |
| CAV3 | BSPRY | AKNAD1 | CLEC3A | NR1H3 | |
| TRAF3 | KIAA1257 | MMP8 | COMMD8 | CTAGE1 | |
| SMC1B | CDCA7 | MMP3 | GRPEL1 | TMIE | |
| SHISA3 | OTOR | NME7 | PSMC4 | STAM2 | |
| TRAK1 | AIMP1 | C22orf46 | SLC44A5 | TESMIN | |
| Zfp777 | ATAD2 | LRGUK | OR5K2 | CXCL6 | |
| STK31 | TOM1L1 | GUSB | CCDC7 | KLHDC10 | |
| USH1G | VCX3A | TMEM33 | CCT8L2 | RNASE1 | |
| TUBG2 | DOC2A | TYRO3 | ASPSCR1 | TES | |
| DEDD | MAGEF1 | PMM2 | URB1-AS1 | FPGT-TNNI3K | |
| S100PBP | Bnip3 | IL12RB2 | XLOC_l2_001669 | H2AFX | |
| AKR1B1 | DENND6A | MAP2K3 | YPEL1 | SOD1 | |
| FAM135A | C7orf31 | EXOC8 | PSG1 | SECISBP2 | |
| UCP3 | RPIA | HMGB3 | C11orf1 | TADA1 | |
| FAM217B | FDFT1 | DAZ3 | COASY | POLR1C | |
| NUDT10 | YWHAQ | EXO1 | KLHL11 | TTLL9 | |
| FBXO17 | DIRC2 | ANXA8L1 | AKR1C3 | ODAM | |
| DSE | XLOC_l2_007271 | ANXA10 | FAM171A2 | TRIM62 | |
| NAA15 | ZFYVE21 | HIBCH | TNNT3 | RASGEF1C | |
| CACHD1 | SEC14L1 | DHRS4L2 | PSMB1 | CENPA | |
| TMEM209 | C16orf72 | CDR2 | Atr | C16orf59 | |
| CHN2 | AGRP | SRP68 | MANF | MBLAC1 | |
| SLC5A7 | SLC20A1 | MCOLN2 | BCOR | DDX60 | |
| OSR2 | ZFP28 | VPS50 | CPNE5 | ZNF397 | |
| LRRCC1 | RPS7 | SCAMP2 | PDIK1L | TINAGL1 | |
| ZNF177 | RNF43 | MZB1 | NPL | LYSMD2 | |
| ANGPTL3 | LOC440461 | LILRB1 | GOLGA4 | NO_MATCH_145 | |
| C12orf65 | DMTF1 | TRIML2 | LOC107986810 | ERGIC2 | |
| PRMT1 | SERPINI2 | SIAE | ARL16 | SMOC1 | |
| GNAI3 | CALHM3 | ALDH1L1 | PSG2 | EEF1AKMT1 | |
| SUCLA2 | TMEM200A | LGI2 | CHST4 | TWIST2 | |
| TSPEAR | HEXB | SERPINB11 | ENY2 | CXADR | |
| RNF128 | KCNN4 | FAM84B | LYPD5 | FABP3 | |
| MRGBP | CLSPN | XLOC_l2_011954 | IL18 | PRKAG3 | |
| FAM83A | FERMT2 | PTPDC1 | REEP2 | TRIM10 | |
| CPA4 | GNRH1 | GIMAP7 | EMD | Thap12 | |
| PHOSPHO2 | OXTR | ANXA1 | FKBP1A | MTG2 | |
| STRN | PXN | BCL2L14 | TMEM106A | PSG8 | |
| APOBEC3F | CCDC26 | ARHGAP6 | FAM117B | NUDT11 | |
| MB21D1 | SDHA | BMP2K | STK32C | GIF | |
| TRIM4 | MFGE8 | TSEN2 | ERGIC1 | NUP88 | |
| ATXN1 | YAE1D1 | MEDAG | MESP1 | DIO1 | |
| ADAMTS12 | CLASP1 | RANBP3 | TRAK2 | LGALS8-AS1 | |
| SATB1 | TMSB10 | DKK1 | SLC25A11 | TAF1D | |
| TMIGD3 | WDR33 | LDLRAD4 | RIT1 | AMOTL2 | |
| RPAP3 | TGIF1 | C6orf106 | DCPS | RHOXF2 | |
| PNMA5 | ANK1 | POLE2 | ADAM7 | NHLRC2 | |
| TBX18 | RARG | ZHX1-C8orf76 | ACP1 | NUP58 | |
| SPG7 | TAF9 | ZDHHC16 | RND2 | UBA6 | |
| RIPK2 | CD14 | LOC284009 | C8orf33 | SLC25A15 | |
| SLC9A3R2 | RWDD1 | XLOC_001973 | LCN12 | HSD17B2 | |
| GPT2 | LINC00636 | CHMP3 | ELAC2 | MELK | |
| ANXA2R | ANKLE2 | PRLHR | HMG20B | NDNF | |
| CYP4A11 | IQCB1 | MERTK | MB | NUTF2 | |
| ALG5 | TSGA13 | XPNPEP3 | AKT3 | NUBP1 | |
| ZNF33A | SLPI | C2orf50 | MGLL | CFAP100 | |
| SLC7A3 | MAP2K5 | ASB16-AS1 | PRPF19 | ANKS3 | |
| TTYH2 | PRR23B | SMIM5 | UBQLN4 | C21orf59- | |
| | | | | TCP10L | |
| SFT2D1 | MZF1 | NOX5 | ATRIP | KDSR | |
| TMEM156 | RPL37A | CHST11 | TUBA1C | Zyg11b | |
| MSX1 | HCST | RHBDD1 | GLT8D2 | DCN | |
| BIRC2 | LAIR2 | DNAL1 | TMEM159 | KCNAB3 | |
| TRUB2 | KAT7 | RPS4X | MAGEE2 | PTPRR | |
| Kat14 | CCDC140 | LOC107987559 | ACAD10 | FERMT3 | |
| PM20D2 | KIR2DS4 | ATAD3B | BMP7 | ZBTB6 | |
| EPS8L1 | CASP1 | CAPS2 | ATP9B | C1QC | |
| PPM1G | PAX3 | DYRK2 | HOGA1 | LRRC8D | |
| TULP1 | OPN4 | NECAP1 | IL20RA | Srgap3 | |
| ALG11 | FGF7 | NELLI | SLC25A51 | SEMA4D | |
| GPR12 | CDK5RAP1 | NOL9 | CCDC120 | DPH7 | |
| DUSP16 | COPS6 | QPCT | KRBOX4 | ACAT2 | |
| NO_MATCH_203 | CTBP2 | NDUFAF4 | C21orf58 | LINC01588 | |
| PARP12 | BCAP29 | PRKG2 | HEATR1 | HIF1A | |
| CHGA | LOC107984058 | DBN1 | OR6F1 | TADA2A | |
| FGF14 | FAM118A | RASSF7 | PHKG2 | ITPRIP | |
| PCDHA12 | ZNF80 | TMEM70 | ZNF302 | GDAP1L1 | |
| STX8 | NPR2 | ZNF267 | RPL17 | GPRC5B | |
| MME | CYP11A1 | GPR26 | SLC22A12 | SYK | |
| LALBA | RBBP8 | RTP5 | CHST6 | GPR157 | |
| STX2 | FBXW11 | MVD | VSTM1 | SPINK7 | |
| IPPK | SPIN1 | PON1 | TMEM47 | NO_MATCH_86 | |
| STYX | NUP50 | UEVLD | TXLNA | DEFA3 | |
| OR10H1 | RDH11 | HSPA1L | WNT9A | C12orf40 | |
| MAGEB3 | PRKACB | TIMM23 | PALM | PPP1R2 | |
| RAB14 | ZNF747 | KCTD17 | TFF3 | ECT2 | |
| FMO4 | SMARCC2 | LOC102724151 | H2AFV | NACA2 | |
| PTPN14 | MAGEB4 | DGAT2 | PDK1 | STS | |
| LIAS | TOX4 | ATP6AP2 | TMEM41B | CNRIP1 | |
| FGG | DPP6 | CD164L2 | OR1S2 | C11orf42 | |
| AP1AR | MIPEP | MRPS30 | ZNF112 | LONRF1 | |
| POMT2 | KRTAP12-2 | LRPAP1 | RSL1D1 | PKDCC | |
| NO_MATCH_227 | SCO2 | UQCRC2 | TM6SF1 | KLHL31 | |
| SLC18A3 | UBE2R2 | NSMF | SWAP70 | HUS1 | |
| GPR160 | SNRPG | ABHD5 | KIAA2013 | RABL2A | |
| SLC30A1 | RSPH1 | NECTIN4 | SLC23A1 | EPHA6 | |
| SPDYE4 | MAK16 | GRID1 | BST2 | MGC16025 | |
| NCEH1 | UNC5CL | DNALI1 | FTMT | NO_MATCH_261 | |
| FAM118B | CCNE2 | NDUFAF5 | EXOSC110 | SGO1 | |
| SEC22A | ASIC4 | METAP1 | FCRLA | SCN1B | |
| MEX3B | SPINK1 | SCOC | NR1H2 | SPHK2 | |
| TBC1D2 | KRT40 | TMEM5 | PACSIN3 | TMPRSS12 | |
| GRK2 | NT5C3B | C3orf36 | ACTR6 | ODF1 | |
| HPRT1 | STPG2 | GJA5 | MAPRE2 | ADIRF | |
| GTF2H3 | ZNF454 | TEKT3 | LIMS2 | TRABD | |
| PDCD4 | TMED10 | DAXX | L3MBTL3 | FGFBP2 | |
| ENAH | TIPIN | LOC105376844 | CAMKK1 | LGALS7B | |
| MAGEC2 | THRB | PPARG | FBXO4 | C11orf71 | |
| SPDYE1 | LARP4 | GALNT5 | ABHD17A | RPL30 | |
| TRAFD1 | FOXG1 | PAK1 | CLECL1 | PIGG | |
| ITGA9 | SNRPD1 | CXorf66 | TDP1 | ARHGAP28 | |
| TIMP4 | GTF2A2 | ZNF276 | TNFSF9 | Usp48 | |
| GCHFR | PRKRIP1 | CCDC17 | CCDC65 | C19orf57 | |
| RBPMS | IDI2 | NDST3 | PRPS1 | APCDD1 | |
| TMEM263 | ALKBH1 | NO_MATCH_45 | NOV | PHC3 | |
| KHNYN | MORC2-AS1 | SMCHD1 | REPIN1 | KCNQ2 | |
| FGGY | HSD11B1 | CNNM1 | SOCS4 | CPED1 | |
| KRTAP8-1 | RD3 | SDC3 | FAM189A2 | NPHP1 | |
| CLDN14 | CDKN2AIP | EVA1A | TAS2R31 | GATAD2B | |
| WDR36 | CBR1 | LGMN | TUBB4A | ALG3 | |
| RARRES2 | NSUN7 | CLIP1 | ISCU | PTPA | |
| GRINA | RALYL | ZNF32 | GOLGA7 | ANKS1B | |
| RNF20 | MAP3K9 | PENK | BTN2A2 | HINT2 | |
| NO_MATCH_274 | NCF1 | GALNT12 | KLC3 | ARRB2 | |
| TRMO | CCDC141 | MSS51 | OR14I1 | C8orf46 | |
| KIFAP3 | WSB2 | JPT2 | DCAKD | IGFL2 | |
| MTMR7 | APCDD1L | TAGAP | APBB2 | LATS1 | |
| NANOG | NO_MATCH_222 | GNPDA1 | NO_MATCH_108 | C2orf82 | |
| USF1 | SLC35E2B | UFC1 | XLOC_011808 | NO_MATCH_83 | |
| CDC25A | HIST1H2AA | VSIG8 | APOC3 | RNF217 | |
| ZNF18 | ACP7 | IGFBP5 | SRGN | INSR | |
| CDH6 | TAS2R43 | TDP2 | LIPA | SMPD3 | |
| MRPS31 | GTF2F1 | WDTC1 | LIMD2 | SUSD3 | |
| PDCD6IP | ZNF562 | ERN1 | LINC00895 | CST9L | |
| PAPD7 | FGB | PYCARD | UFD1 | PAX4 | |
| PPP1CB | EPHA8 | UBXN2B | COCH | DHFR | |
| NXF1 | CYP2W1 | TUBA3E | POLR2F | KCTD21 | |
| COX7A2L | MED21 | CD248 | HYAL1 | PTPN22 | |
| MMP14 | QKI | PLEKHA1 | RAB34 | RDH5 | |
| GPR3 | SLC17A3 | PLEKHS1 | OR2L13 | MTRF1L | |
| PHF20 | ARRB1 | PTPRO | ZNF287 | MT2A | |
| SMG8 | ATP5F1 | HLA-G | Naa10 | ANKRD13D | |
| ALAS2 | RARRES3 | GBP5 | USP6 | LCE3D | |
| POLH | ZKSCAN8 | FN3KRP | PACRG | ARAF | |
| CDR1 | NFU1 | SUSD4 | MAGED2 | ZNF641 | |
| SNX18 | CDC42EP1 | SPCS3 | PSMC1 | PIAS4 | |
| HMX2 | TLCD1 | KMO | MED22 | ARHGAP5-AS1 | |
| SCGN | KCNA10 | NIT1 | S100A11 | FZD10 | |
| OVOL1 | KRTAP13-2 | HSPA6 | SFXN4 | ASXL2 | |
| GDA | TEX2 | OTULIN | KCTD16 | CAPN5 | |
| SVOP | NKAIN1 | COL23A1 | CYGB | Sgsm1 | |
| ZMYM4 | MPP2 | EPS8L2 | HSPB6 | NO_MATCH_228 | |
| ANKRD46 | TBC1D19 | NR2C2 | FASTKD2 | ZC3HC1 | |
| RCBTB1 | LARP4B | SSX2IP | P2RX4 | ZFHX2 | |
| CCDC6 | XLOC_l2_000915 | PIMREG | FAM3B | XLOC_l2_009492 | |
| DOCK4 | Aak1 | PPIE | CSAD | DMRTA1 | |
| CSTL1 | CIB2 | MAP3K7 | CNMD | LHX8 | |
| NR0B1 | FCF1 | CEP104 | TBC1D3J | SERPINB13 | |
| WWP2 | MDM4 | A1BG | NBPF6 | MTUS2 | |
| CCDC122 | TMEM184B | PRKAG2 | KRT81 | PIGZ | |
| ESM1 | MCPH1 | METTL2A | JTB | NO_MATCH_143 | |
| PRKAR1B | DONSON | LINC01561 | ZNF563 | TUBE1 | |
| PIF1 | RGL4 | VPS4B | RNF185 | CPNE8 | |
| KCTD8 | FAN1 | HIST1H2AL | ULK2 | FRG1 | |
| DNASE1L2 | TPR | NO_MATCH_126 | C9orf64 | IGIP | |
| GCDH | SBSPON | SDHB | SMIM7 | Akt1s1 | |
| SNAI3 | DPH2 | SNPH | TRAM1L1 | ATXN7L1 | |
| GDF11 | SLC7A9 | OR12D3 | NO_MATCH_119 | TRIM11 | |
| HORMAD2 | RNF26 | CYP2J2 | STH | FHL3 | |
| ACAA2 | IFNA7 | AIFM2 | NO_MATCH_75 | FEN1 | |
| SEMA4A | PRKAB2 | TCTE3 | MISP | ENO2 | |
| ZNF548 | CSNK2A1 | LSM1 | LPCAT4 | CDC20 | |
| ACER1 | FAM163A | LTF | E4F1 | SMPDL3B | |
| TMEM259 | CLCNKB | USP16 | SNRPC | SRP14 | |
| GNS | FLRT1 | MAS1L | KCNS2 | WFDC10A | |
| PLEKHB2 | PNPO | ZFC3H1 | UQCC2 | TFPT | |
| CHEK1 | PLB1 | BUD13 | PCDHB4 | SYTL1 | |
| NAF1 | TRMT61A | NDUFA10 | SESN3 | PLPPR1 | |
| RLIM | HYPM | TPGS2 | NO_MATCH_55 | UBXN1 | |
| TSSK3 | SULT1E1 | POM121 | APOA5 | SCARB1 | |
| BHMT2 | UBAP1 | SLC35A4 | SH3BGRL2 | B3GNT3 | |
| RBMY1J | PODNL1 | TNK1 | GADD45B | FARP2 | |
| EXOSC4 | ATP5H | G6PC | PCOTH | CRYGA | |
| FRMD6 | ZNF597 | INHBC | FAM13C | ZNRF4 | |
| TBC1D22B | RUNDC3A | CDC42SE2 | LOC102724652 | CCDC155 | |
| IKBKE | SFTPC | FBXW4 | DNAH17 | IFT43 | |
| LOC153684 | NLK | MSANTD2 | TBP | RGS3 | |
| OXNAD1 | NFIB | RSRC1 | RCAN1 | PCBP3 | |
| LHX6 | GRIK2 | ANTXR1 | TMEM266 | FBXO10 | |
| LSM14A | RPA2 | SLC2A12 | CASQ1 | CENPN | |
| NDUFAF7 | POLDIP3 | CCDC88C | SARS | CORO6 | |
| SORCS3 | Rictor | AQP12B | RTL8C | HECTD2 | |
| PRRG1 | C9orf106 | TYW1B | PRELID1 | UQCRB | |
| FZD9 | NFIA | GSTZ1 | DYM | SCEL | |
| TEPP | RARS2 | NIFK | CPA6 | RPL22L1 | |
| RGP1 | MED18 | CNTN5 | OR2A25 | CA8 | |
| CNOT9 | FPGT | TM2D3 | FTO | ZNF677 | |
| NO_MATCH_3 | RIDA | SMAD7 | PF4V1 | ETS2 | |
| AIFM1 | EIF1B | ZNF595 | ATAD3A | TFPI2 | |
| HRASLS | PLCB2 | PIGY | XLOC_005142 | INIP | |
| TKFC | OR4N4 | NARF | HPSE | FADS2 | |
| HDAC5 | DLC1 | BLCAP | LYZL6 | SFTPA2 | |
| Fam122a | ATCAY | SF3B2 | UTP15 | TM2D2 | |
| NASP | KRT2 | ATRAID | ITM2A | MTHFD2 | |
| ABLIM1 | XLOC_l2_006014 | SLC16A10 | PDLIM5 | TPRX1 | |
| RCC1 | PLRG1 | SRI | DNAJB9 | LOC440700 | |
| LUZP2 | UIMC1 | ESR2 | KIAA0825 | CATIP | |
| LDHC | KRTAP4-1 | PAAF1 | C22orf39 | HFM1 | |
| FOXN2 | GAPDH | KANK4 | ZNF550 | ZBTB25 | |
| HAUS4 | MED10 | ENO1 | GDF5 | NUDT5 | |
| ZNF101 | OR5M11 | Olfr981 | RPL28 | OR10H2 | |
| LOC102724159 | FMC1 | DNAJB11 | IQCA1 | RASD1 | |
| NO_MATCH_205 | BTK | C10orf91 | SNUPN | HGF | |
| TWNK | ALX3 | TESPA1 | C1orf35 | CDKN2AIPNL | |
| NIF3L1 | ABHD8 | CABYR | CRYZL1 | TNF | |
| PRPF38A | LCN10 | PRSS38 | DUS3L | SPTSSA | |
| HIRIP3 | CLUAP1 | HOXB1 | WDR86 | OTOP1 | |
| MFSD7 | CASC4 | PCGF1 | CCDC78 | OSBP | |
| BOLL | PHF6 | PARTI | RFTN2 | PTPRN | |
| FZD6 | COQ3 | DEPDC1 | TAS2R9 | STOM | |
| MAP3K8 | ALKBH4 | FAM109A | ARHGAP17 | SPTLC1 | |
| ACRV1 | POLE4 | PRPF3 | NO_MATCH_178 | CCDC183 | |
| SLC36A4 | TTC23 | RPL7 | COPS4 | DUSP4 | |
| CTCF | PAN3 | CDH8 | UTP23 | MED9 | |
| P3H2 | ACSL5 | C16orf52 | GPR148 | GK2 | |
| TM0D2 | PDZRN4 | TPCN2 | ZC3H12B | CCDC148 | |
| TTLL4 | STRN3 | GPR52 | RBP7 | DHPS | |
| DNAJC17 | LINC00526 | SPP1 | C18orf54 | ADCY2 | |

TABLE 4
ORF Negative Screen Hits (Gene Symbol)

| Gene Symbol | Gene Symbol | Gene Symbol | Gene Symbol | Gene Symbol |
|---|---|---|---|---|
| IDI1 | TKT | AGTR1 | SPACA1 | C1orf27 |
| MTHFR | RABL3 | TAC1 | TMEM59 | APH1A |
| SLC24A5 | ATG9B | ARG1 | STOML2 | HLA-DOB |
| ATP4B | OR51Q1 | ZNF575 | RNF144A | AP2B1 |
| SLC8A3 | GBP6 | NO_MATCH_107 | NEGR1 | TXNRD1 |
| ACVR1C | PRSS48 | GSK3A | NO_MATCH_190 | C17orf53 |
| FANCD2OS | RHBG | GJA1 | KRTAP10-7 | TAS2R20 |
| OR10X1 | C2orf48 | GALNS | EXOSC9 | FZD4 |
| CDS1 | EXOSC8 | PIK3CD | REG3A | TMC4 |
| CCNJ | GAR1 | RALGAPA2 | ZNF322 | JAK2 |
| TUSC2 | MS4A2 | METTL6 | NKD2 | SIAH1 |
| CIB3 | SERF2 | IGFBP1 | HERPUD1 | LAMP1 |
| CCNG2 | NUDT1 | CYP1A2 | MMP7 | CHD9 |
| AURKA | YIF1B | CDC25B | C16orf92 | IP6K1 |
| TMED3 | TXNDC11 | Rasl10a | S100A7 | AFMID |
| XLOC_005466 | SERPINE2 | TMEM230 | LURAP1L | ANKRD42 |
| BDKRB1 | AQP7P5 | HSFY2 | CNFN | CD70 |
| TEFM | DUSP9 | PANX1 | SLC22A23 | TMEM220 |
| RPS19 | TANK | C11orf74 | KCNN2 | PUSL1 |
| AMN1 | SMAD9 | NPHP4 | ANKS6 | RIOK2 |
| SYPL2 | KLK6 | RNASET2 | ZFPL1 | FAM196A |
| EID3 | MS4A13 | OSTN | LINC01580 | NUDT9 |
| BLOC1S5 | TMEM242 | LSM7 | ECEL1 | CD28 |
| AGT | PI4K2A | VWC2L | SLC2A3 | SP100 |
| ANAPC7 | MPP6 | TRIM43 | RDM1 | MXI1 |
| NO_MATCH_20 | C19orf48 | OR6C4 | CD36 | FRMD5 |
| PDLIM3 | BPNT1 | DERL1 | RCN2 | GPATCH2 |
| EEF2 | NUDT18 | LRRN2 | NO_MATCH_28 | NO_MATCH_175 |
| DNAJC8 | DRD2 | UBE2J2 | HDDC2 | SYT2 |
| HEXIM2 | ADRB2 | DCAF4L1 | SIGLEC6 | HARS2 |
| ITGB3BP | KCNMB4 | GHRH | C8A | ARMCX1 |
| SUMF2 | COMTD1 | ABCG2 | ATXN7L3 | GCFC2 |
| CHCHD10 | UGT2B4 | GZMM | WNT16 | SUDS3 |
| KIF16B | KIAA0895 | LINC00486 | CSNK1G3 | TMEM9 |
| RTN4IP1 | OTUB1 | CARNMT1 | RTKN | NAT14 |
| OR10G8 | M6PR | TMEM241 | ATP6V1G1 | FAS |
| CYR61 | NDUFB3 | PADI1 | CD79B | ZNF354A |
| TTC25 | NFE2 | XLOC_l2_009136 | PDGFB | FOXRED1 |
| NPM1 | NO_MATCH_260 | KRCC1 | DENND2A | EQTN |
| NOD1 | ZNF181 | GAGE2E | KCNMB1 | MARK2 |
| KITLG | TFG | KL | XLOC_013923 | ISYNA1 |
| ANAPC15 | KCNMB2 | PNPLA6 | MRTO4 | NO_MATCH_148 |
| HBE1 | LAMC1 | PAPLN | XLOC_l2_010863 | CEP76 |
| CCDC25 | ACTR10 | SLC9A9 | IDH3A | STAT3 |
| MAP3K20 | EAF1 | SPCS1 | BABAM2 | PLD6 |
| SIPA1L2 | MOB3B | KRTAP3-1 | RABEPK | SNIP1 |
| CHKB | HYOU1 | SUPT20H | PRB4 | IMPDH2 |
| ARHGEF35 | FAM149B1 | TMEM216 | RUSC1-AS1 | NO_MATCH_158 |
| CHN1 | ZNF285 | NO_MATCH_44 | RCAN2 | TACR3 |
| TROVE2 | MRPL20 | DRD5 | TMEM196 | SLC31A1 |
| OR5P2 | GRAMD1B | FMR1 | LATS2 | WWOX |
| SDS | C6orf48 | TRPC1 | C9orf16 | MSL2 |
| RNF38 | TMEM116 | KCNK17 | TRIR | GTPBP10 |
| CASP6 | ASL | CFAP20 | NO_MATCH_114 | C19orf70 |
| THAP1 | LCLAT1 | TRPC7 | DSCR4 | AMY2B |
| LOC151760 | PAICS | CDC26 | CYP7B1 | MAOB |
| EPM2A | RPF1 | CERS3 | EPN3 | LAG3 |
| LINC00305 | RNF126 | RXRG | ZFYVE19 | ZNF658 |
| EPDR1 | GJD2 | NGDN | GRIP1 | ADRA2A |
| STX19 | LINC02449 | SLC6A5 | SKA3 | CPNE4 |
| BCL2L12 | MSI2 | VHLL | ZNF212 | ETFDH |
| RAB33A | WISP2 | POP5 | LRRC61 | RBM26 |
| TMC7 | MRPS33 | KRTAP9-3 | TSLP | MAMDC2 |
| OR2B6 | MLF1 | MRPL11 | ZSCAN26 | RELT |
| SFTA2 | RAB17 | WDR77 | NMBR | PSMA8 |
| SLAMF6 | DMAC1 | R3HDM2 | MVB12A | GMCL1 |
| NYAP2 | NPM3 | STK32A | BTC | NO_MATCH_95 |
| OSMR | TRAPPC6B | ACOT8 | PYGB | ETV1 |
| DHDH | XAGE3 | RPP40 | ASCC1 | PTPN18 |
| Dmc1 | ODF2L | EPHA4 | SLC2A14 | LARP6 |
| ZNF148 | CYC1 | APOPT1 | SND1-IT1 | CCNL2 |
| TMEM206 | SLC25A48 | B2M | VDAC3 | ST3GAL3 |
| CTH | TGM4 | TLNRD1 | CASTOR1 | C11orf16 |
| AIF1L | DCANP1 | SLC38A6 | CHUK | NPB |
| KLK7 | FTHL17 | NO_MATCH_13 | UTS2 | SLC26A5 |
| RASAL3 | SH3GLB2 | OR51G2 | NAIF1 | CPSF7 |
| PIGX | NFYC | OR2T27 | NO_MATCH_157 | DKFZP434K028 |
| COA5 | HPX | SDHAF1 | RPS2 | NAT9 |
| FAM166A | TMEM100 | SPATA8 | DHX37 | INSIG2 |
| STKLD1 | FCER1G | FBXW12 | IMP4 | PRKCQ |
| TAX1BP3 | MEAF6 | GAL3ST1 | CLCN4 | ST7L |
| ILDR1 | TWISTNB | Trim41 | TRIM13 | BCKDK |
| MLIP | HYI | TP53INP1 | SPACA9 | EIF6 |
| UPK3B | PHF7 | SNX17 | TMEM53 | NOLC1 |
| FABP7 | BIVM | RRAGC | CRK | GPAM |
| CYP4F12 | USMG5 | KNSTRN | HTR1B | OSGEPL1 |
| OR2F1 | SDF4 | BAMBI | PNP | RPUSD2 |
| PLXDC2 | VSX2 | NME4 | GCLC | RNASE7 |
| DEGS1 | SERF1A | KCTD3 | RPL13A | ANP32A-IT1 |
| CHIC2 | ZNF468 | LOC107984056 | CEP290 | COX4I2 |
| TMX1 | POLD2 | NO_MATCH_164 | ISG20 | RDX |
| CHCHD1 | THEM6 | RNF32 | FAM153B | KIR2DL2 |
| EPHA7 | EPO | Clpb | LINC00479 | SLC25A25 |
| PIP4K2B | MLC1 | PPIAL4C | SLC25A39 | NHLRC4 |
| TBCCD1 | CLRN2 | GTPBP3 | SNHG11 | PMCH |
| DCUN1D1 | RPP14 | BAIAP2 | CPXCR1 | CBX7 |
| PRRG4 | CTSS | NUMA1 | PIK3CG | LYAR |
| TNKS2 | PGPEP1 | USP28 | CCR7 | CTSV |
| PVALB | PGM2 | IDNK | PDLIM4 | TSC1 |
| SAE1 | PDHX | BCO1 | UBE2V1 | JPT1 |
| NAXE | SPG11 | GEMIN7 | MTFR1 | QARS |
| ATP11B | CENPL | HSD17B7 | ZMAT5 | DDX49 |
| SORD | PNPLA2 | UQCC1 | MAN1C1 | GPR27 |
| MSRB3 | KCTD21-AS1 | TUBGCP2 | SKA1 | C6orf141 |
| ATF6B | ERG | RPLPO | LRRC20 | TIMP3 |
| CHGB | NO_MATCH_79 | COX17 | AGXT | DIS3 |
| SNRPB | SGMS2 | ATAD1 | HIST1H2BJ | TRMT11 |
| MECR | MTERF1 | MELTF | IP6K2 | PKN3 |
| MMD2 | NPPA | PIGK | FAM71F1 | CMSS1 |
| CATSPER2 | RSPO3 | CRYBA4 | CRMP1 | HMGCL |
| SLCO4A1-AS1 | MAFG | SYAP1 | TRMT112 | DAND5 |
| GYPB | SLBP | ACOT6 | RPL37 | ICK |
| RWDD4 | ATP5S | SOCS2 | ALG14 | SRSF12 |
| ELMOD1 | RPS10 | SCYL3 | P2RX7 | RNF114 |
| PSMA5 | CATSPER2P1 | KCTD5 | CAPNS1 | MKS1 |
| NCF2 | CENPO | PSMA2 | ENPP5 | SYNE4 |
| GPX7 | IFNLR1 | CDKN2A | PLIN5 | ANAPC5 |
| SLC30A8 | OR2G3 | NO_MATCH_242 | XLOC_006950 | NEFM |
| ZNHIT2 | MOCS1 | TMUB1 | BBS9 | GPR132 |
| TYR | SMPD1 | LGALS3 | ZBTB24 | ZNF324B |
| ANKRD34B | TK2 | HSPA4 | LOC105371303 | ALDH1B1 |
| EBP | GDI1 | TMEM267 | LZIC | TMEM248 |
| SH3BGRL3 | TRPM1 | CAMK2G | GSAP | TMEM81 |
| ERAL1 | SELENOV | CCDC167 | NO_MATCH_125 | COLEC12 |
| IL32 | IFIT2 | SPATS1 | DNAJA1 | CNR2 |
| MOAP1 | C19orf12 | VWC2 | NO_MATCH_234 | NO_MATCH_30 |
| WLS | C12orf66 | UBE2W | CCDC96 | CXorf38 |
| ADAT1 | NO_MATCH_276 | BMPR2 | CYP2C18 | NO_MATCH_156 |
| BIN3-IT1 | RTL3 | ZNF542P | TEAD2 | ORAOV1 |
| ACADL | TUBB2B | TRAF4 | SGTB | CRTC1 |
| ATF2 | METTL1 | NOTCH2 | KLK2 | CYP1B1-AS1 |
| COQ5 | PARP15 | PSTPIP1 | KCNJ3 | XLOC_l2_009493 |
| HPGD | CTSG | C7orf34 | HASPIN | AGTR2 |
| NT5DC3 | TEAD4 | S100A10 | AMBN | NDUFS1 |
| BMF | OR5AK2 | MT4 | SELENOF | HLA-DRA |
| PLCXD2 | TMEM165 | ZNF398 | SMAP1 | DSC2 |
| PFKFB1 | C14orf177 | INS-IGF2 | FSCN2 | IQUB |
| CDK5R1 | KCTD6 | ALDH1A3 | KRT19 | CCKAR |
| LRCH1 | TTC33 | ZSCAN16 | RAPGEF4 | SUFU |
| PLEKHJ1 | RAB3IL1 | FADD | ZNF254 | TOMM70 |
| SULT6B1 | SPESP1 | COX16 | MSMO1 | PCGF5 |
| SEC61B | KIF12 | TCF23 | BANF2 | MRPL51 |
| RCN3 | FTCD | ATMIN | DCUN1D4 | CCDC150 |
| ADIPOQ | NO_MATCH_64 | RADIL | REGIA | RBM43 |
| CDC45 | VAT1L | H2AFY2 | ITFG2 | PELI3 |
| UTP11 | DISC1 | TAS2R38 | TNFSF13 | EFNA4 |
| PITPNB | NO_MATCH_250 | NO_MATCH_105 | C12orf74 | ZNF582 |
| AGPAT1 | SMG6 | C18orf21 | WDR19 | CCDC178 |
| LINC00574 | KIAA0408 | TAAR5 | RAG2 | PPAT |
| XLOC_l2_009889 | ABLIM3 | LGALS8 | FTL | MBTPS1 |
| RXFP4 | RAMP1 | DHRS2 | AICDA | PRPH2 |
| TEK | FRZB | SRPK3 | HIST1H2BO | SLC51A |
| GPANK1 | MYL6 | F11 | TMEM9B | BMP5 |
| ZNF517 | Hdac7 | SH2D2A | ZNF830 | ZNF655 |
| CYB5A | NKAIN4 | MAN1B1 | CASC2 | FAM171B |
| GMFG | CLEC12A | TNNC2 | FKBP2 | GSTA1 |
| CDK7 | TAF6L | SLC26A1 | NO_MATCH_23 | KLK12 |
| BTN2A1 | TNFRSF1B | TSR1 | MRPS14 | DVL2 |
| FAM136A | RPLP2 | ITM2B | PF4 | DCAF8L2 |
| MAP2K4 | ETHE1 | MMP2 | SLC25A47 | CNOT10 |
| EPB41L4A-AS1 | ACSM5 | OR2T33 | LRRC19 | KLHDC7B |
| C7orf49 | P4HA3 | XLOC_l2_004853 | FN3K | EVI2A |
| XLOC_001164 | TRIM65 | CRYBG2 | SLC25A29 | KANSL3 |
| OGN | DDX47 | KIF1BP | YIPF2 | XLOC_l2_000394 |
| MFAP3L | KIF26B | PRR11 | NCAPD3 | ERVK3-1 |
| BICDL2 | BIN2 | LRAT | FTH1P3 | RPS6KA6 |
| IL16 | SNX21 | FHL1 | AZU1 | UQCRC1 |
| TTI1 | CCDC9 | KCNA5 | NEK5 | ARFIP2 |
| LAP3 | CD69 | ISOC2 | RNF31 | LZTS2 |
| RAB3D | ADAM33 | Bloc1s1 | C16orf87 | RIC8B |
| LRRC8B | CHRNB1 | KRTAP19-5 | RNF146 | CCNI2 |
| TRIM39 | KRT18 | ANKRD39 | RPS15A | P4HB |
| XLOC_005923 | FAM105A | PIP5K1B | SERINC4 | SRSF6 |
| S100A14 | FSD1 | GPR31 | TACC3 | HACD2 |
| PSMB9 | SCAMP3 | SAMD4A | LYSMD3 | EPN2 |
| RNF170 | CISD1 | PKD2L2 | LMAN2 | DEPDC7 |
| PLBD1 | DERL2 | GCSAML | CD244 | RHBDF2 |
| TPRN | DMC1 | GABRD | ATP5D | PRPF40A |
| NXPE3 | HBZ | GDE1 | PSMG2 | OR2A2 |
| TRIM6-TRIM34 | NO_MATCH_183 | ZACN | OSCP1 | PCDHGA7 |
| SLC25A21 | GOLPH3L | XLOC_l2_004594 | ATP6V1F | HTATSF1 |
| HAS3 | CCR1 | COL10A1 | C20orf24 | SPATA17 |
| HBM | COQ8A | TUFT1 | DYNLT3 | MYLK2 |
| KAZN | C5orf51 | ATAD3C | TOR1AIP2 | FOXR1 |
| MPP7 | HTR3D | ABCA3 | ZNF695 | MMADHC |
| MYZAP | XLOC_013281 | RPEL1 | CCDC27 | DBNDD2 |
| LBX2-AS1 | BAG5 | PCMTD2 | SLC39A1 | LRRN3 |
| FTSJ1 | GPATCH11 | LINC01341 | FZD5 | SPAM1 |
| ENOSF1 | LRRC29 | SLC12A4 | MANSC1 | PSCA |
| SNRNP35 | RNF138 | MUM1L1 | TEX26 | CERKL |
| AGPAT2 | SEPHS2 | GEN1 | POLL | TRPC3 |
| KCNJ13 | MRPL13 | UBAP2L | XLOC_l2_003293 | CDKN1B |
| PDXDC1 | CXCR3 | DDX50 | DEUP1 | DTNBP1 |
| PANK3 | DUSP10 | CCDC81 | OARD1 | MRPS34 |
| TMEM39A | KCNJ14 | CCDC62 | CDRT15 | CCT6B |
| DIXDC1 | SLC6A1 | BCS1L | NO_MATCH_232 | ZBED5 |
| AHSP | SEC16B | C4orf47 | CH25H | ZC3H12A |
| ODF3L2 | ADK | BRAF | JADE2 | LRCH3 |
| FBXO15 | HDAC1 | AQP3 | ZNF343 | NO_MATCH_140 |
| DPP7 | HNRNPLL | DIAPH3 | OR2J2 | H2AFJ |
| CDH17 | SLC5A11 | COTL1 | AHNAK2 | FRS3 |
| DUS4L | NUDC | Sec31a | HIST1H3C | NARFL |
| PIGV | TMEM11 | NME6 | SIX1 | ELK1 |
| UCN2 | WNT7A | 4-Sep | SUB1 | SYF2 |
| EGFL7 | B4GALT4 | OR1D2 | NO_MATCH_41 | PRRC1 |
| NO_MATCH_266 | ASB11 | ZNF662 | CLIC1 | ZRANB2 |
| NME2 | RRM2B | TTLL7 | TAS2R8 | PPIL3 |
| RGS5 | KLRC1 | PSMF1 | NO_MATCH_103 | CIART |
| DCAF7 | TMBIM1 | DMWD | C5orf64 | CCT3 |
| OCIAD1 | DUSP13 | ALKBH2 | BCAM | PRNT |
| Ints11 | ACTL6B | NAT8 | USP18 | DVL1 |
| NXT2 | GCNT2 | CDV3 | PPP1R1A | WDR76 |
| POLR2E | CFAP53 | FUCA2 | SAP30BP | SYVN1 |
| STEAP1B | NAP1L3 | BAG1 | CLYBL | IP6K3 |
| NUDT16 | RNASE6 | CYP2A7 | PFDN1 | VPS41 |
| ZSCAN1 | SIGLECL1 | GLIPR1 | TMEM40 | CCDC144A |
| XLOC_l2_009790 | MCUB | LIX1 | RPL11 | MYH7B |
| NO_MATCH_264 | ARHGAP26 | RND3 | FCER1A | PPFIBP2 |
| CKMT2 | OR52L1 | TAS2R14 | C11orf68 | TEX10 |
| ARMCX5 | GPR153 | FAM27E4 | HCRTR2 | NLRP10 |
| TAS2R45 | ZNF280A | OSM | BAGE | RBM42 |
| L3MBTL4 | CARD9 | MBLAC2 | SPANXD | SERPINA7 |
| NSFL1C | SLC9A2 | MAF1 | CDK1 | CPM |
| XRCC6 | ARHGAP12 | PARP3 | HIPK3 | NO_MATCH_230 |
| ACOT12 | RRH | CPB2 | IGLL5 | SBF2 |
| SSBP1 | TRMT13 | MRPL52 | PGLYRP3 | EPM2AIP1 |
| RAB30 | ARMC3 | RAD23B | XAF1 | GALR3 |
| INGX | TCIRG1 | NO_MATCH_199 | NO_MATCH_188 | ZCCHC8 |
| WSCD2 | ANO6 | EIPR1 | TKTL2 | PURG |
| HACL1 | EXOC3 | CNKSR1 | PATL1 | WBP4 |
| CDK8 | FKBP4 | SBNO2 | WNT2 | RAB1A |
| SLC5A9 | MEGF10 | TEX19 | COG8 | KCND3 |
| CLN6 | TMEM256 | TCAP | TFRC | SETD9 |
| MMAA | TMEM222 | REXO5 | TMEM2 | FOXD4 |
| TGM3 | HMBS | FAM220A | MRPL49 | DEXI |
| RHPN1-AS1 | THBS3 | ASTE1 | C11orf72 | NLRP4 |
| GALNT6 | FIS1 | ANGPTL7 | TMEM89 | SLC9B1 |
| ZNF114 | PAEP | NSUN6 | HMGN1 | KISS1R |
| CNBD1 | LMX1A | FAM20A | OLFM1 | TIMM17B |
| COMMD10 | CCDC102B | CHMP2B | CD6 | MAPIO |
| FIGNL1 | YWHAEP7 | PRRT2 | N4BP2L2 | RassG |
| Amt | FAM234A | CYP24A1 | HIPK1 | CASP2 |
| S100A8 | MYL10 | GAS8-AS1 | GPR171 | TAF1C |
| LRRC28 | NEDD9 | CTSL | RAN | LINC01600 |
| LMO2 | TOMM7 | HBB | MTMR11 | NO_MATCH_15 |
| PROSER2 | LOXL2 | DNAJB3 | OR2C3 | OR52B4 |
| PRAF2 | OR13C5 | ARCN1 | REL | PEX13 |
| GNG3 | ZFAND6 | SSFA2 | CYTL1 | ABL2 |
| B3GALT2 | OR6N1 | ECH1 | ETV5 | ERLIN1 |
| NIPA1 | KCNG4 | SEC63 | ZNF438 | MAPK11 |
| HEATR9 | RFC1 | TMOD4 | NAGS | C8orf31 |
| PSMD10 | ADAMTS4 | XPO7 | GRM5 | XLOC_012222 |
| ABCB6 | CCL20 | MYCT1 | REM1 | FBXL20 |
| CDYL2 | PPDPF | GPAA1 | LY86 | KRIT1 |
| S100A12 | EFCAB1 | CPNE7 | NO_MATCH_136 | ZBTB10 |
| ZSCAN20 | PBLD | TRIM3 | CLEC1B | ALOX15 |
| TTC41P | CLEC11A | RMDN3 | SYT5 | CHRNB3 |
| OR2T10 | NDUFA4L2 | ANXA5 | Ube2o | SYT1 |
| TBL1Y | C6orf226 | DIMT1 | SLC27A1 | PEX5 |
| XRCC3 | EMC3-AS1 | C1QTNF9B | FSIP1 | SULF2 |
| MAB21L2 | IL23A | FXYD6 | SEC14L3 | RABEP2 |
| BOD1L2 | CNNM2 | HIST1H1A | UBTD1 | RNF111 |
| FGFRL1 | RPL23A | CD52 | OR56A1 | ACPP |
| SLC26A9 | PEF1 | JAML | ALMS1P1 | NELFE |
| RAD1 | TSSK6 | RBM4B | DAPP1 | PLEKHO2 |
| CMIP | NO_MATCH_184 | NR4A2 | LY6G6D | CLK3 |
| CNP | LEMD1 | UBE2D3 | NO_MATCH_102 | OR6C1 |
| SDR39U1 | IFT57 | SCRN3 | DSTYK | GAREM1 |
| HMGA1 | HSD3B1 | ZIC1 | AASDHPPT | NCLN |
| CYYR1 | OXT | QRICH1 | HORMAD1 | SLC52A2 |
| RNF167 | NDUFV1 | VPS52 | LRMP | ZBP1 |
| DTX3 | MRPS12 | ARSJ | TMEM87A | ADGRE1 |
| MT1B | TM4SF4 | FAM168A | DRD1 | SCTR |
| CD3D | SLC6A7 | NCR3 | DKK2 | TTC19 |
| CXorf40A | FUOM | HMMR | NO_MATCH_245 | MKL2 |
| BEX5 | FBXO48 | HDAC11 | FAM71A | PURB |
| TMEM151A | CSNK1G1 | ID2 | CDC34 | DOHH |
| LOC439933 | XLOC_008618 | ANKRD13A | XLOC_l2_000581 | NO_MATCH_244 |
| KIR3DX1 | RASSF6 | PIKFYVE | CTSE | SMURF1 |
| SUPT3H | SNX12 | MYBPHL | IYD | SGSM3 |
| A2M | NRN1L | DUSP18 | TOE1 | HTN3 |
| MRGPRG-AS1 | FBXO7 | MDFI | SERPINC1 | MOCS2 |
| DQX1 | WFDC5 | MRPL38 | UPP2 | LAIR1 |
| XLOC_013207 | OR52I2 | IPMK | KXD1 | TEX30 |
| ATP6V0D2 | TNNI3 | HAUS2 | FAM221A | REEP1 |
| ZNF726 | PRR22 | CMBL | OLFM4 | PROK2 |
| ZNF490 | RASL11B | HSPA2 | NAT10 | COPRS |
| CDKL3 | PCED1A | WNT3A | KIAA0513 | SULT1B1 |
| GABRA3 | NECTIN3 | TTC28 | LMCD1 | DHRS7B |
| MAP4K1 | PPP1R15B | EDDM3A | GRN | DNAJB14 |
| LIN7C | CCDC107 | ASB4 | FAM19A3 | TRMT1L |
| FXN | GPAT2 | GALK1 | CCDC54 | ZNF346 |
| SERINC2 | CEACAM1 | SGK494 | ANKRD50 | UNC50 |
| APOF | NO_MATCH_273 | DNAJC4 | LRRC71 | ARMCX3 |
| HS6ST2 | CRACR2B | SPATA1 | SPSB1 | XLOC_009028 |
| TMEM126B | AVPR1B | SRPRA | MPP3 | MYLK3 |
| BUD23 | MUM1 | TMEM211 | CSGALNACT2 | NFIX |
| TTLL10 | TGIF2LY | ELOVL1 | NO_MATCH_258 | GRM6 |
| LARP7 | C8orf49 | ATP6V0B | URM1 | C19orf66 |
| VPS33B | SLC31A2 | NFAM1 | FAM98C | MORF4L1 |
| CYP27B1 | AIMP2 | TM4SF19 | LOC440570 | PANK4 |
| HTR1D | PPP4R4 | MAPK8IP2 | ARSA | PRAM1 |
| RAD9B | PTGDS | OXCT2 | IGFBP7 | PELI2 |
| NAA30 | RPN2 | GLUD1 | HLX | CAPN2 |
| DHX30 | SH3GL2 | ISL1 | CCL21 | CLASP2 |
| LINC01620 | ASTN2 | TMEM107 | CYP8B1 | TRIAP1 |
| DNAJC14 | SLC25A38 | TPRG1 | GABARAPL2 | PPP1R18 |
| MYL5 | CGA | MTHFSD | IFIT1 | NO_MATCH_8 |
| NO_MATCH_206 | HRH4 | SHMT1 | C2CD2 | EPB41L3 |
| EDIL3 | MCRS1 | IFT20 | SCGB1D1 | OAS1 |
| FNDC5 | IGFBP4 | GPX1 | VPS29 | KBTBD4 |
| BDH1 | CLASRP | KIF25-AS1 | EPB41L4A | ACAT1 |
| GPR37L1 | ZNF653 | ORC6 | TRIM74 | ERVW-1 |
| GNG12 | AMBP | C3orf14 | MEPCE | C1QTNF9 |
| RNASEH2C | LINC01547 | A1CF | ESS2 | TXLNGY |
| HIST1H1E | PRKAG1 | MPIG6B | COX5B | ADRB3 |
| SCMH1 | SEC11A | TXNRD2 | CD276 | DAB2 |
| RNH1 | RBP2 | SPC24 | SLC48A1 | PHOSPHO1 |
| HAT1 | IHH | CDIP1 | BPIFB1 | DSCR3 |
| TMEM14A | Churc1 | PRR30 | HIST2H3C | NOTUM |
| ZADH2 | NO_MATCH_51 | METTL15 | HADHA | ESRRB |
| Rpl3 | STAT5B | DNAJC5B | DLD | AMPH |
| HIST1H4L | DAO | BRAT1 | Prpf39 | GGTLC2 |
| MXD4 | LIG3 | OGFOD1 | STX12 | SH3GL3 |
| PDCL2 | CALM3 | JADE1 | ADGRF5 | INPP5E |
| SMDT1 | RAE1 | FRYL | NO_MATCH_149 | MAP4K3 |
| CLK2 | ATP5B | SDHAF3 | SMPX | ADAMTS15 |
| NUP210P1 | RLN3 | HLA-DRB5 | MPI | RAB9A |
| PDE6H | C5orf49 | SPIC | FAM209A | FZD3 |
| HGFAC | FANCC | DPP4 | SUCLG2 | P2RY8 |
| LINC00671 | CHCHD7 | HIST1H2BH | FOXP1 | SRPRB |
| LOC105369201 | MDH1 | HSD3B7 | MPV17 | SERPINA10 |
| YIPF3 | XLOC_013189 | SYCN | ORMDL3 | UHRF1BP1L |
| MFSD14C | BAIAP2L2 | MGC34796 | RASL12 | BMP4 |
| SIGMAR1 | MMACHC | SLC25A18 | CX3CL1 | SFN |
| CCL8 | DCAF8 | C1orf174 | PTK2B | CCL18 |
| GNMT | CYP4A22 | OR6B2 | TAS2R1 | ANKRD10 |
| IL2RG | KCNK4 | AOAH | LPIN3 | ZNF610 |
| STX11 | MGC70870 | PAF1 | KIAA0391 | ALOX5AP |
| MTERF2 | ADH5 | CKMT1A | BBX | MX1 |
| LPAR6 | ABRACL | ZNF524 | EXOSC1 | HK2 |
| OVGP1 | PHYHIPL | HTR3C | HSPH1 | AATF |
| SYCE3 | KCNJ15 | LINC02370 | NAE1 | FUBP3 |
| CNOT3 | INSL6 | C1QTNF3 | FGF22 | IPCEF1 |
| NO_MATCH_25 | SLC19A2 | OPTN | SAT1 | SYNCRIP |
| GNB2 | KPNA3 | LPCAT2 | CXCL11 | NPY6R |
| EMG1 | FGF10 | VASH2 | CCDC43 | TINF2 |
| TSNAX | RPS6KB1 | AP1S2 | UCP2 | EFCAB14 |
| NIT2 | CSF3 | DNAJA3 | PPEF1 | TMEM214 |
| STX10 | RNASE2 | TBC1D22A | ADPGK | NDUFA4 |
| ACKR3 | SPATA3 | WIPI2 | PROC | CABP1 |
| NO_MATCH_185 | EIF3D | NSMCE3 | LOC107984064 | ZMYND8 |
| FDX1L | RETREG1 | PXK | CCNDBP1 | CSGALNACT1 |
| CALCB | STK24 | TBX20 | MCEE | PSMD6 |
| LAPTM5 | HTR4 | LACRT | 7-Sep | RBMS3 |
| SS18 | YRDC | FAM57A | ZPBP2 | CELF5 |
| ATP5L | PHYHD1 | FAM207A | LSP1 | ATP1B3 |
| KLRG2 | IFT22 | ZNF738 | CCDC103 | PEAK1 |
| PDIA6 | PMVK | RFC4 | RPS4Y1 | TMEM38B |
| DECR2 | KIR3DL2 | CETN3 | NO_MATCH_104 | LINC00597 |
| MS4A15 | DTWD2 | TBCC | ETFBKMT | GFM2 |
| C9orf24 | LRFN3 | TWF2 | STPG1 | ATP1A3 |
| GBP3 | XLOC_l2_014804 | C1orf94 | PEMT | CDCA8 |
| PRKAB1 | LOC107986912 | SNX32 | ZNF83 | NO_MATCH_170 |
| CDH23 | KIAA1683 | FAM102A | PIK3IP1 | CLDND2 |
| ICAM2 | ARPC3 | ERMP1 | UGT3A1 | USP14 |
| Tardbp | DOK1 | GUK1 | POLR2J | ATP2B2 |
| BLVRA | XLOC_l2_013503 | TBC1D25 | TMCO6 | LRRC4C |
| ST8SIA4 | FAM227B | POC1A | RPL5 | IHEM4 |
| LOC100101148 | PHF10 | NINJ2 | PPM1M | POLR3K |
| SERPINA5 | COL4A3BP | XLOC_005602 | IL1R2 | RPS13 |
| STAT6 | LRSAM1 | PANK2 | PTK7 | ZNF23 |
| NANS | DNAJC12 | RNF213 | AOC2 | NHSL2 |
| EIF2B1 | RABGAP1L | MAPK9 | TREX1 | IGFN1 |
| BTBD16 | CXCL2 | NO_MATCH_63 | RTL4 | COMMD5 |
| COX5A | SCP2 | XLOC_000477 | KLHL28 | EEF1E1 |
| WDR46 | DEGS2 | INO80C | ORC4 | OR8D4 |
| AARD | FYCO1 | RPL18A | ST8SIA3 | SSC5D |
| SCGB1C2 | CRH | STEAP4 | RSG1 | GAS7 |
| ANKRD49 | LDHAL6B | ID1 | MRI1 | NO_MATCH_4 |
| PSEN1 | SYT9 | CAPN1 | NMUR1 | SIN3A |
| RACK1 | SLC35A2 | DDR2 | VPS37B | NFE2L1 |
| PGK1 | KHK | AKTIP | ADRM1 | SLC36A3 |
| CTNNA1 | CD46 | PMPCA | HSPA8 | WDR34 |
| S100A6 | SIVA1 | INPP5J | RIMS3 | POC1B |
| RUFY1 | GOLGA7B | TXNDC8 | XLOC_l2_015213 | AKR7A3 |
| SPEG | CDKN2C | GJB5 | TAF8 | EXOC5 |
| MMP10 | TBC1D3H | MAP2K2 | PSMD13 | KRTAP9-8 |
| MYDGF | HTATIP2 | HOPX | SERPINB4 | MAP7D2 |
| OPALIN | RNF148 | DFFA | LOC541473 | C1orf131 |
| CD96 | FKBP6 | AMIGO1 | SPRED1 | TRIM49 |
| ACTG1P17 | ADAL | POLR3C | AVPR1A | MAN2B2 |
| XLOC_005712 | MED4 | ANGPT2 | PICALM | RPL19 |
| WDR45 | SIX4 | TPT1 | RASD2 | RANBP10 |
| ACTL7B | PSMD9 | DUSP23 | SAYSD1 | ZNF800 |
| ZBTB1 | KNOP1 | COLEC10 | YPEL4 | ZNF77 |
| RAD54L2 | SELENOH | RECK | SLC4A8 | MBD3L1 |
| NO_MATCH_43 | FRA10AC1 | AMMECR1L | RBAKDN | C7orf25 |
| FAM96A | PFN4 | VBP1 | SLC22A24 | ECSCR |
| KCNE1 | FLT1 | IFI27L1 | FBXL2 | IFT74 |
| TMEM86A | MIR650 | CEBPG | GABARAPL1 | C17orf80 |
| ETFB | RPL3 | TACO1 | SYT3 | DPF1 |
| DNAJC10 | COG4 | TMED6 | SSX3 | PALLD |
| PLPP2 | NDP | NSL1 | ASPHD2 | PRTFDC1 |
| RALB | HSPA12B | C6orf89 | ZCCHC12 | ICOS |
| IFT80 | ABRAXAS2 | EML1 | ADAMTS5 | DIS3L |
| ADGRB2 | MEN1 | FKBPL | DHX40 | MIA2 |
| NO_MATCH_246 | FMO9P | TMEM51 | LOC644936 | SLC37A2 |
| ADH4 | SAMD11 | NO_MATCH_66 | EDEM2 | DNASE1L3 |
| HYAL2 | ANKRD55 | LOC254896 | KRTAP10-9 | GPR18 |
| CCDC50 | NDUFB11 | MSMB | RAB11FIP4 | CRY2 |
| NIPSNAP2 | LYRM1 | COA7 | EPB41L2 | CYP51A1 |
| ACOT1 | SLC7A8 | GNLY | TNFAIP6 | CHCHD4 |
| WDPCP | C4orf26 | WDR83OS | CDKL4 | SLC6A13 |
| NEK3 | C7orf69 | RFX2 | GFRA1 | LOC441242 |
| PPP1R27 | RPS6KA5 | COPZ1 | AP3B2 | TFIP11 |
| LRRC8E | ZNF230 | LAT | RPP21 | DDIT4 |
| GAB2 | AGGF1 | CDH26 | CXorf40B | MDM1 |
| HOXD9 | OR5F1 | TMPRSS4 | HHLA3 | SAMD3 |
| CHST15 | GP9 | S100A1 | SV2B | GNGT1 |
| SCGB2A2 | SNX1 | NO_MATCH_241 | PTTG2 | MIR17HG |
| HSF1 | NO_MATCH_208 | RAB24 | ADGRF1 | MRPL18 |
| PSMB8 | SDSL | L3MBTL2 | SLC46A1 | ERCC6L2 |
| THRA | THUMPD2 | EDNRB | CCDC115 | CDK18 |
| CCDC142 | TMEM88 | LOC100506127 | CST9 | CSNK1D |
| DNPH1 | ASMTL-AS1 | XPC | SLC25A17 | C22orf34 |
| ANKRD22 | PRSS2 | ACTRT2 | PCDHGB4 | CCDC91 |
| ST3GAL2 | CGB8 | LNX1 | FBXO21 | CHRM4 |
| NETO2 | IL33 | PGLYRP4 | FXYD3 | EIF2AK2 |
| Prrc2b | SRD5A3 | TOP3B | CYSLTR2 | C17orf102 |
| TMEM147 | OR1E2 | GSG1 | H2AFB1 | TMEM62 |
| HMGN3 | FAM200A | TCF3 | NPFF | LRRC32 |
| TAS2R41 | APPL2 | AREG | LRRTM2 | KIR2DL4 |
| BPI | BMPR1B | CXCL3 | HCFC1R1 | FGR |
| DSN1 | GPRC5D | CBY1 | CSTF3 | OXER1 |
| UBE2Q1 | OCLN | PDE1A | RWDD3 | ZNF461 |
| SPINK4 | CCDC74A | PRMT8 | SNAPC3 | CCDC12 |
| ZFP91 | LIM2 | XRCC2 | CNIH4 | GCA |
| SMPD2 | C17orf67 | CD209 | FAM167A | FAM222A |
| FDXACB1 | MTFMT | XLOC_l2_007835 | IL22RA1 | DMTN |
| TCL1B | CRIM1 | ZWINT | GPRC5C | AFG3L2 |
| RC3H2 | DRC3 | RNPC3 | MORC3 | UGP2 |
| CCL16 | GALNT15 | TGM5 | ADAM2 | RUVBL2 |
| KLHDC9 | SGK1 | RIPPLY2 | EXOG | RAB3GAP1 |
| OR1Q1 | CCL5 | NO_MATCH_209 | GRM7 | KRTAP19-4 |
| FAM122B | GPR142 | NTMT1 | CELF4 | HELLS |
| PPARGC1B | DARS | LSM6 | RNF151 | ACADM |
| ACTA1 | GALNT7 | OR2T2 | PTGER2 | OPN1MW2 |
| Adamtsl1 | FAM86C1 | TAAR1 | XLOC_002741 | MTM1 |
| STATH | PILRA | SLC34A1 | C9orf78 | NOL4 |
| WDR74 | TXN2 | DCTN5 | LGR6 | CEP44 |
| ZNF264 | GABRE | AGO1 | PTAFR | MRPL50 |
| VCAN | MRPL9 | CCND2 | FCER2 | TCTE1 |
| SSTR1 | LOC107984344 | ZNF707 | NO_MATCH_34 | ZFY |
| PATZ1 | NO_MATCH_72 | FAM72B | Fam76b | NKD1 |
| RNF34 | LINS1 | NPY1R | TMEM108 | MCEMP1 |
| VPS11 | EHD1 | RPL9 | IFNE | ALPPL2 |
| SPATA2L | RAB40AL | PSMD2 | ZNF418 | CAGE1 |
| CSRNP2 | UBA2 | IL2 | ACBD5 | CRNN |
| CADM1 | CALM2 | RUVBL1 | ZNF511 | SPATA46 |
| TMSB4XP8 | LMNB1 | YTHDF3 | CKMT1B | CKAP2L |
| ADM | HNRNPA1 | SMO | QPRT | CYTIP |
| FCGR3A | TPST2 | UBXN2A | 5-Mar | TIGD3 |
| PFN1 | SERPINB9 | MAPKAP1 | CD274 | ADGRF3 |
| MINDY3 | CLDN7 | BRD3 | ETAA1 | KCNK6 |
| LTK | EYA3 | KCNMA1 | ASNS | TAAR6 |
| CENPT | AP1S1 | ST6GAL2 | PDGFRA | STRC |
| SPATA24 | NO_MATCH_254 | NO_MATCH_70 | CTNNBIP1 | VIPAS39 |
| RPL10A | COL9A1 | POU3F2 | UBL4B | TXLNB |
| PHB2 | GAGE12B | ATP1B1 | GRB2 | MASP1 |
| ATPAF2 | PFKL | UFM1 | GNA11 | E2F6 |
| CEP70 | SCGB1D2 | MYBL1 | LINGO1 | SLC10A2 |
| TMEM129 | TMEM86B | CCT5 | PPFIA4 | CEP126 |
| CHKA | SORBS1 | NO_MATCH_195 | AANAT | OPN5 |
| NANP | TSSK4 | ZNF586 | RGS11 | GPR34 |
| MRPL47 | HRH3 | PRPF4 | KCND1 | CGGBP1 |
| MPND | KRTAP13-4 | NO_MATCH_111 | EZR | CELF6 |
| MAB21L3 | FAM58A | RNASE9 | CLK4 | MRPL16 |
| PGC | VPS37A | RPL18 | CYP2C9 | C11orf84 |
| SLC29A3 | ZNF497 | LINC00471 | CCDC185 | VPS35 |
| AVP | TOMM5 | KIR3DL1 | MS4A3 | ING3 |
| PCP4 | POLR2H | GULP1 | PRKCZ | FAAH2 |
| HSP90AB1 | RGN | PHF19 | ACTN4 | PRF1 |
| PID1 | VTCN1 | CRYGD | TCF19 | BORCS7 |
| RIMBP3 | MECOM | LINC00312 | OR6C65 | TEKT1 |
| EIF5A | LEXM | ZNF414 | PPCS | BRSK2 |
| CPSF4L | GOLT1A | RPH3A | FOXS1 | DYNLL2 |
| SLC25A27 | MFAP3 | HAL | RBM3 | ANOS1 |
| MLLT3 | CLDN9 | SLC45A2 | LIPF | RPS18 |
| NO_MATCH_247 | UBAC2 | TMEM72 | PHPT1 | KLHL14 |
| CCDC197 | ODF4 | ARIH2 | AMZ1 | JPH1 |
| NO_MATCH_229 | NINJ1 | AASDH | GPR1 | SEZ6L2 |
| CYP19A1 | DSCR9 | PNPLA4 | TERF2IP | KCND2 |
| ROM1 | ADH7 | HP1BP3 | KIFC1 | DOK2 |
| CISD2 | CDC123 | PSMB7 | YTHDF2 | MAPKAPK5 |
| PFDN5 | MMP15 | CASP8 | XLOCO_10930 | TACR1 |
| FAM212B | HSPB8 | SPATS2L | GRIN2B | SRGAP1 |
| PAQR8 | NNMT | TMEM167B | LGALS14 | DTNB |
| PRKAA1 | TPH1 | SEC11C | APOA1 | CXCR1 |
| TRDN | KLK15 | ST6GALNAC4 | REEP6 | GP1BA |
| SLC39A11 | GCC2-AS1 | PRKCSH | PCOLCE2 | MRPL34 |
| OCIAD2 | INPP4B | LOC105379861 | KCNC4 | TM4SF5 |
| GPIHBP1 | STYK1 | MRPS18A | NRIP3 | CDH5 |
| ATG9A | FABP12 | NO_MATCH_159 | ATP6V0A2 | ECHDC3 |
| HINT1 | SFXN5 | FASTKD3 | EPC1 | PSMD7 |
| UTP3 | NFKBIB | C1D | GPR65 | NAPG |
| SAMHD1 | NELFA | ALKAL1 | R3HDML | ACD |
| RPRD1A | ASB10 | NO_MATCH_248 | VMP1 | DTX2 |
| NO_MATCH_123 | C19orf54 | BOC | MAGEB10 | ZNF512B |
| SERPINA3 | IQCC | TBC1D23 | TARSL2 | ARHGAP5 |
| PRKCE | DPYD | LHFPL1 | AK3 | FASTKD5 |
| SMUG1 | INPP4A | C1orf109 | CCDC105 | CBLC |
| MTHFD1L | GABBR2 | CDKAL1 | EGFL8 | ZNF213 |
| MYLK | RIPK3 | FAM218A | HEXIM1 | KRT72 |
| SELENBP1 | CCDC196 | NO_MATCH_252 | DIEXF | PEPD |
| SUV39H2 | HFE2 | MAPKAPK2 | DNAJC7 | CCNH |
| UPRT | PEX11A | CPLX1 | PPA2 | C1GALT1C1 |
| RGS20 | MRPL17 | XLOCO_10007 | LPCAT1 | EIF2B4 |
| GADD45G | C1orf226 | PPTC7 | THPO | PNLIPRP3 |
| TGM2 | KLC2 | DDX39A | EIF3L | GAS2L1 |
| CRCP | MSR1 | GLYATL2 | TRMT10B | HAPLN2 |
| OSBPL11 | CRYBAI | TNP1 | DOK6 | RPS3 |
| IMPACT | OR9Q1 | KIR3DS1 | MT1X | FGD5 |
| PLAC9 | ZNF132 | PLD5 | SERPINB6 | LDHD |
| HMGB1 | PER1 | CLEC7A | FGF8 | GNRHR |
| NME1 | SYT14 | ACADSB | EIF1AD | PTGR2 |
| PIANP | HIST1H4J | SDCCAG3 | CCR8 | C8orf59 |
| SNRNP40 | FAXC | RBM34 | LAMTOR4 | SH2D4A |
| PFDN4 | PIWIL1 | TXNDC15 | CAMLG | PRND |
| TUBAL3 | TMEM55B | ARNTL | HBQ1 | VN1R10P |
| GLMN | ABHD12 | GMNN | CYP20A1 | NO_MATCH_249 |
| TMEM133 | ZFP57 | FBLN5 | STAU2 | NISCH |
| PDPK1 | ARHGEF19 | PABPC5 | PSMD8 | FLOT2 |
| CSN1S1 | C11orf87 | ZFP37 | PLPBP | KIF22 |
| MGAT5B | SLC35F3 | ADRA1A | PEX10 | PTPRA |
| CKM | STAMBPL1 | NO_MATCH_162 | EEF2K | PTGDR |
| FAAP100 | NOG | COMMD2 | FAM208B | KARS |
| DUSP12 | ATXN3 | RNF24 | BUD31 | FAM192A |
| PLAT | ATP13A4 | SIRT6 | TAS2R50 | KIF6 |
| MET | ITPKB | CCDC146 | SURF6 | HLA-E |
| MAP3K13 | NAT16 | ETFRF1 | 2-Mar | SLC1A1 |
| C5orf58 | KCNH6 | BCL2L13 | HIST2H2BF | MOG |
| CST5 | C17orf75 | DBH | PRM1 | POU6F1 |
| ZMYND11 | BTLA | HIST1H3E | ZFAND2A | CCK |
| HTR7 | POMGNT2 | TNFAIP1 | OR11H4 | CRTC3 |
| CST1 | AK8 | LENEP | CRYGC | ATP5G1 |
| EIF3I | IRAK1BP1 | CRYGB | PRAMEF15 | GADD45GIP1 |
| SNCA | PGRMC2 | LOC102724993 | RABGEF1 | DDX18 |
| ZNF761 | C11orf53 | GABRB2 | ABI1 | CYBB |
| CDK2 | TBC1D26 | Fgd6 | CA9 | AP1G2 |
| GABPB1 | SLC17A2 | SLC22A13 | DPYSL3 | LGALS9 |
| ILK | CA5A | MOB1A | GINS1 | CAVIN1 |
| OR56B4 | PYY | ANTXR2 | IRF3 | ZNF320 |
| HIST1H4H | CATSPER4 | HLA-DRB4 | NO_MATCH_270 | DNAJB1 |
| TRAF3IP1 | NT5C | NO_MATCH_221 | KCNK1 | LGR4 |
| FMO2 | NO_MATCH_90 | USP10 | OR1D4 | SYNDIG1 |
| CDKN2D | CLSTN3 | CAPS | RBM10 | PLCD4 |
| SOX3 | LGALS9C | EIF4E3 | CLHC1 | SEC61G |
| ASB13 | POLG2 | CLIC3 | HIST1H2BN | AGRN |
| GPR19 | TCEAL5 | BMI1 | INTS5 | CTNND1 |
| GAGE1 | TAL2 | SFXN1 | MGST3 | RITA1 |
| FAAP24 | SPATA22 | CRISPLD1 | STARD7 | ZNF334 |
| DPPA4 | CADPS | Egln2 | NXT1 | DPYSL4 |
| DCAF5 | AES | VAMP1 | GPKOW | MACC1 |
| ELOVL7 | FAM60A | SMIM19 | NO_MATCH_153 | C18orf25 |
| DESI2 | RASGEF1A | MAPK13 | KIAA1551 | NOXO1 |
| SNX4 | ADAD2 | ATP6V1C1 | U2SURP | DHCR24 |
| COMMD6 | MBP | SRP54 | GMPPB | ADARB1 |
| CTAG1A | AP2S1 | SLC2A9 | PAK6 | NO_MATCH_47 |
| STK33 | LIPM | TDRD10 | ZKSCAN5 | TAZ |
| CRABP2 | UTS2R | RFC5 | RCSD1 | PKN2 |
| PCNX2 | LECT2 | CNNM3 | MAPKAPK3 | GJB6 |
| TFR2 | KIF26A | ERI2 | SYCP3 | NPBWR1 |
| GSTK1 | NO_MATCH_177 | IL17F | C12orf43 | TBC1D21 |
| FAM106CP | TRAP1 | ARMT1 | GSDMD | ZNF488 |
| NPHS2 | NPRL2 | DBR1 | TNFAIP8 | UFL1 |
| KDELR2 | ARHGAP27P1 | XLOC_l2_009281 | TUT1 | ZNF35 |
| HIST1H4I | NO_MATCH_{——}137 | CHCHD2 | FABP2 | FBXO24 |
| ZNF608 | PTTG1 | NCK2 | MAP1LC3A | RPN1 |
| ZNF19 | TPO | RBM4 | NO_MATCH_32 | MTMR14 |
| ZFP36 | FAM216A | DIDO1 | PLPP6 | MT1A |
| EIF2S3 | RAB4A | TUBA4A | TIAL1 | ADAMTS1 |
| SLC28A3 | RSBN1L | TMED7 | ACOT4 | MTFR2 |
| POLR1D | NO_MATCH_5 | SMR3A | RPL15 | INHBA |
| LRP2BP | GTPBP2 | CLEC3B | KLK11 | CYP1B1 |
| FAM204A | DBNL | CFHR3 | IL31 | OR52B2 |
| OR1M1 | TMEM102 | HSPA14 | ARNT2 | KLF10 |
| FBXO8 | OXLD1 | SPHAR | F2R | SNX8 |
| HUNK | STX5 | OR5C1 | SLC3A2 | TMEM68 |
| VAMP3 | SELENOM | ATOH7 | TAS2R10 | PZP |
| ART1 | RPSA | ZNF146 | NO_MATCH_88 | 10-Sep |
| DYNC2LI1 | MIF | Tmem208 | HAGHL | UGT2A3 |
| GPR176 | SVOPL | FN1 | VGLL3 | JMJD6 |
| DRG1 | LGI4 | CAB | CYP46A1 | XLOC_004269 |
| IFI27L2 | RBM18 | RORC | ZNF79 | EIF4A1 |
| MIEF2 | C6orf120 | NME3 | PCDHGC4 | TSPAN5 |
| HLF | Rp136a | TESK1 | HS1BP3 | RGS2 |
| THNSL1 | ABHD14A | KERA | WFIKKN1 | RPL8 |
| LINC00314 | TRIM61 | PSG6 | C14orf28 | XLOC_l2_006804 |
| FKBP11 | MRPL1 | HIST2H2AC | GAL3ST2 | GBX2 |
| TM7SF2 | NO_MATCH_235 | MAPK3 | C8orf22 | AGFG1 |
| UBAC1 | CHST8 | ELK4 | ZNFB8 | SETX |
| FABP1 | HES1 | ZXDC | PTER | C14orf37 |
| NIPSNAP3B | UQCRQ | ZNF616 | CCDC110 | SHTN1 |
| CAMK2A | TYW5 | LOC100B0950 | PPM1B | NO_MATCH_213 |
| KCNA3 | ANKEF1 | CDNF | LIMS3 | CDH16 |
| CXCL1 | NO_MATCH_267 | FUZ | METTL14 | KRTAP13-3 |
| DSCR8 | NOL12 | TMEM63C | ZNHIT1 | FBP2 |
| LPL | PPM1L | MRO | MRPL32 | NTF4 |
| DGUOK | OS9 | AMTN | MOS | ABHDB |
| TSPYL6 | C7orf33 | SELENOK | MAP3K15 | COMP |
| VSTM2A | RGS13 | NO_MATCH_59 | MRGPRF | RERG |
| LRRC15 | GHRL | CENPX | MIR4697HG | TMEM140 |
| SLCO4A1 | NREP | AHCY | LINC01667 | MXRA8 |
| CTSZ | CELA2B | LINC00467 | AZGP1 | SSTR5 |
| PSKH2 | VASH1 | ARMC7 | SGK2 | EPHB2 |
| CCDC102A | ERG28 | COX14 | LRRC37A | GLP1R |
| GINS3 | NAPA | SPATA4 | C20orf141 | NKAPL |
| NOXA1 | STARD7-AS1 | GAS2 | TSPAN2 | HHATL |
| CHRNA6 | KCTD15 | BTBD6 | TCHHL1 | OTUD6B |
| SELENOS | IL21 | RPRM | PCDHGB5 | PTH2R |
| MAP3K19 | RRAD | C11orf49 | NSMCE4A | HOXC11 |
| RNF41 | POLD4 | ADPRHL2 | SNRPB2 | ARSE |
| KRT6B | SLC43A2 | AP5S1 | HSD11B1L | POLR1B |
| TRIM51 | BRMS1 | CHRFAM7A | C17orf47 | MPEG1 |
| GABRR2 | NOP10 | SNX24 | RELL1 | ARHGEF7 |
| ELMOD2 | GPC4 | THYN1 | SPDEF | RBMX |
| CIR1 | RAB27B | GPLD1 | C21orf91 | PATE1 |
| ADAM17 | TRIM29 | TBXAS1 | PLA2G4D | CPNE9 |
| TAF1B | VKORC1L1 | LINGO4 | SURF2 | PRKD1 |
| NFATC2IP | CER1 | TAGLN2 | BIRC5 | TMEM92 |
| EMC3 | Atp11b | CHRM2 | PPP1R13L | RAF1 |
| OXSM | ZNF587 | SLC38A1 | LINC00638 | RAD21 |
| PLPP5 | DPPA2 | ANKRD16 | ZNF295-AS1 | APBA3 |
| NAT2 | GLT8D1 | KRTAP23-1 | GBP7 | PDE3A |
| GNG4 | INVS | CEP95 | LOC107987253 | SLC45A3 |
| HLA-DPB2 | MRPS24 | ABHD15 | VPS28 | HK3 |
| KLHL12 | RPL35A | CCDC59 | ARHGAP18 | KANSL1 |
| HOXB5 | STK25 | C2orf40 | MCCD1 | WDR44 |
| AIFM3 | CA10 | FUNDC2 | OPN3 | NRXN3 |
| ADGRG3 | ALDH3B1 | SPAST | TNFSF8 | HTR1E |
| CHPF2 | NSMCE1 | RHOD | XLOC_010651 | CCDC86 |
| LIME1 | NUPR1 | HIPK2 | OR1A1 | MGRN1 |
| IGFBP2 | DKC1 | DHRS11 | NDUFV3 | FGF3 |
| TMEM177 | RPS29 | RHO | RHPN1 | XKRX |
| C10orf53 | ZBTB18 | SLC6A15 | ARFRP1 | COL2A1 |
| TIMP1 | DDI2 | RGMB | PHF1 | RAB25 |
| YWHAG | SLC22A1 | FBXL3 | Timm8b | WNT5A |
| CYP4F11 | TCL1A | ACVR1B | CD160 | MICA |
| KATNAL2 | REG4 | MKNK2 | MAN2C1 | KCNAB1 |
| CACNG5 | DEFA4 | LINC01270 | ANXA11 | C12orf60 |
| HS3ST2 | DECR1 | RANBP3L | ZNF44 | SLC25A14 |
| MTCH1 | FLT4 | MCHR1 | TMEM39B | ATP6V1C2 |
| STRBP | OR13A1 | GPX3 | EFCAB11 | ACO1 |
| CYP2B6 | ANXA2 | NUP210 | VARS | ICAM1 |
| CLEC2D | SLC22A17 | HS2ST1 | PPIF | TXNIP |
| PPP1R3C | TMEM135 | HPCAL4 | GKAP1 | OGFR |
| UBE2K | CACNG4 | TRNT1 | LMOD3 | HEXA |
| TSSC4 | IKBKB | SH3BGRL | NDUFA1 | CCDC158 |
| RPL23AP64 | CD2 | LOC105372824 | PIFO | SPERT |
| SLC16A13 | ODF3L1 | FAM229B | CDC5L | NTM |
| TMEM95 | ZNF225 | PKMYT1 | COA6 | NID2 |
| LINC01312 | CELA3A | PIGP | RELL2 | C5AR1 |
| AP1M2 | ERC1 | SRP9 | RBM5 | GPR37 |
| Fbrs | CORO1B | TMEM37 | CLDN10 | SETMAR |
| BOD1L1 | MTUS1 | CEP57 | STIM2 | RBM12 |
| STAM | RNFT1 | OR52K2 | KRTAP19-1 | ELOVL3 |
| UXT | MYL12A | KLK1 | NUP85 | ZFAND4 |
| GMIP | WFDC11 | UBA5 | VASP | LARS2 |
| FAM9B | NDUFAF8 | IL1RN | MAGED1 | OXCT1 |
| UMODL1-AS1 | UCK2 | FGFBP1 | HHEX | PLAUR |
| BACH1 | LBP | ASF1B | SLC10A1 | NARS |
| RERGL | HOMER2 | SH3RF2 | CELA3B | GIT2 |
| AMDHD1 | CLEC19A | NO_MATCH_142 | ARV1 | TAS2R42 |
| MRM3 | CDCA4 | NEU4 | CAI | SIX2 |
| ZAP70 | GLRX3 | GLRA1 | AQP5 | CCAR2 |
| TEX37 | TRIM59 | TIGIT | SCGB1D4 | STXBP1 |
| B3GALT5-AS1 | MIER1 | CNDP1 | PHYH | FGF9 |
| SAA2 | TMEM254 | LIMK2 | STARD3 | CDY2A |
| SET | NO_MATCH_128 | PFKFB3 | DYRK1B | PRKN |
| LTA | FAM53C | CERCAM | RRAGA | ZFYVE27 |
| ZNF333 | ZNF593 | UBA52 | SORCS1 | S1PR4 |
| ABCG8 | OR51B5 | GSTM4 | SREK1 | GATS |
| 11-Sep | PAGE4 | CRP | CXCL5 | REXO4 |
| NO_MATCH_219 | SLC36A2 | CST8 | SNUB | ARHGAP20 |
| HRG | ASTL | DEDD2 | METTL21C | CCDC47 |
| GPR82 | POLDIP2 | SOAT1 | ZRSR2 | RAB11FIP1 |
| GXYLT1 | C6orf203 | FAM107B | MAGEA5 | PER3 |
| LMBRD1 | TAC4 | CUTC | PPIH | SPX |
| Osbpl6 | TEX33 | IGSF6 | SLC2A5 | FRMD8 |
| CCL3L1 | ALDOB | ARL15 | XRCC4 | NSMAF |
| XLOC_007668 | OR8A1 | S100A9 | PLK3 | Mchrl |
| XPO5 | LAGE3 | MPPED2 | OPRL1 | SYCP1 |
| IL13 | DEF8 | CERS6 | PDCD2L | FBXO27 |
| RPL21 | LCN15 | AK1 | NO_MATCH_132 | PYROXD2 |
| CIAPIN1 | IMPAD1 | 12-Sep | PSMC2 | HYDIN |
| RHOG | GRIA1 | TTC32 | SARAF | PNN |
| COLGALT2 | CD300E | SPRR3 | MRPS36 | DPEP3 |
| SPATA19 | FAM177A1 | PADI3 | CACNA2D4 | PROKR2 |
| HAGH | KLF7 | PPP2R5C | PTHLH | PORCN |
| CFAP52 | SEMG1 | SNAP91 | AHCYL2 | KIFC2 |
| SWSAP1 | CCL2 | AVIL | CHRNA4 | EXOC6 |
| NEK6 | GTF2H2C | MAPRE1 | CDX1 | DNAJB4 |
| CXCL16 | SPIN4 | ZBED4 | NUCB1 | HTR1F |
| SPINT2 | LCK | SGCZ | C8orf4 | POC5 |
| CCDC106 | KHDC1 | ZDHHC6 | DDX53 | NO_MATCH_253 |
| ACVRL1 | NSUN5 | BMP10 | GCSH | NPM2 |
| FUT3 | C20orf96 | FCMR | PRELP | CNTNAP3 |
| NO_MATCH_186 | BTG3 | SLC39A8 | MZT2A | HSF5 |
| ASNA1 | USP37 | HSPBP1 | ARHGEF40 | KIF3C |
| TRAPPC13 | RAD51C | KDELR1 | CCRL2 | HDAC8 |
| DLG5 | GPR156 | GPR85 | PCDHA4 | INO80 |
| NKG7 | IL12B | ACTRT1 | WDR88 | HACD1 |
| EMP3 | PMS1 | C14orf159 | Pds5a | ATIC |
| KBTBD2 | TCP11L2 | DHRS3 | FLJ44635 | DHRS13 |
| HMGCLL1 | MCM10 | FBXL19-AS1 | RGS9 | RGCC |
| MRPS27 | ALDOA | TCEAL1 | EMC4 | C1orfM |
| IRGM | PSMC3 | BEGAIN | GIPR | PAGE1 |
| PNCK | GUCA2B | DNAH14 | DMRTB1 | SLC5A2 |
| LSMEM2 | CHRAC1 | DIO2 | OR2A7 | SLC27A2 |
| PPARA | SNAP23 | TRAF2 | P2RY4 | WDR31 |
| PLA1A | PAK2 | NUP37 | DDR1 | CHMP1A |
| USP54 | RAPSN | RPL39L | CASR | PHLDA3 |
| OR4C5 | ATP6V1E2 | DAD1 | ARMC10 | AZIN1 |
| ZNF782 | Acss2 | DALRD3 | RAB29 | SCN2B |
| ADH1A | HCP5 | CYLC1 | BCAN | KCNH1 |
| IQCF2 | PLTP | UCK1 | ATP5A1 | THY1 |
| SERP1 | RCOR3 | KIAA1143 | CCDC71 | SOHLH1 |
| DENR | LCE1B | EXOSC3 | NXPH1 | PHF14 |
| CDK14 | BTBD10 | ZNF175 | BOLA2 | INMT |
| PI16 | ADPRH | PATE2 | CTTN | NO_MATCH_224 |
| SLC25A3 | DDX27 | MIS18BP1 | TMEM121 | METTL17 |
| NO_MATCH_21 | MAPK14 | USE1 | REEP4 | VLDLR |
| RGS4 | IFIT3 | MTERF4 | CSRNP1 | C19orf25 |
| RAB7A | AKR1C1 | NO_MATCH_243 | CCDC92 | ADGRE5 |
| NO_MATCH_216 | SLC35E1 | GPR161 | RBM28 | SERPINB2 |
| NO_MATCH_218 | SLC16A11 | NR4A1 | RPL26 | C5AR2 |
| TARBP2 | TRIM16L | SLC39A2 | TTLL1 | KRT4 |
| HNRNPCL2 | FOXP4 | C11orf52 | SCG2 | MCF2L2 |
| NEK9 | PCSK1 | TMPRSS6 | ROR2 | SUGCT |
| HIST1H2BM | ASF1A | KLRF1 | PBXIP1 | SMYD4 |
| ALKBH3 | TNFSF11 | CCDC127 | NUBPL | NKAP |
| OR2B2 | OR5B12 | FABP9 | RPS6KA2 | RGS6 |
| EFNA1 | ATPIF1 | PAQR6 | S100A4 | NAPSA |
| CHORDC1 | INSRR | IGFBP6 | FBXL5 | OR4D6 |
| RCHY1 | REG1B | NO_MATCH_48 | PPP2CB | MRFAP1 |
| NO_MATCH_33 | SASS6 | PLA2G16 | FERD3L | ZCCHC13 |
| SYS1 | PRPSAP2 | MOK | IL13RA2 | LRRC25 |
| SMCO3 | CDYL | CD2BP2 | LAMTOR5 | U2AF1 |
| MPC1 | CASC1 | RFX3 | UBE2B | SUZ12 |
| KRTAP3-2 | GPR22 | PRSS30P | TNFRSF14 | LYPD4 |
| WDCP | CDC20B | KRT86 | OPRK1 | NO_MATCH_171 |
| SLC27A4 | HERPUD2 | MCHR2 | MEA1 | SNRPA |
| SLC13A2 | TMEM184A | WFDC6 | FFAR4 | SLC22A6 |
| NPRL3 | AQP10 | RAC1 | SGCA | SLC10A6 |
| BIK | DNAL4 | ARPP21 | NEUROD4 | ELOA2 |
| GSTA2 | HDHD3 | PPBPP2 | Alkbh3 | TSC22D3 |
| MDP1 | FAM114A1 | CCDC186 | C8orf48 | NFYB |
| EIF5B | C12orf10 | NUSAP1 | MAST1 | GMEB1 |
| DICER1 | Usp32 | ENTPD3 | ADRA2C | SPINK13 |
| TECPR2 | ARHGAP27 | MORN2 | SLC28A2 | RIBC2 |
| ANKRD27 | C20orf144 | TNXB | TMA16 | LY6H |
| RAD51 | SDC2 | ZNF444 | MAFK | GTSE1 |
| KLHDC3 | RWDD2B | EFTUD2 | MAD2L1BP | MAGED4B |
| DEFB123 | EXO5 | CTSD | CCPG1 | MARS2 |
| CBWD5 | AQP4-AS1 | COX6A2 | IGLL1 | QTRT1 |
| ERCC8 | PARS2 | GAST | C16orf71 | XLOC_008362 |
| VCP | GOLT1B | NOL7 | GPER1 | ST6GALNAC3 |
| ANKRD36BP1 | RBX1 | MB21D2 | C8orf58 | DDX6 |
| MRPL3 | MYO1C | MRPL2 | TWF1 | ERBB4 |
| IGF2 | LMNTD1 | DNAJC19 | UBE2D1 | SCARA5 |
| NO_MATCH_181 | SFRP2 | NAV1 | C11orf65 | CHRM3 |
| ZMAT2 | SNHG7 | NO_MATCH_50 | CYB561D1 | AQP4 |
| GRM4 | TBX15 | DLAT | CHCHD3 | BRCC3 |
| RAB43 | CNR1 | FRMD1 | MAGEA4 | RBBP4 |
| SF3A3 | ULBP1 | SDHAF2 | ZFYVE26 | LOC105371493 |
| ZNF621 | LY6D | PEX2 | PTGES3 | TRAT1 |
| CSNK2B | CFHR2 | C20orf85 | PPP1R2P3 | SPANXB1 |
| SHF | FA2H | LOC102725035 | XLOC_l2_006862 | STAP1 |
| PSORS1C1 | KCTD13 | DEFB121 | GALR1 | BLMH |
| OR8G2P | ACYP1 | TEX12 | NXPH4 | CLDN3 |
| CEP41 | MYOG | PRSS22 | TIPRL | CTNNA3 |
| OR8B8 | SLC22A2 | SLC25A31 | VPREB3 | TACR2 |
| SLC35G2 | C22orf23 | TOMM40L | BYSL | ACVR2A |
| MT1E | NO_MATCH_147 | UGT1A10 | UBE2E2 | TTC39C |
| LCE2A | CKS2 | LEP | ABCA11P | GPR55 |
| ANKMY1 | SPRY2 | FGF1 | HCAR1 | CBWD2 |
| MAGT1 | AKAP13 | PDIA3 | PDZD7 | SESN2 |
| PMF1 | CGRRF1 | KRT79 | SSBP3 | P2RY14 |
| NLN | C16orf74 | FOPNL | CORT | IREB2 |
| FBX09 | MATIA | ARHGAP25 | PDCD6 | MAP2K7 |
| ORM2 | SULT4A1 | CLDN4 | NAPRT | NT5DC1 |
| FAHD2A | BTNL3 | PSMB5 | LDLRAD1 | SLC25A23 |
| NABP2 | PI4KAP2 | C1orf189 | CHD2 | GPR87 |
| C12orf45 | EFNB1 | COPS8 | TMEM171 | YARS |
| EBLN2 | GEMIN2 | ARF5 | CEPT1 | TTC12 |
| APBB3 | TLE2 | ABT1 | PPP1R21 | KCNN1 |
| SLC35G5 | OR14C36 | PTCD3 | TAGLN | RBM23 |
| WBP2 | KLHDC2 | Ccdc28a | CHTF8 | HTR6 |
| NAP1L2 | LHPP | MRPL4 | GMPR2 | WIPI1 |
| XLOC_012729 | NT5DC2 | XLOC_l2_009500 | GTF2H2C2 | CRHR2 |
| OR52N4 | ZSCAN22 | DKK4 | GGTLC1 | LINC01567 |
| RRN3 | USP12 | STK4 | GPR25 | NPBWR2 |
| WISP1 | IL15 | ZNF214 | CD27 | TMEM52B |
| MRPL23 | S100A2 | SP8 | PELI1 | FAM111A |
| GRIA2 | CCR5 | FABP4 | MRPS15 | ENPP6 |
| FAM98A | RXRA | ABL1 | TMC6 | ARHGAP11B |
| IL36A | CDC73 | ANAPC10 | ERICH2 | POLR2K |
| UAP1 | BCDIN3D | TMEM17 | HYAL4 | EFHC2 |
| GLIS3-AS1 | TP53BP1 | TBC1D10A | HYPK | ELAVL2 |
| EIF5 | C10orf120 | XLOC_l2_011911 | C11orf45 | PXYLP1 |
| POLD3 | ADGRD2 | ATP6V0E2-AS1 | RNF181 | CCDC170 |
| SLC46A2 | ARL2 | GRIA3 | ATG12 | NO_MATCH_210 |
| TEAD1 | STK17A | MSRA | XLOC_001087 | ETFA |
| RPL7L1 | AKR1C2 | RHOT2 | STXBP4 | LEPROT |
| IL20RB | USP15 | PRDX6 | ANGPTL2 | GPR39 |
| XLOC_l2_009833 | C9 | NO_MATCH_165 | CEACAM8 | USP39 |
| CRYZ | C20orf166-AS1 | IDH3G | SLCO1A2 | DPY19L4 |
| NCK1 | CD82 | TFAP2A | NOL10 | FBXO11 |
| FLAD1 | MUC1 | WDR54 | NAT8B | RASGRF1 |
| TBC1D29 | IGFL3 | ELANE | XCL2 | BTN3A2 |
| CNBP | PLAC8 | C6orf223 | MIA3 | PCGF3 |
| TSPAN32 | PITPNC1 | LRRK2 | HTRA4 | CHRM5 |
| TAS2R5 | SEC22C | Eri3 | C10orf55 | SNN |
| RBSN | MATK | MILR1 | KTI12 | ZNF431 |
| FDPS | TGFB1 | ROR1 | C16orf58 | ALG6 |
| NPDC1 | P2RX6 | RPS27 | EXOC3L2 | MUS81 |
| FBLN2 | CMC2 | RRP7A | SHISA4 | F2RL3 |
| XLOC_012726 | SLC10A3 | CISH | CERK | KCNE5 |
| OR6M1 | TIGD4 | FRMD3 | TTC39B | NO_MATCH_275 |
| ZNF544 | OR6W1P | PNLIPRP2 | NFIC | HDDC3 |
| WDK4 | CCDC51 | ATP5C1 | NAA10 | DDX23 |
| NO_MATCH_225 | DCLRE1B | CD33 | ASGR2 | NDUFAF1 |
| ITGB1BP2 | FAM69A | CHEK2 | CCR6 | BRINP2 |
| NRG1 | C2orf88 | IHAP2 | AGBL4 | TMBIM6 |
| B4GALT7 | NO_MATCH_82 | GPR83 | CLDN12 | KRT24 |
| TMEM199 | SNAPIN | SAFB2 | TSHR | PCDHB3 |
| NME8 | CD207 | TAAR8 | RPGR | SAMD4B |
| NUDT2 | TAB1 | GLRX | CXCL8 | ABCB9 |
| SNX22 | DDIAS | EPHX2 | CCL23 | TMEM252 |
| PRDX3 | BIRC7 | DUPD1 | CRB3 | LRRC14 |
| NME1-NME2 | FAM19A5 | CLC | EPHB1 | TXNL4A |
| ST3GAL5 | EXT2 | HAPLN3 | ALG10 | NGLY1 |
| HSD17B13 | CBX8 | LINC01260 | FAM166B | CPO |
| NOP2 | ZSCAN31 | MSRB1 | PAK3 | SPICE1 |
| CS | SHC1 | Clk3 | KIAA0087 | FAM111B |
| CMAHP | MRPL53 | TLX3 | EXOSC7 | UGT3A2 |
| RRP36 | LMO1 | CHST5 | SPATS2 | ZNF837 |
| TARP | SH3GL1 | MCM5 | RPS20 | NLGN4Y |
| INPP1 | CDK6 | HSBP1 | CHST12 | DNAAF4 |
| TSPAN15 | MYD88 | TCP10L2 | NO_MATCH_174 | MAP2K1 |
| TNFRSF9 | C8B | DIRAS2 | LAPTM4A | TEKT4 |
| Scrn2 | FNTA | SEMA3C | CENPW | FAP |
| SMIM21 | GTF3C4 | SNAPC1 | NDUFA12 | YIPF5 |
| C17orf49 | ADGRL4 | TSPAN33 | HRH2 | PCDHGB1 |
| MAP4K4 | DYNLRB2 | OPN1SW | NDUFA13 | TRMT10C |
| PRPF38B | TMOD3 | CASP3 | UBL5 | PUM3 |
| CALHM2 | HLA-DOA | EFNB3 | TOMM20 | MAX |
| SCRN2 | GABRA5 | HLA-DQB1 | GNL2 | SYPL1 |
| EIF1AY | JRK | PPM1A | WNT4 | TSHZ3 |
| NO_MATCH_109 | NPPB | MAP6 | RSAD2 | SPRY1 |
| ILVBL | DLST | INPP5A | ITPRIPL2 | TRAF5 |
| PEX11G | FAM124B | OIT3 | HIST3H2A | TECTB |
| TROAP | FHL5 | SPZ1 | PSMC5 | NIM1K |
| GGCT | ZC2HC1B | CCDC66 | IL36G | OR10H3 |
| TRAPPC2 | LDHAL6A | C1R | XLOC_001866 | TRPS1 |
| C14orf119 | ARMC12 | NDUFA9 | ST6GALNAC5 | PCNA |
| CDC37L1 | CLPTM1 | PTGS2 | Dolpp1 | ZNF41 |
| SLC35G1 | CCNYL1 | MGARP | MFAP1 | PHEX |
| SYT12 | DTYMK | NO_MATCH_17 | SUN2 | C2orf69 |
| ALKBH8 | TPMT | IQCF1 | XLOC_003758 | ANGPT1 |
| ZBTB8OS | ARSI | C1orf43 | C2orf73 | ZNF354C |
| CHMP5 | CCDC82 | BCAS4 | TBC1D31 | CSNK1E |
| ZNF576 | PLPP4 | STMN4 | C4orf36 | BTG1 |
| ELL | IMPA2 | RSPH14 | EPHA5 | ALCAM |
| ASAP3 | LMBR1 | SERPINB8 | LIN37 | BAG4 |
| PIGN | ZNF141 | ZNF701 | SPATA32 | KANK1 |
| RGL1 | FLVCR2 | RNLS | XLOC_l2_015196 | DCLK2 |
| RMI1 | ADORA3 | APPL1 | TIAM1 | MCCC1 |
| SH3KBP1 | AURKB | ABTB2 | ZDHHC12 | MIER2 |
| SLCO6A1 | CLEC4C | LGI3 | THUMPD3 | IFT122 |
| NUDT13 | ACYP2 | AKAP7 | SCNM1 | RNF10 |
| MCF2L | BUB1 | HECTD3 | GIMAP8 | GFER |
| RIPK4 | BEND7 | DNAH6 | PERM1 | MAPK4 |
| CPSF2 | ORAI2 | PNLDC1 | XCL1 | NCMAP |
| SIPA1L3 | LILRA1 | SLC2A2 | NPIPA5 | DDHD2 |
| NIPA2 | TAF7 | RHBDL2 | NAGPA | ST8SIA5 |
| CUL3 | DIS3L2 | FRMPD4 | BEX2 | CCDC136 |
| BATF2 | XLOC_l2_015133 | GNG13 | GLB1L2 | THOC7 |
| KPNA1 | CDADC1 | NDRG4 | CLVS1 | MYO19 |
| AFF2 | WTAP | TTC9B | HNRNPDL | SPG21 |
| PAFAH1B1 | MRPL48 | CPE | NO_MATCH_263 | PADI4 |
| USP33 | ZMAT3 | GK | KIAA1456 | KLHL23 |
| RBM25 | NRG4 | ATG4A | CPA5 | IQCF3 |
| MORN5 | FLJ37201 | GPX8 | DNAJB8 | CD1E |
| MED24 | SIRPD | HIGD1A | ARF3 | TMEM56 |
| SIGLEC5 | H1FX-AS1 | LILRB5 | RPL36AL | MRPL21 |
| TMEM131L | DDX41 | OR10G2 | MTERF3 | LHCGR |
| SLCO1B3 | CH507-42P11.6 | DACT3 | CXCR4 | DCBLD1 |
| STARD8 | NEU2 | SYNC | CCDC174 | SSTR4 |
| PCDH10 | CLPS | SLC6A14 | PNPT1 | POMT1 |
| MSH5 | NEK8 | FBXW7 | USP8 | KLHL7 |
| COPA | NO_MATCH_257 | PLSCR1 | TOMM6 | RXFP1 |
| ZNF37A | CRTC2 | PPP1R14A | ZDHHC2 | 2-Sep |
| CCL15 | TRUB1 | RANGRF | CHPT1 | SPATA13 |
| DDB1 | CYTH3 | STAU1 | Mbtd1 | DCTN6 |
| GAS6 | INSL3 | XLOC_011321 | AP4B1 | KIR2DL3 |
| ERAP1 | KATNA1 | P3H1 | FGF17 | AKR7L |
| SLC4A2 | PLA2G12A | USP42 | XLOC_013643 | FAM78A |
| SMARCA5 | BTNL9 | NO_MATCH_81 | BORCS6 | ESRP1 |
| FYB1 | PLAU | HMOX1 | BAALC-AS2 | SLC15A1 |
| ELMO1 | KLHL35 | RPP25L | ELK3 | SLFN5 |
| MAGI1 | NGF | PNPLA5 | PFN2 | MCCC2 |
| PALD1 | CC2D1B | RPP30 | SFXN3 | SYNPR |
| OR11H12 | GLRA3 | TTC31 | FAM109B | CYP27A1 |
| PSD2 | CKLF | ALDH1L2 | CUL4B | CUEDC1 |
| PRSS35 | DDX11 | ZDHHC15 | PLA2G1B | ADCYAP1R1 |
| WAC | ABCF2 | EEF1D | PNMT | ART3 |
| DGKB | TRIP11 | KCNQ1 | MAPK6 | IL13RA1 |
| NOP9 | PAPSS1 | RASL11A | PODXL | ZNF660 |
| AQP9 | MRPL33 | DIP2A | B3GALT1 | HNRNPU |
| EFCAB3 | FAM26E | MOGAT3 | SCAND1 | USP44 |
| TMEM161B | ZCCHC7 | ZBTB43 | WRAP53 | RNASE3 |
| VRK1 | GBP2 | C1orf158 | ARHGEF6 | PRIM2 |
| ADAM18 | STAT4 | HCN3 | SNRNP25 | FZD7 |
| C12orf76 | Pkdcc | LIN52 | IFT27 | OPCML |
| CSAG3 | KLK10 | CDPF1 | PLEKHG2 | AUH |
| ACTG1 | NO_MATCH_129 | AEN | FAM135B | SMPD4 |
| LRRC56 | C9orf62 | TRIM44 | DNAAF2 | OR4F16 |
| CAMK1D | YBEY | USP49 | RPUSD3 | MAG |
| CHRNA7 | PPP1R2P9 | ALG1 | NUP35 | TRHR |
| NO_MATCH_166 | UBE2L6 | CCNY | STK26 | PLXNA3 |
| UBE2D2 | TMEM131 | TMX4 | CERS2 | CORO2B |
| SH2B3 | CRIP1 | YEATS4 | KLF6 | RASA3 |
| ZNF436 | MRPS25 | UBE2Q2 | IHEM5 | ACTR1B |
| RPL24 | C8G | ATP5L2 | SLCO3A1 | CCR10 |
| ACOX2 | ZNF596 | KYAT3 | KCNA1 | RAD51B |
| NDUFA11 | TAS2R16 | LRFN5 | CD80 | PIGQ |
| CPLX2 | AGK | SKP1 | NO_MATCH_212 | HCAR2 |
| SP2 | KRTCAP2 | TDO2 | RBM33 | GCGR |
| GJA4 | CHST10 | PYCR2 | ATOH1 | MAP4K2 |
| APOBEC3D | ATP6V0E1 | LY6E | VWCE | MID1 |
| XLOC_l2013192 | TEX29 | GSTP1 | PAM_16 | SRSF9 |
| ZFPM2 | NAXD | MRGPRX2 | KIRREL3-AS3 | FCHO1 |
| HNRNPUL1 | GANAB | SCO1 | SCGB3A1 | IMEM139 |
| FAM120C | CNBD2 | CLMP | AP5M1 | ZNF622 |
| CMTR2 | GRM2 | TMEM176A | ABHD10 | OLFML2B |
| PPME1 | C9orf116 | TMEM213 | ADCYAP1 | NOCT |
| CLVS2 | ZNF30 | XLOC_014105 | CYB561A3 | TYRP1 |
| INPP5K | GPSM1 | LRIG1 | SLC3OA5 | NO_MATCH_78 |
| TMED8 | ZNF718 | NDST1 | GTF3C2 | ACKR2 |
| CYB5D2 | SULT1A1 | MRPS16 | ALG9 | RABEP1 |
| UBXN11 | QRFP | VAMP2 | UBL7 | STRIP1 |
| TXNDC17 | FBLIM1 | ZNF462 | PKLR | TP53RK |
| SPINT1 | SORBS3 | FAM102B | C14orf79 | PPP1R12B |
| ARAP3 | UBE3C | VMAC | ZFYVE16 | SLC39A3 |
| FASN | KRTAP10-3 | TRIM42 | RIN2 | SYNGR2 |
| RIPK1 | INTS14 | PAH | PMF1-BGLAP | VILL |
| SLC30A4 | XLOC_010017 | SLC22A9 | GCSAM | ST7 |
| NEXN | USP3O | Cdk11b | YBX2 | NUP155 |
| CDK15 | TRAM1 | PRAMEF10 | ZNF324 | TRPV5 |
| CXCL9 | SAMD12 | SQLE | C20orf203 | DEFA6 |
| IHAP4 | C1orf64 | SERPINA9 | CTDNEP1 | PCK1 |
| DCXR | SPNS3 | FAM19A2 | RAB42 | KCNK7 |
| IL6R | TRMU | SERPING1 | HAPLN4 | NO_MATCH_71 |
| XLOC_l2_006010 | TSN | NDUFS6 | KIAA1324L | NO_MATCH_91 |
| C17orf62 | NEURL3 | CD300C | TMEM161A | NMT2 |
| GIN1 | AHSA2 | TAS2R3 | ALAS1 | SPAG8 |
| PCLAF | CD68 | UBE2D4 | ARMC5 | EFCAB7 |
| ADGRB1 | RENBP | VN1R3 | DGKE | NCAPG |
| TPRKB | KCNK16 | C16orf46 | SIGLEC12 | C22orf31 |
| AGMO | RPL13AP17 | LINC00260 | GNB1L | GPM6A |
| EIF3E | ING1 | GNAZ | GPR89B | SERTAD3 |
| LINC02347 | TNFSF10 | DNAJB7 | SPDYE11 | SRSF11 |
| RNF180 | ACY3 | INSL5 | FCGR3B | GTSF1L |
| NO_MATCH_18 | RNASE13 | HMGN2 | RALBP1 | FFAR1 |
| NRIP2 | FAM181A | CDK19 | MC2R | RPS16 |
| TSPAN16 | LYZL2 | TUFM | CLDN2 | BDKRB2 |
| ARMC1 | SLC35F6 | TRIM2 | ST8SIA1 | G3BP2 |
| HEXDC | WDK6 | PTH1R | CPSF4 | SUSD6 |
| PTCD1 | ENOX2 | TRIM69 | CIB1 | PHF5A |
| FIBP | MTMR9 | MRPS2 | SRSF3 | SLCO2A1 |
| ZGPAT | PPP1R42 | TRIM49C | LOC390877 | TRIM21 |
| NPY5R | AKR1A1 | TSFM | TRIM73 | SUMO2 |
| PIP5KL1 | OSTC | NO_MATCH_68 | DEFA5 | SSBP4 |
| ARPP19 | ABCC3 | GNAI1 | SALL4 | XLOC_013901 |
| MAK | CRYBB3 | PSAT1 | OR4D1 | CNKSR3 |
| PPP1R15A | KCNAB2 | SLC6A11 | THEG | ABI3BP |
| FAM71D | MAP2K6 | XRRA1 | ZNRD1ASP | FAM131C |
| NO_MATCH_163 | RBBP6 | TRIM9 | NO_MATCH_76 | EIF4EBP1 |
| MAEL | PRH2 | PJA1 | C11orf58 | CDC27 |
| FAM104B | FNDC4 | NCOA5 | B3GAT2 | PPP1R14D |
| SMU1 | CIB4 | VPS53 | EPN1 | GAPT |
| OR6V1 | APOC2 | LYZL4 | TRA2A | TPTE2 |
| NIPBL | UQCRFS1 | TNFSF13B | MCM2 | ZNF350 |
| MGST1 | GNG2 | CABS1 | GLUL | WBP1 |
| FMO3 | C2orf76 | MIS12 | EIF2AK3 | PIAS2 |
| OR8H2 | SRR | KCNK9 | TSPAN6 | GPR137B |
| RNF144B | Ttll5 | IL11 | NO_MATCH_19 | 9-Sep |
| SLC25A5 | PNMA6A | PHKB | ETNPPL | NKRF |
| NPSR1 | SGCD | SHISA2 | TAAR2 | TTLL12 |
| CCDC24 | ZNF706 | SHMT2 | PPID | CORO7 |
| PAFAH1B2 | IMEM218 | NO_MATCH_255 | GPR141 | NO_MATCH_121 |
| BAG2 | MAPK10 | TRAPPC1 | SMAD3 | POLK |
| RASL10B | WWC2-AS2 | PCDHB5 | VIPR1 | SELL |
| CEACAM21 | CCL17 | STC2 | CD200 | Kdr |
| SLC35A5 | DDX39B | CLEC5A | PNMA2 | ZSCAN5A |
| DZIP3 | SPATA7 | ZNF585A | MCRIP2 | ARHGEF10L |
| HMCES | STC1 | TRPV3 | SIK3 | KRT26 |
| UBXN10 | DCTN1 | OR2G6 | FAM173B | C6orf58 |
| SLC22A11 | POFUT1 | DUSP1 | CDH13 | CYP11B1 |
| TTR | CA12 | Cdk13 | GPX4 | CDHR5 |
| PRDM4 | ITGB3 | MRGPRG | RPL27 | PDZD8 |
| H1FNT | SETD4 | TMEM18 | LAMTOR2 | LPAR5 |
| EHBP1 | IMEM80 | PLCL2 | ALS2CR12 | TBL3 |
| METTL16 | CYB5R4 | WISP3 | TMC8 | ZNF513 |
| LIN28A | FAM209B | TOR3A | CFAP46 | EARS2 |
| ZNF829 | RIMBP2 | NINL | EMSY | NO_MATCH_2 |
| XLOC_l2_009328 | CFAP65 | LCN6 | SLC34A2 | GAGE5 |
| PI3 | RASGRP2 | TUBB | TEPSIN | FAXDC2 |
| RUSC1 | ASPHD1 | NO_MATCH_215 | OR2B11 | GRK6 |
| ARC | IL34 | NO_MATCH_269 | PAXX | CD53 |
| PPP1R1C | GCLM | MPHOSPH6 | SLC38A5 | PCDHA10 |
| PRG3 | P2RY10 | NEBL | MICB | SULT2B1 |
| PLA2G3 | TPBG | PFKP | PDHA2 | SPINK2 |
| PRODH | SFTPB | TCAIM | ADAM30 | ALPP |
| LOC401296 | GFOD1 | HS6ST1 | HRASLS5 | ABHD16A |
| WFDC2 | IRF4 | DUSP19 | PPP4R2 | RETNLB |
| XLOC_l2_011027 | LANCL1 | TAAR9 | C12orf57 | KRT83 |
| NO_MATCH_92 | Pin4 | MID1IP1 | XLOC_l2_005718 | LRTM1 |
| FCAR | GLIPR1L1 | THEMIS | Nalcn | PHKA2 |
| NAMPT | C15orf32 | CYP3A7 | PLGLB2 | DUSP14 |
| VN1R4 | OLFML3 | OR5D18 | PCBD2 | HDAC7 |
| NO_MATCH_150 | XLOC_l2_013931 | NO_MATCH_272 | CORO1A | TMPRSS11B |
| MOXD1 | MRPL57 | SERTM1 | SYT11 | SLC39A5 |
| TTC21A | CRADD | NUAK1 | DNAJC3 | SNW1 |
| PPP2R2C | RUFY2 | DUOX1 | NAAA | EPS15L1 |
| CLEC18C | NO_MATCH_192 | KLHDC8A | NICN1 | SPRED2 |
| INS | F2RL1 | ACADVL | TCTEX1D2 | PEX6 |
| OGDH | POLE3 | DHH | COBLL1 | CD1D |
| AP3S2 | TAS2R7 | HENMT1 | SSH2 | ELF5 |
| C19orf47 | RPL14 | ANKRD54 | ZNF774 | GTF2I |
| NO_MATCH_24 | PTRH1 | ACP6 | ZNF607 | OR14J1 |
| OR2AK2 | RAB28 | ZNF485 | PCED1B | MED11 |
| FAM174A | FAM234B | SPANXC | METTL9 | DPF3 |
| NO_MATCH_65 | CYBRD1 | ZNF76 | ZNF667 | RUNX1-IT1 |
| DEFB128 | TPM4 | ZNF671 | LY6G6F | PPP3CA |
| BARX1 | C6orf10 | GPR61 | P2RY11 | THAP7 |
| PRPSAP1 | C3orf20 | HIPK4 | INSIG1 | TBXA2R |
| PCBP4 | GLT1D1 | CLDN6 | CCDC151 | ZNF530 |
| KRTAP10-11 | ADGRG7 | NDUFAF2 | GLTP | TNNI3K |
| HOMEZ | CKS1B | OR10J5 | TTC30B | POLR2D |
| RACGAP1 | SCML4 | KRR1 | PRTN3 | SNX25 |
| PBDC1 | SMTNL2 | DYRK4 | ALG2 | MFSD1 |
| NLGN3 | NAA38 | CWC15 | TAAR3P | ANGPTL1 |
| BAGE2 | NO_MATCH_9 | LIPT1 | KLKB1 | LENG1 |
| ZNF221 | CAMKK2 | CENPS | KRTAP4-2 | BRIP1 |
| Spire1 | NO_MATCH_56 | RPL26L1 | LRRK1 | SMCR8 |
| VAX2 | FAM180A | RIPOR2 | RNGTT | IL7R |
| RAD51AP1 | CCT7 | KLHL34 | MCM7 | HTR2A |
| NMD3 | CNN2 | GPR20 | LBR | SRSF5 |
| HIST1H1T | PHB | POLI | CST11 | SLC25A1 |
| MAP7 | TNNT2 | CBX6 | HNRNPD | PIK3R1 |
| FAM189B | FUT7 | PSD4 | AMPD2 | GADL1 |
| LSMEM1 | FAM198B | MACROD1 | CAPN11 | TEX13A |
| PRR3 | ZNF415 | ICE2 | IFIT5 | USP11 |
| EMID1 | MKNK1 | PLEKHF1 | GSK3B | HP |
| SPARC | AGFG2 | MAIP1 | SFSWAP | FAM78B |
| HPS6 | HNMT | XLOC_004271 | GPS1 | XLOC_l2_006797 |
| PGLYRP1 | PPP2R3C | NO_MATCH_27 | TIMM10B | PRPF31 |
| TRO | BAK1 | HIF1AN | ECE2 | MYPOP |
| GHITM | CDK20 | ENPP1 | GTF2E2 | KCNIP2 |
| PMPCB | B4GALT3 | GLYAT | PODN | SMYD1 |
| ANKRD20A4 | RNF125 | CREBRF | ADRA1D | CACNG2 |
| N4BP2L1 | HSDL1 | DEFB104A | HMGN4 | OPRD1 |
| NO_MATCH_54 | UVSSA | MGAT4B | TULP3 | ZNF720 |
| DPEP2 | DRD3 | PTPRE | RAMP2 | USP4 |
| SDHD | PCGF2 | RXFP3 | AEBP2 | ZNF555 |
| FGA | AUNIP | APBB1IP | PCCA | MRGPRD |
| TTI2 | BLOC1S6 | SPRYD4 | TGDS | DNAJB6 |
| SKAP1 | GIP | CACNG7 | ATL1 | PROKR1 |
| SH3YL1 | TMPO | ALKBH5 | SPNS1 | TFPI |
| OTUD3 | DCTPP1 | AQP1 | TYMSOS | ST6GALNAC2 |
| SAMD7 | NO_MATCH_231 | OSTF1 | MRPS23 | PRSS50 |
| SRBD1 | MLLT11 | ARID3B | UBR7 | TBL1XR1 |
| FLJ13224 | DSCR10 | NO_MATCH_53 | AKIP1 | PASD1 |
| MSTO1 | C8orf44 | BIRC8 | C1QTNF6 | CDKN1A |
| NO_MATCH_214 | UGDH | ADRB1 | SNX3 | RGMA |
| NO_MATCH_131 | Dock10 | GDNF | H2AFZ | PIGB |
| EPSTI1 | RNF135 | UBQLN2 | KRTAP15-1 | TACC1 |
| NMRK2 | PRSS58 | EFHB | ZNF789 | COG7 |
| PTGER3 | CNN3 | ZMYND19 | DPCR1 | CCDC94 |
| LRRC42 | ALG10B | PQBP1 | GSTO1 | SNRNP70 |
| VSNL1 | ICA1 | RHEB | XLOC_014209 | CEACAM7 |
| ADGRA1 | CENPK | PAGE2 | SAP130 | MTNR1A |
| NUF2 | ANAPC16 | KRTAP19-7 | CIRBP-AS1 | YTHDC1 |
| VSX1 | ANKRD53 | CBFB | LINC00115 | USPL1 |
| NUP160 | FAM214B | XLOC_l2_014086 | COPS3 | ZNF518A |
| ZNF26 | TOLLIP | RRS1 | SDCBP | NO_MATCH_73 |
| FADS1 | LRRC18 | PAQR3 | INO80E | SLC52A1 |
| TNNC1 | LRIF1 | KDF1 | HPSE2 | TPCN1 |
| Ola1 | HEPACAM2 | RHOJ | ACTC1 | MAP4 |
| PISD | TTLL6 | SENP6 | NO_MATCH_37 | CYSLTR1 |
| TESK2 | OLFML2A | TIA1 | FAIM2 | PCM1 |
| WBP2NL | EIF2AK4 | GPR152 | TSPO2 | CA14 |
| XLOC_l2_004844 | NUS1 | KRTAP3-3 | TFCP2 | FBXO22 |
| C12orf49 | SFRP1 | CCNL1 | ICMT | SARS2 |
| SMIM12 | LGALS1 | RPF2 | VAMP4 | KANSL1L |
| ASAH1 | RNF219 | UGT1A4 | CRHBP | LIPC |
| PDE1C | M0RN4 | TPRG1L | ZNF223 | SREK1IP1 |
| XLOC_007477 | TXNL1 | OMP | NO_MATCH_122 | KIAA1161 |
| FAM126B | SLC26A2 | ELOVL2 | DHODH | GGT7 |
| UBE2T | S100A3 | ARPC4 | DHRS7 | ARSK |
| CNOT11 | MRAS | NUDT22 | POU2F2 | ALPI |
| RAB27A | MARK4 | TIMM17A | SGO2 | IRF8 |
| MDK | IL9 | WDR55 | ANPEP | CSDC2 |
| CDK4 | GPR32 | DDAH2 | IMMP2L | PRM2 |
| ARL6IP4 | SLC25A41 | ATP1B4 | NDUFB1 | PNMA3 |
| WRNIP1 | DHRSX | CXorf56 | ARHGEF25 | SPDYA |
| HLA-DPB1 | SRRD | GNAO1 | NO_MATCH_189 | NO_MATCH_110 |
| SSSCA1 | SYNPO2L | UQCR11 | LDOC1 | DHX32 |
| ATF7 | ESD | ICOSLG | MCM9 | PTPRH |
| CCDC162P | OSGEP | DPY30 | C5orf22 | MGEA5 |
| PYROXD1 | DKK3 | SULT1C2 | CDAN1 | ZNF496 |
| AK6 | GNPTAB | BTBD8 | CLP1 | ZNF281 |
| IL12A | CCSAP | TDGF1 | GZMB | XLOC_l2_008259 |
| NR1D2 | XLOCO_10072 | IRGC | ACAD9 | ZNF136 |
| ERP29 | L3HYPDH | HAUS6 | BUB3 | ATF7IP2 |
| TIMM50 | OBSL1 | MXD1 | IFI6 | PLOD3 |
| RPS6KA4 | HSF2 | TOMM34 | VWA3B | AMOT |
| LOC554223 | NO_MATCH_191 | ACKR1 | ADGRG6 | ZNF260 |
| HESX1 | NAP1L5 | PAK1IP1 | LSR | ILKAP |
| THRSP | TALDO1 | KLK4 | LASP1 | MTO1 |
| FGF6 | ZNF384 | RET | IGSF10 | MORC2 |
| CTXN1 | NO_MATCH_57 | TSTD2 | KRTAP10-1 | HLA-F |
| CYP3A43 | PRAC1 | VPS25 | KCTD4 | PLEKHG6 |
| LYZ | TCTA | GATM | GRPR | ZGRF1 |
| GBA | PTS | TMLHE | ARRDC3 | NR3C2 |
| SPRTN | SELENOT | PCAT4 | CCDC60 | SNX14 |
| PIGC | EIF5A2 | DOCK8 | NMB | FTSJ3 |
| NO_MATCH_161 | C9orf139 | LMNA | C5orf46 | NO_MATCH_12 |
| EEF2KMT | OR51E1 | HTR3B | STAG3 | MTNRIB |
| ECI2 | TRIB3 | MEI1 | XLOC_013689 | THAP12 |
| VASN | IL36B | ZNF775 | TIMP2 | PLEKHG5 |
| TAS2R60 | KIRREL2 | CDK16 | PCLO | TUBD1 |
| SLAMF8 | SACM1L | ZMAT4 | HLA-DPA1 | SRSF4 |
| HIST1H4A | LINC01559 | FBXL17 | IPO11 | B4GALT6 |
| VDAC2 | MORN3 | SOSTDC1 | CFLAR | C5orf24 |
| OR2W1 | ZDHHC9 | STPG3 | AGBL3 | ASGR1 |
| HNRNPCL1 | WASF3 | COX7A2 | SPPL3 | PKNOX1 |
| GTF2A1L | MAP3K11 | OR2B3 | NRAS | CUL5 |
| LCMT1 | TSPO | CCKBR | SIGLEC10 | CTC1 |
| SNCG | XLOC_007690 | SLAMF1 | CFAP45 | ZNF268 |
| FUT8 | PMP2 | HSPB9 | NO_MATCH_193 | BST1 |
| PRNP | SSX4 | OIP5 | KLHDC1 | CCDC38 |
| UNKL | CHAD | TAS2R40 | PRICKLE4 | TULP2 |
| FAM53B | PUS7L | PLK1 | PLA2G12B | NEUROD1 |
| BTF3L4 | CHRNA9 | Xkr6 | LGALSL | KLHL10 |
| PITPNA | NO_MATCH_117 | TMEM182 | CCDC34 | ITK |
| IMMT | OR1J1 | FAM49B | EZH2 | NXF2 |
| RPS11 | CD99L2 | TBATA | C11orf63 | DUSP28 |
| KLHL29 | DDX51 | FAM71C | BCL10 | MLNR |
| RTN4 | TRMT12 | EIF1AX | TAS2R39 | LCOR |
| TRIM15 | SMLR1 | KRTAP5-9 | KRT1 | CDCA3 |
| CFAP74 | NAA60 | HAUS8 | OR3A1 | GPR101 |
| PTGIR | POLE | UBE3D | LINC00602 | P2RY2 |
| PTK6 | AP1S3 | BROX | NO_MATCH_130 | SLC2A11 |
| DCTN3 | CAMKV | RPTOR | ZNF286A | TMPRSS11A |
| PSMB2 | ZFP64 | SGPL1 | C3AR1 | USP3 |
| BAALC | TRIM50 | TRIP4 | OPN1MW | OR10A2 |
| IDO1 | TREM1 | HIST1H3D | UNC45A | TEX43 |
| FKBP3 | PDHB | Tex261 | RCL1 | EXOC1 |
| TLR3 | BTD | DHRS12 | P2RY13 | FAM50B |
| IFRD2 | ELSPBP1 | USP36 | FGF19 | KLHL36 |
| CASK | CENPP | TRAM2 | LETM2 | ALK |
| TRIM55 | COPS5 | PTPRS | SMAGP | HEYL |
| HTR3A | C9orf163 | MFSD10 | RASL10A | RBMS2 |
| LOC149950 | LINC01554 | FAM193B | PRKAA2 | C21orf59 |
| CCNA2 | TMEM136 | RFX6 | RPS28 | ZNF16 |
| TUBA3FP | SKP2 | GDF3 | MGAT3 | CRHR1 |
| CLEC4M | CCDC83 | COQ8B | PACSIN1 | EPHA1 |
| ANXA13 | BEX3 | SRM | SCYL1 | HLCS |
| DAP | GEM | CLEC2B | FADS3 | OLA1 |
| AK4 | CMPK1 | MAP1LC3B | GUCD1 | GUCY1A3 |
| NRN1 | RAB6B | COX7B | ZNF124 | ASPM |
| IKZF1 | SPC25 | NRM | ZNHIT3 | NRROS |
| TAC3 | MT3 | LOC148413 | EXOSC5 | NTSR2 |
| INHBE | SLC9A1 | FPR1 | C4BPB | SLC29A2 |
| CGNL1 | ATP6V1G3 | TMEM176B | XLOC_013551 | RBM14 |
| GUCA1C | HCRTR1 | TSPAN11 | TRPM8 | CD55 |
| EXD2 | PRR5 | IL1RAP | WBP11 | TEX11 |
| ERGIC3 | STAG3L2 | HHIPL2 | PLXNB2 | TRAF6 |
| NO_MATCH_233 | TRAPPC2L | NECAP2 | PARN | VNN2 |
| HPGDS | PTGES2-AS1 | XLOC_l2_005179 | TMEM50B | LCE4A |
| PAGR1 | NMRAL1 | ARFGAP1 | LINC01366 | ENC1 |
| BMPR1A | ZNF446 | CYP2E1 | GRK3 | CD86 |
| LCN8 | MAPK8IP1 | ORMDL2 | AFM | PTPRN2 |
| NPFFR1 | MICU2 | FGF21 | LYRM7 | ZNF385C |
| TCEA1 | PWP1 | AKIRIN2 | TDRKH | GPN1 |
| FRK | ZNF75A | P4HA2 | MALSU1 | SRPX2 |
| SEMA3E | S100A13 | TSPAN17 | RCVRN | OR4X2 |
| DZANK1 | IZUMO4 | OAZ1 | DEFA1 | SOCS3 |
| DUS1L | CRACR2A | ZG16 | LRRC4 | MARK3 |
| SLC35B3 | NO_MATCH_29 | FGFR1OP | ZDHHC7 | THEMIS2 |
| XK | GNA12 | MESP2 | SERP2 | MRPL41 |
| CXCL13 | RNF14 | IHAP8 | NO_MATCH_176 | GPR182 |
| UNC119 | LRRC52 | TBC1D14 | RPS14 | VSIG1 |
| OR8B12 | TSPAN3 | SCD5 | LYPLA1 | GPR17 |
| C17orf64 | ENTHD1 | PTBP2 | DAB2IP | ZNF326 |
| WDR18 | RMND5A | FCN1 | POLR2A | ADGRE3 |
| ZMYM6 | NTPCR | PSG3 | TCN1 | GPR139 |
| CCL4 | RIC3 | STMN1 | NCR3LG1 | VAC14 |
| MAP3K12 | CD1B | TMIGD1 | COQ6 | NO_MATCH_16 |
| FLJ25758 | COMMD4 | STK17B | HSD17B14 | CLMN |
| NO_MATCH_116 | CSRP3 | WEE2-AS1 | 9-Mar | RBM15 |
| FBXO6 | MTX2 | RAB11A | WDR25 | KLHL20 |
| GALNT10 | TIAF1 | ELP5 | SZRD1 | FFAR2 |
| KCNIP4 | WFDC9 | POMGNT1 | POLR3D | MAOA |
| MAP1LC3C | XLOC_l2_013393 | CDC42EP2 | NSDHL | ERVV-1 |
| APEX2 | IFITM1 | Hkdc1 | MCMDC2 | ALDH1A1 |
| HIST3H2BB | BRINP1 | MC5R | C1orf123 | FPR2 |
| TNFSF4 | USP32 | ZFHX3 | RPS21 | P2RY12 |
| ECEL1P2 | ATF1 | SEMA7A | FBXL15 | BICD2 |
| SELENOW | CLCN5 | CMKLR1 | SLC6A3 | BRD8 |
| LOC613266 | SMAD2 | EXTL3 | CALML4 | HIST2H2AB |
| ATAT1 | CA4 | TLR5 | CACNG1 | DEFB119 |
| TMCC2 | BEND5 | KLHL4 | H1FOO | ATG7 |
| CFAP161 | RAB40B | PCYOX1L | PPM1D | SLC1A5 |
| GAL | CETN1 | OVCA2 | FEZF2 | EIF4EBP3 |
| AKAP5 | TAF5L | STK11 | EVA1B | NO_MATCH_113 |
| PRH1 | USP27X-AS1 | AMIGO3 | LY9 | SSTR3 |
| PFN3 | APIP | CXCR6 | CXCR2 | NEUROG1 |
| APOM | APOOL | CHAF1B | SCRG1 | ZNF296 |
| TEKT4P2 | STK10 | U2AF2 | NECTIN2 | MYL2 |
| TMEM183A | F8 | TMEM31 | HSPA1B | ALPK1 |
| UBD | AGR3 | LYPD1 | AOC3 | TSGA10 |
| FAM216B | NO_MATCH_22 | C19orf73 | ZNF626 | RYK |
| MMD | HDGF | SMYD2 | LINC00982 | ZNF92 |
| EMC7 | RFPL2 | NDUFAB1 | RPS24 | LOC102724023 |
| BCL2L15 | RFESD | SLC35C1 | ITIH5 | OR51M1 |
| RPH3AL | C1orf74 | NPTN | SLC35F5 | IL2RB |
| TOM1L2 | GPR84 | NUFIP1 | CNPY3 | ENPEP |
| STRAP | RNF115 | DHFR2 | DDB2 | XPO6 |
| TMSB4X | NRF1 | RGS1 | AAGAB | EIF3C |
| KDM4B | SPAG11B | MCTS1 | PTEN | RTN4RL1 |
| VHL | RALA | APLNR | CHD1L | VNN1 |
| DCD | LOC100653049 | SBSN | HBS1L | CD300LB |
| STRADA | RFK | LRRFIP1 | G0S2 | EDNRA |
| IHAP10 | TIGD6 | PHACTR2 | NO_MATCH_26 | PIK3R3 |
| MOB3C | KYAT1 | CPA3 | LDLR | PXDN |
| SERPINA1 | FKBP9 | SPA17 | SAMD10 | RBFA |
| TMEM42 | ZNF416 | SMARCB1 | TRIM24 | YTHDF1 |
| PROSER3 | SRD5A1 | ACVR2B | SLC25A37 | CD8A |
| DFNA5 | FOXD4L6 | PTGES2 | SLC25A10 | RNF6 |
| NUDT15 | PQLC2 | RIN1 | GPC5 | Laptm4b |
| XLOC_013119 | CUL1 | DMKN | MLPH | NO_MATCH_217 |
| UBE2G1 | PAFAH1B3 | MTX1 | LY96 | GPR68 |
| PRAMEF5 | ALDH8A1 | MYL1 | YOD1 | NO_MATCH_173 |
| SCAPER | TLE1 | FASTK | PRSS21 | S1PR1 |
| MTFP1 | MOGAT2 | ZC3H3 | CLCN2 | SLC33A1 |
| LEFTY2 | TP53AIP1 | ARMC6 | PDSS1 | SLC4A3 |
| TRMT61B | SRPX | SMR3B | ABLIM2 | CALCR |
| CNTF | PSMD3 | OR2T4 | NPHP3 | CASQ2 |
| TPH2 | BNIP1 | APLN | CCDC28B | EMC1 |
| ID3 | GGT2 | PCDHB11 | CFAP43 | ENKD1 |
| CDCA5 | ARHGAP29 | SNTG2 | PRSS37 | SPATA18 |
| FANCI | PCDHGC5 | CAT | DAZ4 | SUMO3 |
| NFE2L3 | C20orf197 | C3orf52 | C3orf18 | SLC7A13 |
| ADI1 | ZNF252P-AS1 | KLF12 | KRTDAP | MMP12 |
| TRAF3IP3 | MIF4GD | GHRHR | NO_MATCH_211 | S1PR2 |
| DDX20 | TSEN15 | BMP15 | TCP10L | SSTR2 |
| DLGAP5 | KCNS3 | ACSBG2 | ZNF786 | EVI2B |
| PPP1R8 | LHFPL4 | KLHL9 | ADORA1 | FAM83F |
| PPP3CB | CCDC84 | SH2B1 | ZNF274 | PHACTR4 |
| ESCO1 | FXR2 | CLEC4A | SARNP | AFAP1L1 |
| NEU1 | DCAF12L1 | TMEM150A | CHRM1 | GPR78 |
| CSH1 | RASGEF1B | DCAF15 | RPUSD4 | S1PR3 |
| NR0B2 | CENPC | GDF5OS | CCDC40 | SERTAD1 |
| STX3 | IKBIP | LRRC10 | ABCB8 | USP53 |
| SNRK | PSME2 | COL20A1 | SOX12 | PTGER1 |
| ZNF521 | LSM5 | GM2A | GGT5 | ZDHHC5 |
| MAPK1 | EIF4EBP2 | NIPAL3 | MAGEA1 | FAM32A |
| MBTPS2 | CYSTM1 | CCR4 | COX11 | ZNF567 |
| BCKDHB | ZNF580 | ZNF410 | PLS3 | MINDY2 |
| C17orf105 | SPATA45 | FMO5 | C2orf27B | SART3 |
| PHC1 | ARL2BP | NO_MATCH_180 | ZNF436-AS1 | MAS1 |
| RASSF4 | C6orf136 | ANLN | DNAJC2 | ZNF645 |
| AK2 | LZTFL1 | TMEM25 | WARS | CCL28 |
| TNFSF12 | COX10 | LINC01711 | P2RX2 | GPR35 |
| MRPL44 | CXCL10 | EMCN | ECT2L | NFATC1 |
| PLPP7 | TMEM71 | GNPDA2 | LEAP2 | PHF20L1 |
| COL8A1 | SETD7 | TMEM19 | RIOX2 | ABCD3 |
| ANKRD18A | XLOC_l2_008285 | ATOX1 | ARHGEF16 | CD24 |
| TASP1 | C14orf105 | GP6 | MAML3 | P2RY6 |
| GGH | TEN1 | POPDC2 | ZNF672 | ELOA3B |
| NOTCH2NL | PGK2 | LOC102724229 | RAP2C | DPEP1 |
| TMEM91 | STOX1 | GALNT3 | CDK3 | NPR1 |
| ASPDH | TPX2 | GCKR | BLK | RASA1 |
| MED7 | SAMM50 | SKIV2L2 | GLOD4 | SNRNP27 |
| TMEM200B | MTRF1 | TAS1R3 | PHYHIP | LINC00477 |
| HIST1H4G | NO_MATCH_204 | WRAP73 | PPFIBP1 | RTN4R |
| HSD17B6 | ZNF768 | SLC29A4 | FOXP2 | FPR3 |
| CMAS | DDX28 | KRTAP4-12 | PTPRM | LRRTM1 |
| CCL14 | HINFP | RSPH3 | OTC | CLCA4 |
| ZNF473 | XLOC_l2_001972 | NDFIP1 | UNC93A | IL10RB |
| HM13 | LRRIQ3 | POLR2I | STK19 | DPF2 |
| FAM_170_A | PRKCB | NMT1 | STAG3L4 | ZNF781 |
| GNAT2 | TRIM60 | CNOT4 | SKIL | HTR1A |
| STARD5 | SLITRK3 | MAP3K5 | PRKAR2A | PLA2G7 |
| ATP6V1G2 | GALM | HAS1 | GNL3 | VPS54 |
| FHL2 | SATB2-AS1 | FAM71F2 | RAB5A | SCFD2 |
| P2RX5 | KLK8 | KEL | ZSWIM2 | CXXC4 |
| MED20 | TMEM64 | THADA | COQ10B | NO_MATCH_141 |
| ZPLD1 | CSHL1 | USP7 | TRIP10 | KCNG3 |
| LRRC37A5P | PDAP1 | BMP3 | FTH1 | GNB3 |
| GC | PTPN2 | SLC25A13 | SNAP47 | FAM83B |
| CHAC2 | RSU1 | POMC | TYROBP | SMARCD1 |
| UHMK1 | STK40 | RPS17 | UTP14A | LTB4R |
| MRM2 | EEF1A2 | ZNF100 | PRKD3 | CDK11B |
| CABP7 | CCL13 | OR7A17 | KRTAP10-5 | GYPA |
| COPE | GSDMC | NO_MATCH_277 | MYF6 | OR1L3 |
| LINC01587 | C9orf72 | VWA5A | PDCD1LG2 | CIDEA |
| FAM206A | RTL10 | PPIG | DET1 | TRIP13 |
| FAM226A | CYP4X1 | OR8G5 | CEP63 | GRK5 |
| OR8D2 | NO_MATCH_172 | POP4 | OR10AG1 | VANGL1 |
| PRRT1 | CRCT1 | CA5B | SHD | ZNF574 |
| EXD3 | KRT222 | TTC5 | APOA2 | SLC35F2 |
| SMNDC1 | VEGFA | SLC32A1 | GPR174 | C7 |
| TUBB6 | LYPLAL1 | ABI2 | FAAP20 | VPS26B |
| GSTA4 | TRIM35 | LAMA4 | NO_MATCH_168 | SSBP2 |
| ANKHD1 | MT1M | CYB561D2 | CPNE1 | PCDHA9 |
| RSPH10B | LOC400710 | LSM4 | NADK2 | IL12RB1 |
| OR8I2 | AMZ2 | ZC3H14 | SLC29A1 | CD59 |
| SERPINB1 | PRG2 | XLOC_l2_015937 | ADGRE2 | MRGPRX4 |
| ARHGAP15 | CDK10 | GTF2B | LTA4H | TBCEL |
| TYK2 | CHCHD6 | SLC43A1 | PDCD2 | WDR60 |
| CCNG1 | TMUB2 | GAS8 | AIPL1 | NO_MATCH_207 |
| SNX6 | OTUB2 | ARMC9 | KLK14 | CSNK1G2 |
| DLG1 | TEAD3 | EIF3K | PDLIM7 | ATP6V1B1 |
| SSNA1 | XLOC_001272 | KCNK13 | C9orf40 | ZSCAN12 |
| GORASP1 | LIF | HMGB2 | HOXC10 | ZNF554 |
| ERBB2 | TBRG4 | UCP1 | CHIA | ZNF433 |
| RBM48 | AKR1B10 | CACNA2D3 | ARHGAP22 | APBA2 |
| PRR16 | TEF | HIST1H4E | FLYWCH2 | SLC38A4 |
| POLR2G | GLYCTK | PRKD2 | SPATA12 | ASIP |
| NO_MATCH_40 | TOB2P1 | HK1 | IER2 | ZNF615 |
| CAV1 | PTP4A2 | PDLIM1 | FBXO46 | CDHR3 |
| Vps16 | ARHGEF9 | SMOX | RAB15 | RBM6 |
| GMPPA | USP25 | OR8H1 | ARHGAP11A | CNGA3 |
| PSMD14 | ACTB | KIF1B | GRHL3 | USP5 |
| MPZL2 | TMEM237 | BHMT | CEP57L1 | DMD |
| NDUFA2 | RPS6KB2 | FAM160B2 | SLC22A18 | TM7SF3 |
| RNASE11 | THG1L | PLXNA4 | FBXO30 | PYGL |
| EIF2B2 | KIF19 | SLC19A3 | GSTT2B | TRMT1 |
| PTGFR | RASSF2 | NO_MATCH_236 | PUS7 | CNGA4 |
| GKN1 | ADAM32 | HOOK3 | XLOC_l2_015194 | GPBAR1 |
| KCNV2 | PES1 | METTL22 | LLPH | SOCS5 |
| PHLDA2 | DAPK1 | JPH3 | CA2 | TMEM168 |
| TRAF3IP2 | SMIM8 | FDCSP | TBCA | MIP |
| WDR49 | STARD3NL | SLC18A1 | NT5E | DTNA |
| KLHL33 | GPN2 | SPIRE2 | SLC22A7 | FAM45A |
| TCN2 | MZT2B | INPP5D | GOT2 | LPAR1 |
| SP3 | PIH1D1 | C17orf82 | SHKBP1 | LPAR2 |
| NO_MATCH_271 | EPS15 | ELAC1 | CNN1 | ITGA5 |
| GNAL | ADAP2 | ZNF543 | CCDC14 | CUL2 |
| TTC7A | SLC17A4 | PSMG3 | HARBI1 | TTC14 |
| PAPOLA | IMPG1 | SLX1B | TRIM5 | CCHCR1 |
| CELA1 | LINC00898 | RNF113A | KAT5 | ACLY |
| LYRM2 | TBRG1 | VTN | XLOC_009142 | NO_MATCH_35 |
| GJB4 | PARL | NO_MATCH_200 | IKBKG | SLC16A14 |
| UQCRH | BRICD5 | CALR | FMNL1 | 8-Mar |
| AURKAIP1 | SMAD4 | FOXR2 | AIRE | PLXNA2 |
| POGLUT1 | RIMKLB | KIR2DS2 | ZNF200 | ZNF599 |
| LOC107987587 | PEX3 | APOD | TNP2 | PRPF40B |
| PCDHB15 | IL17A | NCOA3 | E2F7 | ENPP7 |
| SFRP4 | PDCD5 | FOXI1 | CMTM4 | ITGAE |
| ASB3 | SRSF2 | KIF2A | SAR1B | UGT2B7 |
| FAM89B | BGLAP | MRM1 | PLEKHG7 | ARHGEF3 |
| TAF9B | SF3B6 | MEX3D | TMEM187 | IMPDH1 |
| GGPS1 | LINC00482 | UBL3 | ARHGEF10 | WDR27 |
| FDXR | CDK5 | DNAJC15 | LINC01949 | CD93 |
| TANG06 | MAGIX | PAMR1 | ZNF24 | BTAF1 |
| POLR3F | XLOC_l2_008203 | ZSCAN2 | PSMA6 | GPA33 |
| RUBCNL | DCAF10 | MCOLN3 | SLC2A6 | UBL4A |
| MAB21L1 | XLOC_l2_009464 | XIRP2 | PSG5 | HTR2C |
| C19orf18 | SLAIN2 | PDS5B | CALN1 | SELPLG |
| RGS17 | DDX55 | CALY | NO_MATCH_93 | NEUROG3 |
| PCNP | XLOC_l2_004129 | CETN2 | HAUS3 | ZNF10 |
| Ergic2 | HIST1H4B | SPSB3 | TUBB3 | KIAA0319L |
| TREML4 | MAP3K6 | ZBTB37 | ZNF385B | SIDT2 |
| GGACT | C1QTNF2 | EPHX4 | LIN28B | GPR75 |
| LACC1 | BCAT1 | CDK2AP2 | VIPR2 | PRKCG |
| RTP4 | DCDC2B | GBA3 | GRM3 | AMBRA1 |
| PRELID3A | FBXL21 | TGFA | LINC00508 | ALPL |
| ATM | CDH19 | ADIPOR2 | LOC652276 | RAPGEF6 |
| NCOR1P1 | TTC16 | DTWD1 | SERPINA6 | GPR4 |
| PCGF6 | KLRC4 | COQ9 | ZCCHC11 | CREB1 |
| TCEAL4 | BEX1 | KLHL3 | PHTF2 | PDE7A |
| TMEM51-AS1 | RBBP7 | NEDD1 | PRPF39 | ZNF420 |
| HCCS | CD8B | SIRT7 | RABL2B | DNA2 |
| CPVL | FAM71B | SNX20 | AMT | USP1 |
| RGR | RAB7B | WDR5B | PTMS | ZNF804B |
| MDH2 | XLOC_003546 | XLOC_l2_008131 | C1QTNF4 | PLEKHO1 |
| HTR5A | CTRL | LINC01553 | TMPRSS11E | VAV1 |
| DHDDS | ZNF792 | ARL17A | GHSR | LYPD3 |
| ISCA2 | ZNF57 | COMMD3 | DPY19L3 | MED31 |
| P2RX1 | BCHE | GSTCD | ZNF467 | NTSR1 |
| NO_MATCH_154 | RPE | SLC2A1 | SPIB | TMEM154 |
| IL22RA2 | THTPA | LINC00174 | NO_MATCH_135 | SEL1L3 |
| MAL | ZNF417 | AIG1 | XKR8 | NGEF |
| SEM1 | COL18A1-AS1 | C4orf19 | LYPD2 | MPP4 |
| ANG | LOC100653061 | ZNF689 | PI4KB | LPAR4 |
| RTKN2 | KIAA0753 | FBXL4 | LTB4R2 | PIK3C2G |
| NDUFA8 | PRR19 | XLOC_003385 | TXNDC16 | COBL |
| COPS7A | SPART | RPL38 | LGALS13 | PVR |
| KLHL32 | TRIML1 | PTH2 | ABCA8 | PLD1 |
| PDHA1 | SLC27A6 | ACSM1 | PHIP | EXT1 |
| Emilin1 | TMEM268 | COA3 | C5orf34 | ADRA1B |
| CDC7 | NMNAT2 | KLK5 | TUBB4B | LSS |
| FAM107A | CCL24 | CRYAB | ARID3A | TRPC4AP |
| MT1H | LYG2 | NO_MATCH_38 | HIST1H2AK | INTS7 |
| TPD52 | S100A16 | PRR20A | ERICH6 | S1PR5 |
| MRPL43 | ZNF843 | PLAC8L1 | TMEM144 | GDAP2 |
| MTIF3 | MRPS26 | HPF1 | PTPN6 | OMD |
| LOXL3 | COL5A1 | CYP2F1 | MEF2A | ENTPD1 |
| MIR1-1HG | CLOCK | XLOC_l2_006425 | ALX1 | ZNF131 |
| SMIM2 | NUTM2F | SCAND2P | PREB | AZIN2 |
| GOLGA6L9 | KLF3 | RXRB | TAF7L | TMEM120B |
| NUPL2 | PPP1R14C | CCBE1 | C15orf53 | ZNF227 |
| OAT | BBS7 | GZMH | TSPYL1 | FANCB |
| DAPL1 | BCL2 | SLC41A2 | MAGEB2 | IL10RA |
| GNAI2 | SERPINE1 | Ttyh1 | NFIL3 | P2RY1 |
| SLC22A14 | FAM122C | XLOC_l2_005687 | ZNF248 | IL21R |
| SMAD1 | NAT1 | ALDH2 | C15orf41 | UTP14C |
| RSPH9 | IGHMBP2 | ZNF578 | OR4D10 | RNF220 |
| NAA20 | PPOX | SPATA6L | HIST1H2BK | SLC22A16 |
| HACD4 | SAP18 | LMOD1 | PSMD12 | B3GNT2 |
| LUM | CAPN6 | TNFRSF6B | ERMAP | ENTPD4 |
| TCEAL3 | EEFSEC | PDRG1 | SMYD3 | OMG |
| PDE6B | PTGES | ADA2 | GSTT1 | MCAM |
| SSX2 | SPRR2A | GOLGA8B | SDC1 | SLC40A1 |
| FYN | DPH1 | IFITM3 | MARS | DHX36 |
| SERPINB5 | CCDC189 | ADAM21 | PUF60 | NFATC3 |
| TMEM169 | B4GALT1 | OR2A12 | NRCAM | ICAM3 |
| RAB37 | GATA1 | WDK5 | FBXO38 | FAM133A |
| ZNRD1 | SNX7 | CIRBP | EWSR1 | PKP2 |
| CDIPT-AS1 | ALDH3A2 | Mon1a | FBXL12 | ADAMTS18 |
| CAMK2N2 | ANAPC11 | ELP6 | PLP1 | BRS3 |
| AP5B1 | SLC35F1 | CLRN1 | LNX2 | HSD17B4 |
| CCDC113 | C1orf105 | LOC107984065 | MEIOC | SPN |
| LURAP1 | MUSTN1 | RABIF | GPC6 | PCDHGB2 |
| C5orf38 | EPPIN | FAM153A | COG6 | MYCL |
| TNFAIP8L3 | OR2A4 | IGSF8 | BLID | |
| XAGE2 | ZKSCAN4 | DMPK | CLEC17A | |
| G6PC2 | S100P | JAM3 | TRPC5 | |
| DUOXA2 | CBWD1 | TSACC | GSKIP | |
| MIR31HG | TNFAIP8L2 | TGS1 | RSBN1 | |
| RTL8B | RPL23 | GAL3ST3 | OR7A5 | |
| UCMA | TSKS | AMHR2 | NUAK2 | |
| CALCA | WASHCI | PLPP1 | PCDHB7 | |
| DCAF12 | LCN2 | ZNF211 | ZNF776 | |
| SDF2 | MFSD8 | SLC25A44 | ZNF670 | |
| RAB3C | SMCO4 | PLA2G15 | LARP1 | |
| ANKRD44 | DACT2 | CDKL1 | GDPD2 | |
| NGRN | IZUM02 | CAP2 | ACER2 | |
| METTL18 | PLA2G10 | TMEM184C | ADGRG1 | |
| CD300LF | ZAR1L | APOC4 | GJB7 | |
| ROPN1 | CCDC69 | TLR1 | VWA2 | |
| BRF2 | ACSF3 | NEK11 | RPL22 | |
| ALG1L | MYO1D | PLPPR2 | AHSG | |
| BTN3A3 | C3orf35 | ARL4D | CCDC89 | |
| GAGE7 | UPK1A | RBP5 | HSPB11 | |
| LOC107987235 | COMT | FGD2 | TAF12 | |
| HNRNPC | GPCPD1 | NO_MATCH_99 | TP73-AS1 | |
| CYP2A6 | BIN3 | RHOF | PRKACG | |
| B4GALNT2 | VCY | FAM221B | CDC14A | |
| APOCI | EMC9 | NOB1 | KRT7 | |
| BPIFB3 | ZBTB32 | PGM2L1 | ZNF692 | |
| XLOC_l2_006131 | HDHD2 | CD151 | LOC102724334 | |
| SUN5 | FAM213B | NT5C3A | RTCB | |
| PTPN23 | SERPINF1 | KRTAP9-6 | CLPSL1 | |
| ZNF484 | EGLN3 | XLOC_l2_014048 | NHLH2 | |
| SELENOI | GNG8 | NDRG1 | SEMG2 | |
| WNT10B | SLC25A6 | WDYHVI | ASB7 | |
| LYSMD1 | NO_MATCH_115 | NPY2R | FFAR3 | |
| APOBEC3G | SSR4P1 | HBP1 | DHRS9 | |
| DEFB129 | CIDEB | SCP2D1 | GABPB2 | |
| PLVAP | ZNF280C | SLC16A4 | C8orf86 | |
| WEE1 | ACTL8 | ERCC1 | LONRF3 | |
| SPANXA1 | SLC10A7 | DUSP3 | PDCD1 | |
| COX4I1 | PTH | CYP17A1 | SLC14A2 | |
| WASHC3 | NR1I3 | SLIRP | PCDHA2 | |
| STK16 | NRBP1 | MSLN | NBPF8 | |
| CDK5RAP3 | PLA2G5 | DNAJB12 | SLC37A4 | |
| GPAT4 | ARHGAP44 | CELF3 | LRRC36 | |
| CDA | SERPIND1 | CXCR5 | CARD8 | |

### EXAMPLES

### Example 1: Materials and Methods for Examples 2-9

a. Generation of Tumor Cell Lines

Tumor samples were obtained from either patient biopsy or patient-derived xenograft. The tissue was minced manually, suspended in a solution of 2 mg/ml collagenase I (Sigma Aldrich, St. Louis, Mo.), 2 mg/ml hyaluronidase (Sigma Aldrich) and 25 g/ml DNase I (Roche Life Sciences, Branford, Conn.), transferred to a 15 mL conical tube, and incubated on an orbital shaker at low speed for 30 min. After digestion, the single-cell suspension was filtered through a 100 m strainer, washed, and cultured in tissue culture flasks containing media from NeuroCult NS-A Human Proliferation Kit (StemCell Technologies, Cambridge, Mass.) supplemented with 0.0200 Heparin (StemCell Technologies), 20 ng/ml hEGF (Miltenyi Biotec, Cambridge, Mass.) and 20 ng/ml hFGF-2 (Miltenyi Biotec). Established cell lines were tested mycoplasma free (Venor™ Mycoplasma Detection Kit, Sigma Aldrich) and verified as MCC through immunohistochemical staining using antibodies against CK20 and SOX2.

b. Cell Culture Optimization

Cell lines were authenticated as MCC through immunohistochemical staining using antibodies against CK20 and SOX2 as follows:

| | | Cell | MCPyV | | | History of |
|---|---|---|---|---|---|---|
| | | Line | Viral | | Response to | immune |
| Patient | Gender | Source | Status | Prior Treatment | PD1:PD-L1 | suppression |
| 277 | M | PDX | Virus- | CE; chemoradiation; MLN0128; | CR | — |
| | | | positive | CAV; octreotide; imiquimod; | | |
| | | | | cabozantinib | | |
| 282 | M | PDX | Virus- | XRT | — | heart transplant |
| | | | negative | | | |
| 290 | F | PDX | Virus- | — | — | — |
| | | | negative | | | |
| 301 | M | PDX | Virus- | CE, chemoradiation | PD | — |
| | | | positive | | | |
| 320 | M | PDX | Virus- | CE; chemoradiation | — | — |
| | | | negative | | | |
| 336 | F | Tumor | Virus- | CE, chemoradiation | — | — |
| | | | positive | | | |
| 350 | M | Tumor | Virus- | XRT | PD | — |
| | | | negative | | | |
| 358 | F | Tumor | Virus- | XRT | Discontinued due | rheumatoid |
| | | | positive | | to side effects | arthritis on |
| | | | | | | adalimumab |
| 367 | M | PDX | Virus- | XRT | — | — |
| | | | positive | | | |
| 383 | M | Tumor | Virus- | XRT | adjuvant | — |
| | | | positive | | | |
| 2314 | F | PDX | Virus- | Everolimus; CE; Paclitaxel | — | — |
| | | | positive | | | |

Cell lines were authenticated as derivatives of original tumor samples by HLA typing for 7 of 11 lines as follows:

| | HLA | | | | |
|---|---|---|---|---|---|
| Patient | Allele | Tumor | | Cell Line | |
| MCC-277 | HLA-A | HLA-A*11:01:01 | HLA-A*32:01:01 | HLA-A*11:01:01 | HLA-A*32:01:01 |
| | HLA-B | HLA-B*14:01:01 | HLA-B*51:01:01 | HLA-B*14:01:01 | HLA-B*51:01:01 |
| | HLA-C | HLA-C*15:02:01 | HLA-C*08:02:01 | HLA-C*15:02:01 | HLA-C*08:02:01 |
| MCC-301 | HLA-A | HLA- | HLA- | HLA- | HLA- |
| | | A*24:02:01:01 | A*02:01:01:01 | A*24:02:01:01 | A*02:01:01:01 |
| | HLA-B | HLA-B*15:18:01 | HLA-B*44:02:01:01 | HLA-B*15:18:01 | HLA-B*44:02:01:01 |
| | HLA-C | HLA-C*07:04:01 | HLA-C*05:01:01:02 | HLA-C*07:04:01 | HLA-C*05:01:01:02 |
| MCC- 320 | HLA-A | HLA- | HLA-A*25:01:01 | HLA- | HLA-A*25:01:01 |
| | | A*01:01:01:01 | | A*01:01:01:01 | |
| | HLA-B | HLA-B*14:01:01 | HLA-B*18:01:01:02 | HLA-B*14:01:01 | HLA-B*18:01:01:02 |
| | HLA-C | HLA-C*12:03:01:01 | HLA-C*08:02:01 | HLA-C*12:03:01:01 | HLA-C*08:02:01 |
| MCC-336 | HLA-A | HLA- | HLA- | HLA- | HLA- |
| | | A*02:01:01:01 | A*02:01:01:01 | A*02:01:01:01 | A*02:01:01:01 |
| | HLA-B | HLA-B*35:02:01 | HLA-B*52:01:01:02 | HLA-B*35:02:01 | HLA-B*52:01:01:02 |
| | HLA-C | HLA-C*12:02:02 | HLA-C*04:01:01:01 | HLA-C*12:02:02 | HLA-C*04:01:01:01 |
| MCC-350 | HLA-A | HLA- | HLA- | HLA- | HLA- |
| | | A*24:02:01:01 | A*29:02:01:01 | A*24:02:01:01 | A*29:02:01:01 |
| | HLA-B | HLA-B*07:02:01 | HLA-B*08:01:01 | HLA-B*07:02:01 | HLA-B*08:01:01 |
| | HLA-C | HLA-C*07:02:01:01 | HLA-C*07:01:01:01 | HLA-C*07:02:01:01 | HLA-C*07:01:01:01 |
| MCC-367 | HLA-A | HLA- | HLA-A*31:01:02 | HLA- | HLA-A*31:01:02 |
| | | A*01:01:01:01 | | A*01:01:01:01 | |
| | HLA-B | HLA-B*49:01:01 | HLA-B*51:01:01 | HLA-B*49:01:01 | HLA-B*51:01:01 |
| | HLA-C | HLA-C*12:03:01:01 | HLA-C*01:02:01 | HLA-C*12:03:01:01 | HLA-C*01:02:01 |
| MCC-2314 | HLA-A | HLA- | HLA- | HLA- | HLA- |
| | | A*24:02:01:01 | A*02:01:01:01 | A*24:02:01:01 | A*02:01:01:01 |
| | HLA-B | HLA-B*07:02:01 | HLA-B*44:02:01:01 | HLA-B*07:02:01 | HLA-B*44:02:01:01 |
| | HLA-C | HLA-C*07:02:01:03 | HLA-C*05:01:01:02 | HLA-C*07:02:01:03 | HLA-C*05:01:01:02 |

All MCC cell lines were maintained in media from NeuroCult NS-A Proliferation Kit supplemented with 0.02% heparin, 20 ng/mL hEGF, 20 ng/mL hFGF2. Other media used for cell culture optimization included Stemflex (Gibco, Dublin, Ireland), Neurobasal (Gibco), and DMEM GlutaMAX (Gibco) with supplements as detailed herein. K562 cells were kept in DMEM GlutaMAX supplemented with 10% fetal bovine serum, 1% penicillin/streptomycin, HEPES, 3-mercaptoethanol, sodium pyruvate (all from Gibco). Media used for cell culture optimization were NeuroCult NS-A Proliferation Kit (StemCell Technologies), StemFlex, Neurobasal (Gibco) supplemented with 0.02% heparin (StemCell Technologies), 20 ng/mL hEGF (Miltenyi Biotec), 20 ng/mL hFGF2 (Miltenyi Biotec), and DMEM GlutaMAX (Gibco) supplemented with 10% FBS (Gibco), 1% penicillin/streptomycin (Gibco), 1 mM sodium pyruvate (Life Technologies), 10 mM HEPES (Life Technologies), and 55 nM 3-mercaptoethanol (Gibco).

c. Histology

Up to 10 million MCC cells were fixed in 10% formaldehyde. Cell pellets were washed with PBS and mounted on a paraffin block. 5 μm sections were cut and stained.

d. Flow Cytometry

Cells were dissociated with Versene and incubated with 5 μL Human TruStain FcX (Fc receptor blocking solution; Biolegend, Dedham, Mass.) per million cells in 100 mL at room temperature for 10 min. Fluorochrome-conjugated antibodies or respective isotype controls were immediately added and incubated for another 30 min at 4° C. Cells were then washed once with PBS and resuspended in PBS containing 4% paraformaldehyde and analyzed on LSR Fortessa cytometers. Additional steps were described for individual experiments as below. For upregulation of HLA Class I experiment, 5×10⁵ MCC cells were treated with increasing doses of IFNα2b, IFN3, IFNγ for 24 hours, or MEK inhibitors and DMSO for 72 hours before staining with W6/32 and Live/Dead as above.

e. Immunoprecipitation and Mass Spectrometry Analysis

Up to 40 million MCC cells were immunoprecipitated. Briefly, MCC cells were harvested and lysed in ice-cold lysis buffer containing 40M Tris (pH 8.0), 1 mM EDTA (pH 8.0), 0.1M sodium chloride, Triton X-100, 0.06M octyl 3-d-glucopyranoside, 100 U/mL DNAse I, 1 mM phenylmethanesulfonyl fluoride (all from Sigma Aldrich), protease inhibitor cocktail (Roche Diagnostics, Indianapolis, Ind.). Cell lysates were centrifuged at 12,700 rpm at 4° C. for 22 min. Lysate supernatants were coupled with Gammabind Plus sepharose beads (GE Healthcare) and incubated with 10 g of HLA Class I (Clone W6/32, Santa Cruz Biotechnologies) or HLA-E (Clone 3D12, eBiosciences, San Diego, Calif.) at 4° C. under rotary agitation for 3 h. After incubation, lysate-bead-antibody mixtures were briefly centrifuged and supernatants were discarded. Beads were washed with lysis buffer without protease inhibitors, wash buffer containing 40 mM Tris (pH 8.0), 1 mM EDTA (pH 8.0), 0.1 M sodium chloride, 0.06 M octyl 3-d-glucopyranoside, and 20 mM Tris buffer. Gel loading tips (Fisherbrand, FisherScientific, Pittsburgh, Pa.) were used to remove as much fluids from beads as possible. Peptides of up to three IPs were combined, acid eluted, and analyzed using LC/MS/MS as described previously (Abelin, Keskin Immunity). Briefly, peptides were resuspended in 3% ACN, 5% FA and loaded onto an analytical column (20-30 cm, 1.9 μm C18 Dr. Maisch, packed in-house). Peptides were eluted in a 6-30% gradient (EasyLC 1000 or 1200, ThermoFisher Scientific) and analyzed on a QExactive Plus or Fusion Lumos (ThermoFisher Scientific). For Lumos measurements, peptides were also subjected to fragmentation if they were singly charged.

For detection of the large T antigen peptide, 3 Ips of a 367 Cell line treated with IFNγ were pooled, acid eluted, fractionated using stage tip basic reverse phase separation and fractions were analyzed on a Fusion Lumos equipped with a FAIMSpro interface (Klaeger et al., in preparation).

Mass spectra were interpreted using Spectrum Mill software package v7.1 pre-Release (Agilent Technologies, Santa Clara, Calif.). MS/MS spectra were excluded from searching if they did not have a precursor MH+ in the range of 600-4000, had a precursor charge >5, or had a minimum of <5 detected peaks. Merging of similar spectra with the same precursor m/z acquired in the same chromatographic peak was disabled. MS/MS spectra were searched against a protein sequence database that contained 98,298 entries, including all UCSC Genome Browser genes with hg19 annotation of the genome and its protein coding transcripts (63,691 entries), common human virus sequences (30,181 entries), recurrently mutated proteins observed in tumors from 26 tissues (4,167 entries), 264 common laboratory contaminants as well as protein sequences containing somatic mutations detected in MCC cell lines. MS/MS search parameters included: no-enzyme specificity; fixed modification: carbamidomethylation of cysteine; variable modifications: oxidation of methionine, and pyroglutamic acid at peptide N-terminal glutamine; precursor mass tolerance of 10 ppm; product mass tolerance of ±10 ppm, and a minimum matched peak intensity of 30%. Peptide spectrum matches (PSMs) for individual spectra were automatically designated as confidently assigned using the Spectrum Mill autovalidation module to apply target-decoy based FDR estimation at the PSM level of <1% FDR. Peptide auto-validation was done separately for each sample with an auto thresholds strategy to optimize score and delta Rank1-Rank2 score thresholds separately for each precursor charge state (1 thru 4) across all LC-MS/MS runs per sample. Score threshold determination also required that peptides had a minimum sequence length of 7, and PSMs had a minimum backbone cleavage score (BCS) of 5. Peptide and PSM exports were filtered for contaminants including potential carry over tryptic peptides and peptides identified in a blank bead sample. For a fairer comparison of IFNγ+/−samples, PSMs were filtered by rawfiles that resembled similar cell numbers and IP input for both conditions.

f. Whole Proteome Analysis and Interpretation

Protein expression of MCC cell lines was assessed as described previously (Mertins et al. (2018) Nature Protocols 13 (7): 1632-61. Briefly, cell pellets of MCC cell lines with and without IFNγ treatment were lysed in 8M Urea and digested to peptides using LysC and Trypsin (Promega). 400 μg peptides were labeled with TMT10 reagents (Thermo Fisher, 126-MCC290, 127N-MCC350_IFN, 127C MCC275_IFN, 128N MCC275, 128C MCC350, 129N_MCC301_IFN, 129C-MCC277, 130N-MCC290_IFNy, 130C MCC277 IFN, 131 MCC301) and then pooled for subsequent fractionation and analysis. Pooled peptides were separated into 24 fractions using offline high pH reversed phase fractionation. 1 μg per fraction was loaded onto an analytical column (20-30 cm, 1.9 μm C18 Reprosil beads (Dr. Maisch HPLC GmbH), packed in-house PicoFrit 75 μM inner diameter, 10 μM emitter (New Objective)). Peptides were eluted with a linear gradient (EasyNanoLC 1000 or 1200, Thermo Scientific) ranging from 6-30% Buffer B (either 0.1% FA or 0.5% AcOH and 80% or 90% ACN) over 84 min, 30-90% B over 9 min and held at 90% Buffer B for 5 min at 200 nl/min. During data dependent acquisition, peptides were analyzed on a Fusion Lumos (Thermo Scientific). Full scan MS was acquired at a 60,000 from 300-1,800 m/z. AGC target was set to 4e5 and 50 ms. The top 20 precursors per cycle were subjected to HCD fragmentation at 60,000 resolution with an isolation width of 0.7 m/z, 34 NCE, 3e4 AGC target and 50 ms max injection time. Dynamic exclusion was enabled with a duration of 45 sec.

Spectra were searched using Spectrum Mill against the database described above excluding MCC variants, specifying Trypsin/allow P (allows K—P and R—P cleavage) as digestion enzyme and allowing 4 missed cleavages. Carbamidomethylation of cysteine was set as a fixed modification. TMT labeling was required at lysine, but peptide N-termini were allowed to be either labeled or unlabeled. Variable modifications searched include acetylation at the protein N-terminus, oxidized methionine, pyroglutamic acid, deamidated asparagine and pyrocarbamidomethyl cysteine. Match tolerances were set to 20 ppm on MS1 and MS2 level. PSMs score thresholding used the Spectrum Mill auto-validation module to apply target-decoy based FDR in 2 steps: at the peptide spectrum match (PSM) level and the protein level. In step 1 PSM-level autovalidation was done first using an auto-thresholds strategy with a minimum sequence length of 8; automatic variable range precursor mass filtering; and score and delta Rank1-Rank2 score thresholds optimized to yield a PSM-level FDR estimate for precursor charges 2 through 4 of <1.0% for each precursor charge state in each LC-MS/MS run. To achieve reasonable statistics for precursor charges 5-6, thresholds were optimized to yield a PSM-level FDR estimate of <0.5% across all LC runs per experiment (instead of per each run), since many fewer spectra are generated for the higher charge states. In step 2, protein-polishing autovalidation was applied to each experiment to further filter the PSMs using a target protein-level FDR threshold of zero, the protein grouping method expand subgroups, top uses shared (SGT) with an absolute minimum protein score of 9.

g. ORF Screen

The human ORFeome version 8.1 lentiviral library, which contains 16,172 unique ORFs mapping to 13,833 genes, was supplied as a gift from the Broad Genetic Perturbations Platform. 75 million MCC301VP cells were transduced with ORFeome lentivirus to achieve an infection rate of 30%-40%. Two days later, transduced cells were selected with three days of 0.5 μg/mL puromycin treatment. Between 7-10 days after transduction, cells were stained with an anti-HLA-ABC-PE antibody (W6/32 clone, Biolegend #311405) and sorted on a BD FACSAria II, gating for the top and bottom 10% of HLA-ABC-PE staining. Subsequently, genomic DNA containing stably integrated ORF sequences was isolated from the sorted cells. The screen was performed in triplicate. Isolated genomic DNA was then used as a template for indexed PCR amplification of the construct barcode region. Pooled PCR products were purified and run on an Illumina HiSeq.

h. Generation of a Genome-Wide CRISPR-KO Lentiviral Library

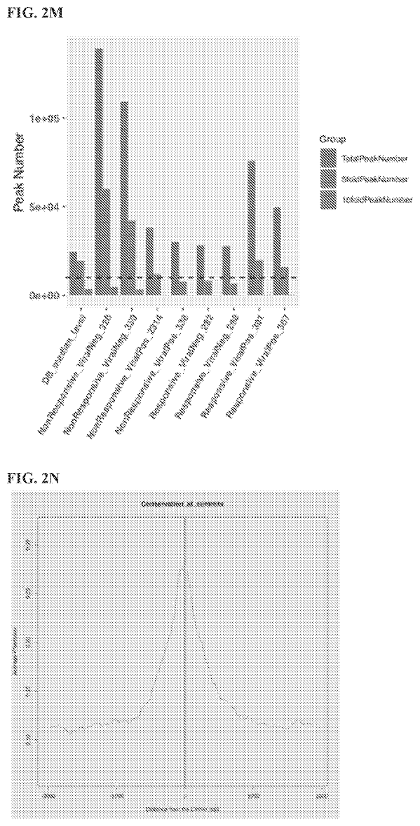

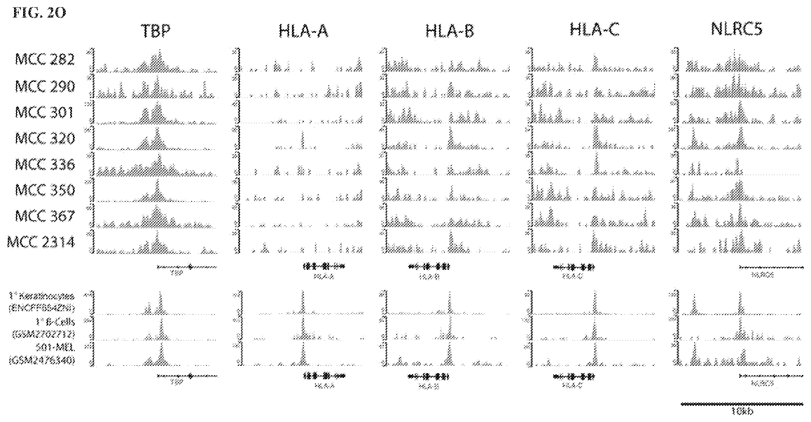

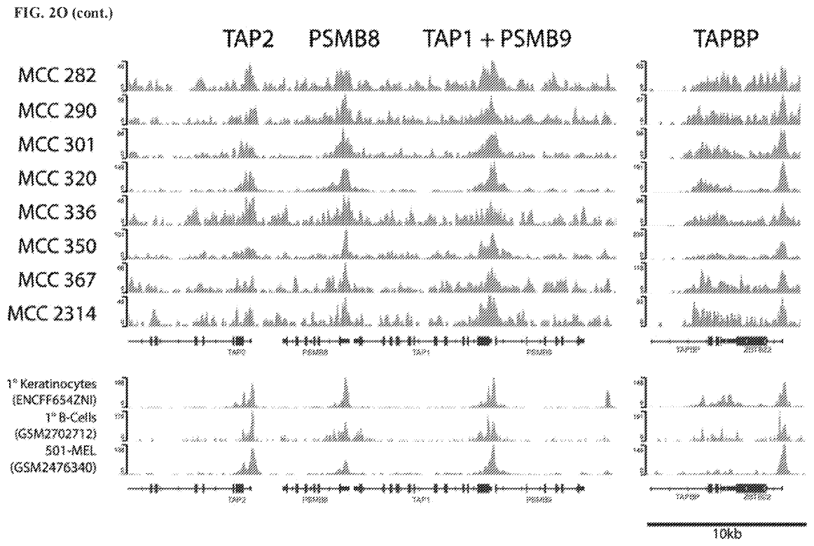

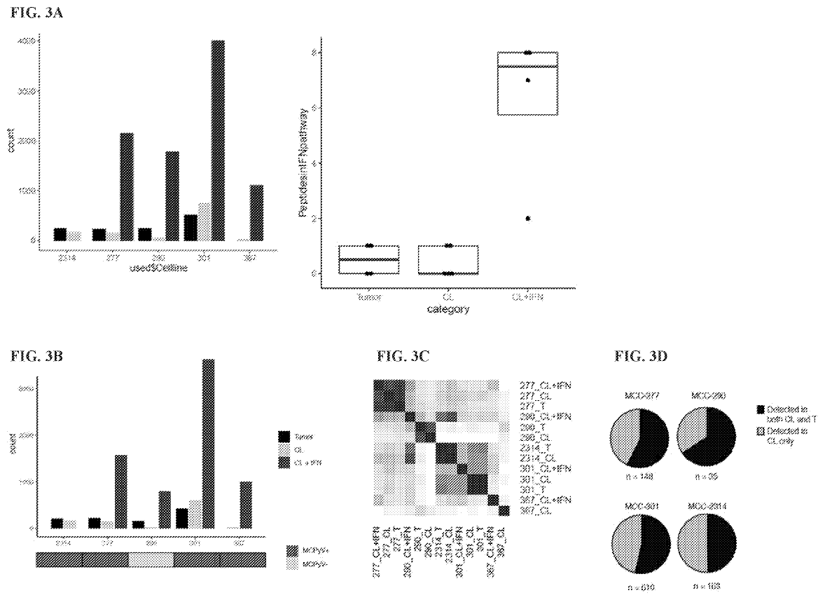

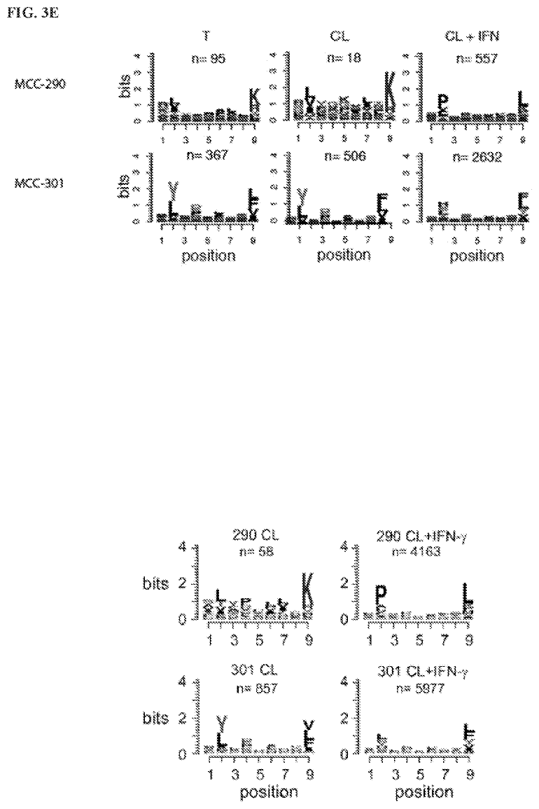

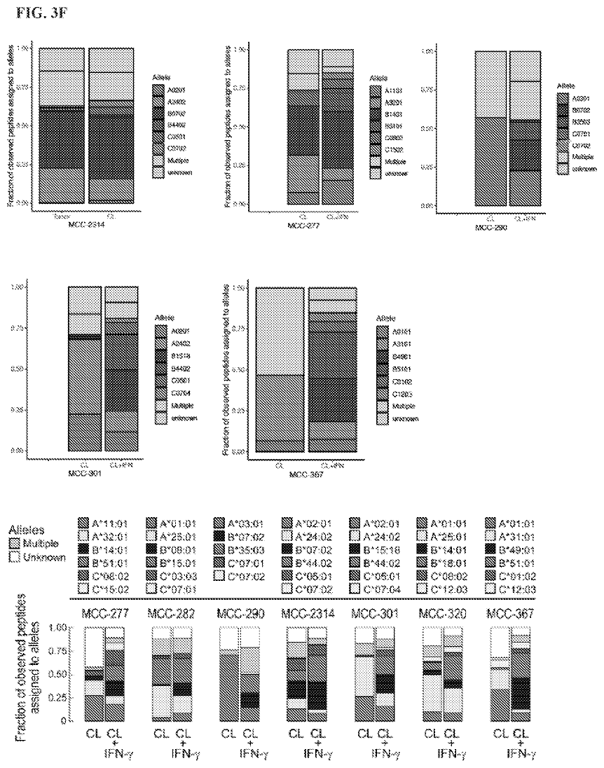

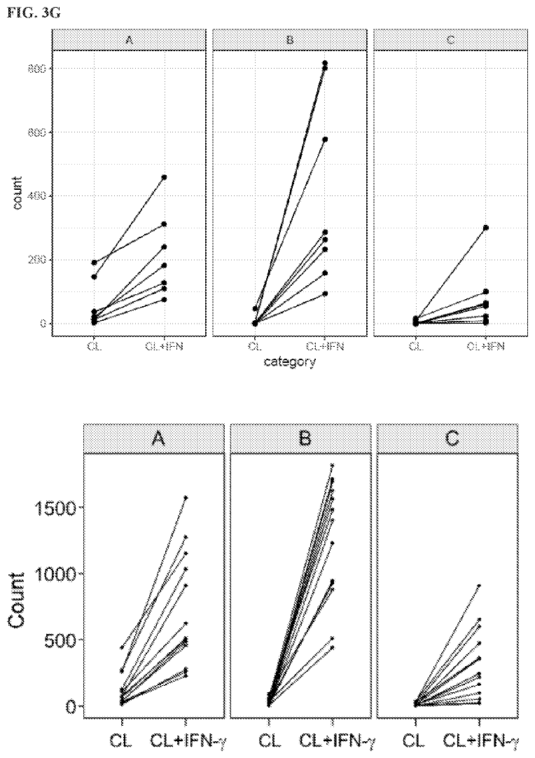

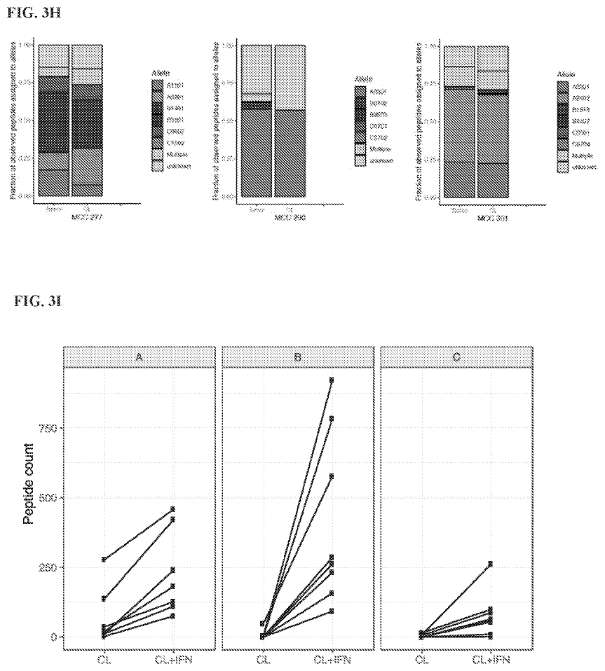

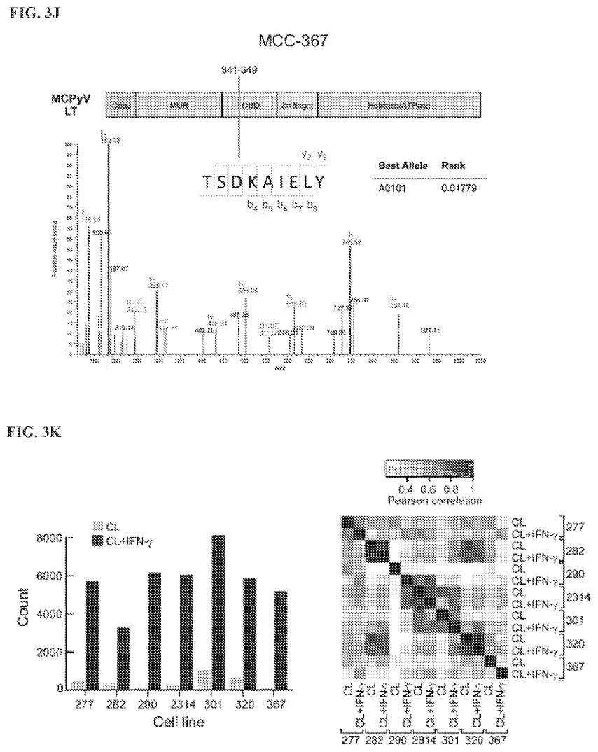

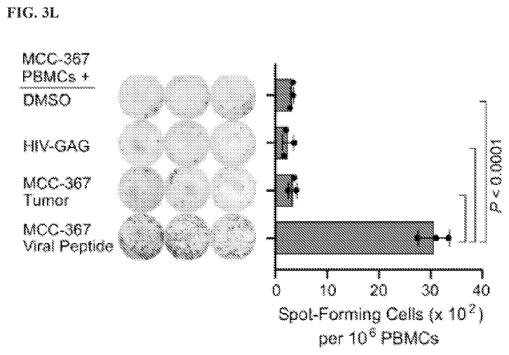

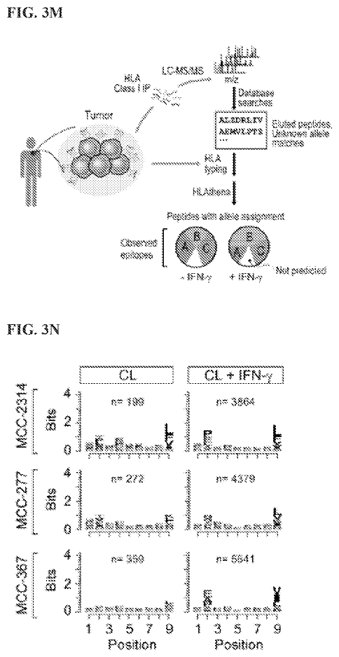

The Brunello human CRISPR knockout pooled plasmid library (Doench et al. (2016) Nature 34 (2): 184-91) (1-vector system) was a gift from David Root and John Doench (Watertown, Mass., Addgene #73179). Fifty ng of the Brunello human CRISPR knockout pooled plasmid library (1-vector system) was electroporated into ElectroMAX Stbl4 competent cells (ThermoFisher, Cat. No. #11635018) and incubated overnight at 30° C. on 24.5×24.5 cm agar bioassay plates. 20 hours later, colonies were harvested and pooled, and the amplified plasmid DNA (pDNA) was extracted and purified. To confirm library diversity was maintained after amplification, sgRNA barcode construct regions were PCR amplified in pre- and post-amplification library aliquots. PCR products were purified and sequenced on an Illumina MiSeq. Sequencing data from pre- and post-amplification aliquots were compared to ensure similar diversity (FIG. 3A). To produce lentivirus, HEK-293T cells were transfected with pDNA, VSV.G, and psPAX2 plasmids using the TransIT-LT1 transfection reagent (Mirus Bio, Madison, Wis., Cat. No. #MIR2300). Lentivirus was harvested 48 hours post-transfection and flash frozen. To titer lentivirus, 1.5 million cells MCC-301 cells were transduced with 100, 200, 300, 500, and 700 μL of virus. From each condition, half of the cells were selected with 0.5 μg/mL puromycin. Infection rates were calculated by comparing live cell counts in selected and unselected conditions.

i. CRISPR-KO Screen

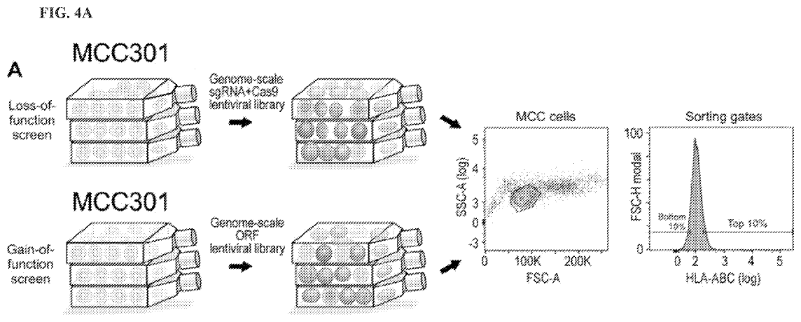

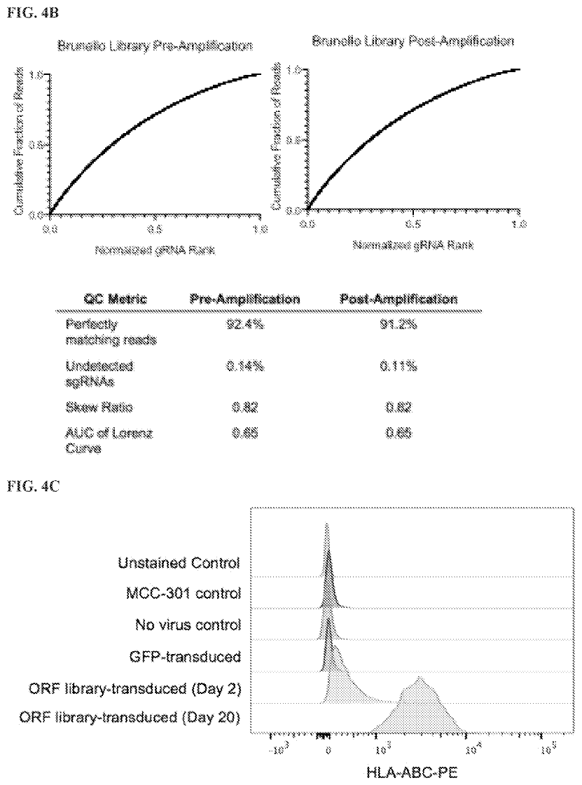

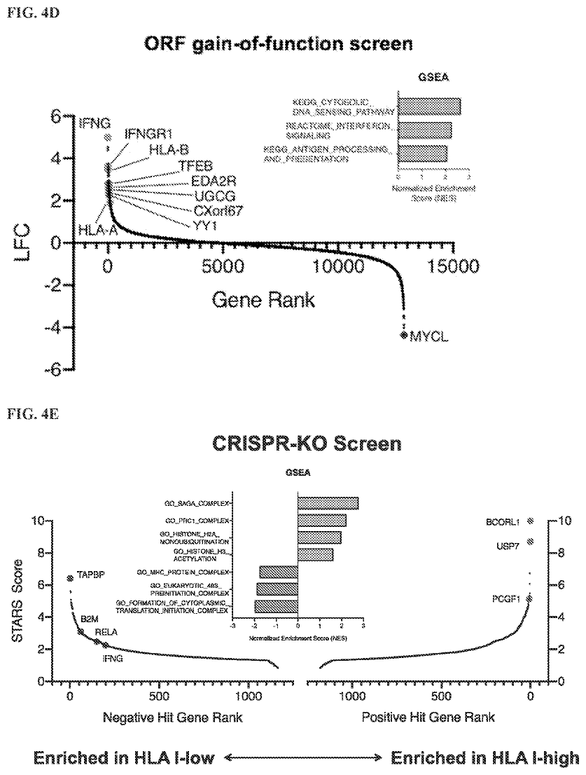

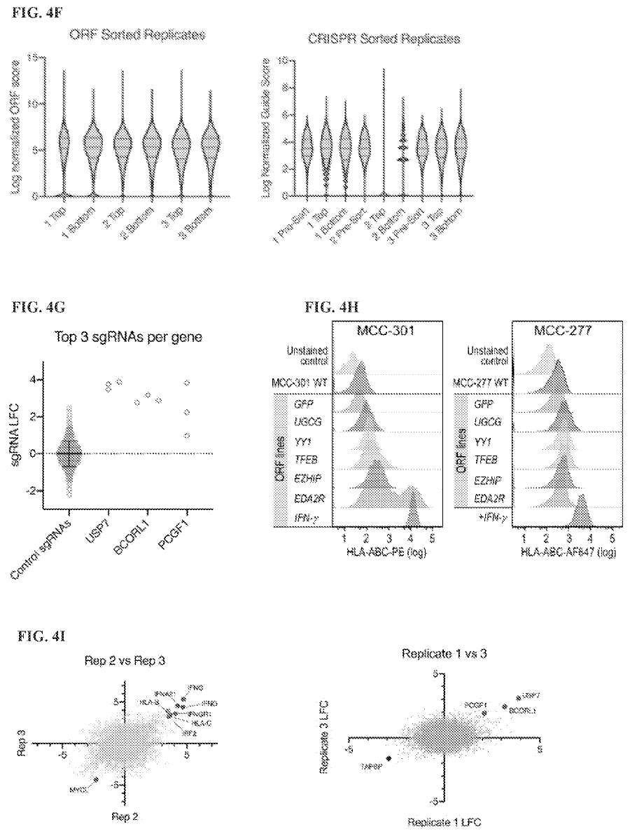

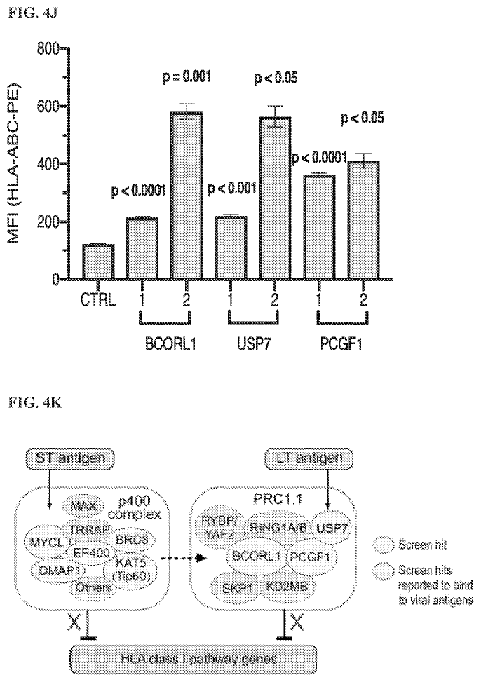

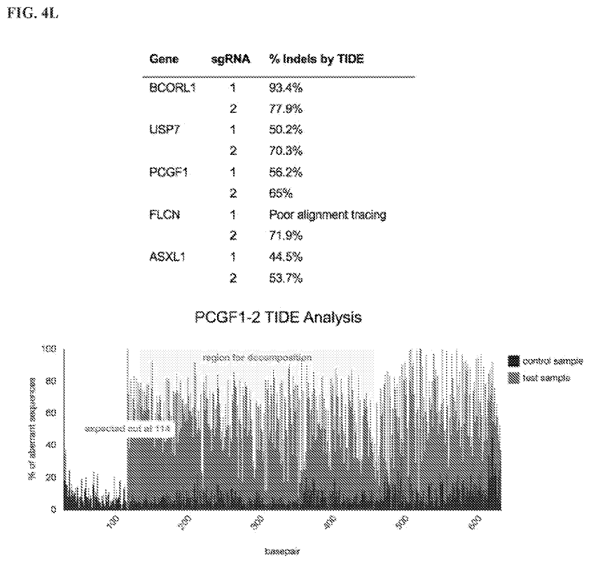

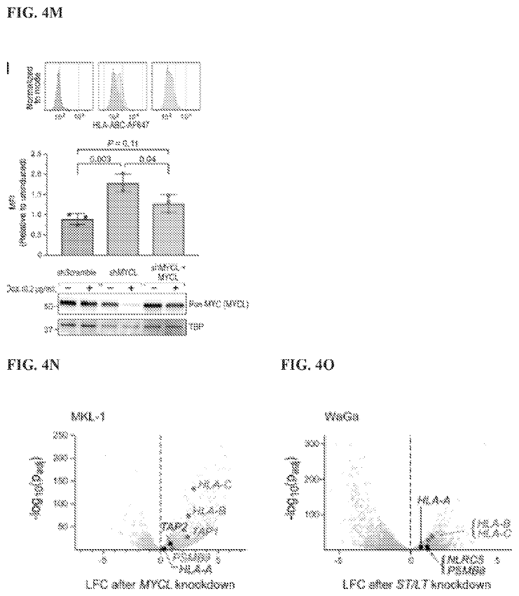

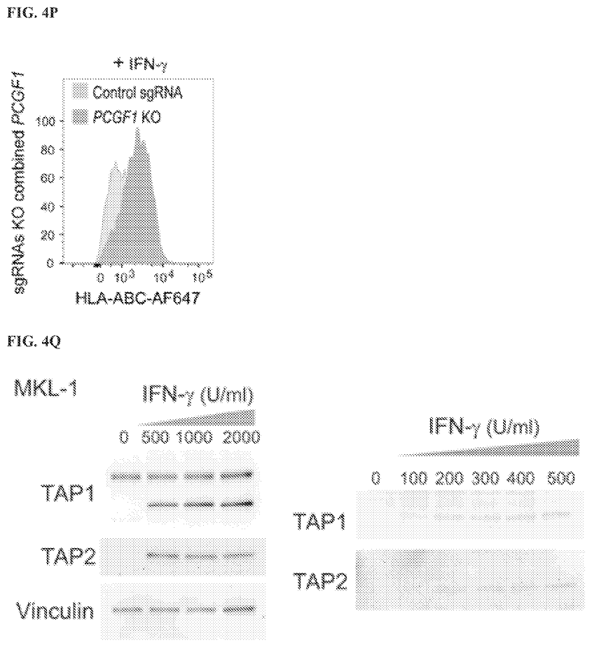

The human Brunello CRISPR knockout pooled plasmid library, which contains 76,441 sgRNAs targeting 19,114 genes, was a gift from David Root and John Doench (Addgene, Cat. No. #73178). The Brunello plasmid library was then transformed and propagated in electrocompetent Stbl4 cells, and lentivirus was produced in HEK-293T cells. Subsequent transduction and FACS screening were performed in triplicate analogously to the ORF screen with the following exceptions: 150 million MCC301VP cells were transduced per replicate, and cells were sorted days after transduction. Additionally, a representative pellet (40 million cells) after transduction but before flow cytometry selection was harvested and sequenced from all three replicates to assess sgRNA representation (FIG. 4F).

j. Screen Data Analysis

Unprocessed FASTQ reads were converted to log-normalized scores for each construct using the PoolQ software (Broad Institute). For each of the three replicates, log-fold changes (LFCs) between the top and bottom 10% scores were calculated for each construct. For the ORF screen, ORF constructs were then ranked based on their median LFC values, and corresponding p values were calculated using a hypergeometric distribution model (Broad Institute). For the CRISPR screen, replicate 2 was discarded due to poor sample quality, as measured by average sgRNA representation (FIG. 4C), and LFC values for each sgRNA were averaged between replicate 1 and 3 and then input into the STARS software (v1.3, Broad Institute) (Doench et al. (2016) supra), which employs a binomial distribution model to rank genes based on the ranks of their corresponding individual sgRNAs.

k. Generation of CRISPR KO Lines

Forward and reverse oligos with the sequence 5′ CACCG----sgRNA sequence---3′ and 5′ AAAC---reverse complement of sgRNA---C 3′ were synthesized by Eton Biosciences (San Diego, Calif.). Forward and reverse oligos were annealed and phosphorylated, producing BsmBI-compatible overhangs. LentiCRISPRv2 vector (Addgene, Cat. No. #52961) was digested with BsmBI, dephosphorylated with shrimp alkaline phosphatase, and gel purified. Vector and insert were ligated at a 1:8 ratio with T7 DNA ligase at room temperature and transformed into Stbl3 cells. Correct sgRNA cloning was confirmed via Sanger sequencing using the following primer: 5′-GATACAAGGCTGTTAGAGAGATAATT-3′. Lentivirus was produced and MCC-301 cells were transduced with single construct lentivirus in the same manner as for the CRISPR-KO library.

### 1. Whole Exome Sequencing and Mutation Calling

Genomic DNA Samples were sheared using a Broad Institute-developed protocol optimized for ˜180 bp size distribution. Kapa Hyperprep kits were used to construct libraries in a process optimized for somatic samples, including end repair, adapter ligation with forked adaptors containing unique molecular indexes and addition of P5 and P7 sample barcodes via PCR. SPRI purification was performed and resulting libraries were quantified with Pico Green. Libraries were normalized and equimolar pooling was performed to prepare multiplexed sets for hybridization. Automated capture was performed, followed by PCR of the enriched DNA. SPRI purification was used for cleanup. Multiplex pools were then quantified with Pico Green and DNA fragment size was estimated using Bioanalyzer. Final libraries were quantitated by qPCR and loaded onto an Illumina flowcell across an adequate number of lanes to achieve >=85% of target bases covered at >=50× depth, with a range from 130-160× mean coverage of the targeted region.

Exome-sequencing bam files were downloaded from the Broad Genomics Firecloud/Terra platform using the Google Cloud Storage command line tool gsutil version 4.5 (github.com/GoogleCloudPlatform/gsutil/). gatk version 4.1.2.0 (do Valle et al. (2016) BMC Bioinformatics 17 (12): 27-35) was used to: (1) call mutations from reference on normal bams with Mutect2 command (Benjamin et al. (2019) bioRxiv, doi.org/10.1101/861054) using a max MNP distance of 0, (2) build a panel of normals from vcf files of called normal mutations using the CreateSomaticPanelOfNormals command, and (3) call mutations between pairs of both tumor and cell line with compared to their respective normal counterpart using the Mutect2 command. For these steps, the following annotations were used: b37 reference sequence downloaded from ftp.broadinstitute.org/bundle/b37/human_g1k_v37.fasta, germline resource vcf downloaded from ftp.broadinstitute.org/bundle/beta/Mutect2/af-only-gnomad.raw.sites.b37.vcfgz, and intervals list downloaded from github.com/broadinstitute/gatk/blob/master/src/test/resources/large/whole_exome_illumina_coding_v1.Homo_sapiens_assembly19.targets.interval_list. Called variants were filtered with the gatk FilterMutectCalls command, and variants labeled as PASS were extracted and included in downstream analyses.

Next, vcf files of passing variants were annotated as maf files using vcf2maf version 1.16.17 (downloaded from github.com/mskcc/vcf2maf/tree/5453f802d2f1f261708fe21c9d47b66d13e19737) and Variant Effect Predictor (VEP) version 95 installed from github.com/Ensembl/ensembl-vep/archive/release/95.3.tar.gz (McLaren et al. (2016) Genome Biology 17 (1): 1-14). R Bioconductor package maftools (Mayakonda et al. (2018) Genome Research 28 (11): 1747-56) were used to generate oncoplots of mutations by gene and sample.

m. Whole Genome Sequencing and Copy Number Analysis

Whole genome sequencing was performed by Admera Health. Reads were quality and adapter trimmed using TrimGalore with default settings. Trimmed reads were aligned against a fusion reference containing hg38 and MCPyV (NCBI accession number: NC_010277) using bowtie2-very-sensitive. Copy number variant analysis was performed with GATK4 CNV recommended practices. A panel of normals was generated from 17 normal blood whole genomes to call CNVs from tumors.

n. RNA Sequencing Analysis

RNA samples were first assessed for quality using the Agilent Bioanalyzer (DV200 metric). One hundred ng of RNA were used as the input for first strand cDNA synthesis using Superscript III reverse transcriptase and Illumina's TruSeq RNA Access Sample Prep Kit. Synthesis of the second strand of cDNA was followed by indexed adapter ligation with UMI (unique molecular index) adaptors. Subsequent PCR amplification enriched for adapted fragments. Amplified libraries were quantified, normalized, pooled, and hybridized with exome targeting oligos. Following hybridization, bead clean-up, elution and PCR was performed to prepare library pools for sequencing on Illumina flowcell lanes. Transcriptomes were sequenced to a coverage of at least 50 million reads in pairs.

Raw fastq files for fibroblast and keratinocyte control lines were downloaded from SRA using R Bioconductor package SRAdb (Mayakonda et al. (2018) supra; Zhu et al. (2013) BMC Bioinformatics 14 (1): 1-4) using accession codes SRP126422 (4 replicates from control samples ‘NN’) and SRP131347 (6 replicates with condition: control and genotype: control). These fastq files, along with those for the mkl1 and waga cell lines, were aligned using STAR version 2.7.3a (Dobin et al. (2013) Bioinformatics 29 (1): 15-21), using index genome reference file downloaded from ftp.ebi.ac.uk/pub/databases/gencode/Gencode_human/release_19/GRCh37.p13.genome.fa.gz, transcript annotation file downloaded from data.broadinstitute.org/snowman/hg19/star/gencode.v19.annotation.gtf, and with the following options: --twopassMode Basic, --outSAMstrandField intronMotif, --alignIntronMax 1000000, --alignMatesGapMax 1000000, --sjdbScore 2, --outSAMtype BAM Unsorted, --outSAMattributes NH HI NM MD AS XS, --outFilterType BySJout, --outSAMunmapped Within, --genomeLoad NoSharedMemory, --outFilterScoreMinOverLread 0, --outFilterMatchNminOverLread 0, --outFilterMismatchNmax 999, and outFilterMultimapNmax 20. Duplicates were marked with picard MarkDuplicates version 2.22.0-SNAPSHOT.

RNA-sequencing bam files for MCC tumor and cell line samples were downloaded from the Broad Genomics Firecloud/Terra platform using the Google Cloud Storage command line tool gsutil version 4.5 (github.com/GoogleCloudPlatform/gsutil/).

Gene counts were obtained from bam files using featureCounts version 2.0.0 (doi.org/10.1093/bioinformatics/btt656). Very lowly expressed genes with average count across samples less than 1 were excluded from analysis. Between-sample distance metrics were computed using the Euclidean distance on the vectors of variance-stabilized counts obtained from the vst function in the DESeq2 R Bioconductor package (Dobin et al. (2013) supra; Love et al. (2014) Genome Biology 15 (12): 550).

Differential expression analysis was carried out between (1) viral positive and viral negative samples (adjusting for cell line or tumor as a covariate), (2) cell line and tumor samples (adjusting for viral status as a covariate), and (3) IFNγ plus and minus samples (adjusting for viral status as a covariate) using the negative binomial GLM Wald test of DESeq2, where significance was assessed using the p-values adjusted for multiple comparisons under default settings. To account for potential global gene expression differences among sample groups, RUVg (Risso et al. (2014) Nature 32 (9): 896-902) was used to estimate latent factors of unwanted variation from the list of housekeeping genes downloaded from www.tau.ac.il/˜elieis/HKG/HK_genes.txt. The largest factor of unwanted variation was then used as a covariate in the DESeq2 models to adjust for latent variation unrelated to library size. Gene set enrichment analyses were carried out on the differential expression results described above using the fgsea R Bioconductor package (Korotkevich et al. (2016) Bioinformatics. bioRxiv).

o. ATAC-Seq

ATAC sequencing was performed by Admera Health. ATAC sequencing was analyzed using the Kundaje lab ATAC seq pipeline (github.com/kundajelab/atac_dnase_pipelines) for paired end reads with hg38 as the reference. Peaks from the overlap of pseudo-replicates were used for downstream analysis. To confirm the quality of the ATAC-Seq data, each sample was benchmarked against publicly available ATAC-Seq datasets on Cistrome DB (Zheng et al. (2019) Nucleic Acids Research 47 (D1): D729-35) and by evaluating peak conservation.

p. Differential Peak Analysis

Differential ATAC-seq peaks between (1) viral positive and negative samples, and (2) IFNγ responsive and non-responsive (split into top four and bottom four <Patrick to fill in details>) were called using the DiffBind R Bioconductor package (Ross-Innes et al. (2012) Nature 481 (7381): 389-93). Significance was assessed using the using adjusted p-values from the negative binomial GLM Wald test of DESeq2, which is called by DiffBind. Peaks were annotated by the gene with the nearest TSS using the ChIPpeakAnno (Zhu et al. (2010) BMC Bioinformatics 11 (May): 237) and the TxDb.Hsapiens.UCSC.hg38.knownGene (TxDb.Hsapiens.UCSC.hg19.knownGene) R Bioconductor packages. Comparison ATAC-Seq datasets for visualization were retrieved from GEO (GSM2702712—primary B-cells; GSM2476340—501MEL cell line) and ENCODE (ENCFF654ZNI—primary fetal foreskin keratinocyte). To visualize ATAC-Seq tracks, all BAM files were normalized identically using bamCoverage from deepTools (academic.oup.com/nar/article/44/W1/W160/2499308) with a 10 nucleotide bin size and normalization method of reads per kilobase of transcript per million reads (RPKM). Resulting bigwig files were visualized in the Integrative Genome Viewer (www.ncbi.nlm.nih.gov/pmc/articles/PMC3346182/).

q. Whole Genome Bisulfite Sequencing

Whole genome bisulfite sequencing (WGBS) was performed by Admera Health. The hg38 reference genome was prepared using Bismark .Reads were aligned to the prepared hg38 genome, deduplicated, and methylation states were extracted using Bismark with default settings.

Bismark methylation count output files (.cov) were strand-collapsed using the bsseq Bioconductor package (Hansen et al. (2012) Genome Biology 13 (10): R83). CpG sites covered by at least 1 read in fewer than 4 samples were excluded from further analysis. Promoter regions (2000 basepair upstream, 200 basepair downstream) of all transcripts annotated by the TxDb.Hsapiens.UCSC.hg38.knownGene (TxDb.Hsapiens.UCSC.hg19.knownGene) R Bioconductor package. Then, raw methylation levels (methylated counts divided by coverage) for all sites within each promoter region of all transcripts matching each gene symbol were averaged.

r. MCPyV Viral Transcript Detection (Nucleic Acid Isolation, Library Preparation and Sequencing)

To perform ViroPanel with and without supplementation with the OncoPanel (v3) bait set, purified DNA was quantified using a Quant-iT PicoGreen dsDNA assay (Thermo Fisher). (Starrett et al. (2020) Genome Medicine 12:30). Library construction was performed using 200 ng of DNA, which was first fragmented to ˜250 bp using a Covaris LE220 Focused ultrasonicator (Covaris, Woburn, Mass.) followed by size-selected cleanup using Agencourt AMPureXP beads (Beckman Coulter, Inc. Indianapolis, Ind.) at a 1:1 bead to sample ratio. Fragmented DNA was converted to Illumina libraries using a KAPA HTP library kit using the manufacturer's recommendations (Thermo Fisher). Adapter ligation was done using xGen dual index UMI adapters (IDT, Coralville, Iowa).

Samples were pooled in equal volume and run on an Illumina MiSeq nano flow cell to quantify the amount of library based on the number of reads per barcode. All samples yielded sufficient library (>250 ng) and were taken forward into hybrid capture. Libraries were pooled at equal mass (3×17-plex and 1×18-plex) to a total of 750 ng. Captures were done using the SureSelectXT Fast target enrichment assay (Agilent, Technologies, Santa Clara, Calif.) with ViroPanel with and without supplementation with the OncoPanel (v3) bait set. Captures were sequenced on an Illumina 2500 in rapid run mode (Illumina Inc., San Diego, Calif.).

A custom perl script was written to extract, assemble, annotate, and visualize viral reads and determine viral integration sites. Viral reads and their mates were first identified and extracted by those that have at least one mate map to the viral genome. Additional reads containing viral sequence were identified by a bloom filter constructed of unique, overlapping 31 bp k-mers of the MCPyV genome. The human genome positions for any read with a mate mapping to the viral genome were output into a bed file and the orientation of viral and human pairs was stored to accurately deconvolute overlapping integration sites. This bed file was then merged down into overlapping ranges based on orientation counting the number of reads overlapping that range. Skewdness in coverage of integration junctions was calculated by the difference in the fraction of virus-host read pairs overlapping the first and second halves of the aforementioned ranges. This skewdness value was used to determine the orientation of the viral-host junction (i.e., positive values, junction is on the 3′ end of the range; negative values,

junction is on the 5′ end of the range), which was validated from the results of de novo assembly. Integrated viral genomes were assembled from extracted reads using SPAdes with default parameters. The assembly graphs from SPAdes were annotated using blastn against hg19 and the MCPyV reference genome with an e-value cutoff of 1×10⁻¹⁰. Annotated assembly graphs were visualized using the ggraph R package.

Integration sites confirmed by reference guided alignment and assembly data were analyzed for stretches of microhomology between the human and viral genomes by selecting 10 bp upstream and downstream of the integration junction on the viral and human genomes. Within these sequences stretches of identical sequence at the same position longer than two base pairs were counted. Overall homology between the sequences was calculated by Levenshtein distance. Three integration junctions with indeterminate DNA sequence ranging from 1 to 25 bp inserted between viral and human DNA were excluded from analysis. Expected microhomology was calculated by randomly selecting 1000 20 bp pairs of non-N containing sequence from the human and MCPyV genomes.

Integration site proximity to repeat elements were determined using bedtools closest and repeatmasker annotations acquired from the UCSC genome browser. Expected frequency of integration near repeat elements was determined by randomly selecting 1000 sites in the human genome. Sites within 2 kb of a repeat element were counted as close proximity.

Functional annotation of somatic mutations and viral integration events was performed using PANTHER (www.pantherdb.org).

s. Viral Transcript Quantification of RNA-Seq

The Merkel cell polyomavirus reference sequence was downloaded from www.ebi.ac.uk/ena/data/view/EU375804&display=fasta. Unmapped reads were extracted from RNA-seq bam files of tumor and cell line using SAMtools view version 1.10 (Li et al. (2009) Bioinformatics 25 (16): 2078-79) and realigned to the MCC Polyomavirus reference sequence using bwa version 0.7.17-r1188 (Li and Durbin 2010). Finally, the number of reads for each sample successfully mapped to the MCC Polyomavirus reference were counted with SAMtools view.

t. Dependency Map Correlations

The DepMap 20Q2 CRISPR dependency data were downloaded from www.depmap.org/portal/download/. TP53 mutation status was assigned using the Cell-Line Selector tool on the DepMap Portal based on criteria of at least one encoding mutation. Pearson coefficients were calculating using test.cor in R, and two-sided p-values outputted by this function were converted into FDR using p.adjust. Plots were generated using ggplot2, tidyverse, gridExtra, cowplot, and scales. GSEA was performed using a gene list ranked by -log(p-val) multiplied by (−1) if the Pearson correlation was negative.

u. Quantification and Statistical Analysis

Specific software packages with version numbers, along with details of all statistical analyses are listed in the respective methods sections above. No randomization procedures or sample size calculations were carried out as part of the study. All analysis code including specific parameter settings for whole exome seq analysis, RNA-seq analysis, ATAC-seq differential peak analysis, MCPyV viral transcript detection, and WGBS promoter signal extraction are made available in a Github repository under an MIT license at github.com/kdkorthauer/MCC. All analyses in R were carried out using version 3.6.2.

v. Oligos, Primers, and Key Resources

| Oligo Name | Sequence | Notes |
|---|---|---|
| BCORL1-1 fwd | CACCGTCCCGCATCTGACAGCGCCG | Oligo for guide RNA cloning |
| BCORL1-1 rev | AAACCGGCGCTGTCAGATGCGGGAC | Oligo for guide RNA cloning |
| BCORL1-2 fwd | CACCGGGAGGCGGGATATATACCAG | Oligo for guide RNA cloning |
| BCORL1-2 rev | AAACCTGGTATATATCCCGCCTCCC | Oligo for guide RNA cloning |
| USP7-1 fwd | CACCGTTGATGACGACGTGGTGTCA | Oligo for guide RNA cloning |
| USP7-1 rev | AAACTGACACCACGTCGTCATCAAC | Oligo for guide RNA cloning |
| USP7-2 fwd | CACCGGGCAGTAGAACAGCTCGATG | Oligo for guide RNA cloning |
| USP7-2 rev | AAACCATCGAGCTGTTCTACTGCCC | Oligo for guide RNA cloning |
| PCGF1-1 fwd | CACCGCCACGAAGTAGCCGGCGCAT | Oligo for guide RNA cloning |
| PCGF1-1 rev | AAACATGCGCCGGCTACTTCGTGGC | Oligo for guide RNA cloning |
| PCGF1-2 fwd | CACCGGCTCATCATAGCGATAGTAG | Oligo for guide RNA cloning |
| PCGFl-2 rev | AAACCTACTATCGCTATGATGAGCC | Oligo for guide RNA cloning |
| CTRL-1 fwd | CACCGTGCGGCGTAATGCTTGAAAG | Oligo for guide RNA cloning |
| CTRL-1 rev | AAACCTTTCAAGCATTACGCCGCAC | Oligo for guide RNA cloning |
| CTRL-2 fwd | CACCGGGATTAATTCGCTAAATGAT | Oligo for guide RNA cloning |
| CTRL-2 rev | AAACATCATTTAGCGAATTAATCCC | Oligo for guide RNA cloning |
| B2M fwd | TCTCTGCTGGATGACGTGAG | qPCR primer |
| B2M rev | TAGCTGTGCTCGCGCTACT | qPCR primer |
| TAP1 fwd | TCAGGGCTTTCGTACAGGAG | qPCR primer |
| TAP1 rev | TCCGGAAACCGTGTGTACTT | qPCR primer |
| TAP2 fwd | ACTGCATCCTGGATCTCCC | qPCR primer |
| TAP2 rev | TCGACTCACCCTCCTTTCTC | qPCR primer |
| TAPBP fwd | ACCCTGGAGGTAGCAGGTCTTT | qPCR primer |
| TAPBP rev | AATCCTTGCAGGTGGACAGGTAG | qPCR primer |
| LMP2 fwd | TCAAACACTCGGTTCACCAC | qPCR primer |
| LMP2 rev | GGAGAAGTCCACACCGGG | qPCR primer |
| LMP7 fwd | CATGGGCCATCTCAATCTG | qPCR primer |
| LMP7 rev | TCTCCAGAGCTCGCTTTACC | qPCR primer |
| HLA-A fwd | GCGGCTACTACAACCAGAGC | qPCR primer |
| HLA-A rev | GATGTAATCCTTGCCGTCGT | qPCR primer |
| HLA-B fwd | GACGGCAAGGATTACATCGCCCTGAA | qPCR primer |
| HLA-B rev | CACGGGCCGCCTCCCACT | qPCR primer |
| HLA-C fwd | GGAGACACAGAAGTACAAGCG | qPCR primer |
| HLA-C rev | CGTCGTAGGCGTACTGGTCATA | qPCR primer |
| HLA-E fwd | CCTACGACGGCAAGGA | qPCR primer |
| HLA-E rev | CCCTTCTCCAGGTATTTGTG | qPCR primer |
| NLRC5 fwd | CGCTCTGTGGCCACTTTCAG | qPCR primer |
| NLRC5 rev | TGCCCGCTGTGAGACTTCAT | qPCR primer |
| UBC fwd | ATTTGGGTCGCGGTTCTTG | qPCR primer |
| UBC rev | TGCCTTGACATTCTCGATGGT | qPCR primer |
| GAPDH fwd | TGCACCACCAACTGCTTAGC | qPCR primer |
| GAPDH rev | GGCATGGACTGTGGTCATGAG | qPCR primer |
| HPRT1 fwd | TGACACTGGCAAAACAATGCA | qPCR primer |
| HPRT1 rev | GGTCCTTTTCACCAGCAAGCT | qPCR primer |
| ACTB fwd | CTGGAACGGTGAAGGTGACA | qPCR primer |
| ACTB rev | AAGGGACTTCCTGTAACAATGCA | qPCR primer |

| | | |
|---|---|---|
| REAGENT or RESOURCE | SOURCE | IDENTIFIER |

Antibodies

| | | |
|---|---|---|
| Brilliant Violet 421-conjugated anti-human PD-L1 | Biolegend | Cat # 329714 |
| antibody | | |
| Brilliant Violet 421-conjugated anti-human CD56 | Biolegend | Cat # 318328 |
| antibody | | |
| Brilliant Violet 605-conjugated anti-human CD80 | Biolegend | Cat # 305225 |
| antibody | | |
| FITC-conjugated anti-human HLA-DR | Biolegend | Cat # 307604 |
| PE-conjugated anti-human OX40L antibody | Biolegend | Cat # 326308 |
| PE-conjugated anti-human HLA-E | Biolegend | Cat # 342604 |
| PerCP/Cy5.5-conjugated anti-human Galectin-9 | Biolegend | Cat # 348910 |
| antibody | | |
| 7AAD viability staining solution | Biolegend | Cat # 420403 |
| Human TruStain FcX | Biolegend | Cat # 422301 |
| PE-conjugated mouse IgG1 isotype control antibody | Biolegend | Cat # 400112 |
| Brilliant Violet 421-conjugated mouse IgG2b isotype | Biolegend | Cat # 400342 |
| control antibody | | |
| PerCP/Cy5.5-conjugated mouse IgG1 isotype control | Biolegend | Cat # 400149 |
| antibody | | |
| PE-conjugated mouse IgG1 isotype control antibody | Biolegend | Cat # 400111 |
| Brilliant Violet 605-conjugated anti-human CD47 | BD Biosciences | Cat # 563759 |
| antibody | | |
| Brilliant Violet 605-conjugated mouse IgG1 mouse | BD Biosciences | Cat # 562652 |
| isotype control antibody | | |
| FITC-conjugated mouse IgG2a isotype control antibody | BD Biosciences | Cat # 555573 |
| Alexa Fluor 647-conjugated anti-human MHC-I | Santa Cruz | Cat # sc32235 |
| antibody | Biotechnology | |
| Alexa Fluor 647 mouse IgG2a isotype control antibody | Santa Cruz | Cat # sc24637 |
| | Biotechnology | |

Chemicals, Peptides, and Recombinant Proteins

| | | |
|---|---|---|
| Heparin | Stem Cell | Cat # 07980 |
| | Technologies | |
| Human serum AB | Gemini Products | Cat # 100-318 |
| Cobimetinib | Selleckchem | Cat # S8041 |
| Pimasertib | Selleckchem | Cat # S1475 |
| Trametinib | Selleckchem | Cat # S2673 |
| Recombinant human IFN | Peprotech | Cat # 300-02BC |
| Recombinant human IFN | Peprotech | Cat # 300-02 |
| Recombinant human EGF | Miltenyi Biotec | Cat # 130-097- |
| | | 749 |
| Recombinant human FGF2 | Miltenyi Biotec | Cat# 130-093- |
| | | 564 |
| Recombinant human IL-2 | Amgen | N/A |
| Recombinant human IFN2b | R&D Systems | Cat # 1105-1 |
| β-mercaptoethanol | Gibco | Cat #21985023 |
| Sodium pyruvate | Gibco | Cat # 11360070 |
| HEPES | Gibco | Cat # 15630080 |
| Penicillin/streptomycin | Gibco | Cat # 15140122 |
| DMSO | Sigma Aldrich | Cat # D2650 |
| Hyaluronidase | Sigma Aldrich | Cat # H3506 |
| Collagenase I | Sigma Aldrich | Cat # C0130 |
| Tris | Sigma Aldrich | Cat # 252859 |
| EDTA | Sigma Aldrich | Cat # 03690 |
| Sodium chloride | Sigma Aldrich | Cat # 71376 |
| Triton-X | Sigma Aldrich | Cat # T9284 |
| Octyl β-d-glucopyranoside | Sigma Aldrich | Cat # 08001 |
| Phenylmethanesulfonyl fluoride | Sigma Aldrich | Cat # 78830 |
| Protease inhibitor cocktail | Sigma Aldrich | Cat # |
| | | 4693116001 |
| DNAse I | Sigma Aldrich | Cat # |
| | | 4716728001 |
| Gammabind Plus sepharose beads | GE Healthcare | Cat # 17-0886- |
| | | 01 |

Critical Commercial Assays

| | | |
|---|---|---|
| CellTrace CFSE Cell Proliferation Kit | Invitrogen | Cat # C34554 |
| EasySep Human NK Cell Isolation Kit | Stem Cell | Cat # 17955 |
| | Technologies | |
| Live/Dead Fixable Aqua Dead Cell Stain Kit | ThermoFisher | Cat # L34965 |
| Venor ™ GeM Mycoplasma Detection Kit | Sigma Aldrich | Cat # MP0025 |

Software and Algorithms

| | | |
|---|---|---|
| FlowJo | TreeStar | N/A |

### Example 2: MCC Cell Lines are Reliably Generated from Primary Patient Samples

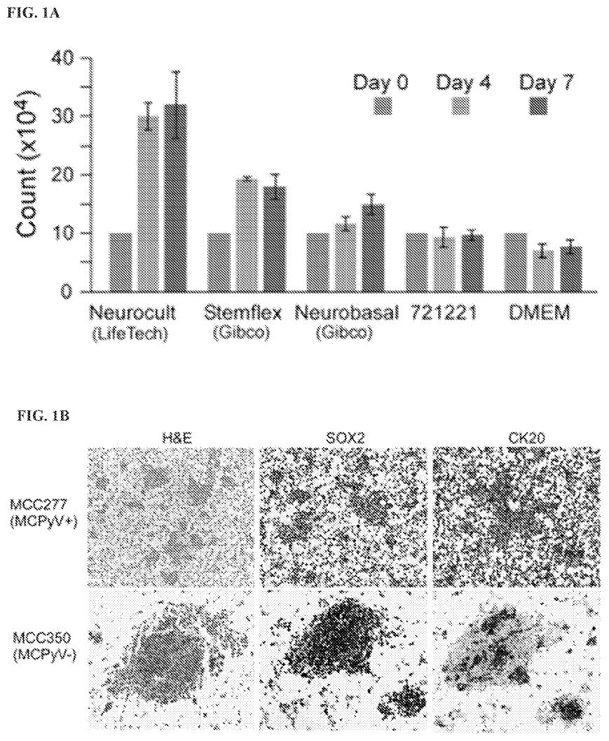

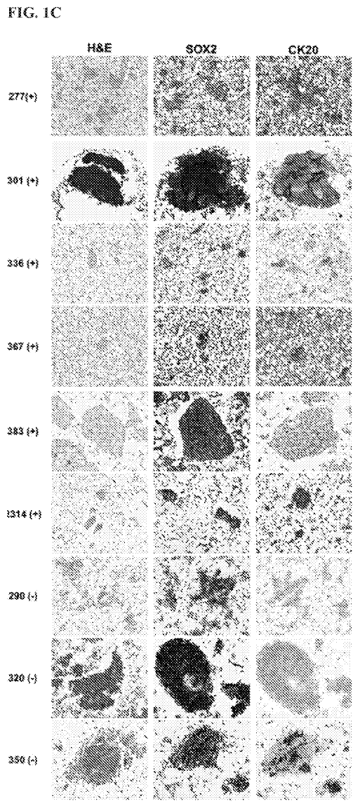

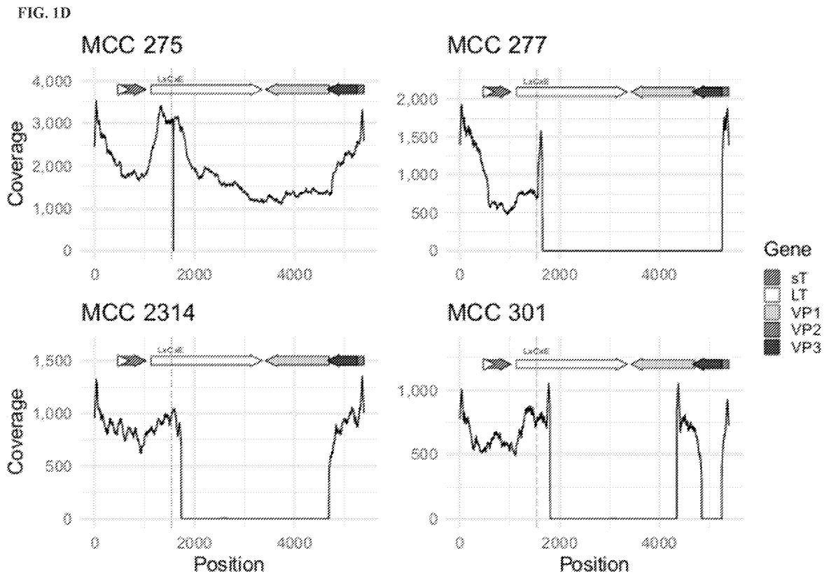

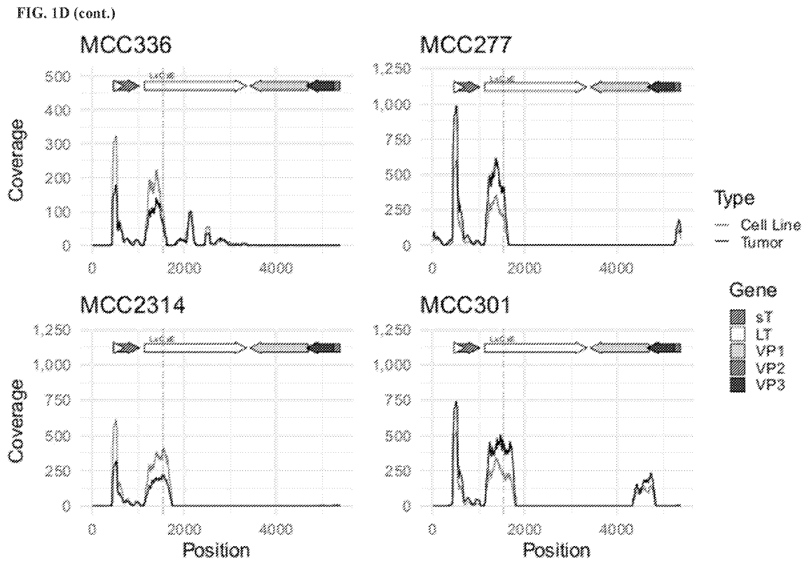

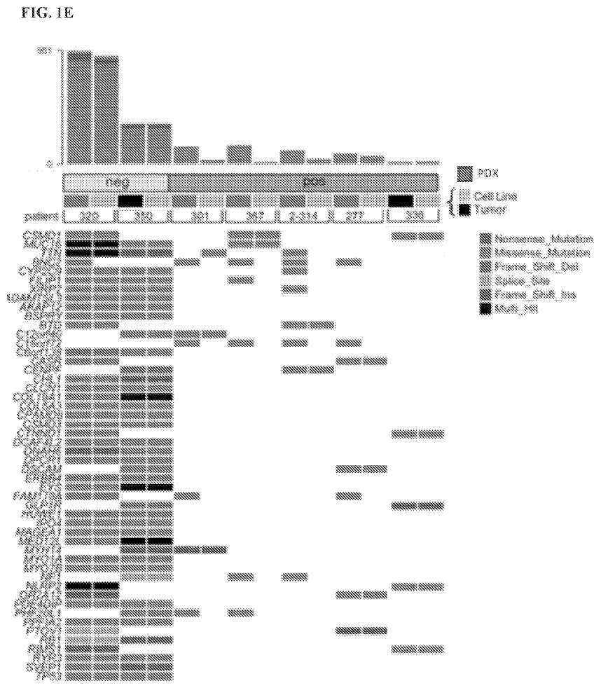

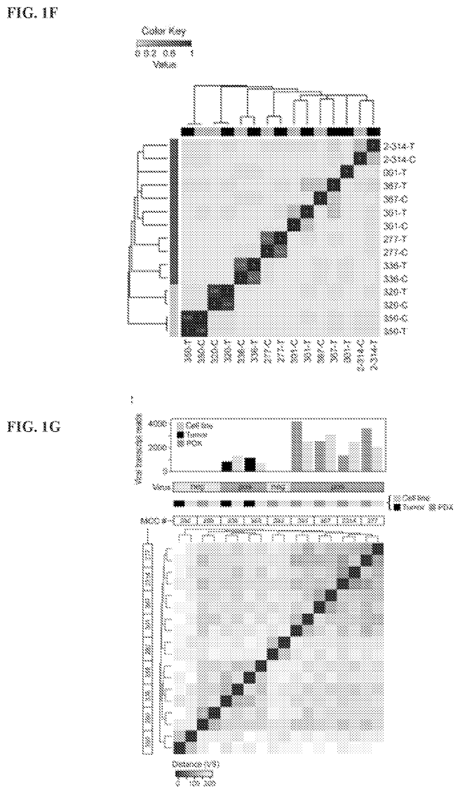

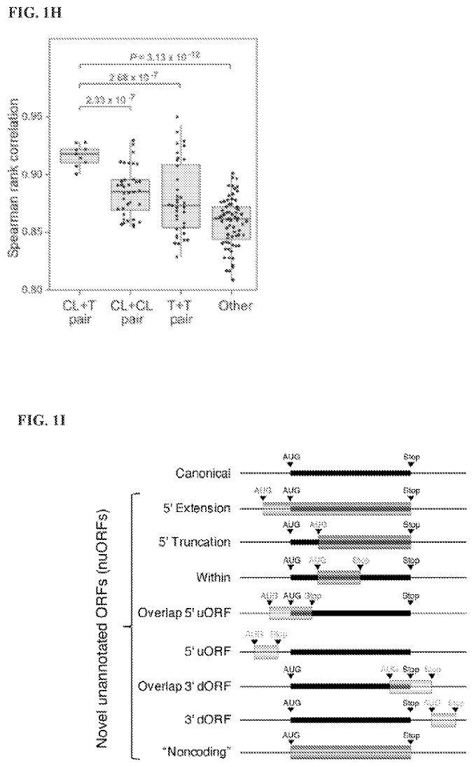

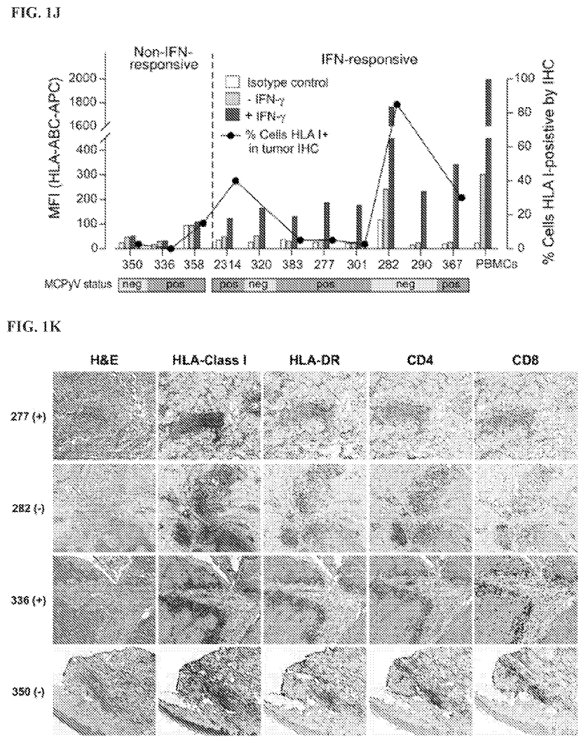

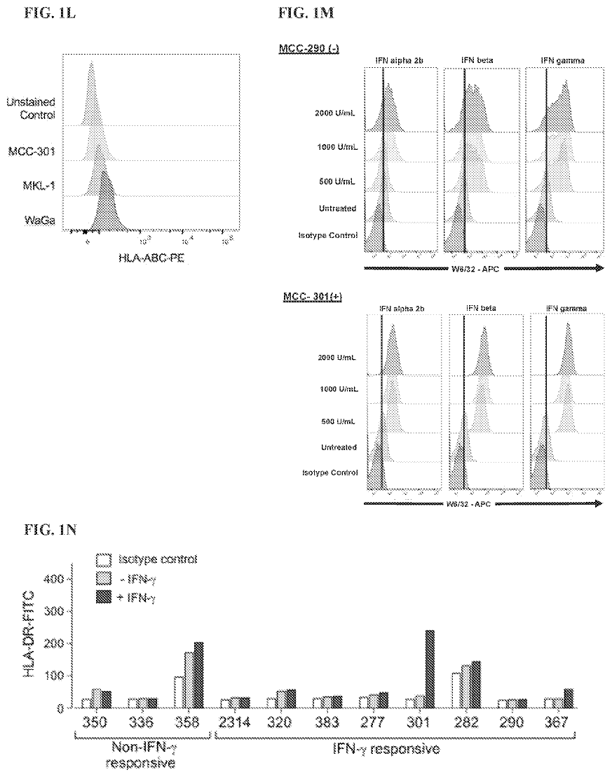

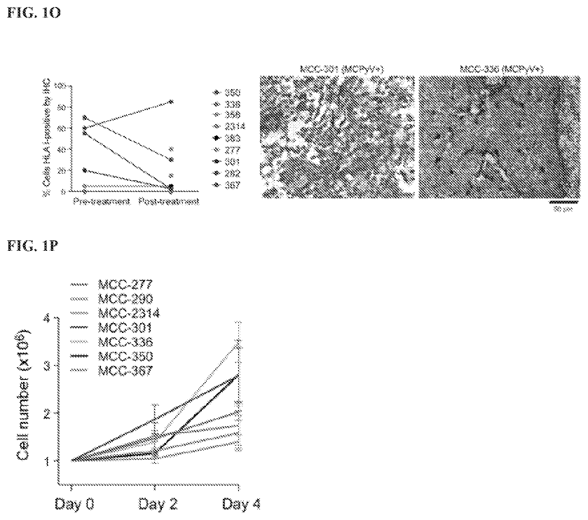

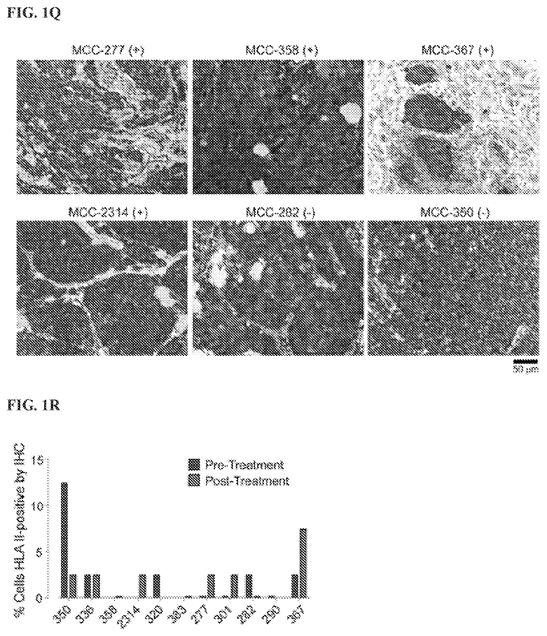

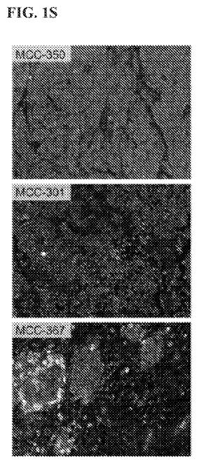

Many established MCC lines, typically cultured in an RPMI-1640 based media formulation have been multiply passaged in vitro and commonly lack associated archival primary tumor material and clinical data. To establish a series of lines directly from patient specimens, conditions were optimized to generate a reliable approach to propagate MCC cell lines in vitro. Since MCC tumors exhibit neuroendocrine histology and another panel of MCC lines had been successfully established in a modified neural crest stem cell medium, it was hypothesized that culturing these cell lines in a neuronal stem cell media that was previously used to establish glioblastoma multiforme tumor cell lines would facilitate cell line establishment. Five media formulations were tested on the MCC-336 tumor specimen, and Neurocult NS-A Proliferation medium with growth factor supplementation consistently provided the highest in vitro growth rate, tripling cell numbers after seven days in culture (FIG. 1A).

Using this method, a total of 11 stable cell lines were established from biopsies (n=4) or patient-derived xenograft (PDX) materials (n=7) (Table 6). Consistent with previously established MCC lines, these cell lines were observed to grow mostly in tight clusters in suspension and stained positive for CK20 and SOX2, classical immunohistochemical markers of MCC (FIGS. 1B, 1C). Using a Viro-Panel, a hybrid-capture based sequencing platform, to detect 447 cancer related genes and 19 oncoviruses, 7 of 11 lines were positive for MCPyV, while 4 were MCPyV⁻ (FIG. 1D).

TABLE 6

| | | Cell | MCPyV | | | History of |
|---|---|---|---|---|---|---|
| | | Line | Viral | | Response to | immune |
| Patient | Gender | Source | Status | Prior Treatment | PD1:PD-L1 | suppression |
| 277 | M | PDX | Virus-positive | CE; | CR | — |
| | | | | chemoradiation; | | |
| | | | | MLN0128; CAV; | | |
| | | | | octreotide; | | |
| | | | | imiquimod; | | |
| | | | | cabozantinib | | |
| 282 | M | PDX | Virus-negative | XRT | — | heart transplant |
| 290 | F | PDX | Virus-negative | — | — | — |
| 301 | M | PDX | Virus-positive | CE, | PD | — |
| | | | | chemoradiation | | |
| 320 | M | PDX | Virus-negative | CE; | — | — |
| | | | | chemoradiation | | |
| 336 | F | Tumor | Virus-positive | CE, | — | — |
| | | | | chemoradiation | | |
| 350 | M | Tumor | Virus-negative | XRT | PD | — |
| 358 | F | Tumor | Virus-positive | XRT | Discontinued due to | rheumatoid |
| | | | | | side effects | arthritis on |
| | | | | | | adalimumab |
| 367 | M | PDX | Virus-positive | XRT | — | — |
| 383 | M | Tumor | Virus-positive | XRT | adjuvant | — |
| 2314 | F | PDX | Virus-positive | Everolimus; CE; | — | — |
| | | | | Paclitaxel | | |

For 7 of 11 patients, matched peripheral blood mononuclear cells (PBMCs) were available from which germline DNA was extracted. Whole-exome sequencing (WES) of DNA from matched primary tumor, cell line, and germline source was performed for these lines, as well as RNA-sequencing (RNAseq). These studies revealed the cell lines that display genetic alterations characteristic of MCC, as well as genomic and transcriptional similarity between corresponding tumor and cell lines (Table 6). MCPyV⁻ and MCPyV⁺ samples exhibited the expected contrasting high (median 647 non-silent coding mutations per cell line, range 354-940) and low (median 40, range 18-73) mutational burdens (FIG. 1E and data not shown), respectively. Moreover, the two analyzed MCPyV− lines both contained mutations in RBI and TP53 (data not shown), consistent with previous studies (Goh et al. (2016) Oncotarget 7 (3): 3403-15); Knepper et al. (2019a) Clinical Cancer Research 25 (19): 5961-71). Of the mutations found in each cell line, a median of 94.4% were also detected in corresponding tumor or PDX samples (range 51-100%), and tumor-cell line pairs associated most closely with each other based on mutational profiles (FIG. 1F). Of note, several PDX-derived tumor samples (Table 6) exhibited higher mutational burdens than their corresponding cell lines (FIG. 1E), likely due to murine cell contamination.

Within the RNAseq data, transcripts mapping to the MCPyV ST and LT antigens were data detected in all samples determined to be MCPyV+ by ViroPanel (FIG. 1D, 1G). By unsupervised hierarchical clustering analysis of MCC tumors and cell lines based on RNA-seq, each cell line was observed to associate most closely with its corresponding parent tumor (mean pairwise Spearman correlation 0.92) (FIG. 1H), rather than to cluster by sample type, confirming that these cell lines faithfully recapitulate the tumors from which they were derived. Additionally, Ribo-seq can be used to predict translated unannotated ORFs (FIG. 1I)

In addition to the consistency in the genetic and transcriptional profiles of the generated cell lines in relation to parental tumors, the lines also displayed consistent defects in surface HLA I surface expression like their parental tumors. By flow cytometry using a pan-class I anti-HLA-ABC antibody, all 11 lines strikingly exhibited low, nearly absent HLA I (FIG. 1J). Such absence of in situ HLA class I expression on MCC cells was confirmed by immunohistochemical staining of the parental tumors for 4 lines (FIG. 1K). Moreover, the low class I surface expression by flow cytometry was on par with well-studied MCPyV+ lines MKL-1 and WaGa (FIG. 1L). Three lines were not responsive to IFN-y exposure (MCC-336, -350, -358), whereas 8 lines exhibited HLA I surface expression that could be induced by IFN-y (median 5.7-fold increase by MFI, range 2.5-12.4). For two lines, HLA I could be upregulated by IFN-a-2b and IFN-13 (FIG. 1M) and another line (MCC-301) had inducible HLA-DR expression with IFN-y as well (FIG. 1N). These data suggest that the majority of MCC samples have reversible HLA class I pathway defects at the transcriptional rather than at the genomic level.

### Example 3: MCC Lines Exhibit Transcriptional Downregulation of Multiple Class I Genes with Underlying NLRC5 Alterations

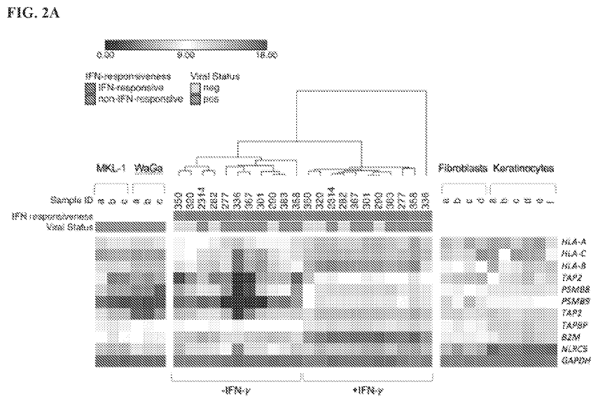

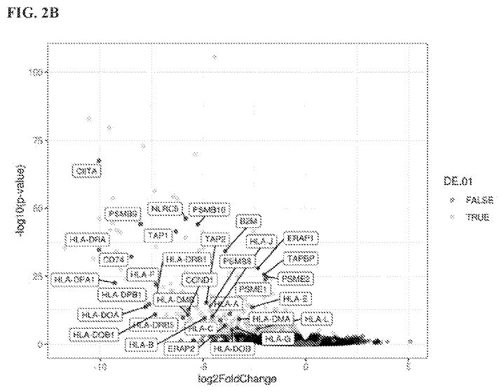

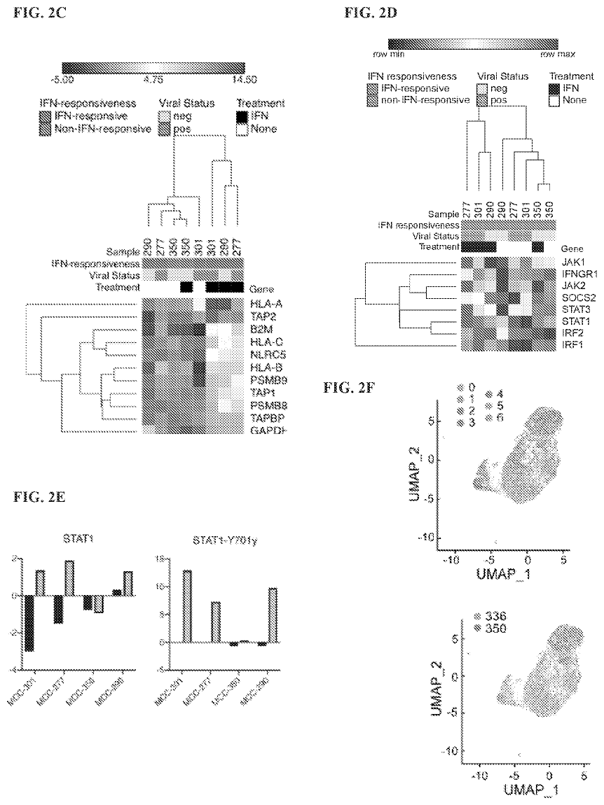

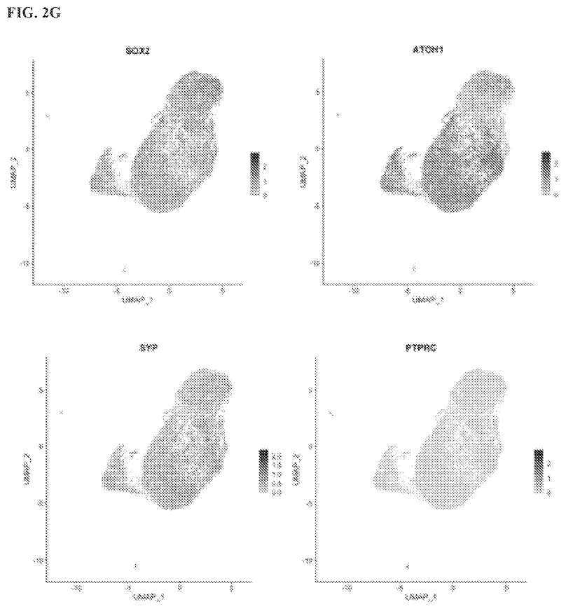

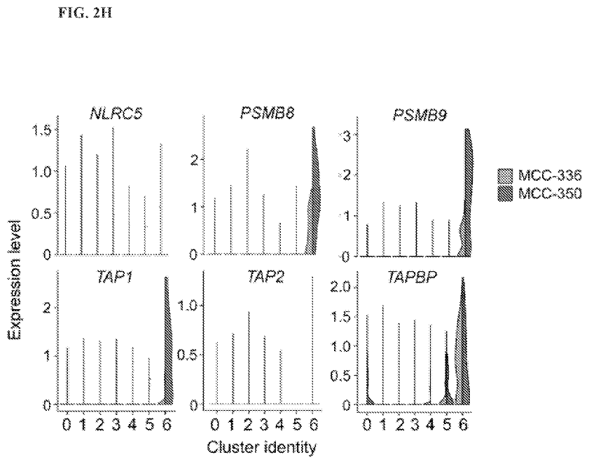

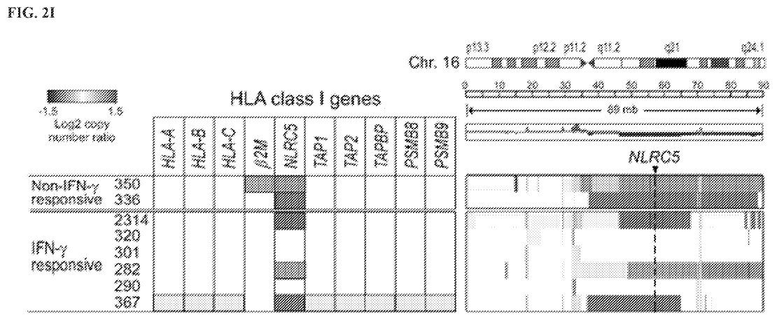

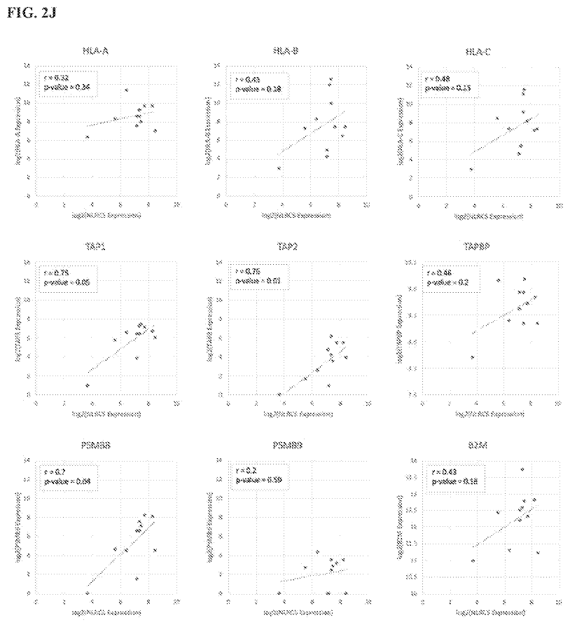

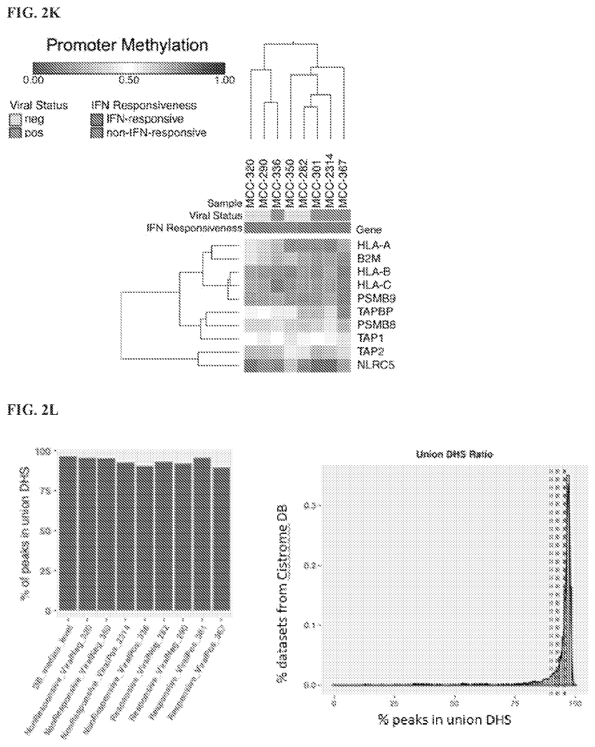

To elucidate the mechanisms of impaired HLA I surface expression in the MCC lines, in-depth genomic and epigenomic characterizations were performed for a subset of both virus-positive and -negative lines for which material was available (Table 7). To define the alterations in gene expression in MCC after IFN-y exposure, transcript expression was evaluated in all 11 MCC lines at baseline and after IFN-y stimulation. The expression of the MCC lines was compared to epidermal keratinocytes and dermal fibroblasts (Butterfield et al. (2017) PloS One 12 (12): e0189664); Swindell et al. (2017) Scientific Reports 7 (1): 18045), since they are leading candidates for the cell-of-origin of MCPyV− and MCPyV+ MCC, respectively (Sunshine et al. (2018) Oncogene 37 (11): 1409-16), and both reside within the skin. At baseline, the MCC lines exhibited low mRNA expression of several class I pathway genes, most notably HLA-B, TAP1, TAP2, PSMB8, and PSMB9, with a generally similar expression profile in MKL-1 and WaGa, two well-studied MCC lines (FIG. 2A). IFN-y treatment markedly upregulated class I genes in 10 of 11 MCC lines (FIG. 2B; Table 8), a trend which was confirmed in matched proteomes in 4 MCC lines (FIG. 2C). MCC lines that were non-IFN-responsive by flow cytometry (FIG. 1I) exhibited variable defects, such as lack of IFN-induced HLA-A, -B, and -C mRNA upregulation (MCC-336) and lack of IFN-induced STAT1/p-STAT1 protein expression (FIGS. 2A, 2D), 2E).

TABLE 7

| | Tumor | | | Cell | | | | | |
|---|---|---|---|---|---|---|---|---|---|
| | and | | | Line +/− | Cell Line +/− | | | | |
| | Cell | Cell | Tumor | IFN: | IFN: Full and | | | Tumor: | Cell Line +/− |
| | Line | Line | RNA- | RNA- | Phospho- | | | HLA | IFN: HLA |
| Patient | WES | WGS | seq | seq | Proteome | ATAC-seq | WGBS | Peptidome | Peptidome |
| 277 | X | X | X | X | X | X | X | X | X |
| 282 | | X | X | X | | X | X | | |
| 290 | | X | X | X | X | X | X | X | X |
| 301 | X | X | X | X | X | X | X | | X |
| 320 | X | X | | X | | X | X | | |
| 336 | X | X | X | X | | X | X | | |
| 350 | X | X | X | X | X | X | X | | |
| 358 | | | X | X | | | | | |
| 367 | X | X | X | X | | X | X | | X |
| 383 | | | | X | | | | | |
| 2314 | X | X | X | X | | X | X | | |

TABLE 8
DESeq Analysis of 11 MCC cell lines at baseline and after IFNγ stimulation. FDR
0.01 cutoff Negative LFC indicates upregulation in the IFNγ-treated samples

| baseMean | log2FoldChange | lfcSE | stat | pvalue | padj | ensemblID | symbol |
|---|---|---|---|---|---|---|---|
| 737.61219 | −3.8235466 | 0.49197449 | −7.7718391 | 7.74E−15 | 2.30E−12 | ENSG00000187608.5 | ISG15 |
| 429.856494 | −3.995204 | 0.60395352 | −6.6150853 | 3.71E−11 | 7.96E−09 | ENSG00000157873.13 | TNFRSF14 |
| 2.53224988 | −4.2047423 | 0.94141369 | −4.466413 | 7.95E−06 | 0.00090579 | ENSG00000225931.3 | NA |
| 306.683679 | −2.8543837 | 0.36225466 | −7.8794946 | 3.29E−15 | 1.03E−12 | ENSG00000116663.6 | FBXO6 |
| 30.9609199 | −3.2331262 | 0.75273663 | −4.2951625 | 1.75E−05 | 0.00193537 | ENSG00000173369.11 | C1QB |
| 4505.34673 | −2.3277354 | 0.45338652 | −5.1341081 | 2.83E−07 | 3.82E−05 | ENSG00000126709.10 | IFI6 |
| 1565.32413 | −0.5387475 | 0.13041279 | −4.1310938 | 3.61E−05 | 0.00363267 | ENSG00000220785.3 | MTMR9LP |
| 3432.58269 | −0.8512104 | 0.20898338 | −4.0731007 | 4.64E−05 | 0.00455845 | ENSG00000116514.12 | RNF19B |
| 807.146321 | −1.2235072 | 0.28307901 | −4.3221404 | 1.55E−05 | 0.00172619 | ENSG00000142920.12 | AZIN2 |
| 4668.38365 | 0.67628768 | 0.14788691 | 4.57300581 | 4.81E−06 | 0.00055831 | ENSG00000117399.9 | CDC20 |
| 733.073398 | −1.493707 | 0.27795021 | −5.3740092 | 7.70E−08 | 1.13E−05 | ENSG00000142961.10 | MOB3C |
| 5.45481892 | −5.1726162 | 0.89251196 | −5.7955707 | 6.81E−09 | 1.12E−06 | ENSG00000230812.1 | NA |
| 8603.43775 | −0.3836358 | 0.09904608 | −3.8733064 | 0.00010737 | 0.00969881 | ENSG00000077254.10 | USP33 |
| 130.523058 | −7.7332581 | 0.96504001 | −8.0134068 | 1.12E−15 | 3.68E−13 | ENSG00000137959.11 | IFI44L |
| 887.55803 | −8.9543704 | 1.01498021 | −8.8222119 | 1.12E−18 | 4.52E−16 | ENSG00000137965.6 | IFI44 |
| 50.0400965 | −1.8278699 | 0.31760936 | −5.7550883 | 8.66E−09 | 1.42E−06 | ENSG00000122432.12 | SPATA1 |
| 186.636266 | −9.2148342 | 0.67374151 | −13.677106 | 1.39E−42 | 2.27E−39 | ENSG00000117226.7 | GBP3 |
| 10681.5088 | −10.095742 | 0.81082348 | −12.45122 | 1.38E−35 | 1.40E−32 | ENSG00000117228.9 | GBP1 |
| 284.534786 | −6.2350364 | 0.72993711 | −8.5418817 | 1.32E−17 | 4.91E−15 | ENSG00000162645.8 | GBP2 |
| 758.052751 | −10.330761 | 0.87528061 | −11.802799 | 3.78E−32 | 3.08E−29 | ENSG00000162654.8 | GBP4 |
| 276.049095 | −5.8538857 | 0.44265315 | −13.224543 | 6.33E−40 | 7.75E−37 | ENSG00000154451.10 | GBP5 |
| 3588.44644 | −7.9265386 | 0.43681701 | −18.146131 | 1.38E−73 | 1.01E−69 | ENSG00000225492.2 | GBP1P1 |
| 6252.06883 | −0.7264747 | 0.13566003 | −5.3551124 | 8.55E−08 | 1.22E−05 | ENSG00000143106.8 | PSMA5 |
| 765.074779 | −3.7414089 | 0.36225487 | −10.328112 | 5.26E−25 | 2.97E−22 | ENSG00000184371.9 | CSF1 |
| 5881.08817 | −1.6522279 | 0.24625693 | −6.7093662 | 1.95E−11 | 4.29E−09 | ENSG00000155363.14 | MOV10 |
| 3778.53797 | −0.3499462 | 0.08801924 | −3.9757917 | 7.01E−05 | 0.00671295 | ENSG00000121848.9 | NA |
| 32585.8155 | −1.0676432 | 0.18111126 | −5.8949576 | 3.75E−09 | 6.36E−07 | ENSG00000160710.11 | ADAR |
| 163.260719 | −5.9462553 | 1.233604 | −4.8202303 | 1.43E−06 | 0.00017851 | ENSG00000163565.14 | IFI16 |
| 1241.49316 | −0.6380783 | 0.13586087 | −4.6965572 | 2.65E−06 | 0.000319 | ENSG00000000457.9 | SCYL3 |
| 278.174332 | −3.2156169 | 0.50940837 | −6.312454 | 2.75E−10 | 5.45E−08 | ENSG00000235750.5 | KIAA0040 |
| 604.684858 | −3.4654178 | 0.7512412 | −4.6129229 | 3.97E−06 | 0.00046669 | ENSG00000184731.5 | FAM110C |
| 27.9091113 | −3.7242927 | 0.75334044 | −4.9437048 | 7.67E−07 | 9.83E−05 | ENSG00000225964.1 | NRIR |
| 1011.67564 | −2.8295832 | 0.45739818 | −6.1862581 | 6.16E−10 | 1.17E−07 | ENSG00000134326.7 | CMPK2 |
| 1525.78994 | −4.6385891 | 0.52492551 | −8.8366616 | 9.86E−19 | 4.02E−16 | ENSG00000134321.7 | RSAD2 |
| 154.723863 | −0.8530222 | 0.20559557 | −4.14903 | 3.34E−05 | 0.00339433 | ENSG00000173567.10 | ADGRF3 |
| 22.519641 | −3.8039207 | 0.49760753 | −7.6444194 | 2.10E−14 | 5.87E−12 | ENSG00000152689.13 | RASGRP3 |
| 9628.05326 | −1.3672396 | 0.24566348 | −5.5654979 | 2.61E−08 | 4.04E−06 | ENSG00000055332.12 | EIF2AK2 |
| 317.694297 | −0.5688074 | 0.13836195 | −4.1110102 | 3.94E−05 | 0.00395007 | ENSG00000144182.12 | LIPT1 |
| 19.3370989 | −2.3383337 | 0.47489185 | −4.9239289 | 8.48E−07 | 0.00010788 | ENSG00000224789.1 | NA |
| 1642.86004 | −1.1988465 | 0.29247544 | −4.0989646 | 4.15E−05 | 0.00413314 | ENSG00000144118.9 | RALB |
| 47.5933423 | −2.8699117 | 0.51644397 | −5.557063 | 2.74E−08 | 4.22E−06 | ENSG00000054219.9 | LY75 |
| 2144.22498 | −4.034425 | 0.51604644 | −7.8179495 | 5.37E−15 | 1.63E−12 | ENSG00000115267.5 | IFIH1 |
| 2863.88892 | −0.5343727 | 0.12805713 | −4.1729246 | 3.01E−05 | 0.00308916 | ENSG00000138433.11 | CIR1 |
| 2998.87025 | −1.931128 | 0.45637728 | −4.2314288 | 2.32E−05 | 0.00246295 | ENSG00000151689.8 | INPPI |
| 62446.3512 | −4.434093 | 0.20252029 | −21.894561 | 2.93E−106 | 8.60E−102 | ENSG00000115415.14 | STAT1 |
| 15.6568451 | −4.6685599 | 0.56687721 | −8.2355753 | 1.79E−16 | 6.10E−14 | ENSG00000229023.1 | NA |
| 358.983013 | −1.0983337 | 0.24195208 | −4.5394677 | 5.64E−06 | 0.00064724 | ENSG00000163251.3 | FZD5 |
| 532.489006 | −4.1539852 | 0.71671084 | −5.7959012 | 6.80E−09 | 1.12E−06 | ENSG00000188282.8 | RUFY4 |
| 2633.99179 | −1.70319 | 0.16583086 | −10.270646 | 9.56E−25 | 5.30E−22 | ENSG00000123992.14 | DNPEP |
| 822.116878 | −5.9124606 | 0.78305481 | −7.5505068 | 4.34E−14 | 1.17E−11 | ENSG00000135899.12 | SP110 |
| 4.38787916 | −4.0713127 | 0.75815716 | −5.3700115 | 7.87E−08 | 1.15E−05 | ENSG00000079263.14 | SP140 |
| 31.9574927 | −4.748526 | 0.92110029 | −5.1552757 | 2.53E−07 | 3.44E−05 | ENSG00000185404.12 | SP140L |
| 176.899362 | −6.1781587 | 0.74044969 | −8.3437927 | 7.19E−17 | 2.55E−14 | ENSG00000067066.12 | SP100 |
| 489.003471 | −1.1875496 | 0.29529724 | −4.0215398 | 5.78E−05 | 0.00557713 | ENSG00000163702.14 | IL17RC |
| 3.90267133 | −5.1220192 | 1.0270859 | −4.9869433 | 6.13E−07 | 7.94E−05 | ENSG00000231280.1 | NA |
| 2641.45613 | −5.0035905 | 0.36904215 | −13.558317 | 7.07E−42 | 9.45E−39 | ENSG00000182179.6 | UBA7 |
| 913.021769 | −1.7258766 | 0.36940042 | −4.6721025 | 2.98E−06 | 0.00035606 | ENSG00000041880.10 | PARP3 |
| 7336.92706 | −5.1123294 | 0.44475489 | −11.494712 | 1.40E−30 | 1.06E−27 | ENSG00000138496.12 | PARP9 |
| 5785.99283 | −4.6029819 | 0.45433708 | −10.131205 | 4.02E−24 | 2.07E−21 | ENSG00000163840.5 | DTX3L |
| 1.86850191 | −3.777983 | 0.93951153 | −4.0212205 | 5.79E−05 | 0.00557713 | ENSG00000173200.8 | PARP15 |
| 12051.8632 | −9.5124504 | 0.69528453 | −13.681378 | 1.31E−42 | 2.27E−39 | ENSG00000173193.9 | PARP14 |
| 16636.4037 | −0.5501989 | 0.1367503 | −4.0233836 | 5.74E−05 | 0.00556261 | ENSG00000017260.15 | ATP2C1 |
| 39.9014474 | −3.6489879 | 0.66551795 | −5.4829293 | 4.18E−08 | 6.30E−06 | ENSG00000251011.1 | TMEM108-AS1 |
| 2488.92758 | −1.9175703 | 0.39534607 | −4.8503589 | 1.23E−06 | 0.00015407 | ENSG00000188313.8 | PLSCR1 |
| 86.4459157 | −3.1923753 | 0.7519567 | −4.2454245 | 2.18E−05 | 0.00233946 | ENSG00000114805.12 | PLCH1 |
| 66.8300456 | −4.1315141 | 0.77211564 | −5.3509007 | 8.75E−08 | 1.24E−05 | ENSG00000114204.10 | SERPINI2 |
| 162.237393 | −4.563601 | 0.73662697 | −6.1952673 | 5.82E−10 | 1.11E−07 | ENSG00000174776.6 | WDR49 |
| 15.5030652 | −4.4304635 | 0.88651062 | −4.9976429 | 5.80E−07 | 7.54E−05 | ENSG00000121858.6 | TNFSF10 |
| 100.27005 | −9.7664391 | 0.83826389 | −11.650793 | 2.27E−31 | 1.81E−28 | ENSG00000136514.2 | RTP4 |
| 322.630799 | −2.9529352 | 0.52640004 | −5.609679 | 2.03E−08 | 3.15E−06 | ENSG00000113916.13 | BCL6 |
| 6892.05182 | −1.8713028 | 0.19948294 | −9.380766 | 6.55E−21 | 2.96E−18 | ENSG00000002549.8 | LAP3 |
| 211.193901 | −2.6467285 | 0.46907387 | −5.6424556 | 1.68E−08 | 2.66E−06 | ENSG00000185774.10 | KCNIP4 |
| 1743.57366 | −2.41664 | 0.56954916 | −4.2430754 | 2.20E−05 | 0.00235276 | ENSG00000145246.9 | ATP10D |
| 357.247212 | −7.1736796 | 0.90792832 | −7.9011519 | 2.76E−15 | 8.73E−13 | ENSG00000128052.8 | KDR |
| 29.8302512 | −1.9353111 | 0.42562106 | −4.5470284 | 5.44E−06 | 0.00062687 | ENSG00000249700.4 | SRD5A3-AS1 |
| 613.01113 | −11.741625 | 0.93673037 | −12.53469 | 4.82E−36 | 5.25E−33 | ENSG00000138755.5 | CXCL9 |
| 463.70535 | −5.8896056 | 0.52056523 | −11.313867 | 1.12E−29 | 8.03E−27 | ENSG00000169245.4 | CXCL10 |
| 400.13107 | −4.6722976 | 0.64941778 | −7.1945945 | 6.26E−13 | 1.56E−10 | ENSG00000169248.8 | CXCL11 |
| 971.948802 | −9.026515 | 0.81940486 | −11.01594 | 3.20E−28 | 2.19E−25 | ENSG00000138642.10 | HERC6 |
| 826.4622 | −2.1560071 | 0.55713086 | −3.8698397 | 0.00010891 | 0.00978497 | ENSG00000138646.4 | HERC5 |
| 27.3654855 | −6.7810866 | 1.00625711 | −6.7389204 | 1.60E−11 | 3.52E−09 | ENSG00000164136.12 | IL15 |
| 10.6710715 | −4.8732259 | 0.94650465 | −5.148655 | 2.62E−07 | 3.55E−05 | ENSG00000183090.5 | FREM3 |
| 540.179187 | −3.0051706 | 0.4150247 | −7.2409438 | 4.46E−13 | 1.12E−10 | ENSG00000256043.2 | CTSO |
| 4007.1413 | −7.2981902 | 0.57962796 | −12.591163 | 2.36E−36 | 2.67E−33 | ENSG00000137628.12 | DDX60 |
| 1279.85024 | −3.4606835 | 0.43152536 | −8.0196526 | 1.06E−15 | 3.54E−13 | ENSG00000181381.9 | DDX60L |
| 1441.65616 | −1.5881183 | 0.17833775 | −8.9051157 | 5.33E−19 | 2.21E−16 | ENSG00000168310.6 | IRF2 |
| 123.515844 | −1.8533896 | 0.30669268 | −6.0431492 | 1.51E−09 | 2.72E−07 | ENSG00000271646.1 | NA |
| 16.9034358 | −6.0189651 | 0.88029443 | −6.8374455 | 8.06E−12 | 1.87E−09 | ENSG00000164342.8 | TLR3 |
| 46.0643916 | −3.64788 | 0.51514091 | −7.0813246 | 1.43E−12 | 3.50E−10 | ENSG00000248693.1 | NA |
| 3950.73396 | −2.3307071 | 0.20970864 | −11.114025 | 1.07E−28 | 7.50E−26 | ENSG00000164307.8 | ERAP1 |
| 2357.23316 | −3.9313244 | 0.74683526 | −5.2639781 | 1.41E−07 | 1.99E−05 | ENSG00000164308.12 | ERAP2 |
| 143.593351 | −1.8949887 | 0.36326139 | −5.2165983 | 1.82E−07 | 2.54E−05 | ENSG00000238000.1 | NA |
| 1231.90737 | −3.7099074 | 0.34488155 | −10.757048 | 5.49E−27 | 3.58E−24 | ENSG00000197536.6 | C5orf56 |
| 13.6015475 | −3.2740327 | 0.63313427 | −5.1711506 | 2.33E−07 | 3.19E−05 | ENSG00000238160.1 | NA |
| 45.7490278 | −3.6838099 | 0.47064419 | −7.8271652 | 4.99E−15 | 1.53E−12 | ENSG00000202533.1 | NA |
| 43.5846283 | −2.2803371 | 0.38251438 | −5.9614416 | 2.50E−09 | 4.37E−07 | ENSG00000234290.2 | NA |
| 8827.1479 | −5.3847842 | 0.30310638 | −17.765328 | 1.31E−70 | 7.71E−67 | ENSG00000125347.9 | IRF1 |
| 8506.34864 | −8.4943849 | 0.71110038 | −11.945409 | 6.86E−33 | 5.76E−30 | ENSG00000019582.10 | CD74 |
| 42.2431901 | −4.6559605 | 0.76758996 | −6.0656871 | 1.31E−09 | 2.38E−07 | ENSG00000113263.8 | ITK |
| 2403.05295 | −6.0891068 | 0.78439158 | −7.7628406 | 8.30E−15 | 2.44E−12 | ENSG00000186470.9 | BTN3A2 |
| 2750.38209 | −6.5816686 | 0.66694197 | −9.8684277 | 5.71E−23 | 2.79E−20 | ENSG00000026950.12 | BTN3A1 |
| 27.1509251 | −4.4586863 | 1.01379967 | −4.3979954 | 1.09E−05 | 0.00122516 | ENSG00000124549.10 | BTN2A3P |
| 2209.06227 | −6.9296874 | 0.72527379 | −9.5545812 | 1.24E−21 | 5.79E−19 | ENSG00000111801.11 | BTN3A3 |
| 1248.67163 | −0.9872832 | 0.25467903 | −3.8765782 | 0.00010594 | 0.00960615 | ENSG00000112763.11 | BTN2A1 |
| 2104.13959 | −7.2893033 | 0.74532007 | −9.7800979 | 1.37E−22 | 6.50E−20 | ENSG00000204642.9 | HLA-F |
| 6.65168958 | −3.3667593 | 0.68997805 | −4.8795166 | 1.06E−06 | 0.00013352 | ENSG00000204632.7 | HLA-G |
| 594.384724 | −3.8830179 | 0.62100295 | −6.2528171 | 4.03E−10 | 7.90E−08 | ENSG00000206341.6 | HLA-H |
| 12183.3964 | −3.7023024 | 0.53944569 | −6.8631606 | 6.74E−12 | 1.58E−09 | ENSG00000206503.7 | HLA-A |
| 27.7431104 | −4.551376 | 0.69817858 | −6.5189282 | 7.08E−11 | 1.47E−08 | ENSG00000204622.6 | HLA-J |
| 4015.70697 | −0.6803101 | 0.11881275 | −5.7259015 | 1.03E−08 | 1.66E−06 | ENSG00000234127.4 | TRIM26 |
| 28.825266 | −1.2459717 | 0.25318923 | −4.9211086 | 8.61E−07 | 0.00010898 | ENSG00000233892.1 | NA |
| 63.6973362 | −2.336374 | 0.49375517 | −4.7318473 | 2.22E−06 | 0.00027123 | ENSG00000243753.1 | HLA-L |
| 29328.8204 | −2.6080529 | 0.34230442 | −7.6191037 | 2.55E−14 | 7.01E−12 | ENSG00000204592.5 | HLA-E |
| 15690.8228 | −4.1551201 | 0.68961204 | −6.025301 | 1.69E−09 | 3.01E−07 | ENSG00000204525.10 | HLA-C |
| 26351.7068 | −4.9211669 | 0.77622894 | −6.3398395 | 2.30E−10 | 4.60E−08 | ENSG00000234745.5 | HLA-B |
| 2.65118174 | −4.1377374 | 0.87127079 | −4.7490832 | 2.04E−06 | 0.00025119 | ENSG00000271581.1 | NA |
| 1709.10902 | −5.131482 | 0.73627891 | −6.9694812 | 3.18E−12 | 7.60E−10 | ENSG00000206337.6 | HCP5 |
| 286.616839 | −4.0960936 | 0.49414563 | −8.2892437 | 1.14E−16 | 3.94E−14 | ENSG00000166278.10 | C2 |
| 87.8474596 | −7.332932 | 0.73464244 | −9.9816341 | 1.83E−23 | 9.29E−21 | ENSG00000243649.4 | CFB |
| 190.73699 | −1.2449859 | 0.22451401 | −5.5452481 | 2.94E−08 | 4.47E−06 | ENSG00000244731.3 | C4A |
| 135.903291 | −2.2481543 | 0.419693 | −5.3566639 | 8.48E−08 | 1.21E−05 | ENSG00000224389.4 | C4B |
| 2051.81717 | −10.073772 | 0.81091412 | −12.422736 | 1.97E−35 | 1.93E−32 | ENSG00000204287.9 | HLA-DRA |
| 9.8285994 | −5.9804428 | 0.93728232 | −6.3806205 | 1.76E−10 | 3.60E−08 | ENSG00000198502.5 | HLA-DRB5 |
| 110.723979 | −7.2780908 | 0.84423139 | −8.6209669 | 6.64E−18 | 2.53E−15 | ENSG00000196126.6 | HLA-DRB1 |
| 151.99633 | −7.3348967 | 1.07983707 | −6.7925958 | 1.10E−11 | 2.51E−09 | ENSG00000179344.12 | HLA-DQB1 |
| 187.06255 | −3.5979372 | 0.78128091 | −4.6051774 | 4.12E−06 | 0.00048239 | ENSG00000241106.2 | HLA-DOB |
| 5778.85898 | −4.8445298 | 0.59768989 | −8.1054238 | 5.26E−16 | 1.78E−13 | ENSG00000204267.9 | TAP2 |
| 4981.82102 | −4.6241388 | 0.59776915 | −7.7356599 | 1.03E−14 | 2.99E−12 | ENSG00000204264.4 | PSMB8 |
| 640.126622 | −4.8286362 | 0.46343413 | −10.41925 | 2.03E−25 | 1.21E−22 | ENSG00000204261.4 | PSMB8-AS1 |
| 2511.33775 | −8.0371901 | 0.57142058 | −14.065279 | 6.21E−45 | 1.22E−41 | ENSG00000240065.3 | PSMB9 |
| 18731.2377 | −6.3263209 | 0.46506367 | −13.603129 | 3.84E−42 | 5.93E−39 | ENSG00000168394.9 | TAP1 |
| 570.015888 | −5.7272936 | 0.84758985 | −6.7571522 | 1.41E−11 | 3.16E−09 | ENSG00000242574.4 | HLA-DMB |
| 559.811367 | −3.2372583 | 0.52399538 | −6.1780284 | 6.49E−10 | 1.22E−07 | ENSG00000204257.10 | HLA-DMA |
| 29.0759101 | −7.7892911 | 1.01437527 | −7.6789047 | 1.60E−14 | 4.53E−12 | ENSG00000204252.8 | HLA-DOA |
| 810.438861 | −9.3025844 | 0.93688774 | −9.9292413 | 3.11E−23 | 1.55E−20 | ENSG00000231389.3 | HLA-DPA1 |
| 327.952734 | −7.6127896 | 0.96046756 | −7.9261288 | 2.26E−15 | 7.30E−13 | ENSG00000223865.6 | HLA-DPB1 |
| 4879.31406 | −2.0254996 | 0.19103012 | −10.603038 | 2.88E−26 | 1.84E−23 | ENSG00000231925.7 | TAPBP |
| 1175.11719 | −5.2054755 | 0.4203783 | −12.382836 | 3.24E−35 | 3.07E−32 | ENSG00000010030.9 | ETV7 |
| 33.1358206 | −5.2760081 | 0.70230564 | −7.5124103 | 5.80E−14 | 1.54E−11 | ENSG00000224666.2 | NA |
| 3.31472881 | −2.0066922 | 0.4837896 | −4.1478614 | 3.36E−05 | 0.00339993 | ENSG00000213500.3 | NA |
| 5088.27069 | −0.9246883 | 0.16455638 | −5.6192793 | 1.92E−08 | 3.01E−06 | ENSG00000024048.6 | UBR2 |
| 37.6087858 | −3.2630942 | 0.55388441 | −5.891291 | 3.83E−09 | 6.47E−07 | ENSG00000224944.1 | CASC6 |
| 56.8816262 | −4.6922947 | 0.85457407 | −5.4907992 | 4.00E−08 | 6.06E−06 | ENSG00000203797.5 | DDO |
| 732.609123 | −1.1819726 | 0.28231822 | −4.1866676 | 2.83E−05 | 0.00292848 | ENSG00000078269.9 | SYNJ2 |
| 1.55500118 | −4.0285437 | 1.02762111 | −3.9202617 | 8.85E−05 | 0.00822388 | ENSG00000231654.1 | RPS6KA2-AS1 |
| 532.62702 | −1.4001105 | 0.21155994 | −6.6180321 | 3.64E−11 | 7.86E−09 | ENSG00000026297.11 | RNASET2 |
| 161.894218 | −1.5996433 | 0.31471243 | −5.082873 | 3.72E−07 | 4.96E−05 | ENSG00000197146.2 | NA |
| 1918.85287 | −0.5210831 | 0.10107232 | −5.155547 | 2.53E−07 | 3.44E−05 | ENSG00000106346.7 | USP42 |
| 692.082136 | −1.3168481 | 0.21681848 | −6.073505 | 1.25E−09 | 2.28E−07 | ENSG00000106100.6 | NOD1 |
| 700.715265 | −9.2213323 | 0.748937 | −12.312561 | 7.75E−35 | 6.90E−32 | ENSG00000177409.7 | SAMD9L |
| 871.52677 | −2.4114168 | 0.48894446 | −4.9318829 | 8.14E−07 | 0.00010403 | ENSG00000169871.8 | TRIM56 |
| 1255.61783 | −2.7357726 | 0.45902627 | −5.9599478 | 2.52E−09 | 4.39E−07 | ENSG00000260336.1 | NA |
| 126.691963 | −7.3811574 | 0.70999783 | −10.396028 | 2.58E−25 | 1.52E−22 | ENSG00000146859.6 | TMEM140 |
| 25.5438465 | −7.7472933 | 0.90402379 | −8.5697893 | 1.04E−17 | 3.91E−15 | ENSG00000272941.1 | NA |
| 4554.11932 | −1.1999617 | 0.27101971 | −4.427581 | 9.53E−06 | 0.00107684 | ENSG00000105939.8 | ZC3HAV1 |
| 10.5761715 | −2.1618685 | 0.5040651 | −4.2888676 | 1.80E−05 | 0.00197612 | ENSG00000229677.1 | NA |
| 3195.50436 | −2.7583447 | 0.35460882 | −7.7785566 | 7.34E−15 | 2.20E−12 | ENSG00000059378.8 | PARP12 |
| 60.9845409 | −3.3484516 | 0.72112359 | −4.6433811 | 3.43E−06 | 0.00040605 | ENSG00000146955.6 | RAB19 |
| 33.8923367 | −3.0329438 | 0.70850819 | −4.2807463 | 1.86E−05 | 0.00204199 | ENSG00000170379.15 | TCAF2 |
| 9.20748198 | −3.1605291 | 0.67298411 | −4.6962908 | 2.65E−06 | 0.000319 | ENSG00000253882.2 | LOC154761 |
| 5821.92064 | −1.5208844 | 0.20488385 | −7.4231543 | 1.14E−13 | 3.00E−11 | ENSG00000013374.11 | NUB1 |
| 9.10350204 | −3.8378642 | 0.75565576 | −5.0788526 | 3.80E−07 | 5.05E−05 | ENSG00000245025.2 | NA |
| 5.89586011 | −3.2650575 | 0.80040108 | −4.0792767 | 4.52E−05 | 0.00445393 | ENSG00000189233.7 | NUGGC |
| 85.9924625 | −5.8245751 | 1.02434738 | −5.6861327 | 1.30E−08 | 2.09E−06 | ENSG00000131203.8 | IDO1 |
| 201.486824 | −3.934137 | 0.58025616 | −6.7800003 | 1.20E−11 | 2.72E−09 | ENSG00000177182.6 | CLVS1 |
| 18.4232328 | −3.7534423 | 0.95356038 | −3.9362398 | 8.28E−05 | 0.00776911 | ENSG00000104432.8 | IL7 |
| 40.0969891 | −6.2772804 | 0.91720405 | −6.8439302 | 7.70E−12 | 1.80E−09 | ENSG00000261618.1 | NA |
| 1671.47134 | −3.4301598 | 0.43245576 | −7.9318166 | 2.16E−15 | 7.05E−13 | ENSG00000178685.9 | PARP10 |
| 484.75493 | −6.1190722 | 0.5372126 | −11.390411 | 4.67E−30 | 3.43E−27 | ENSG00000120217.9 | CD274 |
| 2.59773443 | −3.5303666 | 0.84313639 | −4.1871833 | 2.82E−05 | 0.00292848 | ENSG00000171855.5 | IFNB1 |
| 2166.4722 | −2.9387063 | 0.34824514 | −8.4386142 | 3.21E−17 | 1.18E−14 | ENSG00000107201.5 | DDX58 |
| 2460.55558 | −0.8648321 | 0.22167751 | −3.9013072 | 9.57E−05 | 0.00875676 | ENSG00000196116.6 | TDRD7 |
| 55.5305318 | −6.4834699 | 0.70390569 | −9.2107082 | 3.24E−20 | 1.40E−17 | ENSG00000134470.15 | IL15RA |
| 3904.27922 | −1.4818012 | 0.27359097 | −5.4161187 | 6.09E−08 | 9.13E−06 | ENSG00000123240.12 | OPTN |
| 270.659916 | −2.3150457 | 0.44555306 | −5.1958924 | 2.04E−07 | 2.81E−05 | ENSG00000026103.15 | FAS |
| 1.25412727 | −3.7326989 | 0.95415389 | −3.9120512 | 9.15E−05 | 0.00845511 | ENSG00000238991.1 | NA |
| 1739.45949 | −6.1515865 | 0.514384 | −11.959133 | 5.82E−33 | 5.03E−30 | ENSG00000119922.7 | IFIT2 |
| 2895.70187 | −9.3980365 | 0.66708086 | −14.088302 | 4.48E−45 | 9.41E−42 | ENSG00000119917.9 | IFIT3 |
| 1783.16077 | −3.4648817 | 0.56757239 | −6.1047397 | 1.03E−09 | 1.89E−07 | ENSG00000185745.8 | IFIT1 |
| 7.06767141 | −3.8632825 | 0.71844249 | −5.3773023 | 7.56E−08 | 1.12E−05 | ENSG00000232709.1 | NA |
| 1767.5877 | −5.830895 | 0.5407361 | −10.783254 | 4.13E−27 | 2.76E−24 | ENSG00000197142.6 | ACSL5 |
| 1253.83182 | −1.8331978 | 0.31888467 | −5.7487799 | 8.99E−09 | 1.47E−06 | ENSG00000165806.15 | CASP7 |
| 195.217173 | −8.7626723 | 0.85570067 | −10.240348 | 1.31E−24 | 6.86E−22 | ENSG00000185885.11 | IFITM1 |
| 190.638355 | −7.436322 | 0.71744576 | −10.364995 | 3.58E−25 | 2.06E−22 | ENSG00000142089.11 | IFITM3 |
| 991.706394 | −5.5860285 | 0.67097977 | −8.3251817 | 8.42E−17 | 2.94E−14 | ENSG00000132109.8 | TRIM21 |
| 72.3003417 | −4.922578 | 0.85754005 | −5.7403477 | 9.45E−09 | 1.53E−06 | ENSG00000132256.14 | TRIM5 |
| 3077.15661 | −9.3940584 | 0.60583783 | −15.505896 | 3.16E−54 | 1.03E−50 | ENSG00000132274.11 | TRIM22 |
| 2709.03913 | −0.5299723 | 0.13450523 | −3.9401611 | 8.14E−05 | 0.0076677 | ENSG00000129084.13 | PSMA1 |
| 14249.8676 | −0.8150764 | 0.13232549 | −6.1596324 | 7.29E−10 | 1.36E−07 | ENSG00000049449.4 | RCN1 |
| 7.54173208 | −2.8404026 | 0.54180001 | −5.2425296 | 1.58E−07 | 2.23E−05 | ENSG00000186714.8 | CCDC73 |
| 5676.28523 | −4.9824212 | 0.39867998 | −12.497295 | 7.72E−36 | 8.10E−33 | ENSG00000156587.11 | UBE2L6 |
| 518.398909 | −10.056317 | 0.70023575 | −14.361331 | 9.05E−47 | 2.22E−43 | ENSG00000149131.11 | SERPING1 |
| 2457.44307 | −2.678702 | 0.67410147 | −3.9737371 | 7.08E−05 | 0.0067363 | ENSG00000166801.11 | FAM111A |
| 50.3618844 | −6.0480775 | 0.61517047 | −9.8315471 | 8.23E−23 | 3.97E−20 | ENSG00000110446.5 | SLC15A3 |
| 3.48776546 | −4.1556644 | 1.07305484 | −3.8727419 | 0.00010762 | 0.00969881 | ENSG00000133317.10 | LGALS12 |
| 4100.66705 | −9.0747418 | 1.01336658 | −8.9550435 | 3.40E−19 | 1.43E−16 | ENSG00000133321.6 | RARRES3 |
| 96.8776213 | −2.5306473 | 0.59454003 | −4.2564792 | 2.08E−05 | 0.00225143 | ENSG00000176485.6 | PLA2G16 |
| 3.66146029 | −3.1830397 | 0.78671369 | −4.0459951 | 5.21E−05 | 0.0050855 | ENSG00000168070.7 | MAJIN |
| 962.04231 | −7.3622989 | 0.46283001 | −15.907134 | 5.65E−57 | 2.37E−53 | ENSG00000168062.5 | BATF2 |
| 284.466048 | −5.6984073 | 0.77655333 | −7.3380759 | 2.17E−13 | 5.54E−11 | ENSG00000110092.3 | CCND1 |
| 5416.7121 | −1.4970147 | 0.31554667 | −4.7441942 | 2.09E−06 | 0.00025626 | ENSG00000137496.13 | IL18BP |
| 86.5292868 | −4.0242499 | 0.85082427 | −4.7298249 | 2.25E−06 | 0.00027281 | ENSG00000196954.8 | CASP4 |
| 86.3483135 | −7.9066477 | 0.86481221 | −9.1426179 | 6.10E−20 | 2.60E−17 | ENSG00000137752.18 | CASP1 |
| 25.9930397 | −6.2886232 | 1.05434286 | −5.9644955 | 2.45E−09 | 4.32E−07 | ENSG00000204397.3 | CARD16 |
| 849.23957 | −2.5475479 | 0.33379712 | −7.6320246 | 2.31E−14 | 6.41E−12 | ENSG00000139192.7 | TAPBPL |
| 9.48792178 | −5.5895318 | 0.89294738 | −6.2596431 | 3.86E−10 | 7.61E−08 | ENSG00000121380.8 | BCL2L14 |
| 3.24141877 | −4.8500677 | 1.06904876 | −4.5368068 | 5.71E−06 | 0.0006529 | ENSG00000179256.2 | SMCO3 |
| 18.2976937 | −1.7479676 | 0.44787647 | −3.9027895 | 9.51E−05 | 0.00873049 | ENSG00000135436.4 | FAM186B |
| 12431.5081 | −2.0275945 | 0.2347174 | −8.6384498 | 5.70E−18 | 2.20E−15 | ENSG00000170581.9 | STAT2 |
| 21.1578879 | −2.4451024 | 0.40574918 | −6.0261427 | 1.68E−09 | 3.01E−07 | ENSG00000252206.1 | NA |
| 311.077937 | −1.4626037 | 0.29367108 | −4.9804146 | 6.34E−07 | 8.18E−05 | ENSG00000127311.5 | HELB |
| 30.0156117 | −1.2865089 | 0.32016928 | −4.0182148 | 5.86E−05 | 0.00563028 | ENSG00000238528.1 | NA |
| 247.042569 | −2.6458946 | 0.49241437 | −5.3733091 | 7.73E−08 | 1.13E−05 | ENSG00000136048.9 | DRAM1 |
| 131.700791 | −8.2184601 | 0.57560089 | −14.278053 | 3.00E−46 | 6.78E−43 | ENSG00000256262.1 | USP30-AS1 |
| 5476.88062 | −0.588256 | 0.11981832 | −4.9095661 | 9.13E−07 | 0.0001151 | ENSG00000135148.7 | TRAFD1 |
| 3169.8844 | −4.6208221 | 0.61422448 | −7.5230185 | 5.35E−14 | 1.43E−11 | ENSG00000089127.8 | OAS1 |
| 12648.6455 | −2.2774593 | 0.35800182 | −6.3615857 | 2.00E−10 | 4.02E−08 | ENSG00000111331.8 | OAS3 |
| 216.868607 | −5.4424779 | 0.69471996 | −7.83406 | 4.72E−15 | 1.46E−12 | ENSG00000111335.8 | OAS2 |
| 18.3800514 | −6.7369471 | 1.25685336 | −5.3601696 | 8.31E−08 | 1.20E−05 | ENSG00000271579.1 | NA |
| 43.2106926 | −6.1285625 | 0.93678133 | −6.5421484 | 6.06E−11 | 1.28E−08 | ENSG00000135114.8 | OASL |
| 8307.14509 | −1.0867987 | 0.25996776 | −4.1805134 | 2.91E−05 | 0.00299832 | ENSG00000102699.5 | PARP4 |
| 45.5348666 | −3.8365002 | 0.83169206 | −4.6128854 | 3.97E−06 | 0.00046669 | ENSG00000133106.10 | EPSTI1 |
| 191.472605 | −1.9666907 | 0.25971048 | −7.5726274 | 3.66E−14 | 9.95E−12 | ENSG00000225131.1 | NA |
| 3431.95089 | −1.102044 | 0.27933395 | −3.9452564 | 7.97E−05 | 0.0075306 | ENSG00000088387.13 | DOCK9 |
| 49.0661707 | −5.4359894 | 0.68797885 | −7.9013903 | 2.76E−15 | 8.73E−13 | ENSG00000258573.1 | LOC254028 |
| 3280.29066 | −0.4424716 | 0.0999832 | −4.4254591 | 9.62E−06 | 0.00108332 | ENSG00000129472.8 | RAB2B |
| 11499.7042 | −1.9641717 | 0.19175798 | −10.242973 | 1.27E−24 | 6.80E−22 | ENSG00000092010.10 | PSME1 |
| 91.9770188 | −2.0307372 | 0.2984238 | −6.804877 | 1.01E−11 | 2.32E−09 | ENSG00000259321.1 | NA |
| 12925.7557 | −1.9188476 | 0.18353687 | −10.454835 | 1.39E−25 | 8.52E−23 | ENSG00000100911.9 | PSME2 |
| 1679.37002 | −0.6386818 | 0.10727146 | −5.9538838 | 2.62E−09 | 4.50E−07 | ENSG00000092098.12 | RNF31 |
| 350.31012 | −0.9955612 | 0.13589203 | −7.3261189 | 2.37E−13 | 6.00E−11 | ENSG00000259529.1 | NA |
| 1075.01997 | −2.5372825 | 0.18699831 | −13.568478 | 6.16E−42 | 9.05E−39 | ENSG00000213928.4 | IRF9 |
| 6117.2793 | −0.4148686 | 0.10562551 | −3.9277307 | 8.58E−05 | 0.00802347 | ENSG00000100567.8 | PSMA3 |
| 18.0897127 | −3.3925602 | 0.83429812 | −4.0663644 | 4.78E−05 | 0.00467654 | ENSG00000258733.1 | NA |
| 3258.13525 | −1.4165974 | 0.2635785 | −5.37448 | 7.68E−08 | 1.13E−05 | ENSG00000133943.16 | DGLUCY |
| 7.78292119 | −2.3367098 | 0.56283204 | −4.1517 | 3.30E−05 | 0.00336661 | ENSG00000100599.11 | RIN3 |
| 1248.98521 | −3.3997434 | 0.44030388 | −7.7213568 | 1.15E−14 | 3.32E−12 | ENSG00000165949.8 | IFI27 |
| 26865.7274 | −3.3203114 | 0.31664319 | −10.485971 | 1.00E−25 | 6.26E−23 | ENSG00000140105.13 | WARS |
| 59.2840167 | −1.6392512 | 0.42091433 | −3.8945008 | 9.84E−05 | 0.00895054 | ENSG00000092529.18 | CAPN3 |
| 132.331846 | −4.4428556 | 0.5082297 | −8.7418259 | 2.29E−18 | 8.99E−16 | ENSG00000229474.2 | PATL2 |
| 68403.5012 | −3.9272295 | 0.3187407 | −12.321079 | 6.98E−35 | 6.40E−32 | ENSG00000166710.13 | B2M |
| 4581.40129 | −2.8291886 | 0.59294627 | −4.771408 | 1.83E−06 | 0.00022583 | ENSG00000185880.8 | TRIM69 |
| 12123.3488 | −0.9131382 | 0.23333414 | −3.913436 | 9.10E−05 | 0.00843327 | ENSG00000129003.11 | VPS13C |
| 4122.34976 | −1.4211082 | 0.19288726 | −7.3675586 | 1.74E−13 | 4.48E−11 | ENSG00000140464.15 | PML |
| 202.940872 | −2.4161568 | 0.51573592 | −4.6848721 | 2.80E−06 | 0.00033593 | ENSG00000172183.10 | ISG20 |
| 23.3577616 | −1.7431538 | 0.44453151 | −3.9213277 | 8.81E−05 | 0.00821357 | ENSG00000153060.3 | TEKT5 |
| 4778.32652 | −10.077203 | 0.57823472 | −17.427531 | 5.10E−68 | 2.50E−64 | ENSG00000179583.13 | CIITA |
| 605.072586 | −1.2855528 | 0.21963732 | −5.8530709 | 4.83E−09 | 8.10E−07 | ENSG00000263013.1 | NA |
| 26.1881728 | −3.296014 | 0.50390652 | −6.5409235 | 6.11E−11 | 1.28E−08 | ENSG00000262222.1 | NA |
| 131.509115 | −3.4741967 | 0.41263991 | −8.4194393 | 3.78E−17 | 1.37E−14 | ENSG00000263179.1 | NA |
| 54.2659379 | −2.5853025 | 0.45540541 | −5.6769253 | 1.37E−08 | 2.19E−06 | ENSG00000185338.4 | SOCS1 |
| 3471.69997 | 0.5496168 | 0.13884871 | 3.9583862 | 7.55E−05 | 0.00715146 | ENSG00000166851.10 | PLK1 |
| 60.4547195 | −1.9735391 | 0.46409399 | −4.2524556 | 2.11E−05 | 0.00228385 | ENSG00000238045.5 | NA |
| 34.7824075 | −3.3387462 | 0.72552655 | −4.601825 | 4.19E−06 | 0.00048827 | ENSG00000261690.1 | NA |
| 2420.95015 | −1.512441 | 0.26949564 | −5.6121167 | 2.00E−08 | 3.12E−06 | ENSG00000013364.14 | MVP |
| 237.376364 | −0.6585723 | 0.1568655 | −4.1983244 | 2.69E−05 | 0.00280149 | ENSG00000260083.1 | MIR762HG |
| 14.8405197 | −3.5848016 | 0.7118158 | −5.0361367 | 4.75E−07 | 6.23E−05 | ENSG00000261644.1 | NA |
| 4.68503844 | −3.4126103 | 0.79507258 | −4.2921998 | 1.77E−05 | 0.00195401 | ENSG00000260929.1 | NA |
| 2257.34807 | −1.5322666 | 0.37465617 | −4.089794 | 4.32E−05 | 0.00428547 | ENSG00000125148.6 | MT2A |
| 16039.9939 | −5.849547 | 0.40669045 | −14.383291 | 6.59E−47 | 1.76E−43 | ENSG00000140853.11 | NLRC5 |
| 82.810706 | −2.0392713 | 0.50541767 | −4.0348239 | 5.46E−05 | 0.00531596 | ENSG00000006210.6 | CX3CL1 |
| 261.583856 | −2.4136265 | 0.36874533 | −6.5455107 | 5.93E−11 | 1.26E−08 | ENSG00000261884.2 | NA |
| 682.250859 | −5.2554908 | 0.37426172 | −14.042288 | 8.59E−45 | 1.58E−41 | ENSG00000205220.7 | PSMB10 |
| 190.143607 | −2.9565557 | 0.50979992 | −5.7994433 | 6.65E−09 | 1.11E−06 | ENSG00000168404.8 | MLKL |
| 85.7583476 | −3.7045545 | 0.48111356 | −7.6999586 | 1.36E−14 | 3.88E−12 | ENSG00000135697.5 | BCO1 |
| 991.673424 | −2.9271174 | 0.69264352 | −4.2260084 | 2.38E−05 | 0.00250491 | ENSG00000140968.6 | IRF8 |
| 1681.2406 | −11.472196 | 0.89793118 | −12.776253 | 2.23E−37 | 2.62E−34 | ENSG00000132530.12 | XAFI |
| 13.212154 | −6.4922487 | 1.04408182 | −6.2181417 | 5.03E−10 | 9.66E−08 | ENSG00000177294.6 | FBXO39 |
| 531.467061 | −2.738824 | 0.40607712 | −6.7445907 | 1.53E−11 | 3.42E−09 | ENSG00000168961.12 | LGALS9 |
| 4.52450765 | −4.9910286 | 1.25610408 | −3.9734196 | 7.08E−05 | 0.0067363 | ENSG00000108700.4 | CCL8 |
| 4.53977031 | −4.0396178 | 0.80113166 | −5.0423895 | 4.60E−07 | 6.08E−05 | ENSG00000204952.2 | FBXO47 |
| 4.1330195 | −4.9504425 | 0.79253744 | −6.24632 | 4.20E−10 | 8.18E−08 | ENSG00000196859.3 | KRT39 |
| 255.593602 | −3.0628481 | 0.4341717 | −7.0544628 | 1.73E−12 | 4.21E−10 | ENSG00000108771.8 | DHX58 |
| 14861.2825 | −0.9181196 | 0.14395173 | −6.3779683 | 1.79E−10 | 3.64E−08 | ENSG00000168610.10 | STAT3 |
| 684.66562 | −6.4538253 | 0.41286384 | −15.631849 | 4.42E−55 | 1.62E−51 | ENSG00000068079.3 | IFI35 |
| 7028.73307 | −1.0481409 | 0.2187598 | −4.7912868 | 1.66E−06 | 0.00020543 | ENSG00000121060.10 | TRIM25 |
| 29.8807978 | −1.4454119 | 0.37077991 | −3.8983015 | 9.69E−05 | 0.00883861 | ENSG00000263120.1 | NA |
| 522.496308 | −0.6047704 | 0.13586846 | −4.4511464 | 8.54E−06 | 0.00096889 | ENSG00000267248.1 | LOC100996660 |
| 404.150661 | −1.2051601 | 0.2948651 | −4.0871577 | 4.37E−05 | 0.00431985 | ENSG00000070540.8 | WIPI1 |
| 9.69419508 | −2.4189706 | 0.57416894 | −4.2129945 | 2.52E−05 | 0.00263487 | ENSG00000204277.1 | LINC01993 |
| 217.61773 | −1.7967774 | 0.41989098 | −4.2791522 | 1.88E−05 | 0.00204903 | ENSG00000184557.3 | SOCS3 |
| 11338.1837 | −1.5018416 | 0.21036246 | −7.1393042 | 9.38E−13 | 2.32E−10 | ENSG00000108679.8 | LGALS3BP |
| 20298.4722 | −3.097208 | 0.32796313 | −9.443769 | 3.60E−21 | 1.65E−18 | ENSG00000173821.15 | RNF213 |
| 14.0120087 | −3.3344613 | 0.62169488 | −5.3635013 | 8.16E−08 | 1.18E−05 | ENSG00000262979.1 | NA |
| 103.733534 | 1.31984285 | 0.33788745 | 3.90616121 | 9.38E−05 | 0.00863661 | ENSG00000263069.1 | LOC100294362 |
| 29.5532967 | −7.0061412 | 1.62697915 | −4.3062268 | 1.66E−05 | 0.00184808 | ENSG00000261520.1 | DLGAP1-AS5 |
| 3634.68146 | −0.9927847 | 0.24188875 | −4.1043029 | 4.06E−05 | 0.0040526 | ENSG00000141682.11 | PMAIP1 |
| 47.2825426 | −5.7424034 | 0.77560719 | −7.4037522 | 1.32E−13 | 3.44E−11 | ENSG00000131142.9 | CCL25 |
| 1027.55832 | −3.6015707 | 0.60466183 | −5.9563387 | 2.58E−09 | 4.46E−07 | ENSG00000130813.13 | C19orf66 |
| 769.525802 | −6.6326508 | 0.49009453 | −13.533411 | 9.93E−42 | 1.27E−38 | ENSG00000090339.4 | ICAM1 |
| 99.7971261 | −2.5813817 | 0.56732392 | −4.550102 | 5.36E−06 | 0.00062022 | ENSG00000214212.4 | C19orf38 |
| 16.2891687 | −3.9111664 | 0.77584797 | −5.0411505 | 4.63E−07 | 6.10E−05 | ENSG00000102575.6 | ACP5 |
| 5.65368256 | −5.0802512 | 0.76398561 | −6.6496687 | 2.94E−11 | 6.39E−09 | ENSG00000269720.1 | CCDC194 |
| 1465.43962 | −9.5673043 | 0.50400551 | −18.982539 | 2.38E−80 | 2.33E−76 | ENSG00000130303.8 | BST2 |
| 24.7986922 | −6.5672834 | 0.71073077 | −9.2401845 | 2.46E−20 | 1.10E−17 | ENSG00000269640.1 | NA |
| 20.4257187 | −4.5611438 | 0.65221217 | −6.9933436 | 2.68E−12 | 6.46E−10 | ENSG00000096996.11 | IL12RB1 |
| 20.9119476 | −5.881068 | 0.66708521 | −8.8160671 | 1.19E−18 | 4.71E−16 | ENSG00000226025.5 | LGALS17A |
| 257.465194 | −6.0098552 | 0.65202829 | −9.2171694 | 3.05E−20 | 1.34E−17 | ENSG00000079385.17 | CEACAM1 |
| 3617.34738 | −0.6282935 | 0.15084294 | −4.1652164 | 3.11E−05 | 0.00318427 | ENSG00000104805.11 | NUCB1 |
| 67.3670225 | −2.0119212 | 0.30788119 | −6.5347326 | 6.37E−11 | 1.33E−08 | ENSG00000090554.8 | FLT3LG |
| 13.5194097 | −1.7008527 | 0.33439742 | −5.0863214 | 3.65E−07 | 4.90E−05 | ENSG00000273189.1 | NA |
| 418.010719 | −2.7725499 | 0.65719821 | −4.2187424 | 2.46E−05 | 0.00257777 | ENSG00000172296.8 | SPTLC3 |
| 4121.33373 | −2.6625379 | 0.38237701 | −6.9631221 | 3.33E−12 | 7.89E−10 | ENSG00000101347.7 | SAMHD1 |
| 5756.43438 | −1.1094117 | 0.21271951 | −5.2153737 | 1.83E−07 | 2.54E−05 | ENSG00000124201.10 | ZNFX1 |
| 16.4918121 | −5.3465586 | 0.94936237 | −5.6317364 | 1.78E−08 | 2.82E−06 | ENSG00000124256.10 | ZBP1 |
| 2137.70314 | −1.000098 | 0.1635005 | −6.1167889 | 9.55E−10 | 1.78E−07 | ENSG00000060491.12 | OGFR |
| 2335.57646 | −1.7389911 | 0.41134107 | −4.2276136 | 2.36E−05 | 0.00249606 | ENSG00000130589.12 | HELZ2 |
| 107.035452 | −5.048124 | 0.90933839 | −5.5514251 | 2.83E−08 | 4.34E−06 | ENSG00000183486.8 | MX2 |
| 2566.0096 | −7.1681582 | 0.85475042 | −8.3862588 | 5.02E−17 | 1.80E−14 | ENSG00000157601.9 | MXI |
| 1062.40299 | −1.899427 | 0.31079309 | −6.1115483 | 9.87E−10 | 1.82E−07 | ENSG00000184979.9 | USP18 |
| 6387.49478 | −0.4100632 | 0.09658705 | −4.2455294 | 2.18E−05 | 0.00233946 | ENSG00000100225.13 | FBXO7 |
| 14516.1354 | −9.0410367 | 0.6667928 | −13.55899 | 7.01E−42 | 9.45E−39 | ENSG00000221963.5 | APOL6 |
| 1197.87044 | −8.0935685 | 0.79005105 | −10.244361 | 1.25E−24 | 6.80E−22 | ENSG00000128284.15 | APOL3 |
| 3093.12736 | −5.6746799 | 0.91164287 | −6.2246742 | 4.83E−10 | 9.33E−08 | ENSG00000100336.13 | APOL4 |
| 8380.66124 | −4.6866688 | 0.40591266 | −11.546003 | 7.73E−31 | 5.98E−28 | ENSG00000128335.9 | APOL2 |
| 2807.98718 | −10.559232 | 0.5449176 | −19.377667 | 1.19E−83 | 1.75E−79 | ENSG00000100342.16 | APOL1 |
| 22.4106156 | −3.1395815 | 0.67445118 | −4.6550166 | 3.24E−06 | 0.00038534 | ENSG00000179750.11 | APOBEC3B |
| 66.2645125 | −4.4633645 | 0.74215208 | −6.0140834 | 1.81E−09 | 3.20E−07 | ENSG00000243811.3 | APOBEC3D |
| 293.226914 | −4.3018455 | 0.66165903 | −6.5016048 | 7.95E−11 | 1.63E−08 | ENSG00000128394.12 | APOBEC3F |
| 341.237288 | −5.0794124 | 0.85572467 | −5.9358022 | 2.92E−09 | 4.99E−07 | ENSG00000239713.3 | APOBEC3G |
| 54.88906 | −2.3335954 | 0.54716557 | −4.2648798 | 2.00E−05 | 0.0021764 | ENSG00000183569.13 | SERHL2 |
| 27.2866699 | −1.6451284 | 0.38777135 | −4.2425217 | 2.21E−05 | 0.00235276 | ENSG00000231010.1 | NA |
| 506.414767 | −0.7657969 | 0.1465539 | −5.22536 | 1.74E−07 | 2.43E−05 | ENSG00000185753.8 | CXorf38 |
| 29.178915 | −2.158004 | 0.52150097 | −4.1380632 | 3.50E−05 | 0.0035362 | ENSG00000260802.1 | SERTM2 |
| 380.599844 | −0.7462903 | 0.14919842 | −5.0019991 | 5.67E−07 | 7.41E−05 | ENSG00000156500.10 | FAM122C |
| 1790.83039 | −6.264907 | 0.40442087 | −15.491058 | 3.99E−54 | 1.17E−50 | ENSG00000155962.8 | CLIC2 |
| 5.10923122 | −4.4251083 | 0.81863278 | −5.4054863 | 6.46E−08 | 9.64E−06 | ENSG00000225008.1 | NA |

To investigate the degree of heterogeneity in the HLA I downregulation observed in bulk transcriptome sequencing of MCC cells, HLA expression was evaluated for two fresh MCC biopsies (MCC350 [MCPyV−] and MCC336 [MCPyV+]) by high-throughput droplet-based single-cell transcriptome analysis. Reads from both samples were aligned to hg19 using Cellranger, and transcript quantities were analyzed using the Seurat pipeline (see Example 1). Following sample QC, the cells were grouped using Louvain clustering. From a total of xx 15,808 cells (mean 4231.9 genes/cells) identified across the two samples, 7 distinct transcriptionally defined clusters were detected. Immune cells, identified by CD45 expression, comprised cluster 6, while clusters 0-5 were MCC cells, identified by the expression of SOX2, SYP, and ATOH1 (FIGS. 2F, 2G). All MCC clusters displayed nearly absent HLA-B, TAP1/2, PSMB8/9, and NLRC5 expression and low HLA-A and -C expression (FIGS. 2F, 2H), consistent with the aforementioned bulk characterization of surface HLA I expression in MCC cell lines. By contrast, cluster 6 (immune cells) displayed a mean 8.22-fold higher expression of HLA-A, -B, and -C transcripts.

Given the marked RNA- and protein-level downregulation of multiple class I genes, it was first sought to identify a possible genetic basis for these observations. By WES, none of the MCC lines harbored any notable somatic mutations in 27 canonical HLA I pathway genes with the exception of an HLA-F and -H mutation in MCC-320 (Table 9). While a total of 32 mutations were detected in interferon genes those included within the REACTOME_INTERFERON_SIGNALING gene set), only 2 were predicted as probably damaging by Polyphen and no mutations were detected in the canonical interferon genes IFNGR1/2, JAK1/2, STAT1, and IRF1/2 (Table 9). However, copy number loss of NLRC5 was detected in 5 of 8 lines for which copy number variation analysis was performed (FIG. 21 and data not shown). NLRC5 is a transcriptional activator of several HLA I pathway components (i.e., HLA-A, -B, -C, -E, -F, B2M, TAP1, and PSMB9 (LMP2)) that localizes to conserved S/X/Y regions in the promoters of these genes, and positive correlation were observed between NLRC5 and these other class I genes in the MCC lines (FIG. 2J). By analysis of matched whole-genome bisulfite sequencing, NLRC5 promoter hypermethylation compared to other class I antigen presentation genes was also detected (FIG. 2K, Table 10), suggesting an additional mechanism by which NLRC5 might be suppressed in MCC. Consistent with these observations, NLRC5 copy number loss and promoter methylation have been recently recognized as a common alteration across diverse cancers. To further explore the epigenetic landscape, chromatin accessibility at class I pathway genes was also investigated using ATAC-Seq data (FIGS. 2L-2N). While analysis of NLRC5 was found inconclusive, a lack of clear peaks at the transcription start sites of HLA-A and HLA-B compared to controls such as keratinocytes, B cells, and NLRC5 the melanoma line 501-Mel (FIGS. 2L, 2O)-C and NLRC5 (FIG. 2L) was observed, providing further evidence for epigenetic silencing of class 1 genes.

TABLE 9

| | Mutated Gene | Mutation Type | Sample with Mutation | Viral Status |
|---|---|---|---|---|
| Class 1 | HLA-F | Missense | MCC-320 T, CL | negative |
| Pathway | | | | |
| Interferon | ADAR | Missense | MCC-320 T, CL | negative |
| Pathway | NUP210 | Multi-Hit | MCC-320 T, CL | negative |
| | SEC13 | Missense | MCC-320 T, CL | negative |
| | TRIM3 | Missense | MCC-320 T, CL | negative |
| | TRIM29 | Frame Shift Deletion | MCC-320 T | negative |
| | DDX58 | Missense | MCC-350 T, CL | negative |
| | HLA-DQA1 | Nonsense | MCC-350 T, CL | negative |
| | TRIM6 | Missense | MCC-350 T, CL | negative |
| | GBP6 | Missense | MCC-350 T | negative |
| | CAMK2G | Missense | MCC-301 T | positive |
| | PDE12 | Missense | MCC-301 T | positive |
| | PIAS1 | Missense | MCC-367 | positive |
| | IFNA16 | Frame Shift Deletion | MCC-2314 CL | positive |
| | NUP155 | Splice Site | MCC-2314 CL | positive |
| | NUP214 | Missense | MCC-2314 T | positive |
| | TRIM8 | Frame Shift Insertion | MCC-2314 T | positive |
| | EIF4E | Missense | MCC-336 | Positive |
| Tumor | RB1 | Nonsense | MCC-350 T, CL | Negative |
| Suppressors | RB1 | Splice Site | MCC-320 T, CL | Negative |
| | TP53 | Missense | MCC-350 T, CL | Negative |
| | TP53 | Missense | MCC-320 T, CL | Negative |
| | NOTCH1 | Missense | MCC-320 T, CL | Negative |

TABLE 10

| | | | | | | | |
|---|---|---|---|---|---|---|---|
| MCC-282 | MCC-290 | MCC-301 | MCC-320 | MCC-336 | MCC-350 | MCC-367 | MCC-2334 |

| Sample | Gene | neg | neg | pos | neg | pos | neg | pos | pos |
|---|---|---|---|---|---|---|---|---|---|
| Viral Status IFN | | IFN- | IFN- | IFN- | IFN- | non-IFN- | non-IFN- | IFN- | IFN- |
| Responsiveness | | responsive | responsive | responsive | responsive | responsive | responsive | responsive | responsive |
| | HLA-A | 0.153 | 0.362 | 0.149 | 0.579 | 0.276 | 0.141 | 0.236 | 0.129 |
| | HLA-B | 0.19 | 0.172 | 0.149 | 0.202 | 0.149 | 0.135 | 0.116 | 0.24 |
| | HLA-C | 0.192 | 0.199 | 0.188 | 0.234 | 0.113 | 0.158 | 0.127 | 0.191 |
| | B2M | 0.221 | 0.325 | 0.188 | 0.4 | 0.257 | 0.301 | 0.151 | 0.21 |
| | TAP1 | 0.467 | 0.44 | 0.497 | 0.491 | 0.491 | 0.416 | 0.404 | 0.525 |
| | TAP2 | 0.577 | 0.667 | 0.689 | 0.678 | 0.55 | 0.553 | 0.597 | 0.655 |
| | TAPBP | 0.202 | 0.517 | 0.257 | 0.534 | 0.56 | 0.429 | 0.135 | 0.39 |
| | PSMB8 | 0.315 | 0.359 | 0.346 | 0.351 | 0.394 | 0.339 | 0.259 | 0.411 |
| | PSMB9 | 0.167 | 0.202 | 0.175 | 0.191 | 0.224 | 0.172 | 0.126 | 0.23 |
| | NLRCS | 0.785 | 0.776 | 0.839 | 0.824 | 0.785 | 0.651 | 0.727 | 0.842 |

### Example 4: IFN-γ-Induced HLA I Upregulation is Associated with Shifts in the HLA Peptidome

Diminished expression of HLA I would be expected to result in a lower number and diversity of HLA-presented peptides in MCC, impacting the immunogenicity of the tumor. Indeed, using standard workflows for direct detection of class I bound peptides by mass spectrometry, following immunoprecipitation of tumor cell lysates with a pan-H-LA class I antibody (FIG. 3A; see Methods), similarly low total peptide counts at baseline were detected in parental tumors and cell lines. Following IFN-γ stimulation, a median 25-fold increase in the abundance of class I bound peptides was detected across 4 cell lines (FIG. 3B). Whereas a high level of correlation in the immunopeptidome amino acid signature was observed between the tumors and cell lines at baseline, lower correlations were observed between cell lines before and after IFN-y treatment (FIG. 3C). While cell line peptidomes shared more than 50% of their peptides with the corresponding tumor peptidomes (FIG. 3D) and showed similar binding motifs, IFN-γ treatment appeared to alter the overall motif (FIG. 3E). To further explore these observations, the most likely HLA-allele to which the identified peptides were bound was inferred. The inferred frequencies of peptides presented on each class I HLA allele were similar between corresponding tumors and cell lines (FIGS. 3F, 3G). When comparing cell lines exposed or not to IFN-γ, dramatic changes were observed in the frequencies of peptides mapping to each HLA allele, most notably an increase in HLA-B-presented peptides (FIGS. 3E, 3H). This is consistent with previous observations that interferons upregulate HLA-B more strongly than HLA-A attributable to HLA-B having two interferon-responsive elements in its promoter.

For the MCPyV+ lines, it was hypothesized that this upregulation of HLA I following IFN-y stimulation would lead to increased ability to present MCPyV-specific epitopes. Indeed, for the MCPyV+ line, MCC-367, a peptide sequence was detected derived from the OBD domain of LT (TSDKAIELY), which was predicted as a strong binder to HLA*A0101 of that cell line (rank=0.018, HLAthena) (Sarkizova et al. (2020) Nature 38 (2): 199-209).

### Example 5: Complementary Genome-Scale Loss- and Gain-of-Function Screens Identify Known and Novel Potential Regulators of HLA I Expression in MCC

Although NLRC5 copy number loss and promoter methylation was identified as a contributory factor in enforcing the silencing of the HLA I pathway, at least three lines (MCC-290, -301, -320) exhibited normal NLRC5 copy number and had low levels of HLA I expression. Hence, identification of alternative pathways and mechanisms underlying the high degree of HLA I surface loss and downregulation of multiple class I components was attempted.

To this end, paired genome-scale CRISPR-KO loss-of-function and open reading frame (ORF) gain-of-function screens were designed to systematically identify novel regulators of HLA I surface expression in MCC. These screens were conducted in the virus-positive MCC-301 line due to its robust growth rate, and also because of its low mutational background, enabling focus to be placed on the role of deregulated genes. It was also hypothesized that the novel impacted pathways identified in this MCPyV+ context would be mirrored in MCPyV− MCC, wherein HLA I suppression might be achieved through somatic mutations affecting these same pathways. MCC-301 cells were transduced at a low multiplicity of infection (MOI) with genome-wide lentiviral libraries containing either ORF or Cas9+sgRNA constructs. After staining cells with an anti-HLA-ABC antibody, the HLA I-high and HLA I-low populations underwent fluorescence activated cell sorting (FACS)-based cell sorting isolation, with each screen performed in biologic triplicate (FIG. 4A). Of note, transduction with the ORF library but not the CRISPR library led to a population-wide increase in HLA I surface expression, presumably due to interferon secretion from interferon-related gene ORF-expressing cells. This was an ORF library-specific effect and not due to the process of lentiviral transduction, as GFP-transduced cells did not exhibit an increase in surface HLA I (FIG. 4B). The median construct log 2-fold change from 3 replicates for the ORF screen, while for the CRISPR screen, one replicate that had poor sample quality was discarded and the remaining two high-quality replicates were averaged (FIG. 4C).

The ORF screen produced 75 hits with a greater than twofold increase in median log 2-fold change (enrichment in HLA I-high vs HLA I-low). As expected, these hits were highly enriched for interferon and HLA I pathway genes by Gene Set Enrichment Analysis (GSEA) (Subramanian et al. (2005) PNAS USA 102 (43): 15545-50) (FIG. 4D, Tables 1 and 2). In some embodiments, the twofold change (e.g., increase or decrease) is used to select biomarkers from within Tables 1, 2, 3, 4, or 5. The top hit was IFNG, with interferon signaling pathway genes comprising four of the top 12. In addition, HLA-B and -C were hits #10 and #38, respectively. Strikingly, MYCL was found to be the top negative hit (FIG. 4D). MYCL is a central transcription factor in MCPyV+ MCC, as ST binds and recruits MYCL to the EP400 chromatin modifier complex to enact widespread epigenetic changes necessary for oncogenesis.

The CRISPR-KO screen also identified several known components of the HLA class I pathway. Sequencing of the CRISPR library-transduced cells prior to FACS confirmed that adequate sgRNA representation was present (FIG. 4C). Positive and negative hits were then ranked according to the STARS algorithm (Doench et al. (2016) supra. The top negative hit (gene whose knockout resulted in the highest enrichment in the HLA I-low population) was TAPBP (FIG. 4E, Tables 1 and 2), a key class I pathway component that acts as a chaperone for partially folded HLA I heavy chains and facilitates binding between unbound HLA I and TAP. Other notable negative hits were also identified including IFN pathway gene IRF1 (#21) and class I genes CALR (#84) and B2M (#141), while GSEA showed enrichment for gene sets related to protein translation. (FIG. 4E; Tables 1 and 2).

Within the CRISPR positive hits, several components of the Polycomb repressive complex PRC1.1 were recurrently identified, including the top two hits of the screen: BCORL1 (#1), USP7 (#2), and PCGF1 (#46). For each, >4.5-fold enrichment was observed for at least 2 sgRNAs of these genes (FIG. 4G). PRC1.1 is a noncanonical Polycomb repressive complex that silences gene expression through ubiquitination of H2AK119 in CpG islands. In addition to the screen hits, other components of the PRC1.1 complex include KDM2B, SKP1, RING1A/B, RYBP/YAF2, and BCOR (interchangeable with BCORL1).

The notable positive and negative hits in both screens exhibited high concordance between at least 2 replicates (FIG. 4I). To validate ORF screen positive hits, single ORF overexpression lines were in MCC-301 were generated, focusing on the top 71 hits not related to interferon or HLA I pathways. Using flow cytometry, 8 of 71 candidate hits (11.3%) were confirmed to upregulate MFI (HLA-ABC) by greater than 2-fold compared to GFP-transduced control while also maintaining viability after transduction, including TFEB, CXorf67, and YY1 (FIG. 411).

For the CRISPR screen, a targeted validation was performed of top hits by generating a series of MCC-301 KO lines using the two highest-scoring sgRNAs against PRC1.1 components BCORL1, PCGF1, and USP7. Genome editing by Cas9 was confirmed by Sanger sequencing using TIDE (Brinkman et al. (2014) Nucleic Acids Research 42 (22): e168) FIG. 4I, and functional knockdown was confirmed by Western blot or qRT-PCR. Knockout of each gene increased surface HLA I expression by flow cytometry relative to MCC-301 transduced with a control non-targeting sgRNA (FIG. 4J). In aggregate, review of the top hits across the parallel screens revealed several hits related to Polycomb repressive complexes [PRC1.1 components USP7, PCGF1, and BCORL1; ORF hits CXorf67 and YY1; PRC2 components EED and SUZ12 (CRISPR positive hit #162 and #409)] and to the ST-MYCL-EP400 complex [MYCL and CRISPR positive hits BRD8 (#51), DMAP1 (#93), KAT5 (#619), and EP400 (#886)]. Since both MYCL and PRC1.1 component USP7 encode proteins that have been reported to directly interact with MCPyV (Cheng et al. (2017) PLoS Pathogens 13 (10): e1006668) (Czech-Sioli et al. (2020) J. Virology 94 (5) doi.org/10.1128/JVI.01638-19) (FIG. 4K), these were therefore selected for more in-depth characterization. Additionally, a TIDE analysis of PRC1.1 KO lines was performed, which allowed determination of the percentage of cells with indels in each knockout line (FIG. 4L). Analysis of the MCC-301 CRISPR HLA-ABC screen and the independent K562 screen (Burr et al. 2019), identified overlapping hits.

### Example 6: MYCL Mediates HLA I Suppression in MCC

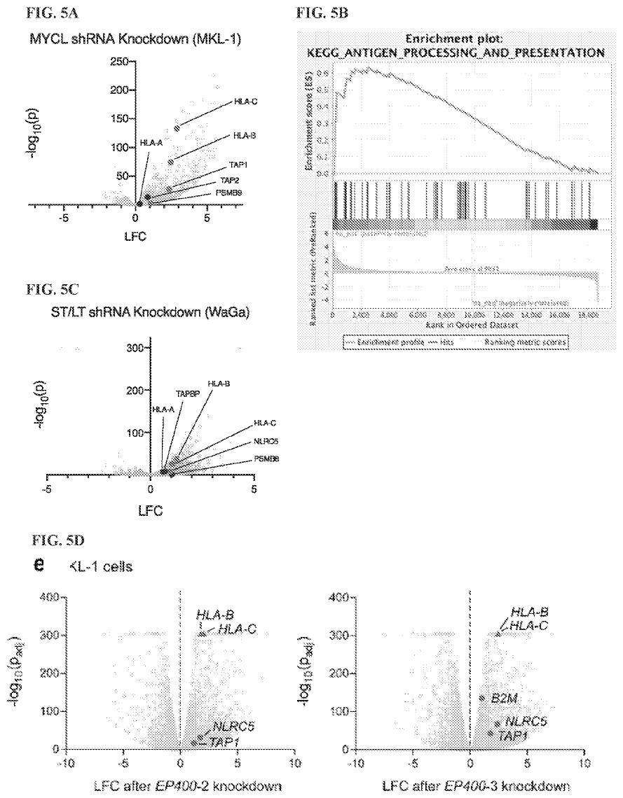

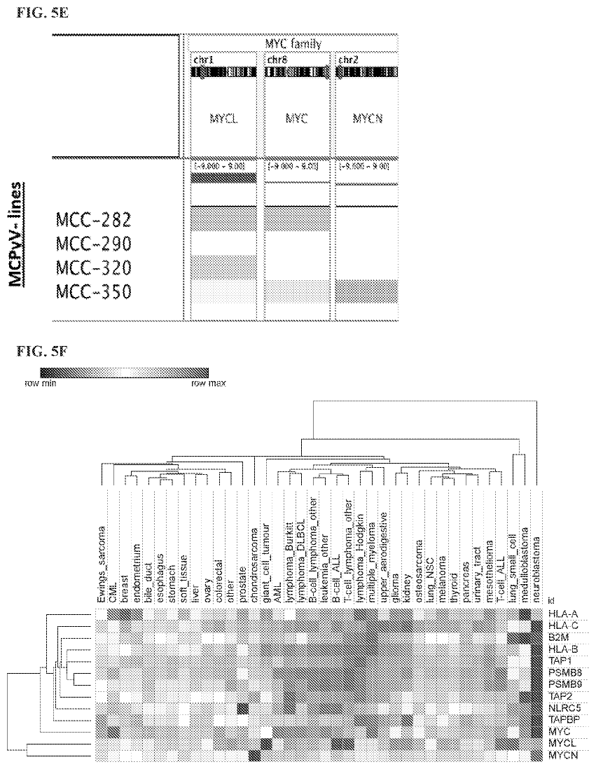

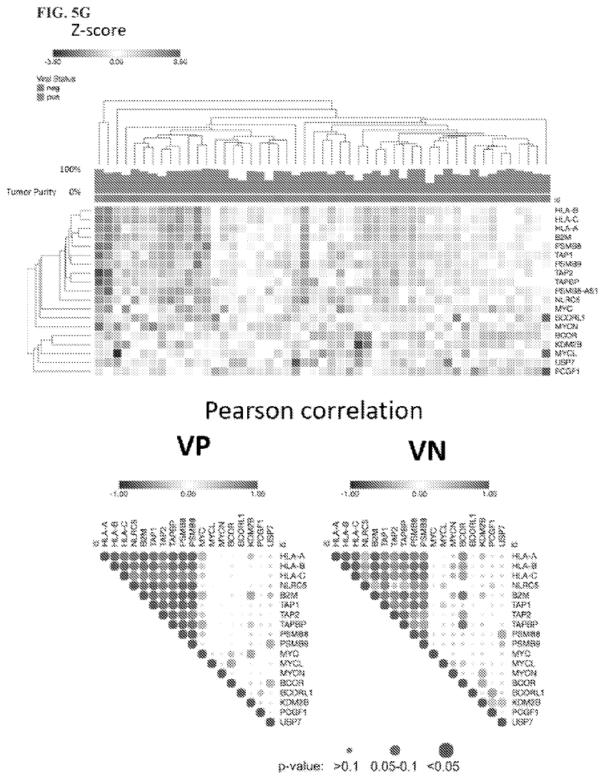

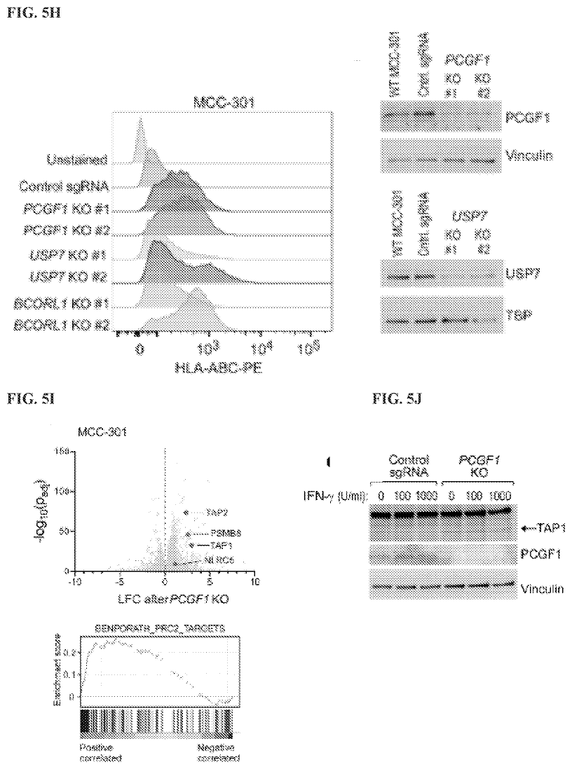

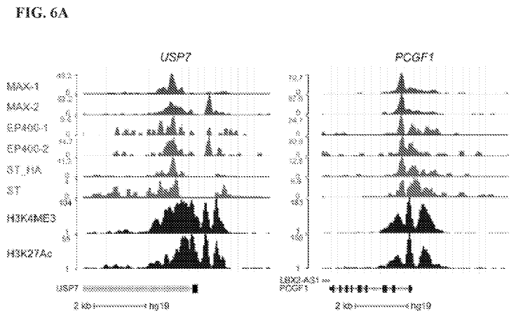

Since MYCL overexpression reduced HLA I in the HLA I-high IMR90 fibroblast line, it was investigated if MYCL inactivation was sufficient to restore class I in an HLA I-low MCC line. A MYCL shRNA was introduced into the MCPyV+MKL-1 cell line and MYCL knockdown was compared to a scrambled shRNA control. RNA-seq analysis of these knockdown lines revealed a >2-fold increase in expression of several class I genes including HLA-B, —C, and TAP1 with enrichment for the signature of antigen processing/presentation by GSEA (q=0.04; FIGS. 5A, 5B and data not shown). Since ST binds and potentiates MYCL function through the ST-EP400-MYCL complex (Cheng et al. (2017) PLoS Pathogens 13 (10): e1006668), it was suspected that viral antigen inactivation might also upregulate class I. in fact, after transducing the WaGa cell line (MCPyV+) with an shRNA that targets shared exons of ST and LT leading to inactivation of both MCPyV viral antigens, a similar but more modest upregulation of class I genes was observed, including >1.5 fold increase in HLA-B, -C, and NLRC5 (FIG. 5C and data not shown). Moreover, knockdown of EP400 in MKL-1 with two different shRNAs resulted in >4-fold increases in HLA-B and HLA-C (FIG. 5D). These findings directly implicate expression of ST-EP400-MYCL complex components with HLA I regulation in MCC.

### Example 7: MYCL is Relevant to MCPyV− MCC and Other Cancers

To determine if the HLA I-suppressive effects of MYCL generalized to viral-negative MCC as well, the copy number status of MYCL was evaluated in MCPyV− MCC. Copy number gain of chromosome 1p, encompassing MYCL, was previously reported as one of the more common copy number alterations in MCC. Indeed, 3 of the 4 virus-negative MCC lines gain in MYCL copy number (log 2 copy number ratio 0.22-0.64) (FIG. 5E), suggesting a mechanism by which MCPyV− MCC may enhance MYCL signaling in the absence of viral antigens. To determine if this mechanism might be employed by other cancers, publicly available RNA-seq data was queried from the Cancer Cell Line Encyclopedia (Ghandi et al. (2019) Nature 569 (7757): 503-8). Cancer cell lines with lower expression of HLA I pathway components such as SCLC and neuroblastoma also frequently featured overexpression of MYC family members MYCL and MYCN, respectively (FIG. 5F).

### Example 8: PRC1.1 Components are Regulated by MYCL

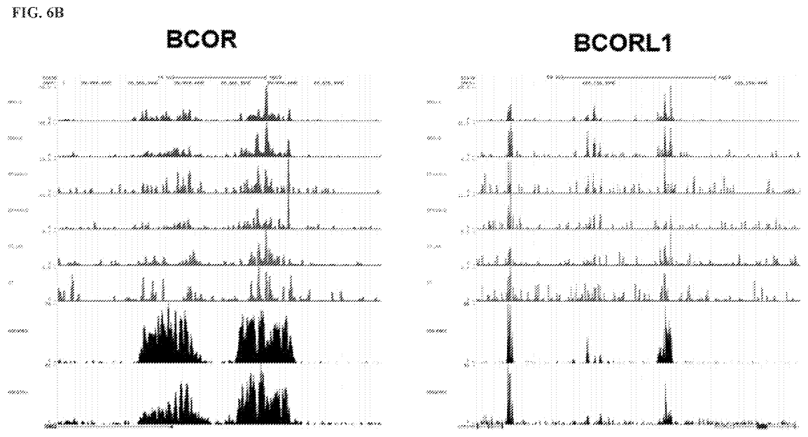

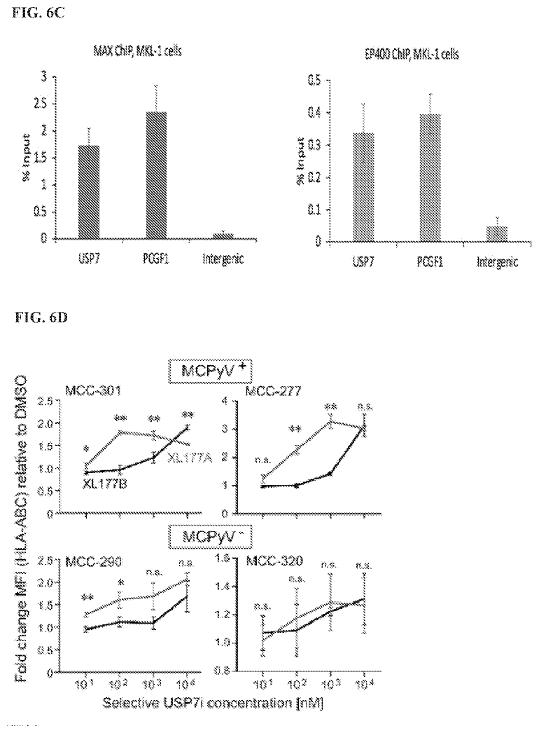

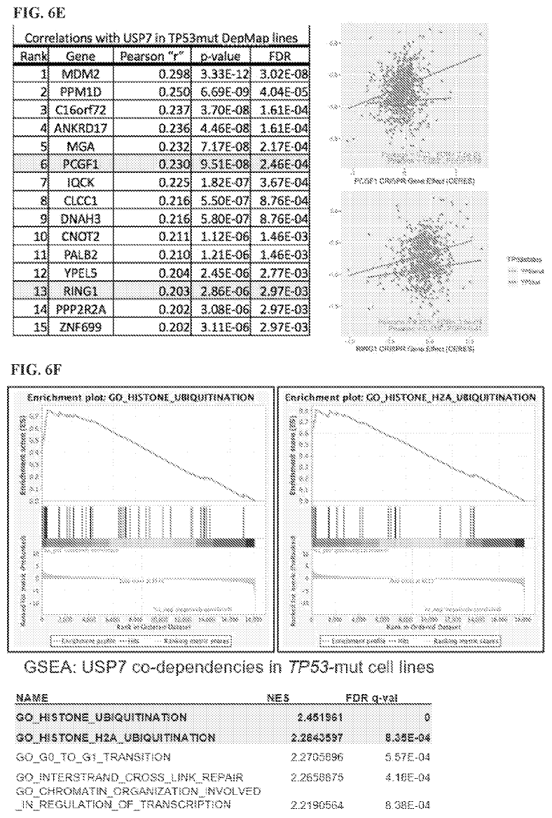

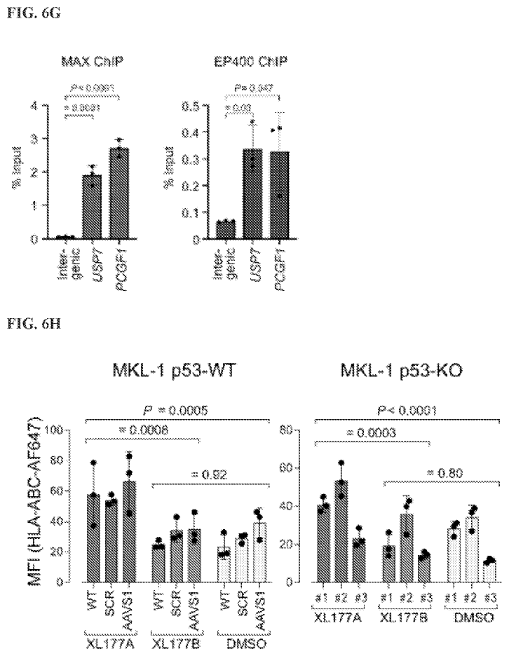

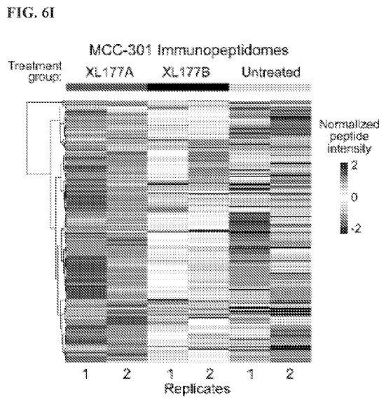

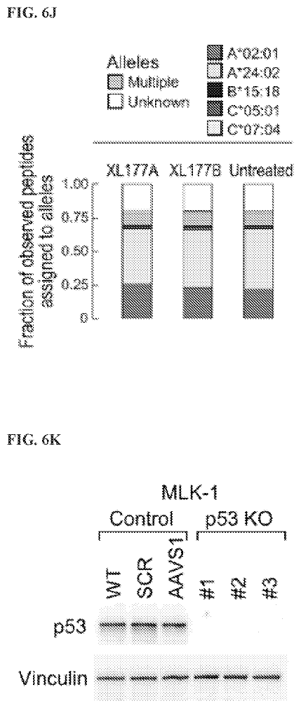

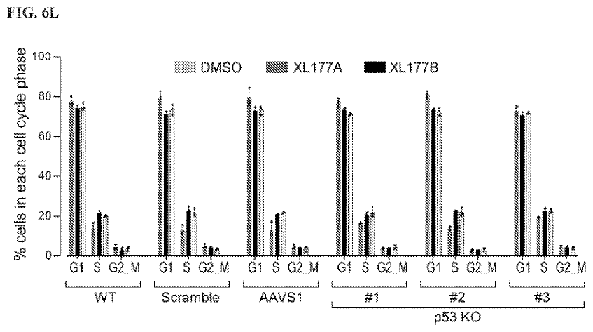

To examine the association between expression of HLA class I genes and the screen hits in an RNA-seq cohort of 52 MCC tumors, including both MCPyV+ and MCPyV− were examined. To account for the potential of immune cell infiltration confounding the bulk class I expression data, ESTIMATE (Yoshihara et al. (2013) Nature Communications 4: 2612) was used to calculate tumor purity. While MYCL was not associated with class I expression in this cohort, a negative correlation was observed between several class I genes and PRC1.1 components KDM2B and USP7 in MCPyV+ MCC, and BCOR and USP7 in MCPyV− MCC (p<0.05; FIG. 5G). These findings motivated further investigation of the relationship of PRC1.1 to MYCL and to HLA class I genes. Upon reanalysis of previously generated ChIP-seq data (Cheng et al. (2017) supra, it was observed that components of the ST-MYCL-EP400 complex bind to the promoter of PRC1.1 genes USP7 and PCGF1, but not BCOR or BCORL1 (FIGS. 6A, 6B). The binding of MAX and EP400 to USP7 and PCGF1 was subsequently confirmed by ChIP qPCR in MKL-1 cells (FIG. 6C). These results suggested the possibility that PRC1.1 could act downstream of MYCL in regulating HLA I.

### Example 9: Pharmacologic Inhibition of USP7 Restores HLA I in MCPyV+ and MCPyV− MCC in a PRC1.1-Dependent Manner

To investigate the role of PRC1.1 on HLA Class I regulation in MCC, focused was placed on USP7, for which selective small molecule inhibitors have been developed. The activity of XL177A, a potent and irreversible USP7 inhibitor, was compared to that with XL177B, the enantiomer compound that exhibits 500-fold less potency, serving as a control (Schauer et al. (2020) Scientific Reports 10 (1): 5324). Two MCPyV+ lines (MCC-301, MCC-277) and two MCPyV− lines (MCC-290, MCC-320) were treated for 3 days at varying inhibitor concentrations. At 100 nM, a mean 1.89-fold (range 1.60-2.27) increase was observed in expression of surface HLA class I by flow cytometry relative to DMSO in three lines (MCC-277, -290, -301) in response to XL177A, which was significantly different from the control compound XL177B (p-values 0.002-0.02; the fold change refers to XL177A relative to DMSO, and the p value is a comparison of XL177A vs XL177B) (FIG. 6D). MCC-320 did not exhibit a statistically significant increase in HLA I expression.

Since USP7 is known to have myriad functions (for example, regulation of p53 through MDM2 deubiquitination) and since its role in PRC1.1 was only recently discovered (Maat et al. (2019) bioRxiv. doi.org/10.1101/221093), it was investigated whether the effect of USP7 on HLA I was in fact mediated by PRC1.1. Data within the Cancer Dependency Map (Dempster et al. (2019) bioRxiv. doi.org/10.1101/720243); Meyers et al. (2017) Nature Genetics 49 (12): 1779-84) was leveraged to identify genes whose survival dependency correlated with that of USP7 across cancer cell lines, with the rationale that survival co-dependency implies that such genes may function within the same complex or pathway. While TP53-WT lines did not exhibit codependency between USP7 and Polycomb genes, TP53-mutant lines showed a high correlation between USP7 and PRC1.1 genes PCGF1 and RING1, (correlation rank of 6 and 13 and p<0.0003 and 0.003, respectively) (FIG. 6E and data not shown). Furthermore, GSEA analysis revealed the process of histone ubiquitination as the most enriched gene set within USP7 co-dependent genes in TP53-mutant cell lines (FIG. 6F and data not shown). These results indicate that although its primary role is p53 regulation, USP7 also plays an important role in PRC1.1.

If USP7 were acting through PRC1.1, inhibition of USP7 would not impact HLA I expression were it applied to a line lacking expression of a competent PRC1.1 complex. USP7 was inhibited in the MCC-301 PCGF1-KO line, and results were confirmed by flow cytometry for surface class I (FIG. 6E). Based on the ChIP-seq evidence that the EP400-ST-MYCL complex binds to the USP7 promoter, MYCL was suspected to also be at least partially dependent on USP7 for HLA suppression.

Understanding regulators of HLA I in MCC has the potential to provide broad insights mechanisms of class I antigen presentation suppression in the setting of both viral infection and cancer. Through generation and genomic characterization of 11 robust MCC cell lines, it was shown that loss of surface HLA I is underpinned by transcriptional downregulation of multiple class I pathway genes and alterations to NLRC5. Through genome-wide screens in an MCPyV+ MCC line, novel upstream regulators of HLA I, including PRC1.1 and MYCL, were identified, which are believed to mediate viral antigen-driven HLA I suppression.

Low surface HLA I and transcriptional loss of TAP1/2 and PSMB8/9 (LMP7/2) in MCC have been demonstrated. As presented herein, downregulation of these class I genes was confirmed, and the HLA class I transcriptional activator NLRC5 was shown to also be a target for alteration, exhibiting both copy number (CN) loss and promoter methylation in many of the new MCC cell lines. NLRC5 expression is known to correlate with expression of several class I genes across many cancers, and NLRC5 CN loss was observed in 28.6% of a TCGA cohort of 7,730 cancer patients. However, given that NLRC5 is still expressed in these MCC lines, albeit at lower levels relative to normal tissue controls, it was hypothesized that there could be other epigenetic regulators orchestrating class I downregulation in MCC, perhaps due to viral antigen signaling. Pharmacologic inhibition of such an HLA regulator could increase the immunogenicity of MCC tumors, as evidenced by the ability to detect HLA-presented viral epitopes following IFN-γ treatment demonstrated herein.

Thus, genome-scale gain- and loss-of-function screens were performed that found that PRC1.1 and MYCL are negative regulators of HLA I surface expression in MCC. MYCL is an intriguing candidate regulator of HLA I that is activated in virus-positive MCC by ST antigen and frequently amplified in virus-negative MCC. Additionally, MYC and MYCN are known to suppress HLA I surface expression in melanoma and neuroblastoma, respectively. Based upon the known interaction between MYCL and ST and the experiments presented herein demonstrating that knockdown of either one upregulates class I genes, MCPyV could suppress class I through ST interactions with MYCL. Given the ability of ST to recruit MYCL and the EP400 complex to transactivate a large number of downstream target genes, it was hypothesized that one or more of these target genes contributes to repression of MHC I. Two notable ST-MYCL-EP400 downstream target genes are USP7 and PCGF1 both of which are CRISPR screen hits and components component of the PRC1.1 complex.

PRC1.1 belongs to a family of Polycomb complexes, which are repressive chromatin modifiers that act in tandem. In the traditional model, PRC2 deposits repressive H3K27me3 marks on unmethylated CpG islands, and these marks subsequently recruit canonical PRC1, which ubiquitinates H2AK119. Several non-canonical PRC1 variant complexes have also been identified, one of which is PRC1.1, which can target unmethylated CpG islands independently of PRC2. Polycomb complexes are important in cancer, having been implicated as both oncogenes and tumor suppressors, and PRC2 inhibitors have shown promise in early clinical trials in lymphomas and sarcomas. The connection between Polycomb complexes and HLA class I regulation is a new and promising development: PRC2 was recently identified as a repressor of HLA I through an independent CRISPR screen in the leukemia cell line K562 (Burr et al. (2019) Cancer Cell 36 (4): 385-401.e8), and this work establishes a novel connection to the PRC1.1 complex as well. Within the context of MCC, it has been shown that epigenetic modifiers such as histone deacetylase inhibitors can upregulate class I, but this work identifies some of the specific players involved in crafting the epigenetic landscape around class I genes. Burr et al validated PRC2 KO-mediated HLA I upregulation in one MCC line as well, lending further credence to the significance of Polycomb complexes in MCC. The screen presented herein and Burr et al.'s screens identified several overlapping hits, including PCGF1, perhaps suggesting a coordination between PRC1.1 and PRC2 to suppress class I. The studies presented herein show class I upregulation with a small-molecule USP7 inhibitor and provide an avenue for pharmacologic targeting of PRC1.1.

However, it is important to consider that the role of PRC1.1 and MYCL in HLA I regulation may be context- and cell-type-dependent. Although PRC1.1 targets unmethylated CpG islands, it is unknown if there are additional factors that refine its specificity. While the genome-wide screens were performed in a single, MCPyV⁺ MCC line (MCC-301), an inverse correlation was observed between HLA class I and several PRC1.1 components within a large cohort of 52 MCC tumors. Moreover, the identification of another Polycomb complex in Burr et al 2019's K562 CRISPR screen further indicates a convergent biology.

HLA I loss is an important mechanism of immune evasion in viral infections and cancer, and a better understanding of these mechanisms can help identify targets for restoration of HLA I. Through genome-scale screens in MCC, PRC1.1 and MYCL among many others were identified as novel suppressors of HLA I surface expression. These results identify therapeutic targets and highlight two ways by which MCPyV viral antigens may modulate HLA class I genes.

### Example 10: Materials and Methods for Examples 11-19

a. Data and Code Availability

DbGaP submissions (accession number phs002260) for WES, RNA-seq, scRNA-seq, and WGBS are currently pending and will be made publicly available. All analysis code for whole exome sequencing analysis, RNA-seq analysis, MCPyV viral transcript detection, and WGBS promoter signal extraction are made available in a Github repository under an MIT license at github.com/kdkorthauer/MCC. The original mass spectra for all proteomics and immunopeptidomics experiments, tables of peptide spectrum matches for immunopeptidome experiments, and the protein sequence databases used for searches have been deposited in the public proteomics repository MassIVE (massive.ucsd.edu) and are accessible at ftp://MSV000087251@massive.ucsd.edu with username: MSV000087251 password: modulation.

b. Experimental Model and Subject Details

### Human Subjects:

For the MCC tumor samples, patients were consented to under IRB protocol #09-156 at the Dana-Farber Cancer Institute. Patients' clinical annotations are listed in Table 6.

### Cell Lines:

Newly derived MCC cell lines were cultured at 37° C. in NeuroCult NS-A Human Proliferation Medium (StemCell Technologies) supplemented with 0.02% Heparin (StemCell Technologies), 20 ng/ml hEGF (Miltenyi Biotec) and 20 ng/ml hFGF-2 (Miltenyi Biotec). Cell line sexes are described in Table 6. Cell lines were authenticated as MCC through immunohistochemical staining using antibodies against CK20 and SOX2 (FIG. 1B; FIG. 1P). Cell lines were authenticated as derivatives of original tumor samples by HLA typing, which was available for 7 of the 11 lines (See below). MKL-1 and WaGa lines were grown in RPMI-1640 medium supplemented with 10% fetal bovine serum (FBS; Gibco) and 100 penicillin/streptomycin (Gibco).

HLA typing.
HLA typing for 7 of the 11 MCC lines for which whole-exome sequencing data.

| | HLA | | | | |
|---|---|---|---|---|---|
| Patient | Allele | Tumor | | Cell Line | |
| MCC- | HLA- | HLA-A*11:01:01 | HLA-A*32:01:01 | HLA-A*ll:01:01 | HLA-A*32:01:01 |
| 277 | A | | | | |
| | HLA- | HLA-B*14:01:01 | HLA-B*51:01:01 | HLA-B*14:01:01 | HLA-B*51:01:01 |
| | B | | | | |
| | HLA- | HLA-C*15:02:01 | HLA-C*08:02:01 | HLA-C*15:02:01 | HLA-C*08:02:01 |
| | C | | | | |
| MCC- | HLA- | HLA-A*24:02:01:01 | HLA-A*02:01:01:01 | HLA-A*24:02:01:01 | HLA-A*02:01:01:01 |
| 301 | A | | | | |
| | HLA- | HLA-B*15:18:01 | HLA-B*44:02:01:01 | HLA-B*15:18:01 | HLA-B*44:02:01:01 |
| | B | | | | |
| | HLA- | HLA-C*07:04:01 | HLA-C*05:01:01:02 | HLA-C*07:04:01 | HLA-C*05:01:01:02 |
| | C | | | | |
| MCC- | HLA- | HLA-A*01:01:01:01 | HLA-A*25:01:01 | HLA-A*01:01:01:01 | HLA-A*25:01:01 |
| 320 | A | | | | |
| | HLA- | HLA-B*14:01:01 | HLA-B*18:01:01:02 | HLA-B*14:01:01 | HLA-B*18:01:01:02 |
| | B | | | | |
| | HLA- | HLA-C*12:03:01:01 | HLA-C*08:02:01 | HLA-C*12:03:01:01 | HLA-C*08:02:01 |
| | C | | | | |
| MCC- | HLA- | HLA-A*02:01:01:01 | HLA-A*02:01:01:01 | HLA-A*02:01:01:01 | HLA-A*02:01:01:01 |
| 336 | A | | | | |
| | HLA- | HLA-B*35:02:01 | HLA-B*52:01:01:02 | HLA-B*35:02:01 | HLA-B*52:01:01:02 |
| | B | | | | |
| | HLA- | HLA-C*12:02:02 | HLA-C*04:01:01:01 | HLA-C*12:02:02 | HLA-C*04:01:01:01 |
| | C | | | | |
| MCC- | HLA- | HLA-A*24:02:01:01 | HLA-A*29:02:01:01 | HLA-A*24:02:01:01 | HLA-A*29:02:01:01 |
| 350 | A | | | | |
| | HLA- | HLA-B*07:02:01 | HLA-B*08:01:01 | HLA-B*07:02:01 | HLA-B*08:01:01 |
| | B | | | | |
| | HLA- | HLA-C*07:02:01:01 | HLA-C*07:01:01:01 | HLA-C*07:02:01:01 | HLA-C*07:01:01:01 |
| | C | | | | |
| MCC- | HLA- | HLA-A*01:01:01:01 | HLA-A*31:01:02 | HLA-A*01:01:01:01 | HLA-A*31:01:02 |
| 367 | A | | | | |
| | HLA- | HLA-B*49:01:01 | HLA-B*51:01:01 | HLA-B*49:01:01 | HLA-B*51:01:01 |
| | B | | | | |
| | HLA- | HLA-C*12:03:01:01 | HLA-C*01:02:01 | HLA-C*12:03:01:01 | HLA-C*01:02:01 |
| | C | | | | |
| MCC- | HLA- | HLA-A*24:02:01:01 | HLA-A*02:01:01:01 | HLA-A*24:02:01:01 | HLA-A*02:01:01:01 |
| 2314 | A | | | | |
| | HLA- | HLA-B*07:02:01 | HLA-B*44:02:01:01 | HLA-B*07:02:01 | HLA-B*44:02:01:01 |
| | B | | | | |
| | HLA- | HLA-C*07:02:01:03 | HLA-C*05:01:01:02 | HLA-C*07:02:01:03 | HLA-C*05:01:01:02 |
| | C | | | | |

c. Generation of Tumor Cell Lines

Tumor samples were obtained from either patient biopsy or patient-derived xenografts. The tissue was minced manually, suspended in a solution of 2 mg/ml collagenase I (Sigma Aldrich), 2 mg/ml hyaluronidase (Sigma Aldrich) and 25 ug/ml DNase I (Roche Life Sciences), transferred to a 15 mL conical tube, and incubated on an orbital shaker at low speed for 30 min. After digestion, the single-cell suspension was passed through a 100 micron strainer, washed, and cultured in tissue culture flasks containing media from NeuroCult NS-A Human Proliferation Kit (StemCell Technologies) supplemented with 0.02% Heparin (StemCell Technologies), 20 ng/ml hEGF (Miltenyi Biotec) and 20 ng/ml hFGF-2 (Miltenyi Biotec). When available, excess tumor single cell suspensions were frozen in 90% FBS and 10% DMSO and banked in liquid nitrogen. Established cell lines were tested as mycoplasma free (Venor™ GeM Mycoplasma Detection Kit, Sigma Aldrich) and verified as MCC through immunohistochemical staining using antibodies against CK20 and SOX2. All MCC cell lines were maintained in media from NeuroCult NS-A Proliferation Kit supplemented with 0.02% heparin, 20 ng/mL hEGF, and 20 ng/mL hFGF2. Other media used for cell culture optimization included StemFlex (Gibco); Neurobasal (Gibco) supplemented with 0.02% heparin (StemCell Technologies), 20 ng/mL hEGF (Miltenyi Biotec), and 20 ng/mL hFGF2 (Miltenyi Biotec); DMEM GlutaMAX (Gibco) supplemented with 10% FBS (Gibco), 1% penicillin/streptomycin (Gibco), 1 mM sodium pyruvate (Life Technologies), 10 mM HEPES (Life Technologies), and 55 nM β-mercaptoethanol (Gibco); and RPMI-1640 (Gibco) supplemented with 20% FBS (Gibco) and 1% penicillin/streptomycin (Gibco).

d. Histology and Immunohistochemistry

All IHC was performed on the Leica Bond III automated staining platform. From the cell lines, up to 10 million MCC cells were pelleted, fixed in formaldehyde, washed with PBS, and mounted on a paraffin block. For single stains, 5-micron sections were cut and stained for SOX2 or CK20. The Leica Biosystems Refine Detection Kit was used with citrate antigen retrieval for SOX2 (Abcam #97959, polyclonal, 1:100 dilution) and with EDTA antigen retrieval for Cytokeratin 20 (CK20; Dako #M7019, clone Ks20.8, 1:50 dilution). For dual immunohistochemical staining of the archival tumor specimens, MCC marker SOX2 (CST, D6D9, 1:50 dilution; red chromogen) was used and either HLA class I (Abcam, EMR8-5, 1:6,000 dilution; brown chromogen) or HLA class II (Dako M0775, CR3/43, 1:750 dilution; brown chromogen) using an automated staining system (Bond III, Leica Biosystems) according to the manufacturer's protocol. The proportion of SOX2+ MCC cells that exhibited HLA I or HLA II membranous staining was evaluated by consensus of two board-certified pathologists.

e. Immunofluorescence

Staining was performed overnight on BOND RX fully automated stainers (Leica Biosystems). 5-μm thick formalin-fixed paraffin-embedded tumor tissue sections were baked for 3 hours at 60° C. before loading into the BOND RX. Slides were deparaffinized (BOND DeWax Solution, Leica Biosystems, Cat. AR9590) and rehydrated through a series of graded ethanol to deionized water. Antigen retrieval was performed in BOND Epitope Retrieval Solution 1 (ER1; pH 6) or 2 (ER2; pH 9) (Leica Biosystems, Cat. AR9961, AR9640) at 95° C. Deparaffinization, rehydration and antigen retrieval were all pre-programmed and executed by the BOND RX. Next, slides were serially stained with primary antibodies for: SOX2 (clone B6D9, Cell Signaling, dilution 1:200; Opal 690 1:100), CD8 (clone 4B11, Leica, dilution 1:200; Opal 480 1:150), PD-L1 (clone E1L3N, Cell Signaling, dilution 1:300; Opal 520 1:150), and PD-1 (clone EPR4877[2], Abcam, dilution 1:300; Opal 620 1:300) with ER1 for 20 min; and FOXP3 (clone D608R, Cell Signaling, dilution 1:100; Opal 570 1:300) with ER2 solution for 40 min. Each primary antibody was incubated for 30 minutes. Subsequently, anti-mouse plus anti-rabbit Opal Polymer Horseradish Peroxidase (Akoya Biosciences, Cat. ARH1001EA) was applied as a secondary label with an incubation time of 10 minutes. Signal for antibody complexes was labeled and visualized by their corresponding Opal Fluorophore Reagents (Akoya) by incubating the slides for 10 minutes. Slides were incubated in Spectral DAPI solution (Akoya) for 10 minutes, air dried, and mounted with Prolong Diamond Anti-fade mounting medium (Life Technologies, Cat. P36965) and imaged using the Vectra Polaris multispectral imaging platform (Vectra Polaris, Akoya Biosciences). Representative tumor regions of interest were identified by the pathologist and 2-6 fields of view were acquired per sample. Images were spectrally unmixed and cell identification was performed using the supervised machine learning algorithms within Inform 2.4 (Akoya) with pathologist supervision.

f. Flow Cytometry

Cells were dissociated with Versene and incubated with 5 μL Human TruStain FcX (Fc Receptor Blocking Solution; Biolegend #422302) per million cells in 100 μL at room temperature for 10 min. Fluorophore-conjugated antibodies or respective isotype controls were added and incubated for another 30 min at 4° C. Cells were then washed once with PBS and resuspended in PBS or 4% paraformaldehyde and analyzed on an LSR Fortessa cytometer. For HLA-I and HLA-II detection, the following antibodies were used: HLA-ABC (W6/32 clone) conjugated to PE (BioLegend #311406), APC (BioLegend #311410), or AF647 (Santa Cruz Biotechnology #sc32235 AF647), and HLA-DR-FITC (BioLegend #307604).

g. Whole Exome Sequencing and Mutation Calling

Genomic DNA samples were sheared using a Broad Institute-developed protocol optimized for ˜180 bp size distribution Kapa Hyperprep kits were used to construct libraries in a process optimized for somatic samples, including end repair, adapter ligation with forked adaptors containing unique molecular indexes, and addition of P5 and P7 sample barcodes via PCR. SPRI purification was performed and resulting libraries were quantified with Pico Green. Libraries were normalized and equimolar pooling was performed to prepare multiplexed sets for hybridization. Automated capture was performed, followed by PCR of the enriched DNA. SPRI purification was used for cleanup. Multiplex pools were then quantified with Pico Green and DNA fragment size was estimated using Bioanalyzer. Final libraries were quantitated by qPCR and loaded onto an Illumina flowcell across an adequate number of lanes to achieve ≥85% of target bases covered at ≥50× depth, with a range from 130-160× mean coverage of the targeted region.

Exome-sequencing BAM files were downloaded from the Broad Genomics Firecloud/Terra platform using the Google Cloud Storage command line tool gsutil version 4.5 (github.com/GoogleCloudPlatform/gsutil/). GATK version 4.1.2.0 was used to: (1) call mutations from reference on normal BAMs with Mutect2 command using a max MNP distance of 0, (2) build a panel of normals from VCF files of called normal mutations using the CreateSomaticPanelOfNormals command, and (3) call mutations between pairs of both tumor and cell line with compared to their respective normal counterpart using the Mutect2 command. For these steps, the following annotations were used: b37 reference sequence downloaded from ftp://ftp.broadinstitute.org/bundle/b37/human_g1k_v37.fasta, germline resource VCF downloaded from ftp://ftp.broadinstitute.org/bundle/beta/Mutect2/af-only-gnomad.raw.sites.b37.vcf.gz, and intervals list downloaded from https://github.com/broadinstitute/gatk/blob/master/src/test/resources/large/whole_exome_illu mina_coding_v1.Homo_sapiens_assembly9.targets.interval_list. Called variants were filtered with the GATK FilterMutectCalls command, and variants labeled as PASS were extracted and included in downstream analyses.

Next, VCF files of passing variants were annotated as MAF files using vcf2maf version 1.16.17 (downloaded from github.com/mskcc/vcf2maf/tree/5453f802d2f1f261708fe21c9d47b66d13e19737) and Variant Effect Predictor version 95 installed from github.com/Ensembl/ensembl-vep/archive/release/95.3.tar.gz. R Bioconductor package maftools⁷¹ was used to generate oncoplots of mutations by gene and sample. Patient HLA allotype was assessed using standard class I and class II PCR-based typing (Brigham and Women's Hospital Tissue Typing Laboratory).

h. Whole Genome Sequencing and Copy Number Analysis

Whole genome sequencing was performed by Admera Health. Reads were quality and adapter trimmed using TrimGalore with default settings. Trimmed reads were aligned against a fusion reference containing hg38 and MCPyV (NCBI accession number: NC_010277) using bowtie2-very-sensitive. Copy number variant analysis was performed with GATK4 CNV recommended practices. A panel of normals was generated from 17 normal blood whole genomes to call CNVs from tumors. All CNV calls that mapped to hg38 were visualized using the Integrative Genomics Viewer from Broad Institute (software.broadinstitute.org/software/igv/).

i. RNA Sequencing and Analysis

For samples from the MCC tumors and newly generated cell lines, RNA was first assessed for quality using the Agilent Bioanalyzer (DV200 metric). 100 ng of RNA were used as the input for first strand cDNA synthesis using Superscript III reverse transcriptase and Illumina's TruSeq RNA Access Sample Prep Kit. Synthesis of the second strand of cDNA was followed by indexed adapter ligation with UMI (unique molecular index) adaptors. Subsequent PCR amplification enriched for adapted fragments. Amplified libraries were quantified, normalized, pooled, and hybridized with exome targeting oligos. Following hybridization, bead clean-up, elution, and PCR was performed to prepare library pools for sequencing on Illumina flowcell lanes. Transcriptomes were sequenced to a coverage of at least 50 million reads in pairs.

For fibroblast and keratinocyte control lines, raw FASTQ files were downloaded from the Sequence Read Archive using R Bioconductor package SRAdb with accession codes SRP126422 (4 replicates from control samples ‘NN’) and SRP131347 (6 replicates with condition: control and genotype: control). Raw FASTQ files for MKL-1 and WaGa were obtained from the control shScr MKL-1 and WaGa cell lines that are described below (Methods: MKL-1 shMYCL and WaGa shST/LT line generation and sequencing). FASTQ files from fibroblasts, keratinocytes, MKL-1, and WaGa were then aligned using STAR version 2.7.3a, using the index genome reference file downloaded from ftp://ftp.ebi.ac.uk/pub/databases/gencode/Gencode_human/release_19/GRCh37.p13.genome.fa.gz, the transcript annotation file downloaded from https://data.broadinstitute.org/snowman/hg19/star/gencode.v19.annotation.gtf, and with the following options: --twopassMode Basic, --outSAMstrandField intronMotif, --alignIntronMax 1000000, --alignMatesGapMax 1000000, --sjdbScore 2, --outSAMtype BAM Unsorted, --outSAMattributes NH HI NM MD AS XS, --outFilterType BySJout, --outSAMunmapped Within, --genomeLoad NoSharedMemory, --outFilterScoreMinOverLread 0, --outFilterMatchNminOverLread 0, --outFilterMismatchNmax 999, and outFilterMultimapNmax 20. Duplicates were marked with picard MarkDuplicates version 2.22.0-SNAPSHOT.

RNA-sequencing BAM files for MCC tumor and cell line samples were downloaded from the Broad Genomics Firecloud/Terra platform using the Google Cloud Storage command line tool gsutil version 4.5 (github.com/GoogleCloudPlatform/gsutil/).

Gene counts were obtained from BAM files using featureCounts version 2.0.0. Very lowly expressed genes with average count across samples less than 1 were excluded from analysis. Between-sample distance metrics (FIG. 1G) were computed using the Euclidean distance on the vectors of variance-stabilized counts obtained from the vst function in the DESeq2 R Bioconductor package.

Differential expression analysis was carried out between IFN-γ plus and minus samples (adjusting for viral status as a covariate) using the negative binomial GLM Wald test of DESeq2, where significance was assessed using the p-values adjusted for multiple comparisons under default settings. To account for potential global gene expression differences among sample groups, RUVg was used to estimate latent factors of unwanted variation from the list of housekeeping genes downloaded from www.tau.ac.il/˜elieis/HKG/HK_genes.txt. The largest factor of unwanted variation was then used as a covariate in the DESeq2 models to adjust for latent variation unrelated to library size. The normalized counts adjusted for the latent factors of variation returned by RUVg were visualized in FIG. 2A.

j. MCPyV Viral DNA and RNA Detection

DNA detection of MCPyV in MCC tumor samples was performed with ViroPanel. For viral transcript quantification of RNA-seq, the Merkel Cell Polyomavirus reference sequence was downloaded from www.ebi.ac.uk/ena/data/view/EU375804&display=fasta. Reads that did not map to the human reference sequence were extracted from RNA-seq and ViroPanel BAM files of tumor and cell line using SAMtools view version 1.10 and realigned to a modified Merkel Cell Polyomavirus reference sequence (HM355825.1, recircularized such that the reference sequence ends when the VP2 coding sequence ends) using BWA version 0.7.17-r1188. Coverage at each position was assessed with samtools using the command ‘samtools depth-aa-d0’, and coverage depth was plotting in R version 3.5.1 using the ggplot2 and gggenes packages.

k. Single-Cell RNA Sequencing

Tumor samples from MCC-336 (MCPyV+) and MCC-350 (MCPyV−) were processed for single cell RNA-seq (scRNAseq). Cells were thawed and washed twice in RPMI and 10% FBS before undergoing dead cell depletion (Miltenyi 130-090-101). Viable MCC tumor cells were resuspended in PBS with 0.04% BSA at the cell concentration of 1,000 cells/μL. 17,000 cells were loaded onto a 10× Genomics Chromium™ instrument (10× Genomics) according to the manufacturer's instructions. The scRNAseq libraries were processed using Chromium™ single cell 5′ library & gel bead kit (10× Genomics). Quality control for amplified cDNA libraries and final sequencing libraries were performed using Bioanalyzer High Sensitivity DNA Kit (Agilent). ScRNAseq libraries were normalized to 4 nM concentration and pooled, and then the pooled libraries were sequenced on Illumina NovaSeq S4 platform. The sequencing parameters were: Read 1 of 150 bp, Read 2 of 150 bp, and Index 1 of 8 bp. Reads from both samples were demultiplexed and aligned to hg19 using Cell Ranger (v. 3.0.2) and the transcript quantities were co-analyzed using the Seurat (v. 3.1.5) R package. Only cells expressing >1,500 and <7,500 genes and <10% mitochondrial genes were kept for further analysis, leaving a total of 15,808 cells sequenced to a mean depth of 4,231.9 genes/cell. The data were normalized and the top 2,000 variable features were identified. Subsequently, the data were scaled while regressing out variation from gene count, mitochondrial percentage, and cell cycle stage. This was followed by principal component analysis, batch correction using Harmony (v. 1.0)⁸¹, UMAP analysis, and finally, Louvain clustering at resolution=0.3. The immune cell cluster was identified by the expression of CD45 (PTPRC) and MCC clusters were identified by expression of ATOH1, SYP, and SOX2.

l. Immunoprecipitation, Mass Spectrometry Analysis, and Peptide Identification

Up to 40 million or 0.2 g of MCC cells were immunoprecipitated. Briefly, MCC cells were harvested and lysed in ice-cold lysis buffer containing 40M Tris (pH 8.0), 1 mM EDTA (pH 8.0), 0.1M sodium chloride, Triton X-100, 0.06M octyl β-d-glucopyranoside, 100 U/mL DNAse I, 1 mM phenylmethanesulfonyl fluoride (all from Sigma Aldrich), and protease inhibitor cocktail (Roche Diagnostics). Cell lysate was centrifuged at 12,700 rpm at 4° C. for 22 min. Lysate supernatant was coupled with Gammabind Plus sepharose beads (GE Healthcare) and incubated with 10 μg of HLA-I antibody (Clone W6/32, Santa Cruz Biotechnologies) at 4° C. under rotary agitation for 3 h. After incubation, the lysate-bead-antibody mixture was briefly centrifuged and the supernatant was discarded. Beads were washed with lysis buffer, consisting of wash buffer containing 40 mM Tris (pH 8.0), 1 mM EDTA (pH 8.0), 0.1M sodium chloride, 0.06M octyl β-d-glucopyranoside, and 20 mM Tris buffer, without protease inhibitors. Gel loading tips (Fisherbrand) were used to remove as much fluid from beads as possible. Peptides of up to three immunoprecipitations were combined, acid eluted, and analyzed using LC/MS-MS. Briefly, peptides were resuspended in 3% acetonitrile with 5% formic acid and loaded onto an analytical column (20-30 cm with 1.9 μm C18 Reprosil beads, Dr. Maisch HPLC GmbH); packed in-house). Peptides were eluted in a 6-30% gradient (EasyLC 1000 or 1200, Thermo Fisher Scientific) and analyzed on a QExactive Plus, Fusion Lumos, or Orbitrap Exploris 480 (Thermo Fisher Scientific). For Lumos measurements, peptides were also subjected to fragmentation if they were singly charged. For Orbitrap Exploris measurements (2 immunoprecipitations pooled, +/−IFN-γ, FIG. 3) and detection of the large T antigen peptide (3 immunoprecipitations of the MCC-367 cell line treated with IFN-γ) peptides were further fractionated using stage tip basic reverse phase separation with 2 punches of SDB-XC material (Empore 3M) and increasing concentrations of acetonitrile (5%, 10% and 30% in 0.1% NH4OH, pH 10). Fractions were analyzed on a Fusion Lumos or Orbitrap Exploris 480 equipped with a FAIMSpro interface.

Immunopeptidomes of USP7 inhibitor treated cell lines were eluted as described above, followed by labeling with TMT6 reagent (Thermo Fisher; 126-USP7iA, 127-WT, 128 USP7iA, 129 WT, 130-USP7iB, 131 USP7iB) and then pooled for subsequent fractionation using basic reversed phase fractionation with increasing concentrations of acetonitrile (10%, 15% and 50%) in 5 mM ammonium formate (pH 10) and analysis on an Orbitrap Exploris 480 with FAIMSpro. Data acquisition parameters were as above with NCE set to 34 and 2 second dynamic exclusion.

Mass spectra were interpreted using Spectrum Mill software package v7.1 pre-Release (Broad Institute, Cambridge, Mass.). MS/MS spectra were excluded from searching if they did not have a precursor MH+ in the range of 600-4000, had a precursor charge >5, or had a minimum of <5 detected peaks. Merging of similar spectra with the same precursor m/z acquired in the same chromatographic peak was disabled. MS/MS spectra were searched against a protein sequence database that contained 90,904 entries, including all UCSC Genome Browser genes with hg19 annotation of the genome and its protein coding transcripts (52,788 entries), common human virus sequences (30,181 entries), recurrently mutated proteins observed in tumors from 26 tissues (4,595 entries), 264 common laboratory contaminants as well as protein sequences containing somatic mutations detected in MCC cell lines (3,076 entries). MS/MS search parameters included: no-enzyme specificity; ESI-QEXACTIVE-HCD-HLA-v3 instrument scoring; fixed modification: cysteinylation of cysteine; variable modifications: oxidation of methionine, carbamidomethylation of cysteine and pyroglutamic acid at peptide N-terminal glutamine; precursor mass tolerance of ±10 ppm; product mass tolerance of 10 ppm, and a minimum matched peak intensity of 30%. Peptide spectrum matches (PSMs) for individual spectra were automatically designated as confidently assigned using the Spectrum Mill auto-validation module to apply target-decoy based FDR estimation at the PSM level of <1% FDR. Peptide auto-validation was done separately for each sample with an auto thresholds strategy to optimize score and delta Rank1-Rank2 score thresholds separately for each precursor charge state (1 through 4) across all LC-MS/MS runs per sample. Score threshold determination also required that peptides had a minimum sequence length of 7, and PSMs had a minimum backbone cleavage score of 5. Peptide and PSM exports were filtered for contaminants including potential carry over tryptic peptides and peptides identified in a blank bead sample. For TMT-labeled samples, peptides derived from keratin proteins were removed and TMT intensity values were normalized to the global median. P-values were calculated using in house software based on the limma package in R.

m. Whole Proteome Analysis and Interpretation

Protein expression of MCC cell lines was assessed. Briefly, cell pellets of MCC cell lines with and without IFN-γ treatment were lysed in 8M Urea and digested to peptides using LysC and Trypsin (Promega). 400 μg peptides were labeled with TMT10 reagents (Thermo Fisher, 126-MCC-290, 127N-MCC-350_IFN, 127C MCC-275_IFN, 128N MCC-275, 128C MCC-350, 129N_MCC-301_IFN, 129C-MCC-277_IFN, 130N-MCC-290_IFNy, 130C MCC-277, 131 MCC-301) and then pooled for subsequent fractionation and analysis. Pooled peptides were separated into 24 fractions using offline high pH reversed phase fractionation. 1 μg per fraction was loaded onto an analytical column (20-30 cm with 1.9 μm C18 Reprosil beads [Dr. Maisch HPLC GmbH], packed in-house, PicoFrit 75 μM inner diameter, 10 μM emitter [New Objective]). Peptides were eluted with a linear gradient (EasyNanoLC 1000 or 1200, Thermo Scientific) ranging from 6-30% Buffer B (either 0.1% formic acid or 0.5% AcOH and 80% or 90% acetonitrile) over 84 min 30-90% Buffer B over 9 min, and held at 90% Buffer B for 5 min at 200 nl/min. During data dependent acquisition, peptides were analyzed on a Fusion Lumos (Thermo Scientific). Full scan MS was acquired at a 60,000 from 300-1,800 m/z. AGC target was set to 4e5 and 50 ms. The top 20 precursors per cycle were subjected to HCD fragmentation at 60,000 resolution with an isolation width of 0.7 m/z, 34 NCE, 3e4 AGC target, and 50 ms max injection time. Dynamic exclusion was enabled with a duration of 45 sec.

Spectra were searched using Spectrum Mill against the database described above excluding MCC variants, specifying Trypsin/allow P (allows K—P and R—P cleavage) as digestion enzyme and allowing 4 missed cleavages, and ESI-QEXACTIVE-HCD-v3. Carbamidomethylation of cysteine was set as a fixed modification. TMT labeling was required at lysine, but peptide N-termini were allowed to be either labeled or unlabeled. Variable modifications searched include acetylation at the protein N-terminus, oxidized methionine, pyroglutamic acid, deamidated asparagine, and pyrocarbamidomethyl cysteine. Match tolerances were set to 20 ppm on MS1 and MS2 level. PSMs score thresholding used the Spectrum Mill auto-validation module to apply target-decoy based FDR in 2 steps: at the peptide spectrum match (PSM) level and the protein level. In step 1 PSM-level auto-validation was done first using an auto-thresholds strategy with a minimum sequence length of 8; automatic variable range precursor mass filtering; and score and delta Rank1-Rank2 score thresholds optimized to yield a PSM-level FDR estimate for precursor charges 2 through 4 of <1.0% for each precursor charge state in each LC-MS/MS run. To achieve reasonable statistics for precursor charges 5-6, thresholds were optimized to yield a PSM-level FDR estimate of <0.5% across all LC runs per experiment (instead of per each run), since many fewer spectra are generated for the higher charge states. In step 2, protein-polishing auto-validation was applied to each experiment to further filter the PSMs using a target protein-level FDR threshold of zero, the protein grouping method expand subgroups, top uses shared (SGT) with an absolute minimum protein score of 9. TMT10 reporter ion intensities were corrected for isotopic impurities in the Spectrum Mill protein/peptide summary module using the afRICA correction method which implements determinant calculations according to Cramer's Rule and correction factors obtained from the reagent manufacturer's certificate of analysis (www.thermofisher.com/order/catalog/product/90406) for lot number TB266293.

n. ELISpot

Matching patient peripheral blood mononuclear cells (PBMCs) from patient MCC-367 were thawed, and 10⁷ cells per well were seeded in 24 well plates overnight. Cells were stimulated with 10 μg/ml of the LT antigen peptide TSDKAIELY (identified in the MCC-367 HLA peptidome, FIG. 3F) in complete DMEM supplemented with 10% Human serum and 20 ng/ml IL-7 (PeproTech). After 3 days of stimulation, cells were supplemented with 20 units/mL IL-2 (PeproTech). After 10 days of stimulation, cells were cytokine deprived overnight. 50,000 cells per well were stimulated in an IFN-γ ELISpot assay with 10 μg/ml of the TSDKAIELY peptide. DMSO and an HIV-GAG peptide were used as negative controls. CEF (Mabtech) and PHA (Sigma Aldrich) were used as positive controls (not shown). ELISpot and T cell culture methods were described in detail previously.

o. ORF Screen

The human ORFeome version 8.1 lentiviral library, which contains 16,172 unique ORFs mapping to 13,833 genes, was supplied as a gift from the Broad Genetic Perturbations Platform. 75 million MCC-301 cells were transduced with ORFeome lentivirus to achieve an infection rate of approximately 30-40%. Two days later, transduced cells were selected with three days of 0.5 μg/mL puromycin (Santa Cruz Biotechnology #SC-10871) treatment. Between 7-10 days after transduction, cells were stained with an anti-HLA-ABC-PE antibody (W6/32 clone, Biolegend #311405) and sorted on a BD FACSAria II, gating for the top and bottom 10% of HLA-ABC-PE staining. Sorted cells were washed with PBS, flash frozen, and stored at −80° C. Subsequently, genomic DNA containing stably integrated ORF sequences was isolated from the sorted cell pellets. The screen was performed in triplicate. Isolated genomic DNA was then used as a template for indexed PCR amplification of the construct barcode region. Pooled PCR products were purified and run on an Illumina HiSeq.

p. CRISPR-KO Screen

The Brunello human CRISPR knockout pooled plasmid library (1-vector system) was a gift from David Root and John Doench (Addgene #73179). 50 ng of the Brunello plasmid library was electroporated into ElectroMAX Stbl4 competent cells (ThermoFisher #11635018) and incubated overnight at 30° C. on 24.5×24.5 cm agar bioassay plates. 20 hours later, colonies were harvested and pooled, and the amplified plasmid DNA (pDNA) was extracted and purified. To confirm that library diversity was maintained after amplification, sgRNA barcode construct regions were PCR amplified in pre- and post-amplification library aliquots. PCR products were purified and sequenced on an Illumina MiSeq. Sequencing data from pre- and post-amplification aliquots were compared to ensure similar diversity. To produce lentivirus, HEK-293T cells were transfected with pDNA, VSV-G, and psPAX2 plasmids using the TransIT-LT1 transfection reagent (Mirus #MIR2300). Lentivirus was harvested 48 hours post-transfection and flash frozen. To titrate lentivirus, 1.5 million cells MCC-301 cells were transduced with 100, 200, 300, 500, and 700 μL of virus. From each condition, half of the cells were selected with 0.5 μg/mL puromycin (Santa Cruz Biotechnology #SC-10871) while the other half were left untreated. Infection rates were calculated by comparing live cell counts in selected and unselected conditions.

Lentiviral transduction and FACS screening were performed in triplicate analogously to the ORF screen with the following exceptions: 150 million MCC-301 cells were transduced per replicate, and cells were sorted 10-14 days after transduction. Additionally, a representative pellet (40 million cells) after transduction but before flow cytometry selection was harvested and sequenced from all three replicates to assess sgRNA representation (FIG. 4F).

q. Screen Data Analysis

Unprocessed FASTQ reads were converted to log 2-normalized scores for each construct using PoolQ v2.2.0 (portals.broadinstitute.org/gpp/public/software/poolq). For each of the three replicates, log₂-fold changes (LFCs) between the normalized count scores of the HLA-I-high and HLA-I-low populations were calculated for each construct.

For the ORF screen, ORF constructs were then ranked based on their median LFC values, and corresponding p values were calculated using a hypergeometric distribution model (portals.broadinstitute.org/gpp/public/analysis-tools/crispr-gene-scoring). In cases where there were multiple ORFs mapping to one gene, LFC values were averaged across all constructs to generate a gene-level value. Sample quality for each sorted population was assessed by calculating log-normalized ORF construct scores (log₂ (ORF construct reads/total reads×10⁶+1) and confirming that the mean construct frequency was no less than 10% of the expected frequency if all constructs were equally represented (corresponding to mean log-normalized score cutoff of 2.84) (FIG. 4F (left)).

For the CRISPR screen, using equivalent cutoff criteria as above corresponding log-normalized score cutoff of 3.80), replicate 2 was discarded because the mean log-normalized score of the replicate 2 HLA-I-high sorted population was only 0.413 (FIG. 4F (right)). Subsequently, LFC values for each sgRNA were averaged between replicate 1 and 3 only and then input into the STARS software (portals.broadinstitute.org/gpp/public/analysis-tools/crispr-gene-scoring), which employs a binomial distribution model to rank genes based on the ranks of their corresponding individual sgRNAs.

For GSEA analysis, ranked ORF and CRISPR lists were generated by averaging the LFC values of all constructs mapping to or targeting a particular gene and ranking genes based on this average LFC. These ranked lists were then used as input for GSEAPreranked (enrichment statistic—weighted; max gene set size—500; min gene set size—15).

r. Generation of ORF Lines

Single ORF constructs cloned into the pLX_TRC317 plasmid were a gift from the Broad Institute Genetic Perturbation Platform (portals.broadinstitute.org/gpp/public/). ORF plasmids, psPAX2, and VSV-G were transfected into HEK-293T cells to produce lentivirus. MCC-301 and MCC-277 cells were transduced with individual ORF lentivirus in 2 μg/mL polybrene, and spinfection was performed at 2,000 rpm for 2 hours at 30° C. Two days after transduction, transduced cells were selected with three days of 0.5 μg/mL puromycin treatment. Flow cytometry was performed as described above (see Methods: Flow cytometry) using either a PE-conjugated HLA-ABC (W6/32) antibody (BioLegend #311406) for MCC-301 lines or a AF647-conjugated HLA-ABC (W6/32) antibody (Santa Cruz Biotechnology #sc24637) for MCC-277 lines.

s. Generation of CRISPR KO Lines

Forward and reverse oligos with the sequence 5′ CACCG----sgRNA sequence---3′ and 5′ AAAC---reverse complement of sgRNA---C 3′ were synthesized by Eton Biosciences. Forward and reverse oligos were annealed and phosphorylated, producing BsmBI-compatible overhangs. LentiCRISPRv2 vector (Addgene #52961) was digested with BsmBI, dephosphorylated with shrimp alkaline phosphatase, and gel purified. Vector and insert were ligated at a 1:8 ratio with T7 DNA ligase at room temperature and transformed into Stbl3 chemically competent cells (ThermoFisher #C737303). Correct sgRNA cloning was confirmed via Sanger sequencing using the following primer: 5′-GATACAAGGCTGTTAGAGAGATAATT-3′. Lentivirus was produced in HEK-293T cells (psPAX2, VSV-G, and cloned CRISPR plasmid), and MCC-301 cells were transduced with single construct lentivirus for single knockout lines, or with two lentivirus pools containing two different sgRNAs against the same gene for double knockout lines. Transduction was performed in the same manner as for the CRISPR-KO library. To validate gene editing for the single knockout lines, genomic DNA was extracted from both single knockout lines and WT MCC-301. Genomic DNA was then used as a template for PCR, with primers designed to flank the putative sgRNA binding sites. PCR products were purified and Sanger sequenced at Eton Biosciences. The percent of edited cells was then determined by TIDE⁴⁹ using WT MCC-301 as a reference. Flow cytometry was performed as described above (see Methods: Flow cytometry) using either a PE-conjugated HLA-ABC (W6/32) antibody (BioLegend #311406) for single knockout lines or a AF647-conjugated HLA-ABC (W6/32) antibody (Santa Cruz Biotechnology #sc24637) for double knockout lines.

t. Western Blot Analysis

Briefly, 1 million MCC-301 cells were transduced with single lentiviral constructs against a non-targeting control, PCGF1, BCORL1 or USP7. Two days after transduction, cells were subjected to selection with 0.5 ug/mL puromycin treatment for three days. For IFN-γ treatments, MCC-301 cell lines were treated with indicated doses of IFN-γ for 24 hours before harvesting for Western Blot analysis. Cells were collected by centrifugation, washed in PBS and lysed in EBC buffer (50 mM Tris-HCl, 200 mM NaCl, 0.5% NP-40, 0.5 mM EDTA) supplemented with protease and phosphatase inhibitors (Millipore) and 2-Mercaptoethanol (Bio-Rad) to obtain whole cell extracts. The cell extracts were clarified by centrifugation. The protein content of each sample was determined using BioRad BradFord assay following the addition of 6× Laemmli buffer (Boston bioproducts) and boiling of the samples at 95° C. for 5 minutes. A 4-20% gradient gel (Bio-Rad) was run for the analysis and the proteins were transferred to a 0.2 μm Nitrocellulose membrane (Bio-Rad). The membrane was blocked using 5% milk in TBST at Room temperature for 1 hour followed by incubation with appropriate primary antibodies [USP7 (Life Technologies #PA534911), PCGF1 (E8, Santa Cruz Biotechnology #SC-515371), TAP1 (Cell Signaling Technology #12341S), TAP2 (Cell Signaling Technology #12259S), p53 (Santa Cruz Biotechnology #SC-126), pan-MYC (Abcam #ab195207), Vinculin (Sigma #V9131), TBP (Cell Signaling Technology #8515S)] diluted according to manufacturer's specifications in 5% milk in TBST at 4° C. overnight. The next day, membranes were washed thrice with TBST and incubated with the appropriate secondary antibody (Bethyl, Goat anti-mouse #A90-116P or Goat anti-Rabbit #A120-101P) diluted in 1% milk in TBST for one hour at room temperature. The membrane was washed thrice with TBST and incubated briefly with Immobilon Western Chemiluminescent (Millipore) HRP substrate followed by visualization of the signal on the G-box imaging system (Syngene). Raw Western Blot images were processed for visualization using the ImageJ software.

u. MKL-1 shMYCL and WaGa shST/LT RNA-Seq and Flow Cytometry

A scramble shRNA constitutively expressed from the lentiviral PLKO vector (shScr) has been reported before (Addgene #1864). The MYCL and EP400 shRNA target sequences were designed using Block-iT RNAi Designer (Life Technologies). MYCL target—GACCAAGAGGAAGAATCACAA; shEP400-2 target—GCTGCGAAGAAGCTCGTTAGA, shEP400-3 target—GGAGCAGCTTACACCAATTGA. Annealed forward and reverse oligos of shScr, shMYCL, shEP400-2, and shEP400-3 ( ) were cloned between AgeI/EcoRI sites of the doxycycline inducible shRNA vector Tet-pLKO-puro (a gift from Dmitri Wiederschain, Addgene #21915). 293T cells were transfected with the Tet-PLKO-puro plasmids plus psPAX2 packaging and VSV-G envelope plasmids (Addgene #12260 and #12259) to generate lentiviral particles for MKL-1 cell transduction. Transduced MKL-1 cells were selected with 1 μg puromycin for 4 days to generate Dox-inducible MKL-1 shScr, shMYCL, shEP400-2, and shEP400-3 lines. The Dox-inducible WaGa shST/LT line was a gift from Roland Houben.

For RNA-seq, cells were treated with dox as follows: MKL-1 shMYCL and shScr-2 days Dox, MKL-1 shEP400-2, -3 and shScr-6 days Dox, WaGa shST/LT cells with or without Dox-6 days. Total RNA was extracted using RNeasy Plus Mini Kit (Qiagen). mRNA was isolated with NEB-Next Poly(A) mRNA Magnetic Isolation Module (New England BioLabs). Sequencing libraries were prepared with NEBNext mRNA library Prep Master Mix Set for Illumina (New England BioLabs) and passed Qubit, Bioanalyzer, and qPCR QC analyses. 50 cycles single-end sequencing was performed on the Illumina HiSeq 2000 system. Reads were mapped to the hg19 genome by TOPHAT. HTSeq was used to create a count file containing gene names. The R package DESeq2 was used to normalize counts and calculate total reads per million (TPM) and determine differential gene expression. Quality control was performed by inspecting a MA plot of differentially expressed genes. RNA-seq data are available from the Gene Expression Omnibus with accession number GSE69878. For GSEA analysis, genes were ranked based on their LFC value from DESeq2. These ranked lists were then used as input for GSEAPreranked (enrichment statistic—weighted; max gene set size—500; min gene set size—15).

For flow cytometry, shMYCL and shScr MKL-1 cells were treated with 0.2 μg/mL doxycycline for 7 days, refreshing with doxycycline-containing media every 3 days. In addition, shMYCL cells containing a constitutively expressed (Addgene, #17486) shRNA-resistant MYCL (shMYCL+MYCL) construct were identically treated. Single cell suspensions were prepared non-enzymatically via treatment with Versene (Gibco 15040066). Cells were incubated with Human True-Stain FcX (BioLegend #422302), followed by staining with an anti-HLA-A/B/C antibody (SCBT, #32235) or isotype-matched IgG control (SCBT, #24637) conjugated to Alexa Fluor 647. Stained cells were strained through a 100 m filter and fluorescence was measured via flow cytometry (BD, LSR Fortessa). Single cells were selected utilizing FSC-H/FSC-A discrimination and the geometric mean of Alexa Fluor 647 fluorescence was calculated from the single cell population.

Oligos

| Oligo Name | Sequence | Notes |
|---|---|---|
| BCORL1-1 fwd | CACCGTCCCGCATCTGACAGCGCCG | Oligo for guide RNA cloning |
| BCORL1-1 rev | AAACCGGCGCTGTCAGATGCGGGAC | Oligo for guide RNA cloning |
| BCORL1-2 fwd | CACCGGGAGGCGGGATATATACCAG | Oligo for guide RNA cloning |
| BCORL1-2 rev | AAACCTGGTATATATCCCGCCTCCC | Oligo for guide RNA cloning |
| USP7-1 fwd | CACCGTTGATGACGACGTGGTGTCA | Oligo for guide RNA cloning |
| USP7-1 rev | AAACTGACACCACGTCGTCATCAAC | Oligo for guide RNA cloning |
| USP7-2 fwd | CACCGGGCAGTAGAACAGCTCGATG | Oligo for guide RNA cloning |
| USP7-2 rev | AAACCATCGAGCTGTTCTACTGCCC | Oligo for guide RNA cloning |
| PCGF1-1 fwd | CACCGCCACGAAGTAGCCGGCGCAT | Oligo for guide RNA cloning |
| PCGF1-1 rev | AAACATGCGCCGGCTACTTCGTGGC | Oligo for guide RNA cloning |
| PCGF1-2 fwd | CACCGGCTCATCATAGCGATAGTAG | Oligo for guide RNA cloning |
| PCGF1-2 rev | AAACCTACTATCGCTATGATGAGCC | Oligo for guide RNA cloning |
| CTRL-1 fwd | CACCGTGCGGCGTAATGCTTGAAAG | Oligo for guide RNA cloning |
| CTRL-1 rev | AAACCTTTCAAGCATTACGCCGCAC | Oligo for guide RNA cloning |
| CTRL-2 fwd | CACCGGGATTAATTCGCTAAATGAT | Oligo for guide RNA cloning |
| CTRL-2 rev | AAACATCATTTAGCGAATTAATCCC | Oligo for guide RNA cloning |
| shMYCL fwd | CCGGACCAAGAGGAAGAATCACAATCAAGA | Oligo for shRNA |
| | GTTGTGATTCTTCCTCTTGGTCTTTTT | |
| shMYCL rev | AATTAAAAAGACCAAGAGGAAGAATCACA | Oligo for shRNA |
| | ACTCTTGATTGTGATTCTTCCTCTTGG | |
| shEP400-2 fwd | ccggCTGCGAAGAAGCTCGTTAGATCAAGAG | Oligo for shRNA |
| | TCTAACGAGCTTCTTCGCAGCttttt | |
| shEP400-2 rev | aattAAAAAGCTGCGAAGAAGCTCGTTAGACT | Oligo for shRNA |
| | CTTGATCTAACGAGCTTCTTCGCAG | |
| shEP400-3 fwd | ccggAGCAGCTTACACCAATTGAtcaagagTCAA | Oligo for shRNA |
| | TTGGTGTAAGCTGCTCCttttt | |
| shEP400-3 rev | aattAAAAAGGAGCAGCTTACACCAATTGACT | Oligo for shRNA |
| | CTTGATCAATTGGTGTAAGCTGCT | |
| LentiCRISPRv2 | GATACAAGGCTGTTAGAGAGATAATT | N/A |
| sequencing | | |
| primer | | |
| USP7 ChIP fwd | CCAACGACCAACTCCCTAAAT | ChIP-qPCR primer |
| USP7 ChIP rev | AAGGCACTGTAGTTTGAGGTATAG | ChIP-qPCR primer |
| PCGF1 ChIP fwd | TCGCCTCCTTCATCACACTA | ChIP-qPCR primer |
| PCGF1 ChIP rev | CGAGTCCACGTGAGGGAA | ChIP-qPCR primer |
| Intergenic ChIP | CTTCTTCCTTCCGGCTTTCT | ChIP-qPCR primer |
| fwd | | |
| Intergenic ChIP | AGCTGGGAGAGGACACACAC | ChIP-qPCR primer |
| rev | | |

v. ChIP-Seq and ChIP-qPCR

ChIP-seq data for MAX, EP400, ST, H3K4me3, and H3K27ac was generated. For ChIP-qPCR, the following primers were designed using PrimerQuest (IdtDNA) based on ChIP-seq data displayed in UCSC genome browser ( ). qPCR was performed using the Brilliant III ultra-fast SYBR green qPCR master mix (Agilent) on the AriaMx Real-time PCR System (Agilent) by following the instruction manual.

w. MCC Tumor RNA-Seq Cohort

Tumor biopsies were collected from 52 patients at the DFCI and preserved for RNA isolation via addition of RNAlater (Sigma-Aldrich). Preserved tissue was homogenized via TissueRuptor (QIAGEN) and RNA was harvested via AllPrep DNA/RNA Mini Kit (QIAGEN). RNA was submitted for library construction utilizing the NEBNext Ultra II RNA Library Prep Kit for Illumina (NEB). Paired-end sequencing was performed on the NovaSeq 6000 system for 150 cycles in each direction (Novogene). Raw paired-end sequencing data were broadly assessed for quality via FastQC (www.bioinformatics.babraham.ac.uk/projects/fastqc/). Samples passing quality control were quantified to the transcript level via Salmon utilizing Ensembl gene annotations for the GRCh38.p13 genome assembly. Normalized gene-level counts were prepared with TxImport and DESeq2. To identify virus-positive or virus-negative samples, paired-end reads were mapped to the MCPyV genome (R17b isolate) via BWA and those sample containing MCPyV-specific reads (>100) were considered virus-positive. For the RNA-seq heatmap, z-scores of the log 2-normalized gene-level counts were calculated. One tumor sample was subsequently discarded as an outlier because the z-score was >3.5 or <−3.5 in 7 of the 18 genes analyzed in this sample (for comparison, the range of z-scores for all 18 genes in all other samples was −3.45 to 2.47). The remaining 51 tumor samples were subsequently clustered by Euclidian distance to generate the RNA-seq heatmap. Tumor purity was determined using the ESTIMATE R Package. Tumor purity percentage was calculated from the ESTIMATE score using the equation: cos(0.6049872018+0.0001467884×ESTIMATE score) as published.

x. PCGF1-KO RNA-Seq and Western Blots

RNA was extracted from three technical replicates of the MCC-301 PCGF1-KO #2 line (second-highest scoring guide RNA) and of an MCC-301 line transduced with a non-targeting sgRNA control and Cas9. Sample preparation and sequencing was performed as described above in “RNA sequencing and analysis”. Subsequently, raw FASTQ files were broadly assessed for sequencing quality via FastQC (Babraham Institute), with those of passing quality used for further analysis. Salmon was used to map raw reads to the decoy-aware transcriptome of GRCh38p.13 v99 (Ensembl) with the following stipulations: --writeUnmappedNames, --seqBias, --gcBias, --validateMappings. Raw transcript-level counts were converted to gene-level counts via TxImport and differential gene expression analysis was performed using DeSeq2.

For TAP1 Western blots, IFN-γ titration was first performed in MKL-1 cells (FIG. 4Q) to determine the IFN-γ range over which TAP1 expression became detectable. Concentrations of 0, 100, and 1,000 U/mL IFN-γ were subsequently used for TAP1 Western blots in MCC-301 PCGF1-KO and control sgRNA lines.

y. Cell Cycle Analysis

1 million MKL-1 control or p53 KO cells were plated and treated with DMSO, XL177A (100 nM) or XL177B (100 nM) for three days. During the last hour of the three-day treatment, the cells were pulsed with 10 μM EdU nucleotide. The cells were collected by centrifugation, treated with Accutase™ (Stem Cell Technologies) to break apart clumps, washed with PBS and fixed using 4% Formaldehyde solution in PBS at Room temperature for 15 mins. Cells were washed with 1% BSA in PBS and resuspended in 70% ice cold ethanol and incubated at −20° C. overnight for additional fixing and permeabilization. The cells were stored in 70% ethanol at −20° C. until the day the data was acquired. On the day of data acquisition, the cells were collected by centrifugation and washed twice with PBS. The incorporated EdU in the cells were labeled with a CLICK reaction cocktail (1 mM CuSO4, 100 μM THPTA, 100 mM sodium ascorbate, and 2.2 μM Alexa 647 azide in PBS) at room temperature with rocking for 30 minutes. The samples were then washed with 1% BSA in PBS once followed by two washes with PBS and incubated with a 1 μg/ml DAPI, 100 ng/ml RNase A solution for one hour at Room temperature to stain the DNA. The samples were then passed through strainer tubes and analyzed using a BD Fortessa analyzer. The flow cytometry data was analyzed using the FlowJo Software. The percentage of cells in each cell cycle phase was represented using GraphPad PRISM software.

z. USP7 Inhibitor Experiments

For MCC-301 USP7 inhibitor experiments, two and a half million MCC cells were plated in a T25 flask and incubated with the USP7 inhibitor XL177A and control enantiomer XL177B at 10 μM, 1 μM, 100 nM, and 10 nM. Cells were incubated for 3 to 4 days. Post incubation, one million cells were treated with Versene (Gibco) to dissociate cell clusters. Surface Fc receptors were blocked with 5 μL Human TruStain FcX (Biolegend #422302). Surface HLA-I was stained with 5 μL of Pan HLA-Class I antibody (Clone W6/32, Santa Cruz Biotechnologies) for 30 minutes in dark at 4° C. Cells were washed with PBS and fixed with 4% paraformaldehyde fixation buffer (Biolegend). Cells were analyzed on a BD LSRFortessa. MCC-301 data are representative of 4 independent experiments. To perform statistical analysis, for each cell line, one-way ANOVA was first performed on the MFIs of the DMSO group and all experimental groups. Then, individual Welch t-tests were performed for each concentration, comparing the fold-changes of MFI (inhibitor)/mean MFI (DMSO control) between XL177A and XL177B.

For MKL-1 USP7 inhibitor experiments, p53-WT control lines (WT, scrambled, AAVS1) and three p53-KO lines were treated with USP7 inhibitors and assessed by flow cytometry for surface HLA I as described above for MCC-301. Because the root mean squared error differed considerably between the control lines and the p53-KO lines (12.2894 and 6.69844), the two groups were analyzed separately by two-way ANOVAs, and drug treatment was found to be a statistically significant source of variation in MFI in both cases (P=0.0003 in controls and P<0.0001 in p53-KO lines). ANOVA was followed by post hoc Tukey's multiple comparisons tests between XL177A, XL177B, and DMSO treatments to generate the p-values displayed in FIG. 6H.

aa. Dependency Map Correlations

The DepMap 20Q2 CRISPR dependency data were downloaded from www.depmap.org/portal/download. TP53 mutation status was assigned using the Cell-Line Selector tool on the DepMap Portal based on criteria of at least one coding mutation. Pearson coefficients were calculated using test.cor in R, and two-sided p-values outputted by this function were converted into FDR using p.adjust. Plots were generated using ggplot2, tidyverse, gridExtra, cowplot, and scales. GSEA was performed using a gene list ranked by −log(p-val) multiplied by (−1) if the Pearson correlation was negative.

### Quantification and Statistical Analysis

All flow cytometry bar graphs show mean fluorescence intensity of three technical or biological replicates, except for FIG. 1J and FIG. 1N which show one sample. Error bars indicates standard deviation, unless otherwise stated. P-value of 0.05 was used as the significance threshold in all experiments. Specific statistical tests used in each figure are mentioned in the figure legends and/or the methods section.

Specific software with version number, along with details of all statistical analyses are listed in the respective methods sections above. No randomization procedures or sample size calculations were carried out as part of the study. All analysis code including specific parameter settings for whole exome sequencing analysis, RNA-seq analysis, MCPyV viral transcript detection, and WGBS promoter signal extraction are made available in a GitHub repository under an MIT license at www.github.com/kdkorthauer/MCC. All analyses in R were carried out using version 3.6.2.

### Example 11: Reliable Generation of MCC Cell Lines from Primary Patient Samples

Since many established MCC lines have been multiply passaged in vitro and lack associated archival primary tumor material, a reliable approach to generate MCC lines was established. Although MCC is typically cultured in RPMI-1640 media, it was hypothesized that a neuronal stem cell media that was previously used to establish glioblastoma cell lines would facilitate cell line establishment, based on the neuroendocrine histology of MCC and a prior report of successful MCC line generation with a neural crest stem cell medium. Of 5 media formulations tested, NeuroCult NS-A Proliferation medium with growth factor supplementation consistently provided the highest in vitro growth rate, tripling cell numbers after seven days in culture (FIG. 1A) and facilitating reliable growth of multiple MCC tumor cell lines (FIG. 1P). Using this method, 11 stable cell lines from biopsies (n=4) or patient-derived xenograft (PDX) materials (n=7) (Table 6) were established. Consistent with established classical MCC lines, these lines grew mostly in tight clusters in suspension and stained positive for MCC markers SOX2 and CK20, except for CK20 negativity in MCC-320 (FIG. 1B; FIG. 1C). It was determined that 7 of the 11 lines (63.6%) were MCPyV+ using ViroPanel (FIG. 1D).

Whole-exome sequencing (WES) was performed on tumor DNA from 7 of 11 patients for whom matched cell line and germline DNA were available (Table 11). MCPyV− (n=2) and MCPyV+ (n=5) samples exhibited contrasting high (median 647 non-silent coding mutations per cell line, range 354-940) and low (median 40, range 18-73) TMBs (FIG. 1E; Table 11), respectively, as expected. The two analyzed MCPyV− lines contained mutations in RBI and TP53 (Table 11), consistent with previous studies. A median of 94.4% of cell line mutations was detected in the corresponding tumor or PDX samples (range 51-100%), and tumor-cell line pairs were associated most closely with each other based on mutational profiles (FIG. 1F). Of note, several PDX-derived tumor samples (Table 6) exhibited higher mutational burdens than their corresponding cell lines (FIG. 1E), likely due to variants associated with murine cell contamination. Corresponding RNA-sequencing (RNA-seq) of available matched tumors and cell line pairs (Table 1) detected MCPyV ST and LT antigen transcripts in all MCPyV+ samples (FIG. 1G; FIG. 1D). By unsupervised hierarchical clustering of these transcriptomes, each cell line associated most closely with its corresponding parent tumor (mean pairwise Spearman correlation 0.92) (FIG. 1G; FIG. 1H), rather than clustering by sample type, confirming that these cell lines faithfully recapitulate their parent tumors.

TABLE 11a
Summary of Omics Data

| | Tumor | | | Cell | Cell Line | | | | |
|---|---|---|---|---|---|---|---|---|---|
| | and | | | Line +/− | +/− IFN: | | | | Cell Line |
| | Cell | Cell | Tumor | IFN: | Full and | | | Tumor: | +/− IFN: |
| | Line | Line | RNA- | RNA- | Phospho- | ATAC- | | HLA | HLA |
| Patient | WES | WGS | seq | seq | Proteome | seq | WGBS | Peptidome | Peptidome |
| 277 | X | X | X | X | X | X | X | X | X |
| 282 | | X | X | X | | X | X | | |
| 290 | | X | X | X | X | X | X | X | X |
| 301 | X | X | X | X | X | X | X | | X |
| 320 | X | X | | X | | X | X | | |
| 336 | X | X | X | X | | X | X | | |
| 350 | X | X | X | X | X | X | X | | |
| 358 | | | X | X | | | | | |
| 367 | X | X | X | X | | X | X | | X |
| 383 | | | | X | | | | | |
| 2314 | X | X | X | X | | X | X | | |

TABLE 11b
HLA 1 and INF Mutations

| Start | End | Variant | Ref- | | Tumor_ | | | Trans | |
|---|---|---|---|---|---|---|---|---|---|
| Pos- | Pos- | Classi- | erence_ | TumorSeq_ | Seq_ | | HGVSp_ | cript_ | |
| ition | ition | fication | Allele | Allele1 | Allele1 | HGVSc | Short | ID | PolyPhen |
| 9981 | 9981 | Missense | T | T | C | c. | p. | ENST | possibly_ |
| 2397 | 2397 | Mutation | | | | 212A > G | D71G | 00000 | damaging |
| | | | | | | | | 50599 | (0.86) |
| | | | | | | | | 2 | |
| 9981 | 9981 | Missense | T | T | G | c. | p. | ENST | benign |
| 2415 | 2415 | Mutation | | | | 194A > C | K65T | 00000 | (0.009) |
| | | | | | | | | 50599 | |
| | | | | | | | | 2 | |
| 8662 | 8662 | Missense | A | A | T | c. | p. | ENST | probably_ |
| 559 | 559 | Mutation | | | | 922T > A | F308I | 00000 | damagin |
| | | | | | | | | 40215 | 8(0.942) |
| | | | | | | | | 7 | |
| 3260 | 3260 | Nonsense | G | G | A | c. | p. | ENST | |
| 9998 | 9998 | Mutation | | | | 581G > A | W194* | 00000 | |
| | | | | | | | | 34313 | |
| | | | | | | | | 9 | |
| 3248 | 3248 | Missense | T | T | A | c. | P- | ENST | benign |
| 0240 | 0240 | Mutation | | | | 1751A > T | Q584L | 00000 | (0.28) |
| | | | | | | | | 37988 | |
| | | | | | | | | 3 | |
| 5626 | 5626 | Missense | G | G | A | c. | p. | ENST | possibly_ |
| 619 | 619 | Mutation | | | | 656G > A | R219Q | 00000 | damaging |
| | | | | | | | | 38009 | (0.535) |
| | | | | | | | | 7 | |
| 2150 | 2150 | 5'UTR | G | G | A | c.- | | ENST | |
| 3317 | 3317 | | | | | 558C > T | | 00000 | |
| | | | | | | | | 60232 | |
| | | | | | | | | 6 | |
| 8984 | 8984 | Missense | A | A | T | c. | p. | ENST | benign |
| 5947 | 5947 | Mutation | | | | 628A > T | N210Y | 00000 | (0.012) |
| | | | | | | | | 37045 | |
| | | | | | | | | 6 | |
| 3735 | 3735 | Splice_ | GCCTT | GCCTT | — | c.724- | p. | ENST | |
| 0366 | 0370 | Site | | | | 3_725del | X242_ | 00000 | |
| | | | | | | | splice | 23149 | |
| | | | | | | | | 8 | |
| 2121 | 2121 | Frame_ | GCAAG | GCAAG | | c.540_ | p. | ENST | |
| 6761 | 6765 | ShiftD | | | | 544del | N180Kfs* | 00000 | |
| | | el | | | | | 17 | 38021 | |
| | | | | | | | | 6 | |
| 1340 | 1340 | Missense | G | G | T | c. | p. | ENST | benign |
| 3837 | 3837 | Mutation | | | | 2541G > T | R847S | 00000 | (0.085) |
| 8 | 8 | | | | | | | 35942 | |
| | | | | | | | | 8 | |
| 1044 | 1044 | Frame_ | — | — | GA | c.611_ | p. | ENST | |
| 1444 | 1444 | Shift_ | | | | 612dup | Q205Sfs* | 00000 | |
| 6 | 7 | Ins | | | | | 22 | 30242 | |
| | | | | | | | | 4 | |
| 1131 | 1131 | Intron | T | T | C | c. | | ENST | |
| 4367 | 4367 | | | | | 2603 + | | 00000 | |
| 0 | 0 | | | | | 1072T > C | | 52466 | |
| | | | | | | | | 5 | |
| 1131 | 1131 | Intron | G | G | A | c. | | ENST | |
| 4367 | 4367 | | | | | 2603 + | | 00000 | |
| 3 | 3 | | | | | 1075G > A | | 52466 | |
| | | | | | | | | 5 | |
| 1149 | 1149 | Frame_ | CGCAGCA | CGCAGCA | | c.2697_ | p. | ENST | |
| 4809 | 4810 | ShiftDel | CAAG | CAAG | | 2707del | L900Kfs* | 00000 | |
| 3 | 3 | | | | | | 17 | 35846 | |
| | | | | | | | | 5 | |
| 5754 | 5754 | Missense | A | A | G | c. | p. | ENST | benign |
| 5433 | 5433 | Mutation | | | | 1532A > G | N511S | 00000 | (0.005) |
| | | | | | | | | 31118 | |
| | | | | | | | | 0 | |
| 7557 | 7557 | Missense | A | A | G | c. | p. | ENST | benign(0) |
| 9353 | 9353 | Mutation | | | | 1204T > C | S402P | 00000 | |
| | | | | | | | | 32268 | |
| | | | | | | | | 0 | |
| 7557 | 7557 | Missense | G | G | A | c. | p. | ENST | benign(0) |
| 9373 | 9373 | Mutation | | | | 1184C > T | S395L | 00000 | |
| | | | | | | | | 32268 | |
| | | | | | | | | 0 | |
| 1221 | 1221 | Intron | T | T | C | c. | | ENST | |
| 7661 | 7661 | | | | | 432 + | | 00000 | |
| 3 | 3 | | | | | 3458A > G | | 34433 | |
| | | | | | | | | 7 | |
| 1545 | 1545 | Missense | G | G | A | c. | p | ENST | probably |
| 7094 | 7094 | Mutation | | | | 1718C > T | .A573V | 00000 | damagin |
| 5 | 5 | | | | | | | 36847 | 8(0.985) |
| | | | | | | | | 4 | |
| 1035 | 1035 | Missense | C | C | T | c. | p. | ENST | benign(0) |
| 4319 | 4319 | Mutation | | | | 260G > A | R87K | 00000 | |
| | | | | | | | | 35069 | |
| | | | | | | | | 7 | |
| 1336 | 1336 | Missense | G | G | A | c.4595 | p.Sl | ENST | benign |
| 7344 | 7344 | Mutation | | | | C>T | 532F | 00000 | (0.065) |
| | | | | | | | | 25450 | |
| | | | | | | | | 8 | |
| 1341 | 1341 | Splice_ | T | T | C | c.1046- | p. | ENST | |
| 9064 | 9064 | Site | | | | 2A > G | X349_ | 00000 | |
| | | | | | | | splice | 25450 | |
| | | | | | | | | 8 | |
| 2969 | 2969 | Missense | GG | GG | AA | c.726_ | p. | ENST | |
| 2923 | 2924 | Mutation | | | | 727del | E243K | 00000 | |
| | | | | | | insAA | | 25995 | |
| | | | | | | | | 1 | |
| 6477 | 6477 | Missense | G | G | A | c. | p. | ENST | benign |
| 826 | 826 | Mutation | | | | 1130C > T | P377L | 00000 | (0.005) |
| | | | | | | | | 52507 | |
| | | | | | | | | 4 | |
| 8642 | 8642 | Missense | GG | GG | AA | c.3146_ | p. | ENST | benign |
| 077 | 078 | Mutation | | | | 3147del | P1049L | 00000 | (0.283) |
| | | | | | | insTT | | 40215 | |
| | | | | | | | | 7 | |
| 1659 | 1659 | Missense | G | G | T | c. | p. | ENST | benign |
| 3520 | 3520 | Mutation | | | | 755C > A | P252Q | 00000 | (0.001) |
| | | | | | | | | 26988 | |
| | | | | | | | | 1 | |
| 2985 | 2985 | RNA | AGG | AGG | — | n.620_ | | ENST | |
| 6334 | 6336 | | | | | 622del | | 00000 | |
| | | | | | | | | 38332 | |
| | | | | | | | | 6 | |
| 7481 | 7481 | Intron | C | C | T | c. 906 + | | ENST | |
| 061 | 061 | | | | | 37C > T | | 00000 | |
| | | | | | | | | 29383 | |
| | | | | | | | | 1 | |
| 1200 | 1200 | Frame_ | GCCGATGC | GCCGATGC | — | c.513_ | p. | ENST | |
| 0820 | 0822 | ShiftDel | AGGAGTC | AGGAGTC | | 534del | L172Tfs* | 00000 | |
| 6 | 7 | | GCACAGC | GCACAGC | | | 80 | 34184 | |
| | | | | | | | | 6 | |
| 6844 | 6844 | Missense | A | A | C | C. | p. | ENST | benign |
| 5929 | 5929 | Mutation | | | | 830A > C | N277T | 00000 | (0.005) |
| | | | | | | | | 24963 | |
| | | | | | | | | 6 | |

TABLE 11c
List of HLA 1 and INF Genes Searched

| | Interferon | Interferon | Interferon | Interferon | Interferon | Interferon |
|---|---|---|---|---|---|---|
| HLA class | Pathway | Pathway | Pathway | Pathway | Pathway | Pathway |
| I genes | (Reactome) | (Reactome) | (Reactome) | (Reactome) | (Reactome) | (Reactome) |
| HLA-A | AAAS | FLNB | ICAM1 | IFNG | MX1 | OAS2 |
| HLA-B | ABCE1 | GBP1 | IFI27 | IFNGR1 | MX2 | OAS3 |
| HLA-C | ADAR | GBP2 | IFI30 | IFNGR2 | NCAM1 | OASL |
| B2M | ARIH1 | GBP3 | IFI35 | IP6K2 | NDC1 | PDE12 |
| TAP1 | B2M | GBP4 | IFI6 | IRF1 | NEDD4 | PIAS1 |
| TAP2 | BST2 | GBP5 | IFIT1 | IRF2 | NUP107 | PIN1 |
| TAPBP | CAMK2A | GBP6 | IFIT2 | IRF3 | NUP133 | PLCG1 |
| PSMB8 | CAMK2B | GBP7 | IFIT3 | IRF4 | NUP153 | PML |
| PSMB9 | CAMK2D | HERC5 | IFITM1 | IRF5 | NUP155 | POM121 |
| NLRC5 | CAMK2G | HLA-A | IFITM2 | IRF6 | NUP160 | POM121C |
| TAPBPL | CD44 | HLA-B | IFITM3 | IRF7 | NUP188 | PPM1B |
| ERAP1 | CIITA | HLA-C | IFNA1 | IRF8 | NUP205 | PRKCD |
| ERAP2 | DDX58 | HLA-DPA1 | IFNA10 | IRF9 | NUP210 | PSMB8 |
| CIITA | EGR1 | HLA-DPB1 | IFNA13 | ISG15 | NUP214 | PTAFR |
| CALR | EIF2AK2 | HLA-DQA1 | IFNA14 | ISG20 | NUP35 | PTPN1 |
| CANX | EIF4A1 | HLA-DQA2 | IFNA16 | JAKI | NUP37 | PTPN11 |
| PDIA3 | EIF4A2 | HLA-DQB1 | IFNA17 | JAK2 | NUP42 | PTPN2 |
| CREB1 | EIF4A3 | HLA-DQB2 | IFNA2 | KPNA1 | NUP43 | PTPN6 |
| HLA-E | EIF4E | HLA-DRA | IFNA21 | KPNA2 | NUP50 | RAEI |
| HLA-F | EIF4E2 | HLA-DRB1 | IFNA4 | KPNA3 | NUP54 | RANBP2 |
| HLA-G | EIF4E3 | HLA-DRB3 | IFNA5 | KPNA4 | NUP58 | RNASEL |
| NFYA | EIF4G1 | HLA-DRB4 | IFNA6 | KPNA5 | NUP62 | RPS27A |
| NFYB | EIF4G2 | HLA-DRB5 | IFNA7 | KPNA7 | NUP85 | RSAD2 |
| NFYC | EIF4G3 | HLA-E | IFNA8 | KPNB1 | NUP88 | SAMHD1 |
| RFX5 | FCGR1A | HLA-F | IFNAR1 | MAPK3 | NUP93 | SEC13 |
| RFXANK | FCGR1B | HLA-G | IFNAR2 | MID1 | NUP98 | SEH1L |
| RFXAP | FLNA | HLA-H | IFNB1 | MT2A | OAS1 | SOCS1 |

| Interferon | Interferon |
|---|---|
| Pathway | Pathway |
| (Reactome) | (Reactome) |
| SOCS3 | TRIM8 |
| SP100 | TYK2 |
| STAT1 | UBA52 |
| STAT2 | UBA7 |
| SUMO1 | UBB |
| TPR | UBC |
| TRIM10 | UBE2E1 |
| TRIM14 | UBE2L6 |
| TRIM17 | UBE2N |
| TRIM2 | USP18 |
| TRIM21 | USP41 |
| TRIM22 | VCAM1 |
| TRIM25 | XAF1 |
| TRIM26 | |
| TRIM29 | |
| TRIM3 | |
| TRIM31 | |
| TRIM34 | |
| TRIM35 | |
| TRIM38 | |
| TRIM45 | |
| TRIM46 | |
| TRIM48 | |
| TRIM5 | |
| TRIM6 | |
| TRIM62 | |
| TRIM68 | |

TABLE 11d
Promoter Methylation Data

| | | MCC- | MCC− | MCC− | MCC− | MCC- | MCC- | | |
|---|---|---|---|---|---|---|---|---|---|
| Sample | | MCC- | MCC- | MCC- | MCC- | 336 | 350 | MCC- | MCC- |
| Viral Status | | 282 | 290 | 301 | 320 | pos | neg | 367 | 2314 |
| IFN | | neg | neg | pos | neg | non- | non- | pos | pos |
| Responsive- | | IFN- | IFN- | IFN- | IFN- | IFN- | IFN- | IFN- | IFN- |
| ness | Gene | responsive | responsive | responsive | responsive | responsive | responsive | responsive | responsive |
| | HLA- | 0.153 | 0.362 | 0.149 | 0.579 | 0.276 | 0.141 | 0.236 | 0.129 |
| | A | | | | | | | | |
| | HLA- | 0.19 | 0.172 | 0.149 | 0.202 | 0.149 | 0.135 | 0.116 | 0.24 |
| | B | | | | | | | | |
| | HLA- | 0.192 | 0.199 | 0.188 | 0.234 | 0.113 | 0.168 | 0.127 | 0.191 |
| | C | | | | | | | | |
| | B2M | 0.221 | 0.325 | 0.188 | 0.4 | 0.257 | 0.301 | 0.151 | 0.21 |
| | TAP1 | 0.467 | 0.44 | 0.497 | 0.491 | 0.491 | 0.416 | 0.404 | 0.525 |
| | TAP2 | 0.577 | 0.667 | 0.689 | 0.676 | 0.65 | 0.553 | 0.597 | 0.655 |
| | TAPBP | 0.268 | 0.517 | 0.267 | 0.534 | 0.56 | 0.429 | 0.135 | 0.39 |
| | PSMB8 | 0.313 | 0.359 | 0.346 | 0.351 | 0.394 | 0.339 | 0.253 | 0.411 |
| | PSMB9 | 0.167 | 0.202 | 0.175 | 0.191 | 0.224 | 0.172 | 0.126 | 0.23 |
| | NLRC5 | 0.785 | 0.776 | 0.839 | 0.824 | 0.785 | 0.651 | 0.727 | 0.842 |

10 of 11 MCC lines exhibited strikingly exhibited low, nearly absent, surface HLA-I by flow cytometry (FIG. 1D). This low surface HLA-I was similar to well-studied MCPyV+ lines MKL-1 and WaGa (FIG. 1L). Three lines (MCC-336, -350, -358) did not appreciably upregulate HLA-I after IFN-γ exposure (≤1.15-fold increase in MFI), whereas 8 lines exhibited a ≥2.5 fold increase (median 5.7, range 2.5-12.4). It was further confirmed in two lines that IFN-α-2b and IFN-β upregulate HLA-I (FIG. 1M), while IFN-γ also upregulated HLA-DR expression in the MCC-301 cell line (FIG. 1N).

These cell line results were consistent with the immunohistochemistry (IHC) characterization of HLA-I expression on 9 corresponding parental tumors, in which the majority (6 of 9) displayed HLA-I-positive staining in less than 15% of tumor cells (FIG. 1J; FIG. 2I), as well as minimal HLA class II (FIG. 1R). The tumor-infiltrating CD8⁺ T cell density (median 56.6 cells/mm², range 0-1031.8) was on par with previous reports for MCC (FIG. 1S). Moreover, the availability of serial formalin-fixed paraffin-embedded (FFPE) tumor samples allowed us to then assess changes in HLA-I expression over time. All cell lines except MCC-290 were derived from post-treatment tumors, most commonly radiation±cisplatin/etoposide (Table 6), and pre-treatment samples were available for 6 patients. In 5 of 6 cases, the post-treatment specimen demonstrated fewer HLA-I-positive cells than the paired pre-treatment specimens (FIG. 1O), further implicating HLA-I loss as a mechanism of therapeutic resistance.

### Example 12: MCC Lines Exhibit Transcriptional Downregulation of Multiple Class I Genes and NLRC5 Alterations

To elucidate the mechanisms of impaired HLA-I surface expression in our MCC lines, n in-depth genomic and transcriptional characterization for a subset of MCPyV+ and MCPyV− lines for which material was available was performed (Table 11). To define class I APM transcriptional alterations, the transcriptomes of all 11 MCC lines before and after IFN-γ stimulation was evaluated. At baseline, the MCC lines exhibited low expression of HLA-B, TAP1, TAP2, PSMB8, and PSMB9, compared to control epidermal keratinocytes and dermal fibroblasts, which are candidates for the cell-of-origin of MCPyV− and MCPyV+ MCC, respectively (FIG. 2A). IFN-γ treatment markedly upregulated class I gene transcripts (FIG. 2B; Table 11), a trend which was confirmed in matched proteomes in 4 MCC lines (FIG. 2C). Non-IFN-γ-responsive lines (FIG. 1J) exhibited variable defects, such as lack of IFN-induced HLA-A, -B, and -C mRNA upregulation in MCC-336 (FIG. 2A) and global lack of IFN-induced HLA-I and IFN pathway upregulation at the protein level in MCC-350, including lack of STAT1 phosphorylation (FIG. 2C; FIG. 2D-E).

To investigate the heterogeneity in the HLA-I downregulation observed in the bulk RNA-seq data, high-throughput, droplet-based single-cell transcriptome sequencing of 2 fresh MCC biopsies (MCC-350 [MCPyV−] and MCC-336 [MCPyV+]) was performed. From a total of 15,808 cells (mean 4,231.9 genes/cells) identified across the two samples, 7 distinct transcriptionally defined clusters were detected. CD45+ immune cells comprised cluster 6, while clusters 0-5 were MCC cells, identified by the expression of SOX2, SYP, and ATOH1 (FIG. 2F; FIG. 2G). All MCC clusters displayed nearly absent HLA-B, TAP1/2, PSMB8/9, and NLRC5 expression and low HLA-A and -C expression (FIG. 2F; FIG. 2H), consistent with the bulk RNA-seq data. By contrast, cluster 6 (immune cells) displayed an average 21-fold higher levels of HLA-A, -B, and -C transcripts.

Given the marked RNA- and protein-level downregulation of class I genes at baseline, possible genetic basis for these observations was investigated. By WES, no MCC lines harbored any notable mutations in class I APM genes, except for HLA-F and -H mutations in MCC-320 (Table 11). While a total of 32 mutations were detected in IFN pathway genes across all analyzed lines, only 2 were predicted as probably damaging by Polyphen and no mutations were detected in IFNGR1/2, JAK1/2, STAT1, or IRF1/2 (Table 11). However, copy number loss of NLRC5 was detected in 5 of 8 lines (62.5%) analyzed (FIG. 2I; Table 11). NLRC5 is a transcriptional activator of several class I pathway genes that localize to conserved S/X/Y regions in their promoters. NLRC5 copy number loss has been recently recognized as a common alteration across many cancers.

### Example 13: IFN-γ-Induced HLA-I Upregulation is Associated with Shifts in the HLA Peptidome

Diminished expression of HLA-I would be expected to result in a lower number and diversity of HLA-presented peptides in MCC, impacting the immunogenicity of the tumor. Indeed, using workflows for direct detection of class I-bound peptides by liquid chromatography tandem mass spectrometry (LC-MS/MS) (Methods), after immunoprecipitation of tumor cell lysates with a pan-HLA-I antibody (FIG. 3M), similarly low total peptide counts at baseline in parental tumors and cell lines were detected (FIG. 3A-B). Following IFN-γ stimulation, a median 12-fold increase in the abundance of class I bound peptides was detected across 7 cell lines using comparable input material for immunoprecipitation (FIG. 3K, FIG. 3A-B, Methods). The baseline immunopeptidome amino acid signature between the cell lines and parental tumors were highly correlated (FIG. 3C), and the cell line peptidomes shared more than 50% of their peptides with the corresponding tumor peptidomes (FIG. 3D). In contrast, lower correlations before and after IFN-γ treatment and altered overall binding motifs with IFN-γ exposure was observed (FIGS. 3K and 3E, FIG. 3N). To further explore these observations, the most likely HLA allele bound by the identified peptides was inferred. When comparing cell lines with and without IFN-γ treatment, dramatic changes in the frequencies of peptides mapping to each HLA allele was observed, most notably an increase in HLA-B-presented peptides (FIG. 3F-G).

For the MCPyV+ lines, it was hypothesized that this upregulation of HLA-I following IFN-γ stimulation would lead to increased ability to present MCPyV-specific epitopes. Indeed, for the MCPyV+ line MCC-367, a peptide sequence derived from the origin-binding domain (OBD) of LT antigen (TSDKAIELY) was detected, which was predicted as a strong binder for the HLA*A01:01 allele of that cell line (rank=0.018, HLAthena) (FIG. 3J, Methods). Reactivity against this MCC-367 derived epitope was confirmed by autologous T cells by ELISpot assay, demonstrating the immunogenicity of this epitope (FIG. 3L).

### Example 14: Complementary Genome-Scale Gain- and Loss-of-Function Screens to Identify Novel Regulators of HLA-I in MCC

The simultaneous transcriptional downregulation of multiple class I APM genes suggested that this suppression was coordinated by upstream regulators. While NLRC5 copy number loss was a notable event, it was only observed in 5 of 8 lines (62.5%) studied, and thus the presence of other regulators was suspected. To this end, a paired genome-scale open reading frame (ORF) gain-of-function and CRISPR-Cas9 knock out (KO) loss-of-function screens in the MCPyV+ MCC-301 line was generated to systematically identify novel regulators of HLA-I surface expression in MCC. MCC-301 line was chosen for three reasons. First, the low TMB of MCPyV+ MCC increases the likelihood of a homogeneous mechanism of HLA-I suppression, which might relate to viral antigen signaling or cell-type specific factors. Second, IFN-γ-mediated inducibility of HLA-I largely excludes the possibility of hard-wired genomic alterations that would prohibit HLA-I upregulation. Last, such screens necessitate cell lines with robust growth such as MCC-301 (FIG. 1P).

MCC-301 cells were transduced at a low multiplicity of infection with genome-scale ORF or Cas9+sgRNA lentiviral libraries (Methods). After staining cells with an anti-HLA-ABC antibody, HLA-I-high and HLA-I-low populations underwent fluorescence activated cell sorting (FACS)-based cell isolation, with each screen performed in triplicate (FIG. 4A). Constructs were ranked according to their median log 2-fold change (LFC) enrichment in the HLA-I-high versus HLA-I-low populations and for the CRISPR screen, sgRNA rankings were aggregated into gene-level rankings using the STARS algorithm (Methods).

### Example 15: MYCL Identified as a Mediator of HLA-I Suppression in MCC Via ORF Screen

The ORF screen produced 75 hits with a >4-fold enrichment in HLA-I-high versus HLA-I-low populations. As expected, these hits were highly enriched for IFN and HLA-I pathway genes by Gene Set Enrichment Analysis (GSEA) (FIG. 4D,). The top hit was IFNG, with IFN pathway genes comprising 4 of the top 12 hits (33%). HLA-B and -C were ranked #10 and #38. Of note, transduction with the ORF library led to a population-wide increase in HLA-I, presumably due to IFN secretion from cells transduced with IFN gene ORFs. An ORF library-specific effect was confirmed and not due to lentiviral transduction, as GFP-transduced cells did not exhibit an increase in surface HLA-I (FIG. 4C). Furthermore, it was confirmed that these notable hits exhibited high concordance between at least 2 replicates (FIG. 4F-I).

The many highly enriched positive hits were validated by generating 71 single ORF overexpression lines in MCC-301, focusing on the top positive hits not directly related to IFN or HLA-I pathways. By flow cytometry, 8 of 71 candidate hits (11.3%) upregulated surface HLA-I by >2-fold compared to a GFP control while also maintaining viability after transduction, including Polycomb-related genes EZHIP (CXorf67) and YY1 (FIG. 411). As further validation, ORFs were transduced into the MCPyV+ MCC-277 line and confirmed increased levels of HLA-I (FIG. 411). In contrast to the genes that increased levels of HLA-I, MYCL was the top negative hit (FIG. 4D). MYCL is an important transcription factor in MCPyV+ MCC, as ST binds and recruits MYCL to the EP400 chromatin modifier complex to enact widespread epigenetic changes necessary for oncogenesis. As validation, it was observed that MYCL knockdown in MKL-1 cells resulted in an increase in surface HLA-I by flow cytometry compared to a scrambled shRNA control (P=0.003), an effect which was negated by rescue expression of exogenous MCYL (FIG. 4M).

To further investigate how MYCL affects HLA-I surface expression, RNA-seq of the MKL-1 MYCL shRNA line was performed. Compared to the scrambled shRNA control line, a >2-fold increase in expression of class I genes including HLA-B, HLA-C, TAP1, and PSMB9, was observed, with enrichment for the signature of antigen processing/presentation by GSEA (q=0.04; FIG. 4N, FIG. 5B,). Since ST binds and potentiates MYCL function through the ST-EP400-MYCL complex, it was suspected that viral antigen inactivation might also upregulate class I. To further expand the scope of these findings, another established MCPyV+ MCC line, WaGa, was selected to transduce with an shRNA that targets shared exons of ST and LT, leading to inactivation of both MCPyV viral antigens. A similar but more modest upregulation of class I genes, including >1.5-fold increases in HLA-B, HLA-C, and NLRC5, was observed (FIG. 4O;). Moreover, knockdown of EP400 in MKL-1 with two different shRNAs resulted in >3-fold increases in HLA-B and HLA-C (FIG. 5J). These findings thus implicate the continued expression of ST-EP400-MYCL complex components in the downregulation of HLA-I in MCC.

To determine if the HLA-I-suppressive effects of MYCL generalized to MCPyV− MCC and other cancers, the copy number status of MYCL in MCPyV− MCC was evaluated. Copy number gain of chromosome 1p, encompassing MYCL, was previously reported as one of the more common copy number alterations in MCC. Three of the 4 (75%) MCPyV− MCC lines exhibited MYCL copy number gain (copy number ratio 1.16-1.56; FIG. 5E), suggesting a mechanism by which MCPyV− MCC may enhance MYCL signaling in the absence of viral antigens. To determine if MYCL is related to HLA-I expression in other cancers, RNA-seq data from the Cancer Cell Line Encyclopedia was searched. Notably, other neuroendocrine cancers such as small cell lung carcinoma and neuroblastoma with lower expression of HLA-I pathway components also frequently featured overexpression of MYC family members MYCL and MYCN, respectively (FIG. 5F). Overall, MYCL exhibited negative correlation with average HLA-I gene expression (Pearson correlation r=−0.33, P=0.04).

### Example 16: PRC1.1 Complex Identified as a Novel Negative Regulator of HLA-I in MCC by CRISPR Loss-of-Function Screen

The CRISPR-KO screen also identified several class I APM genes. The top negative hit was TAPBP (FIG. 4E,), a chaperone for partially folded HLA-I heavy chains that facilitates binding between unbound HLA-I and TAP. Other notable negative hits included IFN pathway gene IRF1 (#21) and class I genes CALR (#84) and B2M (#141). Having previously identified MYCL in the ORF screen, it was observed other ST-MYCL-EP400 complex members within the CRISPR positive hits included BRD8 (#51), DMAP1 (#93), KAT5 (#619), and EP400 (#886). In addition, several components of the Polycomb repressive complex 1.1 (PRC1.1) within the CRISPR positive hits were identified, including the top two hits of the screen: USP7 (#1), BCORL1 (#2), and PCGF1 (#50). For these genes, high concordance between two CRISPR replicates was observed (FIGS. 4F and I; Methods) and a >4.5-fold enrichment for at least 2 of the 4 sgRNAs (FIG. 4G). PRC1.1 is a noncanonical Polycomb repressive complex that silences gene expression through mono-ubiquitination of H2AK119 in CpG islands. Other components of PRC1.1 include KDM2B, SKP1, RING1A/B, RYBP/YAF2, and BCOR (which can substitute for BCORL1). In aggregate, review of the top hits across the parallel screens revealed several hits related to Polycomb repressive complexes: PRC1.1 components USP7, BCORL1, and PCGF1; ORF hits EZHIP, which is an inhibitor of Polycomb repressive complex 2 (PRC2), and YY1; and PRC2 components EED and SUZ12 (CRISPR positive hits #162 and #409).

A series of MCC-301 KO lines against PRC1.1 genes USP7, BCORL1, and PCGF1 were generated. Compared to a non-targeting sgRNA control line, knockout of each gene increased baseline surface HLA-I expression levels as assessed by flow cytometry (FIG. 511). PCGF1 knockout increased IFN-γ-induced HLA-I upregulation as well (FIG. 4P). Gene editing and protein knockout were confirmed by Sanger sequencing using TIDE (FIG. 4L) and by western blot (FIG. 511), in genes for which antibodies were available.

To define the specific class I APM gene expression changes associated with PRC1.1 loss of function, RNA-seq data from a PCGF1-KO line and a non-targeting sgRNA control line in MCC-301 was generated, since previous studies demonstrated that PCGF1 is essential for PRC1.1 function. Genes upregulated in the PCGF1-KO line were significantly enriched for the “PRC2 target genes” signature (FIG. 5I), consistent with the known role of PRC1.1 in coordinating with PRC2 to repress target genes. Strikingly, a >5-fold increase in expression of the class I APM genes TAP1, TAP2, and PSMB8, with a more modest increase in the class I transactivator NLRC5 was noticed (FIG. 5I). For further confirmation, increased protein expression of TAP1 by Western blot both at baseline and after IFN-γ treatment in the PCGF1-KO line was observed (FIG. 5J). An RNA-seq cohort of 51 MCC tumor biopsies was evaluated to examine the association between expression of HLA-I genes and PRC1.1. To account for the potential of immune cell infiltration, which might confound measurement of bulk class I expression, ESTIMATE⁵¹ was applied to calculate tumor purity (median 87% purity, range 41-99%). A negative correlation was observed between several class I genes and PRC1.1 components BCOR and KDM2B (P<0.05; FIG. 5G).

To explore if there is a relationship between MYCL and PRC1.1, previously generated ChIP-seq data in MKL-1 cells was analyzed. It was observed that components of the ST-MYCL-EP400 complex were bound to the promoters of PRC1.1 genes USP7 and PCGF1, but not BCOR or BCORL1 (FIG. 6A, FIG. 6B). The binding of MAX and EP400 to USP7 and PCGF1 was further confirmed by ChIP qPCR (FIG. 6G). These results indicate that PRC1.1 may act downstream of MYCL. Moreover, both MYCL and PRC1.1 component USP7 encode proteins that have been reported to directly interact with MCPyV ST and LT viral antigens, respectively, suggesting a model by which viral antigens may coordinate via MYCL and PRC1.1 to suppress HLA-I surface expression (FIG. 4K).

### Example 17: Pharmacologic Inhibition of USP7 Restores HLA-I in MCC

Selective small-molecule inhibitors of the PRC1.1 component USP7 have been previously developed. However, since USP7 has many functions, such as regulation of p53 through MDM2 deubiquitination, and since its association with PRC1.1 was recently discovered, the extent of USP7's role in PRC1.1 was investigated. By examining the Cancer Dependency Map, genes whose survival dependency correlated with that of USP7 across cancer cell lines were identified, with the rationale that survival co-dependency implies that such genes may function within the same complex or pathway. While TP53-wildtype (WT) lines did not exhibit co-dependency between USP7 and Polycomb genes, TP53-mutant lines showed a high correlation between USP7 and PRC1.1 genes PCGF1 and RING1 (6^{th} and 13^{th} highest correlation coefficients, FDR=2.46×10⁻⁴ and 2.97×10⁻³, respectively) (FIG. 6E,). Furthermore, GSEA analysis revealed histone ubiquitination as the most enriched gene set within USP7 co-dependent genes in TP53-mutant cell lines (FIG. 6F,). These results further support the notion that USP7 plays an important role in PRC1.1 function.

The activity of XL177A, a potent and irreversible USP7 inhibitor, was compared to XL177B, the enantiomer of XL177A which is 500-fold less potent but exhibits on-target activity at higher doses. Two MCPyV+ lines (MCC-301 and -277) and two MCPyV− lines (MCC-290 and -320) were treated for 3 days at varying inhibitor concentrations. At 100 nM, a mean 2.0-fold (range 1.78-2.27) increase was observed in expression of surface HLA-I by flow cytometry relative to DMSO in the two MCPyV+ lines. Within the MCPyV− lines, a more modest increase in HLA-I levels in MCC-290 but not MCC-320 was noted (FIG. 6D). Given USP7's prominent role in p53 regulation, it was assessed if USP7's effect on HLA was p53-dependent. Notably, XL177A treatment of both TP53-KO and TP53-WT lines in MKL-1 increased surface HLA-I relative to XL177B and DMSO (FIG. 6H; FIG. 6K). Moreover, while USP7 inhibition did induce slight cell cycle shifts from S to G1 phase, this effect was similar in both TP53-WT and TP53-KO contexts (FIG. 6L). To evaluate the functional consequences of USP7 inhibition on HLA-I presentation, the HLA-I-bound peptidomes of MCC-301 cells treated with XL177A and XL177B was analyzed. XL177A-treated cells exhibited higher abundances of displayed peptides compared to XL177B and untreated cells (FIG. 6I,). Out of 282 peptides whose abundance significantly differed (P<0.05) between two of the three conditions, 270 peptides (95.7%) were more abundant in XL177A compared to untreated cells. Notably, XL177A treatment did not affect the frequency of peptides displayed on each respective HLA-I gene (HLA-A, -B, -C) (FIG. 6J).

### Example 18: Discussion

Surface HLA-I loss is a widespread mechanism of immune evasion in cancer and facilitates resistance to immunotherapy. As a virally driven cancer, MCPyV+ MCC provides a highly informative substrate to study mechanisms by which viral antigens corrupt normal physiology. Applicant suspected that MCPyV viral antigens also suppress class I antigen presentation through derangement of regulatory mechanisms that might be phenocopied in other cancers including MCPyV− MCC tumors. Through unbiased genome-scale screens for regulators of HLA-I, MYCL was identified, which acts as part of the ST-MYCL-EP400 complex in MCPyV+ MCC and is frequently amplified in MCPyV− MCC. The ST antigen recruits MYCL to the EP400 complex to enact widespread epigenetic changes necessary for MCC oncogenesis, and the results herein identify a novel function of ST in suppressing HLA-I by MYCL activity. The effect of MYC family proteins on HLA generalizes to other cancers as well, as MYC and MYCN can suppress HLA-I in melanoma and neuroblastoma, respectively.

The identification of PRC1.1 in the CRISPR screen clearly confirms the importance of epigenetic regulatory mechanisms in suppressing HLA-I. PRC1.1 is a noncanonical Polycomb complex that mono-ubiquitinates H2AK119 within CpG islands, facilitating recruitment of PRC2 which deposits suppressive H3K27 trimethylation marks. PRC2 was recently identified as an HLA-I repressor through independent CRISPR screens in leukemia and lymphoma cell lines, and this work establishes a novel connection to PRC1.1. Those screens also identified PCGF1 ( ), and PRC2 subunits were identified in the CRISPR screen and PRC2 inhibitor EZHIP in the ORF screen.

Reversal of HLA-I loss is crucial for an effective anti-tumor cytotoxic T cell response, and, of high clinical interest, an HLA-I-upregulating drug could augment response to immunotherapy such as checkpoint blockade. The small-molecule USP7 inhibitor studies herein provide an avenue for pharmacologic upregulation of HLA-I in MCC via PRC1.1 inhibition. In contrast to the nonspecific, inflammatory mechanism by which IFN-γ upregulates HLA-I, USP7 inhibition reverses the underlying tumor-intrinsic, epigenetic defects in class I antigen presentation via disruption of PRC1.1. Thus, USP7 inhibition raises baseline tumor HLA-I expression without the requirement of an inflammatory microenvironment.

The USP7 and PCCGF1 promoter occupation by the ST-MYCL-EP400 complex suggests a possible unifying mechanism by which MCPyV ST antigen co-opts MYCL to increase expression of PRC1.1, which subsequently suppresses class I APM gene expression.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Information (NCBI) on the World Wide Web at ncbi.nlm.nih.gov.

### EQUIVALENTS

Those skilled in the art will recognize, or be able to ascertain using no more than routine experimentation, many equivalents to the specific embodiments encompassed by the present invention described herein. Such equivalents are intended to be encompassed by the following claims.

What is claimed is:

1. A method of treating a subject afflicted with a cancer comprising administering to the subject a therapeutically effective amount of an agent that modifies the copy number, the expression level, and/or the activity of one or more biomarkers listed in Table 1, 2, 3, 4, or 5 or a fragment thereof, and an immunotherapy.

2. The method of claim 1, wherein the agent decreases the copy number, the expression level and/or the activity of one or more biomarkers listed in Table 1 or 4 or a fragment thereof.

3. The method of claim 1 or 2, wherein the agent decreases the copy number, the expression level, and/or the activity of MYCL polypeptide and/or a polycomb repressor complex 1.1 (PRC1.1) polypeptide, or polynucleotide encoding the polypeptide.

4. The method of claim 3, wherein the polycomb repressor complex 1.1 (PRC1.1) polypeptide is USP7, BCORL1, PCGF1, KDM2B, SKP1, RING1A, RING1B, RYBP, YAF2, and/or BCOR.

5. The method of any one of claims 1-4, wherein the agent is a small molecule inhibitor, CRISPR single-guide RNA (sgRNA), RNA interfering agent, antisense oligonucleotide, peptide or peptidomimetic inhibitor, aptamer, antibody, or intrabody.

6. The method of claim 5, wherein the RNA interfering agent is a small interfering RNA (siRNA), CRISPR RNA (crRNA), a small hairpin RNA (shRNA), a microRNA (miRNA), or a piwi-interacting RNA (piRNA).

7. The method of claim 6, wherein the sgRNA comprises a nucleic acid sequence selected from the group consisting of nucleic acid sequence listed in Tables 1-4.

8. The method of claim 5, wherein the agent comprises an intrabody, or an antigen binding fragment thereof, that specifically binds to the one or more biomarkers and/or a substrate of the one or more biomarkers listed in Table 1, 2, 3, 4, or 5.

9. The method of claim 8, wherein the intrabody, or antigen binding fragment thereof, is a murine, chimeric, humanized, composite, or human intrabody, or antigen binding fragment thereof.

10. The method of claim 8 or 9, wherein the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabody fragments.

11. The method of claim 1, wherein the agent increases the copy number, the expression level and/or the activity of one or more biomarkers listed in Table 2 or 3 or a fragment thereof.

12. The method of any one of claims 1-10, wherein the agent increases the sensitivity of the cancer cells to an immunotherapy.

13. The method of any one of claims 1-11, wherein the immunotherapy is administered before, after, or concurrently with the agent.

14. The method of claim 12 or 13, wherein the immunotherapy comprises an anti-cancer vaccine and/or virus.

15. The method of any one of claims 12-14, wherein the immunotherapy is a cell-based immunotherapy, optionally wherein the cell-based immunotherapy is chimeric antigen receptor (CAR-T) therapy.

16. The method of any one of claims 12-15, wherein the immunotherapy inhibits an immune checkpoint.

17. The method of claim 16, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR.

18. The method of claim 17, wherein the immune checkpoint is selected from the group consisting of PD-1, PD-L1, and PD-L2, optionally wherein the immune checkpoint is PD-1.

19. The method of any one of claims 1-10, wherein the one or more biomarker comprises a nucleic acid sequence having at least 95% identity to a nucleic acid sequence listed in Table 5 and/or encodes an amino acid sequence having at least 95% identity to an amino acid sequence listed in Table 5.

20. The method of any one of claims 1-19, wherein the subject is a mammal.

21. The method of any one of claims 1-20, wherein the subject is a human, non-human primate, mouse, rat, or domesticated mammal.

22. The method of any one of claims 1-21, wherein the agent increases the sensitivity of the cancer to the immunotherapy, optionally wherein (i) the immunotherapy is T-cell-mediated and/or (ii) the agent increases the amount of CD8+ T cells in a tumor comprising the cancer cells.

23. The method of any one of claims 1-22, wherein the agent increases the level of MHC-I on the surface of the cancer cells.

24. The method of any one of claims 1-23, further comprising administering to the subject at least one additional cancer therapy or regimen.

25. The method of claim 24, wherein the at least one additional cancer therapy or regimen is administered before, after, or concurrently with the agent and/or the immunotherapy.

26. The method of any one of claims 1-25, wherein the agent is administered in a pharmaceutically acceptable formulation.

27. The method of any one of claims 1-26, wherein the cancer is a neuroendocrine cancer.

28. The method of claim 27, wherein the neuroendocrine cancer is a Merkel cell carcinoma, neuroblastoma, small cell lung cancer, or neuroendocrine carcinoma.

29. A method of increasing major histocompatibility complex expression in a cancer cell, the method comprising contacting the cancer cell with an agent that modulates the copy number, the expression level, and/or the activity of one or more biomarkers listed in Table 1, 2, 3, 4, or 5 or a fragment thereof, optionally further comprising contacting the cancer cell, or a population of cells comprising the cancer cell and immune cells, with an immunotherapy.

30. The method of claim 29, wherein the agent that decreases the copy number, the expression level, and/or the activity of one or more biomarkers listed in Table 1 or 4.

31. The method of claim 29, wherein the agent decreases the copy number, the expression level, and/or the activity of MYCL polypeptide and/or a polycomb repressor complex 1.1 (PRC1.1) polypeptide, or polynucleotide encoding the polypeptide.

32. The method of claim 30, wherein the polycomb repressor complex 1.1 (PRC1.1) polypeptide is USP7, BCORL1, PCGF1, KDM2B, SKP1, RING1A, RING1B, RYBP, YAF2, and/or BCOR.

33. The method of any one of claims 29-31, wherein the agent is a small molecule inhibitor, CRISPR single-guide RNA (sgRNA), RNA interfering agent, antisense oligonucleotide, peptide or peptidomimetic inhibitor, aptamer, antibody, or intrabody.

34. The method of claim 32, wherein the RNA interfering agent is a small interfering RNA (siRNA), CRISPR RNA (crRNA), a small hairpin RNA (shRNA), a microRNA (miRNA), or a piwi-interacting RNA (piRNA).

35. The method of claim 33, wherein the sgRNA comprises a nucleic acid sequence selected from the group consisting of nucleic acid sequence listed in Tables 1-4.

36. The method of claim 34, wherein the agent comprises an intrabody, or an antigen binding fragment thereof, that specifically binds to the one or more biomarkers and/or a substrate of the one or more biomarkers listed in Table 1, 2, 3, 4, or 5.

37. The method of claim 32, wherein the intrabody, or antigen binding fragment thereof, is a murine, chimeric, humanized, composite, or human intrabody.

38. The method of claim 36, wherein the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabodies fragments.

39. The method of claim 29, wherein the agent increases the copy number, the expression level, and/or the activity of one or more biomarkers listed in Table 2 or 3.

40. The method of any one of claims 29-38, wherein the agent increases the sensitivity of the cancer cells to the immunotherapy.

41. The method of any one of claims 29-39, wherein the cancer cells are contacted with the immunotherapy before, after, or concurrently with the agent.

42. The method of claim 39 or 40, wherein the immunotherapy comprises an anti-cancer vaccine and/or virus.

43. The method of any one of claims 39-41, wherein the immunotherapy is a cell-based immunotherapy, optionally wherein the cell-based immunotherapy is chimeric antigen receptor (CAR-T) therapy.

44. The method of any one of claims 39-42, wherein the immunotherapy inhibits an immune checkpoint.

45. The method of claim 43, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR.

46. The method of claim 44, wherein the immune checkpoint is selected from the group consisting of PD-1, PD-L1, and PD-L2.

47. The method of claim 45, wherein the immune checkpoint is PD-1.

48. The method of any one of claims 29-37, wherein the biomarker comprises a nucleic acid sequence having at least 95% identity to a nucleic acid sequence listed in Table 5 and/or encodes an amino acid sequence having at least 95% identity to an amino acid sequence listed in Table 5.

49. The method of any one of claims 29-47, wherein the one or more biomarker is a human, mouse, chimeric, or a fusion biomarker.

50. The method of any one of claims 29-48, wherein the immunotherapy is (i) T-cell-mediated and/or (ii) the agent increases the amount of CD8+ T cells in a tumor comprising the cancer cells.

51. The method of any one of claims 29-50, wherein the agent increases the level of MHC class I surface expression in the cancer cells.

52. The method of any one of claims 29-51 further comprising administering to the subject at least one additional cancer therapy or regimen.

53. The method of claim 52, wherein the at least one additional cancer therapy or regimen is administered before, after, or concurrently with the agent and/or the immunotherapy.

54. The method of any one of claims 29-53, wherein the agent is administered in a pharmaceutically acceptable formulation.

55. The method of any one of claims 29-54, wherein the cancer cell is a neuroendocrine cancer cell.

56. The method of claim 55, wherein the neuroendocrine cancer cell is a Merkel cell carcinoma, neuroblastoma, small cell lung cancer, or neuroendocrine carcinoma cell.

57. A method of identifying a subject afflicted with, or at risk for developing, a cancer that can be treated by modulating the copy number, amount, and/or activity of at least one biomarker listed in Table 1, 2, 3, 4, or 5, the method comprising detecting an increased or decreased level of major histocompatibility complex (MHC) class I expression in a cell from the subject relative to a control, thereby identifying the subject afflicted with, or at risk of developing, a cancer that can be treated by modulating the copy number, amount, and/or activity of at least one biomarker listed in Table 1, 2, 3, 4, or 5, optionally wherein a biological sample comprising the cell from the subject is obtained from the subject.

58. The method of claim 57, wherein the agent decreases the copy number, amount, and/or activity of at least one biomarker listed in Table 1 or 4.

59. The method of claim 57 or 58 further comprising recommending, prescribing, or administering to the identified subject an agent that inhibits the at least one biomarker listed in Table 1 or 4.

60. The method of claim 57, wherein the agent increases the copy number, amount, and/or activity of at least one biomarker listed in Table 2 or 3.

61. The method of any one of claims 57-60 further comprising recommending, prescribing, or administering to the identified subject an immunotherapy.

62. The method of claim 61, wherein the immunotherapy comprises an anti-cancer vaccine, an anti-cancer virus, and/or a checkpoint inhibitor.

63. The method of any one of claims 57-61 further comprising recommending, prescribing, or administering to the subject a cancer therapy selected from the group consisting of targeted therapy, chemotherapy, radiation therapy, and/or hormonal therapy.

64. The method of any one of claims 57-63, wherein the control comprises a sample derived from a cancerous or non-cancerous sample from either the patient or a member of the same species to which the patient belongs.

65. The method of claim 64, wherein the control is a known reference value.

66. The method of any one of claims 57-65, wherein the cancer is a neuroendocrine cancer.

67. The method of claim 66, wherein the neuroendocrine cancer is a Merkel cell carcinoma, neuroblastoma, small cell lung cancer, or neuroendocrine carcinoma.

68. A method for predicting the clinical outcome of a subject afflicted with a cancer expressing one or more biomarkers listed in Table 1, 2, 3, 4, or 5 or a fragment thereof to treatment with an immunotherapy, the method comprising:
a) determining the copy number, amount, and/or activity of at least one biomarker listed in Table 1, 2, 3, 4, or 5 in a subject sample;
b) determining the copy number, amount, and/or activity of the at least one biomarker in a control having a good clinical outcome; and
c) comparing the copy number, amount, and/or activity of the at least one biomarker in the subject sample and in the control;
wherein the presence of, or an insignificant change in the copy number, amount, and/or activity of, the at least one biomarker listed in Table 1, 2, 3, 4, or 5 in the subject sample as compared to the copy number, amount and/or activity in the control, is an indication that the subject has a poor clinical outcome.

69. A method for monitoring the treatment of a subject having or suspected of having cancer with an agent that decreases the copy number and/or amount and/or inhibits the activity of at least one biomarker listed in Table 1 or 4 and an immunotherapy, the method comprising: detecting a change or no change in the level of MHC class I expression in a sample derived from the subject at a first time point and the level of MHC class I expression in a sample derived from the subject at a subsequent time point, thereby monitoring the treatment of the subject.

70. A method for monitoring the treatment of a subject having or suspected of having cancer with an agent that increases the copy number and/or amount and/or inhibits the activity of at least one biomarker listed in Table 2 or 3 and an immunotherapy, the method comprising: detecting a change or no change in the level of MHC class I expression in a sample derived from the subject at a first time point and the level of MHC class I expression in a sample derived from the subject at a subsequent time point, thereby monitoring the treatment of the subject.

71. A method of assessing the efficacy of an agent that decreases the copy number, amount, and/or the activity of at least one biomarker listed in Table 1 or 4 in a subject, the method comprising detecting in a subject sample at a first time point a change or no change in the copy number, amount, and/or activity of at least one biomarker listed in Table 1 or 4 relative to a subsequent time point, wherein a decrease in the copy number, amount, and or activity of at least one biomarker listed in Table 1 or 4 indicates the agent is effective.

72. A method of assessing the efficacy of an agent that increases the copy number, amount, and/or the activity of at least one biomarker listed in Table 2 or 3 in a subject, the method comprising detecting in a subject sample at a first time point a change or no change in the copy number, amount, and/or activity of at least one biomarker listed in Table 2 or 3 relative to a subsequent time point, wherein a decrease in the copy number, amount, and or activity of at least one biomarker listed in Table 2 or 3 indicates the agent is effective.

73. The method of claim 71 or 72, wherein between the first point in time and the subsequent point in time, the subject has undergone treatment, completed treatment, and/or is in remission for the cancer.

74. The method of claim 73, wherein treatment comprises administering the agent to the subject.

75. The method of any one of claims 71-74, wherein the first and/or the subsequent sample comprises ex vivo or in vivo samples.

76. The method of any one of claims 71-75, wherein the first and/or at least one subsequent sample is a portion of a single sample or pooled samples obtained from the subject.

77. The method of any one of claims 71-76, wherein the sample comprises cells, serum, peritumoral tissue, and/or intratumoral tissue obtained from the subject.

78. The method of any one of claims 71-77, wherein the one or more biomarkers listed in Table 1, 2, 3, 4, or 5.

79. The method of any one of claims 1-78, wherein the cancer or cancer cell is a neuroendocrine cancer.

80. The method of claim 79, wherein the neuroendocrine cancer is a Merkel cell carcinoma, neuroblastoma, small cell lung cancer, or neuroendocrine carcinoma.

81. The method of any one of claims 1-80, wherein the cancer or cancer cell is in an animal model of the cancer.

82. The method of claim 81, wherein the animal model is a mouse model.

83. The method of any one of claims 1-82, wherein the cancer is in a mammalian subject.

84. The method of claim 83, wherein the mammalian subject is a mouse or a human.

85. The method of claim 84, wherein the mammal is a human.


SEQUENCE LISTING (88 sequences)

SEQ ID NO: 1 - DNA, 5831 bp, Homo sapiens
gacatttcacgccgccgccattttgagagcgagccgagccgagctgccgggcgccgcgtccgctgccggagccccgacgacgacgccgaggaggcggaggccgcggctctcggaacgcggccggcccgtcgcccgcccgctccgccgctcccgccggccccagggcccgcaggcccgccgcggccggccaggcctcccgtccgccgcgcccggcccgaggcggggctgactccgcggcccccgaccggccgccctccgcccccggccggcccgcggccccgcagccccggccccggccccggcggcgggaggcgggccccgcggcggcggcggcggcggccgcagcgagcgacgaggccgcccggcccggcccgccggccgcccgcccgcctcggcgccgagattgggcgaatcgcgagcaagtacgtgcgcgtctccctgccgccgccgccgcccgccgcgggccgccccggggccgccgtcgccgacgacgcgcgggaggaggaggaggaggccgccccgccgccgccgccgccgccgccgccccggctcgccgccgcccgcccgccgggctcgcagccccggcccccggccgcaggcgaggcccaggccgcggccgacatgaaccaccagcagcagcagcagcagcagaaagcgggcgagcagcagttgagcgagcccgaggacatggagatggaagcgggagatacagatgacccaccaagaattactcagaaccctgtgatcaatgggaatgtggccctgagtgatggacacaacaccgcggaggaggacatggaggatgacaccagttggcgctccgaggcaacctttcagttcactgtggagcgcttcagcagactgagtgagtcggtccttagccctccgtgttttgtgcgaaatctgccatggaagattatggtgatgccacgcttttatccagacagaccacaccaaaaaagcgtaggattctttctccagtgcaatgctgaatctgattccacgtcatggtcttgccatgcacaagcagtgctgaagataataaattacagagatgatgaaaagtcgttcagtcgtcgtattagtcatttgttcttccataaagaaaatgattggggattttccaattttatggcctggagtgaagtgaccgatcctgagaaaggatttatagatgatgacaaagttacctttgaagtctttgtacaggcggatgctccccatggagttgcgtgggattcaaagaagcacacaggctacgtcggcttaaagaatcagggagcgacttgttacatgaacagcctgctacagacgttatttttcacgaatcagctacgaaaggctgtgtacatgatgccaaccgagggggatgattcgtctaaaagcgtccctttagcattacaaagagtgttctatgaattacagcatagtgataaacctgtaggaacaaaaaagttaacaaagtcatttgggtgggaaactttagatagcttcatgcaacatgatgttcaggagctttgtcgagtgttgctcgataatgtggaaaataagatgaaaggcacctgtgtagagggcaccatacccaaattattccgcggcaaaatggtgtcctatatccagtgtaaagaagtagactatcggtctgatagaagagaagattattatgatatccagctaagtatcaaaggaaagaaaaatatatttgaatcatttgtggattatgtggcagtagaacagctcgatggggacaataaatacgacgctggggaacatggcttacaggaagcagagaaaggtgtgaaattcctaacattgccaccagtgttacatctacaactgatgagatttatgtatgaccctcagacggaccaaaatatcaagatcaatgataggtttgaattcccagagcagttaccacttgatgaatttttgcaaaaaacagatcctaaggaccctgcaaattatattcttcatgcagtcctggttcatagtggagataatcatggtggacattatgtggtttatctaaaccccaaaggggatggcaaatggtgtaaatttgatgacgacgtggtgtcaaggtgtactaaagaggaagcaattgagcacaattatgggggtcacgatgacgacctgtctgttcgacactgcactaatgcttacatgttagtctacatcagggaatcaaaactgagtgaagttttacaggcggtcaccgaccatgatattcctcagcagttggtggagcgattacaagaagagaaaaggatcgaggctcagaagcggaaggagcggcaggaagcccatctctatatgcaagtgcagatagtcgcagaggaccagttttgtggccaccaagggaatgacatgtacgatgaagaaaaagtgaaatacactgtgttcaaagtattgaagaactcctcgcttgctgagtttgttcagagcctctctcagaccatgggatttccacaagatcaaattcgattgtggcccatgcaagcaaggagtaatggaacaaaacgaccagcaatgttagataatgaagccgacggcaataaaacaatgattgagctcagtgataatgaaaacccttggacaatattcctggaaacagttgatcccgagctggctgctagtggagcgaccttacccaagtttgataaagatcatgatgtaatgttatttttgaagatgtatgatcccaaaacgcggagcttgaattactgtgggcatatctacacaccaatatcctgtaaaatacgtgacttgctcccagttatgtgtgacagagcaggatttattcaagatactagccttatcctctatgaggaagttaaaccgaatttaacagagagaattcaggactatgacgtgtctcttgataaagcccttgatgaactaatggatggtgacatcatagtatttcagaaggatgaccctgaaaatgataacagtgaattacccaccgcaaaggagtatttccgagatctctaccaccgcgttgatgtcattttctgtgataaaacaatccctaatgatcctggatttgtggttacgttatcaaatagaatgaattattttcaggttgcaaagacagttgcacagaggctcaacacagatccaatgttgctgcagtttttcaagtctcaaggttatagggatggcccaggtaatcctcttagacataattatgaaggtactttaagagatcttctacagttcttcaagcctagacaacctaagaaactttactatcagcagcttaagatgaaaatcacagactttgagaacaggcgaagttttaaatgtatatggttaaacagccaatttagggaagaggaaataacactatatccagacaagcatgggtgtgtccgggacctgttagaagaatgtaaaaaggccgtggagcttggggagaaagcatcagggaaacttaggctgctagaaattgtaagctacaaaatcattggtgttcatcaagaagatgaactattagaatgtttatctcctgcaacgagccggacgtttcgaatagaggaaatccctttggaccaggtggacatagacaaagagaatgagatgcttgtcacagtggcgcatttccacaaagaggtcttcggaacgttcggaatcccgtttttgctgaggatacaccagggcgagcattttcgagaagtgatgaagcgaatccagagcctgctggacatccaggagaaggagtttgagaagtttaaatttgcaattgtaatgatgggccgacaccagtacataaatgaagacgagtatgaagtaaatttgaaagactttgagccacagcccggtaatatgtctcatcctcggccttggctagggctcgaccacttcaacaaagccccaaagaggagtcgctacacttaccttgaaaaggccattaaaatccataactgatttccaagctggtgtgttcaaggcgaggacggtgtgtgggtggccccttaacagcctagaactttggtgcacgtgccctctagccgaagtcttcagcaagaggattcgctgctggtgttaattttattttattgaggctgttcagtttggcttctctgtatctattgactgccctttttgagcaaaatgaagatgtttttataaagcttggatgccaatgagagttattttatggtaaccacagtgcaaggcaactgtcagcgcaatgggggagaagaggttagtggatcgggggtccctggctcaaggtctctgggctgtccctagtgggcacgagtggctcggctgccttcctggggtcccgtgcaccagccctgcagctagcaagtcttgtgtttaggctcgtctgacctatttccttcagttatactttcaatgaccttttgtgcatctgttaaggcaaaacagagaaactcacaacctaataaatagcgctcttcccttcattgtgtgcattgtcggcccttcctcgggttctcctcctccagctgcctgggggctttttaataaacttgtctcacctcgtcagccactactgtctgcagcccctttgcaaagtggatgcactgaatacagtccggacagacattgtgggggtctttttattaaatcaagaacattgttaaattcaattaaggtttactctgctgccttggcagacttacgatctcaacagttcatacgagcaggtgaaaggattataaatagaatttcgttaaagtggaacagacgacaagaaagccttttagcaagagggcatgctcactagtggttagtaagctgtcgactttgtaaaaaagttaaaaatgaaaaaaaaaggaaaaatgaattgtatatttaatgaatgaacatgtacaatttgccactgggaggaggttcctttttgttgggtgagtctgcaagtgaatttcactgatgttgatattcattgtgtgtagttttatttcggtcccagccccgtttccttttattttggagctaatgccagctgcgtgtctagttttgagtgcagtaaaatagaatcagcaaatcactcttatttttcatccttttccggtattttttgggttgtttctgtgggagcagtgtacaccaactcttcctgtatattgcctttttgctggaaaatgttgtatgttgaataaaattttctataaaaattataattcagtgagttacgtggaagtggaggaagatttctactctccctggaaacaggcctgggaaaccttggcatttgtaacaaggtttcactgagatgtacttttccttctaattccgttttgcgggggcagggtctcttgtttctttttttttttttttttttttttagcctctaactagtcacatttactcttaagaaatgaaaggttttccaggagagaactgtgtacaaataaggtgactggagatgtgacctgatgtgtcacgaggcccttcggggcggcaggcgctatcgtgggcgtggtccttgcaccgtcccatcggccttgccttccagctccgtggcacggtttcctggtctttgggccagtgtgtaccttggagtgacttcctttctcaacttccactgcagtgtgtgtgccttctgctctgagagctgccttgtgacccgtgtgatagaaagcagggagtgagggtccccgcggacctggcccttccctccttcctcccccagaaagaggagttagagcaggggtgcgagagccgttcgctgtgggtttgtctttgaacaaacattaaggtgtcttgtttttgttctgggctgggggttggctgtagtcttaggtaactgaaagttcctactctcccttaaggtattaaatgactctttttccaaa

SEQ ID NO: 2 - protein, 1102 aa, Homo sapiens
MNHQQQQQQQKAGEQQLSEPEDMEMEAGDTDDPPRITQNPVINGNVALSDGHNTAEEDMEDDTSWRSEATFQFTVERFSRLSESVLSPPCFVRNLPWKIMVMPRFYPDRPHQKSVGFFLQCNAESDSTSWSCHAQAVLKIINYRDDEKSFSRRISHLFFHKENDWGFSNFMAWSEVTDPEKGFIDDDKVTFEVFVQADAPHGVAWDSKKHTGYVGLKNQGATCYMNSLLQTLFFTNQLRKAVYMMPTEGDDSSKSVPLALQRVFYELQHSDKPVGTKKLTKSFGWETLDSFMQHDVQELCRVLLDNVENKMKGTCVEGTIPKLFRGKMVSYIQCKEVDYRSDRREDYYDIQLSIKGKKNIFESFVDYVAVEQLDGDNKYDAGEHGLQEAEKGVKFLTLPPVLHLQLMRFMYDPQTDQNIKINDRFEFPEQLPLDEFLQKTDPKDPANYILHAVLVHSGDNHGGHYVVYLNPKGDGKWCKFDDDVVSRCTKEEAIEHNYGGHDDDLSVRHCTNAYMLVYIRESKLSEVLQAVTDHDIPQQLVERLQEEKRIEAQKRKERQEAHLYMQVQIVAEDQFCGHQGNDMYDEEKVKYTVFKVLKNSSLAEFVQSLSQTMGFPQDQIRLWPMQARSNGTKRPAMLDNEADGNKTMIELSDNENPWTIFLETVDPELAASGATLPKFDKDHDVMLFLKMYDPKTRSLNYCGHIYTPISCKIRDLLPVMCDRAGFIQDTSLILYEEVKPNLTERIQDYDVSLDKALDELMDGDIIVFQKDDPENDNSELPTAKEYFRDLYHRVDVIFCDKTIPNDPGFVVTLSNRMNYFQVAKTVAQRLNTDPMLLQFFKSQGYRDGPGNPLRHNYEGTLRDLLQFFKPRQPKKLYYQQLKMKITDFENRRSFKCIWLNSQFREEEITLYPDKHGCVRDLLEECKKAVELGEKASGKLRLLEIVSYKIIGVHQEDELLECLSPATSRTFRIEEIPLDQVDIDKENEMLVTVAHFHKEVFGTFGIPFLLRIHQGEHFREVMKRIQSLLDIQEKEFEKFKFAIVMMGRHQYINEDEYEVNLKDFEPQPGNMSHPRPWLGLDHFNKAPKRSRYTYLEKAIKIHN

SEQ ID NO: 3 - DNA, 5315 bp, Homo sapiens
gtacgtgtgtgtgtccacatctttgagtgccgggagtttaaaagttaggcagtccttataggtatggaagccgagctaatttccttctgagccccccaaatgcctcctccacatggcgggtccgcaaatggggggatctgcagaggatcaccccccacgaaaaagacatgcagcagaaaagcaaaaaaagaaaacagttatatatacaaaatgctttgaatttgagtcggccacacagcgcccgattgaccgccagcgatacgacgagaacgaggacttgtcggacgtggaggagatcgtcagcgtccgcggcttcagcctggaggagaagcttcgcagccagctgtaccagggggacttcgtgcacgccatggagggcaaagatttcaactatgagtacgtacagagagaagctctcagggttcccctgatatttcgagaaaaggatggactgggaattaagatgcctgaccctgatttcacagtccgagacgtcaaactcctagtggggagccggcggcttgtggacgtgatggatgtgaacacccagaagggcacggagatgagcatgtcccagtttgtgcgttactacgagacgcccgaggcccagcgggacaagctgtacaacgtcatcagcctagagttcagccacaccaagctggagcacttggtcaagcgtccgactgtggtagacctggtggactgggtggacaacatgtggccccagcatctgaaggagaagcagacagaagccacgaacgccattgcagagatgaagtacccgaaagtgaaaaagtactgtctgatgagcgtgaaaggttgtttcaccgacttccacatcgactttggaggcacttccgtttggtaccatgttttccggggtgggaagattttttggctgattcctccaacgctgcacaatttggcgctgtacgaggagtgggtgctgtcaggcaaacagagtgacatctttctgggagaccgtgtggaacgatgccaaagaattgagctgaagcagggctacacatttttcatcccttccggttggatccatgccgtctacacccctgtagactctttggtgttcggcggaaacatcctgcacagctttaacgtgcccatgcagctgcggatctacgagatcgaggacaggacgcgggtgcagcccaaattccgttaccccttctactatgagatgtgctggtatgtcctggagagatacgtgtactgtgtgacccagcgctcccacctcactcaggaataccagagggagtcgatgcttattgatgccccgaggaagcccagcatagacggcttctcttcggattcctggctggagatggaggaggaggcctgtgatcagcagcctcaggaggaggaggagaaggacgaggagggcgagggcagggacagggcacccaaaccgcccaccgatggctccacttcacccaccagcacgccctctgaggaccaggaggccctcgggaagaagcccaaagcacctgccctgcgattcctcaaaaggactttgtctaatgagtcggaggaaagtgtgaagtccaccacattggccgtagactaccccaagacccccaccggctctcccgccacggaggtctctgccaaatggacccatctcactgagtttgaactgaagggcctgaaagctctggtggagaaactggaatccctcccggagaacaagaagtgtgtccccgagggcatcgaggacccccaggcactcctggagggtgtgaagaacgtcctgaaggagcacgcagatgatgaccctagtctggccatcactggggtccctgtggtgacttggccaaagaagactccaaagaaccgggctgtgggtcggcccaaggggaagctgggcccggcctccgcggtgaagttggccgccaaccggacaacggcaggagctcggcggcgccggacgcgatgccgcaagtgcgaggcctgcctgcggaccgagtgcggagagtgccacttctgcaaggacatgaagaagttcgggggccccgggcgcatgaagcagagctgcatcatgcggcagtgcatcgcgccagtgctgccccacaccgccgtgtgccttgtgtgtggcgaggcggggaaggaagacacggtggaagaggaggaaggcaagtttaacctcatgctcatggagtgctccatctgcaatgaaatcatccaccctggatgccttaagattaaggagtcagagggtgtggtcaacgacgagcttccaaactgctgggagtgtccgaagtgtaaccacgccggcaagaccgggaaacaaaagcgtggccctggctttaagtacgcctccaacctgcccggctccctgctcaaggagcagaagatgaaccgggacaacaaggaagggcaggaacctgccaagcggaggagtgagtgtgaggaggcgccccggcgcaggtcggatgagcactcgaagaaggtgccgccggacggccttctgcgcagaaagtctgacgacgtgcacctgaggaagaagcggaaatacgagaagccccaggagctgagtggacgcaagcgggcctcatcgcttcaaacgtcccccggttcctcctctcacctctcgccgaggccccctctaggcagcagcctcagcccctggtggagatccagtctcacttacttccagcagcagctcaaacctggcaaagaagataagcttttcaggaaaaagcggcggtcctggaagaacgccgaggaccgcatggcgctggccaacaagcccctccggcgcttcaagcaggaacccgaggacgaactgcccgaggcgccccccaagaccagggagagcgaccactcccgctccagctcccccaccgcgggacccagcaccgaaggggccgagggcccggaggagaagaagaaggtgaagatgcgccggaagcggcggcttcccaacaaggagctgagcagggagctgagcaaggagctcaaccacgagatccagaggacggagaacagcctggccaacgagaaccagcagcccatcaagtcggagcctgagagcgagggcgaggagcccaagcggcccccgggcatctgcgagcgtccccaccgcttcagcaaggggctcaacggcaccccccgggagctgcggcaccagctggggcccagcctgcgcagcccgccccgtgtcatctcccggcccccaccctccgtgtccccgcccaagtgtatccagatggagcgccatgtgatccggccaccccccatcagccccccgcctgactcgctacccctggacgatggggcagcccacgtcatgcacagggaggtgtggatggccgtcttcagctacctcagccaccaagacctgtgtgtgtgcatgcgggtctgcaggacctggaaccgctggtgctgcgataagcggttgtggacccgcattgacctgaaccactgcaagtctatcacacccctgatgctgagtggcatcatccggcgacagcccgtctccctcgacctcagctggaccaatatctccaagaagcagctgagctggctcatcaaccggctgcctgggctccgggacttggtgctgtcaggctgctcatggatcgcggtctcggccctttgcagctccagttgtccgctgctccggaccctggatgtccagtgggtggagggactaaaggatgcccagatgcgggatctcctgtccccgcccacagacaacaggccaggtcagatggacaatcggagcaagctccggaacatcgtggagctgcgcctggcaggcctggacatcacagatgcctccctgcggctcatcatccgccacatgcccctgctctccaagctccacctcagttactgtaaccacgtcaccgaccagtctatcaacctgctcactgctgttggcaccaccacccgagactccttaaccgagatcaacctgtctgactgcaataaggtcactgatcagtgcctgtccttcttcaaacgctgtggaaacatctgtcatattgacctgaggtactgcaagcaagtcaccaaggaaggctgtgagcagttcatagccgagatgtctgtgagtgtccagtttgggcaagtagaagaaaaactcctgcaaaaactgagttagtccaaggataagtatgtaaatacggggcgggctctgggaggggagagactttacaaaaatgagggcttttattttccatttggaacgtgggacaacagaccacaacgcaattccattttgcaagtctttccaagggagaagctgttcaaccacccgtttgggggatgagtgagccgacactttcctttggtctttctgaatcgtaactgcactgctttctggaccatttctaaggcggcctttacaagaagacattcctgtcggagaggagggtggacttcggagaaattctcatactgaagcatgagcttaggagtttctgttagtggtagtggtgttttggacacttcattccttgcaacaccgaggttttgggtgttgacataaagtggaccacacaccacatctgctgccgtcttgacacttttttttgtttggttggttttgttacatcttacattatgcagaactatttttgtacaaattgtttaaaagttatttatgcaaggtttgaatgcataccagtgtttttattgttttgagattgccaattttcctgatttccttaaggtaggagagaatttaacgtgtacttcatcgacacaacccatctacaaatgtgcccagatctaacaaagtaggctaagaccttccacttaaaagcatgtttaactggaagttgagagtctgctttgtacctcaagagttacatgagcatgttgtggataaatgtaaattatagtcaaagtaagatactctgccaagtttcctctgtagagaattcacttttctcaaattttaaaatttcgacttcagcctttgcactcaggaggttctgctccagcatgagctcttgtacttacatagatctaatttatacagtgagtcaagacgtagaataaatgctcccacatagcctttcttttgcttttgcttctctcctctgaagtgtgagttgagttctcatttaggtttgtaacatggctatttcctagttgtaaagttctgcatttataagtgccattgttgtaaggtggtgtttcctagaccttccctgatgcgattttacctttgttgaatttgtataaacaattgtacaaaaaaaaccactcttgaactttgagggtttctgttctaggagtggactagaagtttaagcccagagtcagtaaacactgttttgaagtccaaa

SEQ ID NO: 4 - protein, 1336 aa, Homo sapiens
MAGPQMGGSAEDHPPRKRHAAEKQKKKTVIYTKCFEFESATQRPIDRQRYDENEDLSDVEEIVSVRGFSLEEKLRSQLYQGDFVHAMEGKDFNYEYVQREALRVPLIFREKDGLGIKMPDPDFTVRDVKLLVGSRRLVDVMDVNTQKGTEMSMSQFVRYYETPEAQRDKLYNVISLEFSHTKLEHLVKRPTVVDLVDWVDNMWPQHLKEKQTEATNAIAEMKYPKVKKYCLMSVKGCFTDFHIDFGGTSVWYHVFRGGKIFWLIPPTLHNLALYEEWVLSGKQSDIFLGDRVERCQRIELKQGYTFFIPSGWIHAVYTPVDSLVFGGNILHSFNVPMQLRIYEIEDRTRVQPKFRYPFYYEMCWYVLERYVYCVTQRSHLTQEYQRESMLIDAPRKPSIDGFSSDSWLEMEEEACDQQPQEEEEKDEEGEGRDRAPKPPTDGSTSPTSTPSEDQEALGKKPKAPALRFLKRTLSNESEESVKSTTLAVDYPKTPTGSPATEVSAKWTHLTEFELKGLKALVEKLESLPENKKCVPEGIEDPQALLEGVKNVLKEHADDDPSLAITGVPVVTWPKKTPKNRAVGRPKGKLGPASAVKLAANRTTAGARRRRTRCRKCEACLRTECGECHFCKDMKKFGGPGRMKQSCIMRQCIAPVLPHTAVCLVCGEAGKEDTVEEEEGKFNLMLMECSICNEIIHPGCLKIKESEGVVNDELPNCWECPKCNHAGKTGKQKRGPGFKYASNLPGSLLKEQKMNRDNKEGQEPAKRRSECEEAPRRRSDEHSKKVPPDGLLRRKSDDVHLRKKRKYEKPQELSGRKRASSLQTSPGSSSHLSPRPPLGSSLSPWWRSSLTYFQQQLKPGKEDKLFRKKRRSWKNAEDRMALANKPLRRFKQEPEDELPEAPPKTRESDHSRSSSPTAGPSTEGAEGPEEKKKVKMRRKRRLPNKELSRELSKELNHEIQRTENSLANENQQPIKSEPESEGEEPKRPPGICERPHRFSKGLNGTPRELRHQLGPSLRSPPRVISRPPPSVSPPKCIQMERHVIRPPPISPPPDSLPLDDGAAHVMHREVWMAVFSYLSHQDLCVCMRVCRTWNRWCCDKRLWTRIDLNHCKSITPLMLSGIIRRQPVSLDLSWTNISKKQLSWLINRLPGLRDLVLSGCSWIAVSALCSSSCPLLRTLDVQWVEGLKDAQMRDLLSPPTDNRPGQMDNRSKLRNIVELRLAGLDITDASLRLIIRHMPLLSKLHLSYCNHVTDQSINLLTAVGTTTRDSLTEINLSDCNKVTDQCLSFFKRCGNICHIDLRYCKQVTKEGCEQFIAEMSVSVQFGQVEEKLLQKLS

SEQ ID NO: 5 - DNA, 7243 bp, Homo sapiens
actctttcgctcgccgcggctgctgccagtgtgtggctctgtctctcctccgctttgctgagccctcccttcttcctctcagttcctagagtccgaccgccgccgccgccgagagagaggagaaggagggggagtggccacagcaggtcctatctggtggtgagtggctgtcatgatctctacagcaccgctctacagcggcgtgcacaactggaccagttctgaccggattcgcatgtgtggcatcaacgaggagagaagagcacctctttctgatgaggagtcaacgacaggcgactgccagcactttggatctcaggagttttgtgtcagcagcagtttttccaaggtggagctcacggcagttggaagtggcagcaatgcccggggggcagacccagatggcagtgctacagaaaaacttgggcacaagtcagaagacaagcctgacgatccccagccaaaaatggactacgctgggaacgtggcagaggctgagggcctcttggtgcccctgagcagcccaggagacgggctcaagcttcccgcatctgacagcgccgaggccagcaacagcagggccgactgctcctggactccactcaacacccaaatgagcaaacaggttgactgctcacccgccggagtaaaggctttggactctcggcaaggtgttggagagaagaatactttcattttggcaactctgggaactggagtccctgtggaggggaccctgcccctggttaccactaacttcagtcctctgccagcccctatctgtccccctgctcccggttcggcctctgtgccccactctgttccagatgcattccaggttcccctctccgtccctgccccagtcccccattcagggcttgttccagtccaagttgccacttcggttccagctccttcccctcccttagcacctgtcccggctctggctccagcgccaccgtcagtgcccacgctcatctctgactcgaaccccctttctgtttcggcctcagtcttggtgcctgtgccagcttctgctcccccttcaggcccggttcccttgtcggctccagctcctgccccgctttcagtcccagtttcagctcctcccttggctctcatccaggctcctgtgcccccttcagctccgaccttggttctcgctcccgtccccactccggttctggctcccatgccagcatccacgcctccagcggcccctgcccctccgtctgtgcccatgcccactccaaccccatcttccggcccaccttctacccccaccctcatccccgcctttgctcctacaccggtgcctgcacccaccccagcccccatctttactccagcccctacacccatgcctgctgccacgccagctgccattcccacctctgcacccatcccggcctccttcagtttgagtagagtgtgctttcctgcagctcaggcaccagctatgcaaaaagtccccctgtcctttcagccagggacagtgctgaccccgagccagccgctggtatatatcccgcctccaagctgtgggcagccactcagtgtggccacactgccaaccactctaggggtttcctccactcttacgctccctgtcctgccgtcctacctgcaggacaggtgtctcccaggcgtgctagcctcccccgagctccgttcttacccgtatgcattttctgtggcccggcctctgacttcggattccaagctggtatctctggaggtgaacaggctcccctgcacttccccatccggtagcaccaccacccagcctgcacccgatggggtccctgggcctttggcagatacctcccttgttactgcttctgccaaggtgcttccaactccacagcctctgctgccagcccccagtgggagctcagccccaccgcaccccgccaagatgcccagtggcaccgagcagcaaacagaagggacttccgttaccttctctcctcttaagtcaccgccacagctggaacgagagatggcctctccacctgagtgcagcgagatgccccttgatctgtcctccaagtccaaccgccagaagcttccattgccgaaccagcgcaagacaccccccatgcctgtgttgacccccgtgcacaccagcagcaaggccctcctctccacagtcctgtctaggtctcagcgcacaacccaggctgccggtggcaatgtcacctcctgcctgggctccacttcctcgccctttgtcatctttcccgagatcgtgaggaatggggacccgagcacctgggtgaagaactcaactgcactgatcagcaccattcctggcacctacgtgggagtggccaacccagtgcctgcatccctgctgctgaacaaagaccccaacctgggcctcaaccgtgacccccgccatctccccaagcaggagcccatctccatcattgatcaaggagagcctaagggcactggtgccacgtgtggcaaaaagggcagccaggctggtgctgagggacagccaagcacagtgaaacgatatactccagcccgcattgcccctgggctgccagggtgccaaaccaaggaactctctttgtggaaacccacggggccggcaaatatttatccccggtgttcagtcaatgggaaacctaccagcacccaggtcctgcctgttggctggtccccgtaccaccaggcgtctctgctttccattggcatttccagtgccgggcagctgacccccagtcagggggcgcccatcaggcccaccagcgttgtttcggagttttctggtgtgccatctctcagctccagcgaagccgtgcacggacttcctgaggggcaaccacggcctgggggctccttcgttccagagcaggaccctgttacaaagaacaaaacttgccggattgctgccaagccttatgaagaacaagtcaatcctgtcctcttgaccctcagccctcagactgggaccctggcactgtctgttcagcctagcggtggggacattcgaatgaatcaggggcctgaggaatcagagagccacctctgctctgacagcactcctaagatggaaggcccccagggggcttgtggcctgaagctggcaggagacacgaagcctaagaaccaagtgctggccacctacatgtcccatgagctggtcctggccaccccccagaacctgcctaagatgcctgagctgcctttgctacctcacgacagccaccccaaggaacttatattggacgtggttccgagcagcaggaggggctccagcacagagcgcccacagcttggaagccaggtggatctggggcgagtgaaaatggagaaggtggatggtgatgtggtcttcaatttagccacctgcttccgggctgatggcctcccagtggctccccagaggggccaagctgaagttcgggctaaggccgggcaggctcgagtgaaacaggaaagcgtaggggtctttgcttgcaagaacaagtggcagccagatgatgtgacggaatctctgccgcccaagaagatgaagtgcggcaaagagaaggacagtgaagagcagcagctccagccacaagccaaggccgtggtccggagttcccacagacccaagtgccggaagctgcccagtgacccccaggaatccaccaagaaaagccccaggggggcttcagattcaggaaaagagcacaatggagtcaggggaaagcacaagcaccggaagccgacaaagccggagtcccagtctccaggaaaacgagccgacagccacgaggaaggttccttggaaaagaaagcaaagagcagtttccgtgactttattcctgtggttctgagcacccgcacgcgcagtcagtctggaagcatctgtagctcctttgctggcatggcagacagtgacatgggaagccaggaagtcttccccacagaagaagaagaggaggtaacccccaccccagctaagcgtcgaaaggtgagaaagacccaacgggacacccagtatcgcagccaccatgcccaggacaagtctctgctgagccagggccgaaggcacctgtggcgagcccgagaaatgccctggaggacagaggctgcccggcaaatgtgggacaccaatgaggaggaggaggaagaagaggaggagggcctgctgaagaggaagaaacgaagacggcagaagagccgaaaatatcagactggggagtacctgacagagcaagaagacgagcagcggcggaaagggagagcagatttaaaggcccgtaagcagaagacttcctcctcccaaagtttggagcaccgcctcaggaacaggaaccttctcttgcccaacaaagtccaggggatctcggattcaccaaacggtttcctcccaaataacctggaagagccagcctgccttgaaaattcagaaaagccatcaggaaaacgaaagtgcaagaccaagcacatggcaaccgtctcagaagaggcaaaggatgttgttctctactgcctccagaaagacagtgaagatgtgaatcaccgtgacaatgctggctacacagccctgcatgaggcttgttcccggggctggaccgacatcctgaacatcctgctggagcacggggccaacgtgaactgcagtgcgcaggacggcacgaggccagttcatgatgcggtggtcaatgacaacctggagaccatctggctcctgctgtcctatggggccgatcccacactggctacctactcgggtcagacagccatgaagctggccagcagcgacaccatgaagcgctttctcagtgatcacctctcggatcttcagggccgggcagagggtgatcccggtgtatcctgggatttttacagcagttctgtgttggaggaaaaagacgggtttgcctgtgacctcctacataatcctcctgggagctcagatcaagaaggagacgatccgatggaggaggatgatttcatgtttgaactctcagacaagcctcttctcccttgctacaacctccaagtgtcagtgtcccgcgggccctgcaactggttcctcttttccgatgtcttgaagaggctgaagctttcctcgaggatctttcaggcccggttcccgcactttgaaatcaccaccatgcccaaggccgagttctacaggcaggtggcctccagtcagctgctgacccctgccgagaggcctggaggcttggacgacagatcccccccaggctcctctgagactgtggagctggtgcggtacgagccagacctacttcggctcctagggtccgaggtggaattccagtcttgcaacagttgaccgggaaaacagcccctcctcttctttctccttccgagttcgcccttcccccacctccttgtctttccccgaccgagcaccagactgcagaatgaggcaataatacggaccaacaagaagccgccttatcaatgccagcattagcgactggactgtttttgtttttttggttacaattagttctcatctccctgtcgtcgtcattgttatcgtggttgctgatgggggtggaaagttgaactccatgtctgaggacaagaggtcccgggggtggtgggaggtggcgccggggtcccttggactggcctccttgttcatgaccaagaccaaacctgggccctggatggccttggcctgtcccgaggagaaatgagaaaatcccagatctctgagcgccccccaactccattcccctgtgttcttctgtcttctgtagtatttattttattagtatttaatttgtattgtttcattggtttctgataagtctgtatcactgtgacgatttgagacaacttgttgtattgagggactttctgtacctccttttctttttctttgttgatgagctctgacaaagctattccctggtgtttttttcccccactggggagggggtgaggtggaatggggtgggggaacatggacttgtgactaacgaagctggttgctgctggcccagggctgggggcttgggggtaaatcctgaggctttggtgctcccccacccacccattcccgccctttgcagcagccccgctatcttgagattagtgttgacagggaggggaggattgtgaggtgaggggttaataagttactctaataaaggagcgtggagaagggatctgaggggtgagggtggcccccctcctcacgccttcttcactgcccccctcagagtgcacaatacgagtttgttcctgcctccactctcccaccccgttctggcctccctgtctcaagatactgagcctctcacctcccagccctcagccacccccatccctgccccttctgagactcacagcacccctttccttcctctcctcccacctcctccctcagcccctcattctccttgggaatctgcagagggctctgggactcactgccggatgtgaaatccaggcgtcagctgtttcctaggcaagggcaggaaagtggtctccagcccttgctccactcatgcctgggggcctggggctgagtggtatccctacctggcctccccctggcctctgggcctccagcgctgggtttgtcgagtgagagagagagaggagcttgggttgcttccctgtccccgccccctctgtggcattgtccctcccactcttatttttctaccaattgctatttttccgaacaatccttgtagagtatgtaccatccaaaggcaggagggcctcgccgtggccggctctggttggagatggtacagttttattgtacaggtgctaaaacaacaacaacaaaaaagaaaatggaaaaaaaaaagattaaaaaaaaaaggaaaaaaaaaaagccagtttgaggatgggacaatctgttctctagaggctcctgagccatgcgggagcattggtggttattttctttgtattgtgtttgttctttgttcctgggggggaagttctcggcccccttctgtaggactgctccccacccccaccatactgcccagttggttttgaacagttgttttccctttttaagaaaaaaaaatacatatatatatacatatatatatataaagttgaggggttttggactttaatttgttggttttgttggggttcctggtattgtgtagtttatttcatgttctgtttgcctttccttttttcgcatttgggtgtatattctggctgccctttatgtttcattttaagcaactggctgtggagtcaaaaacacttgcatactgaaaaa

SEQ ID NO: 6 - protein, 1711 aa, Homo sapiens
MISTAPLYSGVHNWTSSDRIRMCGINEERRAPLSDEESTTGDCQHFGSQEFCVSSSFSKVELTAVGSGSNARGADPDGSATEKLGHKSEDKPDDPQPKMDYAGNVAEAEGLLVPLSSPGDGLKLPASDSAEASNSRADCSWTPLNTQMSKQVDCSPAGVKALDSRQGVGEKNTFILATLGTGVPVEGTLPLVTTNFSPLPAPICPPAPGSASVPHSVPDAFQVPLSVPAPVPHSGLVPVQVATSVPAPSPPLAPVPALAPAPPSVPTLISDSNPLSVSASVLVPVPASAPPSGPVPLSAPAPAPLSVPVSAPPLALIQAPVPPSAPTLVLAPVPTPVLAPMPASTPPAAPAPPSVPMPTPTPSSGPPSTPTLIPAFAPTPVPAPTPAPIFTPAPTPMPAATPAAIPTSAPIPASFSLSRVCFPAAQAPAMQKVPLSFQPGTVLTPSQPLVYIPPPSCGQPLSVATLPTTLGVSSTLTLPVLPSYLQDRCLPGVLASPELRSYPYAFSVARPLTSDSKLVSLEVNRLPCTSPSGSTTTQPAPDGVPGPLADTSLVTASAKVLPTPQPLLPAPSGSSAPPHPAKMPSGTEQQTEGTSVTFSPLKSPPQLEREMASPPECSEMPLDLSSKSNRQKLPLPNQRKTPPMPVLTPVHTSSKALLSTVLSRSQRTTQAAGGNVTSCLGSTSSPFVIFPEIVRNGDPSTWVKNSTALISTIPGTYVGVANPVPASLLLNKDPNLGLNRDPRHLPKQEPISIIDQGEPKGTGATCGKKGSQAGAEGQPSTVKRYTPARIAPGLPGCQTKELSLWKPTGPANIYPRCSVNGKPTSTQVLPVGWSPYHQASLLSIGISSAGQLTPSQGAPIRPTSVVSEFSGVPSLSSSEAVHGLPEGQPRPGGSFVPEQDPVTKNKTCRIAAKPYEEQVNPVLLTLSPQTGTLALSVQPSGGDIRMNQGPEESESHLCSDSTPKMEGPQGACGLKLAGDTKPKNQVLATYMSHELVLATPQNLPKMPELPLLPHDSHPKELILDVVPSSRRGSSTERPQLGSQVDLGRVKMEKVDGDVVFNLATCFRADGLPVAPQRGQAEVRAKAGQARVKQESVGVFACKNKWQPDDVTESLPPKKMKCGKEKDSEEQQLQPQAKAVVRSSHRPKCRKLPSDPQESTKKSPRGASDSGKEHNGVRGKHKHRKPTKPESQSPGKRADSHEEGSLEKKAKSSFRDFIPVVLSTRTRSQSGSICSSFAGMADSDMGSQEVFPTEEEEEVTPTPAKRRKVRKTQRDTQYRSHHAQDKSLLSQGRRHLWRAREMPWRTEAARQMWDTNEEEEEEEEEGLLKRKKRRRQKSRKYQTGEYLTEQEDEQRRKGRADLKARKQKTSSSQSLEHRLRNRNLLLPNKVQGISDSPNGFLPNNLEEPACLENSEKPSGKRKCKTKHMATVSEEAKDVVLYCLQKDSEDVNHRDNAGYTALHEACSRGWTDILNILLEHGANVNCSAQDGTRPVHDAVVNDNLETIWLLLSYGADPTLATYSGQTAMKLASSDTMKRFLSDHLSDLQGRAEGDPGVSWDFYSSSVLEEKDGFACDLLHNPPGSSDQEGDDPMEEDDFMFELSDKPLLPCYNLQVSVSRGPCNWFLFSDVLKRLKLSSRIFQARFPHFEITTMPKAEFYRQVASSQLLTPAERPGGLDDRSPPGSSETVELVRYEPDLLRLLGSEVEFQSCNS

SEQ ID NO: 7 - DNA, 1739 bp, Homo sapiens
cgggccatggcggcggcggtggcgggagctgctgtctgagcagcggttgcggaccgagcgaacttggcccaggagcccgggcctagggagaggcgcggcggcggcgggagcgcgaacggctggagctggccttcttcgccttctcctcggctgtggagccctggtggggggtctgcgcccggtcaccatgacgacgccggcgaatgcccagaatgccagcaaaacgtgggaactgagtctgtatgagctgcaccggaccccgcaggaagccataatggatggcacagagattgctgtttcccctcggtcactgcattcagaactcatgtgccctatctgcctggacatgctgaagaatacgatgaccaccaaggagtgcctccacagattctgctctgactgcattgtcacagccctacggagcgggaacaaggagtgtcctacctgccgaaagaagctggtgtccaagcgatccctacggccagaccccaactttgatgccctgatctctaagatctatcctagccgggaggaatacgaggcccatcaagaccgagtgcttatccgcctgagccgcctgcacaaccagcaggcattgagctccagcattgaggaggggctacgcatgcaggccatgcacagggcccagcgtgtgaggcggccgataccagggtcagatcagaccacaacgatgagtgggggggaaggagagcccggggagggagaaggggatggagaagatgtgagctcagactccgcccctgactctgccccaggccctgctcccaagcgaccccgtggagggggcgcaggggggagcagtgtagggacagggggaggcggcactggtggggtgggtgggggtgccggttcggaagactctggtgaccggggagggactctgggagggggaacgctgggccccccaagccctcctggggcccccagccccccagagccaggtggagaaattgagctcgtgttccggccccaccccctgctcgtggagaagggagaatactgccagacgaggtatgtgaagacaactgggaatgccacagtggaccacctctccaagtacttggccctgcgcattgccctcgagcggaggcaacagcaggaagcaggggagccaggagggcctggagggggcgcctctgacaccggaggacctgatgggtgtggcggggagggtgggggtgccggaggaggtgacggtcctgaggagcctgctttgcccagcctggagggcgtcagtgaaaagcagtacaccatctacatcgcacctggaggcggggcgttcacgacgttgaatggctcgctgaccctggagctggtgaatgagaaattctggaaggtgtcccggccactggagctgtgctatgctcccaccaaggatccaaagtgaccccaccaggggacagccagaggaaggggaccatggggtatccctgtgtcctggtctatcaccccagcttctttgtcccccagtacccccagcccagccagccaataagaggacacaaatgaggacacgtggcttttatacaaagtatctatatgagattcttctatattgtacagagtggggcaaaacacgcccccatctgctgccttttctattgccctgcaacgtcccatctatacgaggtgttggagaaggtgaagaaccctcccattcacgcccgcctaccaacaacaaacgtgcttttttcctctttgaaaaaaaaaaaaaaaaa

SEQ ID NO: 8 - protein, 406 aa, Homo sapiens
MTTPANAQNASKTWELSLYELHRTPQEAIMDGTEIAVSPRSLHSELMCPICLDMLKNTMTTKECLHRFCSDCIVTALRSGNKECPTCRKKLVSKRSLRPDPNFDALISKIYPSREEYEAHQDRVLIRLSRLHNQQALSSSIEEGLRMQAMHRAQRVRRPIPGSDQTTTMSGGEGEPGEGEGDGEDVSSDSAPDSAPGPAPKRPRGGGAGGSSVGTGGGGTGGVGGGAGSEDSGDRGGTLGGGTLGPPSPPGAPSPPEPGGEIELVFRPHPLLVEKGEYCQTRYVKTTGNATVDHLSKYLALRIALERRQQQEAGEPGGPGGGASDTGGPDGCGGEGGGAGGGDGPEEPALPSLEGVSEKQYTIYIAPGGGAFTTLNGSLTLELVNEKFWKVSRPLELCYAPTKDPK

SEQ ID NO: 9 - DNA, 3407 bp, Homo sapiens
atattgtgcggcggcgccggcgtccgcggcagctgataccagagtcttgctccggccgcggccagcggagccctgggctggggcaggagccgcaatgtctcaggctgtgcagacaaacggaactcaaccattaagcaaaacatgggaactcagtttatatgagttacaacgaacacctcaggaggcaataacagatggcttagaaattgtggtttcacctcgaagtctacacagtgaattaatgtgcccaatttgtttggatatgttgaagaacaccatgactacaaaggagtgtttacatcgtttttgtgcagactgcatcatcacagcccttagaagtggcaacaaagaatgtcctacctgtcggaaaaaactagtttccaaaagatcactaaggccagacccaaactttgatgcactcatcagcaaaatttatccaagtcgtgatgagtatgaagctcatcaagagagagtattagccaggatcaacaagcacaataatcagcaagcactcagtcacagcattgaggaaggactgaagatacaggccatgaacagactgcagcgaggcaagaaacaacagattgaaaatggtagtggagcagaagataatggtgacagttcacactgcagtaatgcatccacacatagcaatcaggaagcaggccctagtaacaaacggaccaaaacatctgatgattctgggctagagcttgataataacaatgcagcaatggcaattgatccagtaatggatggtgctagtgaaattgaattagtattcaggcctcatcccacacttatggaaaaagatgacagtgcacagacgagatacataaagacttctggtaacgccactgttgatcacttatccaagtatctggctgtgaggttagctttagaagaacttcgaagcaaaggtgaatcaaaccagatgaaccttgatacagccagtgagaagcagtataccatttatatagcaacagccagtggccagttcactgtattaaatggctctttttctttggaattggtcagtgagaaatactggaaagtgaacaaacccatggaactttattacgcacctacaaaggagcacaaatgagcctttaaaaaccaattctgagactgaacttttttatagcctatttctttaatattaaagatgtactggcattacttttatggacagatcttggatatgttgttcaattttctttctgagccagactagtttacgctattcaaatcttttcccctttatttaagatttcctttttggaagggactgcaattattcagtatttttttctttcctttaaaaaaatatatctgaagtttcttgtgtttttttttttccccacaaagtgtgtttccacttggagcaccattttgacccaggaatttttcatagtttctgtattcttataagattcagttggctgtccttttcctgctcccctcaaaagatttttagtcatacagaatgttaaatattatgtattctgactttttttttcccccggagtcttgtatatttatagttttctatataaactgtagtatcttcatgaagacccaaggctcaaatttactgtccttaaaaacaattctcataggattattcttttcatggtattttcttccataatatctcattttaaaaagaagttctttatgaacttagtgtccattgtcatgcaatgtttttttttttccattctttttccctgtaattttggaatttctggtcctgggaagaatcaaacaaaatcttaagttctatgagaacttggttcattgacatattctgctgaagaaagaaaaattaaattggtagtaaaatatagtcttcaagtatacgtttgagagtgcttttttttgtattagttctgctgtcacttcatttcctgtattatatgtgatgtttttccccattaaaataccagagataatggagatattttgcactttagccttgatgaaaagtacaagatatgttcaaagcttccctaatttttttcttatttgtagccacataagtttcaagaataacatggcacacagaacaatggaaaaaagtttgtttccattggaaaattatatcattttgggttgccacatcagtttataaatttggcgctcttttaattacactctgtagaaggttaatagagcttgagccctgctttaatatgtagtgaaagataattctgtagaaaaacgtcagccagtagggtaaagtcattctactgttcttaatttttatattgaggaacaatattgggtgtttgggagccagaaagctttgttgacagatcagaaataagattgacttgggtgttatatttcatctctctccagactctaggtatatttccaactttatatatcacagtatttaaaaagacatgtttgcattgagaaattaaccctaaagggttttcaatagggtgtagacctccagtacctttgtaactaaagtctgtctagtcattgtaaatatttatctgtcagttttgacagattggggccagcttgatgttttaaatcttcagcccggtatgaaaacttaaaggtatatattcaattttttaccattttatggaaaatatttaaaatctgtttttacagggttttttttttttttttttttttgtaatctgtgccatgaaatttgaaaaccaccaaaaatcaagggaacttttatatattcaattccttttctggtgtaatgttaaagttgtatagattattaatgcatgcccactgaatataaccctggttttgtgataaaactgcttagattttgttgatgacattagattagtagttgcattaaataactaaattcccattgtgattaattgaaattttgtctttaagcagagagttatttgtgactataagctttgtgcttagagaatgtatgtgtttttatctgtcagtatgggaggatataaactgcatcattagtgaaattattggttgtgtaatcctttgtgaaatataattctaggtatttgatagggtattgagtgtattttgtgtgtgtgtggatgtgtgttttggggtacggggagaggcgatgctattggccatcactaccaaccagggtttcaaaaagtattacctaagtaatttcttttatcactatctcaactgaggaagaaaaggctcaccacaagtggtgtgaaggctttgggtacttagttctaaatttttttatggtaacatatacatagccacatttacagttttaaccattttaaggcatgtaattcagtggggttaggtacattcacaatgttgtgtaatgatcaccgctgtctacttgtaaaactttttcatcaccccaaacagaaactctgtgtgcaattaaagtaatgcatttctcttcttcttaa

SEQ ID NO: 10 - protein, 336 aa, Homo sapiens
MSQAVQTNGTQPLSKTWELSLYELQRTPQEAITDGLEIVVSPRSLHSELMCPICLDMLKNTMTTKECLHRFCADCIITALRSGNKECPTCRKKLVSKRSLRPDPNFDALISKIYPSRDEYEAHQERVLARINKHNNQQALSHSIEEGLKIQAMNRLQRGKKQQIENGSGAEDNGDSSHCSNASTHSNQEAGPSNKRTKTSDDSGLELDNNNAAMAIDPVMDGASEIELVFRPHPTLMEKDDSAQTRYIKTSGNATVDHLSKYLAVRLALEELRSKGESNQMNLDTASEKQYTIYIATASGQFTVLNGSFSLELVSEKYWKVNKPMELYYAPTKEHK

SEQ ID NO: 11 - DNA, 4678 bp, Homo sapiens
agtctcgtccggagactggcagcggcggcggcggcggcggccggagctcgagccccagcggctgagggcgggcgggcgggcgcgggggagggaggggggccggtccgcgacgactccccggacggcgtttctcctccgagcggcgccggtttcggcttggggggggcggggtacagcccatccatgaccatgggcgacaagaagagcccgaccaggccaaaaagacaagcgaaacctgccgcagacgaagggttttgggattgtagcgtctgcaccttcagaaacagtgctgaagcctttaaatgcagcatctgcgatgtgaggaaaggcacctccaccagaaaacctcggatcaattctcagctggtggcacaacaagtggcacaacagtatgccaccccaccaccccctaaaaaggagaagaaggagaaagttgaaaagcaggacaaagagaaacctgagaaagacaaggaaattagtcctagtgttaccaagaaaaataccaacaagaaaaccaaaccaaagtctgacattctgaaagatcctcctagtgaagcaaacagcatacagtctgcaaatgctacaacaaagaccagcgaaacaaatcacacctcaaggccccggctgaaaaacgtggacaggagcactgcacagcagttggcagtaactgtgggcaacgtcaccgtcattatcacagactttaaggaaaagactcgctcctcatcgacatcctcatccacagtgacctccagtgcagggtcagaacagcagaaccagagcagctcggggtcagagagcacagacaagggctcctcccgttcctccacgccaaagggcgacatgtcagcagtcaatgatgaatctttctgaaattgcacatggaattgtgaaaactatgaatcagggtatgaaattcaaaacctccacctgcccatgctgcttgcatccctggagaatcttctgtggacatcgacctcttagtgatgctgccaggataatttctgcttgccatgggcatctggccaccaaggaatttcgcaccctgacgattactcttgacacttttatgtattccattgttttatatgattttcctaacaatcatttataattggatgtgctcctgaatctactttttataaaaaaaaaaaaatctgctgtgcacaattttccatgtacattacaactggttttttgtttttgttttgttgccggtggggagggctgggagggggagggaacttttatttattgtgttcacaaactccatcctttcagcatatccttttaagtttagttctttcttccagttatactatgtactatcagttttgatataactatatatatataaatataaaattatatataaagggttatttgaaaccaatccatggcaacgctggtgcttgatacactgtgaagtgaatacaacattgaacagttacagatctgggacagtcccttctatgaaagtgctgaaatttaattaaaatcagtcttatatgaagtatgttccaatccatgtgggaacttgactctctcatctgtctaaagagtactggacgatataaaaatatatattttttaaacaatgtgatctcaaatttaaagactgctccagatagcctgcatttgcaatggaataactgacaaatcacaagtggtttagttgggcagggctttgatcattcaaaagtaactaaagtagctccagaatgccaagtattcgtgtaaattacggttacatgttatcatttgctgttcttacataagcactcatgaaaatatggtattctgtaacttgaattccatccattttccagacctctactcatgtctgaggtaaatctagaaattgtcttagttttaggattgaaacagtctataaactgtatttttggtccatccaggaagctagtcccttgtttctcctttctacatgacattgcagtggtggtttctgtaattaaaatttgtttgcctcatgtccctttgtctgataaaccttcactctaccgattcagttgtgagcattctttttttccttctcaaaacctactatgatttgttttactgaacaaaggttatcaaccacacatccagtcctgacatggagcttttcagtgtttggagacatttctcaatcccctgctgtggtaggaactccagtggtgaacggcttgcgcgcctgcagccagagttgcagggaaagctcgtacttactgcgagcagcatgtaatcttttttcttcctggacataaagatagcttgagtaaactgttctatttcattctcttcactctttttactgtcttgcaaaaaaaaaaaaaaaaaaaataatcaaagaccactaataagattccacctctccttattaaaataattttttaaaattttgttttgcttttgtttggatgtggggtctctcttctatttgacttttacatttagatacagagtttgtagtacttcagagacatttcaagcatgagaatttgaggttacctctctttatttgacctttagggactcacgggagggcagcctgatttgtaatgaagcaccacattttggtgttaaaaacctggtttgcttaataatagcagtaatttctgtctgtggaggcaacaaataaaaaaattaacagcttgaattgagtagccaacaggaaaggttcctttcacatttacattaaaactattctgtagtcactaatgtaccataatttaaattcttttctcaaaggtatagattataaagcagtgccatttgttgctgtggtcctattctcaaatgcatggacaatgttcccccctttttaaaataatgcttgtgtctgggatgcaagctttgcttatctttttaaatacatttttaaagtatttattaatgaaccaaaggaaatcagatgctttctataagcatcagaatatataatacatagtgatttgactatgaattttaaatccacattttaatattggtgggatattgcaaagacattccttctaaagttttaatattccttttattaagggtctcagggagggtaaattagtcagccatatttattttccagaggtttaagaaattgctgtttttaactttttgaaaaaacttaaatgccaccaaactcatgtaggttgcactgcttattgaaccaataactgttggtatgcactttgttcagacacactgtgtactttttcaaaaactagtttcatgtaaagtgattggaccccatagattagtggaaaaagctgattaaccagctactcataggctgctaattcattcatgccaatgttttggtttttcagttttgcctccgtgataaattaaagaatggggaggggtgaaggaaggggaagaagattgctttagaacaagtggcatgaaattaccatctttgtagaaaccgcagctaacagtgggagttatctaagcaatcagatgttacagggccagccctttagctgctgtggtgtattctgttgggtagtgaggtagtaggtactttatagacttttaattttggaaattgatgacatccctcaggcatgtattctggaaatggaattcctgtaacttcctgtgtctgcagtatgccctacaattagtaggcagcgtgtaaaaacactagtgtagattataaagatatacattaaaagaggaccagaaatacttggtattcagtggcacagaaagcaggttaaacaaacaaaaagcacagtgttacgcttgcaagtttccatttgttttaataccacgcaatctttcacactcgtgcgtgtgcgcgcacacagagcttacctgacttgctctgcttgagtcatgcagttacaaaaaaaaagacatcttgacacccacacaatattctaatcaaaacctttcagtttcaatctggatatttaaaaacattggcagaagcttctgtgagtttagttccactaagatgtttcacctgccttatcaagaccattctcagtctacttttttaagctaccgtatcttaaattattgaaaatttattaattgctgaatatataataacctttgcttgtatgtaaccgaaaatggtttaagagccaacatttagagtatgacaatggagctgaacagtttttaatgcgcaagcagttctgttcttgtgtatgacttgtaaccttaatttactgtgtaaagatggttacattatttccttagctttgtttgttggagacaaatagagaatgcttgttaagtatgtcaaaacaatcttatcttgtgaatttttgttaatgtattatacgagctatatttttcatttgcccagaaagacagcttgtataacgcttttggaagtttctgctctgtaatgtctttagagctgacagtctgttaggtttgtttttttcttcatgctaaagtgtcagttggtggttttgtgaactggtcaaaaattcacaggtcttaaatgttttgggggaaatttatattggacactgctctttgtctagcaaataaaagatgttaatatattcctgttactggcatgtgcacgactatgttattagaagccactttatcattttcctgctttaaatagaaatgtctatttatgaattctgcttgtagttttttcacaaataaaatagtaaaatttccattggaaatcttaaaaaaaaaaaaaaaa

SEQ ID NO: 12 - protein, 228 aa, Homo sapiens
MTMGDKKSPTRPKRQAKPAADEGFWDCSVCTFRNSAEAFKCSICDVRKGTSTRKPRINSQLVAQQVAQQYATPPPPKKEKKEKVEKQDKEKPEKDKEISPSVTKKNTNKKTKPKSDILKDPPSEANSIQSANATTKTSETNHTSRPRLKNVDRSTAQQLAVTVGNVTVIITDFKEKTRSSSTSSSTVTSSAGSEQQNQSSSGSESTDKGSSRSSTPKGDMSAVNDESF

SEQ ID NO: 13 - DNA, 907 bp, Homo sapiens
agtggccggctgggatcagcctttaagatggcgtctcctcaggggggccagattgcgatcgcgatgaggcttcggaaccagctccagtcagtgtacaagatggacccgctacggaacgaggaggaggttcgagtgaagatcaaagacttgaatgaacacattgtttgctgcctatgcgccggctacttcgtggatgccaccaccatcacagagtgtcttcatactttctgcaagagttgtattgtgaagtacctccaaactagcaagtactgccccatgtgcaacattaagatccacgagacacagccactgctcaacctcaaactggaccgggtcatgcaggacatcgtgtataagctggtgcctggcttgcaagacagtgaagagaaacggattcgggaattctaccagtcccgaggtttggaccgggtcacccagcccactggggaagagccagcactgagcaacctcggcctccccttcagcagctttgaccactctaaagcccactactatcgctatgatgagcagttgaacctgtgcctggagcggctgagttctggcaaagacaagaataaaagcgtcctgcagaacaagtatgtccgatgttctgttagagctgaggtacgccatctccggagggtcctgtgtcaccgcttgatgctaaaccctcagcatgtgcagctcctttttgacaatgaagttctccctgatcacatgacaatgaagcagatatggctctcccgctggttcggcaagccatcccctttgcttttacaatacagtgtgaaagagaagaggaggtaggggccaagcccccaccccatcccactccccttccctccccagatatttatgtgaaatgaactgcagctttattttttgaaataaaaacttttaaaaagca

SEQ ID NO: 14 - protein, 259 aa, Homo sapiens
MASPQGGQIAIAMRLRNQLQSVYKMDPLRNEEEVRVKIKDLNEHIVCCLCAGYFVDATTITECLHTFCKSCIVKYLQTSKYCPMCNIKIHETQPLLNLKLDRVMQDIVYKLVPGLQDSEEKRIREFYQSRGLDRVTQPTGEEPALSNLGLPFSSFDHSKAHYYRYDEQLNLCLERLSSGKDKNKSVLQNKYVRCSVRAEVRHLRRVLCHRLMLNPQHVQLLFDNEVLPDHMTMKQIWLSRWFGKPSPLLLQYSVKEKRR

SEQ ID NO: 15 - DNA, 9386 bp, Homo sapiens
gctgtagtggcttcgtcttcggtttttctcttccttcgctaacgcctcccggctctcgtcagcctcccgccggccgtctccttaacaccgaacaccatgccttcaattaagttgcagagttctgatggagagatatttgaagttgatgtggaaattgccaaacaatctgtgactattaagaccatgttggaagatttgggaatggatgatgaaggagatgatgacccagttcctctaccaaatgtgaatgcagcaatattaaaaaaggtcattcagtggtgcacccaccacaaggatgaccctcctcctcctgaagatgatgagaacaaagaaaagcgaacagatgatatccctgtttgggaccaagaattcctgaaagttgaccaaggaacactttttgaactcattctggctgcaaactacttagacatcaaaggtttgcttgatgttacatgcaagactgttgccaatatgatcaaggggaaaactcctgaggagattcgcaagaccttcaatatcaaaaatgactttactgaagaggaggaagcccaggtacgcaaagagaaccagtggtgtgaagagaagtgaaatgttgtgcctgacactgtaacactgtaaggattgttccaaatactagttgcactgctctgtttataattgttaatattagacaaacagtagacaaatgcagcagcaagtcaattgtattagcagaatattgtcctcattgcatgtgtagtttgagcacagatcccaaaccttacggccaagtttcttctagtatgatggaaagtttcttttttctttgctctgaataaaactgaactgtgggttctctataagtggcattttgggctttccctcttttttgtaaagcaatgtctgcctagtttattgtccagttaactttagtgaccttttaaaagttggcattgtaaataaaacaacttgcaaaaaagttttctggaatagaattaacaaaatattatctttattcatgagttggaaactggaaaaaggcttcttgaagtaaatgttctgagtggagctactaggatgtcttccagcctcctgcagtcaaggagtaccactgtattgattagcctgtatgtagcagggctcccttcattgcatctgaggacttgttttctttttctttatttttaatcctcttagttttaaatatattgcctagagactcagttactacccagtttgtggttttttgggagaaatgtaactggacagttagcttttcaattaaaaagacacttaacccatgtgggatgtcatctttttataattagtgttcccatgtggagaaaattattcacactacttgcatgtaaagaataatttaacttttaacattaaaatatgtggtaaaacccagaaagcatccatcatgaatgcaagatactttcaataaaaagtaagttatatagtaggtagttaagtttgcttttgtggacttaaatgtgtctcttcacttaaatgggttgaatgtgtatatatttgttcagcttgaaaagacttagtttatatcctagctcactggaggctgctgacataaccataacttctgtcccttctaattgtcatttatatgcctaactggagctagtactttaattcttaacacaaaattactctgccattgtttccagcttccctcctacaatagaatgaagtttttttgatggcttgagatggctcacaaattttgatttttttttcttccttgtgctccctttttttctccttgcttttccagttaacatctatattcacatgtaatcttgttttctcttcacattcactgagttgttcaggctcagatcatcccttgacagtagtttgccttcatctcacctttcatttgtcccaaattcaccttatttaataaagtcccatatgttgtctcacttaattccaatttgttgtctgtaccagagataggaaactataatgtgtgggtcaaatcaggcccatagcctgtttttgtagtctttgagctaaaaatggtttttacgttgtaaagagttatttaaaattctctctctctctctctctctctctcaggagataagcttgttttctgtttgtcctggttttagtggggaaggtagcggtgtatagtcctagctgaattgtctcatctaccaattcctgctattataagatcaacttctgcaaaaaccttatccacctcaagtatccttagcagctgggcatggtggcttatgcctgtaatctcaacattttgggaggacaagacaggaggatcgtttgagcccaggagttcaagaccagcctgggcaacacagaaagtccctgtctctccaaaaaaaaaaaaaaaaattagcagtcatatggtgcatgcctataatcccaactacttgagaggctgaggtgggaagatggcttgagcctgagaggtcgaggctgcagtgagctggcattgcaccactgcactccagtctaggcaatagagcgagaccttgtctcaaaaacaaaccagaagtatccttagcagtattatgacaaacaaatcttcactgaaggtccaggattactgccatatgccatgtttaacttgtaaaggaagtatcaccttctaagaaactggagcttgttattaccacttgatctgtataatactcagcagtagttagaattatatgagtaaataacgtcactatccgtatttaaaggataatgagatctttcagaaagattggatggacagacaatatagtagtaatttaaatttttgtttcctgactttctgtagtccctaaacaaacaacaaaaaatcctagtaatcttaaacttttacattaatagagatccaagagaaaataagccatttttcaccattgtggacccaaataaatcatagacatggtattaagaagcccttttcagtctggttgcagtattattttcctacctttctttttcctgttccctgctgtatgcccactattcctttaatatatctactagaacttttctaggctttcacctggagtgagtggtcctttctcattatcacagtagccaaacctcagtcttcaaaactccactcatttctggctactctcttacttcccaactattcttctaaactaatattgtggcattaagtcataccttgaggctggagttttagtgtcttcatggccttgggcaagttggaaataggtactacggttttatagatctagtgcagtctgctttatattagttccagaacttcattggaagtcacttgacagcaggatttaacttgtatttagcagcactactccatgaatttcagtataagtaacagaagtgaaaagtccttgtgaaatgcaggtacagggcataatgaaaacaagaagagtagttttaatgcatgagaggggagagcttgataatagctgataatagccttggtttggagagtggttactgatgaattatgaagactttctctaattactgttatagtagtaaaggaaagaaaacccttgttgataaagtaaacttagggattaattaggaaatgccttttatttcaccaggaaagagaatgagaggaagaaggtatttggcagatttgggtgattgaaggatgtttgtcggtttcctctgtaaacaacatgctgctttctgcagtgtgctgcttttaatgatgaagtcctttcaaggactccatggcagtcctttggcttctcaccttttcattgattatgtcacttgtgatctaaaagtaaaccaaaaccttcctgaatgttagcttattattttctccttaacattgtcataggctaaagatgtgccccctcaaattctaataatttttttttttttttttttttttttgagacagagtctcactctgtcgcccagactggaatgcagtggtgcgatctcagctcactgcaacctccgcctcctgggttcaagtgattctcctgcctcagcctcccgagtagctgggattacaggcacatgccaccatgcctggctaatttttttgtgtttttattagagatggagttttaccatattggccaggctggtcttgaactcctgaccttgtgatccgcccgccttagcctccgaaagtgctgaggttacacgtgtgacccaccgcacacggccctaataattcttaagttgtagaagcaagttactcttttgaagtgccatgtgttagcttgccttaatctattcttggtatacaaaatagatactgctttacagttcttatattttctcagagattcacaaaatcatcctgtagctccctgtgtgggattatagctgtgactttttactctacaactgtcagaatacaccagctcctggaattaagcaagaactatattgtgacttggttggacaggtaactgcagccttgtaacatgacatcatagtaatgctgagcttatcttaaaactagctggggagggaggatagtaacagtgagtcactgtgggcatctcacttagggcaggcagatattccaaatcacattgatttttcagtttacgtaaaaattgaccaatcctatgggctgtgttctgatcccaacataaattttgtttaagttaatttgcctaggctctcaataaggcagtttgggaatataaggtgattttccaccagaaaagagaaaattgagagagcagcagacctcttgctctcagcaccctttacaagcttaacttttgcttgccaccattagcttttgaagattttttttaactgctccttgcagagcaggactaccccataggcagtgtgcccagagtagcccgaagagctttttgattcttcttttaagagacaaggtctcacctctgccccccaggctggagtgcagtgatgtgatcacagctcattgcagcctcgaactcttgggctccaacagtcctcctgcctcagcctcctgaatagctaggactacatgtgtgtgctaccatgccttgctagtttttgtttttttaaattgatttttatagagatgaggtctcctcatgttgcccaggctgttttcgaattcctgggctcaagatcctcctgcctcaacctcccaaagtgctggtattacagatgtgagccacagcacccaccaatttttccttttctaaagctcagtgtacattctggtaggaaggaatcaaccaagtaaatgttctccctacaaagttgtttgaggatttgaaaagttaatcttcaattatctggatttgaaaccagtctataactttttaagctaggaagaagtcaagaataagaagattgcaagcaaagggaaagcttacaaaatggctgaatccttaaagtgtagctcctggcttctccttggtaggaagcattagacatggtttccctctagggagctgggagcttttggtgtagttcaggagagaaggcataaatactgctgggagggagtggataactctaataaaaatgttcctgggctgggcacggtggctcacgcctgtaatcccaacattttcagaggctgaggtgtgcagatcacgaggtcaagagatcgagaccatcctggccaacatggtgaaacctcatctctactaaaaatacaaaaattagctgggcgtggtggcacatgcctgtagtcccagcaactcgggaggctgaggcaggagaaccgcttgaacccgggaggcggaagttgcagtgagccgacatcacgccactgactgcagtccagcctggcgacagagcgagactccgtttcaaaaaaaaaaagtttctggacagaacagattaatgagccaatggggggctacagagctgaaaggtgctacgaaaggttttgtaaagtgaaactaatttggtttcaagaatcaagttggatttgtttgggggtggacagatgccaaagtgaggaaaaagcagtgcacagtcaggaactaacagcagaaggttgggtgttggtacatttccctgagaatatagactaaagagaaattagggcaggattagtcacagtactttgctgacactcagtagtattttatcagcatttattcatcagacccacattaggcagggcaatttttgagtgagctaaattctaatttttttaattttaatttttatttatttattttttggctgttcaactaaaccagtcttttgaatgttgggggttttacagttagagtcctgatgactttttcttgtttactgttgttaaaaatcactacctccagtggtcacagagaatgagtaagagaactggctgtgcatatcaagatgaagtatggtgtaagaaatatttcagagcatgtcctttggcccaagattgagccatttggacacctaagcaggattggctgaatgttatcctttctgctgaggcatcagtggagatgatcattagtggtggccgaaaagtgttgtcgaaggtgctcttgcttgacacacttccaatgcgggacatagtttgagtgatagatggctaattcattgataatgtacttgttgccttgcagtcttgtgtactgctggctacctctggtctgtgtgccagtattgagcagcattctatgggaggaatgtagttgctcataacagtgtctgcttccacatctgtagttcttgcgaccttctgatatctgtcaactttaaattttctggaagtatttaagtatattcagaccttctgatatctcttaacttcaaattttcaacctgctatatagcagcctcttattgcacactgaggatcagcttatttccttaaagtgctctcactttgaatacctcccacctatctggtgaaaattcttatttgtcctttctttcctgaatgtaccatttcacaacctctaactgccttcttcagttgctttgcagatttttctgttccaggaagcttacggaaaggggtgggaaaatagagaagctgtactctatcctcaagtactcaagcagggtttccagagtggctttgtaaaaatttttatttatttatttatttatttttggtgagtggaacctggcttttcctcttccctccccaagccccaccccgcttcctgacattgcacatgcctgaaagaactaggcttcagataaaagaatgagaacacttgaatgaaaaaggaagacttggtccagagcccctggtgaatatgaaagaagtattggcctggactatctcccgcataattcacagctgctatcttgtcttcagaagtgaaacatcccttaccaatagtgacctaacttgtgaggactgcttgtgatagtatagttcccacccccaggacataagttttccctttccagttaacatgcctaatcccatgctatctttaagagcttgaaaatccttcctgacctgagctaggcttttattctaagggactgtgtatgtgaacatccccaccatctatgtttcctgttttcaaactcggtgccgttttgagtattttcagatgctcactgccagtaatggtttgttagtagctccaaactattagagaagagctcaggagtcagaatttgaaagatgcccatagggtgatttgagatttggaacaaggttatcacaataagttgggattgggggagttgacacaaccagggtctgcttgggtgggcagctgtttcagaaacccttggtgaactcttaaagccctgaagagcttaaaaaatgctctgaagagatctgagagtcgcagttaaggactctcaaagaacctcaaagaaaagccaacttagcctttttatacaattaactagatttgggtcttaaattctactttgacatataggcctaagaggtagtttaacatggccttgttacccttaccccttctgttctgtgctgttttctgagaaacggatgcaggaactgattccctttcttcctctctggaacaggatataaagaacccttccgcttccctttacttgtggccttcctggccaggcttatgtatactgaacaaagctagggtgcagtgcttctgagctagctgttaggggtccacagccagaaggaatgggagaccacaggagccattcaaaagataattacatgtcagaagagagtccatccatggggttctgttaaagtagacaccttcacgttgggagacataggctttgtctgcttgctgggctgaaccctcaccatatccacagagaacaaaatggggagcagctgtaccaggggagggatgaacgtctttcacccaagttgccagtctgctagttgtctggaaatgttcaagggaggctaaggtatcttgtcttatgatgtctcaggaaacagggttggttctcagctggcagtgaagggggactggtgtggtattagcagttgaacttgaggttgcaatctcaagtcaccctctggcctcgcaggaatgcaccagggtgatttgggaggatctgagtttcctttatgaagtcagattaaaaaaaaaaaaagctgatctgatttaaacagagttgccaaagacttccccgccaatacacaaagactgcattgttcaggtaccctgtgtactctaagtgagacaccaaatcagatgcagcaacctggtcttgcataagccctggttagaatgtaggttttttaaacaactaggccagtagccagaacagaattctttaaaatgggacagatgtccagagaatggctagtgtcctctagcactctctcagaagcaaaagcaagtctggggttacaacttccagtggcctactttaaatagacgctgttctggaaaatgactttatactttaaacacaaaatccaaaaaatagaactcagtggagtgacaatgacaatgcatgatggtggcccagtggctgtcccatgtgaaatacttcttgcctgacatgtatcatctacttgttgactgacctattgaggtgccttcatgacaccttttacattcatgataggtctcaggaagacagcagtgtacttggtggaaactcatgtaaaaagtgggttgtagggagctaaacagtgagtacacatggtatatggatacggaatggaataatagacattggagacttcaaaaggtgggagatgaaagggggatgaggtatgaaatcctacctgttgagtacaatgtacactacttacgtgcacagtacactgtttgggtgagaggcacactaaaagcccggacctcaccgctacccagtatgttcatgtaacacagctgcacttgtaccccctaaatgtatacaaataatctaaaaatagcccggacctcaccgctacacaatatattcatgtaacacagctgcatttgtaccccctaaatgtatacaaataatctaaaaataaagtggatatggtggctgtcgtgtccaccaacagcgta

SEQ ID NO: 16 - protein, 163 aa, Homo sapiens
MPSIKLQSSDGEIFEVDVEIAKQSVTIKTMLEDLGMDDEGDDDPVPLPNVNAAILKKVIQWCTHHKDDPPPPEDDENKEKRTDDIPVWDQEFLKVDQGTLFELILAANYLDIKGLLDVTCKTVANMIKGKTPEEIRKTFNIKNDFTEEEEAQVRKENQWCEEK

SEQ ID NO: 17 - DNA, 3522 bp, Homo sapiens
gagtgcgggccgcgctctcggcggcgcgcatgtgcgtgtgtgctggctgccgggctgccccgagccggcggggagccggtccgctccaggtggcgggcggctggagcgaggtgaggctgcgggtggccagggcacgggcgcgggtcccgcggtgcgggctggctgcaggctgccttctgggcacggcgcgcccccgcccggccccgccgggccctgggagctgcgctccgggcggcgctggcaaagtttgctttgaactcgctgcccacagtcgggtccgcgcgctgcgattggcttcccctaccactctgacccggggcccggcttcccgggacgcgaggactgggcgcaggctgcaagctggtggggttggggaggaacgagagcccggcagccgactgtgccgagggacccggggacacctccttcgcccggccggcacccggtcagcacgtccccccttccctcccgcagggagcggacatggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcggagaatgaagaaattgatgttgtgacagtagagaagaggcagtctctgggtattcggaagccggtcaccatcacggtgcgagcagaccccctggatccctgcatgaagcatttccacatctccatccatcagcaacagcacaactatgctgcccgttttcctccagaaagctgctcccaagaagaggcttcagagaggggtccccaagaagaggttctggagagagatgctgcaggggaaaaggaagatgaggaggatgaagagattgtgagtcccccacctgtagaaagtgaggctgcccagtcctgccaccccaaacctgtcagttctgatactgaggatgtgaccaagaggaagaatcacaacttcctggagcgcaagaggcggaatgacctgcgttcgcgattcttggcgctgagggaccaggtgcccaccctggccagctgctccaaggcccccaaagtagtgatcctaagcaaggccttggaatacttgcaagccctggtgggggctgagaagaggatggctacagagaaaagacagctccgatgccggcagcagcagttgcagaaaagaattgcatacctcactggctactaactgaccaaaaagcctgacagttctgtcttacgaagacacaagtttattttttaacctccctctcccctttagtaatttgcacattttggttatggtgggacagtctggacagtagatcccagaatgcattgcagccggtgcacacacaataaaggcttgcattcttggaaaccttgaaacccagctctccctcttccctgactcatgggagtgctgtatgttctctggcgcctttggcttcccagcaggcagctgactgaggagccttggggtctgcctagctcactagctctgaagaaaaggctgacagatgctatgcaacaggtggtggatgttgtcaggggctccagcctgcatgaaatctcacactctgcatgagctttaggctaggaaaggatgctcccaactggtgtctctggggtgatgcaaggacagctgggcctggatgctctccctgaggctcctttttccagaagacacacgagctgtcttgggtgaagacaagcttgcagacttgatcaacattgaccattacctcactgtcagacactttacagtagccaaggagttggaaacctttatatattatgatgttagctgacccccttcctcccactcccaatgctgcgaccctgggaacacttaaaaagcttggcctctagattctttgtctcagagccctctgggctctctcctctgagggagggacctttctttcctcacaagggacttttttgttccattatgccttgttatgcaatgggctctacagcaccctttcccacaggtcagaaatatttccccaagacacagggaaatcggtcctagcctggggcctggggatagcttggagtcctggcccatgaacttgatccctgcccaggtgttttccgaggggcacttgaggcccagtcttttctcaaggcaggtgtaagacacctcagagggagaactgtactgctgcctctttcccacctgcctcatctcaatccttgagcggcaagtttgaagttcttctggaaccatgcaaatctgtcctcctcatgcaattccaaggagcttgctggctctgcagccacccttgggccccttccagcctgccatgaatcagatatctttcccagaatctgggcgtttctgaagttttggggagagctgttgggactcatccagtgctccagaaggtggacttgcttctggtgggttttaaaggagcctccaggagatatgcttagccaaccatgatggattttaccccagctggactcggcagctccaagtggaatccacgtgcagcttctagtctgggaaagtcacccaacctagcagttgtcatgtgggtaacctcaggcacctctaagcctgtcctggaagaaggaccagcagcccctccagaactctgcccaggacagcaggtgcctgctggctctgggtttggaagttggggtgggtagggggtggtaagtactatatatggctctggaaaaccagctgctacttccaaatctattgtccataatggtttctttctgaggttgcttcttggcctcagaggaccccaggggatgtttggaaatagcctctctacccttctggagcatggtttacaaaagccagctgacttctggaattgtctatggaggacagtttgggtgtaggttactgatgtctcaactgaatagcttgtgttttataagctgctgttggctattatgctgggggagtcttttttttttatattgtatttttgtatgccttttgcaaagtggtgttaactgtttttgtacaaggaaaaaaactcttggggcaatttcctgttgcaagggtctgatttattttgaaaggcaagttcacctgaaattttgtatttagttgtgattactgattgcctgattttaaaatgttgccttctgggacatcttctaataaaagatttctcaaaca

SEQ ID NO: 18 - protein, 364 aa, Homo sapiens
MDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEEVLERDAAGEKEDEEDEEIVSPPPVESEAAQSCHPKPVSSDTEDVTKRKNHNFLERKRRNDLRSRFLALRDQVPTLASCSKAPKVVILSKALEYLQALVGAEKRMATEKRQLRCRQQQLQKRIAYLTGY

SEQ ID NO: 19 - DNA, 6910 bp, Homo sapiens
agacggagcctgggctcccagcggcaaggtgaggcagagctgcgctcctcgctgaacgcgggccgagctcggcggctgcgggggagacgcgcaggagcccagaccgcgaccgagagcgggagctaggcgggcggcggcggcggagggggagcccgcgagccgccgggcggagagcccaagccgcgctgtcgccgcgcagggacgacttggccaacactcacacacactcacacacacccagcccgagcgggcgctcgcggcgaaccgtcaacatggcgctggggctcctgcccgagcgcgggcggcggcggcagcgcgggagctgctgagctcggccaagcccagtccagctgcgggagcccggaggatcgcacggggctgtcgccacctgcccggaggccccgagcccgccccgccccgcccccacccggcccagagcccacccctcggcggggccgaccccgagggcagccggctgccagcagacggcgagggagtcgagtgagcgcggcgccgcgagcgggctgcgggcagccggggaccgcaaactttgctgctcgccgcgcttctccggcccggctccttctccgctcgttaacgtcgccaaccccccccacccctcatatctctctccacccacccaaccgccccccgctccttctcgccgcctcgagtccgcttgggggaaaacttcaaagagccggatcgcaggctccctgcctactcccccaccggggatttcagactagacgcttgaagcaaagctgccatcccagaagacgacatgctctcagcaacccccctgtatgggaacgttcacagctggatgaacagcgagagggtccgcatgtgtggggcgagcgaagacaggaaaatccttgtaaatgatggtgacgcttcaaaagccagactggaactgagggaagagaatcccttgaaccacaacgtggtggatgcgagcacggcccataggatcgatggcctggcagcactgagcatggaccgcactggcctgatccgggaagggctgcgggtcccgggaaacatcgtctattctagcttgtgtggactgggctcagagaaaggtcgggaggctgccacaagcactctaggtggccttgggttttcttcggaaagaaatccagagatgcagttcaaaccgaatacacccgagacagtggaggcttctgccgtctctggaaaacccccaaatggcttcagtgctatatacaaaacaccgcctggaatacaaaaaagtgctgtagccacagcagaagcgctgggcttggacaggcctgccagcgacaaacagagccctctcaacatcaatggtgctagttatctgcggctgccctgggtcaatccttacatggagggtgccacgccagccatctaccctttcctcgactcgccaaataagtattcactgaacatgtacaaggccttgctacctcagcagtcctacagcttggcccagccgctgtattctccagtctgcaccaatggggagcgctttctctacctgccgccacctcactacgtcggtccccacatcccatcgtccttggcatcacccatgaggctctcgacaccttcggcctccccagccatcccgcctctcgtccattgcgcagacaaaagcctcccgtggaagatgggcgtcagccctgggaatcctgttgattcccacgcctatcctcacatccagaacagtaagcagcccagggttccctctgccaaggcggtcaccagtggcctgccgggggacacagctctcctgttgcccccctcgcctcggccgtcaccccgagtccaccttcccacccagcctgctgcagacacctactcggagttccacaagcactatgccaggatctccacctctccttcagttgccctgtcaaagccatacatgacagttagcagcgagttccccgcggccaggctctccaatggcaagtatcccaaggctccggaagggggcgaaggtgcccagccagtgcccgggcatgcccggaagacagcggttcaagacagaaaagatggcagctcacctcctctgttggagaagcagaccgttaccaaagacgtcacagataagccactagacttgtcttctaaagtggtggatgtagatgcttccaaagctgaccacatgaaaaagatggctcccacggtcctggttcacagcagggctggaagtggcttagtgctctccggaagtgagattccgaaagaaacactatctcctccaggaaatggttgtgctatctatagatctgaaatcatcagcactgctccctcatcctgggtggtgcccgggccaagtcctaacgaagagaacaatggcaaaagcatgtcgctgaaaaacaaggcattggactgggcgataccacagcagcggagttcatcatgcccgcgcatgggcggcaccgatgctgtcatcactaacgtttcagggtcagtgtcgagtgcaggccgcccagcctccgcatcacccgcccccaatgccaatgcagatggcaccaaaaccagcaggagctctgtagaaaccacaccatccgttattcagcacgtgggccagcccccggccactcctgccaagcacagtagcagcaccagcagcaagggcgccaaagccagcaacccagaaccgagtttcaaagcaaacgagaacggccttccaccaagctctatatttctgtctccaaatgaggcattcaggtccccaccaattccctaccccaggagttacctcccttacccagcccctgagggcattgctgtaagtcccctctccttacatggcaaaggacctgtctaccctcacccagttttgttacccaatggcagtctgtttcctgggcaccttgccccaaagcctgggctgccctatgggcttcccaccggccgtccagagtttgtgacctaccaagatgccctggggttgggcatggtgcatcccatgttgataccacacacgcccatagagattactaaagaggagaaaccagagaggagatcccggtcccatgagagagcccgttacgaggacccaaccctccggaatcggttttccgagattttggaaactagcagcaccaagttacatccagatgtccccaccgacaagaacctaaagccgaaccccaactggaatcaagggaagactgttgtcaaaagcgacaagcttgtctacgtagaccttctccgagaagaaccagatgctaaaactgacacaaacgtgtccaaacccagctttgcagcagagagtgttggccagagcgctgagccccccaagccctcagttgagccggccctgcagcagcaccgtgatttcatcgccctgagagaggagttggggcgcatcagtgacttccacgaaacttatactttcaaacagccagtcttcaccgtaagcaaggacagtgttctggcaggtaccaacaaagagaacctagggttgccagtctcgactccattcctggagccacctctggggagcgatggccctgctgtaacttttggtaaaacccaagaggatcccaaaccattttgtgtgggcagtgccccaccaagtgtggatgtgacccccacctataccaaagatggagctgatgaggctgaatcaaatgatggcaaagttctgaaaccgaagccatctaagctggcaaagagaatcgccaactcagcgggttacgtgggtgaccgattcaaatgtgtcactaccgaactgtatgcagattccagtcagctcagccgggagcaacgggcattgcagatggaaggattacaagaggacagtattttatgtctacccgctgcttactgtgagcgtgcaatgatgcgcttctcagagttggagatgaaagaaagagaaggtggccacccagcaaccaaagactccgagatgtgcaaattcagcccagccgactgggaaaggttgaaaggaaatcaggacaaaaagccaaagtcggtcaccctggaggaggccattgcagaacagaacgaaagtgagagatgcgagtatagtgttggaaacaagcaccgtgatccctttgaagccccagaggacaaagatcttcctgtggagaagtactttgtggagaggcagcctgtgagcgagcctcccgcagaccaggtggcctcggacatgcctcacagccccaccctccgggtggacaggaaacgcaaagtctcaggtgacagcagccacactgagaccactgcggaggaggtgccagaggaccctctgctgaaagccaaacgccgacgagtctctaaagatgactggcctgagagggaaatgacaaacagttcctctaaccacttagaagacccacattatagtgagctgaccaacctgaaggtgtgcattgaattaacagggctccatcctaaaaaacaacgccacttgctgcaccttagagaacgatgggagcagcaggtgtcggcagcagatggcaaacctggccggcaaagcaggaaggaagtgacccaggccactcagcctgaggccattcctcaggggactaacatcactgaagagaaacctggcaggaaaagggcagaggccaaaggcaacagaagctggtcggaagagtctcttaaacccagtgacaatgaacaaggcttgcctgtgttctccggctctccgcccatgaagagtctttcatccaccagtgcaggcggcaaaaagcaggctcagccaagctgcgcaccagcctccaggccgcctgccaaacagcagaaaattaaagaaaaccagaagacagatgtgctgtgtgcagacgaagaagaggattgccaggctgcctccctgctgcagaaatacaccgacaacagcgagaagccatccgggaagagactgtgcaaaaccaaacacttgatccctcaggagtccaggcggggattgccactgacaggggaatactacgtggagaatgccgatggcaaggtgactgtccggagattcagaaagcggccggagcccagttcggactatgatctgtcaccagccaagcaggagccaaagcccttcgaccgcttgcagcaactgctaccagcctcccagtccacacagctgccatgctcaagttcccctcaggagaccacccagtctcgccctatgccgccggaagcacggagacttattgtcaataagaacgctggcgagacccttctgcagcgggcagccaggcttggctatgaggaagtggtcctgtactgcttagagaacaagatttgtgatgtaaatcatcgggacaacgcaggttactgcgccctgcatgaagcttgtgctaggggctggctcaacattgtgcgacacctccttgaatatggcgctgatgtcaactgtagtgcccaggatggaaccaggcctctgcacgatgctgttgagaacgatcacttggaaattgtccgactacttctctcttatggtgctgaccccaccttggctacgtactcaggtagaaccatcatgaaaatgacccacagtgaacttatggaaaagttcttaacagattatttaaatgacctccagggtcgcaatgatgatgacgccagtggcacttgggacttctatggcagctctgtttgtgaaccagatgatgaaagtggctatgatgttttagccaaccccccaggaccagaagaccaggatgatgatgacgatgcctatagcgatgtgtttgaatttgaattttcagagacccccctcttaccgtgttataacatccaagtatctgtggctcaggggccacgaaactggctactgctttcggatgtccttaagaaattgaaaatgtcctcccgcatatttcgctgcaattttccaaacgtggaaattgtcaccattgcagaggcagaattttatcggcaggtttctgcaagtctcttgttctcttgctccaaagacctggaagccttcaaccctgaaagtaaggagctgttagatctggtggaattcacgaacgaaattcagactctgctgggctcctctgtagagtggctccaccccagtgatctggcctcagacaactactggtgagcaagctggacccaccatgtacagtgtgttatagtgttaatccttgtgcatatgtgtcataatacaactatttctgtaaagaaaggacactattacatatgaaaatatctcttctttatataagagaaattactccagtcagaaggacttagaaacatgtttttttccttttaaacttttaagtcagtttttatgaagttgttataatgtttctttacttttcaatgcacacatgctttgggatacgtttgtttttacttggaacatttgtttcttttcttttttaaggagaaaaaaaaatgagtaaaaggagctccacactttgacttaatttcatacaaagctctgatgacaggccatgactgtagagtggtcagaactgtgtggttggtttgagggagcgaattcggggaaggcacttggtgatataactttgttttgtttacagagtacctgctcgggccaggtaaatgctattggatgtaatccagtagtgtgtaatataaattcaaaccatatccacacacaacaactaattgtatgaaacttttatatcctaatttaaaagctgtgaaattagttttcacgcatcaaaccggattgtttatatgtttaaacattttatgctcttatttaaagaagactttgagctatttttttctgtaccctgtaaaatattgaaaactaacataatatgttgaggttgcttggaaatgtacataaaactaaaattttctgaatcgtgtgtttatgtttgaaatctgtgttttaactttgtaagtaaattctctgcctttgtatttatattttacaaaaattttcttaaaaggcaataaaactgttgaggaaaggagaaaa

SEQ ID NO: 20 - protein, 1755 aa, Homo sapiens
MLSATPLYGNVHSWMNSERVRMCGASEDRKILVNDGDASKARLELREENPLNHNVVDASTAHRIDGLAALSMDRTGLIREGLRVPGNIVYSSLCGLGSEKGREAATSTLGGLGFSSERNPEMQFKPNTPETVEASAVSGKPPNGFSAIYKTPPGIQKSAVATAEALGLDRPASDKQSPLNINGASYLRLPWVNPYMEGATPAIYPFLDSPNKYSLNMYKALLPQQSYSLAQPLYSPVCTNGERFLYLPPPHYVGPHIPSSLASPMRLSTPSASPAIPPLVHCADKSLPWKMGVSPGNPVDSHAYPHIQNSKQPRVPSAKAVTSGLPGDTALLLPPSPRPSPRVHLPTQPAADTYSEFHKHYARISTSPSVALSKPYMTVSSEFPAARLSNGKYPKAPEGGEGAQPVPGHARKTAVQDRKDGSSPPLLEKQTVTKDVTDKPLDLSSKVVDVDASKADHMKKMAPTVLVHSRAGSGLVLSGSEIPKETLSPPGNGCAIYRSEIISTAPSSWVVPGPSPNEENNGKSMSLKNKALDWAIPQQRSSSCPRMGGTDAVITNVSGSVSSAGRPASASPAPNANADGTKTSRSSVETTPSVIQHVGQPPATPAKHSSSTSSKGAKASNPEPSFKANENGLPPSSIFLSPNEAFRSPPIPYPRSYLPYPAPEGIAVSPLSLHGKGPVYPHPVLLPNGSLFPGHLAPKPGLPYGLPTGRPEFVTYQDALGLGMVHPMLIPHTPIEITKEEKPERRSRSHERARYEDPTLRNRFSEILETSSTKLHPDVPTDKNLKPNPNWNQGKTVVKSDKLVYVDLLREEPDAKTDTNVSKPSFAAESVGQSAEPPKPSVEPALQQHRDFIALREELGRISDFHETYTFKQPVFTVSKDSVLAGTNKENLGLPVSTPFLEPPLGSDGPAVTFGKTQEDPKPFCVGSAPPSVDVTPTYTKDGADEAESNDGKVLKPKPSKLAKRIANSAGYVGDRFKCVTTELYADSSQLSREQRALQMEGLQEDSILCLPAAYCERAMMRFSELEMKEREGGHPATKDSEMCKFSPADWERLKGNQDKKPKSVTLEEAIAEQNESERCEYSVGNKHRDPFEAPEDKDLPVEKYFVERQPVSEPPADQVASDMPHSPTLRVDRKRKVSGDSSHTETTAEEVPEDPLLKAKRRRVSKDDWPEREMTNSSSNHLEDPHYSELTNLKVCIELTGLHPKKQRHLLHLRERWEQQVSAADGKPGRQSRKEVTQATQPEAIPQGTNITEEKPGRKRAEAKGNRSWSEESLKPSDNEQGLPVFSGSPPMKSLSSTSAGGKKQAQPSCAPASRPPAKQQKIKENQKTDVLCADEEEDCQAASLLQKYTDNSEKPSGKRLCKTKHLIPQESRRGLPLTGEYYVENADGKVTVRRFRKRPEPSSDYDLSPAKQEPKPFDRLQQLLPASQSTQLPCSSSPQETTQSRPMPPEARRLIVNKNAGETLLQRAARLGYEEVVLYCLENKICDVNHRDNAGYCALHEACARGWLNIVRHLLEYGADVNCSAQDGTRPLHDAVENDHLEIVRLLLSYGADPTLATYSGRTIMKMTHSELMEKFLTDYLNDLQGRNDDDASGTWDFYGSSVCEPDDESGYDVLANPPGPEDQDDDDDAYSDVFEFEFSETPLLPCYNIQVSVAQGPRNWLLLSDVLKKLKMSSRIFRCNFPNVEIVTIAEAEFYRQVSASLLFSCSKDLEAFNPESKELLDLVEFTNEIQTLLGSSVEWLHPSDLASDNYW

SEQ ID NO: 21 - DNA, 4096 bp, Homo sapiens
attatcctccttattgacaaacagagcggtcgcggcggcgactctcggcgtgcggtgatagccaagccatgggagacaagaagagccccaccaggccgaagcggcagccgaagccgtcctcggatgagggttactgggactgtagcgtctgcaccttccggaacagcgccgaggccttcaagtgcatgatgtgcgatgtgcggaagggcacctccacccggaaacctcgacctgtctcccagttggttgcacagcaggttactcagcagtttgtgcctcctacacagtcaaagaaagagaaaaaagataaagtagaaaaagaaaaaagtgaaaaggaaacaactagcaaaaagaatagccataagaaaaccaggccaagattgaaaaatgtggatcggagtagtgctcagcatttggaagttactgttggagatctgacagtcattattacagactttaaggagaaaacaaagtcaccgcctgcatctagtgctgcttctgcagatcaacacagtcaaagcggctctagctctgataacacagagagaggaatgtccaggtcatcttcacccagaggagaagcctcatcattgaatggagaatctcattaaagtttattttctccaatttcttagtcacttctgtcctaccatgcaaatacacagattatgccaagaggtaccacattttcatgacagatacattcatgcacaatccataatttgagttttacataaaatagaaatttgttagaatttgttagattttattgcaatgatgcctaccaaacatttccagacttaacattttggtctctgcagttaagtgccatgaaaatgtggttgaattattcattatgcagtgttattggtaagtgtattttcacttttagtttagtgaattctaacacataattcttgaattctctactattggcatgtaacgaatttaaattttttataacatagtgcaagctgcctaaatatgtattatttgagaattgtgaaacagatagttatatgtatacaagtcaaagaacaacttaactattgctgcaacaggtttttcttaatggttatcctcttaaatacacctgctggtacttggtgtggttaaataggaaaattgttattaaataaagaatttgtatgaacctttgccaatgtttttgacacgttttactaattattgttcctgaattatgtttctggttttatctattttttgaggtttttttgtttgcttatttttcaaaacattcatttattgtaatgtttactagcggactagtaaacaataaaacattgattatttagctttataattcaggtttagtgctattgtcattgaacactggtattttctgtatcatataaaacattaaaattcaaataattataagcatttggcaaaaacaagagaaaagaaacttgccatattttacaagctgcaattttagaaaagctttaacttaatgatagttttatcattgttttcttgtcccaaacttatccagggccatagaagtatgaatctaattaaaacagaaatgggaattattgcacagaaatgggaaataactaattttaaatcagtcaaattggcttcttattaaatacaataattcttatgaaaatcatagtaccctattttcagacacagctgccagtttacacatttctcagtatcctgaaaggaaaaaagtatagccccacttatactatgtaaaattaccaataaaatatttttatgactacagattttgcatttttgtttacaactatttaaagagttttatgttgtatttagaatttcaacctagaaaccacacagtacttaaattctcctggggtctcctgctttctcttaaccatttgcttaatatatatctacctaaaggagacttctgaattgtaaatgaacttaaaaatagaatgtggatgcaaaatatcacataagacatcatgataacatttgaagaaaaaataaaactgtagaccctaacagttgtgatatttggtggtttcatgtggtaatgtaattttctgtttaattacagtactttttacaggcacagtggtactgtcttttttgtaagatgctagttgtgaaatacaattaattgcatacagtaaaagtctgtgattaaaacatttatatacctcattctttagtgttgttaaatgaaaaattaaaatttgtgtttattataagatactttcaatggaataccagctaaccagatatgttcctttaaaacatgaatttttttgtcttattctgtttttaacatgtttcaaatgttttatacatttttggagatagtaaaaatttaaaaatgtaatagggaaaatatttaatttttaagtcaaaaactatgctttaaaggaattacatcttatgatcaaataactttcaggatgtattttaaatgagctgcaagagagaaaaatctgacaggagatgggattttacctgaatggagcatactcatatttctacataggtgggaagatactcatctttctacatagacgggaacacggtgtagcatggagattaagcgtgctaactgacacctcatgtttgaatcctgttgtagaatggaaatgagcttaaattacccaagcttactttccatctataaaacagggctaataatggtaatcacatctaatttatagggtttttgggaggattaagtcaatccgtgtaaaattcttaattatctgacacatactggtcactcagtaaatgtgagcttttaccattgttataggtaaaatcccattacaaaaatacaaaaatattttttgtaacagttattttcctgccctcttggatgacctaaagtaatagtgttttatagatttcactaaattttcagtgtaatatcagatgttttctctggatgcagaaagaccatctttccataattaaagaacctggtgggtgtgcactatgagattggagaaatgaattaagttgacttgaaattattttacttttattaatcacaggtatctcaacctgcctttgtttcaccctacttcaagtacacttccacaccaggaaaacagacccaaattccataaacagaactgacgttaaaatatgcgaaagattcagaacctaggtttagggtacatgtttttctctgtttcatgaacttacagtgtggtttgagcactgggttcttttagccaaattccatacaaaaagaatttggatgttttccagcatttattaccttactttgctataagtaagagtgaagataggccgggtgtgatggctcatgccagtaatcccagcactttgggctgaagcaggcagatattttgagcccaggaccagcctgggcaacatggtgaaagctcatctctatgaaaaaatttgaatatacatatatatatatatgtatatttaaaaagtgaatataatttgaggcacagaagaatattcttaaaaccttttgtattatacaatagaaataaaggttttatttttattaaatgctttcctaaaatgatagtggataatagacaaagtgaaaacatttaaaaaggaagagaaacttcagcctttctaagttgatagcatgtttattaacttttagataaagatccttatagactgaaagaatgtagcctctgcattaatggtaaatggtctagaagttttcgtttccactgaggcttccgccactcgcttttttaaacttcctgctatggtttggatatggtttgtcctcaccaaaactcatgctgaggcctgagtccccagtttgattgtgttggtaggtggtgcctttaagaggtgattaggtcattgagatggattaaaggctttctcatgaggctcagttagttctggaatggattcgctcttgcaggaatggatgaattctcacaagagtgggttgttatgaagtgaggatgcttcttgtgttctgttctctttgccctcctcagttcaccatctgctttccaccatgagttgaagcagcatgaggccctcatcagatgggctccctgatcttggactcctcagcctttggactcataagccaaaataaatctgttttctttataaa

SEQ ID NO: 22 - protein, 180 aa, Homo sapiens
MGDKKSPTRPKRQPKPSSDEGYWDCSVCTFRNSAEAFKCMMCDVRKGTSTRKPRPVSQLVAQQVTQQFVPPTQSKKEKKDKVEKEKSEKETTSKKNSHKKTRPRLKNVDRSSAQHLEVTVGDLTVIITDFKEKTKSPPASSAASADQHSQSGSSSDNTERGMSRSSSPRGEASSLNGESH

SEQ ID NO: 23 - DNA, 25 bp, Artificial Sequence
  /note=“Description of Artificial Sequence Synthetic primer”
caccgtcccgcatctgacagcgccg

SEQ ID NO: 24 - DNA, 25 bp, Artificial Sequence
  /note=“Description of Artificial Sequence Synthetic primer”
aaaccggcgctgtcagatgcgggac

SEQ ID NO: 25 - DNA, 25 bp, Artificial Sequence
  /note=“Description of Artificial Sequence Synthetic primer”
caccgggaggcgggatatataccag

SEQ ID NO: 26 - DNA, 25 bp, Artificial Sequence
  /note=“Description of Artificial Sequence Synthetic primer”
aaacctggtatatatcccgcctccc

SEQ ID NO: 27 - DNA, 25 bp, Artificial Sequence
  /note=“Description of Artificial Sequence Synthetic primer”
caccgttgatgacgacgtggtgtca

SEQ ID NO: 28 - DNA, 25 bp, Artificial Sequence
  /note=“Description of Artificial Sequence Synthetic primer”
aaactgacaccacgtcgtcatcaac

SEQ ID NO: 29 - DNA, 25 bp, Artificial Sequence
  /note=“Description of Artificial Sequence Synthetic primer”
caccgggcagtagaacagctcgatg

SEQ ID NO: 30 - DNA, 25 bp, Artificial Sequence
  /note=“Description of Artificial Sequence Synthetic primer”
aaaccatcgagctgttctactgccc

SEQ ID NO: 31 - DNA, 25 bp, Artificial Sequence
  /note=“Description of Artificial Sequence Synthetic primer”
caccgccacgaagtagccggcgcat

SEQ ID NO: 32 - DNA, 25 bp, Artificial Sequence
  /note=“Description of Artificial Sequence Synthetic primer”
aaacatgcgccggctacttcgtggc

SEQ ID NO: 33 - DNA, 25 bp, Artificial Sequence
  /note=“Description of Artificial Sequence Synthetic primer”
caccggctcatcatagcgatagtag

SEQ ID NO: 34 - DNA, 25 bp, Artificial Sequence
  /note=“Description of Artificial Sequence Synthetic primer”
aaacctactatcgctatgatgagcc

SEQ ID NO: 35 - DNA, 25 bp, Artificial Sequence
  /note=“Description of Artificial Sequence Synthetic primer”
caccgtgcggcgtaatgcttgaaag

SEQ ID NO: 36 - DNA, 25 bp, Artificial Sequence
  /note=“Description of Artificial Sequence Synthetic primer”
aaacctttcaagcattacgccgcac

SEQ ID NO: 37 - DNA, 25 bp, Artificial Sequence
  /note=“Description of Artificial Sequence Synthetic primer”
caccgggattaattcgctaaatgat

SEQ ID NO: 38 - DNA, 25 bp, Artificial Sequence
  /note=“Description of Artificial Sequence Synthetic primer”
aaacatcatttagcgaattaatccc

SEQ ID NO: 39 - DNA, 20 bp, Artificial Sequence
  /note=“Description of Artificial Sequence Synthetic primer”
tctctgctggatgacgtgag

SEQ ID NO: 40 - DNA, 19 bp, Artificial Sequence
  /note=“Description of Artificial Sequence Synthetic primer”
tagctgtgctcgcgctact

SEQ ID NO: 41 - DNA, 20 bp, Artificial Sequence
  /note=“Description of Artificial Sequence Synthetic primer”
tcagggctttcgtacaggag

SEQ ID NO: 42 - DNA, 20 bp, Artificial Sequence
  /note=“Description of Artificial Sequence Synthetic primer”
tccggaaaccgtgtgtactt

SEQ ID NO: 43 - DNA, 19 bp, Artificial Sequence
  /note=“Description of Artificial Sequence Synthetic primer”
actgcatcctggatctccc

SEQ ID NO: 44 - DNA, 20 bp, Artificial Sequence
  /note=“Description of Artificial Sequence Synthetic primer”
tcgactcaccctcctttctc

SEQ ID NO: 45 - DNA, 22 bp, Artificial Sequence
  /note=“Description of Artificial Sequence Synthetic primer”
accctggaggtagcaggtcttt

SEQ ID NO: 46 - DNA, 23 bp, Artificial Sequence
  /note=“Description of Artificial Sequence Synthetic primer”
aatccttgcaggtggacaggtag

SEQ ID NO: 47 - DNA, 20 bp, Artificial Sequence
  /note=“Description of Artificial Sequence Synthetic primer”
tcaaacactcggttcaccac

SEQ ID NO: 48 - DNA, 18 bp, Artificial Sequence
  /note=“Description of Artificial Sequence Synthetic primer”
ggagaagtccacaccggg

SEQ ID NO: 49 - DNA, 19 bp, Artificial Sequence
  /note=“Description of Artificial Sequence Synthetic primer”
catgggccatctcaatctg

SEQ ID NO: 50 - DNA, 20 bp, Artificial Sequence
  /note=“Description of Artificial Sequence Synthetic primer”
tctccagagctcgctttacc

SEQ ID NO: 51 - DNA, 20 bp, Artificial Sequence
  /note=“Description of Artificial Sequence Synthetic primer”
gcggctactacaaccagagc

SEQ ID NO: 52 - DNA, 20 bp, Artificial Sequence
  /note=“Description of Artificial Sequence Synthetic primer”
gatgtaatccttgccgtcgt

SEQ ID NO: 53 - DNA, 26 bp, Artificial Sequence
  /note=“Description of Artificial Sequence Synthetic primer”
gacggcaaggattacatcgccctgaa

SEQ ID NO: 54 - DNA, 18 bp, Artificial Sequence
  /note=“Description of Artificial Sequence Synthetic primer”
cacgggccgcctcccact

SEQ ID NO: 55 - DNA, 21 bp, Artificial Sequence
  /note=“Description of Artificial Sequence Synthetic primer”
ggagacacagaagtacaagcg

SEQ ID NO: 56 - DNA, 22 bp, Artificial Sequence
  /note=“Description of Artificial Sequence Synthetic primer”
cgtcgtaggcgtactggtcata

SEQ ID NO: 57 - DNA, 16 bp, Artificial Sequence
  /note=“Description of Artificial Sequence Synthetic primer”
cctacgacggcaagga

SEQ ID NO: 58 - DNA, 20 bp, Artificial Sequence
  /note=“Description of Artificial Sequence Synthetic primer”
cccttctccaggtatttgtg

SEQ ID NO: 59 - DNA, 20 bp, Artificial Sequence
  /note=“Description of Artificial Sequence Synthetic primer”
cgctctgtggccactttcag

SEQ ID NO: 60 - DNA, 20 bp, Artificial Sequence
  /note=“Description of Artificial Sequence Synthetic primer”
tgcccgctgtgagacttcat

SEQ ID NO: 61 - DNA, 19 bp, Artificial Sequence
  /note=“Description of Artificial Sequence Synthetic primer”
atttgggtcgcggttcttg

SEQ ID NO: 62 - DNA, 21 bp, Artificial Sequence
  /note=“Description of Artificial Sequence Synthetic primer”
tgccttgacattctcgatggt

SEQ ID NO: 63 - DNA, 20 bp, Artificial Sequence
  /note=“Description of Artificial Sequence Synthetic primer”
tgcaccaccaactgcttagc

SEQ ID NO: 64 - DNA, 21 bp, Artificial Sequence
  /note=“Description of Artificial Sequence Synthetic primer”
ggcatggactgtggtcatgag

SEQ ID NO: 65 - DNA, 21 bp, Artificial Sequence
  /note=“Description of Artificial Sequence Synthetic primer”
tgacactggcaaaacaatgca

SEQ ID NO: 66 - DNA, 21 bp, Artificial Sequence
  /note=“Description of Artificial Sequence Synthetic primer”
ggtccttttcaccagcaagct

SEQ ID NO: 67 - DNA, 20 bp, Artificial Sequence
  /note=“Description of Artificial Sequence Synthetic primer”
ctggaacggtgaaggtgaca

SEQ ID NO: 68 - DNA, 23 bp, Artificial Sequence
  /note=“Description of Artificial Sequence Synthetic primer”
aagggacttcctgtaacaatgca

SEQ ID NO: 69 - protein, 9 aa, Artificial Sequence
  /note=“Description of Artificial Sequence Synthetic peptide sequence”
TSDKAIELY

SEQ ID NO: 70 - DNA, 26 bp, Artificial Sequence
  /note=“Description of Artificial Sequence Synthetic primer”
gatacaaggctgttagagagataatt

SEQ ID NO: 71 - DNA, 21 bp, Artificial Sequence
  /note=“Description of Artificial Sequence Synthetic primer”
gaccaagaggaagaatcacaa

SEQ ID NO: 72 - DNA, 21 bp, Artificial Sequence
  /note=“Description of Artificial Sequence Synthetic primer”
gctgcgaagaagctcgttaga

SEQ ID NO: 73 - DNA, 21 bp, Artificial Sequence
  /note=“Description of Artificial Sequence Synthetic primer”
ggagcagcttacaccaattga

SEQ ID NO: 74 - DNA, 57 bp, Artificial Sequence
  /note=“Description of Artificial Sequence Synthetic primer”
ccggaccaagaggaagaatcacaatcaagagttgtgattcttcctcttggtcttttt

SEQ ID NO: 75 - DNA, 56 bp, Artificial Sequence
  /note=“Description of Artificial Sequence Synthetic primer”
aattaaaaagaccaagaggaagaatcacaactcttgattgtgattcttcctcttgg

SEQ ID NO: 76 - DNA, 57 bp, Artificial Sequence
  /note=“Description of Artificial Sequence Synthetic primer”
ccggctgcgaagaagctcgttagatcaagagtctaacgagcttcttcgcagcttttt

SEQ ID NO: 77 - DNA, 57 bp, Artificial Sequence
  /note=“Description of Artificial Sequence Synthetic primer”
aattaaaaagctgcgaagaagctcgttagactcttgatctaacgagcttcttcgcag

SEQ ID NO: 78 - DNA, 56 bp, Artificial Sequence
  /note=“Description of Artificial Sequence Synthetic primer”
ccggagcagcttacaccaattgatcaagagtcaattggtgtaagctgctccttttt

SEQ ID NO: 79 - DNA, 56 bp, Artificial Sequence
  /note=“Description of Artificial Sequence Synthetic primer”
aattaaaaaggagcagcttacaccaattgactcttgatcaattggtgtaagctgct

SEQ ID NO: 80 - DNA, 26 bp, Artificial Sequence
  /note=“Description of Artificial Sequence Synthetic primer”
gatacaaggctgttagagagataatt

SEQ ID NO: 81 - DNA, 21 bp, Artificial Sequence
  /note=“Description of Artificial Sequence Synthetic primer”
ccaacgaccaactccctaaat

SEQ ID NO: 82 - DNA, 24 bp, Artificial Sequence
  /note=“Description of Artificial Sequence Synthetic primer”
aaggcactgtagtttgaggtatag

SEQ ID NO: 83 - DNA, 20 bp, Artificial Sequence
  /note=“Description of Artificial Sequence Synthetic primer”
tcgcctccttcatcacacta

SEQ ID NO: 84 - DNA, 18 bp, Artificial Sequence
  /note=“Description of Artificial Sequence Synthetic primer”
cgagtccacgtgagggaa

SEQ ID NO: 85 - DNA, 20 bp, Artificial Sequence
  /note=“Description of Artificial Sequence Synthetic primer”
cttcttccttccggctttct

SEQ ID NO: 86 - DNA, 20 bp, Artificial Sequence
  /note=“Description of Artificial Sequence Synthetic primer”
agctgggagaggacacacac

SEQ ID NO: 87 - DNA, 11 bp, Artificial Sequence
  /note=“Description of Artificial Sequence Synthetic primer”
cgcagcacaag

SEQ ID NO: 88 - DNA, 22 bp, Artificial Sequence
  /note=“Description of Artificial Sequence Synthetic primer”
gccgatgcaggagtcgcacagc